SEQUENCE LISTING (231 sequences)

SEQ ID NO: 1 - protein, 461 aa, Homo sapiens
  REGION 1..28: note = MISC_FEATURE - Signal peptide
  REGION 29..46: note = MISC_FEATURE - Propeptide
  REGION 47..461: note = MISC_FEATURE - Mature FIX
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 2 - DNA, 2800 bp, Homo sapiens
  source 1..2800: mol_type = other DNA
actttcacaatctgctagcaaaggttatgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaatgaaagatggatttccaaggttaattcattggaattgaaaattaacagggcctctcactaactaatcactttcccatcttttgttagatttgaatatatacattctatgatcattgctttttctctttacaggggagaatttcatattttacctgagcaaattgattagaaaatggaaccactagaggaatataatgtgttaggaaattacagtcatttctaagggcccagcccttgacaaaattgtgaagttaaattctccactctgtccatcagatactatggttctccactatggcaactaactcactcaattttccctccttagcagcattccatcttcccgatcttctttgcttctccaaccaaaacatcaatgtttattagttctgtatacagtacaggatctttggtctactctatcacaaggccagtaccacactcatgaagaaagaacacaggagtagctgagaggctaaaactcatcaaaaacactactccttttcctctaccctattcctcaatcttttaccttttccaaatcccaatccccaaatcagtttttctctttcttactccctctctcccttttaccctccatggtcgttaaaggagagatggggagcatcattctgttatacttctgtacacagttatacatgtctatcaaacccagacttgcttccgtagtggagacttgcttttcagaacatagggatgaagtaaggtgcctgaaaagtttgggggaaaagtttctttcagagagttaagttattttatatatataatatatatataaaatatataatatacaatataaatatatagtgtgtgtgtatgcgtgtgtgtagacacacacgcatacacacatataatggaagcaataagccattctaagagcttgtatggttatggaggtctgactaggcatgatttcacgaaggcaagattggcatatcattgtaactaaaaaagctgacattgacccagacatattgtactctttctaaaaataataataataatgctaacagaaagaagagaaccgttcgtttgcaatctacagctagtagagactttgaggaagaattcaacagtgtgtcttcagcagtgttcagagccaagcaagaagttgaagttgcctagaccagaggacataagtatcatgtctcctttaactagcataccccgaagtggagaagggtgcagcaggctcaaaggcataagtcattccaatcagccaactaagttgtccttttctggtttcgtgttcaccatggaacattttgattatagttaatccttctatcttgaatcttctagagagttgctgaccaactgacgtatgtttccctttgtgaattaataaactggtgttctggttcata

SEQ ID NO: 3 - DNA, 1386 bp, Homo sapiens
  source 1..1386: mol_type = other DNA
atgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaa

SEQ ID NO: 4 - DNA, 709 bp, Homo sapiens
  source 1..709: mol_type = other DNA
gtaagaaatccatttttctattgttcaacttttattctattttcccagtaaaataaagttttagtaaactctgcatctttaaagaattattttggcatttatttctaaaatggcatagtattttgtatttgtgaagtcttacaaggttatcttattaataaaattcaaacatcctaggtaaaaaaaaaaaaaggtcagaattgtttagtgactgtaattttcttttgcgcactaaggaaagtgcaaagtaacttagagtgactgaaacttcacagaatagggttgaagattgaattcataactatcccaaagacctatccattgcactatgctttatttaaaaaccacaaaacctgtgctgttgatctcataaatagaacttgtatttatatttattttcattttagtctgtcttcttggttgctgttgatagacactaaaagagtattagatattatctaagtttgaatataaggctataaatatttaataatttttaaaatagtattcttggtaattgaattattcttctgtttaaaggcagaagaaataattgaacatcatcctgagtttttctgtaggaatcagagcccaatattttgaaacaaatgcataatctaagtcaaatggaaagaaatataaaaagtaacattattacttcttgttttcttcagtatttaacaatccttttttttcttcccttgcccag

SEQ ID NO: 5-7 - skipped (3 entries)

SEQ ID NO: 8 - protein, 1391 aa, synthetic construct
  REGION 1..1391: note = Synthetic
MDKPKKKRKVKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKK

SEQ ID NO: 9 - DNA, 4176 bp, synthetic construct
  misc_feature 1..4176: note = Synthetic
  source 1..4176: mol_type = other DNA
atggacaagcccaagaaaaagcggaaagtgaagtacagcatcggcctggacatcggcaccaactctgtgggctgggccgtgatcaccgacgagtacaaggtgcccagcaagaaattcaaggtgctgggcaacaccgacaggcacagcatcaagaagaacctgatcggcgccctgctgttcgacagcggcgaaacagccgaggccaccagactgaagagaaccgccagaagaagatacaccaggcggaagaacaggatctgctatctgcaagagatcttcagcaacgagatggccaaggtggacgacagcttcttccacagactggaagagtccttcctggtggaagaggacaagaagcacgagagacaccccatcttcggcaacatcgtggacgaggtggcctaccacgagaagtaccccaccatctaccacctgagaaagaaactggtggacagcaccgacaaggccgacctgagactgatctacctggccctggcccacatgatcaagttcagaggccacttcctgatcgagggcgacctgaaccccgacaacagcgacgtggacaagctgttcatccagctggtgcagacctacaaccagctgttcgaggaaaaccccatcaacgccagcggcgtggacgccaaggctatcctgtctgccagactgagcaagagcagaaggctggaaaatctgatcgcccagctgcccggcgagaagaagaacggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgttcctggccgccaagaacctgtctgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagaaatcttcttcgaccagagcaagaacggctacgccggctacatcgatggcggcgctagccaggaagagttctacaagttcatcaagcccatcctggaaaagatggacggcaccgaggaactgctcgtgaagctgaacagagaggacctgctgagaaagcagagaaccttcgacaacggcagcatcccccaccagatccacctgggagagctgcacgctatcctgagaaggcaggaagatttttacccattcctgaaggacaaccgggaaaagatcgagaagatcctgaccttcaggatcccctactacgtgggccccctggccagaggcaacagcagattcgcctggatgaccagaaagagcgaggaaaccatcaccccctggaacttcgaggaagtggtggacaagggcgccagcgcccagagcttcatcgagagaatgacaaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaggccatcgtggacctgctgttcaagaccaacagaaaagtgaccgtgaagcagctgaaagaggactacttcaagaaaatcgagtgcttcgactccgtggaaatctccggcgtggaagatagattcaacgcctccctgggcacataccacgatctgctgaaaattatcaaggacaaggacttcctggataacgaagagaacgaggacattctggaagatatcgtgctgaccctgacactgtttgaggaccgcgagatgatcgaggaaaggctgaaaacctacgctcacctgttcgacgacaaagtgatgaagcagctgaagagaaggcggtacaccggctggggcaggctgagcagaaagctgatcaacggcatcagagacaagcagagcggcaagacaatcctggatttcctgaagtccgacggcttcgccaaccggaacttcatgcagctgatccacgacgacagcctgacattcaaagaggacatccagaaagcccaggtgtccggccagggcgactctctgcacgagcatatcgctaacctggccggcagccccgctatcaagaagggcatcctgcagacagtgaaggtggtggacgagctcgtgaaagtgatgggcagacacaagcccgagaacatcgtgatcgagatggctagagagaaccagaccacccagaagggacagaagaactcccgcgagaggatgaagagaatcgaagagggcatcaaagagctgggcagccagatcctgaaagaacaccccgtggaaaacacccagctgcagaacgagaagctgtacctgtactacctgcagaatggccgggatatgtacgtggaccaggaactggacatcaacagactgtccgactacgatgtggaccatatcgtgcctcagagctttctgaaggacgactccatcgataacaaagtgctgactcggagcgacaagaacagaggcaagagcgacaacgtgccctccgaagaggtcgtgaagaagatgaagaactactggcgacagctgctgaacgccaagctgattacccagaggaagttcgataacctgaccaaggccgagagaggcggcctgagcgagctggataaggccggcttcatcaagaggcagctggtggaaaccagacagatcacaaagcacgtggcacagatcctggactcccggatgaacactaagtacgacgaaaacgataagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgaaatcaccctggccaacggcgagatcagaaagcgccctctgatcgagacaaacggcgaaaccggggagatcgtgtgggataagggcagagacttcgccacagtgcgaaaggtgctgagcatgccccaagtgaatatcgtgaaaaagaccgaggtgcagacaggcggcttcagcaaagagtctatcctgcccaagaggaacagcgacaagctgatcgccagaaagaaggactgggaccccaagaagtacggcggcttcgacagccctaccgtggcctactctgtgctggtggtggctaaggtggaaaagggcaagtccaagaaactgaagagtgtgaaagagctgctggggatcaccatcatggaaagaagcagctttgagaagaaccctatcgactttctggaagccaagggctacaaagaagtgaaaaaggacctgatcatcaagctgcctaagtactccctgttcgagctggaaaacggcagaaagagaatgctggcctctgccggcgaactgcagaagggaaacgagctggccctgcctagcaaatatgtgaacttcctgtacctggcctcccactatgagaagctgaagggcagccctgaggacaacgaacagaaacagctgtttgtggaacagcataagcactacctggacgagatcatcgagcagatcagcgagttctccaagagagtgatcctggccgacgccaatctggacaaggtgctgtctgcctacaacaagcacagggacaagcctatcagagagcaggccgagaatatcatccacctgttcaccctgacaaacctgggcgctcctgccgccttcaagtactttgacaccaccatcgaccggaagaggtacaccagcaccaaagaggtgctggacgccaccctgatccaccagagcatcaccggcctgtacgagacaagaatcgacctgtctcagctgggaggcgacaagagacctgccgccactaagaaggccggacaggccaaaaagaagaagtga

SEQ ID NO: 10 - DNA, 4218 bp, synthetic construct
  misc_feature 1..4218: note = Synthetic
  source 1..4218: mol_type = other DNA
atggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggacaagaagcacgagagacaccccatcttcggcaacatcgtggacgaggtggcctaccacgagaagtaccccaccatctaccacctgagaaagaaactggtggacagcaccgacaaggccgacctgagactgatctacctggccctggcccacatgatcaagttcagaggccacttcctgatcgagggcgacctgaaccccgacaacagcgacgtggacaagctgttcatccagctggtgcagacctacaaccagctgttcgaggaaaaccccatcaacgccagcggcgtggacgccaaggctatcctgtctgccagactgagcaagagcagaaggctggaaaatctgatcgcccagctgcccggcgagaagaagaacggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgttcctggccgccaagaacctgtctgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagaaatcttcttcgaccagagcaagaacggctacgccggctacatcgatggcggcgctagccaggaagagttctacaagttcatcaagcccatcctggaaaagatggacggcaccgaggaactgctcgtgaagctgaacagagaggacctgctgagaaagcagagaaccttcgacaacggcagcatcccccaccagatccacctgggagagctgcacgctatcctgagaaggcaggaagatttttacccattcctgaaggacaaccgggaaaagatcgagaagatcctgaccttcaggatcccctactacgtgggccccctggccagaggcaacagcagattcgcctggatgaccagaaagagcgaggaaaccatcaccccctggaacttcgaggaagtggtggacaagggcgccagcgcccagagcttcatcgagagaatgacaaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaggccatcgtggacctgctgttcaagaccaacagaaaagtgaccgtgaagcagctgaaagaggactacttcaagaaaatcgagtgcttcgactccgtggaaatctccggcgtggaagatagattcaacgcctccctgggcacataccacgatctgctgaaaattatcaaggacaaggacttcctggataacgaagagaacgaggacattctggaagatatcgtgctgaccctgacactgtttgaggaccgcgagatgatcgaggaaaggctgaaaacctacgctcacctgttcgacgacaaagtgatgaagcagctgaagagaaggcggtacaccggctggggcaggctgagcagaaagctgatcaacggcatcagagacaagcagagcggcaagacaatcctggatttcctgaagtccgacggcttcgccaaccggaacttcatgcagctgatccacgacgacagcctgacattcaaagaggacatccagaaagcccaggtgtccggccagggcgactctctgcacgagcatatcgctaacctggccggcagccccgctatcaagaagggcatcctgcagacagtgaaggtggtggacgagctcgtgaaagtgatgggcagacacaagcccgagaacatcgtgatcgagatggctagagagaaccagaccacccagaagggacagaagaactcccgcgagaggatgaagagaatcgaagagggcatcaaagagctgggcagccagatcctgaaagaacaccccgtggaaaacacccagctgcagaacgagaagctgtacctgtactacctgcagaatggccgggatatgtacgtggaccaggaactggacatcaacagactgtccgactacgatgtggaccatatcgtgcctcagagctttctgaaggacgactccatcgataacaaagtgctgactcggagcgacaagaacagaggcaagagcgacaacgtgccctccgaagaggtcgtgaagaagatgaagaactactggcgacagctgctgaacgccaagctgattacccagaggaagttcgataacctgaccaaggccgagagaggcggcctgagcgagctggataaggccggcttcatcaagaggcagctggtggaaaccagacagatcacaaagcacgtggcacagatcctggactcccggatgaacactaagtacgacgaaaacgataagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgaaatcaccctggccaacggcgagatcagaaagcgccctctgatcgagacaaacggcgaaaccggggagatcgtgtgggataagggcagagacttcgccacagtgcgaaaggtgctgagcatgccccaagtgaatatcgtgaaaaagaccgaggtgcagacaggcggcttcagcaaagagtctatcctgcccaagaggaacagcgacaagctgatcgccagaaagaaggactgggaccccaagaagtacggcggcttcgacagccctaccgtggcctactctgtgctggtggtggctaaggtggaaaagggcaagtccaagaaactgaagagtgtgaaagagctgctggggatcaccatcatggaaagaagcagctttgagaagaaccctatcgactttctggaagccaagggctacaaagaagtgaaaaaggacctgatcatcaagctgcctaagtactccctgttcgagctggaaaacggcagaaagagaatgctggcctctgccggcgaactgcagaagggaaacgagctggccctgcctagcaaatatgtgaacttcctgtacctggcctcccactatgagaagctgaagggcagccctgaggacaacgaacagaaacagctgtttgtggaacagcataagcactacctggacgagatcatcgagcagatcagcgagttctccaagagagtgatcctggccgacgccaatctggacaaggtgctgtctgcctacaacaagcacagggacaagcctatcagagagcaggccgagaatatcatccacctgttcaccctgacaaacctgggcgctcctgccgccttcaagtactttgacaccaccatcgaccggaagaggtacaccagcaccaaagaggtgctggacgccaccctgatccaccagagcatcaccggcctgtacgagacaagaatcgacctgtctcagctgggaggcgacaagagacctgccgccactaagaaggccggacaggccaaaaagaagaagtgagcggccgcttaatta

SEQ ID NO: 11 - protein, 1379 aa, synthetic construct
  REGION 1..1379: note = Synthetic
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDGGGSPKKKRKV

SEQ ID NO: 12 - RNA, 4514 nt, synthetic construct
  misc_feature 1..4514: note = Synthetic
  source 1..4514: mol_type = other RNA
gggtcccgcagtcggcgtccagcggctctgcttgttcgtgtgtgtgtcgttgcaggccttattcggatccatggataagaagtactcaatcgggctggatatcggaactaattccgtgggttgggcagtgatcacggatgaatacaaagtgccgtccaagaagttcaaggtcctggggaacaccgatagacacagcatcaagaaaaatctcatcggagccctgctgtttgactccggcgaaaccgcagaagcgacccggctcaaacgtaccgcgaggcgacgctacacccggcggaagaatcgcatctgctatctgcaagagatcttttcgaacgaaatggcaaaggtcgacgacagcttcttccaccgcctggaagaatctttcctggtggaggaggacaagaagcatgaacggcatcctatctttggaaacatcgtcgacgaagtggcgtaccacgaaaagtacccgaccatctaccatctgcggaagaagttggttgactcaactgacaaggccgacctcagattgatctacttggccctcgcccatatgatcaaattccgcggacacttcctgatcgaaggcgatctgaaccctgataactccgacgtggataagcttttcattcaactggtgcagacctacaaccaactgttcgaagaaaacccaatcaatgctagcggcgtcgatgccaaggccatcctgtccgcccggctgtcgaagtcgcggcgcctcgaaaacctgatcgcacagctgccgggagagaaaaagaacggacttttcggcaacttgatcgctctctcactgggactcactcccaatttcaagtccaattttgacctggccgaggacgcgaagctgcaactctcaaaggacacctacgacgacgacttggacaatttgctggcacaaattggcgatcagtacgcggatctgttccttgccgctaagaacctttcggacgcaatcttgctgtccgatatcctgcgcgtgaacaccgaaataaccaaagcgccgcttagcgcctcgatgattaagcggtacgacgagcatcaccaggatctcacgctgctcaaagcgctcgtgagacagcaactgcctgaaaagtacaaggagatcttcttcgaccagtccaagaatgggtacgcagggtacatcgatggaggcgctagccaggaagagttctataagttcatcaagccaatcctggaaaagatggacggaaccgaagaactgctggtcaagctgaacagggaggatctgctccggaaacagagaacctttgacaacggatccattccccaccagatccatctgggtgagctgcacgccatcttgcggcgccaggaggacttttacccattcctcaaggacaaccgggaaaagatcgagaaaattctgacgttccgcatcccgtattacgtgggcccactggcgcgcggcaattcgcgcttcgcgtggatgactagaaaatcagaggaaaccatcactccttggaatttcgaggaagttgtggataagggagcttcggcacaaagcttcatcgaacgaatgaccaacttcgacaagaatctcccaaacgagaaggtgcttcctaagcacagcctcctttacgaatacttcactgtctacaacgaactgactaaagtgaaatacgttactgaaggaatgaggaagccggcctttctgtccggagaacagaagaaagcaattgtcgatctgctgttcaagaccaaccgcaaggtgaccgtcaagcagcttaaagaggactacttcaagaagatcgagtgtttcgactcagtggaaatcagcggggtggaggacagattcaacgcttcgctgggaacctatcatgatctcctgaagatcatcaaggacaaggacttccttgacaacgaggagaacgaggacatcctggaagatatcgtcctgaccttgacccttttcgaggatcgcgagatgatcgaggagaggcttaagacctacgctcatctcttcgacgataaggtcatgaaacaactcaagcgccgccggtacactggttggggccgcctctcccgcaagctgatcaacggtattcgcgataaacagagcggtaaaactatcctggatttcctcaaatcggatggcttcgctaatcgtaacttcatgcaattgatccacgacgacagcctgacctttaaggaggacatccaaaaagcacaagtgtccggacagggagactcactccatgaacacatcgcgaatctggccggttcgccggcgattaagaagggaattctgcaaactgtgaaggtggtcgacgagctggtgaaggtcatgggacggcacaaaccggagaatatcgtgattgaaatggcccgagaaaaccagactacccagaagggccagaaaaactcccgcgaaaggatgaagcggatcgaagaaggaatcaaggagctgggcagccagatcctgaaagagcacccggtggaaaacacgcagctgcagaacgagaagctctacctgtactatttgcaaaatggacgggacatgtacgtggaccaagagctggacatcaatcggttgtctgattacgacgtggaccacatcgttccacagtcctttctgaaggatgactcgatcgataacaaggtgttgactcgcagcgacaagaacagagggaagtcagataatgtgccatcggaggaggtcgtgaagaagatgaagaattactggcggcagctcctgaatgcgaagctgattacccagagaaagtttgacaatctcactaaagccgagcgcggcggactctcagagctggataaggctggattcatcaaacggcagctggtcgagactcggcagattaccaagcacgtggcgcagatcttggactcccgcatgaacactaaatacgacgagaacgataagctcatccgggaagtgaaggtgattaccctgaaaagcaaacttgtgtcggactttcggaaggactttcagttttacaaagtgagagaaatcaacaactaccatcacgcgcatgacgcatacctcaacgctgtggtcggtaccgccctgatcaaaaagtaccctaaacttgaatcggagtttgtgtacggagactacaaggtctacgacgtgaggaagatgatagccaagtccgaacaggaaatcgggaaagcaactgcgaaatacttcttttactcaaacatcatgaactttttcaagactgaaattacgctggccaatggagaaatcaggaagaggccactgatcgaaactaacggagaaacgggcgaaatcgtgtgggacaagggcagggacttcgcaactgttcgcaaagtgctctctatgccgcaagtcaatattgtgaagaaaaccgaagtgcaaaccggcggattttcaaaggaatcgatcctcccaaagagaaatagcgacaagctcattgcacgcaagaaagactgggacccgaagaagtacggaggattcgattcgccgactgtcgcatactccgtcctcgtggtggccaaggtggagaagggaaagagcaaaaagctcaaatccgtcaaagagctgctggggattaccatcatggaacgatcctcgttcgagaagaacccgattgatttcctcgaggcgaagggttacaaggaggtgaagaaggatctgatcatcaaactccccaagtactcactgttcgaactggaaaatggtcggaagcgcatgctggcttcggccggagaactccaaaaaggaaatgagctggccttgcctagcaagtacgtcaacttcctctatcttgcttcgcactacgaaaaactcaaagggtcaccggaagataacgaacagaagcagcttttcgtggagcagcacaagcattatctggatgaaatcatcgaacaaatctccgagttttcaaagcgcgtgatcctcgccgacgccaacctcgacaaagtcctgtcggcctacaataagcatagagataagccgatcagagaacaggccgagaacattatccacttgttcaccctgactaacctgggagccccagccgccttcaagtacttcgatactactatcgatcgcaaaagatacacgtccaccaaggaagttctggacgcgaccctgatccaccaaagcatcactggactctacgaaactaggatcgatctgtcgcagctgggtggcgatggcggtggatctccgaaaaagaagagaaaggtgtaatgagctagccatcacatttaaaagcatctcagcctaccatgagaataagagaaagaaaatgaagatcaatagcttattcatctctttttctttttcgttggtgtaaagccaacaccctgtctaaaaaacataaatttctttaatcattttgcctcttttctctgtgcttcaattaataaaaaatggaaagaacctcgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 13 - protein, 7 aa, synthetic construct
  REGION 1..7: note = Synthetic
PKKKRKV

SEQ ID NO: 14 - protein, 7 aa, synthetic construct
  REGION 1..7: note = Synthetic
PKKKRRV

SEQ ID NO: 15 - protein, 16 aa, synthetic construct
  REGION 1..16: note = Synthetic
KRPAATKKAGQAKKKK

SEQ ID NO: 16 - RNA, 16 nt, synthetic construct
  misc_feature 1..16: note = Synthetic
  source 1..16: mol_type = other RNA
gttttagagctatgct

SEQ ID NO: 17 - RNA, 22 nt, synthetic construct
  misc_feature 1..22: note = Synthetic
  source 1..22: mol_type = other RNA
gttttagagctatgctgttttg

SEQ ID NO: 18 - RNA, 67 nt, synthetic construct
  misc_feature 1..67: note = Synthetic
  source 1..67: mol_type = other RNA
agcatagcaagttaaaataaggctagtccgttatcaacttgaaaaagtggcaccgagtcggtgcttt

SEQ ID NO: 19 - RNA, 72 nt, synthetic construct
  misc_feature 1..72: note = Synthetic
  source 1..72: mol_type = other RNA
aaacagcatagcaagttaaaataaggctagtccgttatcaacttgaaaaagtggcaccgagtcggtgctttt

SEQ ID NO: 20 - RNA, 82 nt, synthetic construct
  misc_feature 1..82: note = Synthetic
  source 1..82: mol_type = other RNA
gttggaaccattcaaaacagcatagcaagttaaaataaggctagtccgttatcaacttgaaaaagtggcaccgagtcggtgc

SEQ ID NO: 21 - RNA, 77 nt, synthetic construct
  misc_feature 1..77: note = Synthetic
  source 1..77: mol_type = other RNA
gttttagagctagaaatagcaagttaaaataaggctagtccgttatcaacttgaaaaagtggcaccgagtcggtgct

SEQ ID NO: 22 - RNA, 82 nt, synthetic construct; identical to SEQ ID NO: 20
  misc_feature 1..82: note = Synthetic
  source 1..82: mol_type = other RNA

SEQ ID NO: 23 - RNA, 76 nt, synthetic construct
  misc_feature 1..76: note = Synthetic
  source 1..76: mol_type = other RNA
gttttagagctagaaatagcaagttaaaataaggctagtccgttatcaacttgaaaaagtggcaccgagtcggtgc

SEQ ID NO: 24 - RNA, 86 nt, synthetic construct
  misc_feature 1..86: note = Synthetic
  source 1..86: mol_type = other RNA
gtttaagagctatgctggaaacagcatagcaagtttaaataaggctagtccgttatcaacttgaaaaagtggcaccgagtcggtgc

SEQ ID NO: 25 - RNA, 83 nt, synthetic construct
  misc_feature 1..83: note = Synthetic
  source 1..83: mol_type = other RNA
gttttagagctagaaatagcaagttaaaataaggctagtccgttatcaacttgaaaaagtggcaccgagtcggtgcttttttt

SEQ ID NO: 26 - RNA, 80 nt, synthetic construct
  misc_feature 1..80: note = Synthetic
  source 1..80: mol_type = other RNA
gttttagagctagaaatagcaagttaaaataaggctagtccgttatcaacttgaaaaagtggcaccgagtcggtgctttt

SEQ ID NO: 27 - RNA, 92 nt, synthetic construct
  misc_feature 1..92: note = Synthetic
  source 1..92: mol_type = other RNA
gtttaagagctatgctggaaacagcatagcaagtttaaataaggctagtccgttatcaacttgaaaaagtggcaccgagtcggtgctttttt

SEQ ID NO: 28 - RNA, 68 nt, synthetic construct
  misc_feature 1..68: note = Synthetic
  source 1..68: mol_type = other RNA
gttttagagctagaaatagcaagttaaaataaggctagtccgttatcaacttggcaccgagtcggtgc

SEQ ID NO: 29 - RNA, 100 nt, synthetic construct
  misc_feature 1..100: note = Synthetic
  misc_difference 1..20: note = n is a, c, g, or u
  source 1..100: mol_type = other RNA
nnnnnnnnnnnnnnnnnnnngttttagagctagaaatagcaagttaaaataaggctagtccgttatcaacttgaaaaagtggcaccgagtcggtgctttt

SEQ ID NO: 30 - RNA, 20 nt, synthetic construct
  misc_feature 1..20: note = Synthetic
  source 1..20: mol_type = other RNA
gagcaacctcactcttgtct

SEQ ID NO: 31 - RNA, 20 nt, synthetic construct
  misc_feature 1..20: note = Synthetic
  source 1..20: mol_type = other RNA
atgcatttgtttcaaaatat

SEQ ID NO: 32 - RNA, 20 nt, synthetic construct
  misc_feature 1..20: note = Synthetic
  source 1..20: mol_type = other RNA
tgcatttgtttcaaaatatt

SEQ ID NO: 33 - RNA, 20 nt, synthetic construct
  misc_feature 1..20: note = Synthetic
  source 1..20: mol_type = other RNA
atttatgagatcaacagcac

SEQ ID NO: 34 - RNA, 20 nt, synthetic construct
  misc_feature 1..20: note = Synthetic
  source 1..20: mol_type = other RNA
gatcaacagcacaggttttg

SEQ ID NO: 35 - RNA, 20 nt, synthetic construct
  misc_feature 1..20: note = Synthetic
  source 1..20: mol_type = other RNA
ttaaataaagcatagtgcaa

SEQ ID NO: 36 - RNA, 20 nt, synthetic construct
  misc_feature 1..20: note = Synthetic
  source 1..20: mol_type = other RNA
taaagcatagtgcaatggat

SEQ ID NO: 37 - RNA, 20 nt, synthetic construct
  misc_feature 1..20: note = Synthetic
  source 1..20: mol_type = other RNA
tagtgcaatggataggtctt

SEQ ID NO: 38 - RNA, 20 nt, synthetic construct
  misc_feature 1..20: note = Synthetic
  source 1..20: mol_type = other RNA
tactaaaactttattttact

SEQ ID NO: 39 - RNA, 20 nt, synthetic construct
  misc_feature 1..20: note = Synthetic
  source 1..20: mol_type = other RNA
aaagttgaacaatagaaaaa

SEQ ID NO: 40 - RNA, 20 nt, synthetic construct
  misc_feature 1..20: note = Synthetic
  source 1..20: mol_type = other RNA
aatgcataatctaagtcaaa

SEQ ID NO: 41 - RNA, 20 nt, synthetic construct
  misc_feature 1..20: note = Synthetic
  source 1..20: mol_type = other RNA
taataaaattcaaacatcct

SEQ ID NO: 42 - RNA, 20 nt, synthetic construct
  misc_feature 1..20: note = Synthetic
  source 1..20: mol_type = other RNA
gcatctttaaagaattattt

SEQ ID NO: 43 - RNA, 20 nt, synthetic construct
  misc_feature 1..20: note = Synthetic
  source 1..20: mol_type = other RNA
tttggcatttatttctaaaa

SEQ ID NO: 44 - RNA, 20 nt, synthetic construct
  misc_feature 1..20: note = Synthetic
  source 1..20: mol_type = other RNA
tgtatttgtgaagtcttaca

SEQ ID NO: 45 - RNA, 20 nt, synthetic construct
  misc_feature 1..20: note = Synthetic
  source 1..20: mol_type = other RNA
tcctaggtaaaaaaaaaaaa

SEQ ID NO: 46 - RNA, 20 nt, synthetic construct
  misc_feature 1..20: note = Synthetic
  source 1..20: mol_type = other RNA
taattttcttttgcgcacta

SEQ ID NO: 47 - RNA, 20 nt, synthetic construct
  misc_feature 1..20: note = Synthetic
  source 1..20: mol_type = other RNA
tgactgaaacttcacagaat

SEQ ID NO: 48 - RNA, 20 nt, synthetic construct
  misc_feature 1..20: note = Synthetic
  source 1..20: mol_type = other RNA
gactgaaacttcacagaata

SEQ ID NO: 49 - RNA, 20 nt, synthetic construct
  misc_feature 1..20: note = Synthetic
  source 1..20: mol_type = other RNA
ttcattttagtctgtcttct

SEQ ID NO: 50 - RNA, 20 nt, synthetic construct
  misc_feature 1..20: note = Synthetic
  source 1..20: mol_type = other RNA
attatctaagtttgaatata

SEQ ID NO: 51 - RNA, 20 nt, synthetic construct
  misc_feature 1..20: note = Synthetic
  source 1..20: mol_type = other RNA
aatttttaaaatagtattct

SEQ ID NO: 52 - RNA, 20 nt, synthetic construct
  misc_feature 1..20: note = Synthetic
  source 1..20: mol_type = other RNA
tgaattattcttctgtttaa

SEQ ID NO: 53 - RNA, 20 nt, synthetic construct
  misc_feature 1..20: note = Synthetic
  source 1..20: mol_type = other RNA
atcatcctgagtttttctgt

SEQ ID NO: 54 - RNA, 20 nt, synthetic construct
  misc_feature 1..20: note = Synthetic
  source 1..20: mol_type = other RNA
ttactaaaactttattttac

SEQ ID NO: 55 - RNA, 20 nt, synthetic construct
  misc_feature 1..20: note = Synthetic
  source 1..20: mol_type = other RNA
acctttttttttttttacct

SEQ ID NO: 56 - RNA, 20 nt, synthetic construct
  misc_feature 1..20: note = Synthetic
  source 1..20: mol_type = other RNA
agtgcaatggataggtcttt

SEQ ID NO: 57 - RNA, 20 nt, synthetic construct
  misc_feature 1..20: note = Synthetic
  source 1..20: mol_type = other RNA
tgattcctacagaaaaactc

SEQ ID NO: 58 - RNA, 20 nt, synthetic construct
  misc_feature 1..20: note = Synthetic
  source 1..20: mol_type = other RNA
tgggcaagggaagaaaaaaa

SEQ ID NO: 59 - RNA, 20 nt, synthetic construct
  misc_feature 1..20: note = Synthetic
  source 1..20: mol_type = other RNA
cctcactcttgtctgggcaa

SEQ ID NO: 60 - RNA, 20 nt, synthetic construct
  misc_feature 1..20: note = Synthetic
  source 1..20: mol_type = other RNA
acctcactcttgtctgggca

SEQ ID NO: 61 - RNA, 20 nt, synthetic construct
  misc_feature 1..20: note = Synthetic
  source 1..20: mol_type = other RNA
tgagcaacctcactcttgtc

SEQ ID NO: 62 - RNA, 100 nt, synthetic construct
  misc_feature 1..100: note = Synthetic
  source 1..100: mol_type = other RNA
gagcaacctcactcttgtctgttttagagctagaaatagcaagttaaaataaggctagtccgttatcaacttgaaaaagtggcaccgagtcggtgctttt

SEQ ID NO: 63 - RNA, 100 nt, synthetic construct
  misc_feature 1..100: note = Synthetic
  source 1..100: mol_type = other RNA
atgcatttgtttcaaaatatgttttagagctagaaatagcaagttaaaataaggctagtccgttatcaacttgaaaaagtggcaccgagtcggtgctttt

SEQ ID NO: 64 - RNA, 100 nt, synthetic construct
  misc_feature 1..100: note = Synthetic
  source 1..100: mol_type = other RNA
tgcatttgtttcaaaatattgttttagagctagaaatagcaagttaaaataaggctagtccgttatcaacttgaaaaagtggcaccgagtcggtgctttt

SEQ ID NO: 65 - RNA, 100 nt, synthetic construct
  misc_feature 1..100: note = Synthetic
  source 1..100: mol_type = other RNA
atttatgagatcaacagcacgttttagagctagaaatagcaagttaaaataaggctagtccgttatcaacttgaaaaagtggcaccgagtcggtgctttt

SEQ ID NO: 66 - RNA, 100 nt, synthetic construct
  misc_feature 1..100: note = Synthetic
  source 1..100: mol_type = other RNA
gatcaacagcacaggttttggttttagagctagaaatagcaagttaaaataaggctagtccgttatcaacttgaaaaagtggcaccgagtcggtgctttt

SEQ ID NO: 67 - RNA, 100 nt, synthetic construct
  misc_feature 1..100: note = Synthetic
  source 1..100: mol_type = other RNA
ttaaataaagcatagtgcaagttttagagctagaaatagcaagttaaaataaggctagtccgttatcaacttgaaaaagtggcaccgagtcggtgctttt

SEQ ID NO: 68 - RNA, 100 nt, synthetic construct
  misc_feature 1..100: note = Synthetic
  source 1..100: mol_type = other RNA
taaagcatagtgcaatggatgttttagagctagaaatagcaagttaaaataaggctagtccgttatcaacttgaaaaagtggcaccgagtcggtgctttt

SEQ ID NO: 69 - RNA, 100 nt, synthetic construct
  misc_feature 1..100: note = Synthetic
  source 1..100: mol_type = other RNA
tagtgcaatggataggtcttgttttagagctagaaatagcaagttaaaataaggctagtccgttatcaacttgaaaaagtggcaccgagtcggtgctttt

SEQ ID NO: 70 - RNA, 100 nt, synthetic construct
  misc_feature 1..100: note = Synthetic
  source 1..100: mol_type = other RNA
tactaaaactttattttactgttttagagctagaaatagcaagttaaaataaggctagtccgttatcaacttgaaaaagtggcaccgagtcggtgctttt

SEQ ID NO: 71 - RNA, 100 nt, synthetic construct
  misc_feature 1..100: note = Synthetic
  source 1..100: mol_type = other RNA
aaagttgaacaatagaaaaagttttagagctagaaatagcaagttaaaataaggctagtccgttatcaacttgaaaaagtggcaccgagtcggtgctttt

SEQ ID NO: 72 - RNA, 100 nt, synthetic construct
  misc_feature 1..100: note = Synthetic
  source 1..100: mol_type = other RNA
aatgcataatctaagtcaaagttttagagctagaaatagcaagttaaaataaggctagtccgttatcaacttgaaaaagtggcaccgagtcggtgctttt

SEQ ID NO: 73 - RNA, 100 nt, synthetic construct
  misc_feature 1..100: note = Synthetic
  source 1..100: mol_type = other RNA
taataaaattcaaacatcctgttttagagctagaaatagcaagttaaaataaggctagtccgttatcaacttgaaaaagtggcaccgagtcggtgctttt

SEQ ID NO: 74 - RNA, 100 nt, synthetic construct
  misc_feature 1..100: note = Synthetic
  source 1..100: mol_type = other RNA
gcatctttaaagaattatttgttttagagctagaaatagcaagttaaaataaggctagtccgttatcaacttgaaaaagtggcaccgagtcggtgctttt

SEQ ID NO: 75 - RNA, 100 nt, synthetic construct
  misc_feature 1..100: note = Synthetic
  source 1..100: mol_type = other RNA
tttggcatttatttctaaaagttttagagctagaaatagcaagttaaaataaggctagtccgttatcaacttgaaaaagtggcaccgagtcggtgctttt

SEQ ID NO: 76 - RNA, 100 nt, synthetic construct
  misc_feature 1..100: note = Synthetic
  source 1..100: mol_type = other RNA
tgtatttgtgaagtcttacagttttagagctagaaatagcaagttaaaataaggctagtccgttatcaacttgaaaaagtggcaccgagtcggtgctttt

SEQ ID NO: 77 - RNA, 100 nt, synthetic construct
  misc_feature 1..100: note = Synthetic
  source 1..100: mol_type = other RNA
tcctaggtaaaaaaaaaaaagttttagagctagaaatagcaagttaaaataaggctagtccgttatcaacttgaaaaagtggcaccgagtcggtgctttt

SEQ ID NO: 78 - RNA, 100 nt, synthetic construct
  misc_feature 1..100: note = Synthetic
  source 1..100: mol_type = other RNA
taattttcttttgcgcactagttttagagctagaaatagcaagttaaaataaggctagtccgttatcaacttgaaaaagtggcaccgagtcggtgctttt

SEQ ID NO: 79 - RNA, 100 nt, synthetic construct
  misc_feature 1..100: note = Synthetic
  source 1..100: mol_type = other RNA
tgactgaaacttcacagaatgttttagagctagaaatagcaagttaaaataaggctagtccgttatcaacttgaaaaagtggcaccgagtcggtgctttt

SEQ ID NO: 80 - RNA, 100 nt, synthetic construct
  misc_feature 1..100: note = Synthetic
  source 1..100: mol_type = other RNA
gactgaaacttcacagaatagttttagagctagaaatagcaagttaaaataaggctagtccgttatcaacttgaaaaagtggcaccgagtcggtgctttt

SEQ ID NO: 81 - RNA, 100 nt, synthetic construct
  misc_feature 1..100: note = Synthetic
  source 1..100: mol_type = other RNA
ttcattttagtctgtcttctgttttagagctagaaatagcaagttaaaataaggctagtccgttatcaacttgaaaaagtggcaccgagtcggtgctttt

SEQ ID NO: 82 - RNA, 100 nt, synthetic construct
  misc_feature 1..100: note = Synthetic
  source 1..100: mol_type = other RNA
attatctaagtttgaatatagttttagagctagaaatagcaagttaaaataaggctagtccgttatcaacttgaaaaagtggcaccgagtcggtgctttt

SEQ ID NO: 83 - RNA, 100 nt, synthetic construct
  misc_feature 1..100: note = Synthetic
  source 1..100: mol_type = other RNA
aatttttaaaatagtattctgttttagagctagaaatagcaagttaaaataaggctagtccgttatcaacttgaaaaagtggcaccgagtcggtgctttt

SEQ ID NO: 84 - RNA, 100 nt, synthetic construct
  misc_feature 1..100: note = Synthetic
  source 1..100: mol_type = other RNA
tgaattattcttctgtttaagttttagagctagaaatagcaagttaaaataaggctagtccgttatcaacttgaaaaagtggcaccgagtcggtgctttt

SEQ ID NO: 85 - RNA, 100 nt, synthetic construct
  misc_feature 1..100: note = Synthetic
  source 1..100: mol_type = other RNA
atcatcctgagtttttctgtgttttagagctagaaatagcaagttaaaataaggctagtccgttatcaacttgaaaaagtggcaccgagtcggtgctttt

SEQ ID NO: 86 - RNA, 100 nt, synthetic construct
  misc_feature 1..100: note = Synthetic
  source 1..100: mol_type = other RNA
ttactaaaactttattttacgttttagagctagaaatagcaagttaaaataaggctagtccgttatcaacttgaaaaagtggcaccgagtcggtgctttt

SEQ ID NO: 87 - RNA, 100 nt, synthetic construct
  misc_feature 1..100: note = Synthetic
  source 1..100: mol_type = other RNA
acctttttttttttttacctgttttagagctagaaatagcaagttaaaataaggctagtccgttatcaacttgaaaaagtggcaccgagtcggtgctttt

SEQ ID NO: 88 - RNA, 100 nt, synthetic construct
  misc_feature 1..100: note = Synthetic
  source 1..100: mol_type = other RNA
agtgcaatggataggtctttgttttagagctagaaatagcaagttaaaataaggctagtccgttatcaacttgaaaaagtggcaccgagtcggtgctttt

SEQ ID NO: 89 - RNA, 100 nt, synthetic construct
  misc_feature 1..100: note = Synthetic
  source 1..100: mol_type = other RNA
tgattcctacagaaaaactcgttttagagctagaaatagcaagttaaaataaggctagtccgttatcaacttgaaaaagtggcaccgagtcggtgctttt

SEQ ID NO: 90 - RNA, 100 nt, synthetic construct
  misc_feature 1..100: note = Synthetic
  source 1..100: mol_type = other RNA
tgggcaagggaagaaaaaaagttttagagctagaaatagcaagttaaaataaggctagtccgttatcaacttgaaaaagtggcaccgagtcggtgctttt

SEQ ID NO: 91 - RNA, 100 nt, synthetic construct
  misc_feature 1..100: note = Synthetic
  source 1..100: mol_type = other RNA
cctcactcttgtctgggcaagttttagagctagaaatagcaagttaaaataaggctagtccgttatcaacttgaaaaagtggcaccgagtcggtgctttt

SEQ ID NO: 92 - RNA, 100 nt, synthetic construct
  misc_feature 1..100: note = Synthetic
  source 1..100: mol_type = other RNA
acctcactcttgtctgggcagttttagagctagaaatagcaagttaaaataaggctagtccgttatcaacttgaaaaagtggcaccgagtcggtgctttt

SEQ ID NO: 93 - RNA, 100 nt, synthetic construct
  misc_feature 1..100: note = Synthetic
  source 1..100: mol_type = other RNA
tgagcaacctcactcttgtcgttttagagctagaaatagcaagttaaaataaggctagtccgttatcaacttgaaaaagtggcaccgagtcggtgctttt

SEQ ID NO: 94 - RNA, 100 nt, synthetic construct; identical to SEQ ID NO: 62
  misc_feature 1..100: note = Synthetic
  source 1..100: mol_type = other RNA

SEQ ID NO: 95 - RNA, 100 nt, synthetic construct; identical to SEQ ID NO: 63
  misc_feature 1..100: note = Synthetic
  source 1..100: mol_type = other RNA

SEQ ID NO: 96 - RNA, 100 nt, synthetic construct; identical to SEQ ID NO: 64
  misc_feature 1..100: note = Synthetic
  source 1..100: mol_type = other RNA

SEQ ID NO: 97 - RNA, 100 nt, synthetic construct; identical to SEQ ID NO: 65
  misc_feature 1..100: note = Synthetic
  source 1..100: mol_type = other RNA

SEQ ID NO: 98 - RNA, 100 nt, synthetic construct; identical to SEQ ID NO: 66
  misc_feature 1..100: note = Synthetic
  source 1..100: mol_type = other RNA

SEQ ID NO: 99 - RNA, 100 nt, synthetic construct; identical to SEQ ID NO: 67
  misc_feature 1..100: note = Synthetic
  source 1..100: mol_type = other RNA

SEQ ID NO: 100 - RNA, 100 nt, synthetic construct; identical to SEQ ID NO: 68
  misc_feature 1..100: note = Synthetic
  source 1..100: mol_type = other RNA

SEQ ID NO: 101 - RNA, 100 nt, synthetic construct; identical to SEQ ID NO: 69
  misc_feature 1..100: note = Synthetic
  source 1..100: mol_type = other RNA

SEQ ID NO: 102 - RNA, 100 nt, synthetic construct; identical to SEQ ID NO: 70
  misc_feature 1..100: note = Synthetic
  source 1..100: mol_type = other RNA

SEQ ID NO: 103 - RNA, 100 nt, synthetic construct; identical to SEQ ID NO: 71
  misc_feature 1..100: note = Synthetic
  source 1..100: mol_type = other RNA

SEQ ID NO: 104 - RNA, 100 nt, synthetic construct; identical to SEQ ID NO: 72
  misc_feature 1..100: note = Synthetic
  source 1..100: mol_type = other RNA

SEQ ID NO: 105 - RNA, 100 nt, synthetic construct; identical to SEQ ID NO: 73
  misc_feature 1..100: note = Synthetic
  source 1..100: mol_type = other RNA

SEQ ID NO: 106 - RNA, 100 nt, synthetic construct; identical to SEQ ID NO: 74
  misc_feature 1..100: note = Synthetic
  source 1..100: mol_type = other RNA

SEQ ID NO: 107 - RNA, 100 nt, synthetic construct; identical to SEQ ID NO: 75
  misc_feature 1..100: note = Synthetic
  source 1..100: mol_type = other RNA

SEQ ID NO: 108 - RNA, 100 nt, synthetic construct; identical to SEQ ID NO: 76
  misc_feature 1..100: note = Synthetic
  source 1..100: mol_type = other RNA

SEQ ID NO: 109 - RNA, 100 nt, synthetic construct; identical to SEQ ID NO: 77
  misc_feature 1..100: note = Synthetic
  source 1..100: mol_type = other RNA

SEQ ID NO: 110 - RNA, 100 nt, synthetic construct; identical to SEQ ID NO: 78
  misc_feature 1..100: note = Synthetic
  source 1..100: mol_type = other RNA

SEQ ID NO: 111 - RNA, 100 nt, synthetic construct; identical to SEQ ID NO: 79
  misc_feature 1..100: note = Synthetic
  source 1..100: mol_type = other RNA

SEQ ID NO: 112 - RNA, 100 nt, synthetic construct; identical to SEQ ID NO: 80
  misc_feature 1..100: note = Synthetic
  source 1..100: mol_type = other RNA

SEQ ID NO: 113 - RNA, 100 nt, synthetic construct; identical to SEQ ID NO: 81
  misc_feature 1..100: note = Synthetic
  source 1..100: mol_type = other RNA

SEQ ID NO: 114 - RNA, 100 nt, synthetic construct; identical to SEQ ID NO: 82
  misc_feature 1..100: note = Synthetic
  source 1..100: mol_type = other RNA

SEQ ID NO: 115 - RNA, 100 nt, synthetic construct; identical to SEQ ID NO: 83
  misc_feature 1..100: note = Synthetic
  source 1..100: mol_type = other RNA

SEQ ID NO: 116 - RNA, 100 nt, synthetic construct; identical to SEQ ID NO: 84
  misc_feature 1..100: note = Synthetic
  source 1..100: mol_type = other RNA

SEQ ID NO: 117 - RNA, 100 nt, synthetic construct; identical to SEQ ID NO: 85
  misc_feature 1..100: note = Synthetic
  source 1..100: mol_type = other RNA

SEQ ID NO: 118 - RNA, 100 nt, synthetic construct; identical to SEQ ID NO: 86
  misc_feature 1..100: note = Synthetic
  source 1..100: mol_type = other RNA

SEQ ID NO: 119 - RNA, 100 nt, synthetic construct; identical to SEQ ID NO: 87
  misc_feature 1..100: note = Synthetic
  source 1..100: mol_type = other RNA

SEQ ID NO: 120 - RNA, 100 nt, synthetic construct; identical to SEQ ID NO: 88
  misc_feature 1..100: note = Synthetic
  source 1..100: mol_type = other RNA

SEQ ID NO: 121 - RNA, 100 nt, synthetic construct; identical to SEQ ID NO: 89
  misc_feature 1..100: note = Synthetic
  source 1..100: mol_type = other RNA

SEQ ID NO: 122 - RNA, 100 nt, synthetic construct; identical to SEQ ID NO: 90
  misc_feature 1..100: note = Synthetic
  source 1..100: mol_type = other RNA

SEQ ID NO: 123 - RNA, 100 nt, synthetic construct; identical to SEQ ID NO: 91
  misc_feature 1..100: note = Synthetic
  source 1..100: mol_type = other RNA

SEQ ID NO: 124 - RNA, 100 nt, synthetic construct; identical to SEQ ID NO: 92
  misc_feature 1..100: note = Synthetic
  source 1..100: mol_type = other RNA

SEQ ID NO: 125 - RNA, 100 nt, synthetic construct; identical to SEQ ID NO: 93
  misc_feature 1..100: note = Synthetic
  source 1..100: mol_type = other RNA

SEQ ID NO: 126 - DNA, 20 bp, synthetic construct
  misc_feature 1..20: note = Synthetic
  source 1..20: mol_type = other DNA
gagcaacctcactcttgtct

SEQ ID NO: 127 - DNA, 20 bp, synthetic construct
  misc_feature 1..20: note = Synthetic
  source 1..20: mol_type = other DNA
atgcatttgtttcaaaatat

SEQ ID NO: 128 - DNA, 20 bp, synthetic construct
  misc_feature 1..20: note = Synthetic
  source 1..20: mol_type = other DNA
tgcatttgtttcaaaatatt

SEQ ID NO: 129 - DNA, 20 bp, synthetic construct
  misc_feature 1..20: note = Synthetic
  source 1..20: mol_type = other DNA
atttatgagatcaacagcac

SEQ ID NO: 130 - DNA, 20 bp, synthetic construct
  misc_feature 1..20: note = Synthetic
  source 1..20: mol_type = other DNA
gatcaacagcacaggttttg

SEQ ID NO: 131 - DNA, 20 bp, synthetic construct
  misc_feature 1..20: note = Synthetic
  source 1..20: mol_type = other DNA
ttaaataaagcatagtgcaa

SEQ ID NO: 132 - DNA, 20 bp, synthetic construct
  misc_feature 1..20: note = Synthetic
  source 1..20: mol_type = other DNA
taaagcatagtgcaatggat

SEQ ID NO: 133 - DNA, 20 bp, synthetic construct
  misc_feature 1..20: note = Synthetic
  source 1..20: mol_type = other DNA
tagtgcaatggataggtctt

SEQ ID NO: 134 - DNA, 20 bp, synthetic construct
  misc_feature 1..20: note = Synthetic
  source 1..20: mol_type = other DNA
tactaaaactttattttact

SEQ ID NO: 135 - DNA, 20 bp, synthetic construct
  misc_feature 1..20: note = Synthetic
  source 1..20: mol_type = other DNA
aaagttgaacaatagaaaaa

SEQ ID NO: 136 - DNA, 20 bp, synthetic construct
  misc_feature 1..20: note = Synthetic
  source 1..20: mol_type = other DNA
aatgcataatctaagtcaaa

SEQ ID NO: 137 - DNA, 20 bp, synthetic construct
  misc_feature 1..20: note = Synthetic
  source 1..20: mol_type = other DNA
taataaaattcaaacatcct

SEQ ID NO: 138 - DNA, 20 bp, synthetic construct
  misc_feature 1..20: note = Synthetic
  source 1..20: mol_type = other DNA
gcatctttaaagaattattt

SEQ ID NO: 139 - DNA, 20 bp, synthetic construct
  misc_feature 1..20: note = Synthetic
  source 1..20: mol_type = other DNA
tttggcatttatttctaaaa

SEQ ID NO: 140 - DNA, 20 bp, synthetic construct
  misc_feature 1..20: note = Synthetic
  source 1..20: mol_type = other DNA
tgtatttgtgaagtcttaca

SEQ ID NO: 141 - DNA, 20 bp, synthetic construct
  misc_feature 1..20: note = Synthetic
  source 1..20: mol_type = other DNA
tcctaggtaaaaaaaaaaaa

SEQ ID NO: 142 - DNA, 20 bp, synthetic construct
  misc_feature 1..20: note = Synthetic
  source 1..20: mol_type = other DNA
taattttcttttgcgcacta

SEQ ID NO: 143 - DNA, 20 bp, synthetic construct
  misc_feature 1..20: note = Synthetic
  source 1..20: mol_type = other DNA
tgactgaaacttcacagaat

SEQ ID NO: 144 - DNA, 20 bp, synthetic construct
  misc_feature 1..20: note = Synthetic
  source 1..20: mol_type = other DNA
gactgaaacttcacagaata

SEQ ID NO: 145 - DNA, 20 bp, synthetic construct
  misc_feature 1..20: note = Synthetic
  source 1..20: mol_type = other DNA
ttcattttagtctgtcttct

SEQ ID NO: 146 - DNA, 20 bp, synthetic construct
  misc_feature 1..20: note = Synthetic
  source 1..20: mol_type = other DNA
attatctaagtttgaatata

SEQ ID NO: 147 - DNA, 20 bp, synthetic construct
  misc_feature 1..20: note = Synthetic
  source 1..20: mol_type = other DNA
aatttttaaaatagtattct

SEQ ID NO: 148 - DNA, 20 bp, synthetic construct
  misc_feature 1..20: note = Synthetic
  source 1..20: mol_type = other DNA
tgaattattcttctgtttaa

SEQ ID NO: 149 - DNA, 20 bp, synthetic construct
  misc_feature 1..20: note = Synthetic
  source 1..20: mol_type = other DNA
atcatcctgagtttttctgt

SEQ ID NO: 150 - DNA, 20 bp, synthetic construct
  misc_feature 1..20: note = Synthetic
  source 1..20: mol_type = other DNA
ttactaaaactttattttac

SEQ ID NO: 151 - DNA, 20 bp, synthetic construct
  misc_feature 1..20: note = Synthetic
  source 1..20: mol_type = other DNA
acctttttttttttttacct

SEQ ID NO: 152 - DNA, 20 bp, synthetic construct
  misc_feature 1..20: note = Synthetic
  source 1..20: mol_type = other DNA
agtgcaatggataggtcttt

SEQ ID NO: 153 - DNA, 20 bp, synthetic construct
  misc_feature 1..20: note = Synthetic
  source 1..20: mol_type = other DNA
tgattcctacagaaaaactc

SEQ ID NO: 154 - DNA, 20 bp, synthetic construct
  misc_feature 1..20: note = Synthetic
  source 1..20: mol_type = other DNA
tgggcaagggaagaaaaaaa

SEQ ID NO: 155 - DNA, 20 bp, synthetic construct
  misc_feature 1..20: note = Synthetic
  source 1..20: mol_type = other DNA
cctcactcttgtctgggcaa

SEQ ID NO: 156 - DNA, 20 bp, synthetic construct
  misc_feature 1..20: note = Synthetic
  source 1..20: mol_type = other DNA
acctcactcttgtctgggca

SEQ ID NO: 157 - DNA, 20 bp, synthetic construct
  misc_feature 1..20: note = Synthetic
  source 1..20: mol_type = other DNA
tgagcaacctcactcttgtc

SEQ ID NO: 158 - DNA, 1296 bp, synthetic construct
  misc_feature 1..1296: note = Synthetic
  source 1..1296: mol_type = other DNA
tttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaa

SEQ ID NO: 159 - DNA, 1296 bp, synthetic construct
  misc_feature 1..1296: note = Synthetic
  misc_feature 60: note = Cryptic Splice Donor Site Mutated
  misc_feature 201: note = Cryptic Splice Donor Site Mutated
  misc_feature 618: note = Cryptic Splice Donor Site Mutated
  source 1..1296: mol_type = other DNA
tttcttgatcatgaaaatgccaacaaaattctgaataggccaaagaggtataattcagggaaattggaagagtttgttcaagggaaccttgagagagaatgtatggaagaaaagtgtagttttgaagaagcaagagaagtttttgaaaacactgaaagaacaactgaattttggaagcagtatgttgatggagatcagtgcgagtccaatccatgtttaaatgggggcagttgcaaggatgacattaattcctatgaatgttggtgtccctttggatttgaaggaaagaactgtgaattagatgtaacatgtaacattaagaatggcagatgtgagcagttttgtaaaaatagtgctgataacaaggtggtttgctcctgtactgagggatatagacttgcagaaaaccagaagtcctgtgaaccagcagtgccatttccatgtggaagagtttctgtttcacaaacttctaagctcaccagagctgagactgtttttcctgatgtggactatgtaaattctactgaagctgaaaccattttggataacatcactcaaagcacccaatcatttaatgacttcactagggttgttggtggagaagatgccaaaccaggacaattcccttggcaggttgttttgaatggtaaagttgatgcattctgtggaggctctattgttaatgaaaaatggattgtaactgctgcccactgtgttgaaactggtgttaaaattacagttgtggcaggtgaacataatattgaggagacagaacatacagagcaaaagagaaatgtgattagaattattcctcaccacaactacaatgcagctattaataagtacaaccatgacattgcccttctggaactggatgaacccttagtgctaaacagctatgttacacctatttgcattgctgacaaggaatacaccaacatcttcctcaaatttggatctggctatgtaagtggctggggaagagtcttccacaaagggagatcagctttagttcttcagtaccttagagttccacttgttgacagagccacatgtcttaggtctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatccaggtatgtcaactggattaaggaaaaaacaaagctcacttaa

SEQ ID NO: 160 - DNA, 1296 bp, synthetic construct
  misc_feature 1..1296: note = Synthetic
  source 1..1296: mol_type = other DNA
tttcttgatcatgaaaatgccaacaaaattctgaataggccaaagaggtataattcaggtaaattggaagagtttgttcaagggaaccttgagagagaatgtatggaagaaaagtgtagttttgaagaagcaagagaagtttttgaaaacactgaaagaacaactgaattttggaagcagtatgttgatggagatcagtgtgagtccaatccatgtttaaatgggggcagttgcaaggatgacattaattcctatgaatgttggtgtccctttggatttgaaggaaagaactgtgaattagatgtaacatgtaacattaagaatggcagatgtgagcagttttgtaaaaatagtgctgataacaaggtggtttgctcctgtactgagggatatagacttgcagaaaaccagaagtcctgtgaaccagcagtgccatttccatgtggaagagtttctgtttcacaaacttctaagctcaccagagctgagactgtttttcctgatgtggactatgtaaattctactgaagctgaaaccattttggataacatcactcaaagcacccaatcatttaatgacttcactagggttgttggtggagaagatgccaaaccaggtcaattcccttggcaggttgttttgaatggtaaagttgatgcattctgtggaggctctattgttaatgaaaaatggattgtaactgctgcccactgtgttgaaactggtgttaaaattacagttgtggcaggtgaacataatattgaggagacagaacatacagagcaaaagagaaatgtgattagaattattcctcaccacaactacaatgcagctattaataagtacaaccatgacattgcccttctggaactggatgaacccttagtgctaaacagctatgttacacctatttgcattgctgacaaggaatacaccaacatcttcctcaaatttggatctggctatgtaagtggctggggaagagtcttccacaaagggagatcagctttagttcttcagtaccttagagttccacttgttgacagagccacatgtcttaggtctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatccaggtatgtcaactggattaaggaaaaaacaaagctcacttaa

SEQ ID NO: 161 - DNA, 1296 bp, synthetic construct
  misc_feature 1..1296: note = Synthetic
  source 1..1296: mol_type = other DNA
tttcttgatcatgaaaacgccaacaaaattctgaatcggccaaagaggtataattcagggaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgcgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggacaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaa

SEQ ID NO: 162 - DNA, 1296 bp, synthetic construct
  misc_feature 1..1296: note = Synthetic
  source 1..1296: mol_type = other DNA
tttctggaccacgaaaacgcgaataagatacttaaccggcctaaaaggtataactcaggaaagcttgaagaattcgtgcaagggaacttggagcgagagtgtatggaggaaaaatgcagttttgaagaagcgcgcgaggtgtttgagaatacagaacggaccactgagttctggaagcaatatgtcgatggggaccaatgcgaatctaatccatgtctgaatggggggagttgtaaagacgatataaattcctacgagtgttggtgcccttttggttttgaaggaaaaaactgcgagttggacgtcacctgcaacattaaaaatggacgatgcgagcaattctgtaaaaattccgccgacaacaaagttgtgtgtagttgcactgagggctaccggctcgctgagaatcaaaagagctgtgaaccggcggtgcccttcccgtgcggtcgggtaagtgtgtcccagacatcaaagttgacaagggccgagacagtttttcccgatgtggactacgttaactctactgaagccgaaacgattcttgataatataacacaatccacacagtcatttaatgactttactagggttgtcgggggcgaggacgctaaacctggccaatttccatggcaggtggtgctcaacggaaaagtcgacgcgttttgtgggggctccatagtcaatgaaaagtggattgtaacggccgcacactgtgtcgagacgggggttaagattacggtcgtggctggcgaacacaacattgaagaaactgagcatactgaacagaaaaggaatgttatcaggatcataccccatcacaattataatgccgctataaacaagtacaaccatgatatagccctcctggagctggacgagccactcgtacttaactcctatgttaccccgatttgtatagccgataaagaatatacaaatatcttcttgaaatttgggagcggatatgtgagtgggtgggggcgggtcttccacaaaggtcgatcagccctcgttctgcaatatttgcgcgtcccgttggtcgatagagcgacctgtcttcggtccacgaaatttacgatttacaataacatgttttgtgctgggtttcacgagggcggtcgcgactcatgccaaggtgattcaggtggaccacacgtcactgaagtcgaaggaacaagtttcttgaccgggataataagttggggggaggaatgtgcgatgaaggggaaatatggcatctatacgaaggtctctcgctacgtgaattggataaaagaaaagactaagctcacctaa

SEQ ID NO: 163 - DNA, 1296 bp, synthetic construct
  misc_feature 1..1296: note = Synthetic
  source 1..1296: mol_type = other DNA
tttcttgatcatgaaaatgccaacaaaattctgaatagaccaaagaggtataactctggcaagcttgaagagtttgtacaggggaatctggagagagagtgtatggaagagaagtgcagctttgaggaagccagagaagtgtttgaaaatacagagagaacaactgaattttggaagcagtatgtggatggtgatcaatgtgagagcaatccctgcttgaatggggggagctgtaaagatgatatcaacagctatgaatgttggtgtccctttggatttgaggggaaaaactgtgagcttgatgtgacctgtaatatcaagaatggcaggtgtgagcaattttgcaagaattctgctgataacaaagtggtctgtagctgcactgagggatataggctggctgaaaaccagaagagctgtgaacctgcagtgccttttccctgtgggagagtgtctgtgagccaaaccagcaagctgactagggctgaaacagtctttcctgatgtagattatgtgaatagcactgaggctgagacaatccttgacaatatcactcagagcacacagagcttcaatgacttcaccagggtggtaggaggggaggatgccaagcctgggcagttcccctggcaggtagtgctcaatggaaaagtggatgccttttgtggaggttcaattgtaaatgagaagtggattgtgactgcagcccactgtgtggaaactggagtcaagattactgtggtggctggagagcacaatattgaggaaactgagcacactgagcagaagaggaatgtgatcaggattatcccccaccacaactacaatgctgctatcaacaagtacaaccatgacattgccctcctggaactggatgaacccctggtcttgaacagctatgtgacacccatctgtattgctgataaagagtacaccaacatcttcttgaaatttgggtctggatatgtgtctggctggggcagggtgttccataaaggcaggtctgccctggtattgcagtatttgagggtgcctctggtggatagagcaacctgcttgaggagcaccaagtttacaatctacaacaatatgttctgtgcagggttccatgaaggtggtagagacagctgccagggagattctgggggtccccatgtgactgaggtggagggaaccagcttcctgactgggattatcagctggggtgaggagtgtgctatgaagggaaagtatgggatctacacaaaagtatccagatatgtgaactggattaaggagaaaaccaagctgacttga

SEQ ID NO: 164 - DNA, 1296 bp, synthetic construct
  misc_feature 1..1296: note = Synthetic
  source 1..1296: mol_type = other DNA
ttcctggaccatgagaatgccaacaagatcctgaacaggcccaagaggtacaactctggcaagctggaggagtttgtgcagggcaacctggagagagagtgcatggaggagaagtgcagctttgaggaggccagggaggtgtttgagaacacagagagaaccacagagttctggaagcagtatgtggatggagaccagtgtgagagcaacccctgcctgaatggaggcagctgcaaggatgacatcaacagctatgagtgctggtgcccctttggctttgagggcaagaactgtgagctggatgtgacctgcaacatcaagaatggcagatgtgagcagttctgcaagaactctgctgacaacaaggtggtgtgcagctgcacagagggctacaggctggctgagaaccagaagagctgtgagcctgctgtgcccttcccctgtggcagagtgtctgtgagccagaccagcaagctgaccagggctgagacagtgttccctgatgtggactatgtgaacagcacagaggctgagaccatcctggacaacatcacccagagcacccagagcttcaatgacttcaccagggtggtggggggagaggatgccaagcctggccagttcccctggcaggtggtgctgaatggcaaggtggatgccttctgtggaggcagcattgtgaatgagaagtggattgtgacagctgcccactgtgtggagacaggggtgaagatcacagtggtggctggggagcacaacattgaggagacagagcacacagagcagaagaggaatgtgatcaggatcatcccccaccacaactacaatgctgccatcaacaagtacaaccatgacattgccctgctggagctggatgagcccctggtgctgaacagctatgtgacccccatctgcattgctgacaaggagtacaccaacatcttcctgaagtttggctctggctatgtgtctggctggggcagagtgttccacaagggcaggtctgccctggtgctgcagtacctgagagtgcccctggtggacagggccacctgcctgaggagcaccaagttcaccatctacaacaacatgttctgtgctggcttccatgagggaggcagagacagctgccagggagactctgggggcccccatgtgacagaggtggagggcaccagcttcctgacaggcatcatcagctggggggaggagtgtgccatgaagggcaagtatggcatctacaccaaggtgagcaggtatgtgaactggatcaaggagaagaccaagctgacctga

SEQ ID NO: 165 - DNA, 1296 bp, synthetic construct
  misc_feature 1..1296: note = Synthetic
  source 1..1296: mol_type = other DNA
ttcctggaccatgagaatgccaacaagatcctgaacaggcccaagaggtacaactctggcaagctggaagagtttgtgcagggcaacctggaaagggaatgcatggaagagaagtgcagctttgaagaggccagggaagtgtttgagaacacagagagaaccacagagttctggaagcagtatgtggatggggaccagtgtgaaagcaacccctgcctgaatggtggcagctgcaaggatgacatcaacagctatgagtgctggtgcccctttggctttgagggcaagaactgtgaactggatgtgacctgcaacatcaagaatggcagatgtgaacagttctgcaagaactctgctgacaacaaggttgtgtgctcctgcacagagggctacagactggctgagaaccagaaaagctgtgaacctgctgtgccctttccatgtggcagagtgtctgtgtcccagaccagcaagctgaccagagctgagacagtgttccctgatgtggactatgtgaactccacagaggctgaaaccatcctggacaacatcacccagagcacccagtccttcaatgacttcacaagagttgttggaggggaagatgccaagcctggacagttcccttggcaagtggtgctgaatggcaaagtggatgccttctgtggtggctccattgtgaatgagaagtggattgtgacagctgcccactgtgtggaaacaggggtcaagatcacagtggtggctggggagcacaacattgaggaaacagagcacacagagcaaaagaggaatgtcatcaggatcatccctcaccacaactacaatgctgccatcaacaagtacaaccatgacattgccctgcttgagctggatgagcccctggtcctgaactcctatgtgacccctatctgcattgctgacaaagagtacaccaacatctttctgaagtttggctctggctatgtgtctggctggggtagagtgttccacaagggaaggtctgccctggtgctgcagtacctgagagtgcccctggtggatagagccacctgtctgagaagcaccaagttcaccatctacaacaacatgttctgtgctgggttccatgaaggtggcagagactcctgccagggagatagtggtggccctcatgtgacagaggtggaaggcaccagctttctgacaggcatcatcagctggggagaagagtgtgccatgaagggcaaatatggcatctacaccaaggtgtccagatatgtcaactggatcaaagaaaagaccaagctcacctga

SEQ ID NO: 166 - DNA, 1296 bp, synthetic construct
  misc_feature 1..1296: note = Synthetic
  source 1..1296: mol_type = other DNA
ttcctggaccatgagaatgccaacaagatcctcaacagacccaagagatacaacagtggcaagctggaggagtttgtgcagggcaacctggagagggagtgcatggaagagaagtgcagctttgaggaagccagagaagtgtttgaaaacacagagagaaccactgagttctggaagcagtatgttgatggagaccagtgtgagagcaacccttgcctgaatggaggcagctgcaaagatgacatcaacagctatgagtgctggtgcccttttggctttgagggcaagaactgtgagctggatgtcacctgcaacatcaagaatggcagatgtgaacagttctgcaagaactcagcagacaacaaggtggtgtgcagctgcacagagggctacaggctggctgaaaaccagaagagctgtgaacctgctgtccctttcccctgtggcagagtgtctgtgtcccagacctccaagctgaccagggctgaaacagtgttccctgatgtggactatgtgaacagcactgaggctgagaccatcctggacaacatcacccagagcacccagagcttcaatgacttcaccagagtggtgggaggagaggatgccaagcctggccagttcccatggcaggtggtgctgaatggcaaggtggatgccttctgtgggggcagcattgtgaatgaaaaatggattgtgacagctgcccactgtgtggaaacaggtgtcaagatcacagtggtggctggtgaacacaacatagaagaaacagagcacacagagcagaagagaaatgtgatcaggatcatccctcaccacaactacaatgcagccatcaacaagtacaaccatgacattgccctgctggaacttgatgagcctctggtgctgaacagctatgttacccccatctgcattgcagacaaagagtacaccaacatcttcctgaagtttggctctggctatgtgtctggctggggcagagtgttccacaagggcaggtctgccctggtgctgcaatacctgagagtccccctggtggacagagccacctgcctgagaagcaccaagttcaccatctacaacaacatgttctgtgctggcttccatgagggaggcagagacagctgccaaggagactctggaggcccacatgtgacagaggtggagggcaccagcttcctgacaggcatcatcagctggggagaggaatgtgccatgaagggcaagtatggcatctacaccaaggtgtccagatatgtgaactggatcaaagaaaaaaccaaactgacctga

SEQ ID NO: 167 - DNA, 1296 bp, synthetic construct
  misc_feature 1..1296: note = Synthetic
  source 1..1296: mol_type = other DNA
ttcctggaccatgagaatgccaacaagatcctcaacagacccaagagatacaacagtggcaagctggaggagtttgtgcagggcaacctggagagggagtgcatggaagagaagtgcagctttgaggaagccagagaagtgtttgaaaacacagagagaaccactgagttctggaagcagtacgttgatggagaccagtgtgagagcaacccttgcctgaatggaggcagctgcaaagatgacatcaacagctatgagtgctggtgcccttttggctttgagggcaagaactgtgagctggatgtcacctgcaacatcaagaatggcagatgtgaacagttctgcaagaactcagcagacaacaaggtggtgtgcagctgcacagagggctacaggctggctgaaaaccagaagagctgtgaacctgctgtccctttcccctgtggcagagtgtctgtgtcccagacctccaagctgaccagggctgaaacagtgttccctgatgtggactatgtgaacagcactgaggctgagaccatcctggacaacatcacccagagcacccagagcttcaatgacttcaccagagtggtgggaggagaggatgccaagcctggccagttcccatggcaggtggtgctgaatggcaaggtggatgccttctgtgggggcagcattgtgaatgaaaaatggattgtgacagctgcccactgtgtggaaacaggtgtcaagatcacagtggtggctggtgaacacaacatagaagaaacagagcacacagagcagaagagaaatgtgatcaggatcatccctcaccacaactacaatgcagccatcaacaagtacaaccatgacattgccctgctggaacttgatgagcctctggtgctgaacagctatgttacccccatctgcattgcagacaaagagtacaccaacatcttcctgaagtttggctctggctacgtgtctggctggggcagagtgttccacaagggcaggtctgccctggtgctgcaatacctgagagtccccctggtggacagagccacctgcctgagaagcaccaagttcaccatctacaacaacatgttctgtgctggcttccatgagggaggcagagacagctgccaaggagactctggaggcccacacgtgacagaggtggagggcaccagcttcctgacaggcatcatcagctggggagaggaatgtgccatgaagggcaagtatggcatctacaccaaggtgtccagatatgtgaactggatcaaagaaaaaaccaaactgacctga

SEQ ID NO: 168 - DNA, 1296 bp, synthetic construct
  misc_feature 1..1296: note = Synthetic
  source 1..1296: mol_type = other DNA
ttcctggaccatgagaatgccaacaagatcctgaacagacccaagagatacaactctggcaagctggaggagtttgtgcaaggcaacctggagagagagtgcatggaagaaaagtgcagctttgaagaagccagggaagtgtttgaaaacacagagagaaccactgagttctggaagcagtatgtggatggagaccagtgtgagagcaacccttgcctgaatggaggcagctgcaaggatgacatcaacagctatgaatgctggtgcccttttggctttgaaggcaaaaactgtgagctggatgtcacctgcaacatcaagaatggcagatgtgaacagttctgcaagaactctgctgacaacaaggtggtgtgcagctgcacagagggctacaggctggcagaaaaccagaagtcctgtgaacctgctgtccctttcccctgtggcagagtgtctgtgtcccagaccagcaagctgaccagggctgagactgtgttccctgatgtggactatgtgaacagcacagaggctgaaaccatcctggacaacatcacccagagcacccagtccttcaatgacttcaccagggtggtgggtggagaggatgccaagcctggccagttcccatggcaggtggtgctgaatggcaaagtggatgccttctgtggaggcagcattgtgaatgagaaatggattgtgacagctgcccactgtgtggagacaggtgtcaaaatcacagtggtggctggagagcacaacatagaagaaacagagcacacagagcagaagagaaatgtgatcaggatcatccctcaccacaactacaatgcagccatcaacaagtacaaccatgacattgccctgctggaacttgatgagcctctggtgctgaacagctatgttacccccatctgcattgctgacaaggagtacaccaacatcttcctcaagtttggcagtggctatgtgtctggctggggcagagtgttccacaagggcaggtctgccctggtactgcaatacctgagagtccccctggtggacagggccacctgcctgagaagcaccaagttcaccatctacaacaacatgttctgtgctggcttccatgagggaggcagagacagctgccagggagactctggaggcccacatgtgacagaggtggagggcaccagcttcctgacaggcatcatcagctggggggaagagtgtgccatgaagggcaagtatggcatctacaccaaggttagcagatatgtgaactggatcaaagagaaaaccaagctgacctga

SEQ ID NO: 169 - DNA, 1296 bp, synthetic construct
  misc_feature 1..1296: note = Synthetic
  source 1..1296: mol_type = other DNA
ttcctggaccatgagaatgccaacaagatcctgaacagacccaagagatacaactctggcaagctggaggagtttgtgcaaggcaacctggagagagagtgcatggaagaaaagtgcagctttgaagaagcccgggaagtgtttgaaaacacagagagaaccactgagttctggaagcagtacgtggatggagaccagtgtgagagcaacccttgcctgaatggaggcagctgcaaggatgacatcaacagctacgaatgctggtgcccttttggctttgaaggcaaaaactgtgagctggatgtcacctgcaacatcaagaatggcagatgtgaacagttctgcaagaactctgctgacaacaaggtggtgtgcagctgcacagagggctacaggctggcagaaaaccagaagtcctgtgaacctgctgtccctttcccctgtggcagagtgtctgtgtcccagaccagcaagctgaccagggctgagactgtgttccctgatgtggactacgtgaacagcacagaggctgaaaccatcctggacaacatcacccagagcacccagtccttcaatgacttcacccgggtggtgggtggagaggatgccaagcctggccagttcccatggcaggtggtgctgaatggcaaagtggatgccttctgtggaggcagcattgtgaatgagaaatggattgtgacagccgcccactgtgtggagacaggtgtcaaaatcacagtggtggctggagagcacaacatagaagaaacagagcacacagagcagaagagaaatgtgatccggatcatccctcaccacaactacaatgcagccatcaacaagtacaaccacgacattgccctgctggaacttgatgagcctctggtgctgaacagctatgttacccccatctgcattgctgacaaggagtacaccaacatcttcctcaagtttggcagtggctatgtgtctggctggggcagagtgttccacaagggcaggtctgccctggtactgcaatacctgagagtccccctggtggacagggccacctgcctgagaagcaccaagttcaccatctacaacaacatgttctgtgccggcttccatgagggaggcagagacagctgccagggagactctggaggcccacacgtgacagaggtggagggcaccagcttcctgacaggcatcatcagctggggggaagagtgtgccatgaagggcaagtatggcatctacaccaaggttagcagatatgtgaactggatcaaagagaaaaccaagctgacctga

SEQ ID NO: 170 - DNA, 1296 bp, synthetic construct
  misc_feature 1..1296: note = Synthetic
  source 1..1296: mol_type = other DNA
ttcctggaccatgagaatgccaacaagatcctgaacagacccaagagatacaacagtggcaagctggaggaatttgtgcaaggcaacctggagagggagtgcatggaagagaagtgcagctttgaggaagccagggaagtgtttgagaacacagaaagaaccactgagttctggaagcagtatgttgatggagaccagtgtgagagcaacccttgcctgaatggaggctcctgcaaggatgacatcaacagctatgaatgctggtgcccttttggctttgaaggcaagaactgtgagctggatgtcacctgcaacatcaaaaatggcagatgtgaacagttctgcaagaactctgctgacaacaaggtggtgtgcagctgcacagagggctacaggctggcagaaaaccagaagtcctgtgaacctgctgtgcctttcccctgtggcagagtgtcagtgagccagaccagcaaactgaccagggctgagacagtgttccctgatgtggactatgtgaacagcacagaagctgaaaccatcctggacaacatcacccagagcacccagtccttcaatgacttcaccagggtggtgggtggagaggatgccaagcctggccagttcccatggcaggtggtgctcaatggcaaagtggatgccttctgtggaggcagcattgtgaatgaaaagtggattgtgacagctgcccactgtgtggagacaggtgtcaagatcacagtggtggctggagagcacaacattgaggaaacagagcacacagagcagaaaaggaatgtgatcagaatcatccctcaccacaactacaatgctgccatcaacaagtacaaccatgacattgccctgctggaacttgatgagcctctggtgctgaacagctatgttacccccatctgcattgctgacaaggagtacaccaacatcttcctgaagtttggctctggctatgtgtctggctggggcagagtgttccacaagggcaggtctgccctggtgctgcaatacctgagggtccccctggtggacagagccacctgcctgagaagcaccaagttcaccatctacaacaacatgttctgtgctggcttccatgagggaggcagagacagctgccagggagactctggaggcccacatgtgacagaggtggagggcaccagcttcctgacaggcatcatcagctggggggaagagtgtgccatgaagggcaagtatggcatctacaccaaggtgagcagatatgtcaactggatcaaagagaaaaccaagctgacctga

SEQ ID NO: 171 - DNA, 1296 bp, synthetic construct
  misc_feature 1..1296: note = Synthetic
  source 1..1296: mol_type = other DNA
ttcctggaccatgagaatgccaacaagatcctgaacagacccaagagatacaacagtggcaagctggaggaatttgtgcaaggcaacctggagagggagtgcatggaagagaagtgcagctttgaggaagcccgggaagtgttcgagaacacagaaagaaccactgagttctggaagcagtacgttgatggagaccagtgcgagagcaacccttgcctgaatggaggctcctgcaaggatgacatcaacagctacgaatgctggtgcccttttggctttgaaggcaagaactgtgagctggatgtcacctgcaacatcaaaaatggcagatgtgaacagttctgcaagaactctgctgacaacaaggtggtgtgcagctgcacagagggctacaggctggcagaaaaccagaagtcctgtgaacctgccgtgcctttcccctgtggcagagtgtcagtgagccagaccagcaaactgacccgggctgagacagtgttccctgatgtggactacgtgaacagcacagaagctgaaaccatcctggacaacatcacccagagcacccagtccttcaatgacttcacccgggtggtgggtggagaggatgccaagcctggccagttcccatggcaggtggtgctcaatggcaaagtggatgccttctgtggaggcagcattgtgaatgaaaagtggatcgtgacagccgcccactgtgtggagacaggtgtcaagatcacagtggtggctggagagcacaacatcgaggaaacagagcacacagagcagaaacggaatgtgatcagaatcatccctcaccacaactacaatgctgccatcaacaagtacaaccacgacattgccctgctggaacttgatgagcctctggtgctgaacagctatgttacccccatctgcattgccgacaaggagtacaccaacatcttcctgaagtttggctctggctacgtgtctggctggggcagagtgttccacaagggcaggtctgccctggtgctgcaatacctgcgggtccccctggtggacagagccacctgcctgagaagcaccaagttcaccatctacaacaacatgttctgtgccggcttccatgagggaggcagagacagctgccagggagactctggaggcccacacgtgacagaggtggagggcaccagcttcctgacaggcatcatcagctggggggaagagtgtgccatgaagggcaagtacggcatctacaccaaggtgagcagatatgtcaactggatcaaagagaaaaccaagctgacctga

SEQ ID NO: 172 - DNA, 3570 bp, synthetic construct
  misc_feature 1..3570: note = Synthetic
  misc_feature 1..145: note = ITR
  misc_feature 154..253: note = Splice acceptor
  misc_feature 256..1551: note = F9 wildtype
  misc_feature 1555..1782: note = bGH pA
  misc_feature 1889..2019: note = SV40 pA
  misc_feature 2020..3315: note = F9 codon optimized
  misc_feature 3318..3417: note = Splice acceptor
  misc_feature 3426..3570: note = ITR
  source 1..3570: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgttaggtgagcttagtcttttcttttatccaattcacgtagcgagagaccttcgtatagatgccatatttccccttcatcgcacattcctccccccaacttattatcccggtcaagaaacttgttccttcgacttcagtgacgtgtggtccacctgaatcaccttggcatgagtcgcgaccgccctcgtgaaacccagcacaaaacatgttattgtaaatcgtaaatttcgtggaccgaagacaggtcgctctatcgaccaacgggacgcgcaaatattgcagaacgagggctgatcgacctttgtggaagacccgcccccacccactcacatatccgctcccaaatttcaagaagatatttgtatattctttatcggctatacaaatcggggtaacataggagttaagtacgagtggctcgtccagctccaggagggctatatcatggttgtacttgtttatagcggcattataattgtgatggggtatgatcctgataacattccttttctgttcagtatgctcagtttcttcaatgttgtgttcgccagccacgaccgtaatcttaacccccgtctcgacacagtgtgcggccgttacaatccacttttcattgactatggagcccccacaaaacgcgtcgacttttccgttgagcaccacctgccatggaaattggccaggtttagcgtcctcgcccccgacaaccctagtaaagtcattaaatgactgtgtggattgtgttatattatcaagaatcgtttcggcttcagtagagttaacgtagtccacatcgggaaaaactgtctcggcccttgtcaactttgatgtctgggacacacttacccgaccgcacgggaagggcaccgccggttcacagctcttttgattctcagcgagccggtagccctcagtgcaactacacacaactttgttgtcggcggaatttttacagaattgctcgcatcgtccatttttaatgttgcaggtgacgtccaactcgcagttttttccttcaaaaccaaaagggcaccaacactcgtaggaatttatatcgtctttacaactccccccattcagacatggattagattcgcattggtccccatcgacatattgcttccagaactcagtggtccgttctgtattctcaaacacctcgcgcgcttcttcaaaactgcatttttcctccatacactctcgctccaagttcccttgcacgaattcttcaagctttcctgagttataccttttaggccggttaagtatcttattcgcgttttcgtggtccagaaaaactgtggaaacagggagagaaaaaccacacaacatatttaaagattgatgaagacaactaactgtaatatgctgctttttgttcttctcttcactgacctaagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 173 - DNA, 3570 bp, synthetic construct
  misc_feature 1..3570: note = Synthetic
  source 1..3570: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaagtcagcttggttttctccttaatccagttcacatatctggatacttttgtgtagatcccatactttcccttcatagcacactcctcaccccagctgataatcccagtcaggaagctggttccctccacctcagtcacatggggacccccagaatctccctggcagctgtctctaccaccttcatggaaccctgcacagaacatattgttgtagattgtaaacttggtgctcctcaagcaggttgctctatccaccagaggcaccctcaaatactgcaataccagggcagacctgcctttatggaacaccctgccccagccagacacatatccagacccaaatttcaagaagatgttggtgtactctttatcagcaatacagatgggtgtcacatagctgttcaagaccaggggttcatccagttccaggagggcaatgtcatggttgtacttgttgatagcagcattgtagttgtggtgggggataatcctgatcacattcctcttctgctcagtgtgctcagtttcctcaatattgtgctctccagccaccacagtaatcttgactccagtttccacacagtgggctgcagtcacaatccacttctcatttacaattgaacctccacaaaaggcatccacttttccattgagcactacctgccaggggaactgcccaggcttggcatcctcccctcctaccaccctggtgaagtcattgaagctctgtgtgctctgagtgatattgtcaaggattgtctcagcctcagtgctattcacataatctacatcaggaaagactgtttcagccctagtcagcttgctggtttggctcacagacactctcccacagggaaaaggcactgcaggttcacagctcttctggttttcagccagcctatatccctcagtgcagctacagaccactttgttatcagcagaattcttgcaaaattgctcacacctgccattcttgatattacaggtcacatcaagctcacagtttttcccctcaaatccaaagggacaccaacattcatagctgttgatatcatctttacagctccccccattcaagcagggattgctctcacattgatcaccatccacatactgcttccaaaattcagttgttctctctgtattttcaaacacttctctggcttcctcaaagctgcacttctcttccatacactctctctccagattcccctgtacaaactcttcaagcttgccagagttatacctctttggtctattcagaattttgttggcattttcatgatcaagaaaaactgtggaaacagggagagaaaaaccacacaacatatttaaagattgatgaagacaactaactgtaatatgctgctttttgttcttctcttcactgacctaagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 174 - DNA, 3570 bp, synthetic construct
  misc_feature 1..3570: note = Synthetic
  source 1..3570: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagcttggtcttctccttgatccagttcacatacctgctcaccttggtgtagatgccatacttgcccttcatggcacactcctccccccagctgatgatgcctgtcaggaagctggtgccctccacctctgtcacatgggggcccccagagtctccctggcagctgtctctgcctccctcatggaagccagcacagaacatgttgttgtagatggtgaacttggtgctcctcaggcaggtggccctgtccaccaggggcactctcaggtactgcagcaccagggcagacctgcccttgtggaacactctgccccagccagacacatagccagagccaaacttcaggaagatgttggtgtactccttgtcagcaatgcagatgggggtcacatagctgttcagcaccaggggctcatccagctccagcagggcaatgtcatggttgtacttgttgatggcagcattgtagttgtggtgggggatgatcctgatcacattcctcttctgctctgtgtgctctgtctcctcaatgttgtgctccccagccaccactgtgatcttcacccctgtctccacacagtgggcagctgtcacaatccacttctcattcacaatgctgcctccacagaaggcatccaccttgccattcagcaccacctgccaggggaactggccaggcttggcatcctctccccccaccaccctggtgaagtcattgaagctctgggtgctctgggtgatgttgtccaggatggtctcagcctctgtgctgttcacatagtccacatcagggaacactgtctcagccctggtcagcttgctggtctggctcacagacactctgccacaggggaagggcacagcaggctcacagctcttctggttctcagccagcctgtagccctctgtgcagctgcacaccaccttgttgtcagcagagttcttgcagaactgctcacatctgccattcttgatgttgcaggtcacatccagctcacagttcttgccctcaaagccaaaggggcaccagcactcatagctgttgatgtcatccttgcagctgcctccattcaggcaggggttgctctcacactggtctccatccacatactgcttccagaactctgtggttctctctgtgttctcaaacacctccctggcctcctcaaagctgcacttctcctccatgcactctctctccaggttgccctgcacaaactcctccagcttgccagagttgtacctcttgggcctgttcaggatcttgttggcattctcatggtccaggaaaactgtggaaacagggagagaaaaaccacacaacatatttaaagattgatgaagacaactaactgtaatatgctgctttttgttcttctcttcactgacctaagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 175 - DNA, 3570 bp, synthetic construct
  misc_feature 1..3570: note = Synthetic
  source 1..3570: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtgagcttggtcttttctttgatccagttgacatatctggacaccttggtgtagatgccatatttgcccttcatggcacactcttctccccagctgatgatgcctgtcagaaagctggtgccttccacctctgtcacatgagggccaccactatctccctggcaggagtctctgccaccttcatggaacccagcacagaacatgttgttgtagatggtgaacttggtgcttctcagacaggtggctctatccaccaggggcactctcaggtactgcagcaccagggcagaccttcccttgtggaacactctaccccagccagacacatagccagagccaaacttcagaaagatgttggtgtactctttgtcagcaatgcagataggggtcacataggagttcaggaccaggggctcatccagctcaagcagggcaatgtcatggttgtacttgttgatggcagcattgtagttgtggtgagggatgatcctgatgacattcctcttttgctctgtgtgctctgtttcctcaatgttgtgctccccagccaccactgtgatcttgacccctgtttccacacagtgggcagctgtcacaatccacttctcattcacaatggagccaccacagaaggcatccactttgccattcagcaccacttgccaagggaactgtccaggcttggcatcttcccctccaacaactcttgtgaagtcattgaaggactgggtgctctgggtgatgttgtccaggatggtttcagcctctgtggagttcacatagtccacatcagggaacactgtctcagctctggtcagcttgctggtctgggacacagacactctgccacatggaaagggcacagcaggttcacagcttttctggttctcagccagtctgtagccctctgtgcaggagcacacaaccttgttgtcagcagagttcttgcagaactgttcacatctgccattcttgatgttgcaggtcacatccagttcacagttcttgccctcaaagccaaaggggcaccagcactcatagctgttgatgtcatccttgcagctgccaccattcaggcaggggttgctttcacactggtccccatccacatactgcttccagaactctgtggttctctctgtgttctcaaacacttccctggcctcttcaaagctgcacttctcttccatgcattccctttccaggttgccctgcacaaactcttccagcttgccagagttgtacctcttgggcctgttcaggatcttgttggcattctcatggtccaggaaaactgtggaaacagggagagaaaaaccacacaacatatttaaagattgatgaagacaactaactgtaatatgctgctttttgttcttctcttcactgacctaagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 176 - DNA, 3570 bp, synthetic construct
  misc_feature 1..3570: note = Synthetic
  source 1..3570: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagtttggttttttctttgatccagttcacatatctggacaccttggtgtagatgccatacttgcccttcatggcacattcctctccccagctgatgatgcctgtcaggaagctggtgccctccacctctgtcacatgtgggcctccagagtctccttggcagctgtctctgcctccctcatggaagccagcacagaacatgttgttgtagatggtgaacttggtgcttctcaggcaggtggctctgtccaccagggggactctcaggtattgcagcaccagggcagacctgcccttgtggaacactctgccccagccagacacatagccagagccaaacttcaggaagatgttggtgtactctttgtctgcaatgcagatgggggtaacatagctgttcagcaccagaggctcatcaagttccagcagggcaatgtcatggttgtacttgttgatggctgcattgtagttgtggtgagggatgatcctgatcacatttctcttctgctctgtgtgctctgtttcttctatgttgtgttcaccagccaccactgtgatcttgacacctgtttccacacagtgggcagctgtcacaatccatttttcattcacaatgctgcccccacagaaggcatccaccttgccattcagcaccacctgccatgggaactggccaggcttggcatcctctcctcccaccactctggtgaagtcattgaagctctgggtgctctgggtgatgttgtccaggatggtctcagcctcagtgctgttcacatagtccacatcagggaacactgtttcagccctggtcagcttggaggtctgggacacagacactctgccacaggggaaagggacagcaggttcacagctcttctggttttcagccagcctgtagccctctgtgcagctgcacaccaccttgttgtctgctgagttcttgcagaactgttcacatctgccattcttgatgttgcaggtgacatccagctcacagttcttgccctcaaagccaaaagggcaccagcactcatagctgttgatgtcatctttgcagctgcctccattcaggcaagggttgctctcacactggtctccatcaacatactgcttccagaactcagtggttctctctgtgttttcaaacacttctctggcttcctcaaagctgcacttctcttccatgcactccctctccaggttgccctgcacaaactcctccagcttgccactgttgtatctcttgggtctgttgaggatcttgttggcattctcatggtccaggaaaactgtggaaacagggagagaaaaaccacacaacatatttaaagattgatgaagacaactaactgtaatatgctgctttttgttcttctcttcactgacctaagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 177 - DNA, 3570 bp, synthetic construct
  misc_feature 1..3570: note = Synthetic
  source 1..3570: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagtttttcttgatcatgaaaatgccaacaaaattctgaataggccaaagaggtataattcagggaaattggaagagtttgttcaagggaaccttgagagagaatgtatggaagaaaagtgtagttttgaagaagcaagagaagtttttgaaaacactgaaagaacaactgaattttggaagcagtatgttgatggagatcagtgcgagtccaatccatgtttaaatgggggcagttgcaaggatgacattaattcctatgaatgttggtgtccctttggatttgaaggaaagaactgtgaattagatgtaacatgtaacattaagaatggcagatgtgagcagttttgtaaaaatagtgctgataacaaggtggtttgctcctgtactgagggatatagacttgcagaaaaccagaagtcctgtgaaccagcagtgccatttccatgtggaagagtttctgtttcacaaacttctaagctcaccagagctgagactgtttttcctgatgtggactatgtaaattctactgaagctgaaaccattttggataacatcactcaaagcacccaatcatttaatgacttcactagggttgttggtggagaagatgccaaaccaggacaattcccttggcaggttgttttgaatggtaaagttgatgcattctgtggaggctctattgttaatgaaaaatggattgtaactgctgcccactgtgttgaaactggtgttaaaattacagttgtggcaggtgaacataatattgaggagacagaacatacagagcaaaagagaaatgtgattagaattattcctcaccacaactacaatgcagctattaataagtacaaccatgacattgcccttctggaactggatgaacccttagtgctaaacagctatgttacacctatttgcattgctgacaaggaatacaccaacatcttcctcaaatttggatctggctatgtaagtggctggggaagagtcttccacaaagggagatcagctttagttcttcagtaccttagagttccacttgttgacagagccacatgtcttaggtctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatccaggtatgtcaactggattaaggaaaaaacaaagctcacttaacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaagtcagcttggttttctccttaatccagttcacatatctggatacttttgtgtagatcccatactttcccttcatagcacactcctcaccccagctgataatcccagtcaggaagctggttccctccacctcagtcacatggggacccccagaatctccctggcagctgtctctaccaccttcatggaaccctgcacagaacatattgttgtagattgtaaacttggtgctcctcaagcaggttgctctatccaccagaggcaccctcaaatactgcaataccagggcagacctgcctttatggaacaccctgccccagccagacacatatccagacccaaatttcaagaagatgttggtgtactctttatcagcaatacagatgggtgtcacatagctgttcaagaccaggggttcatccagttccaggagggcaatgtcatggttgtacttgttgatagcagcattgtagttgtggtgggggataatcctgatcacattcctcttctgctcagtgtgctcagtttcctcaatattgtgctctccagccaccacagtaatcttgactccagtttccacacagtgggctgcagtcacaatccacttctcatttacaattgaacctccacaaaaggcatccacttttccattgagcactacctgccaggggaactgcccaggcttggcatcctcccctcctaccaccctggtgaagtcattgaagctctgtgtgctctgagtgatattgtcaaggattgtctcagcctcagtgctattcacataatctacatcaggaaagactgtttcagccctagtcagcttgctggtttggctcacagacactctcccacagggaaaaggcactgcaggttcacagctcttctggttttcagccagcctatatccctcagtgcagctacagaccactttgttatcagcagaattcttgcaaaattgctcacacctgccattcttgatattacaggtcacatcaagctcacagtttttcccctcaaatccaaagggacaccaacattcatagctgttgatatcatctttacagctccccccattcaagcagggattgctctcacattgatcaccatccacatactgcttccaaaattcagttgttctctctgtattttcaaacacttctctggcttcctcaaagctgcacttctcttccatacactctctctccagattcccctgtacaaactcttcaagcttgccagagttatacctctttggtctattcagaattttgttggcattttcatgatcaagaaaaactgtggaaacagggagagaaaaaccacacaacatatttaaagattgatgaagacaactaactgtaatatgctgctttttgttcttctcttcactgacctaagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 178 - DNA, 3570 bp, synthetic construct
  misc_feature 1..3570: note = Synthetic
  source 1..3570: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagtttttcttgatcatgaaaatgccaacaaaattctgaataggccaaagaggtataattcagggaaattggaagagtttgttcaagggaaccttgagagagaatgtatggaagaaaagtgtagttttgaagaagcaagagaagtttttgaaaacactgaaagaacaactgaattttggaagcagtatgttgatggagatcagtgcgagtccaatccatgtttaaatgggggcagttgcaaggatgacattaattcctatgaatgttggtgtccctttggatttgaaggaaagaactgtgaattagatgtaacatgtaacattaagaatggcagatgtgagcagttttgtaaaaatagtgctgataacaaggtggtttgctcctgtactgagggatatagacttgcagaaaaccagaagtcctgtgaaccagcagtgccatttccatgtggaagagtttctgtttcacaaacttctaagctcaccagagctgagactgtttttcctgatgtggactatgtaaattctactgaagctgaaaccattttggataacatcactcaaagcacccaatcatttaatgacttcactagggttgttggtggagaagatgccaaaccaggacaattcccttggcaggttgttttgaatggtaaagttgatgcattctgtggaggctctattgttaatgaaaaatggattgtaactgctgcccactgtgttgaaactggtgttaaaattacagttgtggcaggtgaacataatattgaggagacagaacatacagagcaaaagagaaatgtgattagaattattcctcaccacaactacaatgcagctattaataagtacaaccatgacattgcccttctggaactggatgaacccttagtgctaaacagctatgttacacctatttgcattgctgacaaggaatacaccaacatcttcctcaaatttggatctggctatgtaagtggctggggaagagtcttccacaaagggagatcagctttagttcttcagtaccttagagttccacttgttgacagagccacatgtcttaggtctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatccaggtatgtcaactggattaaggaaaaaacaaagctcacttaacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagcttggtcttctccttgatccagttcacatacctgctcaccttggtgtagatgccatacttgcccttcatggcacactcctccccccagctgatgatgcctgtcaggaagctggtgccctccacctctgtcacatgggggcccccagagtctccctggcagctgtctctgcctccctcatggaagccagcacagaacatgttgttgtagatggtgaacttggtgctcctcaggcaggtggccctgtccaccaggggcactctcaggtactgcagcaccagggcagacctgcccttgtggaacactctgccccagccagacacatagccagagccaaacttcaggaagatgttggtgtactccttgtcagcaatgcagatgggggtcacatagctgttcagcaccaggggctcatccagctccagcagggcaatgtcatggttgtacttgttgatggcagcattgtagttgtggtgggggatgatcctgatcacattcctcttctgctctgtgtgctctgtctcctcaatgttgtgctccccagccaccactgtgatcttcacccctgtctccacacagtgggcagctgtcacaatccacttctcattcacaatgctgcctccacagaaggcatccaccttgccattcagcaccacctgccaggggaactggccaggcttggcatcctctccccccaccaccctggtgaagtcattgaagctctgggtgctctgggtgatgttgtccaggatggtctcagcctctgtgctgttcacatagtccacatcagggaacactgtctcagccctggtcagcttgctggtctggctcacagacactctgccacaggggaagggcacagcaggctcacagctcttctggttctcagccagcctgtagccctctgtgcagctgcacaccaccttgttgtcagcagagttcttgcagaactgctcacatctgccattcttgatgttgcaggtcacatccagctcacagttcttgccctcaaagccaaaggggcaccagcactcatagctgttgatgtcatccttgcagctgcctccattcaggcaggggttgctctcacactggtctccatccacatactgcttccagaactctgtggttctctctgtgttctcaaacacctccctggcctcctcaaagctgcacttctcctccatgcactctctctccaggttgccctgcacaaactcctccagcttgccagagttgtacctcttgggcctgttcaggatcttgttggcattctcatggtccaggaaaactgtggaaacagggagagaaaaaccacacaacatatttaaagattgatgaagacaactaactgtaatatgctgctttttgttcttctcttcactgacctaagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 179 - DNA, 3570 bp, synthetic construct
  misc_feature 1..3570: note = Synthetic
  misc_feature 1..145: note = ITR
  misc_feature 154..253: note = Splice acceptor
  misc_feature 256..1551: note = F9 CDS #1 Native CpG Removed No Splice F9
  misc_feature 315: note = Cryptic splice donor site mutated
  misc_feature 456: note = Cryptic splice donor site mutated
  misc_feature 873: note = Cryptic splice donor site mutated
  misc_feature 1555..1556: note = CpG removed
  misc_feature 1558..1781: note = bGH pA with CpG sites removed
  misc_feature 1792..1793: note = CpG removed
  misc_feature 1889..2019: note = SV40 pA
  misc_feature 2020..3315: note = F9 CDS #2 (GA F9)
  misc_feature 3318..3417: note = Splice acceptor
  misc_feature 3426..3570: note = ITR
  source 1..3570: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagtttttcttgatcatgaaaatgccaacaaaattctgaataggccaaagaggtataattcagggaaattggaagagtttgttcaagggaaccttgagagagaatgtatggaagaaaagtgtagttttgaagaagcaagagaagtttttgaaaacactgaaagaacaactgaattttggaagcagtatgttgatggagatcagtgcgagtccaatccatgtttaaatgggggcagttgcaaggatgacattaattcctatgaatgttggtgtccctttggatttgaaggaaagaactgtgaattagatgtaacatgtaacattaagaatggcagatgtgagcagttttgtaaaaatagtgctgataacaaggtggtttgctcctgtactgagggatatagacttgcagaaaaccagaagtcctgtgaaccagcagtgccatttccatgtggaagagtttctgtttcacaaacttctaagctcaccagagctgagactgtttttcctgatgtggactatgtaaattctactgaagctgaaaccattttggataacatcactcaaagcacccaatcatttaatgacttcactagggttgttggtggagaagatgccaaaccaggacaattcccttggcaggttgttttgaatggtaaagttgatgcattctgtggaggctctattgttaatgaaaaatggattgtaactgctgcccactgtgttgaaactggtgttaaaattacagttgtggcaggtgaacataatattgaggagacagaacatacagagcaaaagagaaatgtgattagaattattcctcaccacaactacaatgcagctattaataagtacaaccatgacattgcccttctggaactggatgaacccttagtgctaaacagctatgttacacctatttgcattgctgacaaggaatacaccaacatcttcctcaaatttggatctggctatgtaagtggctggggaagagtcttccacaaagggagatcagctttagttcttcagtaccttagagttccacttgttgacagagccacatgtcttaggtctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatccaggtatgtcaactggattaaggaaaaaacaaagctcacttaacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtgagcttggtcttttctttgatccagttgacatatctggacaccttggtgtagatgccatatttgcccttcatggcacactcttctccccagctgatgatgcctgtcagaaagctggtgccttccacctctgtcacatgagggccaccactatctccctggcaggagtctctgccaccttcatggaacccagcacagaacatgttgttgtagatggtgaacttggtgcttctcagacaggtggctctatccaccaggggcactctcaggtactgcagcaccagggcagaccttcccttgtggaacactctaccccagccagacacatagccagagccaaacttcagaaagatgttggtgtactctttgtcagcaatgcagataggggtcacataggagttcaggaccaggggctcatccagctcaagcagggcaatgtcatggttgtacttgttgatggcagcattgtagttgtggtgagggatgatcctgatgacattcctcttttgctctgtgtgctctgtttcctcaatgttgtgctccccagccaccactgtgatcttgacccctgtttccacacagtgggcagctgtcacaatccacttctcattcacaatggagccaccacagaaggcatccactttgccattcagcaccacttgccaagggaactgtccaggcttggcatcttcccctccaacaactcttgtgaagtcattgaaggactgggtgctctgggtgatgttgtccaggatggtttcagcctctgtggagttcacatagtccacatcagggaacactgtctcagctctggtcagcttgctggtctgggacacagacactctgccacatggaaagggcacagcaggttcacagcttttctggttctcagccagtctgtagccctctgtgcaggagcacacaaccttgttgtcagcagagttcttgcagaactgttcacatctgccattcttgatgttgcaggtcacatccagttcacagttcttgccctcaaagccaaaggggcaccagcactcatagctgttgatgtcatccttgcagctgccaccattcaggcaggggttgctttcacactggtccccatccacatactgcttccagaactctgtggttctctctgtgttctcaaacacttccctggcctcttcaaagctgcacttctcttccatgcattccctttccaggttgccctgcacaaactcttccagcttgccagagttgtacctcttgggcctgttcaggatcttgttggcattctcatggtccaggaaaactgtggaaacagggagagaaaaaccacacaacatatttaaagattgatgaagacaactaactgtaatatgctgctttttgttcttctcttcactgacctaagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 180 - DNA, 3570 bp, synthetic construct
  misc_feature 1..3570: note = Synthetic
  misc_feature 1..145: note = ITR
  misc_feature 154..253: note = Splice acceptor
  misc_feature 256..1551: note = F9 CDS #1 Native CpG Removed No Splice F9
  misc_feature 315: note = Cryptic splice donor site mutated
  misc_feature 456: note = Cryptic splice donor site mutated
  misc_feature 873: note = Cryptic splice donor site mutated
  misc_feature 1555..1556: note = CpG removed
  misc_feature 1558..1781: note = bGH pA with CpG sites removed
  misc_feature 1792..1793: note = CpG removed
  misc_feature 1889..2019: note = SV40 pA
  misc_feature 2020..3315: note = F9 CDS #2
  misc_feature 3318..3417: note = Splice acceptor
  misc_feature 3426..3570: note = ITR
  source 1..3570: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagtttttcttgatcatgaaaatgccaacaaaattctgaataggccaaagaggtataattcagggaaattggaagagtttgttcaagggaaccttgagagagaatgtatggaagaaaagtgtagttttgaagaagcaagagaagtttttgaaaacactgaaagaacaactgaattttggaagcagtatgttgatggagatcagtgcgagtccaatccatgtttaaatgggggcagttgcaaggatgacattaattcctatgaatgttggtgtccctttggatttgaaggaaagaactgtgaattagatgtaacatgtaacattaagaatggcagatgtgagcagttttgtaaaaatagtgctgataacaaggtggtttgctcctgtactgagggatatagacttgcagaaaaccagaagtcctgtgaaccagcagtgccatttccatgtggaagagtttctgtttcacaaacttctaagctcaccagagctgagactgtttttcctgatgtggactatgtaaattctactgaagctgaaaccattttggataacatcactcaaagcacccaatcatttaatgacttcactagggttgttggtggagaagatgccaaaccaggacaattcccttggcaggttgttttgaatggtaaagttgatgcattctgtggaggctctattgttaatgaaaaatggattgtaactgctgcccactgtgttgaaactggtgttaaaattacagttgtggcaggtgaacataatattgaggagacagaacatacagagcaaaagagaaatgtgattagaattattcctcaccacaactacaatgcagctattaataagtacaaccatgacattgcccttctggaactggatgaacccttagtgctaaacagctatgttacacctatttgcattgctgacaaggaatacaccaacatcttcctcaaatttggatctggctatgtaagtggctggggaagagtcttccacaaagggagatcagctttagttcttcagtaccttagagttccacttgttgacagagccacatgtcttaggtctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatccaggtatgtcaactggattaaggaaaaaacaaagctcacttaacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagtttggttttttctttgatccagttcacatatctggacaccttggtgtagatgccatacttgcccttcatggcacattcctctccccagctgatgatgcctgtcaggaagctggtgccctccacctctgtcacatgtgggcctccagagtctccttggcagctgtctctgcctccctcatggaagccagcacagaacatgttgttgtagatggtgaacttggtgcttctcaggcaggtggctctgtccaccagggggactctcaggtattgcagcaccagggcagacctgcccttgtggaacactctgccccagccagacacatagccagagccaaacttcaggaagatgttggtgtactctttgtctgcaatgcagatgggggtaacatagctgttcagcaccagaggctcatcaagttccagcagggcaatgtcatggttgtacttgttgatggctgcattgtagttgtggtgagggatgatcctgatcacatttctcttctgctctgtgtgctctgtttcttctatgttgtgttcaccagccaccactgtgatcttgacacctgtttccacacagtgggcagctgtcacaatccatttttcattcacaatgctgcccccacagaaggcatccaccttgccattcagcaccacctgccatgggaactggccaggcttggcatcctctcctcccaccactctggtgaagtcattgaagctctgggtgctctgggtgatgttgtccaggatggtctcagcctcagtgctgttcacatagtccacatcagggaacactgtttcagccctggtcagcttggaggtctgggacacagacactctgccacaggggaaagggacagcaggttcacagctcttctggttttcagccagcctgtagccctctgtgcagctgcacaccaccttgttgtctgctgagttcttgcagaactgttcacatctgccattcttgatgttgcaggtgacatccagctcacagttcttgccctcaaagccaaaagggcaccagcactcatagctgttgatgtcatctttgcagctgcctccattcaggcaagggttgctctcacactggtctccatcaacatactgcttccagaactcagtggttctctctgtgttttcaaacacttctctggcttcctcaaagctgcacttctcttccatgcactccctctccaggttgccctgcacaaactcctccagcttgccactgttgtatctcttgggtctgttgaggatcttgttggcattctcatggtccaggaaaactgtggaaacagggagagaaaaaccacacaacatatttaaagattgatgaagacaactaactgtaatatgctgctttttgttcttctcttcactgacctaagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 181 - DNA, 3570 bp, synthetic construct
  misc_feature 1..3570: note = Synthetic
  source 1..3570: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagtttttcttgatcatgaaaatgccaacaaaattctgaataggccaaagaggtataattcagggaaattggaagagtttgttcaagggaaccttgagagagaatgtatggaagaaaagtgtagttttgaagaagcaagagaagtttttgaaaacactgaaagaacaactgaattttggaagcagtatgttgatggagatcagtgcgagtccaatccatgtttaaatgggggcagttgcaaggatgacattaattcctatgaatgttggtgtccctttggatttgaaggaaagaactgtgaattagatgtaacatgtaacattaagaatggcagatgtgagcagttttgtaaaaatagtgctgataacaaggtggtttgctcctgtactgagggatatagacttgcagaaaaccagaagtcctgtgaaccagcagtgccatttccatgtggaagagtttctgtttcacaaacttctaagctcaccagagctgagactgtttttcctgatgtggactatgtaaattctactgaagctgaaaccattttggataacatcactcaaagcacccaatcatttaatgacttcactagggttgttggtggagaagatgccaaaccaggacaattcccttggcaggttgttttgaatggtaaagttgatgcattctgtggaggctctattgttaatgaaaaatggattgtaactgctgcccactgtgttgaaactggtgttaaaattacagttgtggcaggtgaacataatattgaggagacagaacatacagagcaaaagagaaatgtgattagaattattcctcaccacaactacaatgcagctattaataagtacaaccatgacattgcccttctggaactggatgaacccttagtgctaaacagctatgttacacctatttgcattgctgacaaggaatacaccaacatcttcctcaaatttggatctggctatgtaagtggctggggaagagtcttccacaaagggagatcagctttagttcttcagtaccttagagttccacttgttgacagagccacatgtcttaggtctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatccaggtatgtcaactggattaaggaaaaaacaaagctcacttaacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgttaggtgagcttagtcttttcttttatccaattcacgtagcgagagaccttcgtatagatgccatatttccccttcatcgcacattcctccccccaacttattatcccggtcaagaaacttgttccttcgacttcagtgacgtgtggtccacctgaatcaccttggcatgagtcgcgaccgccctcgtgaaacccagcacaaaacatgttattgtaaatcgtaaatttcgtggaccgaagacaggtcgctctatcgaccaacgggacgcgcaaatattgcagaacgagggctgaccgacctttgtggaagacccgcccccacccactcacatatccgctcccaaatttcaagaagatatttgtatattctttatcggctatacaaatcggggtaacataggagttaagtacgagtggctcgtccagctccaggagggctatatcatggttgtacttgtttatagcggcattataattgtgatggggtatgatcctgataacattccttttctgttcagtatgctcagtttcttcaatgttgtgttcgccagccacgaccgtaatcttaacccccgtctcgacacagtgtgcggccgttacaatccacttttcattgactatggagcccccacaaaacgcgtcgacttttccgttgagcaccacctgccatggaaattggccaggtttagcgtcctcgcccccgacaaccctagtaaagtcattaaatgactgtgtggattgtgttatattatcaagaatcgtttcggcttcagtagagttaacgtagtccacatcgggaaaaactgtctcggcccttgtcaactttgatgtctgggacacacttacccgaccgcacgggaagggcaccgccggttcacagctcttttgattctcagcgagccggtagccctcagtgcaactacacacaactttgttgtcggcggaatttttacagaattgctcgcatcgtccatttttaatgttgcaggtgacgtccaactcgcagttttttccttcaaaaccaaaagggcaccaacactcgtaggaatttatatcgtctttacaactccccccattcagacatggattagattcgcattggtccccatcgacatattgcttccagaactcagtggtccgttctgtattctcaaacacctcgcgcgcttcttcaaaactgcatttttcctccatacactctcgctccaagttcccttgcacgaattcttcaagctttcctgagttataccttttaggccggttaagtatcttattcgcgttttcgtggtccagaaaaactgtggaaacagggagagaaaaaccacacaacatatttaaagattgatgaagacaactaactgtaatatgctgctttttgttcttctcttcactgacctaagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 182 - DNA, 3570 bp, synthetic construct
  misc_feature 1..3570: note = Synthetic
  source 1..3570: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagtttttcttgatcatgaaaatgccaacaaaattctgaataggccaaagaggtataattcaggtaaattggaagagtttgttcaagggaaccttgagagagaatgtatggaagaaaagtgtagttttgaagaagcaagagaagtttttgaaaacactgaaagaacaactgaattttggaagcagtatgttgatggagatcagtgtgagtccaatccatgtttaaatgggggcagttgcaaggatgacattaattcctatgaatgttggtgtccctttggatttgaaggaaagaactgtgaattagatgtaacatgtaacattaagaatggcagatgtgagcagttttgtaaaaatagtgctgataacaaggtggtttgctcctgtactgagggatatagacttgcagaaaaccagaagtcctgtgaaccagcagtgccatttccatgtggaagagtttctgtttcacaaacttctaagctcaccagagctgagactgtttttcctgatgtggactatgtaaattctactgaagctgaaaccattttggataacatcactcaaagcacccaatcatttaatgacttcactagggttgttggtggagaagatgccaaaccaggtcaattcccttggcaggttgttttgaatggtaaagttgatgcattctgtggaggctctattgttaatgaaaaatggattgtaactgctgcccactgtgttgaaactggtgttaaaattacagttgtggcaggtgaacataatattgaggagacagaacatacagagcaaaagagaaatgtgattagaattattcctcaccacaactacaatgcagctattaataagtacaaccatgacattgcccttctggaactggatgaacccttagtgctaaacagctatgttacacctatttgcattgctgacaaggaatacaccaacatcttcctcaaatttggatctggctatgtaagtggctggggaagagtcttccacaaagggagatcagctttagttcttcagtaccttagagttccacttgttgacagagccacatgtcttaggtctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatccaggtatgtcaactggattaaggaaaaaacaaagctcacttaacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgttaggtgagcttagtcttttcttttatccaattcacgtagcgagagaccttcgtatagatgccatatttccccttcatcgcacattcctccccccaacttattatcccggtcaagaaacttgttccttcgacttcagtgacgtgtggtccacctgaatcaccttggcatgagtcgcgaccgccctcgtgaaacccagcacaaaacatgttattgtaaatcgtaaatttcgtggaccgaagacaggtcgctctatcgaccaacgggacgcgcaaatattgcagaacgagggctgaccgacctttgtggaagacccgcccccacccactcacatatccgctcccaaatttcaagaagatatttgtatattctttatcggctatacaaatcggggtaacataggagttaagtacgagtggctcgtccagctccaggagggctatatcatggttgtacttgtttatagcggcattataattgtgatggggtatgatcctgataacattccttttctgttcagtatgctcagtttcttcaatgttgtgttcgccagccacgaccgtaatcttaacccccgtctcgacacagtgtgcggccgttacaatccacttttcattgactatggagcccccacaaaacgcgtcgacttttccgttgagcaccacctgccatggaaattggccaggtttagcgtcctcgcccccgacaaccctagtaaagtcattaaatgactgtgtggattgtgttatattatcaagaatcgtttcggcttcagtagagttaacgtagtccacatcgggaaaaactgtctcggcccttgtcaactttgatgtctgggacacacttacccgaccgcacgggaagggcaccgccggttcacagctcttttgattctcagcgagccggtagccctcagtgcaactacacacaactttgttgtcggcggaatttttacagaattgctcgcatcgtccatttttaatgttgcaggtgacgtccaactcgcagttttttccttcaaaaccaaaagggcaccaacactcgtaggaatttatatcgtctttacaactccccccattcagacatggattagattcgcattggtccccatcgacatattgcttccagaactcagtggtccgttctgtattctcaaacacctcgcgcgcttcttcaaaactgcatttttcctccatacactctcgctccaagttcccttgcacgaattcttcaagctttcctgagttataccttttaggccggttaagtatcttattcgcgttttcgtggtccagaaaaactgtggaaacagggagagaaaaaccacacaacatatttaaagattgatgaagacaactaactgtaatatgctgctttttgttcttctcttcactgacctaagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 183 - DNA, 3570 bp, synthetic construct
  misc_feature 1..3570: note = Synthetic
  source 1..3570: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgttaggtgagcttagtcttttcttttatccaattcacgtagcgagagaccttcgtatagatgccatatttccccttcatcgcacattcctccccccaacttattatcccggtcaagaaacttgttccttcgacttcagtgacgtgtggtccacctgaatcaccttggcatgagtcgcgaccgccctcgtgaaacccagcacaaaacatgttattgtaaatcgtaaatttcgtggaccgaagacaggtcgctctatcgaccaacgggacgcgcaaatattgcagaacgagggctgaccgacctttgtggaagacccgcccccacccactcacatatccgctcccaaatttcaagaagatatttgtatattctttatcggctatacaaatcggggtaacataggagttaagtacgagtggctcgtccagctccaggagggctatatcatggttgtacttgtttatagcggcattataattgtgatggggtatgatcctgataacattccttttctgttcagtatgctcagtttcttcaatgttgtgttcgccagccacgaccgtaatcttaacccccgtctcgacacagtgtgcggccgttacaatccacttttcattgactatggagcccccacaaaacgcgtcgacttttccgttgagcaccacctgccatggaaattggccaggtttagcgtcctcgcccccgacaaccctagtaaagtcattaaatgactgtgtggattgtgttatattatcaagaatcgtttcggcttcagtagagttaacgtagtccacatcgggaaaaactgtctcggcccttgtcaactttgatgtctgggacacacttacccgaccgcacgggaagggcaccgccggttcacagctcttttgattctcagcgagccggtagccctcagtgcaactacacacaactttgttgtcggcggaatttttacagaattgctcgcatcgtccatttttaatgttgcaggtgacgtccaactcgcagttttttccttcaaaaccaaaagggcaccaacactcgtaggaatttatatcgtctttacaactccccccattcagacatggattagattcgcattggtccccatcgacatattgcttccagaactcagtggtccgttctgtattctcaaacacctcgcgcgcttcttcaaaactgcatttttcctccatacactctcgctccaagttcccttgcacgaattcttcaagctttcctgagttataccttttaggccggttaagtatcttattcgcgttttcgtggtccagaaaaactgtggaaacagggagagaaaaaccacacaacatatttaaagattgatgaagacaactaactgtaatatgctgctttttgttcttctcttcactgacctaagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 184 - DNA, 3570 bp, synthetic construct
  misc_feature 1..3570: note = Synthetic
  source 1..3570: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcagggaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgcgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggacaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgttaggtgagcttagtcttttcttttatccaattcacgtagcgagagaccttcgtatagatgccatatttccccttcatcgcacattcctccccccaacttattatcccggtcaagaaacttgttccttcgacttcagtgacgtgtggtccacctgaatcaccttggcatgagtcgcgaccgccctcgtgaaacccagcacaaaacatgttattgtaaatcgtaaatttcgtggaccgaagacaggtcgctctatcgaccaacgggacgcgcaaatattgcagaacgagggctgatcgacctttgtggaagacccgcccccacccactcacatatccgctcccaaatttcaagaagatatttgtatattctttatcggctatacaaatcggggtaacataggagttaagtacgagtggctcgtccagctccaggagggctatatcatggttgtacttgtttatagcggcattataattgtgatggggtatgatcctgataacattccttttctgttcagtatgctcagtttcttcaatgttgtgttcgccagccacgaccgtaatcttaacccccgtctcgacacagtgtgcggccgttacaatccacttttcattgactatggagcccccacaaaacgcgtcgacttttccgttgagcaccacctgccatggaaattggccaggtttagcgtcctcgcccccgacaaccctagtaaagtcattaaatgactgtgtggattgtgttatattatcaagaatcgtttcggcttcagtagagttaacgtagtccacatcgggaaaaactgtctcggcccttgtcaactttgatgtctgggacacacttacccgaccgcacgggaagggcaccgccggttcacagctcttttgattctcagcgagccggtagccctcagtgcaactacacacaactttgttgtcggcggaatttttacagaattgctcgcatcgtccatttttaatgttgcaggtgacgtccaactcgcagttttttccttcaaaaccaaaagggcaccaacactcgtaggaatttatatcgtctttacaactccccccattcagacatggattagattcgcattggtccccatcgacatattgcttccagaactcagtggtccgttctgtattctcaaacacctcgcgcgcttcttcaaaactgcatttttcctccatacactctcgctccaagttcccttgcacgaattcttcaagctttcctgagttataccttttaggccggttaagtatcttattcgcgttttcgtggtccagaaaaactgtggaaacagggagagaaaaaccacacaacatatttaaagattgatgaagacaactaactgtaatatgctgctttttgttcttctcttcactgacctaagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 185 - DNA, 3570 bp, synthetic construct
  misc_feature 1..3570: note = Synthetic
  source 1..3570: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagtttggttttttctttgatccagttcacatatctggacaccttggtgtagatgccatacttgcccttcatggcacattcctctccccagctgatgatgcctgtcaggaagctggtgccctccacctctgtcacgtgtgggcctccagagtctccttggcagctgtctctgcctccctcatggaagccagcacagaacatgttgttgtagatggtgaacttggtgcttctcaggcaggtggctctgtccaccagggggactctcaggtattgcagcaccagggcagacctgcccttgtggaacactctgccccagccagacacgtagccagagccaaacttcaggaagatgttggtgtactctttgtctgcaatgcagatgggggtaacatagctgttcagcaccagaggctcatcaagttccagcagggcaatgtcatggttgtacttgttgatggctgcattgtagttgtggtgagggatgatcctgatcacatttctcttctgctctgtgtgctctgtttcttctatgttgtgttcaccagccaccactgtgatcttgacacctgtttccacacagtgggcagctgtcacaatccatttttcattcacaatgctgcccccacagaaggcatccaccttgccattcagcaccacctgccatgggaactggccaggcttggcatcctctcctcccaccactctggtgaagtcattgaagctctgggtgctctgggtgatgttgtccaggatggtctcagcctcagtgctgttcacatagtccacatcagggaacactgtttcagccctggtcagcttggaggtctgggacacagacactctgccacaggggaaagggacagcaggttcacagctcttctggttttcagccagcctgtagccctctgtgcagctgcacaccaccttgttgtctgctgagttcttgcagaactgttcacatctgccattcttgatgttgcaggtgacatccagctcacagttcttgccctcaaagccaaaagggcaccagcactcatagctgttgatgtcatctttgcagctgcctccattcaggcaagggttgctctcacactggtctccatcaacgtactgcttccagaactcagtggttctctctgtgttttcaaacacttctctggcttcctcaaagctgcacttctcttccatgcactccctctccaggttgccctgcacaaactcctccagcttgccactgttgtatctcttgggtctgttgaggatcttgttggcattctcatggtccaggaaaactgtggaaacagggagagaaaaaccacacaacatatttaaagattgatgaagacaactaactgtaatatgctgctttttgttcttctcttcactgacctaagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 186 - DNA, 3570 bp, synthetic construct
  misc_feature 1..3570: note = Synthetic
  source 1..3570: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagcttggttttctctttgatccagttcacatatctgctaaccttggtgtagatgccatacttgcccttcatggcacactcttccccccagctgatgatgcctgtcaggaagctggtgccctccacctctgtcacatgtgggcctccagagtctccctggcagctgtctctgcctccctcatggaagccagcacagaacatgttgttgtagatggtgaacttggtgcttctcaggcaggtggccctgtccaccagggggactctcaggtattgcagtaccagggcagacctgcccttgtggaacactctgccccagccagacacatagccactgccaaacttgaggaagatgttggtgtactccttgtcagcaatgcagatgggggtaacatagctgttcagcaccagaggctcatcaagttccagcagggcaatgtcatggttgtacttgttgatggctgcattgtagttgtggtgagggatgatcctgatcacatttctcttctgctctgtgtgctctgtttcttctatgttgtgctctccagccaccactgtgattttgacacctgtctccacacagtgggcagctgtcacaatccatttctcattcacaatgctgcctccacagaaggcatccactttgccattcagcaccacctgccatgggaactggccaggcttggcatcctctccacccaccaccctggtgaagtcattgaaggactgggtgctctgggtgatgttgtccaggatggtttcagcctctgtgctgttcacatagtccacatcagggaacacagtctcagccctggtcagcttgctggtctgggacacagacactctgccacaggggaaagggacagcaggttcacaggacttctggttttctgccagcctgtagccctctgtgcagctgcacaccaccttgttgtcagcagagttcttgcagaactgttcacatctgccattcttgatgttgcaggtgacatccagctcacagtttttgccttcaaagccaaaagggcaccagcattcatagctgttgatgtcatccttgcagctgcctccattcaggcaagggttgctctcacactggtctccatccacatactgcttccagaactcagtggttctctctgtgttttcaaacacttccctggcttcttcaaagctgcacttttcttccatgcactctctctccaggttgccttgcacaaactcctccagcttgccagagttgtatctcttgggtctgttcaggatcttgttggcattctcatggtccaggaaaactgtggaaacagggagagaaaaaccacacaacatatttaaagattgatgaagacaactaactgtaatatgctgctttttgttcttctcttcactgacctaagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 187 - DNA, 3570 bp, synthetic construct
  misc_feature 1..3570: note = Synthetic
  source 1..3570: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagcttggttttctctttgatccagttcacatatctgctaaccttggtgtagatgccatacttgcccttcatggcacactcttccccccagctgatgatgcctgtcaggaagctggtgccctccacctctgtcacgtgtgggcctccagagtctccctggcagctgtctctgcctccctcatggaagccggcacagaacatgttgttgtagatggtgaacttggtgcttctcaggcaggtggccctgtccaccagggggactctcaggtattgcagtaccagggcagacctgcccttgtggaacactctgccccagccagacacatagccactgccaaacttgaggaagatgttggtgtactccttgtcagcaatgcagatgggggtaacatagctgttcagcaccagaggctcatcaagttccagcagggcaatgtcgtggttgtacttgttgatggctgcattgtagttgtggtgagggatgatccggatcacatttctcttctgctctgtgtgctctgtttcttctatgttgtgctctccagccaccactgtgattttgacacctgtctccacacagtgggcggctgtcacaatccatttctcattcacaatgctgcctccacagaaggcatccactttgccattcagcaccacctgccatgggaactggccaggcttggcatcctctccacccaccacccgggtgaagtcattgaaggactgggtgctctgggtgatgttgtccaggatggtttcagcctctgtgctgttcacgtagtccacatcagggaacacagtctcagccctggtcagcttgctggtctgggacacagacactctgccacaggggaaagggacagcaggttcacaggacttctggttttctgccagcctgtagccctctgtgcagctgcacaccaccttgttgtcagcagagttcttgcagaactgttcacatctgccattcttgatgttgcaggtgacatccagctcacagtttttgccttcaaagccaaaagggcaccagcattcgtagctgttgatgtcatccttgcagctgcctccattcaggcaagggttgctctcacactggtctccatccacgtactgcttccagaactcagtggttctctctgtgttttcaaacacttcccgggcttcttcaaagctgcacttttcttccatgcactctctctccaggttgccttgcacaaactcctccagcttgccagagttgtatctcttgggtctgttcaggatcttgttggcattctcatggtccaggaaaactgtggaaacagggagagaaaaaccacacaacatatttaaagattgatgaagacaactaactgtaatatgctgctttttgttcttctcttcactgacctaagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 188 - DNA, 3570 bp, synthetic construct
  misc_feature 1..3570: note = Synthetic
  source 1..3570: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagcttggttttctctttgatccagttgacatatctgctcaccttggtgtagatgccatacttgcccttcatggcacactcttccccccagctgatgatgcctgtcaggaagctggtgccctccacctctgtcacatgtgggcctccagagtctccctggcagctgtctctgcctccctcatggaagccagcacagaacatgttgttgtagatggtgaacttggtgcttctcaggcaggtggctctgtccaccagggggaccctcaggtattgcagcaccagggcagacctgcccttgtggaacactctgccccagccagacacatagccagagccaaacttcaggaagatgttggtgtactccttgtcagcaatgcagatgggggtaacatagctgttcagcaccagaggctcatcaagttccagcagggcaatgtcatggttgtacttgttgatggcagcattgtagttgtggtgagggatgattctgatcacattccttttctgctctgtgtgctctgtttcctcaatgttgtgctctccagccaccactgtgatcttgacacctgtctccacacagtgggcagctgtcacaatccacttttcattcacaatgctgcctccacagaaggcatccactttgccattgagcaccacctgccatgggaactggccaggcttggcatcctctccacccaccaccctggtgaagtcattgaaggactgggtgctctgggtgatgttgtccaggatggtttcagcttctgtgctgttcacatagtccacatcagggaacactgtctcagccctggtcagtttgctggtctggctcactgacactctgccacaggggaaaggcacagcaggttcacaggacttctggttttctgccagcctgtagccctctgtgcagctgcacaccaccttgttgtcagcagagttcttgcagaactgttcacatctgccatttttgatgttgcaggtgacatccagctcacagttcttgccttcaaagccaaaagggcaccagcattcatagctgttgatgtcatccttgcaggagcctccattcaggcaagggttgctctcacactggtctccatcaacatactgcttccagaactcagtggttctttctgtgttctcaaacacttccctggcttcctcaaagctgcacttctcttccatgcactccctctccaggttgccttgcacaaattcctccagcttgccactgttgtatctcttgggtctgttcaggatcttgttggcattctcatggtccaggaaaactgtggaaacagggagagaaaaaccacacaacatatttaaagattgatgaagacaactaactgtaatatgctgctttttgttcttctcttcactgacctaagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 189 - DNA, 3570 bp, synthetic construct
  misc_feature 1..3570: note = Synthetic
  source 1..3570: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagcttggttttctctttgatccagttgacatatctgctcaccttggtgtagatgccgtacttgcccttcatggcacactcttccccccagctgatgatgcctgtcaggaagctggtgccctccacctctgtcacgtgtgggcctccagagtctccctggcagctgtctctgcctccctcatggaagccggcacagaacatgttgttgtagatggtgaacttggtgcttctcaggcaggtggctctgtccaccagggggacccgcaggtattgcagcaccagggcagacctgcccttgtggaacactctgccccagccagacacgtagccagagccaaacttcaggaagatgttggtgtactccttgtcggcaatgcagatgggggtaacatagctgttcagcaccagaggctcatcaagttccagcagggcaatgtcgtggttgtacttgttgatggcagcattgtagttgtggtgagggatgattctgatcacattccgtttctgctctgtgtgctctgtttcctcgatgttgtgctctccagccaccactgtgatcttgacacctgtctccacacagtgggcggctgtcacgatccacttttcattcacaatgctgcctccacagaaggcatccactttgccattgagcaccacctgccatgggaactggccaggcttggcatcctctccacccaccacccgggtgaagtcattgaaggactgggtgctctgggtgatgttgtccaggatggtttcagcttctgtgctgttcacgtagtccacatcagggaacactgtctcagcccgggtcagtttgctggtctggctcactgacactctgccacaggggaaaggcacggcaggttcacaggacttctggttttctgccagcctgtagccctctgtgcagctgcacaccaccttgttgtcagcagagttcttgcagaactgttcacatctgccatttttgatgttgcaggtgacatccagctcacagttcttgccttcaaagccaaaagggcaccagcattcgtagctgttgatgtcatccttgcaggagcctccattcaggcaagggttgctctcgcactggtctccatcaacgtactgcttccagaactcagtggttctttctgtgttctcgaacacttcccgggcttcctcaaagctgcacttctcttccatgcactccctctccaggttgccttgcacaaattcctccagcttgccactgttgtatctcttgggtctgttcaggatcttgttggcattctcatggtccaggaaaactgtggaaacagggagagaaaaaccacacaacatatttaaagattgatgaagacaactaactgtaatatgctgctttttgttcttctcttcactgacctaagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 190 - DNA, 3570 bp, synthetic construct
  misc_feature 1..3570: note = Synthetic
  source 1..3570: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagtttttcttgatcatgaaaatgccaacaaaattctgaataggccaaagaggtataattcagggaaattggaagagtttgttcaagggaaccttgagagagaatgtatggaagaaaagtgtagttttgaagaagcaagagaagtttttgaaaacactgaaagaacaactgaattttggaagcagtatgttgatggagatcagtgcgagtccaatccatgtttaaatgggggcagttgcaaggatgacattaattcctatgaatgttggtgtccctttggatttgaaggaaagaactgtgaattagatgtaacatgtaacattaagaatggcagatgtgagcagttttgtaaaaatagtgctgataacaaggtggtttgctcctgtactgagggatatagacttgcagaaaaccagaagtcctgtgaaccagcagtgccatttccatgtggaagagtttctgtttcacaaacttctaagctcaccagagctgagactgtttttcctgatgtggactatgtaaattctactgaagctgaaaccattttggataacatcactcaaagcacccaatcatttaatgacttcactagggttgttggtggagaagatgccaaaccaggacaattcccttggcaggttgttttgaatggtaaagttgatgcattctgtggaggctctattgttaatgaaaaatggattgtaactgctgcccactgtgttgaaactggtgttaaaattacagttgtggcaggtgaacataatattgaggagacagaacatacagagcaaaagagaaatgtgattagaattattcctcaccacaactacaatgcagctattaataagtacaaccatgacattgcccttctggaactggatgaacccttagtgctaaacagctatgttacacctatttgcattgctgacaaggaatacaccaacatcttcctcaaatttggatctggctatgtaagtggctggggaagagtcttccacaaagggagatcagctttagttcttcagtaccttagagttccacttgttgacagagccacatgtcttaggtctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatccaggtatgtcaactggattaaggaaaaaacaaagctcacttaacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagtttggttttttctttgatccagttcacatatctggacaccttggtgtagatgccatacttgcccttcatggcacattcctctccccagctgatgatgcctgtcaggaagctggtgccctccacctctgtcacgtgtgggcctccagagtctccttggcagctgtctctgcctccctcatggaagccagcacagaacatgttgttgtagatggtgaacttggtgcttctcaggcaggtggctctgtccaccagggggactctcaggtattgcagcaccagggcagacctgcccttgtggaacactctgccccagccagacacgtagccagagccaaacttcaggaagatgttggtgtactctttgtctgcaatgcagatgggggtaacatagctgttcagcaccagaggctcatcaagttccagcagggcaatgtcatggttgtacttgttgatggctgcattgtagttgtggtgagggatgatcctgatcacatttctcttctgctctgtgtgctctgtttcttctatgttgtgttcaccagccaccactgtgatcttgacacctgtttccacacagtgggcagctgtcacaatccatttttcattcacaatgctgcccccacagaaggcatccaccttgccattcagcaccacctgccatgggaactggccaggcttggcatcctctcctcccaccactctggtgaagtcattgaagctctgggtgctctgggtgatgttgtccaggatggtctcagcctcagtgctgttcacatagtccacatcagggaacactgtttcagccctggtcagcttggaggtctgggacacagacactctgccacaggggaaagggacagcaggttcacagctcttctggttttcagccagcctgtagccctctgtgcagctgcacaccaccttgttgtctgctgagttcttgcagaactgttcacatctgccattcttgatgttgcaggtgacatccagctcacagttcttgccctcaaagccaaaagggcaccagcactcatagctgttgatgtcatctttgcagctgcctccattcaggcaagggttgctctcacactggtctccatcaacgtactgcttccagaactcagtggttctctctgtgttttcaaacacttctctggcttcctcaaagctgcacttctcttccatgcactccctctccaggttgccctgcacaaactcctccagcttgccactgttgtatctcttgggtctgttgaggatcttgttggcattctcatggtccaggaaaactgtggaaacagggagagaaaaaccacacaacatatttaaagattgatgaagacaactaactgtaatatgctgctttttgttcttctcttcactgacctaagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 191 - DNA, 3570 bp, synthetic construct
  misc_feature 1..3570: note = Synthetic
  source 1..3570: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagtttttcttgatcatgaaaatgccaacaaaattctgaataggccaaagaggtataattcagggaaattggaagagtttgttcaagggaaccttgagagagaatgtatggaagaaaagtgtagttttgaagaagcaagagaagtttttgaaaacactgaaagaacaactgaattttggaagcagtatgttgatggagatcagtgcgagtccaatccatgtttaaatgggggcagttgcaaggatgacattaattcctatgaatgttggtgtccctttggatttgaaggaaagaactgtgaattagatgtaacatgtaacattaagaatggcagatgtgagcagttttgtaaaaatagtgctgataacaaggtggtttgctcctgtactgagggatatagacttgcagaaaaccagaagtcctgtgaaccagcagtgccatttccatgtggaagagtttctgtttcacaaacttctaagctcaccagagctgagactgtttttcctgatgtggactatgtaaattctactgaagctgaaaccattttggataacatcactcaaagcacccaatcatttaatgacttcactagggttgttggtggagaagatgccaaaccaggacaattcccttggcaggttgttttgaatggtaaagttgatgcattctgtggaggctctattgttaatgaaaaatggattgtaactgctgcccactgtgttgaaactggtgttaaaattacagttgtggcaggtgaacataatattgaggagacagaacatacagagcaaaagagaaatgtgattagaattattcctcaccacaactacaatgcagctattaataagtacaaccatgacattgcccttctggaactggatgaacccttagtgctaaacagctatgttacacctatttgcattgctgacaaggaatacaccaacatcttcctcaaatttggatctggctatgtaagtggctggggaagagtcttccacaaagggagatcagctttagttcttcagtaccttagagttccacttgttgacagagccacatgtcttaggtctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatccaggtatgtcaactggattaaggaaaaaacaaagctcacttaacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagcttggttttctctttgatccagttcacatatctgctaaccttggtgtagatgccatacttgcccttcatggcacactcttccccccagctgatgatgcctgtcaggaagctggtgccctccacctctgtcacatgtgggcctccagagtctccctggcagctgtctctgcctccctcatggaagccagcacagaacatgttgttgtagatggtgaacttggtgcttctcaggcaggtggccctgtccaccagggggactctcaggtattgcagtaccagggcagacctgcccttgtggaacactctgccccagccagacacatagccactgccaaacttgaggaagatgttggtgtactccttgtcagcaatgcagatgggggtaacatagctgttcagcaccagaggctcatcaagttccagcagggcaatgtcatggttgtacttgttgatggctgcattgtagttgtggtgagggatgatcctgatcacatttctcttctgctctgtgtgctctgtttcttctatgttgtgctctccagccaccactgtgattttgacacctgtctccacacagtgggcagctgtcacaatccatttctcattcacaatgctgcctccacagaaggcatccactttgccattcagcaccacctgccatgggaactggccaggcttggcatcctctccacccaccaccctggtgaagtcattgaaggactgggtgctctgggtgatgttgtccaggatggtttcagcctctgtgctgttcacatagtccacatcagggaacacagtctcagccctggtcagcttgctggtctgggacacagacactctgccacaggggaaagggacagcaggttcacaggacttctggttttctgccagcctgtagccctctgtgcagctgcacaccaccttgttgtcagcagagttcttgcagaactgttcacatctgccattcttgatgttgcaggtgacatccagctcacagtttttgccttcaaagccaaaagggcaccagcattcatagctgttgatgtcatccttgcagctgcctccattcaggcaagggttgctctcacactggtctccatccacatactgcttccagaactcagtggttctctctgtgttttcaaacacttccctggcttcttcaaagctgcacttttcttccatgcactctctctccaggttgccttgcacaaactcctccagcttgccagagttgtatctcttgggtctgttcaggatcttgttggcattctcatggtccaggaaaactgtggaaacagggagagaaaaaccacacaacatatttaaagattgatgaagacaactaactgtaatatgctgctttttgttcttctcttcactgacctaagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 192 - DNA, 3570 bp, synthetic construct
  misc_feature 1..3570: note = Synthetic
  source 1..3570: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagtttttcttgatcatgaaaatgccaacaaaattctgaataggccaaagaggtataattcagggaaattggaagagtttgttcaagggaaccttgagagagaatgtatggaagaaaagtgtagttttgaagaagcaagagaagtttttgaaaacactgaaagaacaactgaattttggaagcagtatgttgatggagatcagtgcgagtccaatccatgtttaaatgggggcagttgcaaggatgacattaattcctatgaatgttggtgtccctttggatttgaaggaaagaactgtgaattagatgtaacatgtaacattaagaatggcagatgtgagcagttttgtaaaaatagtgctgataacaaggtggtttgctcctgtactgagggatatagacttgcagaaaaccagaagtcctgtgaaccagcagtgccatttccatgtggaagagtttctgtttcacaaacttctaagctcaccagagctgagactgtttttcctgatgtggactatgtaaattctactgaagctgaaaccattttggataacatcactcaaagcacccaatcatttaatgacttcactagggttgttggtggagaagatgccaaaccaggacaattcccttggcaggttgttttgaatggtaaagttgatgcattctgtggaggctctattgttaatgaaaaatggattgtaactgctgcccactgtgttgaaactggtgttaaaattacagttgtggcaggtgaacataatattgaggagacagaacatacagagcaaaagagaaatgtgattagaattattcctcaccacaactacaatgcagctattaataagtacaaccatgacattgcccttctggaactggatgaacccttagtgctaaacagctatgttacacctatttgcattgctgacaaggaatacaccaacatcttcctcaaatttggatctggctatgtaagtggctggggaagagtcttccacaaagggagatcagctttagttcttcagtaccttagagttccacttgttgacagagccacatgtcttaggtctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatccaggtatgtcaactggattaaggaaaaaacaaagctcacttaacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagcttggttttctctttgatccagttcacatatctgctaaccttggtgtagatgccatacttgcccttcatggcacactcttccccccagctgatgatgcctgtcaggaagctggtgccctccacctctgtcacgtgtgggcctccagagtctccctggcagctgtctctgcctccctcatggaagccggcacagaacatgttgttgtagatggtgaacttggtgcttctcaggcaggtggccctgtccaccagggggactctcaggtattgcagtaccagggcagacctgcccttgtggaacactctgccccagccagacacatagccactgccaaacttgaggaagatgttggtgtactccttgtcagcaatgcagatgggggtaacatagctgttcagcaccagaggctcatcaagttccagcagggcaatgtcgtggttgtacttgttgatggctgcattgtagttgtggtgagggatgatccggatcacatttctcttctgctctgtgtgctctgtttcttctatgttgtgctctccagccaccactgtgattttgacacctgtctccacacagtgggcggctgtcacaatccatttctcattcacaatgctgcctccacagaaggcatccactttgccattcagcaccacctgccatgggaactggccaggcttggcatcctctccacccaccacccgggtgaagtcattgaaggactgggtgctctgggtgatgttgtccaggatggtttcagcctctgtgctgttcacgtagtccacatcagggaacacagtctcagccctggtcagcttgctggtctgggacacagacactctgccacaggggaaagggacagcaggttcacaggacttctggttttctgccagcctgtagccctctgtgcagctgcacaccaccttgttgtcagcagagttcttgcagaactgttcacatctgccattcttgatgttgcaggtgacatccagctcacagtttttgccttcaaagccaaaagggcaccagcattcgtagctgttgatgtcatccttgcagctgcctccattcaggcaagggttgctctcacactggtctccatccacgtactgcttccagaactcagtggttctctctgtgttttcaaacacttcccgggcttcttcaaagctgcacttttcttccatgcactctctctccaggttgccttgcacaaactcctccagcttgccagagttgtatctcttgggtctgttcaggatcttgttggcattctcatggtccaggaaaactgtggaaacagggagagaaaaaccacacaacatatttaaagattgatgaagacaactaactgtaatatgctgctttttgttcttctcttcactgacctaagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 193 - DNA, 3570 bp, synthetic construct
  misc_feature 1..3570: note = Synthetic
  source 1..3570: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagtttttcttgatcatgaaaatgccaacaaaattctgaataggccaaagaggtataattcagggaaattggaagagtttgttcaagggaaccttgagagagaatgtatggaagaaaagtgtagttttgaagaagcaagagaagtttttgaaaacactgaaagaacaactgaattttggaagcagtatgttgatggagatcagtgcgagtccaatccatgtttaaatgggggcagttgcaaggatgacattaattcctatgaatgttggtgtccctttggatttgaaggaaagaactgtgaattagatgtaacatgtaacattaagaatggcagatgtgagcagttttgtaaaaatagtgctgataacaaggtggtttgctcctgtactgagggatatagacttgcagaaaaccagaagtcctgtgaaccagcagtgccatttccatgtggaagagtttctgtttcacaaacttctaagctcaccagagctgagactgtttttcctgatgtggactatgtaaattctactgaagctgaaaccattttggataacatcactcaaagcacccaatcatttaatgacttcactagggttgttggtggagaagatgccaaaccaggacaattcccttggcaggttgttttgaatggtaaagttgatgcattctgtggaggctctattgttaatgaaaaatggattgtaactgctgcccactgtgttgaaactggtgttaaaattacagttgtggcaggtgaacataatattgaggagacagaacatacagagcaaaagagaaatgtgattagaattattcctcaccacaactacaatgcagctattaataagtacaaccatgacattgcccttctggaactggatgaacccttagtgctaaacagctatgttacacctatttgcattgctgacaaggaatacaccaacatcttcctcaaatttggatctggctatgtaagtggctggggaagagtcttccacaaagggagatcagctttagttcttcagtaccttagagttccacttgttgacagagccacatgtcttaggtctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatccaggtatgtcaactggattaaggaaaaaacaaagctcacttaacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagcttggttttctctttgatccagttgacatatctgctcaccttggtgtagatgccatacttgcccttcatggcacactcttccccccagctgatgatgcctgtcaggaagctggtgccctccacctctgtcacatgtgggcctccagagtctccctggcagctgtctctgcctccctcatggaagccagcacagaacatgttgttgtagatggtgaacttggtgcttctcaggcaggtggctctgtccaccagggggaccctcaggtattgcagcaccagggcagacctgcccttgtggaacactctgccccagccagacacatagccagagccaaacttcaggaagatgttggtgtactccttgtcagcaatgcagatgggggtaacatagctgttcagcaccagaggctcatcaagttccagcagggcaatgtcatggttgtacttgttgatggcagcattgtagttgtggtgagggatgattctgatcacattccttttctgctctgtgtgctctgtttcctcaatgttgtgctctccagccaccactgtgatcttgacacctgtctccacacagtgggcagctgtcacaatccacttttcattcacaatgctgcctccacagaaggcatccactttgccattgagcaccacctgccatgggaactggccaggcttggcatcctctccacccaccaccctggtgaagtcattgaaggactgggtgctctgggtgatgttgtccaggatggtttcagcttctgtgctgttcacatagtccacatcagggaacactgtctcagccctggtcagtttgctggtctggctcactgacactctgccacaggggaaaggcacagcaggttcacaggacttctggttttctgccagcctgtagccctctgtgcagctgcacaccaccttgttgtcagcagagttcttgcagaactgttcacatctgccatttttgatgttgcaggtgacatccagctcacagttcttgccttcaaagccaaaagggcaccagcattcatagctgttgatgtcatccttgcaggagcctccattcaggcaagggttgctctcacactggtctccatcaacatactgcttccagaactcagtggttctttctgtgttctcaaacacttccctggcttcctcaaagctgcacttctcttccatgcactccctctccaggttgccttgcacaaattcctccagcttgccactgttgtatctcttgggtctgttcaggatcttgttggcattctcatggtccaggaaaactgtggaaacagggagagaaaaaccacacaacatatttaaagattgatgaagacaactaactgtaatatgctgctttttgttcttctcttcactgacctaagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 194 - DNA, 3570 bp, synthetic construct
  misc_feature 1..3570: note = Synthetic
  source 1..3570: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctcttaggtcagtgaagagaagaacaaaaagcagcatattacagttagttgtcttcatcaatctttaaatatgttgtgtggtttttctctccctgtttccacagtttttcttgatcatgaaaatgccaacaaaattctgaataggccaaagaggtataattcagggaaattggaagagtttgttcaagggaaccttgagagagaatgtatggaagaaaagtgtagttttgaagaagcaagagaagtttttgaaaacactgaaagaacaactgaattttggaagcagtatgttgatggagatcagtgcgagtccaatccatgtttaaatgggggcagttgcaaggatgacattaattcctatgaatgttggtgtccctttggatttgaaggaaagaactgtgaattagatgtaacatgtaacattaagaatggcagatgtgagcagttttgtaaaaatagtgctgataacaaggtggtttgctcctgtactgagggatatagacttgcagaaaaccagaagtcctgtgaaccagcagtgccatttccatgtggaagagtttctgtttcacaaacttctaagctcaccagagctgagactgtttttcctgatgtggactatgtaaattctactgaagctgaaaccattttggataacatcactcaaagcacccaatcatttaatgacttcactagggttgttggtggagaagatgccaaaccaggacaattcccttggcaggttgttttgaatggtaaagttgatgcattctgtggaggctctattgttaatgaaaaatggattgtaactgctgcccactgtgttgaaactggtgttaaaattacagttgtggcaggtgaacataatattgaggagacagaacatacagagcaaaagagaaatgtgattagaattattcctcaccacaactacaatgcagctattaataagtacaaccatgacattgcccttctggaactggatgaacccttagtgctaaacagctatgttacacctatttgcattgctgacaaggaatacaccaacatcttcctcaaatttggatctggctatgtaagtggctggggaagagtcttccacaaagggagatcagctttagttcttcagtaccttagagttccacttgttgacagagccacatgtcttaggtctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatccaggtatgtcaactggattaaggaaaaaacaaagctcacttaacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagcttggttttctctttgatccagttgacatatctgctcaccttggtgtagatgccgtacttgcccttcatggcacactcttccccccagctgatgatgcctgtcaggaagctggtgccctccacctctgtcacgtgtgggcctccagagtctccctggcagctgtctctgcctccctcatggaagccggcacagaacatgttgttgtagatggtgaacttggtgcttctcaggcaggtggctctgtccaccagggggacccgcaggtattgcagcaccagggcagacctgcccttgtggaacactctgccccagccagacacgtagccagagccaaacttcaggaagatgttggtgtactccttgtcggcaatgcagatgggggtaacatagctgttcagcaccagaggctcatcaagttccagcagggcaatgtcgtggttgtacttgttgatggcagcattgtagttgtggtgagggatgattctgatcacattccgtttctgctctgtgtgctctgtttcctcgatgttgtgctctccagccaccactgtgatcttgacacctgtctccacacagtgggcggctgtcacgatccacttttcattcacaatgctgcctccacagaaggcatccactttgccattgagcaccacctgccatgggaactggccaggcttggcatcctctccacccaccacccgggtgaagtcattgaaggactgggtgctctgggtgatgttgtccaggatggtttcagcttctgtgctgttcacgtagtccacatcagggaacactgtctcagcccgggtcagtttgctggtctggctcactgacactctgccacaggggaaaggcacggcaggttcacaggacttctggttttctgccagcctgtagccctctgtgcagctgcacaccaccttgttgtcagcagagttcttgcagaactgttcacatctgccatttttgatgttgcaggtgacatccagctcacagttcttgccttcaaagccaaaagggcaccagcattcgtagctgttgatgtcatccttgcaggagcctccattcaggcaagggttgctctcgcactggtctccatcaacgtactgcttccagaactcagtggttctttctgtgttctcgaacacttcccgggcttcctcaaagctgcacttctcttccatgcactccctctccaggttgccttgcacaaattcctccagcttgccactgttgtatctcttgggtctgttcaggatcttgttggcattctcatggtccaggaaaactgtggaaacagggagagaaaaaccacacaacatatttaaagattgatgaagacaactaactgtaatatgctgctttttgttcttctcttcactgacctaagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 195 - protein, 431 aa, synthetic construct
  REGION 1..431: note = Synthetic
FLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 196 - DNA, 145 bp, synthetic construct
  misc_feature 1..145: note = Synthetic
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 197 - DNA, 141 bp, synthetic construct
  misc_feature 1..141: note = Synthetic
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 198 - DNA, 130 bp, synthetic construct
  misc_feature 1..130: note = Synthetic
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 199 - DNA, 131 bp, synthetic construct
  misc_feature 1..131: note = Synthetic
  source 1..131: mol_type = other DNA
cagacatgataagatacattgatgagtttggacaaaccacaactagaatgcagtgaaaaaaatgctttatttgtgaaatttgtgatgctattgctttatttgtaaccattataagctgcaataaacaagtt

SEQ ID NO: 200 - DNA, 224 bp, synthetic construct
  misc_feature 1..224: note = Synthetic
  source 1..224: mol_type = other DNA
ctgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatg

SEQ ID NO: 201 - DNA, 100 bp, synthetic construct
  misc_feature 1..100: note = Synthetic
  source 1..100: mol_type = other DNA
taggtcagtgaagagaagaacaaaaagcagcatattacagttagttgtcttcatcaatctttaaatatgttgtgtggtttttctctccctgtttccacag

SEQ ID NO: 202 - DNA, 3280 bp, synthetic construct
  misc_feature 1..3280: note = Synthetic
  misc_feature 9..108: note = Splice acceptor
  misc_feature 111..1406: note = F9 wildtype
  misc_feature 1410..1637: note = bGH pA
  misc_feature 1744..1874: note = SV40 pA
  misc_feature 1875..3170: note = F9 codon optimized
  misc_feature 3173..3272: note = Splice acceptor
  source 1..3280: mol_type = other DNA
agatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgttaggtgagcttagtcttttcttttatccaattcacgtagcgagagaccttcgtatagatgccatatttccccttcatcgcacattcctccccccaacttattatcccggtcaagaaacttgttccttcgacttcagtgacgtgtggtccacctgaatcaccttggcatgagtcgcgaccgccctcgtgaaacccagcacaaaacatgttattgtaaatcgtaaatttcgtggaccgaagacaggtcgctctatcgaccaacgggacgcgcaaatattgcagaacgagggctgatcgacctttgtggaagacccgcccccacccactcacatatccgctcccaaatttcaagaagatatttgtatattctttatcggctatacaaatcggggtaacataggagttaagtacgagtggctcgtccagctccaggagggctatatcatggttgtacttgtttatagcggcattataattgtgatggggtatgatcctgataacattccttttctgttcagtatgctcagtttcttcaatgttgtgttcgccagccacgaccgtaatcttaacccccgtctcgacacagtgtgcggccgttacaatccacttttcattgactatggagcccccacaaaacgcgtcgacttttccgttgagcaccacctgccatggaaattggccaggtttagcgtcctcgcccccgacaaccctagtaaagtcattaaatgactgtgtggattgtgttatattatcaagaatcgtttcggcttcagtagagttaacgtagtccacatcgggaaaaactgtctcggcccttgtcaactttgatgtctgggacacacttacccgaccgcacgggaagggcaccgccggttcacagctcttttgattctcagcgagccggtagccctcagtgcaactacacacaactttgttgtcggcggaatttttacagaattgctcgcatcgtccatttttaatgttgcaggtgacgtccaactcgcagttttttccttcaaaaccaaaagggcaccaacactcgtaggaatttatatcgtctttacaactccccccattcagacatggattagattcgcattggtccccatcgacatattgcttccagaactcagtggtccgttctgtattctcaaacacctcgcgcgcttcttcaaaactgcatttttcctccatacactctcgctccaagttcccttgcacgaattcttcaagctttcctgagttataccttttaggccggttaagtatcttattcgcgttttcgtggtccagaaaaactgtggaaacagggagagaaaaaccacacaacatatttaaagattgatgaagacaactaactgtaatatgctgctttttgttcttctcttcactgacctaagagatct

SEQ ID NO: 203 - DNA, 3280 bp, synthetic construct
  misc_feature 1..3280: note = Synthetic
  source 1..3280: mol_type = other DNA
agatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaagtcagcttggttttctccttaatccagttcacatatctggatacttttgtgtagatcccatactttcccttcatagcacactcctcaccccagctgataatcccagtcaggaagctggttccctccacctcagtcacatggggacccccagaatctccctggcagctgtctctaccaccttcatggaaccctgcacagaacatattgttgtagattgtaaacttggtgctcctcaagcaggttgctctatccaccagaggcaccctcaaatactgcaataccagggcagacctgcctttatggaacaccctgccccagccagacacatatccagacccaaatttcaagaagatgttggtgtactctttatcagcaatacagatgggtgtcacatagctgttcaagaccaggggttcatccagttccaggagggcaatgtcatggttgtacttgttgatagcagcattgtagttgtggtgggggataatcctgatcacattcctcttctgctcagtgtgctcagtttcctcaatattgtgctctccagccaccacagtaatcttgactccagtttccacacagtgggctgcagtcacaatccacttctcatttacaattgaacctccacaaaaggcatccacttttccattgagcactacctgccaggggaactgcccaggcttggcatcctcccctcctaccaccctggtgaagtcattgaagctctgtgtgctctgagtgatattgtcaaggattgtctcagcctcagtgctattcacataatctacatcaggaaagactgtttcagccctagtcagcttgctggtttggctcacagacactctcccacagggaaaaggcactgcaggttcacagctcttctggttttcagccagcctatatccctcagtgcagctacagaccactttgttatcagcagaattcttgcaaaattgctcacacctgccattcttgatattacaggtcacatcaagctcacagtttttcccctcaaatccaaagggacaccaacattcatagctgttgatatcatctttacagctccccccattcaagcagggattgctctcacattgatcaccatccacatactgcttccaaaattcagttgttctctctgtattttcaaacacttctctggcttcctcaaagctgcacttctcttccatacactctctctccagattcccctgtacaaactcttcaagcttgccagagttatacctctttggtctattcagaattttgttggcattttcatgatcaagaaaaactgtggaaacagggagagaaaaaccacacaacatatttaaagattgatgaagacaactaactgtaatatgctgctttttgttcttctcttcactgacctaagagatct

SEQ ID NO: 204 - DNA, 3280 bp, synthetic construct
  misc_feature 1..3280: note = Synthetic
  source 1..3280: mol_type = other DNA
agatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagcttggtcttctccttgatccagttcacatacctgctcaccttggtgtagatgccatacttgcccttcatggcacactcctccccccagctgatgatgcctgtcaggaagctggtgccctccacctctgtcacatgggggcccccagagtctccctggcagctgtctctgcctccctcatggaagccagcacagaacatgttgttgtagatggtgaacttggtgctcctcaggcaggtggccctgtccaccaggggcactctcaggtactgcagcaccagggcagacctgcccttgtggaacactctgccccagccagacacatagccagagccaaacttcaggaagatgttggtgtactccttgtcagcaatgcagatgggggtcacatagctgttcagcaccaggggctcatccagctccagcagggcaatgtcatggttgtacttgttgatggcagcattgtagttgtggtgggggatgatcctgatcacattcctcttctgctctgtgtgctctgtctcctcaatgttgtgctccccagccaccactgtgatcttcacccctgtctccacacagtgggcagctgtcacaatccacttctcattcacaatgctgcctccacagaaggcatccaccttgccattcagcaccacctgccaggggaactggccaggcttggcatcctctccccccaccaccctggtgaagtcattgaagctctgggtgctctgggtgatgttgtccaggatggtctcagcctctgtgctgttcacatagtccacatcagggaacactgtctcagccctggtcagcttgctggtctggctcacagacactctgccacaggggaagggcacagcaggctcacagctcttctggttctcagccagcctgtagccctctgtgcagctgcacaccaccttgttgtcagcagagttcttgcagaactgctcacatctgccattcttgatgttgcaggtcacatccagctcacagttcttgccctcaaagccaaaggggcaccagcactcatagctgttgatgtcatccttgcagctgcctccattcaggcaggggttgctctcacactggtctccatccacatactgcttccagaactctgtggttctctctgtgttctcaaacacctccctggcctcctcaaagctgcacttctcctccatgcactctctctccaggttgccctgcacaaactcctccagcttgccagagttgtacctcttgggcctgttcaggatcttgttggcattctcatggtccaggaaaactgtggaaacagggagagaaaaaccacacaacatatttaaagattgatgaagacaactaactgtaatatgctgctttttgttcttctcttcactgacctaagagatct

SEQ ID NO: 205 - DNA, 3280 bp, synthetic construct
  misc_feature 1..3280: note = Synthetic
  source 1..3280: mol_type = other DNA
agatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtgagcttggtcttttctttgatccagttgacatatctggacaccttggtgtagatgccatatttgcccttcatggcacactcttctccccagctgatgatgcctgtcagaaagctggtgccttccacctctgtcacatgagggccaccactatctccctggcaggagtctctgccaccttcatggaacccagcacagaacatgttgttgtagatggtgaacttggtgcttctcagacaggtggctctatccaccaggggcactctcaggtactgcagcaccagggcagaccttcccttgtggaacactctaccccagccagacacatagccagagccaaacttcagaaagatgttggtgtactctttgtcagcaatgcagataggggtcacataggagttcaggaccaggggctcatccagctcaagcagggcaatgtcatggttgtacttgttgatggcagcattgtagttgtggtgagggatgatcctgatgacattcctcttttgctctgtgtgctctgtttcctcaatgttgtgctccccagccaccactgtgatcttgacccctgtttccacacagtgggcagctgtcacaatccacttctcattcacaatggagccaccacagaaggcatccactttgccattcagcaccacttgccaagggaactgtccaggcttggcatcttcccctccaacaactcttgtgaagtcattgaaggactgggtgctctgggtgatgttgtccaggatggtttcagcctctgtggagttcacatagtccacatcagggaacactgtctcagctctggtcagcttgctggtctgggacacagacactctgccacatggaaagggcacagcaggttcacagcttttctggttctcagccagtctgtagccctctgtgcaggagcacacaaccttgttgtcagcagagttcttgcagaactgttcacatctgccattcttgatgttgcaggtcacatccagttcacagttcttgccctcaaagccaaaggggcaccagcactcatagctgttgatgtcatccttgcagctgccaccattcaggcaggggttgctttcacactggtccccatccacatactgcttccagaactctgtggttctctctgtgttctcaaacacttccctggcctcttcaaagctgcacttctcttccatgcattccctttccaggttgccctgcacaaactcttccagcttgccagagttgtacctcttgggcctgttcaggatcttgttggcattctcatggtccaggaaaactgtggaaacagggagagaaaaaccacacaacatatttaaagattgatgaagacaactaactgtaatatgctgctttttgttcttctcttcactgacctaagagatct

SEQ ID NO: 206 - DNA, 3280 bp, synthetic construct
  misc_feature 1..3280: note = Synthetic
  source 1..3280: mol_type = other DNA
agatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagtttggttttttctttgatccagttcacatatctggacaccttggtgtagatgccatacttgcccttcatggcacattcctctccccagctgatgatgcctgtcaggaagctggtgccctccacctctgtcacatgtgggcctccagagtctccttggcagctgtctctgcctccctcatggaagccagcacagaacatgttgttgtagatggtgaacttggtgcttctcaggcaggtggctctgtccaccagggggactctcaggtattgcagcaccagggcagacctgcccttgtggaacactctgccccagccagacacatagccagagccaaacttcaggaagatgttggtgtactctttgtctgcaatgcagatgggggtaacatagctgttcagcaccagaggctcatcaagttccagcagggcaatgtcatggttgtacttgttgatggctgcattgtagttgtggtgagggatgatcctgatcacatttctcttctgctctgtgtgctctgtttcttctatgttgtgttcaccagccaccactgtgatcttgacacctgtttccacacagtgggcagctgtcacaatccatttttcattcacaatgctgcccccacagaaggcatccaccttgccattcagcaccacctgccatgggaactggccaggcttggcatcctctcctcccaccactctggtgaagtcattgaagctctgggtgctctgggtgatgttgtccaggatggtctcagcctcagtgctgttcacatagtccacatcagggaacactgtttcagccctggtcagcttggaggtctgggacacagacactctgccacaggggaaagggacagcaggttcacagctcttctggttttcagccagcctgtagccctctgtgcagctgcacaccaccttgttgtctgctgagttcttgcagaactgttcacatctgccattcttgatgttgcaggtgacatccagctcacagttcttgccctcaaagccaaaagggcaccagcactcatagctgttgatgtcatctttgcagctgcctccattcaggcaagggttgctctcacactggtctccatcaacatactgcttccagaactcagtggttctctctgtgttttcaaacacttctctggcttcctcaaagctgcacttctcttccatgcactccctctccaggttgccctgcacaaactcctccagcttgccactgttgtatctcttgggtctgttgaggatcttgttggcattctcatggtccaggaaaactgtggaaacagggagagaaaaaccacacaacatatttaaagattgatgaagacaactaactgtaatatgctgctttttgttcttctcttcactgacctaagagatct

SEQ ID NO: 207 - DNA, 3280 bp, synthetic construct
  misc_feature 1..3280: note = Synthetic
  source 1..3280: mol_type = other DNA
agatctcttaggtcagtgaagagaagaacaaaaagcagcatattacagttagttgtcttcatcaatctttaaatatgttgtgtggtttttctctccctgtttccacagtttttcttgatcatgaaaatgccaacaaaattctgaataggccaaagaggtataattcagggaaattggaagagtttgttcaagggaaccttgagagagaatgtatggaagaaaagtgtagttttgaagaagcaagagaagtttttgaaaacactgaaagaacaactgaattttggaagcagtatgttgatggagatcagtgcgagtccaatccatgtttaaatgggggcagttgcaaggatgacattaattcctatgaatgttggtgtccctttggatttgaaggaaagaactgtgaattagatgtaacatgtaacattaagaatggcagatgtgagcagttttgtaaaaatagtgctgataacaaggtggtttgctcctgtactgagggatatagacttgcagaaaaccagaagtcctgtgaaccagcagtgccatttccatgtggaagagtttctgtttcacaaacttctaagctcaccagagctgagactgtttttcctgatgtggactatgtaaattctactgaagctgaaaccattttggataacatcactcaaagcacccaatcatttaatgacttcactagggttgttggtggagaagatgccaaaccaggacaattcccttggcaggttgttttgaatggtaaagttgatgcattctgtggaggctctattgttaatgaaaaatggattgtaactgctgcccactgtgttgaaactggtgttaaaattacagttgtggcaggtgaacataatattgaggagacagaacatacagagcaaaagagaaatgtgattagaattattcctcaccacaactacaatgcagctattaataagtacaaccatgacattgcccttctggaactggatgaacccttagtgctaaacagctatgttacacctatttgcattgctgacaaggaatacaccaacatcttcctcaaatttggatctggctatgtaagtggctggggaagagtcttccacaaagggagatcagctttagttcttcagtaccttagagttccacttgttgacagagccacatgtcttaggtctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatccaggtatgtcaactggattaaggaaaaaacaaagctcacttaacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaagtcagcttggttttctccttaatccagttcacatatctggatacttttgtgtagatcccatactttcccttcatagcacactcctcaccccagctgataatcccagtcaggaagctggttccctccacctcagtcacatggggacccccagaatctccctggcagctgtctctaccaccttcatggaaccctgcacagaacatattgttgtagattgtaaacttggtgctcctcaagcaggttgctctatccaccagaggcaccctcaaatactgcaataccagggcagacctgcctttatggaacaccctgccccagccagacacatatccagacccaaatttcaagaagatgttggtgtactctttatcagcaatacagatgggtgtcacatagctgttcaagaccaggggttcatccagttccaggagggcaatgtcatggttgtacttgttgatagcagcattgtagttgtggtgggggataatcctgatcacattcctcttctgctcagtgtgctcagtttcctcaatattgtgctctccagccaccacagtaatcttgactccagtttccacacagtgggctgcagtcacaatccacttctcatttacaattgaacctccacaaaaggcatccacttttccattgagcactacctgccaggggaactgcccaggcttggcatcctcccctcctaccaccctggtgaagtcattgaagctctgtgtgctctgagtgatattgtcaaggattgtctcagcctcagtgctattcacataatctacatcaggaaagactgtttcagccctagtcagcttgctggtttggctcacagacactctcccacagggaaaaggcactgcaggttcacagctcttctggttttcagccagcctatatccctcagtgcagctacagaccactttgttatcagcagaattcttgcaaaattgctcacacctgccattcttgatattacaggtcacatcaagctcacagtttttcccctcaaatccaaagggacaccaacattcatagctgttgatatcatctttacagctccccccattcaagcagggattgctctcacattgatcaccatccacatactgcttccaaaattcagttgttctctctgtattttcaaacacttctctggcttcctcaaagctgcacttctcttccatacactctctctccagattcccctgtacaaactcttcaagcttgccagagttatacctctttggtctattcagaattttgttggcattttcatgatcaagaaaaactgtggaaacagggagagaaaaaccacacaacatatttaaagattgatgaagacaactaactgtaatatgctgctttttgttcttctcttcactgacctaagagatct

SEQ ID NO: 208 - DNA, 3280 bp, synthetic construct
  misc_feature 1..3280: note = Synthetic
  source 1..3280: mol_type = other DNA
agatctcttaggtcagtgaagagaagaacaaaaagcagcatattacagttagttgtcttcatcaatctttaaatatgttgtgtggtttttctctccctgtttccacagtttttcttgatcatgaaaatgccaacaaaattctgaataggccaaagaggtataattcagggaaattggaagagtttgttcaagggaaccttgagagagaatgtatggaagaaaagtgtagttttgaagaagcaagagaagtttttgaaaacactgaaagaacaactgaattttggaagcagtatgttgatggagatcagtgcgagtccaatccatgtttaaatgggggcagttgcaaggatgacattaattcctatgaatgttggtgtccctttggatttgaaggaaagaactgtgaattagatgtaacatgtaacattaagaatggcagatgtgagcagttttgtaaaaatagtgctgataacaaggtggtttgctcctgtactgagggatatagacttgcagaaaaccagaagtcctgtgaaccagcagtgccatttccatgtggaagagtttctgtttcacaaacttctaagctcaccagagctgagactgtttttcctgatgtggactatgtaaattctactgaagctgaaaccattttggataacatcactcaaagcacccaatcatttaatgacttcactagggttgttggtggagaagatgccaaaccaggacaattcccttggcaggttgttttgaatggtaaagttgatgcattctgtggaggctctattgttaatgaaaaatggattgtaactgctgcccactgtgttgaaactggtgttaaaattacagttgtggcaggtgaacataatattgaggagacagaacatacagagcaaaagagaaatgtgattagaattattcctcaccacaactacaatgcagctattaataagtacaaccatgacattgcccttctggaactggatgaacccttagtgctaaacagctatgttacacctatttgcattgctgacaaggaatacaccaacatcttcctcaaatttggatctggctatgtaagtggctggggaagagtcttccacaaagggagatcagctttagttcttcagtaccttagagttccacttgttgacagagccacatgtcttaggtctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatccaggtatgtcaactggattaaggaaaaaacaaagctcacttaacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagcttggtcttctccttgatccagttcacatacctgctcaccttggtgtagatgccatacttgcccttcatggcacactcctccccccagctgatgatgcctgtcaggaagctggtgccctccacctctgtcacatgggggcccccagagtctccctggcagctgtctctgcctccctcatggaagccagcacagaacatgttgttgtagatggtgaacttggtgctcctcaggcaggtggccctgtccaccaggggcactctcaggtactgcagcaccagggcagacctgcccttgtggaacactctgccccagccagacacatagccagagccaaacttcaggaagatgttggtgtactccttgtcagcaatgcagatgggggtcacatagctgttcagcaccaggggctcatccagctccagcagggcaatgtcatggttgtacttgttgatggcagcattgtagttgtggtgggggatgatcctgatcacattcctcttctgctctgtgtgctctgtctcctcaatgttgtgctccccagccaccactgtgatcttcacccctgtctccacacagtgggcagctgtcacaatccacttctcattcacaatgctgcctccacagaaggcatccaccttgccattcagcaccacctgccaggggaactggccaggcttggcatcctctccccccaccaccctggtgaagtcattgaagctctgggtgctctgggtgatgttgtccaggatggtctcagcctctgtgctgttcacatagtccacatcagggaacactgtctcagccctggtcagcttgctggtctggctcacagacactctgccacaggggaagggcacagcaggctcacagctcttctggttctcagccagcctgtagccctctgtgcagctgcacaccaccttgttgtcagcagagttcttgcagaactgctcacatctgccattcttgatgttgcaggtcacatccagctcacagttcttgccctcaaagccaaaggggcaccagcactcatagctgttgatgtcatccttgcagctgcctccattcaggcaggggttgctctcacactggtctccatccacatactgcttccagaactctgtggttctctctgtgttctcaaacacctccctggcctcctcaaagctgcacttctcctccatgcactctctctccaggttgccctgcacaaactcctccagcttgccagagttgtacctcttgggcctgttcaggatcttgttggcattctcatggtccaggaaaactgtggaaacagggagagaaaaaccacacaacatatttaaagattgatgaagacaactaactgtaatatgctgctttttgttcttctcttcactgacctaagagatct

SEQ ID NO: 209 - DNA, 3280 bp, synthetic construct
  misc_feature 1..3280: note = Synthetic
  misc_feature 9..108: note = Splice acceptor
  misc_feature 111..1406: note = F9 CDS #1 Native CpG Removed No Splice F9
  misc_feature 170: note = Cryptic splice donor site mutated
  misc_feature 311: note = Cryptic splice donor site mutated
  misc_feature 728: note = Cryptic splice donor site mutated
  misc_feature 1410..1411: note = CpG removed
  misc_feature 1413..1636: note = bGH pA with CpG sites removed
  misc_feature 1647..1648: note = CpG removed
  misc_feature 1744..1874: note = SV40 pA
  misc_feature 1875..3170: note = F9 CDS #2 (GA F9)
  misc_feature 3173..3272: note = Splice acceptor
  source 1..3280: mol_type = other DNA
agatctcttaggtcagtgaagagaagaacaaaaagcagcatattacagttagttgtcttcatcaatctttaaatatgttgtgtggtttttctctccctgtttccacagtttttcttgatcatgaaaatgccaacaaaattctgaataggccaaagaggtataattcagggaaattggaagagtttgttcaagggaaccttgagagagaatgtatggaagaaaagtgtagttttgaagaagcaagagaagtttttgaaaacactgaaagaacaactgaattttggaagcagtatgttgatggagatcagtgcgagtccaatccatgtttaaatgggggcagttgcaaggatgacattaattcctatgaatgttggtgtccctttggatttgaaggaaagaactgtgaattagatgtaacatgtaacattaagaatggcagatgtgagcagttttgtaaaaatagtgctgataacaaggtggtttgctcctgtactgagggatatagacttgcagaaaaccagaagtcctgtgaaccagcagtgccatttccatgtggaagagtttctgtttcacaaacttctaagctcaccagagctgagactgtttttcctgatgtggactatgtaaattctactgaagctgaaaccattttggataacatcactcaaagcacccaatcatttaatgacttcactagggttgttggtggagaagatgccaaaccaggacaattcccttggcaggttgttttgaatggtaaagttgatgcattctgtggaggctctattgttaatgaaaaatggattgtaactgctgcccactgtgttgaaactggtgttaaaattacagttgtggcaggtgaacataatattgaggagacagaacatacagagcaaaagagaaatgtgattagaattattcctcaccacaactacaatgcagctattaataagtacaaccatgacattgcccttctggaactggatgaacccttagtgctaaacagctatgttacacctatttgcattgctgacaaggaatacaccaacatcttcctcaaatttggatctggctatgtaagtggctggggaagagtcttccacaaagggagatcagctttagttcttcagtaccttagagttccacttgttgacagagccacatgtcttaggtctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatccaggtatgtcaactggattaaggaaaaaacaaagctcacttaacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtgagcttggtcttttctttgatccagttgacatatctggacaccttggtgtagatgccatatttgcccttcatggcacactcttctccccagctgatgatgcctgtcagaaagctggtgccttccacctctgtcacatgagggccaccactatctccctggcaggagtctctgccaccttcatggaacccagcacagaacatgttgttgtagatggtgaacttggtgcttctcagacaggtggctctatccaccaggggcactctcaggtactgcagcaccagggcagaccttcccttgtggaacactctaccccagccagacacatagccagagccaaacttcagaaagatgttggtgtactctttgtcagcaatgcagataggggtcacataggagttcaggaccaggggctcatccagctcaagcagggcaatgtcatggttgtacttgttgatggcagcattgtagttgtggtgagggatgatcctgatgacattcctcttttgctctgtgtgctctgtttcctcaatgttgtgctccccagccaccactgtgatcttgacccctgtttccacacagtgggcagctgtcacaatccacttctcattcacaatggagccaccacagaaggcatccactttgccattcagcaccacttgccaagggaactgtccaggcttggcatcttcccctccaacaactcttgtgaagtcattgaaggactgggtgctctgggtgatgttgtccaggatggtttcagcctctgtggagttcacatagtccacatcagggaacactgtctcagctctggtcagcttgctggtctgggacacagacactctgccacatggaaagggcacagcaggttcacagcttttctggttctcagccagtctgtagccctctgtgcaggagcacacaaccttgttgtcagcagagttcttgcagaactgttcacatctgccattcttgatgttgcaggtcacatccagttcacagttcttgccctcaaagccaaaggggcaccagcactcatagctgttgatgtcatccttgcagctgccaccattcaggcaggggttgctttcacactggtccccatccacatactgcttccagaactctgtggttctctctgtgttctcaaacacttccctggcctcttcaaagctgcacttctcttccatgcattccctttccaggttgccctgcacaaactcttccagcttgccagagttgtacctcttgggcctgttcaggatcttgttggcattctcatggtccaggaaaactgtggaaacagggagagaaaaaccacacaacatatttaaagattgatgaagacaactaactgtaatatgctgctttttgttcttctcttcactgacctaagagatct

SEQ ID NO: 210 - DNA, 3280 bp, synthetic construct
  misc_feature 1..3280: note = Synthetic
  misc_feature 9..108: note = Splice acceptor
  misc_feature 111..1406: note = F9 CDS #1 Native CpG Removed No Splice F9
  misc_feature 170: note = Cryptic splice donor site mutated
  misc_feature 311: note = Cryptic splice donor site mutated
  misc_feature 728: note = Cryptic splice donor site mutated
  misc_feature 1410..1411: note = CpG removed
  misc_feature 1413..1636: note = bGH pA with CpG sites removed
  misc_feature 1647..1648: note = CpG removed
  misc_feature 1744..1874: note = SV40 pA
  misc_feature 1875..3170: note = F9 CDS #2 (CpG0 F9)
  misc_feature 3173..3272: note = Splice acceptor
  source 1..3280: mol_type = other DNA
agatctcttaggtcagtgaagagaagaacaaaaagcagcatattacagttagttgtcttcatcaatctttaaatatgttgtgtggtttttctctccctgtttccacagtttttcttgatcatgaaaatgccaacaaaattctgaataggccaaagaggtataattcagggaaattggaagagtttgttcaagggaaccttgagagagaatgtatggaagaaaagtgtagttttgaagaagcaagagaagtttttgaaaacactgaaagaacaactgaattttggaagcagtatgttgatggagatcagtgcgagtccaatccatgtttaaatgggggcagttgcaaggatgacattaattcctatgaatgttggtgtccctttggatttgaaggaaagaactgtgaattagatgtaacatgtaacattaagaatggcagatgtgagcagttttgtaaaaatagtgctgataacaaggtggtttgctcctgtactgagggatatagacttgcagaaaaccagaagtcctgtgaaccagcagtgccatttccatgtggaagagtttctgtttcacaaacttctaagctcaccagagctgagactgtttttcctgatgtggactatgtaaattctactgaagctgaaaccattttggataacatcactcaaagcacccaatcatttaatgacttcactagggttgttggtggagaagatgccaaaccaggacaattcccttggcaggttgttttgaatggtaaagttgatgcattctgtggaggctctattgttaatgaaaaatggattgtaactgctgcccactgtgttgaaactggtgttaaaattacagttgtggcaggtgaacataatattgaggagacagaacatacagagcaaaagagaaatgtgattagaattattcctcaccacaactacaatgcagctattaataagtacaaccatgacattgcccttctggaactggatgaacccttagtgctaaacagctatgttacacctatttgcattgctgacaaggaatacaccaacatcttcctcaaatttggatctggctatgtaagtggctggggaagagtcttccacaaagggagatcagctttagttcttcagtaccttagagttccacttgttgacagagccacatgtcttaggtctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatccaggtatgtcaactggattaaggaaaaaacaaagctcacttaacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagtttggttttttctttgatccagttcacatatctggacaccttggtgtagatgccatacttgcccttcatggcacattcctctccccagctgatgatgcctgtcaggaagctggtgccctccacctctgtcacatgtgggcctccagagtctccttggcagctgtctctgcctccctcatggaagccagcacagaacatgttgttgtagatggtgaacttggtgcttctcaggcaggtggctctgtccaccagggggactctcaggtattgcagcaccagggcagacctgcccttgtggaacactctgccccagccagacacatagccagagccaaacttcaggaagatgttggtgtactctttgtctgcaatgcagatgggggtaacatagctgttcagcaccagaggctcatcaagttccagcagggcaatgtcatggttgtacttgttgatggctgcattgtagttgtggtgagggatgatcctgatcacatttctcttctgctctgtgtgctctgtttcttctatgttgtgttcaccagccaccactgtgatcttgacacctgtttccacacagtgggcagctgtcacaatccatttttcattcacaatgctgcccccacagaaggcatccaccttgccattcagcaccacctgccatgggaactggccaggcttggcatcctctcctcccaccactctggtgaagtcattgaagctctgggtgctctgggtgatgttgtccaggatggtctcagcctcagtgctgttcacatagtccacatcagggaacactgtttcagccctggtcagcttggaggtctgggacacagacactctgccacaggggaaagggacagcaggttcacagctcttctggttttcagccagcctgtagccctctgtgcagctgcacaccaccttgttgtctgctgagttcttgcagaactgttcacatctgccattcttgatgttgcaggtgacatccagctcacagttcttgccctcaaagccaaaagggcaccagcactcatagctgttgatgtcatctttgcagctgcctccattcaggcaagggttgctctcacactggtctccatcaacatactgcttccagaactcagtggttctctctgtgttttcaaacacttctctggcttcctcaaagctgcacttctcttccatgcactccctctccaggttgccctgcacaaactcctccagcttgccactgttgtatctcttgggtctgttgaggatcttgttggcattctcatggtccaggaaaactgtggaaacagggagagaaaaaccacacaacatatttaaagattgatgaagacaactaactgtaatatgctgctttttgttcttctcttcactgacctaagagatct

SEQ ID NO: 211 - DNA, 3280 bp, synthetic construct
  misc_feature 1..3280: note = Synthetic
  source 1..3280: mol_type = other DNA
agatctcttaggtcagtgaagagaagaacaaaaagcagcatattacagttagttgtcttcatcaatctttaaatatgttgtgtggtttttctctccctgtttccacagtttttcttgatcatgaaaatgccaacaaaattctgaataggccaaagaggtataattcagggaaattggaagagtttgttcaagggaaccttgagagagaatgtatggaagaaaagtgtagttttgaagaagcaagagaagtttttgaaaacactgaaagaacaactgaattttggaagcagtatgttgatggagatcagtgcgagtccaatccatgtttaaatgggggcagttgcaaggatgacattaattcctatgaatgttggtgtccctttggatttgaaggaaagaactgtgaattagatgtaacatgtaacattaagaatggcagatgtgagcagttttgtaaaaatagtgctgataacaaggtggtttgctcctgtactgagggatatagacttgcagaaaaccagaagtcctgtgaaccagcagtgccatttccatgtggaagagtttctgtttcacaaacttctaagctcaccagagctgagactgtttttcctgatgtggactatgtaaattctactgaagctgaaaccattttggataacatcactcaaagcacccaatcatttaatgacttcactagggttgttggtggagaagatgccaaaccaggacaattcccttggcaggttgttttgaatggtaaagttgatgcattctgtggaggctctattgttaatgaaaaatggattgtaactgctgcccactgtgttgaaactggtgttaaaattacagttgtggcaggtgaacataatattgaggagacagaacatacagagcaaaagagaaatgtgattagaattattcctcaccacaactacaatgcagctattaataagtacaaccatgacattgcccttctggaactggatgaacccttagtgctaaacagctatgttacacctatttgcattgctgacaaggaatacaccaacatcttcctcaaatttggatctggctatgtaagtggctggggaagagtcttccacaaagggagatcagctttagttcttcagtaccttagagttccacttgttgacagagccacatgtcttaggtctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatccaggtatgtcaactggattaaggaaaaaacaaagctcacttaacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgttaggtgagcttagtcttttcttttatccaattcacgtagcgagagaccttcgtatagatgccatatttccccttcatcgcacattcctccccccaacttattatcccggtcaagaaacttgttccttcgacttcagtgacgtgtggtccacctgaatcaccttggcatgagtcgcgaccgccctcgtgaaacccagcacaaaacatgttattgtaaatcgtaaatttcgtggaccgaagacaggtcgctctatcgaccaacgggacgcgcaaatattgcagaacgagggctgaccgacctttgtggaagacccgcccccacccactcacatatccgctcccaaatttcaagaagatatttgtatattctttatcggctatacaaatcggggtaacataggagttaagtacgagtggctcgtccagctccaggagggctatatcatggttgtacttgtttatagcggcattataattgtgatggggtatgatcctgataacattccttttctgttcagtatgctcagtttcttcaatgttgtgttcgccagccacgaccgtaatcttaacccccgtctcgacacagtgtgcggccgttacaatccacttttcattgactatggagcccccacaaaacgcgtcgacttttccgttgagcaccacctgccatggaaattggccaggtttagcgtcctcgcccccgacaaccctagtaaagtcattaaatgactgtgtggattgtgttatattatcaagaatcgtttcggcttcagtagagttaacgtagtccacatcgggaaaaactgtctcggcccttgtcaactttgatgtctgggacacacttacccgaccgcacgggaagggcaccgccggttcacagctcttttgattctcagcgagccggtagccctcagtgcaactacacacaactttgttgtcggcggaatttttacagaattgctcgcatcgtccatttttaatgttgcaggtgacgtccaactcgcagttttttccttcaaaaccaaaagggcaccaacactcgtaggaatttatatcgtctttacaactccccccattcagacatggattagattcgcattggtccccatcgacatattgcttccagaactcagtggtccgttctgtattctcaaacacctcgcgcgcttcttcaaaactgcatttttcctccatacactctcgctccaagttcccttgcacgaattcttcaagctttcctgagttataccttttaggccggttaagtatcttattcgcgttttcgtggtccagaaaaactgtggaaacagggagagaaaaaccacacaacatatttaaagattgatgaagacaactaactgtaatatgctgctttttgttcttctcttcactgacctaagagatct

SEQ ID NO: 212 - DNA, 3280 bp, synthetic construct
  misc_feature 1..3280: note = Synthetic
  source 1..3280: mol_type = other DNA
agatctcttaggtcagtgaagagaagaacaaaaagcagcatattacagttagttgtcttcatcaatctttaaatatgttgtgtggtttttctctccctgtttccacagtttttcttgatcatgaaaatgccaacaaaattctgaataggccaaagaggtataattcaggtaaattggaagagtttgttcaagggaaccttgagagagaatgtatggaagaaaagtgtagttttgaagaagcaagagaagtttttgaaaacactgaaagaacaactgaattttggaagcagtatgttgatggagatcagtgtgagtccaatccatgtttaaatgggggcagttgcaaggatgacattaattcctatgaatgttggtgtccctttggatttgaaggaaagaactgtgaattagatgtaacatgtaacattaagaatggcagatgtgagcagttttgtaaaaatagtgctgataacaaggtggtttgctcctgtactgagggatatagacttgcagaaaaccagaagtcctgtgaaccagcagtgccatttccatgtggaagagtttctgtttcacaaacttctaagctcaccagagctgagactgtttttcctgatgtggactatgtaaattctactgaagctgaaaccattttggataacatcactcaaagcacccaatcatttaatgacttcactagggttgttggtggagaagatgccaaaccaggtcaattcccttggcaggttgttttgaatggtaaagttgatgcattctgtggaggctctattgttaatgaaaaatggattgtaactgctgcccactgtgttgaaactggtgttaaaattacagttgtggcaggtgaacataatattgaggagacagaacatacagagcaaaagagaaatgtgattagaattattcctcaccacaactacaatgcagctattaataagtacaaccatgacattgcccttctggaactggatgaacccttagtgctaaacagctatgttacacctatttgcattgctgacaaggaatacaccaacatcttcctcaaatttggatctggctatgtaagtggctggggaagagtcttccacaaagggagatcagctttagttcttcagtaccttagagttccacttgttgacagagccacatgtcttaggtctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatccaggtatgtcaactggattaaggaaaaaacaaagctcacttaacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgttaggtgagcttagtcttttcttttatccaattcacgtagcgagagaccttcgtatagatgccatatttccccttcatcgcacattcctccccccaacttattatcccggtcaagaaacttgttccttcgacttcagtgacgtgtggtccacctgaatcaccttggcatgagtcgcgaccgccctcgtgaaacccagcacaaaacatgttattgtaaatcgtaaatttcgtggaccgaagacaggtcgctctatcgaccaacgggacgcgcaaatattgcagaacgagggctgaccgacctttgtggaagacccgcccccacccactcacatatccgctcccaaatttcaagaagatatttgtatattctttatcggctatacaaatcggggtaacataggagttaagtacgagtggctcgtccagctccaggagggctatatcatggttgtacttgtttatagcggcattataattgtgatggggtatgatcctgataacattccttttctgttcagtatgctcagtttcttcaatgttgtgttcgccagccacgaccgtaatcttaacccccgtctcgacacagtgtgcggccgttacaatccacttttcattgactatggagcccccacaaaacgcgtcgacttttccgttgagcaccacctgccatggaaattggccaggtttagcgtcctcgcccccgacaaccctagtaaagtcattaaatgactgtgtggattgtgttatattatcaagaatcgtttcggcttcagtagagttaacgtagtccacatcgggaaaaactgtctcggcccttgtcaactttgatgtctgggacacacttacccgaccgcacgggaagggcaccgccggttcacagctcttttgattctcagcgagccggtagccctcagtgcaactacacacaactttgttgtcggcggaatttttacagaattgctcgcatcgtccatttttaatgttgcaggtgacgtccaactcgcagttttttccttcaaaaccaaaagggcaccaacactcgtaggaatttatatcgtctttacaactccccccattcagacatggattagattcgcattggtccccatcgacatattgcttccagaactcagtggtccgttctgtattctcaaacacctcgcgcgcttcttcaaaactgcatttttcctccatacactctcgctccaagttcccttgcacgaattcttcaagctttcctgagttataccttttaggccggttaagtatcttattcgcgttttcgtggtccagaaaaactgtggaaacagggagagaaaaaccacacaacatatttaaagattgatgaagacaactaactgtaatatgctgctttttgttcttctcttcactgacctaagagatct

SEQ ID NO: 213 - DNA, 3280 bp, synthetic construct
  misc_feature 1..3280: note = Synthetic
  source 1..3280: mol_type = other DNA
agatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgttaggtgagcttagtcttttcttttatccaattcacgtagcgagagaccttcgtatagatgccatatttccccttcatcgcacattcctccccccaacttattatcccggtcaagaaacttgttccttcgacttcagtgacgtgtggtccacctgaatcaccttggcatgagtcgcgaccgccctcgtgaaacccagcacaaaacatgttattgtaaatcgtaaatttcgtggaccgaagacaggtcgctctatcgaccaacgggacgcgcaaatattgcagaacgagggctgaccgacctttgtggaagacccgcccccacccactcacatatccgctcccaaatttcaagaagatatttgtatattctttatcggctatacaaatcggggtaacataggagttaagtacgagtggctcgtccagctccaggagggctatatcatggttgtacttgtttatagcggcattataattgtgatggggtatgatcctgataacattccttttctgttcagtatgctcagtttcttcaatgttgtgttcgccagccacgaccgtaatcttaacccccgtctcgacacagtgtgcggccgttacaatccacttttcattgactatggagcccccacaaaacgcgtcgacttttccgttgagcaccacctgccatggaaattggccaggtttagcgtcctcgcccccgacaaccctagtaaagtcattaaatgactgtgtggattgtgttatattatcaagaatcgtttcggcttcagtagagttaacgtagtccacatcgggaaaaactgtctcggcccttgtcaactttgatgtctgggacacacttacccgaccgcacgggaagggcaccgccggttcacagctcttttgattctcagcgagccggtagccctcagtgcaactacacacaactttgttgtcggcggaatttttacagaattgctcgcatcgtccatttttaatgttgcaggtgacgtccaactcgcagttttttccttcaaaaccaaaagggcaccaacactcgtaggaatttatatcgtctttacaactccccccattcagacatggattagattcgcattggtccccatcgacatattgcttccagaactcagtggtccgttctgtattctcaaacacctcgcgcgcttcttcaaaactgcatttttcctccatacactctcgctccaagttcccttgcacgaattcttcaagctttcctgagttataccttttaggccggttaagtatcttattcgcgttttcgtggtccagaaaaactgtggaaacagggagagaaaaaccacacaacatatttaaagattgatgaagacaactaactgtaatatgctgctttttgttcttctcttcactgacctaagagatct

SEQ ID NO: 214 - DNA, 3280 bp, synthetic construct
  misc_feature 1..3280: note = Synthetic
  source 1..3280: mol_type = other DNA
agatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcagggaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgcgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggacaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgttaggtgagcttagtcttttcttttatccaattcacgtagcgagagaccttcgtatagatgccatatttccccttcatcgcacattcctccccccaacttattatcccggtcaagaaacttgttccttcgacttcagtgacgtgtggtccacctgaatcaccttggcatgagtcgcgaccgccctcgtgaaacccagcacaaaacatgttattgtaaatcgtaaatttcgtggaccgaagacaggtcgctctatcgaccaacgggacgcgcaaatattgcagaacgagggctgatcgacctttgtggaagacccgcccccacccactcacatatccgctcccaaatttcaagaagatatttgtatattctttatcggctatacaaatcggggtaacataggagttaagtacgagtggctcgtccagctccaggagggctatatcatggttgtacttgtttatagcggcattataattgtgatggggtatgatcctgataacattccttttctgttcagtatgctcagtttcttcaatgttgtgttcgccagccacgaccgtaatcttaacccccgtctcgacacagtgtgcggccgttacaatccacttttcattgactatggagcccccacaaaacgcgtcgacttttccgttgagcaccacctgccatggaaattggccaggtttagcgtcctcgcccccgacaaccctagtaaagtcattaaatgactgtgtggattgtgttatattatcaagaatcgtttcggcttcagtagagttaacgtagtccacatcgggaaaaactgtctcggcccttgtcaactttgatgtctgggacacacttacccgaccgcacgggaagggcaccgccggttcacagctcttttgattctcagcgagccggtagccctcagtgcaactacacacaactttgttgtcggcggaatttttacagaattgctcgcatcgtccatttttaatgttgcaggtgacgtccaactcgcagttttttccttcaaaaccaaaagggcaccaacactcgtaggaatttatatcgtctttacaactccccccattcagacatggattagattcgcattggtccccatcgacatattgcttccagaactcagtggtccgttctgtattctcaaacacctcgcgcgcttcttcaaaactgcatttttcctccatacactctcgctccaagttcccttgcacgaattcttcaagctttcctgagttataccttttaggccggttaagtatcttattcgcgttttcgtggtccagaaaaactgtggaaacagggagagaaaaaccacacaacatatttaaagattgatgaagacaactaactgtaatatgctgctttttgttcttctcttcactgacctaagagatct

SEQ ID NO: 215 - DNA, 3280 bp, synthetic construct
  misc_feature 1..3280: note = Synthetic
  source 1..3280: mol_type = other DNA
agatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagtttggttttttctttgatccagttcacatatctggacaccttggtgtagatgccatacttgcccttcatggcacattcctctccccagctgatgatgcctgtcaggaagctggtgccctccacctctgtcacgtgtgggcctccagagtctccttggcagctgtctctgcctccctcatggaagccagcacagaacatgttgttgtagatggtgaacttggtgcttctcaggcaggtggctctgtccaccagggggactctcaggtattgcagcaccagggcagacctgcccttgtggaacactctgccccagccagacacgtagccagagccaaacttcaggaagatgttggtgtactctttgtctgcaatgcagatgggggtaacatagctgttcagcaccagaggctcatcaagttccagcagggcaatgtcatggttgtacttgttgatggctgcattgtagttgtggtgagggatgatcctgatcacatttctcttctgctctgtgtgctctgtttcttctatgttgtgttcaccagccaccactgtgatcttgacacctgtttccacacagtgggcagctgtcacaatccatttttcattcacaatgctgcccccacagaaggcatccaccttgccattcagcaccacctgccatgggaactggccaggcttggcatcctctcctcccaccactctggtgaagtcattgaagctctgggtgctctgggtgatgttgtccaggatggtctcagcctcagtgctgttcacatagtccacatcagggaacactgtttcagccctggtcagcttggaggtctgggacacagacactctgccacaggggaaagggacagcaggttcacagctcttctggttttcagccagcctgtagccctctgtgcagctgcacaccaccttgttgtctgctgagttcttgcagaactgttcacatctgccattcttgatgttgcaggtgacatccagctcacagttcttgccctcaaagccaaaagggcaccagcactcatagctgttgatgtcatctttgcagctgcctccattcaggcaagggttgctctcacactggtctccatcaacgtactgcttccagaactcagtggttctctctgtgttttcaaacacttctctggcttcctcaaagctgcacttctcttccatgcactccctctccaggttgccctgcacaaactcctccagcttgccactgttgtatctcttgggtctgttgaggatcttgttggcattctcatggtccaggaaaactgtggaaacagggagagaaaaaccacacaacatatttaaagattgatgaagacaactaactgtaatatgctgctttttgttcttctcttcactgacctaagagatct

SEQ ID NO: 216 - DNA, 3280 bp, synthetic construct
  misc_feature 1..3280: note = Synthetic
  source 1..3280: mol_type = other DNA
agatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagcttggttttctctttgatccagttcacatatctgctaaccttggtgtagatgccatacttgcccttcatggcacactcttccccccagctgatgatgcctgtcaggaagctggtgccctccacctctgtcacatgtgggcctccagagtctccctggcagctgtctctgcctccctcatggaagccagcacagaacatgttgttgtagatggtgaacttggtgcttctcaggcaggtggccctgtccaccagggggactctcaggtattgcagtaccagggcagacctgcccttgtggaacactctgccccagccagacacatagccactgccaaacttgaggaagatgttggtgtactccttgtcagcaatgcagatgggggtaacatagctgttcagcaccagaggctcatcaagttccagcagggcaatgtcatggttgtacttgttgatggctgcattgtagttgtggtgagggatgatcctgatcacatttctcttctgctctgtgtgctctgtttcttctatgttgtgctctccagccaccactgtgattttgacacctgtctccacacagtgggcagctgtcacaatccatttctcattcacaatgctgcctccacagaaggcatccactttgccattcagcaccacctgccatgggaactggccaggcttggcatcctctccacccaccaccctggtgaagtcattgaaggactgggtgctctgggtgatgttgtccaggatggtttcagcctctgtgctgttcacatagtccacatcagggaacacagtctcagccctggtcagcttgctggtctgggacacagacactctgccacaggggaaagggacagcaggttcacaggacttctggttttctgccagcctgtagccctctgtgcagctgcacaccaccttgttgtcagcagagttcttgcagaactgttcacatctgccattcttgatgttgcaggtgacatccagctcacagtttttgccttcaaagccaaaagggcaccagcattcatagctgttgatgtcatccttgcagctgcctccattcaggcaagggttgctctcacactggtctccatccacatactgcttccagaactcagtggttctctctgtgttttcaaacacttccctggcttcttcaaagctgcacttttcttccatgcactctctctccaggttgccttgcacaaactcctccagcttgccagagttgtatctcttgggtctgttcaggatcttgttggcattctcatggtccaggaaaactgtggaaacagggagagaaaaaccacacaacatatttaaagattgatgaagacaactaactgtaatatgctgctttttgttcttctcttcactgacctaagagatct

SEQ ID NO: 217 - DNA, 3280 bp, synthetic construct
  misc_feature 1..3280: note = Synthetic
  source 1..3280: mol_type = other DNA
agatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagcttggttttctctttgatccagttcacatatctgctaaccttggtgtagatgccatacttgcccttcatggcacactcttccccccagctgatgatgcctgtcaggaagctggtgccctccacctctgtcacgtgtgggcctccagagtctccctggcagctgtctctgcctccctcatggaagccggcacagaacatgttgttgtagatggtgaacttggtgcttctcaggcaggtggccctgtccaccagggggactctcaggtattgcagtaccagggcagacctgcccttgtggaacactctgccccagccagacacatagccactgccaaacttgaggaagatgttggtgtactccttgtcagcaatgcagatgggggtaacatagctgttcagcaccagaggctcatcaagttccagcagggcaatgtcgtggttgtacttgttgatggctgcattgtagttgtggtgagggatgatccggatcacatttctcttctgctctgtgtgctctgtttcttctatgttgtgctctccagccaccactgtgattttgacacctgtctccacacagtgggcggctgtcacaatccatttctcattcacaatgctgcctccacagaaggcatccactttgccattcagcaccacctgccatgggaactggccaggcttggcatcctctccacccaccacccgggtgaagtcattgaaggactgggtgctctgggtgatgttgtccaggatggtttcagcctctgtgctgttcacgtagtccacatcagggaacacagtctcagccctggtcagcttgctggtctgggacacagacactctgccacaggggaaagggacagcaggttcacaggacttctggttttctgccagcctgtagccctctgtgcagctgcacaccaccttgttgtcagcagagttcttgcagaactgttcacatctgccattcttgatgttgcaggtgacatccagctcacagtttttgccttcaaagccaaaagggcaccagcattcgtagctgttgatgtcatccttgcagctgcctccattcaggcaagggttgctctcacactggtctccatccacgtactgcttccagaactcagtggttctctctgtgttttcaaacacttcccgggcttcttcaaagctgcacttttcttccatgcactctctctccaggttgccttgcacaaactcctccagcttgccagagttgtatctcttgggtctgttcaggatcttgttggcattctcatggtccaggaaaactgtggaaacagggagagaaaaaccacacaacatatttaaagattgatgaagacaactaactgtaatatgctgctttttgttcttctcttcactgacctaagagatct

SEQ ID NO: 218 - DNA, 3280 bp, synthetic construct
  misc_feature 1..3280: note = Synthetic
  source 1..3280: mol_type = other DNA
agatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagcttggttttctctttgatccagttgacatatctgctcaccttggtgtagatgccatacttgcccttcatggcacactcttccccccagctgatgatgcctgtcaggaagctggtgccctccacctctgtcacatgtgggcctccagagtctccctggcagctgtctctgcctccctcatggaagccagcacagaacatgttgttgtagatggtgaacttggtgcttctcaggcaggtggctctgtccaccagggggaccctcaggtattgcagcaccagggcagacctgcccttgtggaacactctgccccagccagacacatagccagagccaaacttcaggaagatgttggtgtactccttgtcagcaatgcagatgggggtaacatagctgttcagcaccagaggctcatcaagttccagcagggcaatgtcatggttgtacttgttgatggcagcattgtagttgtggtgagggatgattctgatcacattccttttctgctctgtgtgctctgtttcctcaatgttgtgctctccagccaccactgtgatcttgacacctgtctccacacagtgggcagctgtcacaatccacttttcattcacaatgctgcctccacagaaggcatccactttgccattgagcaccacctgccatgggaactggccaggcttggcatcctctccacccaccaccctggtgaagtcattgaaggactgggtgctctgggtgatgttgtccaggatggtttcagcttctgtgctgttcacatagtccacatcagggaacactgtctcagccctggtcagtttgctggtctggctcactgacactctgccacaggggaaaggcacagcaggttcacaggacttctggttttctgccagcctgtagccctctgtgcagctgcacaccaccttgttgtcagcagagttcttgcagaactgttcacatctgccatttttgatgttgcaggtgacatccagctcacagttcttgccttcaaagccaaaagggcaccagcattcatagctgttgatgtcatccttgcaggagcctccattcaggcaagggttgctctcacactggtctccatcaacatactgcttccagaactcagtggttctttctgtgttctcaaacacttccctggcttcctcaaagctgcacttctcttccatgcactccctctccaggttgccttgcacaaattcctccagcttgccactgttgtatctcttgggtctgttcaggatcttgttggcattctcatggtccaggaaaactgtggaaacagggagagaaaaaccacacaacatatttaaagattgatgaagacaactaactgtaatatgctgctttttgttcttctcttcactgacctaagagatct

SEQ ID NO: 219 - DNA, 3280 bp, synthetic construct
  misc_feature 1..3280: note = Synthetic
  source 1..3280: mol_type = other DNA
agatctcttaggtcagtgaagagaagaacaaaaagcagcatattacagttagttgtcttcatcaatctttaaatatgttgtgtggtttttctctccctgtttccacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcacttaacctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagcttggttttctctttgatccagttgacatatctgctcaccttggtgtagatgccgtacttgcccttcatggcacactcttccccccagctgatgatgcctgtcaggaagctggtgccctccacctctgtcacgtgtgggcctccagagtctccctggcagctgtctctgcctccctcatggaagccggcacagaacatgttgttgtagatggtgaacttggtgcttctcaggcaggtggctctgtccaccagggggacccgcaggtattgcagcaccagggcagacctgcccttgtggaacactctgccccagccagacacgtagccagagccaaacttcaggaagatgttggtgtactccttgtcggcaatgcagatgggggtaacatagctgttcagcaccagaggctcatcaagttccagcagggcaatgtcgtggttgtacttgttgatggcagcattgtagttgtggtgagggatgattctgatcacattccgtttctgctctgtgtgctctgtttcctcgatgttgtgctctccagccaccactgtgatcttgacacctgtctccacacagtgggcggctgtcacgatccacttttcattcacaatgctgcctccacagaaggcatccactttgccattgagcaccacctgccatgggaactggccaggcttggcatcctctccacccaccacccgggtgaagtcattgaaggactgggtgctctgggtgatgttgtccaggatggtttcagcttctgtgctgttcacgtagtccacatcagggaacactgtctcagcccgggtcagtttgctggtctggctcactgacactctgccacaggggaaaggcacggcaggttcacaggacttctggttttctgccagcctgtagccctctgtgcagctgcacaccaccttgttgtcagcagagttcttgcagaactgttcacatctgccatttttgatgttgcaggtgacatccagctcacagttcttgccttcaaagccaaaagggcaccagcattcgtagctgttgatgtcatccttgcaggagcctccattcaggcaagggttgctctcgcactggtctccatcaacgtactgcttccagaactcagtggttctttctgtgttctcgaacacttcccgggcttcctcaaagctgcacttctcttccatgcactccctctccaggttgccttgcacaaattcctccagcttgccactgttgtatctcttgggtctgttcaggatcttgttggcattctcatggtccaggaaaactgtggaaacagggagagaaaaaccacacaacatatttaaagattgatgaagacaactaactgtaatatgctgctttttgttcttctcttcactgacctaagagatct

SEQ ID NO: 220 - DNA, 3280 bp, synthetic construct
  misc_feature 1..3280: note = Synthetic
  source 1..3280: mol_type = other DNA
agatctcttaggtcagtgaagagaagaacaaaaagcagcatattacagttagttgtcttcatcaatctttaaatatgttgtgtggtttttctctccctgtttccacagtttttcttgatcatgaaaatgccaacaaaattctgaataggccaaagaggtataattcagggaaattggaagagtttgttcaagggaaccttgagagagaatgtatggaagaaaagtgtagttttgaagaagcaagagaagtttttgaaaacactgaaagaacaactgaattttggaagcagtatgttgatggagatcagtgcgagtccaatccatgtttaaatgggggcagttgcaaggatgacattaattcctatgaatgttggtgtccctttggatttgaaggaaagaactgtgaattagatgtaacatgtaacattaagaatggcagatgtgagcagttttgtaaaaatagtgctgataacaaggtggtttgctcctgtactgagggatatagacttgcagaaaaccagaagtcctgtgaaccagcagtgccatttccatgtggaagagtttctgtttcacaaacttctaagctcaccagagctgagactgtttttcctgatgtggactatgtaaattctactgaagctgaaaccattttggataacatcactcaaagcacccaatcatttaatgacttcactagggttgttggtggagaagatgccaaaccaggacaattcccttggcaggttgttttgaatggtaaagttgatgcattctgtggaggctctattgttaatgaaaaatggattgtaactgctgcccactgtgttgaaactggtgttaaaattacagttgtggcaggtgaacataatattgaggagacagaacatacagagcaaaagagaaatgtgattagaattattcctcaccacaactacaatgcagctattaataagtacaaccatgacattgcccttctggaactggatgaacccttagtgctaaacagctatgttacacctatttgcattgctgacaaggaatacaccaacatcttcctcaaatttggatctggctatgtaagtggctggggaagagtcttccacaaagggagatcagctttagttcttcagtaccttagagttccacttgttgacagagccacatgtcttaggtctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatccaggtatgtcaactggattaaggaaaaaacaaagctcacttaacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagtttggttttttctttgatccagttcacatatctggacaccttggtgtagatgccatacttgcccttcatggcacattcctctccccagctgatgatgcctgtcaggaagctggtgccctccacctctgtcacgtgtgggcctccagagtctccttggcagctgtctctgcctccctcatggaagccagcacagaacatgttgttgtagatggtgaacttggtgcttctcaggcaggtggctctgtccaccagggggactctcaggtattgcagcaccagggcagacctgcccttgtggaacactctgccccagccagacacgtagccagagccaaacttcaggaagatgttggtgtactctttgtctgcaatgcagatgggggtaacatagctgttcagcaccagaggctcatcaagttccagcagggcaatgtcatggttgtacttgttgatggctgcattgtagttgtggtgagggatgatcctgatcacatttctcttctgctctgtgtgctctgtttcttctatgttgtgttcaccagccaccactgtgatcttgacacctgtttccacacagtgggcagctgtcacaatccatttttcattcacaatgctgcccccacagaaggcatccaccttgccattcagcaccacctgccatgggaactggccaggcttggcatcctctcctcccaccactctggtgaagtcattgaagctctgggtgctctgggtgatgttgtccaggatggtctcagcctcagtgctgttcacatagtccacatcagggaacactgtttcagccctggtcagcttggaggtctgggacacagacactctgccacaggggaaagggacagcaggttcacagctcttctggttttcagccagcctgtagccctctgtgcagctgcacaccaccttgttgtctgctgagttcttgcagaactgttcacatctgccattcttgatgttgcaggtgacatccagctcacagttcttgccctcaaagccaaaagggcaccagcactcatagctgttgatgtcatctttgcagctgcctccattcaggcaagggttgctctcacactggtctccatcaacgtactgcttccagaactcagtggttctctctgtgttttcaaacacttctctggcttcctcaaagctgcacttctcttccatgcactccctctccaggttgccctgcacaaactcctccagcttgccactgttgtatctcttgggtctgttgaggatcttgttggcattctcatggtccaggaaaactgtggaaacagggagagaaaaaccacacaacatatttaaagattgatgaagacaactaactgtaatatgctgctttttgttcttctcttcactgacctaagagatct

SEQ ID NO: 221 - DNA, 3280 bp, synthetic construct
  misc_feature 1..3280: note = Synthetic
  source 1..3280: mol_type = other DNA
agatctcttaggtcagtgaagagaagaacaaaaagcagcatattacagttagttgtcttcatcaatctttaaatatgttgtgtggtttttctctccctgtttccacagtttttcttgatcatgaaaatgccaacaaaattctgaataggccaaagaggtataattcagggaaattggaagagtttgttcaagggaaccttgagagagaatgtatggaagaaaagtgtagttttgaagaagcaagagaagtttttgaaaacactgaaagaacaactgaattttggaagcagtatgttgatggagatcagtgcgagtccaatccatgtttaaatgggggcagttgcaaggatgacattaattcctatgaatgttggtgtccctttggatttgaaggaaagaactgtgaattagatgtaacatgtaacattaagaatggcagatgtgagcagttttgtaaaaatagtgctgataacaaggtggtttgctcctgtactgagggatatagacttgcagaaaaccagaagtcctgtgaaccagcagtgccatttccatgtggaagagtttctgtttcacaaacttctaagctcaccagagctgagactgtttttcctgatgtggactatgtaaattctactgaagctgaaaccattttggataacatcactcaaagcacccaatcatttaatgacttcactagggttgttggtggagaagatgccaaaccaggacaattcccttggcaggttgttttgaatggtaaagttgatgcattctgtggaggctctattgttaatgaaaaatggattgtaactgctgcccactgtgttgaaactggtgttaaaattacagttgtggcaggtgaacataatattgaggagacagaacatacagagcaaaagagaaatgtgattagaattattcctcaccacaactacaatgcagctattaataagtacaaccatgacattgcccttctggaactggatgaacccttagtgctaaacagctatgttacacctatttgcattgctgacaaggaatacaccaacatcttcctcaaatttggatctggctatgtaagtggctggggaagagtcttccacaaagggagatcagctttagttcttcagtaccttagagttccacttgttgacagagccacatgtcttaggtctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatccaggtatgtcaactggattaaggaaaaaacaaagctcacttaacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagcttggttttctctttgatccagttcacatatctgctaaccttggtgtagatgccatacttgcccttcatggcacactcttccccccagctgatgatgcctgtcaggaagctggtgccctccacctctgtcacatgtgggcctccagagtctccctggcagctgtctctgcctccctcatggaagccagcacagaacatgttgttgtagatggtgaacttggtgcttctcaggcaggtggccctgtccaccagggggactctcaggtattgcagtaccagggcagacctgcccttgtggaacactctgccccagccagacacatagccactgccaaacttgaggaagatgttggtgtactccttgtcagcaatgcagatgggggtaacatagctgttcagcaccagaggctcatcaagttccagcagggcaatgtcatggttgtacttgttgatggctgcattgtagttgtggtgagggatgatcctgatcacatttctcttctgctctgtgtgctctgtttcttctatgttgtgctctccagccaccactgtgattttgacacctgtctccacacagtgggcagctgtcacaatccatttctcattcacaatgctgcctccacagaaggcatccactttgccattcagcaccacctgccatgggaactggccaggcttggcatcctctccacccaccaccctggtgaagtcattgaaggactgggtgctctgggtgatgttgtccaggatggtttcagcctctgtgctgttcacatagtccacatcagggaacacagtctcagccctggtcagcttgctggtctgggacacagacactctgccacaggggaaagggacagcaggttcacaggacttctggttttctgccagcctgtagccctctgtgcagctgcacaccaccttgttgtcagcagagttcttgcagaactgttcacatctgccattcttgatgttgcaggtgacatccagctcacagtttttgccttcaaagccaaaagggcaccagcattcatagctgttgatgtcatccttgcagctgcctccattcaggcaagggttgctctcacactggtctccatccacatactgcttccagaactcagtggttctctctgtgttttcaaacacttccctggcttcttcaaagctgcacttttcttccatgcactctctctccaggttgccttgcacaaactcctccagcttgccagagttgtatctcttgggtctgttcaggatcttgttggcattctcatggtccaggaaaactgtggaaacagggagagaaaaaccacacaacatatttaaagattgatgaagacaactaactgtaatatgctgctttttgttcttctcttcactgacctaagagatct

SEQ ID NO: 222 - DNA, 3280 bp, synthetic construct
  misc_feature 1..3280: note = Synthetic
  source 1..3280: mol_type = other DNA
agatctcttaggtcagtgaagagaagaacaaaaagcagcatattacagttagttgtcttcatcaatctttaaatatgttgtgtggtttttctctccctgtttccacagtttttcttgatcatgaaaatgccaacaaaattctgaataggccaaagaggtataattcagggaaattggaagagtttgttcaagggaaccttgagagagaatgtatggaagaaaagtgtagttttgaagaagcaagagaagtttttgaaaacactgaaagaacaactgaattttggaagcagtatgttgatggagatcagtgcgagtccaatccatgtttaaatgggggcagttgcaaggatgacattaattcctatgaatgttggtgtccctttggatttgaaggaaagaactgtgaattagatgtaacatgtaacattaagaatggcagatgtgagcagttttgtaaaaatagtgctgataacaaggtggtttgctcctgtactgagggatatagacttgcagaaaaccagaagtcctgtgaaccagcagtgccatttccatgtggaagagtttctgtttcacaaacttctaagctcaccagagctgagactgtttttcctgatgtggactatgtaaattctactgaagctgaaaccattttggataacatcactcaaagcacccaatcatttaatgacttcactagggttgttggtggagaagatgccaaaccaggacaattcccttggcaggttgttttgaatggtaaagttgatgcattctgtggaggctctattgttaatgaaaaatggattgtaactgctgcccactgtgttgaaactggtgttaaaattacagttgtggcaggtgaacataatattgaggagacagaacatacagagcaaaagagaaatgtgattagaattattcctcaccacaactacaatgcagctattaataagtacaaccatgacattgcccttctggaactggatgaacccttagtgctaaacagctatgttacacctatttgcattgctgacaaggaatacaccaacatcttcctcaaatttggatctggctatgtaagtggctggggaagagtcttccacaaagggagatcagctttagttcttcagtaccttagagttccacttgttgacagagccacatgtcttaggtctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatccaggtatgtcaactggattaaggaaaaaacaaagctcacttaacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagcttggttttctctttgatccagttcacatatctgctaaccttggtgtagatgccatacttgcccttcatggcacactcttccccccagctgatgatgcctgtcaggaagctggtgccctccacctctgtcacgtgtgggcctccagagtctccctggcagctgtctctgcctccctcatggaagccggcacagaacatgttgttgtagatggtgaacttggtgcttctcaggcaggtggccctgtccaccagggggactctcaggtattgcagtaccagggcagacctgcccttgtggaacactctgccccagccagacacatagccactgccaaacttgaggaagatgttggtgtactccttgtcagcaatgcagatgggggtaacatagctgttcagcaccagaggctcatcaagttccagcagggcaatgtcgtggttgtacttgttgatggctgcattgtagttgtggtgagggatgatccggatcacatttctcttctgctctgtgtgctctgtttcttctatgttgtgctctccagccaccactgtgattttgacacctgtctccacacagtgggcggctgtcacaatccatttctcattcacaatgctgcctccacagaaggcatccactttgccattcagcaccacctgccatgggaactggccaggcttggcatcctctccacccaccacccgggtgaagtcattgaaggactgggtgctctgggtgatgttgtccaggatggtttcagcctctgtgctgttcacgtagtccacatcagggaacacagtctcagccctggtcagcttgctggtctgggacacagacactctgccacaggggaaagggacagcaggttcacaggacttctggttttctgccagcctgtagccctctgtgcagctgcacaccaccttgttgtcagcagagttcttgcagaactgttcacatctgccattcttgatgttgcaggtgacatccagctcacagtttttgccttcaaagccaaaagggcaccagcattcgtagctgttgatgtcatccttgcagctgcctccattcaggcaagggttgctctcacactggtctccatccacgtactgcttccagaactcagtggttctctctgtgttttcaaacacttcccgggcttcttcaaagctgcacttttcttccatgcactctctctccaggttgccttgcacaaactcctccagcttgccagagttgtatctcttgggtctgttcaggatcttgttggcattctcatggtccaggaaaactgtggaaacagggagagaaaaaccacacaacatatttaaagattgatgaagacaactaactgtaatatgctgctttttgttcttctcttcactgacctaagagatct

SEQ ID NO: 223 - DNA, 3280 bp, synthetic construct
  misc_feature 1..3280: note = Synthetic
  source 1..3280: mol_type = other DNA
agatctcttaggtcagtgaagagaagaacaaaaagcagcatattacagttagttgtcttcatcaatctttaaatatgttgtgtggtttttctctccctgtttccacagtttttcttgatcatgaaaatgccaacaaaattctgaataggccaaagaggtataattcagggaaattggaagagtttgttcaagggaaccttgagagagaatgtatggaagaaaagtgtagttttgaagaagcaagagaagtttttgaaaacactgaaagaacaactgaattttggaagcagtatgttgatggagatcagtgcgagtccaatccatgtttaaatgggggcagttgcaaggatgacattaattcctatgaatgttggtgtccctttggatttgaaggaaagaactgtgaattagatgtaacatgtaacattaagaatggcagatgtgagcagttttgtaaaaatagtgctgataacaaggtggtttgctcctgtactgagggatatagacttgcagaaaaccagaagtcctgtgaaccagcagtgccatttccatgtggaagagtttctgtttcacaaacttctaagctcaccagagctgagactgtttttcctgatgtggactatgtaaattctactgaagctgaaaccattttggataacatcactcaaagcacccaatcatttaatgacttcactagggttgttggtggagaagatgccaaaccaggacaattcccttggcaggttgttttgaatggtaaagttgatgcattctgtggaggctctattgttaatgaaaaatggattgtaactgctgcccactgtgttgaaactggtgttaaaattacagttgtggcaggtgaacataatattgaggagacagaacatacagagcaaaagagaaatgtgattagaattattcctcaccacaactacaatgcagctattaataagtacaaccatgacattgcccttctggaactggatgaacccttagtgctaaacagctatgttacacctatttgcattgctgacaaggaatacaccaacatcttcctcaaatttggatctggctatgtaagtggctggggaagagtcttccacaaagggagatcagctttagttcttcagtaccttagagttccacttgttgacagagccacatgtcttaggtctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatccaggtatgtcaactggattaaggaaaaaacaaagctcacttaacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagcttggttttctctttgatccagttgacatatctgctcaccttggtgtagatgccatacttgcccttcatggcacactcttccccccagctgatgatgcctgtcaggaagctggtgccctccacctctgtcacatgtgggcctccagagtctccctggcagctgtctctgcctccctcatggaagccagcacagaacatgttgttgtagatggtgaacttggtgcttctcaggcaggtggctctgtccaccagggggaccctcaggtattgcagcaccagggcagacctgcccttgtggaacactctgccccagccagacacatagccagagccaaacttcaggaagatgttggtgtactccttgtcagcaatgcagatgggggtaacatagctgttcagcaccagaggctcatcaagttccagcagggcaatgtcatggttgtacttgttgatggcagcattgtagttgtggtgagggatgattctgatcacattccttttctgctctgtgtgctctgtttcctcaatgttgtgctctccagccaccactgtgatcttgacacctgtctccacacagtgggcagctgtcacaatccacttttcattcacaatgctgcctccacagaaggcatccactttgccattgagcaccacctgccatgggaactggccaggcttggcatcctctccacccaccaccctggtgaagtcattgaaggactgggtgctctgggtgatgttgtccaggatggtttcagcttctgtgctgttcacatagtccacatcagggaacactgtctcagccctggtcagtttgctggtctggctcactgacactctgccacaggggaaaggcacagcaggttcacaggacttctggttttctgccagcctgtagccctctgtgcagctgcacaccaccttgttgtcagcagagttcttgcagaactgttcacatctgccatttttgatgttgcaggtgacatccagctcacagttcttgccttcaaagccaaaagggcaccagcattcatagctgttgatgtcatccttgcaggagcctccattcaggcaagggttgctctcacactggtctccatcaacatactgcttccagaactcagtggttctttctgtgttctcaaacacttccctggcttcctcaaagctgcacttctcttccatgcactccctctccaggttgccttgcacaaattcctccagcttgccactgttgtatctcttgggtctgttcaggatcttgttggcattctcatggtccaggaaaactgtggaaacagggagagaaaaaccacacaacatatttaaagattgatgaagacaactaactgtaatatgctgctttttgttcttctcttcactgacctaagagatct

SEQ ID NO: 224 - DNA, 3280 bp, synthetic construct
  misc_feature 1..3280: note = Synthetic
  source 1..3280: mol_type = other DNA
agatctcttaggtcagtgaagagaagaacaaaaagcagcatattacagttagttgtcttcatcaatctttaaatatgttgtgtggtttttctctccctgtttccacagtttttcttgatcatgaaaatgccaacaaaattctgaataggccaaagaggtataattcagggaaattggaagagtttgttcaagggaaccttgagagagaatgtatggaagaaaagtgtagttttgaagaagcaagagaagtttttgaaaacactgaaagaacaactgaattttggaagcagtatgttgatggagatcagtgcgagtccaatccatgtttaaatgggggcagttgcaaggatgacattaattcctatgaatgttggtgtccctttggatttgaaggaaagaactgtgaattagatgtaacatgtaacattaagaatggcagatgtgagcagttttgtaaaaatagtgctgataacaaggtggtttgctcctgtactgagggatatagacttgcagaaaaccagaagtcctgtgaaccagcagtgccatttccatgtggaagagtttctgtttcacaaacttctaagctcaccagagctgagactgtttttcctgatgtggactatgtaaattctactgaagctgaaaccattttggataacatcactcaaagcacccaatcatttaatgacttcactagggttgttggtggagaagatgccaaaccaggacaattcccttggcaggttgttttgaatggtaaagttgatgcattctgtggaggctctattgttaatgaaaaatggattgtaactgctgcccactgtgttgaaactggtgttaaaattacagttgtggcaggtgaacataatattgaggagacagaacatacagagcaaaagagaaatgtgattagaattattcctcaccacaactacaatgcagctattaataagtacaaccatgacattgcccttctggaactggatgaacccttagtgctaaacagctatgttacacctatttgcattgctgacaaggaatacaccaacatcttcctcaaatttggatctggctatgtaagtggctggggaagagtcttccacaaagggagatcagctttagttcttcagtaccttagagttccacttgttgacagagccacatgtcttaggtctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatccaggtatgtcaactggattaaggaaaaaacaaagctcacttaacctctactgtgccttctagttgccagccatctgttgtttgcccctcccccttgccttccttgaccctggaaggtgccactcccactgtcctttcctaataaaatgaggaaattgcatcacattgtctgagtaggtgtcattctattctggggggtggggtggggcaggacagcaagggggaggattgggaagacaatagcaggcatgctggggatgcagtgggctctatggcttctgaggtggaaagaaccagctggggctctagggggtatccccaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcaggtcagcttggttttctctttgatccagttgacatatctgctcaccttggtgtagatgccgtacttgcccttcatggcacactcttccccccagctgatgatgcctgtcaggaagctggtgccctccacctctgtcacgtgtgggcctccagagtctccctggcagctgtctctgcctccctcatggaagccggcacagaacatgttgttgtagatggtgaacttggtgcttctcaggcaggtggctctgtccaccagggggacccgcaggtattgcagcaccagggcagacctgcccttgtggaacactctgccccagccagacacgtagccagagccaaacttcaggaagatgttggtgtactccttgtcggcaatgcagatgggggtaacatagctgttcagcaccagaggctcatcaagttccagcagggcaatgtcgtggttgtacttgttgatggcagcattgtagttgtggtgagggatgattctgatcacattccgtttctgctctgtgtgctctgtttcctcgatgttgtgctctccagccaccactgtgatcttgacacctgtctccacacagtgggcggctgtcacgatccacttttcattcacaatgctgcctccacagaaggcatccactttgccattgagcaccacctgccatgggaactggccaggcttggcatcctctccacccaccacccgggtgaagtcattgaaggactgggtgctctgggtgatgttgtccaggatggtttcagcttctgtgctgttcacgtagtccacatcagggaacactgtctcagcccgggtcagtttgctggtctggctcactgacactctgccacaggggaaaggcacggcaggttcacaggacttctggttttctgccagcctgtagccctctgtgcagctgcacaccaccttgttgtcagcagagttcttgcagaactgttcacatctgccatttttgatgttgcaggtgacatccagctcacagttcttgccttcaaagccaaaagggcaccagcattcgtagctgttgatgtcatccttgcaggagcctccattcaggcaagggttgctctcgcactggtctccatcaacgtactgcttccagaactcagtggttctttctgtgttctcgaacacttcccgggcttcctcaaagctgcacttctcttccatgcactccctctccaggttgccttgcacaaattcctccagcttgccactgttgtatctcttgggtctgttcaggatcttgttggcattctcatggtccaggaaaactgtggaaacagggagagaaaaaccacacaacatatttaaagattgatgaagacaactaactgtaatatgctgctttttgttcttctcttcactgacctaagagatct

SEQ ID NO: 225 - RNA, 4423 nt, synthetic construct
  misc_feature 1..4423: note = Synthetic
  source 1..4423: mol_type = other RNA
gggaagctcagaataaacgctcaactttggccggatctgccaccatggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtgactagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 226 - RNA, 4140 nt, synthetic construct
  misc_feature 1..4140: note = Synthetic
  source 1..4140: mol_type = other RNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 227 - DNA, 4140 bp, synthetic construct
  misc_feature 1..4140: note = Synthetic
  source 1..4140: mol_type = other DNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtga

SEQ ID NO: 228 - RNA, 20 nt, synthetic construct
  misc_feature 1..20: note = Synthetic
  source 1..20: mol_type = other RNA
cactcttgtctgtggaaaca

SEQ ID NO: 229 - DNA, 20 bp, synthetic construct
  misc_feature 1..20: note = Synthetic
  source 1..20: mol_type = other DNA
cactcttgtctgtggaaaca

SEQ ID NO: 230 - RNA, 100 nt, synthetic construct
  misc_feature 1..100: note = Synthetic
  source 1..100: mol_type = other RNA
cactcttgtctgtggaaacagttttagagctagaaatagcaagttaaaataaggctagtccgttatcaacttgaaaaagtggcaccgagtcggtgctttt

SEQ ID NO: 231 - RNA, 100 nt, synthetic construct; identical to SEQ ID NO: 230
  misc_feature 1..100: note = Synthetic
  source 1..100: mol_type = other RNA

(19) United States
(12) Patent Application Publication
(10) Pub. No.: US 2023/0149563 A1
(43) Pub. Date: May 18, 2023

(54) COMPOSITIONS AND METHODS FOR EXPRESSING FACTOR IX FOR HEMOPHILIA B THERAPY

(71) Applicant: Regeneron Pharmaceuticals, Inc., Tarrytown, NY (US)

(72) Inventors: Leah Sabin, Goldens Bridge, NY (US); Christos Kyratsous, Irvington, NY (US); Evangelos Pefanis, Somers, NY (US); Sven Moller-Tank, White Plains, NY (US); Joseph Katakowski, River Vale, NJ (US); Andrew Baik, Scarsdale, NY (US); Katherine Cygnar, New York, NY (US); Poulami Samai, New York, NY (US); Philip Calafati, Tarrytown, NY (US)

(21) Appl. No.: 18/050,161

(22) Filed: Oct. 27, 2022

Related U.S. Application Data
(60) Provisional application No. 63/369,864, filed on Jul. 29, 2022.
(60) Provisional application No. 63/306,037, filed on Feb. 2, 2022.
(60) Provisional application No. 63/272,324, filed on Oct. 27, 2021.

(51) Int. Cl. A61K 48/00; C12N 15/11; C12N 9/22; C12N 15/86; C12N 15/90; C12N 5/071; A61K 38/36; A61K 38/46; A61P 7/04

(52) U.S. Cl. CPC A61K 48/0016; C12N 15/11; C12N 9/22; C12N 15/86; C12N 15/907; C12N 5/067; A61K 38/36; A61K 38/465; A61P 7/04; C12N 2750/14143; C12N 2310/20; C12N 2800/80

(57) ABSTRACT

Nucleic acid constructs and compositions that allow insertion of a FIX coding sequence into a target genomic locus such as an endogenous ALB locus and/or expression of the FIX coding sequence are provided. The nucleic acid constructs and compositions can be used in methods of introducing a F9 nucleic acid into a cell, methods of integration of a F9 nucleic acid into a target genomic locus, methods of expression of FIX in a cell, and in methods of treating hemophilia B or FIX deficiency in a subject.

### CROSS-REFERENCE TO RELATED APPLICATIONS

This application claims the benefit of U.S. Application No. 63/272,324, filed Oct. 27, 2021, U.S. Application No. 63/306,037, filed Feb. 2, 2022, and U.S. Application No. 63/369,864, filed Jul. 29, 2022, each of which is herein incorporated by reference in its entirety for all purposes.

### REFERENCE TO A SEQUENCE LISTING SUBMITTED AS A TEXT FILE VIA EFS WEB

The Sequence Listing written in file 057766-586406.xml is 516 kilobytes, was created on Oct. 27, 2022, and is hereby incorporated by reference.

### BACKGROUND

Hemophilia B is a rare congenital genetic disorder characterized by a deficiency or absence of coagulation factor IX (FIX), resulting in bleeding diathesis. It is caused by an inherited or spontaneous X-linked recessive mutation of the factor 9 (F9) gene leading to a missing or defective FIX protein. The global incidence of hemophilia B is approximately 1 in 25,000 live male births. In the U.S., the latest worldwide hemophilia survey dated 2019 reported 4,093 patients living with hemophilia B, for a worldwide total of 31,997 hemophilia B patients. World Federation of Hemophilia, “Report on the Annual Global Survey 2019” World Federation of Hemophilia (2020).

Caused by an X-linked, recessive mutation, hemophilia B mostly affects men, although female carriers may also be affected. The bleeding tendency of hemophilia B is related to the measured concentration of the factor and is classified as mild, moderate, or severe. In severe and moderately severe cases of hemophilia B, frequent, spontaneous bleeding episodes are the most common symptoms. Such bleeding episodes may occur in the muscles and joints, causing pain and movement restriction and, if left untreated, can result in long-term damage of the joint with synovitis, arthropathy and muscle weakness.

The current standard of care in the U.S. is prophylaxis and treatment of the bleeding episodes with clotting factor concentrates (CFC), either recombinant or derived from plasma of blood donors. However, allergic reactions may occur in 3-4% of CFC infusions, and around 5% of patients develop FIX neutralizing antibodies after recurring administration. Srivastava et al. “WFH Guidelines for the Management of Hemophilia,” Haemophilia 26.6:1-158 (2020). Factor replacement is also burdensome (requiring frequent intravenous administration), costly, and reduces but does not eliminate bleeding episodes, which still cause progression of joint disease and disability.

### SUMMARY

Provided are compositions comprising a nucleic acid construct comprising a first factor IX protein coding sequence or a reverse complement of the first factor IX protein coding sequence, cells comprising the compositions, and methods of using such nucleic acid constructs, such as methods of introducing a factor 9 nucleic acid into a cell, methods of integrating a factor 9 nucleic acid construct into a target gene in a cell, methods of expressing factor IX in a cell, methods of treating a factor IX deficiency in a subject, methods of treating hemophilia B in a subject, and methods of preventing or inhibiting spontaneous bleeding in a subject having hemophilia B.

In one aspect, provided are compositions comprising a nucleic acid construct comprising a first factor IX protein coding sequence or a reverse complement of the first factor IX protein coding sequence. In some such compositions, the first factor IX protein coding sequence: (I) is at least 95% identical to any one of SEQ ID NOS: 166, 165, 164, and 167-171, optionally wherein the factor IX protein coding sequence is CpG-depleted and is codon-optimized, and optionally wherein the factor IX protein is not a hyperactive factor IX variant; or (II) is at least 99% identical to any one of SEQ ID NOS: 159, 160, and 161 and is: (i) CpG-depleted; (ii) modified to mutate one or more cryptic splice donor sequences; or (iii) CpG-depleted and modified to mutate one or more cryptic splice donor sequences, and optionally wherein the factor IX protein is not a hyperactive factor IX variant.

In some such compositions, the first factor IX protein coding sequence: (I) is at least 99% identical to SEQ ID NO: 166, is CpG-depleted, and is codon-optimized, and optionally wherein the factor IX protein is not a hyperactive factor IX variant; (II) is at least 99% identical to SEQ ID NO: 165, is CpG-depleted, and is codon-optimized, and optionally wherein the factor IX protein is not a hyperactive factor IX variant; or (III) is at least 99% identical to SEQ ID NO: 159, is CpG-depleted, and is modified to mutate one or more cryptic splice donor sequences, and optionally wherein the factor IX protein is not a hyperactive factor IX variant.

In some such compositions, the first factor IX protein coding sequence: (I) is at least 99% identical to SEQ ID NO: 166, is CpG-depleted, and is codon-optimized, and optionally wherein the factor IX protein is not a hyperactive factor IX variant; or (II) is at least 99% identical to SEQ ID NO: 159, is CpG-depleted, and is modified to mutate one or more cryptic splice donor sequences, and optionally wherein the factor IX protein is not a hyperactive factor IX variant.

In some such compositions, the first factor IX protein coding sequence is at least 99% identical to SEQ ID NO: 166, is CpG-depleted, and is codon-optimized, and optionally wherein the factor IX protein is not a hyperactive factor IX variant. In some such compositions, the first factor IX protein coding sequence is at least 99% identical to SEQ ID NO: 165, is CpG-depleted, and is codon-optimized, and optionally wherein the factor IX protein is not a hyperactive factor IX variant. In some such compositions, the first factor IX protein coding sequence is at least 99% identical to SEQ ID NO: 159, is CpG-depleted, and is modified to mutate one or more cryptic splice donor sequences, and optionally wherein the factor IX protein is not a hyperactive factor IX variant. In some such compositions, the first factor IX protein coding sequence has all but one CpG dinucleotides removed or is fully CpG depleted. In some such compositions, the first factor IX protein coding sequence encodes a factor IX protein at least 99% identical to SEQ ID NO: 195, and optionally wherein the factor IX protein is not a hyperactive factor IX variant. In some such compositions, the first factor IX protein coding sequence encodes a factor IX protein comprising SEQ ID NO: 195. In some such compositions, the first factor IX protein coding sequence comprises SEQ ID NO: 166, 165, or 159. In some such compositions, the first factor IX protein coding sequence consists of SEQ ID NO: 166, 165, or 159. In some such compositions, the first factor IX protein coding sequence comprises SEQ ID NO: 166 or 159. In some such compositions, the first factor IX protein coding sequence consists of SEQ ID NO: 166 or 159. In some such compositions, the first factor IX protein coding sequence comprises SEQ ID NO: 166. In some such compositions, the first factor IX protein coding sequence consists of SEQ ID NO: 166. In some such compositions, the first factor IX protein coding sequence comprises SEQ ID NO: 165. In some such compositions, the first factor IX protein coding sequence consists of SEQ ID NO: 165. In some such compositions, the first factor IX protein coding sequence comprises SEQ ID NO: 159. In some such compositions, the first factor IX protein coding sequence consists of SEQ ID NO: 159.

In some such compositions, the nucleic acid construct comprises a splice acceptor upstream of the first factor IX protein coding sequence. In some such compositions, the nucleic acid construct comprises a polyadenylation signal downstream of the first factor IX protein coding sequence. In some such compositions, the nucleic acid construct comprises a splice acceptor upstream of the first factor IX protein coding sequence, and the nucleic acid construct comprises a polyadenylation signal downstream of the first factor IX protein coding sequence. In some such compositions, the nucleic acid construct does not comprise homology arms. In some such compositions, the nucleic acid construct comprises homology arms. In some such compositions, the nucleic acid construct does not comprise a promoter that drives the expression of the factor IX protein.

In some such compositions, the nucleic acid construct is a bidirectional construct. In some such compositions, the nucleic acid construct comprises the first factor IX protein coding sequence and a reverse complement of a second factor IX protein coding sequence, or wherein the nucleic acid construct comprises the second factor IX protein coding sequence and the reverse complement of the first factor IX protein coding sequence. In some such compositions, the first factor IX protein coding sequence and the second factor IX protein coding sequence are different but encode the same factor IX protein sequence, and optionally wherein the factor IX protein is not a hyperactive factor IX variant.

In some such compositions: (I) the first factor IX protein coding sequence is at least 99% identical to SEQ ID NO: 159 and is: (i) CpG-depleted; (ii) modified to mutate one or more cryptic splice donor sequences; or (iii) CpG-depleted and modified to mutate one or more cryptic splice donor sequences, and optionally wherein the factor IX protein is not a hyperactive factor IX variant; and (II) the second factor IX protein coding sequence is at least 95% identical to SEQ ID NO: 166, is CpG-depleted, and is codon-optimized, and optionally wherein the factor IX protein is not a hyperactive factor IX variant.

In some such compositions: (I) the first factor IX protein coding sequence is at least 99% identical to SEQ ID NO: 159 and is: (i) CpG-depleted; (ii) modified to mutate one or more cryptic splice donor sequences; or (iii) CpG-depleted and modified to mutate one or more cryptic splice donor sequences, and optionally wherein the factor IX protein is not a hyperactive factor IX variant; and (II) the second factor IX protein coding sequence is at least 95% identical to SEQ ID NO: 165, is CpG-depleted, and is codon-optimized, and optionally wherein the factor IX protein is not a hyperactive factor IX variant.

In some such compositions: (I) the first factor IX protein coding sequence is at least 99% identical to SEQ ID NO: 159, is CpG-depleted, and is modified to mutate one or more cryptic splice donor sequences, and optionally wherein the factor IX protein is not a hyperactive factor IX variant; and (II) the second factor IX protein coding sequence is at least 99% identical to SEQ ID NO: 166, is CpG-depleted, and is codon-optimized, and optionally wherein the factor IX protein is not a hyperactive factor IX variant.

In some such compositions: (I) the first factor IX protein coding sequence is at least 99% identical to SEQ ID NO: 159, is CpG-depleted, and is modified to mutate one or more cryptic splice donor sequences, and optionally wherein the factor IX protein is not a hyperactive factor IX variant; and (II) the second factor IX protein coding sequence is at least 99% identical to SEQ ID NO: 165, is CpG-depleted, and is codon-optimized, and optionally wherein the factor IX protein is not a hyperactive factor IX variant.

In some such compositions, the first factor IX protein coding sequence has all but one CpG dinucleotides removed or is fully CpG depleted and the second factor IX protein coding sequence has all but one CpG dinucleotides removed or is fully CpG depleted.

In some such compositions, the first factor IX protein coding sequence encodes a factor IX protein at least 99% identical to SEQ ID NO: 195 and optionally wherein the factor IX protein is not a hyperactive factor IX variant, and the second factor IX protein coding sequence encodes a factor IX protein at least 99% identical to SEQ ID NO: 195 and optionally wherein the factor IX protein is not a hyperactive factor IX variant. In some such compositions, the first factor IX protein coding sequence encodes a factor IX protein comprising SEQ ID NO: 195, and the second factor IX protein coding sequence encodes a factor IX protein comprising SEQ ID NO: 195.

In some such compositions: (I) the first factor IX protein coding sequence comprises SEQ ID NO: 159; and (II) the second factor IX protein coding sequence comprises SEQ ID NO: 166. In some such compositions: (I) the first factor IX protein coding sequence consists of SEQ ID NO: 159; and (II) the second factor IX protein coding sequence consists of SEQ ID NO: 166.

In some such compositions: (I) the first factor IX protein coding sequence comprises SEQ ID NO: 159; and (II) the second factor IX protein coding sequence comprises SEQ ID NO: 165. In some such compositions: (I) the first factor IX protein coding sequence consists of SEQ ID NO: 159; and (II) the second factor IX protein coding sequence consists of SEQ ID NO: 165.

In some such compositions, the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, or wherein the nucleic acid construct comprises from 5′ to 3′: a first splice acceptor, the second factor IX protein coding sequence, a first polyadenylation signal, a reverse complement of a second polyadenylation signal, the reverse complement of the first factor IX protein coding sequence, and a reverse complement of a second splice acceptor. In some such compositions, the first factor IX protein coding sequence and the second factor IX protein coding sequence are different but encode the same factor IX protein sequence, and wherein the first polyadenylation signal and the second polyadenylation signal are different.

In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the nucleic acid construct does not comprise homology arms.

In some such compositions, the nucleic acid construct comprises SEQ ID NO: 210 or 180 or the reverse complement thereof.

In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, and wherein the nucleic acid construct does not comprise homology arms.

In some such compositions, the nucleic acid construct comprises SEQ ID NO: 209 or 179 or the reverse complement thereof.

In some such compositions, the nucleic acid construct is single-stranded DNA or double-stranded DNA. In some such compositions, the nucleic acid construct is single-stranded DNA. In some such compositions, the nucleic acid construct is in a nucleic acid vector or a lipid nanoparticle. In some such compositions, the nucleic acid construct is in the nucleic acid vector. In some such compositions, the nucleic acid vector is a viral vector. In some such compositions, the nucleic acid vector is an adeno-associated viral (AAV) vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the AAV vector is a single-stranded AAV (ssAAV) vector. In some such compositions, the AAV vector is derived from an AAV8 vector, an AAV3B vector, an AAV5 vector, an AAV6 vector, an AAV7 vector, an AAV9 vector, an AAVrh.74 vector, or an AAVhu.37 vector. In some such compositions, the AAV vector is a recombinant AAV8 (rAAV8) vector. In some such compositions, the AAV vector is a single-stranded rAAV8 vector.

In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196.

In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196.

In some such compositions, the nucleic acid construct is a unidirectional construct. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166; wherein the nucleic acid construct does not comprise a promoter that drives the expression of the factor IX protein, and wherein the nucleic acid construct does not comprise homology arms. In some such compositions, the first factor IX protein coding sequence comprises SEQ ID NO: 166. In some such compositions, the first factor IX protein coding sequence comprises SEQ ID NO: 159.

In some such compositions, the nucleic acid construct is a unidirectional construct. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165; wherein the nucleic acid construct does not comprise a promoter that drives the expression of the factor IX protein, and wherein the nucleic acid construct does not comprise homology arms. In some such compositions, the first factor IX protein coding sequence comprises SEQ ID NO: 165. In some such compositions, the first factor IX protein coding sequence comprises SEQ ID NO: 159.

In some such compositions, the nucleic acid construct is single-stranded DNA or double-stranded DNA. In some such compositions, the nucleic acid construct is single-stranded DNA. In some such compositions, the nucleic acid construct is in a nucleic acid vector or a lipid nanoparticle. In some such compositions, the nucleic acid construct is in the nucleic acid vector. In some such compositions, the nucleic acid vector is a viral vector. In some such compositions, the nucleic acid vector is an adeno-associated viral (AAV) vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the AAV vector is a single-stranded AAV (ssAAV) vector. In some such compositions, the AAV vector is derived from an AAV8 vector, an AAV3B vector, an AAV5 vector, an AAV6 vector, an AAV7 vector, an AAV9 vector, an AAVrh.74 vector, or an AAVhu.37 vector. In some such compositions, the AAV vector is a recombinant AAV8 (rAAV8) vector. In some such compositions, the AAV vector is a single-stranded rAAV8 vector.

In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the first factor IX protein coding sequence comprises SEQ ID NO: 166. In some such compositions, the first factor IX protein coding sequence comprises SEQ ID NO: 159.

In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the first factor IX protein coding sequence comprises SEQ ID NO: 165. In some such compositions, the first factor IX protein coding sequence comprises SEQ ID NO: 159.

Some such compositions further comprise a nuclease agent that targets a nuclease target site in a target gene. In some such compositions, the target gene is an albumin gene, optionally wherein the albumin gene is a human albumin gene. In some such compositions, the nuclease agent comprises: (a) a zinc finger nuclease (ZFN); (b) a transcription activator-like effector nuclease (TALEN); or (c) (i) a Cas protein or a nucleic acid encoding the Cas protein; and (ii) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence. In some such compositions, the nuclease agent comprises: (a) a Cas protein or a nucleic acid encoding the Cas protein; and (b) a guide RNA or one or more DNAs encoding the guide RNA, wherein the guide RNA comprises a DNA-targeting segment that targets a guide RNA target sequence in intron 1 of an albumin gene, and wherein the guide RNA binds to the Cas protein and targets the Cas protein to the guide RNA target sequence. In some such compositions, the albumin gene is a human albumin gene.

In some such compositions: (I) the DNA-targeting segment comprises at least 17, at least 18, at least 19, or at least 20 contiguous nucleotides of the sequence set forth in any one of SEQ ID NOS: 30-61, optionally wherein the DNA-targeting segment comprises at least 17, at least 18, at least 19, or at least 20 contiguous nucleotides of the sequence set forth in any one of SEQ ID NOS: 36, 30, 33, and 41; and/or (II) the DNA-targeting segment is at least 90% or at least 95% identical to the sequence set forth in any one of SEQ ID NOS: 30-61, optionally wherein the DNA-targeting segment is at least 90% or at least 95% identical to the sequence set forth in any one of SEQ ID NOS: 36, 30, 33, and 41. In some such compositions, the DNA-targeting segment comprises any one of SEQ ID NOS: 30-61, optionally wherein the DNA-targeting segment comprises any one of SEQ ID NOS: 36, 30, 33, and 41. In some such compositions, the DNA-targeting segment consists of any one of SEQ ID NOS: 30-61, optionally wherein the DNA-targeting segment consists of any one of SEQ ID NOS: 36, 30, 33, and 41. In some such compositions, the guide RNA comprises any one of SEQ ID NOS: 62-125, optionally wherein the guide RNA comprises any one of SEQ ID NOS: 68, 100, 62, 94, 65, 97, 73, and 105.

In some such compositions: (I) the DNA-targeting segment comprises at least 17, at least 18, at least 19, or at least 20 contiguous nucleotides of SEQ ID NO: 36; and/or (II) the DNA-targeting segment is at least 90% or at least 95% identical to SEQ ID NO: 36. In some such compositions, the DNA-targeting segment comprises SEQ ID NO: 36. In some such compositions, the DNA-targeting segment consists of SEQ ID NO: 36. In some such compositions, the guide RNA comprises SEQ ID NO: 68 or 100.

In some such compositions: (I) the DNA-targeting segment comprises at least 17, at least 18, at least 19, or at least 20 contiguous nucleotides of SEQ ID NO: 30; and/or (II) the DNA-targeting segment is at least 90% or at least 95% identical to SEQ ID NO: 30. In some such compositions, the DNA-targeting segment comprises SEQ ID NO: 30. In some such compositions, the DNA-targeting segment consists of SEQ ID NO: 30. In some such compositions, the guide RNA comprises SEQ ID NO: 62 or 94.

In some such compositions: (I) the DNA-targeting segment comprises at least 17, at least 18, at least 19, or at least 20 contiguous nucleotides of SEQ ID NO: 33; and/or (II) the DNA-targeting segment is at least 90% or at least 95% identical to SEQ ID NO: 33. In some such compositions, the DNA-targeting segment comprises SEQ ID NO: 33. In some such compositions, the DNA-targeting segment consists of SEQ ID NO: 33. In some such compositions, the guide RNA comprises SEQ ID NO: 65 or 97.

In some such compositions: (I) the DNA-targeting segment comprises at least 17, at least 18, at least 19, or at least 20 contiguous nucleotides of SEQ ID NO: 41; and/or (II) the DNA-targeting segment is at least 90% or at least 95% identical to SEQ ID NO: 41. In some such compositions, the DNA-targeting segment comprises SEQ ID NO: 41. In some such compositions, the DNA-targeting segment consists of SEQ ID NO: 41. In some such compositions, the guide RNA comprises SEQ ID NO: 73 or 105.

In some such compositions, the composition comprises the guide RNA in the form of RNA. In some such compositions, the guide RNA comprises at least one modification. In some such compositions, the at least one modification comprises a 2′-O-methyl-modified nucleotide. In some such compositions, the at least one modification comprises a phosphorothioate bond between nucleotides. In some such compositions, the at least one modification comprises a modification at one or more of the first five nucleotides at the 5′ end of the guide RNA. In some such compositions, the at least one modification comprises a modification at one or more of the last five nucleotides at the 3′ end of the guide RNA. In some such compositions, the at least one modification comprises phosphorothioate bonds between the first four nucleotides at the 5′ end of the guide RNA. In some such compositions, the at least one modification comprises phosphorothioate bonds between the last four nucleotides at the 3′ end of the guide RNA. In some such compositions, the at least one modification comprises 2′-O-methyl-modified nucleotides at the first three nucleotides at the 5′ end of the guide RNA. In some such compositions, the at least one modification comprises 2′-O-methyl-modified nucleotides at the last three nucleotides at the 3′ end of the guide RNA. In some such compositions, the at least one modification comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

In some such compositions, the guide RNA is a single guide RNA (sgRNA). In some such compositions, the composition comprises the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

In some such compositions, the guide RNA is a single guide RNA (sgRNA). In some such compositions, the composition comprises the guide RNA in the form of RNA, the guide RNA comprises SEQ ID NO: 94, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

In some such compositions, the guide RNA is a single guide RNA (sgRNA). In some such compositions, the composition comprises the guide RNA in the form of RNA, the guide RNA comprises SEQ ID NO: 97, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

In some such compositions, the guide RNA is a single guide RNA (sgRNA). In some such compositions, the composition comprises the guide RNA in the form of RNA, the guide RNA comprises SEQ ID NO: 105, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

In some such compositions, the Cas protein is a Cas9 protein. In some such compositions, the Cas protein is or is derived from a Streptococcus pyogenes Cas9 protein. In some such compositions, the Cas protein comprises the sequence set forth in SEQ ID NO: 11. In some such compositions, the nucleic acid encoding the Cas protein is codon-optimized for expression in a mammalian cell or a human cell. In some such compositions, the composition comprises the nucleic acid encoding the Cas protein, wherein the nucleic acid comprises an mRNA encoding the Cas protein. In some such compositions, the mRNA encoding the Cas protein comprises at least one modification. In some such compositions, the mRNA encoding the Cas protein is modified to comprise a modified uridine at one or more or all uridine positions. In some such compositions, the modified uridine is pseudouridine or N1-methyl-pseudouridine, optionally N1-methyl-pseudouridine. In some such compositions, the mRNA encoding the Cas protein is fully substituted with pseudouridine or N1-methyl-pseudouridine, optionally N1-methyl-pseudouridine. In some such compositions, the modified uridine is pseudouridine. In some such compositions, the mRNA encoding the Cas protein is fully substituted with pseudouridine. In some such compositions, the mRNA encoding the Cas protein comprises a 5′ cap. In some such compositions, the mRNA encoding the Cas protein comprises a poly(A) tail. In some such compositions, the mRNA encoding the Cas protein comprises the sequence set forth in SEQ ID NO: 12. In some such compositions, the mRNA encoding the Cas protein comprises the sequence set forth in SEQ ID NO: 225. In some such compositions, the mRNA encoding the Cas protein comprises the sequence set forth in SEQ ID NO: 226.

In some such compositions, the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine or N1-methyl-pseudouridine, optionally N1-methyl-pseudouridine, comprises a 5′ cap, and comprises a poly(A) tail. In some such compositions, the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine, comprises a 5′ cap, and comprises a poly(A) tail.

In some such compositions, the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine or N1-methyl-pseudouridine, optionally N1-methyl-pseudouridine, comprises a 5′ cap, and comprises a poly(A) tail. In some such compositions, the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine, comprises a 5′ cap, and comprises a poly(A) tail.

In some such compositions, the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine or N1-methyl-pseudouridine, optionally N1-methyl-pseudouridine, comprises a 5′ cap, and comprises a poly(A) tail. In some such compositions, the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine, comprises a 5′ cap, and comprises a poly(A) tail.

In some such compositions, the composition comprises the guide RNA in the form of RNA, and the guide RNA comprises SEQ ID NO: 68 or 100, and wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 12. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 166. Optionally, the first factor IX protein coding sequence comprises SEQ ID NO: 159.

In some such compositions, the composition comprises the guide RNA in the form of RNA, and the guide RNA comprises SEQ ID NO: 68 or 100, and wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 12. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 165. Optionally, the first factor IX protein coding sequence comprises SEQ ID NO: 159.

In some such compositions, the composition comprises the guide RNA in the form of RNA, and the guide RNA comprises SEQ ID NO: 62 or 94, and wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 12. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 166. Optionally, the first factor IX protein coding sequence comprises SEQ ID NO: 159.

In some such compositions, the composition comprises the guide RNA in the form of RNA, and the guide RNA comprises SEQ ID NO: 62 or 94, and wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 12. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 165. Optionally, the first factor IX protein coding sequence comprises SEQ ID NO: 159.

In some such compositions, the composition comprises the guide RNA in the form of RNA, and the guide RNA comprises SEQ ID NO: 65 or 97, and wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 12. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 166. Optionally, the first factor IX protein coding sequence comprises SEQ ID NO: 159.

In some such compositions, the composition comprises the guide RNA in the form of RNA, and the guide RNA comprises SEQ ID NO: 65 or 97, and wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 12. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 165. Optionally, the first factor IX protein coding sequence comprises SEQ ID NO: 159.

In some such compositions, the composition comprises the guide RNA in the form of RNA, and the guide RNA comprises SEQ ID NO: 73 or 105, and wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 12. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 166. Optionally, the first factor IX protein coding sequence comprises SEQ ID NO: 159.

In some such compositions, the composition comprises the guide RNA in the form of RNA, and the guide RNA comprises SEQ ID NO: 73 or 105, and wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 12. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 165. Optionally, the first factor IX protein coding sequence comprises SEQ ID NO: 159.

In some such compositions, the composition comprises the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine or N1-methyl-pseudouridine, optionally N1-methyl-pseudouridine, comprises a 5′ cap, and comprises a poly(A) tail. In some such compositions, the composition comprises the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine, comprises a 5′ cap, and comprises a poly(A) tail. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 166. Optionally, the first factor IX protein coding sequence comprises SEQ ID NO: 159.

In some such compositions, the composition comprises the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine or N1-methyl-pseudouridine, optionally N1-methyl-pseudouridine, comprises a 5′ cap, and comprises a poly(A) tail. In some such compositions, the composition comprises the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine, comprises a 5′ cap, and comprises a poly(A) tail. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 165. Optionally, the first factor IX protein coding sequence comprises SEQ ID NO: 159.

In some such compositions, the composition comprises the guide RNA in the form of RNA, the guide RNA comprises SEQ ID NO: 94, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine or N1-methyl-pseudouridine, optionally N1-methyl-pseudouridine, comprises a 5′ cap, and comprises a poly(A) tail. In some such compositions, the composition comprises the guide RNA in the form of RNA, the guide RNA comprises SEQ ID NO: 94, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine, comprises a 5′ cap, and comprises a poly(A) tail. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 166. Optionally, the first factor IX protein coding sequence comprises SEQ ID NO: 159.

In some such compositions, the composition comprises the guide RNA in the form of RNA, the guide RNA comprises SEQ ID NO: 94, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine or N1-methyl-pseudouridine, optionally N1-methyl-pseudouridine, comprises a 5′ cap, and comprises a poly(A) tail. In some such compositions, the composition comprises the guide RNA in the form of RNA, the guide RNA comprises SEQ ID NO: 94, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine, comprises a 5′ cap, and comprises a poly(A) tail. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 165. Optionally, the first factor IX protein coding sequence comprises SEQ ID NO: 159.

In some such compositions, the composition comprises the guide RNA in the form of RNA, the guide RNA comprises SEQ ID NO: 97, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine or N1-methyl-pseudouridine, optionally N1-methyl-pseudouridine, comprises a 5′ cap, and comprises a poly(A) tail. In some such compositions, the composition comprises the guide RNA in the form of RNA, the guide RNA comprises SEQ ID NO: 97, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine, comprises a 5′ cap, and comprises a poly(A) tail. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 166. Optionally, the first factor IX protein coding sequence comprises SEQ ID NO: 159.

In some such compositions, the composition comprises the guide RNA in the form of RNA, the guide RNA comprises SEQ ID NO: 97, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine or N1-methyl-pseudouridine, optionally N1-methyl-pseudouridine, comprises a 5′ cap, and comprises a poly(A) tail. In some such compositions, the composition comprises the guide RNA in the form of RNA, the guide RNA comprises SEQ ID NO: 97, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine, comprises a 5′ cap, and comprises a poly(A) tail. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 165. Optionally, the first factor IX protein coding sequence comprises SEQ ID NO: 159.

In some such compositions, the composition comprises the guide RNA in the form of RNA, the guide RNA comprises SEQ ID NO: 105, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine or N1-methyl-pseudouridine, optionally N1-methyl-pseudouridine, comprises a 5′ cap, and comprises a poly(A) tail. In some such compositions, the composition comprises the guide RNA in the form of RNA, the guide RNA comprises SEQ ID NO: 105, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine, comprises a 5′ cap, and comprises a poly(A) tail. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 166. Optionally, the first factor IX protein coding sequence comprises SEQ ID NO: 159.

In some such compositions, the composition comprises the guide RNA in the form of RNA, the guide RNA comprises SEQ ID NO: 105, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine or N1-methyl-pseudouridine, optionally N1-methyl-pseudouridine, comprises a 5′ cap, and comprises a poly(A) tail. In some such compositions, the composition comprises the guide RNA in the form of RNA, the guide RNA comprises SEQ ID NO: 105, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine, comprises a 5′ cap, and comprises a poly(A) tail. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 165. Optionally, the first factor IX protein coding sequence comprises SEQ ID NO: 159.

In some such compositions, the composition comprises the guide RNA in the form of RNA, and the guide RNA comprises SEQ ID NO: 68 or 100, and wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 225. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 166. Optionally, the first factor IX protein coding sequence comprises SEQ ID NO: 159.

In some such compositions, the composition comprises the guide RNA in the form of RNA, and the guide RNA comprises SEQ ID NO: 68 or 100, and wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 225. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 165. Optionally, the first factor IX protein coding sequence comprises SEQ ID NO: 159.

In some such compositions, the composition comprises the guide RNA in the form of RNA, and the guide RNA comprises SEQ ID NO: 62 or 94, and wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 225. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 166. Optionally, the first factor IX protein coding sequence comprises SEQ ID NO: 159.

In some such compositions, the composition comprises the guide RNA in the form of RNA, and the guide RNA comprises SEQ ID NO: 62 or 94, and wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 225. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 165. Optionally, the first factor IX protein coding sequence comprises SEQ ID NO: 159.

In some such compositions, the composition comprises the guide RNA in the form of RNA, and the guide RNA comprises SEQ ID NO: 65 or 97, and wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 225. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 166. Optionally, the first factor IX protein coding sequence comprises SEQ ID NO: 159.

In some such compositions, the composition comprises the guide RNA in the form of RNA, and the guide RNA comprises SEQ ID NO: 65 or 97, and wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 225. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 165. Optionally, the first factor IX protein coding sequence comprises SEQ ID NO: 159.

In some such compositions, the composition comprises the guide RNA in the form of RNA, and the guide RNA comprises SEQ ID NO: 73 or 105, and wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 225. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 166. Optionally, the first factor IX protein coding sequence comprises SEQ ID NO: 159.

In some such compositions, the composition comprises the guide RNA in the form of RNA, and the guide RNA comprises SEQ ID NO: 73 or 105, and wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 225. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 165. Optionally, the first factor IX protein coding sequence comprises SEQ ID NO: 159.

In some such compositions, the composition comprises the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine or N1-methyl-pseudouridine, optionally N1-methyl-pseudouridine, comprises a 5′ cap, and comprises a poly(A) tail. In some such compositions, the composition comprises the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine, comprises a 5′ cap, and comprises a poly(A) tail. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 166. Optionally, the first factor IX protein coding sequence comprises SEQ ID NO: 159.

In some such compositions, the composition comprises the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine or N1-methyl-pseudouridine, optionally N1-methyl-pseudouridine, comprises a 5′ cap, and comprises a poly(A) tail. In some such compositions, the composition comprises the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine, comprises a 5′ cap, and comprises a poly(A) tail. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 165. Optionally, the first factor IX protein coding sequence comprises SEQ ID NO: 159.

In some such compositions, the composition comprises the guide RNA in the form of RNA, the guide RNA comprises SEQ ID NO: 94, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine or N1-methyl-pseudouridine, optionally N1-methyl-pseudouridine, comprises a 5′ cap, and comprises a poly(A) tail. In some such compositions, the composition comprises the guide RNA in the form of RNA, the guide RNA comprises SEQ ID NO: 94, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine, comprises a 5′ cap, and comprises a poly(A) tail. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 166. Optionally, the first factor IX protein coding sequence comprises SEQ ID NO: 159.

In some such compositions, the composition comprises the guide RNA in the form of RNA, the guide RNA comprises SEQ ID NO: 94, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine or N1-methyl-pseudouridine, optionally N1-methyl-pseudouridine, comprises a 5′ cap, and comprises a poly(A) tail. In some such compositions, the composition comprises the guide RNA in the form of RNA, the guide RNA comprises SEQ ID NO: 94, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine, comprises a 5′ cap, and comprises a poly(A) tail. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 165. Optionally, the first factor IX protein coding sequence comprises SEQ ID NO: 159.

In some such compositions, the composition comprises the guide RNA in the form of RNA, the guide RNA comprises SEQ ID NO: 97, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine or N1-methyl-pseudouridine, optionally N1-methyl-pseudouridine, comprises a 5′ cap, and comprises a poly(A) tail. In some such compositions, the composition comprises the guide RNA in the form of RNA, the guide RNA comprises SEQ ID NO: 97, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine, comprises a 5′ cap, and comprises a poly(A) tail. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 166. Optionally, the first factor IX protein coding sequence comprises SEQ ID NO: 159.

In some such compositions, the composition comprises the guide RNA in the form of RNA, the guide RNA comprises SEQ ID NO: 97, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine or N1-methyl-pseudouridine, optionally N1-methyl-pseudouridine, comprises a 5′ cap, and comprises a poly(A) tail. In some such compositions, the composition comprises the guide RNA in the form of RNA, the guide RNA comprises SEQ ID NO: 97, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine, comprises a 5′ cap, and comprises a poly(A) tail. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 165. Optionally, the first factor IX protein coding sequence comprises SEQ ID NO: 159.

In some such compositions, the composition comprises the guide RNA in the form of RNA, the guide RNA comprises SEQ ID NO: 105, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine or N1-methyl-pseudouridine, optionally N1-methyl-pseudouridine, comprises a 5′ cap, and comprises a poly(A) tail. In some such compositions, the composition comprises the guide RNA in the form of RNA, the guide RNA comprises SEQ ID NO: 105, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine, comprises a 5′ cap, and comprises a poly(A) tail. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 166. Optionally, the first factor IX protein coding sequence comprises SEQ ID NO: 159.

In some such compositions, the composition comprises the guide RNA in the form of RNA, the guide RNA comprises SEQ ID NO: 105, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine or N1-methyl-pseudouridine, optionally N1-methyl-pseudouridine, comprises a 5′ cap, and comprises a poly(A) tail. In some such compositions, the composition comprises the guide RNA in the form of RNA, the guide RNA comprises SEQ ID NO: 105, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine, comprises a 5′ cap, and comprises a poly(A) tail. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 165. Optionally, the first factor IX protein coding sequence comprises SEQ ID NO: 159.

In some such compositions, the composition comprises the guide RNA in the form of RNA, and the guide RNA comprises SEQ ID NO: 68 or 100, and wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 226. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 166. Optionally, the first factor IX protein coding sequence comprises SEQ ID NO: 159.

In some such compositions, the composition comprises the guide RNA in the form of RNA, and the guide RNA comprises SEQ ID NO: 68 or 100, and wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 226. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 165. Optionally, the first factor IX protein coding sequence comprises SEQ ID NO: 159.

In some such compositions, the composition comprises the guide RNA in the form of RNA, and the guide RNA comprises SEQ ID NO: 62 or 94, and wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 226. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 166. Optionally, the first factor IX protein coding sequence comprises SEQ ID NO: 159.

In some such compositions, the composition comprises the guide RNA in the form of RNA, and the guide RNA comprises SEQ ID NO: 62 or 94, and wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 226. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 165. Optionally, the first factor IX protein coding sequence comprises SEQ ID NO: 159.

In some such compositions, the composition comprises the guide RNA in the form of RNA, and the guide RNA comprises SEQ ID NO: 65 or 97, and wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 226. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 166. Optionally, the first factor IX protein coding sequence comprises SEQ ID NO: 159.

In some such compositions, the composition comprises the guide RNA in the form of RNA, and the guide RNA comprises SEQ ID NO: 65 or 97, and wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 226. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 165. Optionally, the first factor IX protein coding sequence comprises SEQ ID NO: 159.

In some such compositions, the composition comprises the guide RNA in the form of RNA, and the guide RNA comprises SEQ ID NO: 73 or 105, and wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 226. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 166. Optionally, the first factor IX protein coding sequence comprises SEQ ID NO: 159.

In some such compositions, the composition comprises the guide RNA in the form of RNA, and the guide RNA comprises SEQ ID NO: 73 or 105, and wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 226. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 165. Optionally, the first factor IX protein coding sequence comprises SEQ ID NO: 159.

In some such compositions, the composition comprises the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine or N1-methyl-pseudouridine, optionally N1-methyl-pseudouridine, comprises a 5′ cap, and comprises a poly(A) tail. In some such compositions, the composition comprises the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine, comprises a 5′ cap, and comprises a poly(A) tail. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 166. Optionally, the first factor IX protein coding sequence comprises SEQ ID NO: 159.

In some such compositions, the composition comprises the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine or N1-methyl-pseudouridine, optionally N1-methyl-pseudouridine, comprises a 5′ cap, and comprises a poly(A) tail. In some such compositions, the composition comprises the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine, comprises a 5′ cap, and comprises a poly(A) tail. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 165. Optionally, the first factor IX protein coding sequence comprises SEQ ID NO: 159.

In some such compositions, the composition comprises the guide RNA in the form of RNA, the guide RNA comprises SEQ ID NO: 94, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine or N1-methyl-pseudouridine, optionally N1-methyl-pseudouridine, comprises a 5′ cap, and comprises a poly(A) tail. In some such compositions, the composition comprises the guide RNA in the form of RNA, the guide RNA comprises SEQ ID NO: 94, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine, comprises a 5′ cap, and comprises a poly(A) tail. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 166. Optionally, the first factor IX protein coding sequence comprises SEQ ID NO: 159.

In some such compositions, the composition comprises the guide RNA in the form of RNA, the guide RNA comprises SEQ ID NO: 94, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine or N1-methyl-pseudouridine, optionally N1-methyl-pseudouridine, comprises a 5′ cap, and comprises a poly(A) tail. In some such compositions, the composition comprises the guide RNA in the form of RNA, the guide RNA comprises SEQ ID NO: 94, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine, comprises a 5′ cap, and comprises a poly(A) tail. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 165. Optionally, the first factor IX protein coding sequence comprises SEQ ID NO: 159.

In some such compositions, the composition comprises the guide RNA in the form of RNA, the guide RNA comprises SEQ ID NO: 97, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine or N1-methyl-pseudouridine, optionally N1-methyl-pseudouridine, comprises a 5′ cap, and comprises a poly(A) tail. In some such compositions, the composition comprises the guide RNA in the form of RNA, the guide RNA comprises SEQ ID NO: 97, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine, comprises a 5′ cap, and comprises a poly(A) tail. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 166. Optionally, the first factor IX protein coding sequence comprises SEQ ID NO: 159.

In some such compositions, the composition comprises the guide RNA in the form of RNA, the guide RNA comprises SEQ ID NO: 97, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine or N1-methyl-pseudouridine, optionally N1-methyl-pseudouridine, comprises a 5′ cap, and comprises a poly(A) tail. In some such compositions, the composition comprises the guide RNA in the form of RNA, the guide RNA comprises SEQ ID NO: 97, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine, comprises a 5′ cap, and comprises a poly(A) tail. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 165. Optionally, the first factor IX protein coding sequence comprises SEQ ID NO: 159.

In some such compositions, the composition comprises the guide RNA in the form of RNA, the guide RNA comprises SEQ ID NO: 105, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine or N1-methyl-pseudouridine, optionally N1-methyl-pseudouridine, comprises a 5′ cap, and comprises a poly(A) tail. In some such compositions, the composition comprises the guide RNA in the form of RNA, the guide RNA comprises SEQ ID NO: 105, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine, comprises a 5′ cap, and comprises a poly(A) tail. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 166. Optionally, the first factor IX protein coding sequence comprises SEQ ID NO: 159.

In some such compositions, the composition comprises the guide RNA in the form of RNA, the guide RNA comprises SEQ ID NO: 105, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine or N1-methyl-pseudouridine, optionally N1-methyl-pseudouridine, comprises a 5′ cap, and comprises a poly(A) tail. In some such compositions, the composition comprises the guide RNA in the form of RNA, the guide RNA comprises SEQ ID NO: 105, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine, comprises a 5′ cap, and comprises a poly(A) tail. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, and wherein the nucleic acid construct does not comprise homology arms. Optionally, the first factor IX protein coding sequence comprises SEQ ID NO: 165. Optionally, the first factor IX protein coding sequence comprises SEQ ID NO: 159.

In some such compositions, the Cas protein or the nucleic acid encoding the Cas protein and the guide RNA or the one or more DNAs encoding the guide RNA are associated with a lipid nanoparticle. In some such compositions, the lipid nanoparticle comprises a cationic lipid, a neutral lipid, a helper lipid, and a stealth lipid. In some such compositions, the cationic lipid is Lipid A. In some such compositions, the neutral lipid is DSPC. In some such compositions, the helper lipid is cholesterol. In some such compositions, the stealth lipid is 1,2-dimyristoyl-rac-glycero-3-methoxypolyethylene glycol-2000 (PEG2k-DMG). In some such compositions, the cationic lipid is Lipid A, the neutral lipid is DSPC, the helper lipid is cholesterol, and the stealth lipid is PEG2k-DMG. In some such compositions, the lipid nanoparticle comprises four lipids at the following molar ratios: about 50 mol % Lipid A, about 9 mol % DSPC, about 38 mol % cholesterol, and about 3 mol % PEG2k-DMG.

In some such compositions, the albumin gene is a human albumin gene, wherein the composition comprises the guide RNA in the form of RNA, and the guide RNA comprises SEQ ID NO: 68 or 100, wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 12, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 166. Optionally, the first factor IX protein coding sequence comprises SEQ ID NO: 159.

In some such compositions, the albumin gene is a human albumin gene, wherein the composition comprises the guide RNA in the form of RNA, and the guide RNA comprises SEQ ID NO: 68 or 100, wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 12, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 165. Optionally, the first factor IX protein coding sequence comprises SEQ ID NO: 159.

In some such compositions, the albumin gene is a human albumin gene, wherein the composition comprises the guide RNA in the form of RNA, and the guide RNA comprises SEQ ID NO: 62 or 94, wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 12, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 166. Optionally, the first factor IX protein coding sequence comprises SEQ ID NO: 159.

In some such compositions, the albumin gene is a human albumin gene, wherein the composition comprises the guide RNA in the form of RNA, and the guide RNA comprises SEQ ID NO: 62 or 94, wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 12, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 165. Optionally, the first factor IX protein coding sequence comprises SEQ ID NO: 159.

In some such compositions, the albumin gene is a human albumin gene, wherein the composition comprises the guide RNA in the form of RNA, and the guide RNA comprises SEQ ID NO: 65 or 97, wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 12, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 166. Optionally, the first factor IX protein coding sequence comprises SEQ ID NO: 159.

In some such compositions, the albumin gene is a human albumin gene, wherein the composition comprises the guide RNA in the form of RNA, and the guide RNA comprises SEQ ID NO: 65 or 97, wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 12, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 165. Optionally, the first factor IX protein coding sequence comprises SEQ ID NO: 159.

In some such compositions, the albumin gene is a human albumin gene, wherein the composition comprises the guide RNA in the form of RNA, and the guide RNA comprises SEQ ID NO: 73 or 105, wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 12, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 166. Optionally, the first factor IX protein coding sequence comprises SEQ ID NO: 159.

In some such compositions, the albumin gene is a human albumin gene, wherein the composition comprises the guide RNA in the form of RNA, and the guide RNA comprises SEQ ID NO: 73 or 105, wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 12, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 165. Optionally, the first factor IX protein coding sequence comprises SEQ ID NO: 159.

In some such compositions, the albumin gene is a human albumin gene, wherein the composition comprises the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine or N1-methyl-pseudouridine, optionally N1-methyl-pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the albumin gene is a human albumin gene, wherein the composition comprises the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 166. Optionally, the first factor IX protein coding sequence comprises SEQ ID NO: 159.

In some such compositions, the albumin gene is a human albumin gene, wherein the composition comprises the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine or N1-methyl-pseudouridine, optionally N1-methyl-pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the albumin gene is a human albumin gene, wherein the composition comprises the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 165. Optionally, the first factor IX protein coding sequence comprises SEQ ID NO: 159.

In some such compositions, the albumin gene is a human albumin gene, wherein the composition comprises the guide RNA in the form of RNA, the guide RNA comprises SEQ ID NO: 94, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine or N1-methyl-pseudouridine, optionally N1-methyl-pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the albumin gene is a human albumin gene, wherein the composition comprises the guide RNA in the form of RNA, the guide RNA comprises SEQ ID NO: 94, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 166. Optionally, the first factor IX protein coding sequence comprises SEQ ID NO: 159.

In some such compositions, the albumin gene is a human albumin gene, wherein the composition comprises the guide RNA in the form of RNA, the guide RNA comprises SEQ ID NO: 94, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine or N1-methyl-pseudouridine, optionally N1-methyl-pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the albumin gene is a human albumin gene, wherein the composition comprises the guide RNA in the form of RNA, the guide RNA comprises SEQ ID NO: 94, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 165. Optionally, the first factor IX protein coding sequence comprises SEQ ID NO: 159.

In some such compositions, the albumin gene is a human albumin gene, wherein the composition comprises the guide RNA in the form of RNA, the guide RNA comprises SEQ ID NO: 97, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine or N1-methyl-pseudouridine, optionally N1-methyl-pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the albumin gene is a human albumin gene, wherein the composition comprises the guide RNA in the form of RNA, the guide RNA comprises SEQ ID NO: 97, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 166. Optionally, the first factor IX protein coding sequence comprises SEQ ID NO: 159.

In some such compositions, the albumin gene is a human albumin gene, wherein the composition comprises the guide RNA in the form of RNA, the guide RNA comprises SEQ ID NO: 97, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine or N1-methyl-pseudouridine, optionally N1-methyl-pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the albumin gene is a human albumin gene, wherein the composition comprises the guide RNA in the form of RNA, the guide RNA comprises SEQ ID NO: 97, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 165. Optionally, the first factor IX protein coding sequence comprises SEQ ID NO: 159.

In some such compositions, the albumin gene is a human albumin gene, wherein the composition comprises the guide RNA in the form of RNA, the guide RNA comprises SEQ ID NO: 105, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine or N1-methyl-pseudouridine, optionally N1-methyl-pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the albumin gene is a human albumin gene, wherein the composition comprises the guide RNA in the form of RNA, the guide RNA comprises SEQ ID NO: 105, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 166. Optionally, the first factor IX protein coding sequence comprises SEQ ID NO: 159.

In some such compositions, the albumin gene is a human albumin gene, wherein the composition comprises the guide RNA in the form of RNA, the guide RNA comprises SEQ ID NO: 105, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine or N1-methyl-pseudouridine, optionally N1-methyl-pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the albumin gene is a human albumin gene, wherein the composition comprises the guide RNA in the form of RNA, the guide RNA comprises SEQ ID NO: 105, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 12, and the mRNA encoding the Cas protein is fully substituted with pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 165. Optionally, the first factor IX protein coding sequence comprises SEQ ID NO: 159.

In some such compositions, the albumin gene is a human albumin gene, wherein the composition comprises the guide RNA in the form of RNA, and the guide RNA comprises SEQ ID NO: 68 or 100, wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 225, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 166. Optionally, the first factor IX protein coding sequence comprises SEQ ID NO: 159.

In some such compositions, the albumin gene is a human albumin gene, wherein the composition comprises the guide RNA in the form of RNA, and the guide RNA comprises SEQ ID NO: 68 or 100, wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 225, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 165. Optionally, the first factor IX protein coding sequence comprises SEQ ID NO: 159.

In some such compositions, the albumin gene is a human albumin gene, wherein the composition comprises the guide RNA in the form of RNA, and the guide RNA comprises SEQ ID NO: 62 or 94, wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 225, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 166. Optionally, the first factor IX protein coding sequence comprises SEQ ID NO: 159.

In some such compositions, the albumin gene is a human albumin gene, wherein the composition comprises the guide RNA in the form of RNA, and the guide RNA comprises SEQ ID NO: 62 or 94, wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 225, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 165. Optionally, the first factor IX protein coding sequence comprises SEQ ID NO: 159.

In some such compositions, the albumin gene is a human albumin gene, wherein the composition comprises the guide RNA in the form of RNA, and the guide RNA comprises SEQ ID NO: 65 or 97, wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 225, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 166. Optionally, the first factor IX protein coding sequence comprises SEQ ID NO: 159.

In some such compositions, the albumin gene is a human albumin gene, wherein the composition comprises the guide RNA in the form of RNA, and the guide RNA comprises SEQ ID NO: 65 or 97, wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 225, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 165. Optionally, the first factor IX protein coding sequence comprises SEQ ID NO: 159.

In some such compositions, the albumin gene is a human albumin gene, wherein the composition comprises the guide RNA in the form of RNA, and the guide RNA comprises SEQ ID NO: 73 or 105, wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 225, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 166. Optionally, the first factor IX protein coding sequence comprises SEQ ID NO: 159.

In some such compositions, the albumin gene is a human albumin gene, wherein the composition comprises the guide RNA in the form of RNA, and the guide RNA comprises SEQ ID NO: 73 or 105, wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 225, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 165. Optionally, the first factor IX protein coding sequence comprises SEQ ID NO: 159.

In some such compositions, the albumin gene is a human albumin gene, wherein the composition comprises the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine or N1-methyl-pseudouridine, optionally N1-methyl-pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the albumin gene is a human albumin gene, wherein the composition comprises the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 166. Optionally, the first factor IX protein coding sequence comprises SEQ ID NO: 159.

In some such compositions, the albumin gene is a human albumin gene, wherein the composition comprises the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine or N1-methyl-pseudouridine, optionally N1-methyl-pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the albumin gene is a human albumin gene, wherein the composition comprises the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 165. Optionally, the first factor IX protein coding sequence comprises SEQ ID NO: 159.

In some such compositions, the albumin gene is a human albumin gene, wherein the composition comprises the guide RNA in the form of RNA, the guide RNA comprises SEQ ID NO: 94, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine or N1-methyl-pseudouridine, optionally N1-methyl-pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the albumin gene is a human albumin gene, wherein the composition comprises the guide RNA in the form of RNA, the guide RNA comprises SEQ ID NO: 94, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 166. Optionally, the first factor IX protein coding sequence comprises SEQ ID NO: 159.

In some such compositions, the albumin gene is a human albumin gene, wherein the composition comprises the guide RNA in the form of RNA, the guide RNA comprises SEQ ID NO: 94, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine or N1-methyl-pseudouridine, optionally N1-methyl-pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the albumin gene is a human albumin gene, wherein the composition comprises the guide RNA in the form of RNA, the guide RNA comprises SEQ ID NO: 94, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 165. Optionally, the first factor IX protein coding sequence comprises SEQ ID NO: 159.

In some such compositions, the albumin gene is a human albumin gene, wherein the composition comprises the guide RNA in the form of RNA, the guide RNA comprises SEQ ID NO: 97, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine or N1-methyl-pseudouridine, optionally N1-methyl-pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the albumin gene is a human albumin gene, wherein the composition comprises the guide RNA in the form of RNA, the guide RNA comprises SEQ ID NO: 97, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 166. Optionally, the first factor IX protein coding sequence comprises SEQ ID NO: 159.

In some such compositions, the albumin gene is a human albumin gene, wherein the composition comprises the guide RNA in the form of RNA, the guide RNA comprises SEQ ID NO: 97, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine or N1-methyl-pseudouridine, optionally N1-methyl-pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the albumin gene is a human albumin gene, wherein the composition comprises the guide RNA in the form of RNA, the guide RNA comprises SEQ ID NO: 97, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 165. Optionally, the first factor IX protein coding sequence comprises SEQ ID NO: 159.

In some such compositions, the albumin gene is a human albumin gene, wherein the composition comprises the guide RNA in the form of RNA, the guide RNA comprises SEQ ID NO: 105, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine or N1-methyl-pseudouridine, optionally N1-methyl-pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the albumin gene is a human albumin gene, wherein the composition comprises the guide RNA in the form of RNA, the guide RNA comprises SEQ ID NO: 105, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 166. Optionally, the first factor IX protein coding sequence comprises SEQ ID NO: 159.

In some such compositions, the albumin gene is a human albumin gene, wherein the composition comprises the guide RNA in the form of RNA, the guide RNA comprises SEQ ID NO: 105, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine or N1-methyl-pseudouridine, optionally N1-methyl-pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the albumin gene is a human albumin gene, wherein the composition comprises the guide RNA in the form of RNA, the guide RNA comprises SEQ ID NO: 105, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 225, and the mRNA encoding the Cas protein is fully substituted with pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 165. Optionally, the first factor IX protein coding sequence comprises SEQ ID NO: 159.

In some such compositions, the albumin gene is a human albumin gene, wherein the composition comprises the guide RNA in the form of RNA, and the guide RNA comprises SEQ ID NO: 68 or 100, wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 226, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 166. Optionally, the first factor IX protein coding sequence comprises SEQ ID NO: 159.

In some such compositions, the albumin gene is a human albumin gene, wherein the composition comprises the guide RNA in the form of RNA, and the guide RNA comprises SEQ ID NO: 68 or 100, wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 226, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 165. Optionally, the first factor IX protein coding sequence comprises SEQ ID NO: 159.

In some such compositions, the albumin gene is a human albumin gene, wherein the composition comprises the guide RNA in the form of RNA, and the guide RNA comprises SEQ ID NO: 62 or 94, wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 226, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 166. Optionally, the first factor IX protein coding sequence comprises SEQ ID NO: 159.

In some such compositions, the albumin gene is a human albumin gene, wherein the composition comprises the guide RNA in the form of RNA, and the guide RNA comprises SEQ ID NO: 62 or 94, wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 226, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 165. Optionally, the first factor IX protein coding sequence comprises SEQ ID NO: 159.

In some such compositions, the albumin gene is a human albumin gene, wherein the composition comprises the guide RNA in the form of RNA, and the guide RNA comprises SEQ ID NO: 65 or 97, wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 226, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 166. Optionally, the first factor IX protein coding sequence comprises SEQ ID NO: 159.

In some such compositions, the albumin gene is a human albumin gene, wherein the composition comprises the guide RNA in the form of RNA, and the guide RNA comprises SEQ ID NO: 65 or 97, wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 226, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 165. Optionally, the first factor IX protein coding sequence comprises SEQ ID NO: 159.

In some such compositions, the albumin gene is a human albumin gene, wherein the composition comprises the guide RNA in the form of RNA, and the guide RNA comprises SEQ ID NO: 73 or 105, wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 226, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 166. Optionally, the first factor IX protein coding sequence comprises SEQ ID NO: 159.

In some such compositions, the albumin gene is a human albumin gene, wherein the composition comprises the guide RNA in the form of RNA, and the guide RNA comprises SEQ ID NO: 73 or 105, wherein the composition comprises the nucleic acid encoding the Cas protein, wherein the nucleic acid comprises an mRNA encoding the Cas protein, and the mRNA encoding the Cas protein comprises the sequence set forth in SEQ ID NO: 226, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 165. Optionally, the first factor IX protein coding sequence comprises SEQ ID NO: 159.

In some such compositions, the albumin gene is a human albumin gene, wherein the composition comprises the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine or N1-methyl-pseudouridine, optionally N1-methyl-pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the albumin gene is a human albumin gene, wherein the composition comprises the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 166. Optionally, the first factor IX protein coding sequence comprises SEQ ID NO: 159.

In some such compositions, the albumin gene is a human albumin gene, wherein the composition comprises the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine or N1-methyl-pseudouridine, optionally N1-methyl-pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the albumin gene is a human albumin gene, wherein the composition comprises the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 165. Optionally, the first factor IX protein coding sequence comprises SEQ ID NO: 159.

In some such compositions, the albumin gene is a human albumin gene, wherein the composition comprises the guide RNA in the form of RNA, the guide RNA comprises SEQ ID NO: 94, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine or N1-methyl-pseudouridine, optionally N1-methyl-pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the albumin gene is a human albumin gene, wherein the composition comprises the guide RNA in the form of RNA, the guide RNA comprises SEQ ID NO: 94, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 166. Optionally, the first factor IX protein coding sequence comprises SEQ ID NO: 159.

In some such compositions, the albumin gene is a human albumin gene, wherein the composition comprises the guide RNA in the form of RNA, the guide RNA comprises SEQ ID NO: 94, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine or N1-methyl-pseudouridine, optionally N1-methyl-pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the albumin gene is a human albumin gene, wherein the composition comprises the guide RNA in the form of RNA, the guide RNA comprises SEQ ID NO: 94, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 165. Optionally, the first factor IX protein coding sequence comprises SEQ ID NO: 159.

In some such compositions, the albumin gene is a human albumin gene, wherein the composition comprises the guide RNA in the form of RNA, the guide RNA comprises SEQ ID NO: 97, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine or N1-methyl-pseudouridine, optionally N1-methyl-pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the albumin gene is a human albumin gene, wherein the composition comprises the guide RNA in the form of RNA, the guide RNA comprises SEQ ID NO: 97, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 166. Optionally, the first factor IX protein coding sequence comprises SEQ ID NO: 159.

In some such compositions, the albumin gene is a human albumin gene, wherein the composition comprises the guide RNA in the form of RNA, the guide RNA comprises SEQ ID NO: 97, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine or N1-methyl-pseudouridine, optionally N1-methyl-pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the albumin gene is a human albumin gene, wherein the composition comprises the guide RNA in the form of RNA, the guide RNA comprises SEQ ID NO: 97, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 165. Optionally, the first factor IX protein coding sequence comprises SEQ ID NO: 159.

In some such compositions, the albumin gene is a human albumin gene, wherein the composition comprises the guide RNA in the form of RNA, the guide RNA comprises SEQ ID NO: 105, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine or N1-methyl-pseudouridine, optionally N1-methyl-pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the albumin gene is a human albumin gene, wherein the composition comprises the guide RNA in the form of RNA, the guide RNA comprises SEQ ID NO: 105, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 166, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 166. Optionally, the first factor IX protein coding sequence comprises SEQ ID NO: 159.

In some such compositions, the albumin gene is a human albumin gene, wherein the composition comprises the guide RNA in the form of RNA, the guide RNA comprises SEQ ID NO: 105, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine or N1-methyl-pseudouridine, optionally N1-methyl-pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the albumin gene is a human albumin gene, wherein the composition comprises the guide RNA in the form of RNA, the guide RNA comprises SEQ ID NO: 105, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition comprises the nucleic acid encoding the Cas protein, wherein the nucleic acid comprises an mRNA encoding the Cas protein, the mRNA encoding the Cas protein comprises the sequence set forth in SEQ ID NO: 226, and the mRNA encoding the Cas protein is fully substituted with pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such compositions, the nucleic acid construct is a bidirectional construct comprising the first factor IX protein coding sequence and a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 165; or (ii) the first factor IX protein coding sequence comprises SEQ ID NO: 165 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such compositions, the nucleic acid construct is a unidirectional construct comprising the first factor IX protein coding sequence, wherein the nucleic acid construct comprises from 5′ to 3′: a splice acceptor, the first factor IX protein coding sequence, and a polyadenylation signal, wherein the first factor IX protein coding sequence comprises SEQ ID NO: 159 or SEQ ID NO: 165, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. Optionally, the first factor IX protein coding sequence comprises SEQ ID NO: 165. Optionally, the first factor IX protein coding sequence comprises SEQ ID NO: 159.

In some such compositions, the composition is for use in a method of introducing a factor 9 nucleic acid into a cell, a method of integrating a factor 9 nucleic acid construct into a target gene in a cell, or a method of expressing factor IX in a cell. In some such compositions, the composition is for use in a method of treating a factor IX deficiency in a subject. In some such compositions, the composition is for use in a method of treating hemophilia B in a subject. In some such compositions, the cell is a neonatal cell. In some such compositions, the neonatal cell is from a human neonatal subject within 24 weeks after birth. In some such compositions, the neonatal cell is from a human neonatal subject within 12 weeks after birth. the neonatal cell is from a human neonatal subject within 8 weeks after birth. the neonatal cell is from a human neonatal subject within 4 weeks after birth. In some such compositions, the cell is not a neonatal cell. In some such compositions, the subject is a neonatal subject. In some such compositions, the neonatal subject is a human neonatal subject within 24 weeks after birth. In some such compositions, the neonatal subject is a human neonatal subject within 12 weeks after birth. In some such compositions, the neonatal subject is a human neonatal subject within 8 weeks after birth. In some such compositions, the neonatal subject is a human neonatal subject within 4 weeks after birth. In some such compositions, the subject is not a neonatal subject.

In another aspect, provided are cells comprising any of the above compositions. In some such cells, the nucleic acid construct is integrated into an endogenous target gene locus, and wherein factor IX protein is expressed from the endogenous target gene locus, or wherein the nucleic acid construct is integrated into intron 1 of an endogenous albumin locus, and wherein factor IX protein is expressed from the endogenous albumin locus. Some such cells are human cells. Some such cells are liver cells (e.g., human liver cells). Optionally, the liver cell is a hepatocyte. In some such cells, the cell is a neonatal cell. In some such cells, the neonatal cell is from a human neonatal subject within 24 weeks after birth. In some such cells, the neonatal cell is from a human neonatal subject within 12 weeks after birth. In some such cells, the neonatal cell is from a human neonatal subject within 8 weeks after birth. In some such cells, the neonatal cell is from a human neonatal subject within 4 weeks after birth. In some such cells, the cell is not a neonatal cell. In some such cells, the cell is ex vivo or in vitro. In some such cells, the cell is in vivo.

In another aspect, provided are methods of introducing a factor 9 nucleic acid into a cell, methods of integrating a factor 9 nucleic acid construct into a target gene in a cell, and methods of expressing factor IX in a cell. Some such methods are for introducing a factor 9 nucleic acid into a cell, comprising administering any of the above compositions to the cell. Some such methods are for integrating a factor 9 nucleic acid construct into a target gene in a cell, comprising administering any of the above compositions to the cell, wherein the nuclease agent cleaves the nuclease target site in the target gene to create a cleavage site, the nucleic acid construct is inserted into the cleavage site to create a modified target gene, and factor IX protein is expressed from the modified target gene. Some such methods are for expressing factor IX in a cell, comprising administering any of the above compositions to the cell, wherein the nuclease agent cleaves the nuclease target site in the target gene to create a cleavage site, the nucleic acid construct is inserted into the cleavage site to create a modified target gene, and factor IX protein is expressed from the modified target gene.

In some such methods, the nuclease agent comprises: (a) a Cas protein or a nucleic acid encoding the Cas protein; and (b) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

In some such methods, the nucleic acid construct, the Cas protein or the nucleic acid encoding the Cas protein, and the guide RNA or the one or more DNAs encoding the guide RNAs are administered simultaneously. In some such methods, the nucleic acid construct is not administered simultaneously with the Cas protein or the nucleic acid encoding the Cas protein and the guide RNA or the one or more DNAs encoding the guide RNAs.

In some such methods, the target gene is an albumin gene, and the nuclease target site is in intron 1 of the albumin gene. In some such methods, the cell is a liver cell. In some such methods, the cell is a hepatocyte. In some such methods, the cell is a human cell (e.g., a human liver cell). In some such methods, the cell is a neonatal cell. In some such methods, the neonatal cell is from a human neonatal subject within 24 weeks after birth. In some such methods, the neonatal cell is from a human neonatal subject within 12 weeks after birth. In some such methods, the neonatal cell is from a human neonatal subject within 8 weeks after birth. In some such methods, the neonatal cell is from a human neonatal subject within 4 weeks after birth. In some such methods, the cell is not a neonatal cell. In some such methods, the cell is in vivo. In some such methods, the cell is in vitro or ex vivo.

In another aspect, provided are methods of treating a factor IX deficiency in a subject, methods of treating hemophilia B in a subject, and methods of preventing or inhibiting spontaneous bleeding in a subject having hemophilia B. Some such methods are for treating a factor IX deficiency in a subject, comprising administering any of the above compositions to the subject. Some methods are for treating hemophilia B in a subject, comprising administering any of the above compositions to the subject. Some methods are for preventing or inhibiting spontaneous bleeding in a subject having hemophilia B, comprising administering any of the above compositions to the subject.

In some such methods, the hemophilia B is mild hemophilia B. In some such methods, the hemophilia B is moderate hemophilia B. In some such methods, the hemophilia B is severe hemophilia B.

In some such methods, the nuclease agent comprises: (a) a Cas protein or a nucleic acid encoding the Cas protein; and (b) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence. In some such methods, the guide RNA target sequence is in intron 1 of an albumin gene.

In some such methods, the nucleic acid construct, the Cas protein or the nucleic acid encoding the Cas protein, and the guide RNA or the one or more DNAs encoding the guide RNAs are administered simultaneously. In some such methods, the nucleic acid construct is not administered simultaneously with the Cas protein or the nucleic acid encoding the Cas protein and the guide RNA or the one or more DNAs encoding the guide RNAs.

In some such methods, the subject is a human subject. In some such methods, the subject is a neonatal subject. In some such methods, the neonatal subject is a human neonatal subject within 24 weeks after birth. In some such methods, the neonatal subject is a human neonatal subject within 12 weeks after birth. In some such methods, the neonatal subject is a human neonatal subject within 8 weeks after birth. In some such methods, the neonatal subject is a human neonatal subject within 4 weeks after birth. In some such methods, the subject is not a neonatal subject.

In some such methods, the method results in a therapeutically effective level of circulating factor IX protein in the subject. In some such methods, the method results in a therapeutically effective level of circulating factor IX coagulation activity in the subject. In some such methods, the method results in increased expression of the factor IX protein in the subject compared to a method comprising administering an episomal expression vector encoding the factor IX protein to a control subject. In some such methods, the method results in increased serum levels of the factor IX protein in the subject compared to a method comprising administering an episomal expression vector encoding the factor IX protein to a control subject. In some such methods, the method results in serum levels of the factor IX protein in the subject of at least about 1 μg/mL, at least about 2 μg/mL, at least about 3 μg/mL, at least about 4 μg/mL, or at least about 5 μg/mL. In some such methods, the method results in serum levels of the factor IX protein in the subject of at least about 5 μg/mL. In some such methods, the method results in serum levels of the factor IX protein in the subject of between about 1 μg/mL and about 10 μg/mL.

In some such methods, the method achieves circulatory factor IX protein or coagulation activity levels of at least about 1% of normal, at least about 5% of normal, at least about 10% of normal, at least about 15% of normal, at least about 20% of normal, at least about 25% of normal, at least about 30% of normal, at least about 35% of normal, at least about 40% of normal, at least about 45% of normal, or at least about 50% of normal. In some such methods, the method achieves circulatory factor IX protein or coagulation activity levels of between about 40% and about 150% or between about 40% and about 100% of normal.

In some such methods: (I) the subject has severe hemophilia, and the method achieves circulatory factor IX protein or coagulation activity levels of at least about 1% or more than about 1% of normal; (II) the subject has moderate hemophilia, and the method achieves circulatory factor IX protein or coagulation activity levels of at least about 5% or more than about 5% of normal; or (III) the subject has mild hemophilia, and the method achieves circulatory factor IX protein or coagulation activity levels of at least about 10%, at least about 15%, at least about 20%, at least about 25%, at least about 30%, at least about 35%, at least about 40%, at least about 45%, at least about 50%, more than about 10%, more than about 15%, more than about 20%, more than about 25%, more than about 30%, more than about 35%, more than about 40%, more than about 45%, or more than about 50% of normal.

In some such methods, the method achieves circulatory factor IX protein or coagulation activity levels of at least about 15% of normal.

In some such methods, the method achieves circulatory factor IX protein or coagulation activity levels of less than about 300%, less than about 250%, less than about 200%, or less than about 150% of normal.

In some such methods, the method increases circulatory factor IX protein or coagulation activity levels over the subject's baseline factor IX protein or coagulation activity levels by at least about 1%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or at least about 100%.

In some such methods, the circulatory factor IX protein or coagulation activity levels are sustained for at least about 1 month, at least about 2 months, at least about 3 months, at least about 6 months, at least about 1 year, or at least about 2 years after administering the composition. In some such methods, the expression or activity of the factor IX protein is at least 50% of the expression or activity of the factor IX protein at a peak level of expression measured for the human subject at six months after the administering. In some such methods, the expression or activity of the factor IX protein is at least 50% of the expression or activity of the factor IX protein at a peak level of expression measured for the human subject at one year after the administering. In some such methods, the expression or activity of the factor IX protein is at least 60% of the expression or activity of the factor IX protein at a peak level of expression measured for the human subject at six months after the administering. In some such methods, the expression or activity of the factor IX protein is at least 50% of the expression or activity of the factor IX protein at a peak level of expression measured for the human subject at two years after the administering. In some such methods, the expression or activity of the factor IX protein is at least 60% of the expression or activity of the factor IX protein at a peak level of expression measured for the human subject at two years after the administering. In some such methods, the expression or activity of the factor IX protein is at least 60% of the expression or activity of the factor IX protein at a peak level of expression measured for the human subject at six months after the administering.

In some such methods, the method further comprises assessing preexisting AAV immunity in the subject prior to administering the composition to the subject. In some such methods, the preexisting AAV immunity is preexisting AAV8 immunity. In some such methods, assessing preexisting AAV immunity comprises assessing immunogenicity using a total antibody immune assay or a neutralizing antibody assay.

Also provided are compositions and methods for inserting a nucleic acid encoding factor IX protein into a target genomic locus in a neonatal cell, a population of neonatal cells, or a neonatal subject or for expressing a nucleic acid encoding factor IX protein from a target genomic locus in a neonatal cell, a population of neonatal cells, or a neonatal subject. Also provided are methods of treating a factor IX deficiency, methods of preventing or inhibiting spontaneous bleeding, and methods of treating hemophilia B in a subject (e.g., neonatal subject. Also provided are neonatal cells or populations of neonatal cells comprising a nucleic acid encoding factor IX protein inserted into a target genomic locus.

In one aspect, provided are methods of inserting a nucleic acid encoding factor IX protein into a target genomic locus in a neonatal cell or a population of neonatal cells. Some such methods comprise administering to the neonatal cell or the population of neonatal cells: (a) a nucleic acid construct comprising a factor IX protein coding sequence; and (b) a nuclease agent or one or more nucleic acids encoding the nuclease agent, wherein the nuclease agent targets a nuclease target site in the target genomic locus, wherein the nuclease agent cleaves the nuclease target site, and the nucleic acid construct is inserted into the target genomic locus. In another aspect, provided are methods of expressing a factor IX protein from a target genomic locus in a neonatal cell or a population of neonatal cells. Some such methods comprise administering to the neonatal cell or the population of neonatal cells: (a) a nucleic acid construct comprising a factor IX protein coding sequence; and (b) a nuclease agent or one or more nucleic acids encoding the nuclease agent, wherein the nuclease agent targets a nuclease target site in the target genomic locus, wherein the nuclease agent cleaves the nuclease target site, the nucleic acid construct is inserted into the target genomic locus to create a modified target genomic locus, and the factor IX protein is expressed from the modified target genomic locus. In some such methods, the neonatal cell is a liver cell or the population of neonatal cells is a population of liver cells. In some such methods, the neonatal cell is a hepatocyte or the population of neonatal cells is a population of hepatocytes. In some such methods, the neonatal cell is a human cell or the population of neonatal cells is a population of human cells. In some such methods, the neonatal cell or the population of neonatal cells is from a neonatal subject within 24 weeks after birth. In some such methods, the neonatal cell or the population of neonatal cells is from a neonatal subject within 12 weeks after birth. In some such methods, the neonatal cell or the population of neonatal cells is from a neonatal subject within 8 weeks after birth. In some such methods, the neonatal cell or the population of neonatal cells is from a neonatal subject within 4 weeks after birth. In some such methods, the neonatal cell is in vitro or ex vivo or the population of neonatal cells is in vitro or ex vivo. In some such methods, the neonatal cell is in vivo in a neonatal subject or the population of neonatal cells is in vivo in a neonatal subject.

In another aspect, provided are methods of inserting a nucleic acid encoding a factor IX protein into a target genomic locus in a neonatal cell or a population of neonatal cells in a neonatal subject. Some such methods comprise administering to the neonatal subject: (a) a nucleic acid construct comprising a factor IX protein coding sequence; and (b) a nuclease agent or one or more nucleic acids encoding the nuclease agent, wherein the nuclease agent targets a nuclease target site in the target genomic locus, wherein the nuclease agent cleaves the nuclease target site, and the nucleic acid construct is inserted into the target genomic locus. In another aspect, provided are methods of expressing a factor IX protein from a target genomic locus in a neonatal cell or a population of neonatal cells in a neonatal subject. Some such methods comprise administering to the neonatal subject: (a) a nucleic acid construct comprising a factor IX protein coding sequence; and (b) a nuclease agent or one or more nucleic acids encoding the nuclease agent, wherein the nuclease agent targets a nuclease target site in the target genomic locus, wherein the nuclease agent cleaves the nuclease target site, the nucleic acid construct is inserted into the target genomic locus to create a modified target genomic locus, and the factor IX protein is expressed from the modified target genomic locus. In some such methods, the neonatal cell is a liver cell or the population of neonatal cells is a population of liver cells. In some such methods, the neonatal cell is a hepatocyte or the population of neonatal cells is a population of hepatocytes. In some such methods, the neonatal cell is a human cell or the population of neonatal cells is a population of human cells.

In another aspect, provided are methods of treating a factor IX deficiency in a neonatal subject in need thereof. Some such methods comprise administering to the neonatal subject: (a) a nucleic acid construct comprising a factor IX protein coding sequence; and (b) a nuclease agent or one or more nucleic acids encoding the nuclease agent, wherein the nuclease agent targets a nuclease target site in a target genomic locus, wherein the nuclease agent cleaves the nuclease target site, the nucleic acid construct is inserted into the target genomic locus to create a modified target genomic locus, and the factor IX protein is expressed from the modified target genomic locus. In another aspect, provided are methods of preventing or inhibiting spontaneous bleeding in a neonatal subject having hemophilia B. Some such methods comprise administering to the neonatal subject: (a) a nucleic acid construct comprising a factor IX protein coding sequence; and (b) a nuclease agent or one or more nucleic acids encoding the nuclease agent, wherein the nuclease agent targets a nuclease target site in a target genomic locus, wherein the nuclease agent cleaves the nuclease target site, the nucleic acid construct is inserted into the target genomic locus to create a modified target genomic locus, and the factor IX protein is expressed from the modified target genomic locus and prevents or inhibits spontaneous bleeding in the neonatal subject. In some such methods, the subject has hemophilia B. In another aspect, provided are methods of treating hemophilia B in a neonatal subject in need thereof. Some such methods comprise administering to the neonatal subject: (a) a nucleic acid construct comprising factor IX protein coding sequence; and (b) a nuclease agent or one or more nucleic acids encoding the nuclease agent, wherein the nuclease agent targets a nuclease target site in a target genomic locus, wherein the nuclease agent cleaves the nuclease target site, the nucleic acid construct is inserted into the target genomic locus to create a modified target genomic locus, and the factor IX protein is expressed from the modified target genomic locus, thereby treating the hemophilia B. In some such methods, the subject has mild hemophilia B. In some such methods, the subject has moderate hemophilia B. In some such methods, the subject has severe hemophilia B.

In some such methods, the neonatal subject is a human neonatal subject within 24 weeks after birth. In some such methods, the neonatal subject is a human neonatal subject within 12 weeks after birth. In some such methods, the neonatal subject is a human neonatal subject within 8 weeks after birth. In some such methods, the neonatal subject is a human neonatal subject within 4 weeks after birth.

In some such methods, the method results in increased expression of the factor IX protein in the subject compared to a method comprising administering an episomal expression vector encoding the factor IX protein to a control subject. In some such methods, the method results in increased serum levels of the factor IX protein in the subject compared to a method comprising administering an episomal expression vector encoding the factor IX protein to a control subject. In some such methods, the method results in serum levels of the factor IX protein in the subject of at least about 1 μg/mL, at least about 2 μg/mL, at least about 3 μg/mL, at least about 4 μg/mL, or at least about 5 μg/mL. In some such methods, the method results in serum levels of the factor IX protein in the subject of at least about 5 μg/mL. In some such methods, the method results in serum levels of the factor IX protein in the subject of between about 1 μg/mL and about 10 μg/mL. In some such methods, the method increases expression of factor IX protein over the subject's baseline expression of factor IX protein by at least about 1%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or at least about 100%. In some such methods, the method increases the level of factor IX protein in the serum over the subject's baseline serum level of factor IX protein by at least about 1%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or at least about 100%. In some such methods, the expression or activity of the factor IX protein is at least 50% of the expression or activity of the factor IX protein at a peak level of expression measured for the human subject at six months after the administering. In some such methods, the expression or activity of the factor IX protein is at least 50% of the expression or activity of the factor IX protein at a peak level of expression measured for the human subject at one year after the administering. In some such methods, the expression or activity of the factor IX protein is at least 60% of the expression or activity of the factor IX protein at a peak level of expression measured for the human subject at six months after the administering. In some such methods, the expression or activity of the factor IX protein is at least 50% of the expression or activity of the factor IX protein at a peak level of expression measured for the human subject at two years after the administering. In some such methods, the expression or activity of the factor IX protein is at least 60% of the expression or activity of the factor IX protein at a peak level of expression measured for the human subject at two years after the administering. In some such methods, the expression or activity of the factor IX protein is at least 60% of the expression or activity of the factor IX protein at a peak level of expression measured for the human subject at six months after the administering. In some such methods, the method results in a therapeutically effective level of circulating factor IX protein in the subject. In some such methods, the method results in a therapeutically effective level of circulating factor IX coagulation activity in the subject. In some such methods, the method achieves circulatory factor IX protein or coagulation activity levels of at least about 1% of normal, at least about 5% of normal, at least about 10% of normal, at least about 15% of normal, at least about 20% of normal, at least about 25% of normal, at least about 30% of normal, at least about 35% of normal, at least about 40% of normal, at least about 45% of normal, or at least about 50% of normal. In some such methods, the method achieves circulatory factor IX protein or coagulation activity levels of between about 40% and about 150% or between about 40% and about 100% of normal. In some such methods, the subject has severe hemophilia, and the method achieves circulatory factor IX protein or coagulation activity levels of at least about 1% or more than about 1% of normal. In some such methods, the subject has moderate hemophilia, and the method achieves circulatory factor IX protein or coagulation activity levels of at least about 5% or more than about 5% of normal. In some such methods, the subject has mild hemophilia, and the method achieves circulatory factor IX protein or coagulation activity levels of at least about 10%, at least about 15%, at least about 20%, at least about 25%, at least about 30%, at least about 35%, at least about 40%, at least about 45%, at least about 50%, more than about 10%, more than about 15%, more than about 20%, more than about 25%, more than about 30%, more than about 35%, more than about 40%, more than about 45%, or more than about 50% of normal. In some such methods, the method achieves circulatory factor IX protein or coagulation activity levels of at least about 15% of normal. In some such methods, the method achieves circulatory factor IX protein or coagulation activity levels of less than about 300%, less than about 250%, less than about 200%, or less than about 150% of normal. In some such methods, the method increases circulatory factor IX protein or coagulation activity levels over the subject's baseline factor IX protein or coagulation activity levels by at least about 1%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or at least about 100%. In some such methods, the subject is a human subject.

In some such methods, the nucleic acid construct is administered simultaneously with the nuclease agent or the one or more nucleic acids encoding the nuclease agent. In some such methods, the nucleic acid construct is not administered simultaneously with the nuclease agent or the one or more nucleic acids encoding the nuclease agent. In some such methods, the nucleic acid construct is administered prior to the nuclease agent or the one or more nucleic acids encoding the nuclease agent. In some such methods, the nucleic acid construct is administered after the nuclease agent or the one or more nucleic acids encoding the nuclease agent.

In some such methods, the factor IX protein coding sequence encodes a factor IX protein at least 99% identical to SEQ ID NO: 195, and optionally wherein the factor IX protein is not a hyperactive factor IX variant. In some such methods, the factor IX protein coding sequence encodes a factor IX protein comprising SEQ ID NO: 195. In some such methods, the factor IX protein coding sequence: (I) is at least 95% identical to any one of SEQ ID NOS: 166, 165, 164, and 167-171, optionally wherein the factor IX protein coding sequence is CpG-depleted and is codon-optimized, and optionally wherein the factor IX protein is not a hyperactive factor IX variant; or (II) is at least 99% identical to any one of SEQ ID NOS: 159, 160, and 161 and is: (i) CpG-depleted; (ii) modified to mutate one or more cryptic splice donor sequences; or (iii) CpG-depleted and modified to mutate one or more cryptic splice donor sequences, and optionally wherein the factor IX protein is not a hyperactive factor IX variant. In some such methods, the factor IX protein coding sequence: (I) is at least 99% identical to SEQ ID NO: 166, is CpG-depleted, and is codon-optimized, and optionally wherein the factor IX protein is not a hyperactive factor IX variant; (II) is at least 99% identical to SEQ ID NO: 165, is CpG-depleted, and is codon-optimized, and optionally wherein the factor IX protein is not a hyperactive factor IX variant; or (III) is at least 99% identical to SEQ ID NO: 159, is CpG-depleted, and is modified to mutate one or more cryptic splice donor sequences, and optionally wherein the factor IX protein is not a hyperactive factor IX variant. In some such methods, the factor IX protein coding sequence: (I) is at least 99% identical to SEQ ID NO: 166, is CpG-depleted, and is codon-optimized, and optionally wherein the factor IX protein is not a hyperactive factor IX variant; or (II) is at least 99% identical to SEQ ID NO: 159, is CpG-depleted, and is modified to mutate one or more cryptic splice donor sequences, and optionally wherein the factor IX protein is not a hyperactive factor IX variant. In some such methods, the factor IX protein coding sequence is at least 99% identical to SEQ ID NO: 166, is CpG-depleted, and is codon-optimized, and optionally wherein the factor IX protein is not a hyperactive factor IX variant. In some such methods, the factor IX protein coding sequence is at least 99% identical to SEQ ID NO: 159, is CpG-depleted, and is modified to mutate one or more cryptic splice donor sequences, and optionally wherein the factor IX protein is not a hyperactive factor IX variant. In some such methods, the factor IX protein coding sequence has all but one CpG dinucleotides removed or is fully CpG depleted. In some such methods, the factor IX protein coding sequence comprises SEQ ID NO: 166, 165, or 159. In some such methods, the factor IX protein coding sequence consists of SEQ ID NO: 166, 165, or 159. In some such methods, the factor IX protein coding sequence comprises SEQ ID NO: 166 or 159. In some such methods, the factor IX protein coding sequence consists of SEQ ID NO: 166 or 159. In some such methods, the factor IX protein coding sequence comprises SEQ ID NO: 166. In some such methods, the factor IX protein coding sequence consists of SEQ ID NO: 166. In some such methods, the factor IX protein coding sequence comprises SEQ ID NO: 159. In some such methods, the factor IX protein coding sequence consists of SEQ ID NO: 159.

In some such methods, the nucleic acid construct comprises a splice acceptor upstream of the factor IX protein coding sequence. In some such methods, the nucleic acid construct comprises a polyadenylation signal downstream of the factor IX protein coding sequence. In some such methods, the nucleic acid construct comprises a splice acceptor upstream of the factor IX protein coding sequence, and the nucleic acid construct comprises a polyadenylation signal downstream of the factor IX protein coding sequence. In some such methods, the nucleic acid construct does not comprise homology arms. In some such methods, the nucleic acid construct is inserted into the target genomic locus via non-homologous end joining. In some such methods, the nucleic acid construct comprises homology arms. In some such methods, the nucleic acid construct is inserted into the target genomic locus via homology-directed repair. In some such methods, the nucleic acid construct does not comprise a promoter that drives the expression of the factor IX protein.

In some such methods, the nucleic acid construct is a bidirectional construct, and the factor IX protein coding sequence is a first factor IX protein coding sequence. In some such methods, the nucleic acid construct comprises the first factor IX protein coding sequence and a reverse complement of a second factor IX protein coding sequence. In some such methods, the first factor IX protein coding sequence and the second factor IX protein coding sequence are different but encode the same factor IX protein sequence, and optionally wherein the factor IX protein is not a hyperactive factor IX variant. In some such methods, (i) the first factor IX protein coding sequence is at least 99% identical to SEQ ID NO: 159 and is: (i) CpG-depleted; (ii) modified to mutate one or more cryptic splice donor sequences; or (iii) CpG-depleted and modified to mutate one or more cryptic splice donor sequences, and optionally wherein the factor IX protein is not a hyperactive factor IX variant; and (ii) the second factor IX protein coding sequence is at least 95% identical to SEQ ID NO: 166, is CpG-depleted, and is codon-optimized, and optionally wherein the factor IX protein is not a hyperactive factor IX variant. In some such methods, (i) the first factor IX protein coding sequence is at least 95% identical to SEQ ID NO: 166, is CpG-depleted, and is codon-optimized, and optionally wherein the factor IX protein is not a hyperactive factor IX variant; and (ii) the second factor IX protein coding sequence is at least 99% identical to SEQ ID NO: 159 and is: (i) CpG-depleted; (ii) modified to mutate one or more cryptic splice donor sequences; or (iii) CpG-depleted and modified to mutate one or more cryptic splice donor sequences, and optionally wherein the factor IX protein is not a hyperactive factor IX variant. In some such methods, (i) the first factor IX protein coding sequence is at least 99% identical to SEQ ID NO: 159, is CpG-depleted, and is modified to mutate one or more cryptic splice donor sequences, and optionally wherein the factor IX protein is not a hyperactive factor IX variant; and (ii) the second factor IX protein coding sequence is at least 99% identical to SEQ ID NO: 166, is CpG-depleted, and is codon-optimized, and optionally wherein the factor IX protein is not a hyperactive factor IX variant. In some such methods, (i) the first factor IX protein coding sequence is at least 99% identical to SEQ ID NO: 166, is CpG-depleted, and is codon-optimized, and optionally wherein the factor IX protein is not a hyperactive factor IX variant; and (ii) the second factor IX protein coding sequence is at least 99% identical to SEQ ID NO: 159, is CpG-depleted, and is modified to mutate one or more cryptic splice donor sequences, and optionally wherein the factor IX protein is not a hyperactive factor IX variant. In some such methods, the first factor IX protein coding sequence has all but one CpG dinucleotides removed or is fully CpG depleted and the second factor IX protein coding sequence has all but one CpG dinucleotides removed or is fully CpG depleted. In some such methods, the first factor IX protein coding sequence encodes a factor IX protein at least 99% identical to SEQ ID NO: 195 and optionally wherein the factor IX protein is not a hyperactive factor IX variant, and the second factor IX protein coding sequence encodes a factor IX protein at least 99% identical to SEQ ID NO: 195 and optionally wherein the factor IX protein is not a hyperactive factor IX variant. In some such methods, the first factor IX protein coding sequence encodes a factor IX protein comprising SEQ ID NO: 195, and the second factor IX protein coding sequence encodes a factor IX protein comprising SEQ ID NO: 195. In some such methods, the first factor IX protein coding sequence comprises SEQ ID NO: 159 and the second factor IX protein coding sequence comprises SEQ ID NO: 166. In some such methods, the second factor IX protein coding sequence comprises SEQ ID NO: 166 and the second factor IX protein coding sequence comprises SEQ ID NO: 159. In some such methods, the first factor IX protein coding sequence consists of SEQ ID NO: 159 and the second factor IX protein coding sequence consists of SEQ ID NO: 166. In some such methods, the first factor IX protein coding sequence consists of SEQ ID NO: 166 and the second factor IX protein coding sequence consists of SEQ ID NO: 159. In some such methods, the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor. In some such methods, the first factor IX protein coding sequence and the second factor IX protein coding sequence are different but encode the same factor IX protein sequence, and wherein the first polyadenylation signal and the second polyadenylation signal are different.

In some such methods, the nucleic acid construct is a bidirectional construct, wherein the factor IX protein coding sequence is a first factor IX protein coding sequence, and the bidirectional construct further comprises a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such methods, the nucleic acid construct comprises SEQ ID NO: 210 or 180 or the reverse complement thereof.

In some such methods, the nucleic acid construct is single-stranded DNA or double-stranded DNA. In some such methods, the nucleic acid construct is single-stranded DNA.

In some such methods, the nucleic acid construct is in a nucleic acid vector or a lipid nanoparticle. In some such methods, the nucleic acid construct is in the nucleic acid vector. In some such methods, the nucleic acid vector is a viral vector. In some such methods, the nucleic acid vector is an adeno-associated viral (AAV) vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such methods, the AAV vector is a single-stranded AAV (ssAAV) vector. In some such methods, the AAV vector is derived from an AAV8 vector, an AAV3B vector, an AAV5 vector, an AAV6 vector, an AAV7 vector, an AAV9 vector, an AAVrh.74 vector, or an AAVhu.37 vector. In some such methods, the AAV vector is a recombinant AAV8 (rAAV8) vector. In some such methods, the AAV vector is a single-stranded rAAV8 vector.

In some such methods, the nucleic acid construct is a bidirectional construct, wherein the factor IX protein coding sequence is a first factor IX protein coding sequence, and the bidirectional construct further comprises a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196.

In some such methods, the nucleic acid construct is a unidirectional construct. In some such methods, the nucleic acid construct is a unidirectional construct comprising the factor IX protein coding sequence, wherein the nucleic acid construct comprises from 5′ to 3′: a splice acceptor, the factor IX protein coding sequence, and a polyadenylation signal, wherein the factor IX protein coding sequence comprises SEQ ID NO: 159 or SEQ ID NO: 166; wherein the nucleic acid construct does not comprise a promoter that drives the expression of the factor IX protein, and wherein the nucleic acid construct does not comprise homology arms. In some such methods, the factor IX protein coding sequence comprises SEQ ID NO: 166. In some such methods, the factor IX protein coding sequence comprises SEQ ID NO: 159.

In some such methods, the nucleic acid construct is single-stranded DNA or double-stranded DNA. In some such methods, the nucleic acid construct is single-stranded DNA. In some such methods, the nucleic acid construct is in a nucleic acid vector or a lipid nanoparticle. In some such methods, the nucleic acid construct is in the nucleic acid vector. In some such methods, the nucleic acid vector is a viral vector. In some such methods, the nucleic acid vector is an adeno-associated viral (AAV) vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such methods, the AAV vector is a single-stranded AAV (ssAAV) vector. In some such methods, the AAV vector is derived from an AAV8 vector, an AAV3B vector, an AAV5 vector, an AAV6 vector, an AAV7 vector, an AAV9 vector, an AAVrh.74 vector, or an AAVhu.37 vector. In some such methods, the AAV vector is a recombinant AAV8 (rAAV8) vector. In some such methods, the AAV vector is a single-stranded rAAV8 vector.

In some such methods, the nucleic acid construct is a unidirectional construct comprising the factor IX protein coding sequence, wherein the nucleic acid construct comprises from 5′ to 3′: a splice acceptor, the factor IX protein coding sequence, and a polyadenylation signal, wherein the factor IX protein coding sequence comprises SEQ ID NO: 159 or SEQ ID NO: 166, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such methods, the factor IX protein coding sequence comprises SEQ ID NO: 166. In some such methods, the factor IX protein coding sequence comprises SEQ ID NO: 159.

In some such methods, the target genomic locus is an albumin gene, optionally wherein the albumin gene is a human albumin gene. In some such methods, the nuclease target site is in intron 1 of the albumin gene. In some such methods, the nuclease agent comprises: (a) a zinc finger nuclease (ZFN); (b) a transcription activator-like effector nuclease (TALEN); or (c) (i) a Cas protein or a nucleic acid encoding the Cas protein; and (ii) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

In some such methods, the nuclease agent comprises: (a) a Cas protein or a nucleic acid encoding the Cas protein; and (b) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence. In some such methods, the guide RNA target sequence is in intron 1 of an albumin gene. In some such methods, the albumin gene is a human albumin gene. In some such methods, the DNA-targeting segment comprises at least 17, at least 18, at least 19, or at least 20 contiguous nucleotides of the sequence set forth in any one of SEQ ID NOS: 30-61, optionally wherein the DNA-targeting segment comprises at least 17, at least 18, at least 19, or at least 20 contiguous nucleotides of the sequence set forth in any one of SEQ ID NOS: 36, 30, 33, and 41. In some such methods, the DNA-targeting segment is at least 90% or at least 95% identical to the sequence set forth in any one of SEQ ID NOS: 30-61, optionally wherein the DNA-targeting segment is at least 90% or at least 95% identical to the sequence set forth in any one of SEQ ID NOS: 36, 30, 33, and 41. In some such methods, the DNA-targeting segment comprises any one of SEQ ID NOS: 30-61, optionally wherein the DNA-targeting segment comprises any one of SEQ ID NOS: 36, 30, 33, and 41. In some such methods, the DNA-targeting segment consists of any one of SEQ ID NOS: 30-61, optionally wherein the DNA-targeting segment consists of any one of SEQ ID NOS: 36, 30, 33, and 41. In some such methods, the guide RNA comprises any one of SEQ ID NOS: 62-125, optionally wherein the guide RNA comprises any one of SEQ ID NOS: 68, 100, 62, 94, 65, 97, 73, and 105. In some such methods, the DNA-targeting segment comprises at least 17, at least 18, at least 19, or at least 20 contiguous nucleotides of SEQ ID NO: 36. In some such methods, the DNA-targeting segment is at least 90% or at least 95% identical to SEQ ID NO: 36. In some such methods, the DNA-targeting segment comprises SEQ ID NO: 36. In some such methods, the DNA-targeting segment consists of SEQ ID NO: 36. In some such methods, the guide RNA comprises SEQ ID NO: 68 or 100.

In some such methods, the method comprises administering the guide RNA in the form of RNA. In some such methods, the guide RNA comprises at least one modification. In some such methods, the at least one modification comprises a 2′-O-methyl-modified nucleotide. In some such methods, the at least one modification comprises a phosphorothioate bond between nucleotides. In some such methods, the at least one modification comprises a modification at one or more of the first five nucleotides at the 5′ end of the guide RNA. In some such methods, the at least one modification comprises a modification at one or more of the last five nucleotides at the 3′ end of the guide RNA. In some such methods, the at least one modification comprises phosphorothioate bonds between the first four nucleotides at the 5′ end of the guide RNA. In some such methods, the at least one modification comprises phosphorothioate bonds between the last four nucleotides at the 3′ end of the guide RNA. In some such methods, the at least one modification comprises 2′-O-methyl-modified nucleotides at the first three nucleotides at the 5′ end of the guide RNA. In some such methods, the at least one modification comprises 2′-O-methyl-modified nucleotides at the last three nucleotides at the 3′ end of the guide RNA. In some such methods, the at least one modification comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA. In some such methods, the guide RNA is a single guide RNA (sgRNA). In some such methods, the method comprises administering the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

In some such methods, the Cas protein is a Cas9 protein. In some such methods, the Cas9 protein is derived from a Streptococcus pyogenes Cas9 protein, a Staphylococcus aureus Cas9 protein, a Campylobacter jejuni Cas9 protein, a Streptococcus thermophilus Cas9 protein, or a Neisseria meningitidis Cas9 protein. In some such methods, the Cas protein is derived from a Streptococcus pyogenes Cas9 protein. In some such methods, the Cas protein comprises the sequence set forth in SEQ ID NO: 11. In some such methods, the nucleic acid encoding the Cas protein is codon-optimized for expression in a mammalian cell or a human cell. In some such methods, the method comprises administering the nucleic acid encoding the Cas protein, wherein the nucleic acid comprises an mRNA encoding the Cas protein. In some such methods, the mRNA encoding the Cas protein comprises at least one modification. In some such methods, the mRNA encoding the Cas protein is modified to comprise a modified uridine at one or more or all uridine positions. In some such methods, the modified uridine is pseudouridine or N1-methyl-pseudouridine, optionally N1-methyl-pseudouridine. In some such methods, the mRNA encoding the Cas protein is fully substituted with pseudouridine or N1-methyl-pseudouridine, optionally N1-methyl-pseudouridine. In some such methods, the mRNA encoding the Cas protein is modified to comprise a modified uridine at one or more or all uridine positions. In some such methods, the modified uridine is pseudouridine. In some such methods, the mRNA encoding the Cas protein is fully substituted with pseudouridine. In some such methods, the mRNA encoding the Cas protein comprises a 5′ cap. In some such methods, the mRNA encoding the Cas protein comprises a poly(A) tail. In some such methods, the mRNA encoding the Cas protein comprises the sequence set forth in SEQ ID NO: 226, 225, or 12. In some such methods, the method comprises administering the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine or N1-methyl-pseudouridine, optionally N1-methyl-pseudouridine, comprises a 5′ cap, and comprises a poly(A) tail. In some such methods, the method comprises administering the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine, comprises a 5′ cap, and comprises a poly(A) tail.

In some such methods, the method comprises administering the guide RNA in the form of RNA, and the guide RNA comprises SEQ ID NO: 68 or 100, and wherein the method comprises administering the nucleic acid encoding the Cas protein, wherein the nucleic acid comprises an mRNA encoding the Cas protein, and the mRNA encoding the Cas protein comprises the sequence set forth in SEQ ID NO: 226, 225, or 12. In some such methods, the method comprises administering the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the method comprises administering the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine or N1-methyl-pseudouridine, optionally N1-methyl-pseudouridine, comprises a 5′ cap, and comprises a poly(A) tail. In some such methods, the method comprises administering the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and wherein the method comprises administering the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine, comprises a 5′ cap, and comprises a poly(A) tail. In some such methods, the nucleic acid construct is a bidirectional construct, wherein the factor IX protein coding sequence is a first factor IX protein coding sequence, and wherein the bidirectional construct further comprises a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such methods, the nucleic acid construct is a unidirectional construct comprising the factor IX protein coding sequence, wherein the nucleic acid construct comprises from 5′ to 3′: a splice acceptor, the factor IX protein coding sequence, and a polyadenylation signal, wherein the factor IX protein coding sequence comprises SEQ ID NO: 159 or SEQ ID NO: 166, wherein the nucleic acid construct does not comprise a promoter that drives the expression of the factor IX protein, and wherein the nucleic acid construct does not comprise homology arms. In some such methods, the factor IX protein coding sequence comprises SEQ ID NO: 166. In some such methods, the factor IX protein coding sequence comprises SEQ ID NO: 159. In some such methods, the nucleic acid construct is a bidirectional construct, wherein the factor IX protein coding sequence is a first factor IX protein coding sequence, and wherein the bidirectional construct further comprises a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the nucleic acid construct does not comprise homology arms. In some such methods, the nucleic acid construct is a unidirectional construct comprising the factor IX protein coding sequence, wherein the nucleic acid construct comprises from 5′ to 3′: a splice acceptor, the factor IX protein coding sequence, and a polyadenylation signal, wherein the factor IX protein coding sequence comprises SEQ ID NO: 159 or SEQ ID NO: 166, wherein the nucleic acid construct does not comprise a promoter that drives the expression of the factor IX protein, and wherein the nucleic acid construct does not comprise homology arms. In some such methods, the factor IX protein coding sequence comprises SEQ ID NO: 166. In some such methods, the factor IX protein coding sequence comprises SEQ ID NO: 159.

In some such methods, the Cas protein or the nucleic acid encoding the Cas protein and the guide RNA or the one or more DNAs encoding the guide RNA are associated with a lipid nanoparticle. In some such methods, the lipid nanoparticle comprises a cationic lipid, a neutral lipid, a helper lipid, and a stealth lipid. In some such methods, the cationic lipid is Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate). In some such methods, the neutral lipid is di stearoylphosphatidylcholine or 1,2-distearoyl-sn-glycero-3-phosphocholine (DSPC). In some such methods, the helper lipid is cholesterol. In some such methods, the stealth lipid is PEG2k-DMG. In some such methods, the cationic lipid is Lipid A, the neutral lipid is DSPC, the helper lipid is cholesterol, and the stealth lipid is PEG2k-DMG. In some such methods, the lipid nanoparticle comprises four lipids at the following molar ratios: about 50 mol % Lipid A, about 9 mol % DSPC, about 38 mol % cholesterol, and about 3 mol % PEG2k-DMG.

In some such methods, the albumin gene is a human albumin gene, wherein the method comprises administering the guide RNA in the form of RNA, and the guide RNA comprises SEQ ID NO: 68 or 100, wherein the method comprises administering the nucleic acid encoding the Cas protein, wherein the nucleic acid comprises an mRNA encoding the Cas protein, and the mRNA encoding the Cas protein comprises the sequence set forth in SEQ ID NO: 226, 225, or 12, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such methods, the albumin gene is a human albumin gene, wherein the method comprises administering the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the method comprises administering the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine or N1-methyl-pseudouridine, optionally N1-methyl-pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such methods, the albumin gene is a human albumin gene, wherein the method comprises administering the guide RNA in the form of RNA, the guide RNA comprises SEQ ID NO: 100, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the method comprises administering the nucleic acid encoding the Cas protein, wherein the nucleic acid comprises an mRNA encoding the Cas protein, the mRNA encoding the Cas protein comprises the sequence set forth in SEQ ID NO: 226, 225, or 12, and the mRNA encoding the Cas protein is fully substituted with pseudouridine, comprises a 5′ cap, and comprises a poly(A) tail, and wherein the guide RNA and the mRNA encoding the Cas protein are associated with a lipid nanoparticle comprising Lipid A, DSPC, cholesterol, and PEG2k-DMG, optionally at the following molar ratios: about 50 mol % Lipid A, about 9 mol % DSPC, about 38 mol % cholesterol, and about 3 mol % PEG2k-DMG. In some such methods, the nucleic acid construct is a bidirectional construct, wherein the factor IX protein coding sequence is a first factor IX protein coding sequence, and wherein the bidirectional construct further comprises a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such methods, the nucleic acid construct is a unidirectional construct comprising the factor IX protein coding sequence, wherein the nucleic acid construct comprises from 5′ to 3′: a splice acceptor, the factor IX protein coding sequence, and a polyadenylation signal, wherein the factor IX protein coding sequence comprises SEQ ID NO: 159 or SEQ ID NO: 166, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such methods, the factor IX protein coding sequence comprises SEQ ID NO: 166. In some such methods, the factor IX protein coding sequence comprises SEQ ID NO: 159. In some such methods, the nucleic acid construct is a bidirectional construct, wherein the factor IX protein coding sequence is a first factor IX protein coding sequence, and wherein the bidirectional construct further comprises a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such methods, the nucleic acid construct is a unidirectional construct comprising the factor IX protein coding sequence, wherein the nucleic acid construct comprises from 5′ to 3′: a splice acceptor, the factor IX protein coding sequence, and a polyadenylation signal, wherein the factor IX protein coding sequence comprises SEQ ID NO: 159 or SEQ ID NO: 166, wherein the nucleic acid construct does not comprise a promoter that drives the expression of the factor IX protein, wherein the nucleic acid construct does not comprise homology arms, and wherein the nucleic acid construct is in a single-stranded rAAV8 vector, optionally wherein the nucleic acid construct is flanked by inverted terminal repeats (ITRs) on each end, optionally wherein the ITR on at least one end comprises, consists essentially of, or consists of SEQ ID NO: 198, and optionally wherein the ITR on each end comprises, consists essentially of, or consists of SEQ ID NO: 198, or optionally wherein the ITR on at least one end comprises, consists essentially of, or consists of SEQ ID NO: 196, and optionally wherein the ITR on each end comprises, consists essentially of, or consists of SEQ ID NO: 196. In some such methods, the factor IX protein coding sequence comprises SEQ ID NO: 166. In some such methods, the factor IX protein coding sequence comprises SEQ ID NO: 159.

In some such methods, the method further comprises assessing preexisting AAV immunity in the subject prior to administering the composition to the subject. In some such methods, the preexisting AAV immunity is preexisting AAV8 immunity. In some such methods, assessing preexisting AAV immunity comprises assessing immunogenicity using a total antibody immune assay or a neutralizing antibody assay.

In another aspect, provided is a neonatal cell or a population of neonatal cells made by any of the above methods. In another aspect, provided is a neonatal cell or a population of neonatal cells comprising a nucleic acid construct comprising a factor IX protein coding sequence inserted into a target genomic locus. In some such neonatal cells or populations of neonatal cells, the neonatal cell is a liver cell or the population of neonatal cells is a population of liver cells. In some such neonatal cells or populations of neonatal cells, the neonatal cell is a hepatocyte or the population of neonatal cells is a population of hepatocytes. In some such neonatal cells or populations of neonatal cells, the neonatal cell is a human cell or the population of neonatal cells is a population of human cells. In some such neonatal cells or populations of neonatal cells, the neonatal cell or the population of neonatal cells is from a human neonatal subject within 24 weeks after birth. In some such neonatal cells or populations of neonatal cells, the neonatal cell or the population of neonatal cells is from a human neonatal subject within 12 weeks after birth. In some such neonatal cells or populations of neonatal cells, the neonatal cell or the population of neonatal cells is from a human neonatal subject within 8 weeks after birth. In some such neonatal cells or populations of neonatal cells, the neonatal cell or the population of neonatal cells is from a human neonatal subject within 4 weeks after birth. In some such neonatal cells or populations of neonatal cells, the neonatal cell is in vitro or ex vivo or the population of neonatal cells is in vitro or ex vivo. In some such neonatal cells or populations of neonatal cells, the neonatal cell is in vivo in a subject or the population of neonatal cells is in vivo. In some such neonatal cells or populations of neonatal cells, the factor IX protein is expressed.

In some such neonatal cells or populations of neonatal cells, the factor IX protein coding sequence encodes a factor IX protein at least 99% identical to SEQ ID NO: 195, and optionally wherein the factor IX protein is not a hyperactive factor IX variant. In some such neonatal cells or populations of neonatal cells, the factor IX protein coding sequence encodes a factor IX protein comprising SEQ ID NO: 195. In some such neonatal cells or populations of neonatal cells, the factor IX protein coding sequence: (I) is at least 95% identical to any one of SEQ ID NOS: 166, 165, 164, and 167-171, optionally wherein the factor IX protein coding sequence is CpG-depleted and is codon-optimized, and optionally wherein the factor IX protein is not a hyperactive factor IX variant; or (II) is at least 99% identical to any one of SEQ ID NOS: 159, 160, and 161 and is: (i) CpG-depleted; (ii) modified to mutate one or more cryptic splice donor sequences; or (iii) CpG-depleted and modified to mutate one or more cryptic splice donor sequences, and optionally wherein the factor IX protein is not a hyperactive factor IX variant. In some such neonatal cells or populations of neonatal cells, the factor IX protein coding sequence: (I) is at least 99% identical to SEQ ID NO: 166, is CpG-depleted, and is codon-optimized, and optionally wherein the factor IX protein is not a hyperactive factor IX variant; (II) is at least 99% identical to SEQ ID NO: 165, is CpG-depleted, and is codon-optimized, and optionally wherein the factor IX protein is not a hyperactive factor IX variant; or (III) is at least 99% identical to SEQ ID NO: 159, is CpG-depleted, and is modified to mutate one or more cryptic splice donor sequences, and optionally wherein the factor IX protein is not a hyperactive factor IX variant. In some such neonatal cells or populations of neonatal cells, the factor IX protein coding sequence: (I) is at least 99% identical to SEQ ID NO: 166, is CpG-depleted, and is codon-optimized, and optionally wherein the factor IX protein is not a hyperactive factor IX variant; or (II) is at least 99% identical to SEQ ID NO: 159, is CpG-depleted, and is modified to mutate one or more cryptic splice donor sequences, and optionally wherein the factor IX protein is not a hyperactive factor IX variant. In some such neonatal cells or populations of neonatal cells, the factor IX protein coding sequence is at least 99% identical to SEQ ID NO: 166, is CpG-depleted, and is codon-optimized, and optionally wherein the factor IX protein is not a hyperactive factor IX variant. In some such neonatal cells or populations of neonatal cells, the factor IX protein coding sequence is at least 99% identical to SEQ ID NO: 159, is CpG-depleted, and is modified to mutate one or more cryptic splice donor sequences, and optionally wherein the factor IX protein is not a hyperactive factor IX variant. In some such neonatal cells or populations of neonatal cells, the factor IX protein coding sequence has all but one CpG dinucleotides removed or is fully CpG depleted. In some such neonatal cells or populations of neonatal cells, the factor IX protein coding sequence comprises SEQ ID NO: 166, 165, or 159. In some such neonatal cells or populations of neonatal cells, the factor IX protein coding sequence consists of SEQ ID NO: 166, 165, or 159. In some such neonatal cells or populations of neonatal cells, the factor IX protein coding sequence comprises SEQ ID NO: 166 or 159. In some such neonatal cells or populations of neonatal cells, the factor IX protein coding sequence consists of SEQ ID NO: 166 or 159. In some such neonatal cells or populations of neonatal cells, the factor IX protein coding sequence comprises SEQ ID NO: 166. In some such neonatal cells or populations of neonatal cells, the factor IX protein coding sequence consists of SEQ ID NO: 166. In some such neonatal cells or populations of neonatal cells, the factor IX protein coding sequence comprises SEQ ID NO: 159. In some such neonatal cells or populations of neonatal cells, the factor IX protein coding sequence consists of SEQ ID NO: 159.

In some such neonatal cells or populations of neonatal cells, the nucleic acid construct comprises a splice acceptor upstream of the factor IX protein coding sequence. In some such neonatal cells or populations of neonatal cells, the nucleic acid construct comprises a polyadenylation signal downstream of the factor IX protein coding sequence. In some such neonatal cells or populations of neonatal cells, the nucleic acid construct comprises a splice acceptor upstream of the factor IX protein coding sequence, and the nucleic acid construct comprises a polyadenylation signal downstream of the factor IX protein coding sequence. In some such neonatal cells or populations of neonatal cells, the nucleic acid construct does not comprise a promoter that drives the expression of the factor IX protein, and wherein the factor IX protein coding sequence is operably linked to an endogenous promoter at the target genomic locus.

In some such neonatal cells or populations of neonatal cells, the nucleic acid construct is a bidirectional construct, and the factor IX protein coding sequence is a first factor IX protein coding sequence. In some such neonatal cells or populations of neonatal cells, the nucleic acid construct comprises the first factor IX protein coding sequence and a reverse complement of a second factor IX protein coding sequence. In some such neonatal cells or populations of neonatal cells, the first factor IX protein coding sequence and the second factor IX protein coding sequence are different but encode the same factor IX protein sequence, and optionally wherein the factor IX protein is not a hyperactive factor IX variant. In some such neonatal cells or populations of neonatal cells, (i) the first factor IX protein coding sequence is at least 99% identical to SEQ ID NO: 159 and is: (i) CpG-depleted; (ii) modified to mutate one or more cryptic splice donor sequences; or (iii) CpG-depleted and modified to mutate one or more cryptic splice donor sequences, and optionally wherein the factor IX protein is not a hyperactive factor IX variant; and (ii) the second factor IX protein coding sequence is at least 95% identical to SEQ ID NO: 166, is CpG-depleted, and is codon-optimized, and optionally wherein the factor IX protein is not a hyperactive factor IX variant. In some such neonatal cells or populations of neonatal cells, (i) the first factor IX protein coding sequence is at least 95% identical to SEQ ID NO: 166, is CpG-depleted, and is codon-optimized, and optionally wherein the factor IX protein is not a hyperactive factor IX variant; and (ii) the second factor IX protein coding sequence is at least 99% identical to SEQ ID NO: 159 and is: (i) CpG-depleted; (ii) modified to mutate one or more cryptic splice donor sequences; or (iii) CpG-depleted and modified to mutate one or more cryptic splice donor sequences, and optionally wherein the factor IX protein is not a hyperactive factor IX variant. In some such neonatal cells or populations of neonatal cells, (i) the first factor IX protein coding sequence is at least 99% identical to SEQ ID NO: 159, is CpG-depleted, and is modified to mutate one or more cryptic splice donor sequences, and optionally wherein the factor IX protein is not a hyperactive factor IX variant; and (ii) the second factor IX protein coding sequence is at least 99% identical to SEQ ID NO: 166, is CpG-depleted, and is codon-optimized, and optionally wherein the factor IX protein is not a hyperactive factor IX variant. In some such neonatal cells or populations of neonatal cells, (i) the first factor IX protein coding sequence is at least 99% identical to SEQ ID NO: 166, is CpG-depleted, and is codon-optimized, and optionally wherein the factor IX protein is not a hyperactive factor IX variant; and (ii) the second factor IX protein coding sequence is at least 99% identical to SEQ ID NO: 159, is CpG-depleted, and is modified to mutate one or more cryptic splice donor sequences, and optionally wherein the factor IX protein is not a hyperactive factor IX variant. In some such neonatal cells or populations of neonatal cells, the first factor IX protein coding sequence has all but one CpG dinucleotides removed or is fully CpG depleted and the second factor IX protein coding sequence has all but one CpG dinucleotides removed or is fully CpG depleted. In some such neonatal cells or populations of neonatal cells, the first factor IX protein coding sequence encodes a factor IX protein at least 99% identical to SEQ ID NO: 195 and optionally wherein the factor IX protein is not a hyperactive factor IX variant, and the second factor IX protein coding sequence encodes a factor IX protein at least 99% identical to SEQ ID NO: 195 and optionally wherein the factor IX protein is not a hyperactive factor IX variant. In some such neonatal cells or populations of neonatal cells, the first factor IX protein coding sequence encodes a factor IX protein comprising SEQ ID NO: 195, and the second factor IX protein coding sequence encodes a factor IX protein comprising SEQ ID NO: 195. In some such neonatal cells or populations of neonatal cells, the first factor IX protein coding sequence comprises SEQ ID NO: 159 and the second factor IX protein coding sequence comprises SEQ ID NO: 166. In some such neonatal cells or populations of neonatal cells, the second factor IX protein coding sequence comprises SEQ ID NO: 166 and the second factor IX protein coding sequence comprises SEQ ID NO: 159. In some such neonatal cells or populations of neonatal cells, the first factor IX protein coding sequence consists of SEQ ID NO: 159 and the second factor IX protein coding sequence consists of SEQ ID NO: 166. In some such neonatal cells or populations of neonatal cells, the first factor IX protein coding sequence consists of SEQ ID NO: 166 and the second factor IX protein coding sequence consists of SEQ ID NO: 159. In some such neonatal cells or populations of neonatal cells, the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor. In some such neonatal cells or populations of neonatal cells, the first factor IX protein coding sequence and the second factor IX protein coding sequence are different but encode the same factor IX protein sequence, and wherein the first polyadenylation signal and the second polyadenylation signal are different.

In some such neonatal cells or populations of neonatal cells, the nucleic acid construct is a bidirectional construct, wherein the factor IX protein coding sequence is a first factor IX protein coding sequence, and the bidirectional construct further comprises a reverse complement of a second factor IX protein coding sequence, wherein the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 159 and the second factor IX protein coding sequence comprises SEQ ID NO: 166; or (ii) the first factor IX protein coding sequence comprises SEQ ID NO: 166 and the second factor IX protein coding sequence comprises SEQ ID NO: 159, wherein the nucleic acid construct does not comprise a promoter that drives the expression of the factor IX protein, and wherein the factor IX protein coding sequence is operably linked to an endogenous promoter at the target genomic locus. In some such neonatal cells or populations of neonatal cells, the nucleic acid construct comprises SEQ ID NO: 210 or 180 or the reverse complement thereof.

In some such neonatal cells or populations of neonatal cells, the target genomic locus is an albumin gene, optionally wherein the albumin gene is a human albumin gene. In some such neonatal cells or populations of neonatal cells, the nuclease target site is in intron 1 of the albumin gene.

### BRIEF DESCRIPTION OF THE FIGURES

FIG. 1 shows a schematic of the coagulation cascade.

FIG. 2 shows a schematic for CRISPR/Cas9-mediated insertion of a bidirectional AAV insertion template at the albumin (ALB) locus. The human ALB locus is depicted, with the Cas9 cut site denoted with scissors. Splice acceptor sites flanking factor 9 (F9) transgenes in the F9 DNA insertion template are depicted. Polyadenylation sequences to terminate transcription following each F9 transgene are depicted. Following insertion and transcription driven by the endogenous ALB promoter, splicing between ALB exon 1 and the inserted F9 DNA template occurs, diagrammed in dashed lines, to produce a hybrid ALB-F9 mRNA. The ALB signal peptide (sp; encoded in exon 1 and diagrammed in brackets) promotes secretion of factor IX (FIX) and is removed, along with the FIX propeptide, during protein maturation to yield mature wild type FIX in plasma.

FIG. 3 shows development candidate LNPF9, which is a lipid nanoparticle containing Cas9 mRNA and sgRNA 9860 targeting human ALB intron 1, and REGV131, which is a recombinant AAV8 (rAAV8) capsid packaged with a bidirectional F9 insertion template encoded by Insert 30.

FIG. 4 shows a schematic for the bidirectional F9 insertion template encoded by Insert 30.

FIG. 5 shows in vitro FIX expression in PHH. PHH were seeded in a 96-well plate and then treated with a constant 6.0E+4 vg/cell of the indicated insertion template (Insert 72, Insert 29, or Insert 30) packaged into rAAV8. LNP comprising Cas9 mRNA and one of four sgRNA targeting ALB intron 1 (g9860, g9857, g9874, and g9844) was added to replicate wells at concentrations ranging from 1000 ng/mL to 4.1 ng/ML across a 3-fold serial dilution series. Cells were incubated at 37° C., with media changes at day 3 and day 5, and hFIX expression was measured on day 8 via hFIX ELISA.

FIG. 6 shows in vitro FIX expression in PCH. PCH were seeded in a 96-well plate and then treated with a constant 6.0E+4 vg/cell of the indicated insertion template (Insert 72, Insert 29, or Insert 30) packaged into rAAV8. LNP comprising Cas9 mRNA and one of four sgRNA targeting ALB intron 1 (g9860, g9857, g9874, and g9844) was added to replicate wells at concentrations ranging from 1000 ng/mL to 4.1 ng/mL across a 3-fold serial dilution series. Cells were incubated at 37° C., with media changes at day 3 and day 5, and hFIX expression was measured on day 8 via hFIX ELISA.

FIG. 7 shows measured expression of hFIX in plasma from ALB^{hu/hu}/F9^{-/-} mice treated with LNPF9 and/or REGV131. Plasma was isolated from mice at specified timepoints and quantified for hFIX levels using a detection antibody specific for hFIX (AHIX-5041; Haematologic Technologies) on the Meso Scale Device (MSD) platform.

FIG. 8A shows relative hFIX activity in plasma from ALB^{hu/hu}/F9^{-/-} mice treated with LNPF9 and/or REGV131. Activity was based on a chromogenic assay that specifically measures FIX activity.

FIG. 8B shows correlation of hFIX expression to relative hFIX activity in plasma from ALB^{hu/hu}/F9^{-/-} mice treated with LNPF9 and/or REGV131.

FIG. 9 shows IFN-I responses in a primary human plasmacytoid DC-based assay for non-CpG-depleted F9 insertion templates (Insert 72) and CpG-depleted F9 insertion templates (Insert 27, Insert 28, Insert 29, and Insert 30).

FIG. 10A shows measured expression of hFIX in plasma from Cynomolgus monkeys treated with LNPF9 (g9860) and/or REGV131 (Insert 30) or REGV013 (Insert 72). Plasma was isolated from monkeys at specified timepoints and quantified for hFIX levels using a detection antibody specific for hFIX (AHIX-5041; Haematologic Technologies) on the Meso Scale Device (MSD) platform.

FIG. 10B shows correlation of assessed hFIX levels to hFIX activity in plasma from monkeys treated with LNPF9 and/or REGV131 or REGV013. Human FIX (hFIX) proteins were isolated from the monkey plasma using a pull-down technique with an antibody specific for hFIX (AHIX-5041; Haematologic Technologies) and hFIX functional activity was determined with a chromogenic FIX activity assay (BioPhen FIX, Hypen BioMed).

FIG. 10C shows measured expression of hFIX in plasma from Cynomolgus monkeys treated with LNPF9 (g9860) and/or REGV131 (Insert 30) over a time course of 6 months. Plasma was isolated from monkeys at specified timepoints and hFIX levels were quantified using a detection antibody specific for hFIX (AHIX-5041; Haematologic Technologies) on the Meso Scale Device (MSD) platform.

FIG. 11 shows a schematic describing different human factor IX (hFIX) insertion templates tested in adult and neonatal mice.

FIG. 12A shows hFIX plasma levels in neonatal mice (n=4-10 per group; male and female) and adult mice (n=5 per group; female) at different time points post-administration of episomal hFIX (Episome), LNP-g666+hFIX-HDR-500 template (HDR500), LNP-g666+hFIX-HDR-800 template (HDR800), and LNP-g666+hFIX-NHEJ template (NHEJ). The administration in neonatal mice occurred at P0 or P1. Saline-injected mice were used as negative controls. Data are shown on a log scale. FIG. 12B shows hFIX plasma levels in neonatal mice (n=4-10 per group; male and female) at different time points post-administration of episomal hFIX (Episome), LNP-g666+hFIX-HDR-500 template (HDR500), LNP-g666+hFIX-HDR-800 template (HDR800), and LNP-g666+hFIX-NHEJ template (NHEJ). The administration in neonatal mice occurred at P0 or P1. Saline-injected mice were used as negative controls. Data are shown on a linear scale.

FIGS. 13A and 13B show that recombinant AAV8 hFIX viruses produced using 145 bp ITR, 141 bp ITR, and 130 bp ITR plasmids perform similarly in primary human hepatocytes. hFIX expression was measured by ELISA at day 7.

FIG. 14A shows measured expression of hFIX in plasma from ALB^{hu/hu}/F9^{-/-} mice treated with LNPF9 and/or REGV131. Plasma was isolated from mice at specified timepoints and hFIX levels were quantified using a detection antibody specific for hFIX (AHIX-5041; Haematologic Technologies) on the Meso Scale Device (MSD) platform. Values shown are mean+SEM.

FIG. 14B shows measured expression of hFIX in plasma from ALB^{hu/hu}/F9^{-/-} mice treated with LNPF9 and/or REGV131 at week 6 following treatment.

FIG. 14C shows measured expression of hFIX in plasma from ALB^{hu/hu}/F9^{-/-} mice treated with different combinations of doses of LNPF9 and/or REGV131 various time pointes following treatment.

FIG. 15A shows relative hFIX activity in plasma from ALB^{hu/hu}/F9^{-/-} mice treated with LNPF9 and/or REGV131. Plasma was isolated from mice at specified timepoints. Activity was based on a chromogenic assay that specifically measures FIX activity.

FIG. 15B shows relative hFIX activity in plasma from ALB^{hu/hu}/F9^{-/-} mice treated with LNPF9 and/or REGV131 at week 6 following treatment.

FIG. 15C shows correlation of hFIX expression to relative hFIX activity in plasma from ALB^{hu/hu}/F9^{-/-} mice treated with LNPF9 and REGV131. Data are plotted for individual animals as a correlation of level-to-activity.

FIG. 16 shows bleeding time after tail cut in ALB^{hu/hu}/F9^{-/-} mice treated with LNPF9 and/or REGV131 at 4 weeks after treatment. Values shown are mean+SEM.

FIG. 17 shows activated partial thromboplastin time (aPTT) time in undiluted plasma from ALB^{hu/hu}/F9^{-/-} mice treated with LNPF9 and/or REGV131 at 6 weeks after treatment. Values shown are mean+SEM.

FIG. 18 shows that mouse-generated human FIX from ALB^{hu/hu}/F9^{-/-} mice treated with LNPF9 and/or REGV131 demonstrates specific activity similar to purified human FIX in one-stage clotting/aPTT assays.

FIG. 19 shows that mouse-generated human FIX from ALB^{hu/hu}/F9^{-/-} mice treated with LNPF9 and/or REGV131 demonstrates specific activity similar to purified human FIX in two-stage chromogenic substrate (CS) assays.

### Definitions

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

Proteins are said to have an “N-terminus” and a “C-terminus.” The term “N-terminus” relates to the start of a protein or polypeptide, terminated by an amino acid with a free amine group (—NH2). The term “C-terminus” relates to the end of an amino acid chain (protein or polypeptide), terminated by a free carboxyl group (—COOH).

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

The term “genomically integrated” refers to a nucleic acid that has been introduced into a cell such that the nucleotide sequence integrates into the genome of the cell. Any protocol may be used for the stable incorporation of a nucleic acid into the genome of a cell.

The term “viral vector” refers to a recombinant nucleic acid that includes at least one element of viral origin and includes elements sufficient for or permissive of packaging into a viral vector particle. The vector and/or particle can be utilized for the purpose of transferring DNA, RNA, or other nucleic acids into cells in vitro, ex vivo, or in vivo. Numerous forms of viral vectors are known.

The term “isolated” with respect to cells, tissues (e.g., liver samples), proteins, and nucleic acids includes cells, tissues (e.g., liver samples), proteins, and nucleic acids that are relatively purified with respect to other bacterial, viral, cellular, or other components that may normally be present in situ, up to and including a substantially pure preparation of the cells, tissues (e.g., liver samples), proteins, and nucleic acids. The term “isolated” also includes cells, tissues (e.g., liver samples), proteins, and nucleic acids that have no naturally occurring counterpart, have been chemically synthesized and are thus substantially uncontaminated by other cells, tissues (e.g., liver samples), proteins, and nucleic acids, or has been separated or purified from most other components (e.g., cellular components) with which they are naturally accompanied (e.g., other cellular proteins, polynucleotides, or cellular components).

The term “wild type” includes entities having a structure and/or activity as found in a normal (as contrasted with mutant, diseased, altered, or so forth) state or context. Wild type genes and polypeptides often exist in multiple different forms (e.g., alleles).

The term “endogenous sequence” refers to a nucleic acid sequence that occurs naturally within a cell or animal. For example, an endogenous ALB sequence of a human refers to a native ALB sequence that naturally occurs at the ALB locus in the human.

“Exogenous” molecules or sequences include molecules or sequences that are not normally present in a cell in that form. Normal presence includes presence with respect to the particular developmental stage and environmental conditions of the cell. An exogenous molecule or sequence, for example, can include a mutated version of a corresponding endogenous sequence within the cell, such as a humanized version of the endogenous sequence, or can include a sequence corresponding to an endogenous sequence within the cell but in a different form (i.e., not within a chromosome). In contrast, endogenous molecules or sequences include molecules or sequences that are normally present in that form in a particular cell at a particular developmental stage under particular environmental conditions.

The term “heterologous” when used in the context of a nucleic acid or a protein indicates that the nucleic acid or protein comprises at least two segments that do not naturally occur together in the same molecule. For example, the term “heterologous,” when used with reference to segments of a nucleic acid or segments of a protein, indicates that the nucleic acid or protein comprises two or more sub-sequences that are not found in the same relationship to each other (e.g., joined together) in nature. As one example, a “heterologous” region of a nucleic acid vector is a segment of nucleic acid within or attached to another nucleic acid molecule that is not found in association with the other molecule in nature. For example, a heterologous region of a nucleic acid vector could include a coding sequence flanked by sequences not found in association with the coding sequence in nature. Likewise, a “heterologous” region of a protein is a segment of amino acids within or attached to another peptide molecule that is not found in association with the other peptide molecule in nature (e.g., a fusion protein, or a protein with a tag). Similarly, a nucleic acid or protein can comprise a heterologous label or a heterologous secretion or localization sequence.

“Codon optimization” (i.e., “codon optimized” sequences) takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding a factor IX protein can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Res. 28(1):292, herein incorporated by reference in its entirety for all purposes. Computer algorithms for codon optimization of a particular sequence for expression in a particular host are also available (see, e.g., Gene Forge).

The term “locus” refers to a specific location of a gene (or significant sequence), DNA sequence, polypeptide-encoding sequence, or position on a chromosome of the genome of an organism. For example, an “ALB locus” may refer to the specific location of an ALB gene, ALB DNA sequence, albumin-encoding sequence, or ALB position on a chromosome of the genome of an organism that has been identified as to where such a sequence resides. An “ALB locus” may comprise a regulatory element of an ALB gene, including, for example, an enhancer, a promoter, 5′ and/or 3′ untranslated region (UTR), or a combination thereof.

The term “gene” refers to DNA sequences in a chromosome that may contain, if naturally present, at least one coding and at least one non-coding region. The DNA sequence in a chromosome that codes for a product (e.g., but not limited to, an RNA product and/or a polypeptide product) can include the coding region interrupted with non-coding introns and sequence located adjacent to the coding region on both the 5′ and 3′ ends such that the gene corresponds to the full-length mRNA (including the 5′ and 3′ untranslated sequences). Additionally, other non-coding sequences including regulatory sequences (e.g., but not limited to, promoters, enhancers, and transcription factor binding sites), polyadenylation signals, internal ribosome entry sites, silencers, insulating sequence, and matrix attachment regions may be present in a gene. These sequences may be close to the coding region of the gene (e.g., but not limited to, within 10 kb) or at distant sites, and they influence the level or rate of transcription and translation of the gene.

The term “allele” refers to a variant form of a gene. Some genes have a variety of different forms, which are located at the same position, or genetic locus, on a chromosome. A diploid organism has two alleles at each genetic locus. Each pair of alleles represents the genotype of a specific genetic locus. Genotypes are described as homozygous if there are two identical alleles at a particular locus and as heterozygous if the two alleles differ.

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a mouse cell, a rat cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

“Operable linkage” or being “operably linked” includes juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

The methods and compositions provided herein employ a variety of different components. Some components throughout the description can have active variants and fragments. The term “functional” refers to the innate ability of a protein or nucleic acid (or a fragment or variant thereof) to exhibit a biological activity or function. The biological functions of functional fragments or variants may be the same or may in fact be changed (e.g., with respect to their specificity or selectivity or efficacy) in comparison to the original molecule, but with retention of the molecule's basic biological function.

The term “variant” refers to a nucleotide sequence differing from the sequence most prevalent in a population (e.g., by one nucleotide) or a protein sequence different from the sequence most prevalent in a population (e.g., by one amino acid).

The term “fragment,” when referring to a protein, means a protein that is shorter or has fewer amino acids than the full-length protein. The term “fragment,” when referring to a nucleic acid, means a nucleic acid that is shorter or has fewer nucleotides than the full-length nucleic acid. A fragment can be, for example, when referring to a protein fragment, an N-terminal fragment (i.e., removal of a portion of the C-terminal end of the protein), a C-terminal fragment (i.e., removal of a portion of the N-terminal end of the protein), or an internal fragment (i.e., removal of a portion of each of the N-terminal and C-terminal ends of the protein). A fragment can be, for example, when referring to a nucleic acid fragment, a 5′ fragment (i.e., removal of a portion of the 3′ end of the nucleic acid), a 3′ fragment (i.e., removal of a portion of the 5′ end of the nucleic acid), or an internal fragment (i.e., removal of a portion each of the 5′ and 3′ ends of the nucleic acid).

“Sequence identity” or “identity” in the context of two polynucleotides or polypeptide sequences refers to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values include the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized below.

TABLE 1
Amino Acid Categorizations.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

A “homologous” sequence (e.g., nucleic acid sequence) includes a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence. Homologous sequences can include, for example, orthologous sequence and paralogous sequences. Homologous genes, for example, typically descend from a common ancestral DNA sequence, either through a speciation event (orthologous genes) or a genetic duplication event (paralogous genes). “Orthologous” genes include genes in different species that evolved from a common ancestral gene by speciation. Orthologs typically retain the same function in the course of evolution. “Paralogous” genes include genes related by duplication within a genome. Paralogs can evolve new functions in the course of evolution.

The term “in vitro” includes artificial environments and to processes or reactions that occur within an artificial environment (e.g., a test tube or an isolated cell or cell line). The term “in vivo” includes natural environments (e.g., a cell or organism or body) and to processes or reactions that occur within a natural environment. The term “ex vivo” includes cells that have been removed from the body of an individual and processes or reactions that occur within such cells.

As used herein, the term “neonatal” in the context of humans covers human subjects up to or under the age of 1 year (52 weeks), preferably up to or under the age of 24 weeks, more preferably up to or under the age of 12 weeks, more preferably up to or under the age of 8 weeks, and even more preferably up to or under the age of 4 weeks. In certain embodiments, a neonatal human subject is up to 4 weeks of age. In certain embodiments, a neonatal human subject is up to 8 weeks of age. In another embodiment, a neonatal human subject is within 3 weeks after birth. In another embodiment, a neonatal human subject is within 2 weeks after birth. In another embodiment, a neonatal human subject is within 1 week after birth. In another embodiment, a neonatal human subject is within 7 days after birth. In another embodiment, a neonatal human subject is within 6 days after birth. In another embodiment, a neonatal human subject is within 5 days after birth. In another embodiment, a neonatal human subject is within 4 days after birth. In another embodiment, a neonatal human subject is within 3 days after birth. In another embodiment, a neonatal human subject is within 2 days after birth. In another embodiment, a neonatal human subject is within 1 day after birth. The time windows disclosed above are for human subjects and are also meant to cover the corresponding developmental time windows for other animals. As used herein, a “neonatal cell” is a cell of a neonatal subject, and a population of neonatal cells is a population of cells of a neonatal subject.

As used herein, a “control” as in a control sample or a control subject is a comparator for a measurement, e.g., a diagnostic measurement of a sign or symptom of a disease. In certain embodiments, a control can be a subject sample from the same subject an earlier time point, e.g., before a treatment intervention. In certain embodiments, a control can be a measurement from a normal subject, i.e., a subject not having the disease of the treated subject, to provide a normal control, e.g., FIX concentration or activity in a subject sample. In certain embodiments, a normal control can be a population control, i.e., the average of subjects in the general population. In certain embodiments, a control can be an untreated subject with the same disease. In certain embodiments, a control can be a subject treated with a different therapy, e.g., the standard of care. In certain embodiments, a control can be a subject or a population of subjects from a natural history study of subjects with the disease of the subject being compared. In certain embodiments, the control is matched for certain factors to the subject being tested, e.g., age, gender. In certain embodiments, a control may be a control level for a particular lab, e.g., a clinical lab. Selection of an appropriate control is within the ability of those of skill in the art.

Compositions or methods “comprising” or “including” one or more recited elements may include other elements not specifically recited. For example, a composition that “comprises” or “includes” a protein may contain the protein alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which the event or circumstance does not.

Designation of a range of values includes all integers within or defining the range, and all subranges defined by integers within the range. For example, 5-10 nucleotides is understood as 5, 6, 7, 8, 9, or 10 nucleotides, whereas 5-10% is understood to contain 5% and all possible values through 10%.

At least 17 nucleotides of a 20 nucleotide sequence is understood to include 17, 18, 19, or 20 nucleotides of the sequence provided, thereby providing a upper limit even if one is not specifically provided as it would be clearly understood. Similarly, up to 3 nucleotides would be understood to encompass 0, 1, 2, or 3 nucleotides, providing a lower limit even if one is not specifically provided. When “at least”, “up to”, or other similar language modifies a number, it can be understood to modify each number in the series.

As used herein, “no more than” or “less than” is understood as the value adjacent to the phrase and logical lower values or integers, as logical from context, to zero. For example, a duplex region of “no more than 2 nucleotide base pairs” has a 2, 1, or 0 nucleotide base pairs. When “no more than” or “less than” is present before a series of numbers or a range, it is understood that each of the numbers in the series or range is modified.

As used herein, it is understood that when the maximum amount of a value is represented by 100% (e.g., 100% inhibition or 100% encapsulation) that the value is limited by the method of detection. For example, 100% inhibition is understood as inhibition to a level below the level of detection of the assay, and 100% encapsulation is understood as no material intended for encapsulation can be detected outside the vesicles.

Unless otherwise apparent from the context, the term “about” encompasses values ±5% of a stated value. In certain embodiments, the term “about” is understood to encompass tolerated variation or error within the art, e.g., 2 standard deviations from the mean, or the sensitivity of the method used to take a measurement, or a percent of a value as tolerated in the art, e.g., with age. When “about” is present before the first value of a series, it can be understood to modify each value in the series.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p≤0.05.

In the event of a conflict between a sequence in the application and an indicated accession number or position in an accession number, the sequence in the application predominates.

### DETAILED DESCRIPTION

### I. Overview

Provided herein are nucleic acid constructs and compositions that allow insertion of a factor IX (FIX) coding sequence into a target genomic locus such as an endogenous albumin (ALB) locus and/or expression of the FIX coding sequence. The nucleic acid constructs and compositions can be used in methods of introducing a factor 9 (F9) nucleic acid into a cell, methods of integration of a F9 nucleic acid into a target genomic locus, methods of expression of FIX in a cell, and in methods of treating hemophilia B or FIX deficiency in a subject. In some cases, the cells or subjects can be neonatal cells or neonatal subjects as defined herein. In other cases, the cells are not neonatal cells, and the subjects are not neonatal subjects. Also provided are nuclease agents (e.g., targeting an endogenous ALB locus) or nucleic acids encoding nuclease agents to facilitate integration of the nucleic acid constructs into a target genomic locus such as an endogenous ALB locus.

More specifically, described herein in some embodiments is a therapeutic product based on the CRISPR/Cas9 gene editing technology and optionally contained in a lipid nanoparticle (LNP) delivery system, associated with a F9 DNA gene insertion template (e.g., a unidirectional or bidirectional F9 DNA gene insertion template) optionally contained in a recombinant adeno-associated virus serotype 8 (rAAV8). The CRISPR/Cas9 component has been designed to target and cut the double stranded DNA at a target gene locus (e.g., a safe harbor locus such as an ALB gene locus in hepatocytes), allowing for the F9 DNA template to be inserted in the genome at the target genomic locus. Transgene insertion provides a functional F9 gene, encoding the missing or defective genomic F9 in hemophilia B patients.

In some cases, the FIX coding sequences in the constructs disclosed herein are optimized for expression as compared to native FIX coding sequence. In other cases, the FIX coding sequences in the constructs disclosed herein can comprise native FIX coding sequences. For example, the FIX coding sequences in the constructs disclosed herein may include one or more modifications such as codon optimization (e.g., to human codons), depletion of CpG dinucleotides, mutation of cryptic splice sites, or any combination thereof. In one specific example, a FIX coding sequence in a construct disclosed herein has one or more CpG dinucleotides removed (i.e., is CpG depleted) and has one or more cryptic splice sites mutated or removed as compared to a native FIX coding sequence. In another specific example, a FIX coding sequence in a construct disclosed herein has all but one CpG dinucleotides removed (e.g., introducing one CpG to mutate a cryptic splice site) and has one or more or all identified cryptic splice sites mutated or removed as compared to a native FIX coding sequence. In another specific example, a FIX coding sequence in a construct disclosed herein has one or more CpG dinucleotides removed (i.e., is CpG depleted) and is codon optimized (e.g., codon optimized for expression in a human or mammal) as compared to a native FIX coding sequence. In another specific example, a FIX coding sequence in a construct disclosed herein has all CpG dinucleotides removed (i.e., is fully CpG depleted) and is codon optimized (e.g., codon optimized for expression in a human or mammal) as compared to a native FIX coding sequence.

In particular, provided herein are bidirectional constructs comprising two different FIX coding sequences (i.e., a first FIX coding sequence and a reverse complement of a second FIX coding sequence). In one example of such a bidirectional construct, one FIX coding sequence has one or more CpG dinucleotides removed (i.e., is CpG depleted) and is codon optimized (e.g., codon optimized for expression in a human or mammal) as compared to a native FIX coding sequence (e.g., has all CpG dinucleotides removed (i.e., is fully CpG depleted) and is codon optimized (e.g., codon optimized for expression in a human or mammal) as compared to a native FIX coding sequence), and the other FIX coding sequence has one or more CpG dinucleotides removed (i.e., is CpG depleted) and has one or more cryptic splice sites mutated or removed as compared to a native FIX coding sequence (e.g., has all but one CpG dinucleotides removed (e.g., introducing one CpG to mutate a cryptic splice site) and has one or more or all identified cryptic splice sites mutated or removed as compared to a native FIX coding sequence).

One particular example of a FIX coding sequence that is codon optimized and fully CpG depleted is set forth in SEQ ID NO: 166. Another particular example of a FIX coding sequence that is codon optimized and fully CpG depleted is set forth in SEQ ID NO: 165. One particular example of an optimized native FIX coding sequence that has all but one CpG dinucleotides removed and is modified to mutate one or more cryptic splice sites is set forth in SEQ ID NO: 159. One particular example of a bidirectional construct comprising two different FIX coding sequences (i.e., a first FIX coding sequence and a reverse complement of a second FIX coding sequence) includes the FIX coding sequences set forth in SEQ ID NOS: 166 and 159. Another particular example of a bidirectional construct comprising two different FIX coding sequences (i.e., a first FIX coding sequence and a reverse complement of a second FIX coding sequence) includes the FIX coding sequences set forth in SEQ ID NOS: 165 and 159.

Also provided herein are unidirectional constructs comprising a single FIX coding sequence. In one example of such a unidirectional construct, the FIX coding sequence has one or more CpG dinucleotides removed (i.e., is CpG depleted) and is codon optimized (e.g., codon optimized for expression in a human or mammal) as compared to a native FIX coding sequence (e.g., has all CpG dinucleotides removed (i.e., is fully CpG depleted) and is codon optimized (e.g., codon optimized for expression in a human or mammal) as compared to a native FIX coding sequence). In another example of such a unidirectional construct, the FIX coding sequence has one or more CpG dinucleotides removed (i.e., is CpG depleted) and has one or more cryptic splice sites mutated or removed as compared to a native FIX coding sequence (e.g., has all but one CpG dinucleotides removed (e.g., introducing one CpG to mutate a cryptic splice site) and has one or more or all identified cryptic splice sites mutated or removed as compared to a native FIX coding sequence).

One particular example of a FIX coding sequence that is codon optimized and fully CpG depleted is set forth in SEQ ID NO: 166. Another particular example of a FIX coding sequence that is codon optimized and fully CpG depleted is set forth in SEQ ID NO: 165. One particular example of an optimized native FIX coding sequence that has all but one CpG dinucleotides removed and is modified to mutate one or more cryptic splice sites is set forth in SEQ ID NO: 159. One particular example of a unidirectional construct comprises the FIX coding sequence set forth in SEQ ID NOS: 166. Another particular example of a unidirectional construct comprises the FIX coding sequence set forth in SEQ ID NOS: 165. Another particular example of a unidirectional construct comprises the FIX coding sequence set forth in SEQ ID NOS: 159.

Compared to previous generation FIX coding sequences and previous generation bidirectional F9 insertion cassettes, the constructs disclosed herein maintain FIX expression (levels, activity, low variability, manufacturability) while also removing potential negative features that might impact template performance in human patients, minimizing unintended cryptic splicing events observed in wild type mice and non-human primates, and depleting CpGs because CpG sites in AAV viral vectors contain unmethylated C residues, which can be potent TLR9 agonists.

The gene insertion platform described herein also has advantages over existing episomal FIX platforms. For example, there are no concerns regarding integration of promoter-containing constructs because the F9 insertion templates used were promoterless. Because there are no promoter/regulatory elements in the AAV cassettes used, they are less likely to influence expression of neighboring loci if randomly inserted. Likewise, because there is no in-frame ATG/methionine at the 5′ end of the cassettes used, no protein is produced from the template alone. In addition, there is an extremely low likelihood of producing protein from off-target insertion because it would require intronic insertion leading to splicing into the correct reading frame. Another advantage over existing F9 episome platforms is that robust protein expression was achieved in vivo by harnessing transcription from a highly active genomic locus, so there is no requirement for using a hyperactive variant of FIX to achieve therapeutic activity. In contrast, episomal FIX platforms currently in clinical trials encode hyperactive mutant variants of FIX in order to achieve therapeutic activity levels. The compositions in methods disclosed herein, however, lead to high levels of expression, even when wild type mature FIX is encoded without any artificial hyperactive mutations. Moreover, integration of the coding sequence as in the compositions and methods disclosed herein is advantageous over non-integrating episomal vectors because transgene retention over time can be problematic with non-replicating episomal vectors, making it necessary to administer more virus for continued therapeutic response. However, these subsequent exposures may result in rapid neutralization of the virus and, therefore, decreased transgene expression. This is not an issue with the compositions and methods disclosed herein—no redosing is required because the compositions and methods result in integration into the genome and permanent expression.

### II. Compositions for Expressing Factor IX

Provided herein are nucleic acid constructs and compositions that allow insertion of a Factor IX (FIX) coding sequence into a target genomic locus such as an endogenous albumin (ALB) locus and/or expression of the FIX coding sequence. The nucleic acid constructs and compositions can be used in methods for integration into a target genomic locus and/or expression in a cell or in methods of treating hemophilia B or FIX deficiency. Also provided are nuclease agents (e.g., targeting an endogenous ALB locus) or nucleic acids encoding nuclease agents to facilitate integration of the nucleic acid constructs into a target genomic locus such as an endogenous ALB locus.

A. Factor 9 Nucleic Acid Constructs

The compositions and methods described herein include the use of a nucleic acid construct that comprises a FIX protein coding sequence (a factor 9 (F9) nucleic acid) or a reverse complement of the FIX protein coding sequence (e.g., a heterologous FIX protein coding sequence (a heterologous F9 nucleic acid) or a reverse complement of the heterologous FIX protein coding sequence). For example, the nucleic acid construct can comprise a FIX protein coding sequence (a F9 nucleic acid), such as a heterologous FIX protein coding sequence (e.g., a heterologous F9 nucleic acid). Such nucleic acid constructs can be for insertion into a target genomic locus following cleavage by a nuclease agent or CRISPR/Cas system as disclosed elsewhere herein or can be for expression of FIX without insertion into a target genomic locus (e.g., in an episome). For example, such nucleic acid constructs can be for insertion into a cleavage site created by a nuclease agent or CRISPR/Cas system as disclosed elsewhere herein or can be for expression of FIX without insertion into a cleavage site (e.g., in an episome). The term cleavage site includes a DNA sequence at which a nick or double-strand break is created by a nuclease agent (e.g., a Cas9 protein complexed with a guide RNA). A “heterologous” FIX protein coding sequence can refer to a coding sequence that has been introduced as an exogenous source to a site within a host cell genome (e.g., at a genomic locus such as a safe harbor locus, including ALB intron 1). That is, the heterologous protein coding sequence is heterologous with respect to its insertion site, and the polypeptide expressed from such a heterologous coding sequence is referred to as a heterologous polypeptide. The heterologous coding sequence can be naturally-occurring or engineered, and can be wild type or a variant. The heterologous coding sequence may include nucleotide sequences other than the sequence that encodes the heterologous polypeptide (e.g., an internal ribosomal entry site). The heterologous coding sequence can be a coding sequence that occurs naturally in the host genome, as a wild type or a variant (e.g., mutant). For example, although the host cell contains the coding sequence of interest (as a wild type or as a variant), the same coding sequence or variant thereof can be introduced as an exogenous source (e.g., for expression at a locus that is highly expressed). The heterologous coding sequence can also be a coding sequence that is not naturally occurring in the host genome, or that expresses a heterologous polypeptide that does not naturally occur in the host genome. A heterologous coding sequence can include an exogenous nucleic acid sequence (e.g., a nucleic acid sequence is not endogenous to the recipient cell), or may be heterologous with respect to its insertion site and/or with respect to its recipient cell.

The length of the F9 nucleic acid constructs disclosed herein can vary. The construct can be, for example, from about 1 kb to about 5 kb, such as from about 1 kb to about 4.5 kb or about 1 kb to about 4 kb. An exemplary nucleic acid construct is between about 1 kb to about 5 kb in length or between about 1 kb to about 4 kb in length. Alternatively, a nucleic acid construct can be between about 1 kb to about 1.5 kb, about 1.5 kb to about 2 kb, about 2 kb to about 2.5 kb, about 2.5 kb to about 3 kb, about 3 kb to about 3.5 kb, about 3.5 kb to about 4 kb, about 4 kb to about 4.5 kb, or about 4.5 kb to about 5 kb in length. Alternatively, a nucleic acid construct can be, for example, no more than 5 kb, no more than 4.5 kb, no more than 4 kb, no more than 3.5 kb, no more than 3 kb, or no more than 2.5 kb in length.

The constructs can comprise deoxyribonucleic acid (DNA) or ribonucleic acid (RNA), can be single-stranded, double-stranded, or partially single-stranded and partially double-stranded, and can be introduced into a host cell in linear or circular (e.g., minicircle) form. See, e.g., US 2010/0047805, US 2011/0281361, and US 2011/0207221, each of which is herein incorporated by reference in their entirety for all purposes. If introduced in linear form, the ends of the construct can be protected (e.g., from exonucleolytic degradation) by known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in their entirety for all purposes. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. A construct can be introduced into a cell as part of a vector molecule having additional sequences such as, for example, replication origins, promoters, and genes encoding antibiotic resistance. A construct may omit viral elements. Moreover, constructs can be introduced as a naked nucleic acid, can be introduced as a nucleic acid complexed with an agent such as a liposome or poloxamer, or can be delivered by viruses (e.g., adenovirus, adeno-associated virus (AAV), herpesvirus, retrovirus, or lentivirus).

The constructs disclosed herein can be modified on either or both ends to include one or more suitable structural features as needed and/or to confer one or more functional benefit. For example, structural modifications can vary depending on the method(s) used to deliver the constructs disclosed herein to a host cell (e.g., use of viral vector delivery or packaging into lipid nanoparticles for delivery). Such modifications include, for example, terminal structures such as inverted terminal repeats (ITR), hairpin, loops, and other structures such as toroids. For example, the constructs disclosed herein can comprise one, two, or three ITRs or can comprise no more than two ITRs. Various methods of structural modifications are known.

Some constructs may be inserted so that their expression is driven by the endogenous promoter at the insertion site (e.g., the endogenous ALB promoter when the construct is integrated into the host cell's ALB locus). Such constructs may not comprise a promoter that drives the expression of FIX. For example, the expression of FIX can be driven by a promoter of the host cell (e.g., the endogenous ALB promoter when the transgene is integrated into a host cell's ALB locus). In such cases, the construct may lack control elements (e.g., promoter and/or enhancer) that drive its expression (e.g., a promoterless construct). Nonetheless, in other cases the construct may comprise a promoter and/or enhancer, for example a constitutive promoter or an inducible or tissue-specific (e.g., liver- or platelet-specific) promoter that drives expression of the FIX in an episome or upon integration. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EF1a) promoter, ubiquitin promoters, actin promoters, tubulin promoters, immunoglobulin promoters, a functional fragment thereof, or a combination of any of the foregoing. For example, the promoter may be a CMV promoter or a truncated CMV promoter. In another example, the promoter may be an EF1a promoter. Non-limiting exemplary inducible promoters include those inducible by heat shock, light, chemicals, peptides, metals, steroids, antibiotics, or alcohol. The inducible promoter may be one that has a low basal (non-induced) expression level, such as the Tet-On® promoter (Clontech). Although not required for expression, the constructs may comprise transcriptional or translational regulatory sequences such as promoters, enhancers, insulators, internal ribosome entry sites, additional sequences encoding peptides, and/or polyadenylation signals. The construct may comprise a sequence encoding a heterologous FIX protein downstream of and operably linked to a signal sequence encoding a signal peptide. In some examples, the nucleic acid construct works in homology-independent insertion of a nucleic acid that encodes a FIX protein. Such nucleic acid constructs can work, for example, in non-dividing cells (e.g., cells in which non-homologous end joining (NHEJ), not homologous recombination (HR), is the primary mechanism by which double-stranded DNA breaks are repaired). Such constructs can be, for example, homology-independent donor constructs. Such nucleic acid constructs can work, for example, in dividing cells (e.g., actively dividing cells).

The constructs disclosed herein can be modified to include or exclude any suitable structural feature as needed for any particular use and/or that confers one or more desired function. For example, some constructs disclosed herein do not comprise a homology arm. Some constructs disclosed herein are capable of insertion into a cut site in a target DNA sequence for a nuclease agent (e.g., capable of insertion into a safe harbor gene, such as an ALB locus) by non-homologous end joining. Some such constructs do not comprise homology arms. For example, such constructs can be inserted into a blunt end double-strand break following cleavage with a nuclease agent (e.g., CRISPR/Cas system) as disclosed herein. In a specific example, the construct can be delivered via AAV and can be capable of insertion by non-homologous end joining (e.g., the construct can be one that does not comprise homology arms).

In a particular example, the construct can be inserted via homology-independent targeted integration. For example, the heterologous F9 nucleic acid in the construct can be flanked on each side by a target site for a nuclease agent (e.g., the same target site as in the target DNA sequence for targeted insertion (e.g., in a safe harbor gene), and the same nuclease agent being used to cleave the target DNA sequence for targeted insertion). The nuclease agent can then cleave the target sites flanking the heterologous F9 nucleic acid. In a specific example, the construct is delivered AAV-mediated delivery, and cleavage of the target sites flanking the heterologous F9 nucleic acid can remove the inverted terminal repeats (ITRs) of the AAV. In some instances, the target DNA sequence for targeted insertion (e.g., target DNA sequence in a safe harbor locus such as a gRNA target sequence including the flanking protospacer adjacent motif) is no longer present if the heterologous F9 nucleic acid is inserted into the cut site or target DNA sequence in the correct orientation but it is reformed if the heterologous F9 nucleic acid is inserted into the cut site or target DNA sequence in the opposite orientation. This can help ensure that the heterologous F9 nucleic acid is inserted in the correct orientation for expression.

The constructs disclosed herein can comprise a polyadenylation tail sequence (e.g., downstream or 3′ of a FIX coding sequence). Methods of designing a suitable polyadenylation tail sequence are well-known. The polyadenylation tail sequence can be encoded, for example, as a “poly-A” stretch downstream of the FIX coding sequence. A poly-A tail can comprise, for example, at least 20, 30, 40, 50, 60, 70, 80, 90, or 100 adenines, and optionally up to 300 adenines. In a specific example, the poly-A tail comprises 95, 96, 97, 98, 99, or 100 adenine nucleotides. Methods of designing a suitable polyadenylation tail sequence and/or polyadenylation signal sequence are well known. For example, the polyadenylation signal sequence AAUAAA is commonly used in mammalian systems, although variants such as UAUAAA or AU/GUAAA have been identified. See, e.g., Proudfoot (2011) Genes & Dev. 25(17):1770-82, herein incorporated by reference in its entirety for all purposes. The term polyadenylation signal sequence refers to any sequence that directs termination of transcription and addition of a poly-A tail to the mRNA transcript. In eukaryotes, transcription terminators are recognized by protein factors, and termination is followed by polyadenylation, a process of adding a poly(A) tail to the mRNA transcripts in presence of the poly(A) polymerase. The mammalian poly(A) signal typically consists of a core sequence, about 45 nucleotides long, that may be flanked by diverse auxiliary sequences that serve to enhance cleavage and polyadenylation efficiency. The core sequence consists of a highly conserved upstream element (AATAAA or AAUAAA) in the mRNA, referred to as a poly A recognition motif or poly A recognition sequence), recognized by cleavage and polyadenylation-specificity factor (CPSF), and a poorly defined downstream region (rich in Us or Gs and Us), bound by cleavage stimulation factor (CstF). Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOX1 transcription termination sequence, a CYC1 transcription termination sequence, or any transcription termination sequence known to be suitable for regulating gene expression in eukaryotic cells. In one example, the polyadenylation signal is a simian virus 40 (SV40) late polyadenylation signal. For example, the polyadenylation signal can comprise, consist essentially of, or consist of SEQ ID NO: 199. In another example, the polyadenylation signal is a bovine growth hormone (BGH) polyadenylation signal or a CpG depleted BGH polyadenylation signal. For example, the polyadenylation signal can comprise, consist essentially of, or consist of SEQ ID NO: 200.

The constructs disclosed herein may also comprise splice acceptor sites (e.g., operably linked to the FIX coding sequence, such as upstream or 5′ of the FIX coding sequence). The splice acceptor site can, for example, comprise NAG or consist of NAG. In a specific example, the splice acceptor is an ALB splice acceptor (e.g., an ALB splice acceptor used in the splicing together of exons 1 and 2 of ALB (i.e., ALB exon 2 splice acceptor)). For example, such a splice acceptor can be derived from the human ALB gene. In another example, the splice acceptor can be derived from the mouse Alb gene (e.g., an ALB splice acceptor used in the splicing together of exons 1 and 2 of mouse Alb (i.e., mouse Alb exon 2 splice acceptor)). In another example, the splice acceptor is a F9 splice acceptor (e.g., the F9 splice acceptor used in the splicing together of exons 1 and 2 of F9). For example, such a splice acceptor can be derived from the human F9 gene. Alternatively, such a splice acceptor can be derived from the mouse F9 gene. Additional suitable splice acceptor sites useful in eukaryotes, including artificial splice acceptors, are well-known. See, e.g., Shapiro et al. (1987) Nucleic Acids Res. 15:7155-7174 and Burset et al. (2001) Nucleic Acids Res. 29:255-259, each of which is herein incorporated by reference in its entirety for all purposes. In a specific example, the splice acceptor is a mouse Alb exon 2 splice acceptor. In a specific example, the splice acceptor can comprise, consist essentially of, or consist of SEQ ID NO: 201.

In some examples, the nucleic acid constructs disclosed herein can be bidirectional constructs, which are described in more detail below. In some examples, the nucleic acid constructs disclosed herein can be unidirectional constructs, which are described in more detail below. Likewise, in some examples, the nucleic acid constructs disclosed herein can be in a vector (e.g., viral vector, such as AAV, or rAAV8) and/or a lipid nanoparticle as described in more detail elsewhere herein.

(1) Factor 9 (F9)

Coagulation factor IX (FIX; also known as Christmas factor or plasma thromboplastin component or PTC) is encoded by factor 9 (F9) and is a 415-amino acid serine protease synthesized in the liver. It is a vitamin K-dependent plasma protein that participates in the intrinsic pathway of blood coagulation by converting factor X to its active form in the presence of Ca²⁺ ions, phospholipids, and factor VIIIa. The plasma concentration of FIX is about 50 times that of factor VIII, and FIX has a half-life of about 24 hours.

The FIX expressed from the compositions and methods disclosed herein can be any wild type or variant FIX. In one example, the FIX is a human FIX protein. Human FIX is assigned UniProt reference number P00740. An exemplary amino acid sequence for human Factor IX is assigned NCBI Accession No. NP_000124.1 and is set forth in SEQ ID NO: 1. An exemplary human F9 mRNA (cDNA) sequence is assigned NCBI Accession No. NM_000133.4 and is set forth in SEQ ID NO: 2. An exemplary human F9 coding sequence is assigned CCDS ID CCDS14666.1 and is set forth in SEQ ID NO: 3.

In some examples, the FIX (e.g., human FIX) is a wild type FIX (e.g., wild type human FIX) sequence or a fragment thereof. For example, the FIX can be a fragment comprising the mature FIX amino acid sequence (i.e., the FIX sequence after removal of the signal peptide and propeptide), or a fragment comprising the mature FIX amino acid sequence and a portion of the propeptide. In a specific example, the FIX can comprise SEQ ID NO: 195 or can be at least 90%, at least 91%, at least 92%, at least 93%, at least 94%, at least 95%, at least 96%, at least 97%, at least 98%, at least 99%, or at least 99.5% identical to SEQ ID NO: 195.

In some examples, the FIX (e.g., human FIX) is not a hyperactive or hyperfunctional variant of FIX (i.e., the FIX does not have one or more mutations that increase the activity of the variant FIX relative to wild type). In other examples, the FIX (e.g., human FIX) is not a FVIII-independent variant of FIX (i.e., the FIX does not have one or more mutations that allow the variant FIX to activate coagulation in the absence of its cofactor, factor VIII). In other examples, the FIX (e.g., human FIX) is not a hyperactive or hyperfunctional variant of FIX and is not a FVIII-independent variant of FIX.

In other examples, the FIX (e.g., human FIX) is a variant FIX (e.g., a variant human FIX) or a fragment thereof. For example, the variant FIX or fragment thereof can comprise one or more mutations. In one example, the variant FIX or fragment thereof can have one or more mutations that increase the activity of the variant FIX (hyperactive or hyperfunctional) relative to wild type, such as an amino acid substitution in position R338 (e.g., R338A or R338L) and/or an amino acid substitution at position 5377 (e.g., S377W). See, e.g., US 2019/0017039 and US 2020/0172892, each of which is herein incorporated by reference in its entirety for all purposes. The numbering referred to herein is the standard FIX numbering, with position 1 being the tyrosine at amino acid 47 in SEQ ID NO: 1 (i.e., the first amino acid of the mature FIX protein following the signal peptide and propeptide in SEQ ID NO: 1). Further examples of variant FIX comprise an amino acid at residue 338 chosen from alanine, leucine, valine, isoleucine, phenylalanine, tryptophan, methionine, serine, and threonine. Further FIX variants comprise an amino acid at residue 338 chosen from leucine, cysteine, aspartic acid, glutamic acid, histidine, lysine, asparagine, glutamine, or tyrosine. In another example, the variant FIX or fragment thereof can have one or more mutations that allow the variant FIX to activate coagulation in the absence of its cofactor, factor VIII, such as an amino acid substitution at position L6, V181, E185, Y259, A261, K265, Y345, I383, E388, or a combination thereof (e.g., L6F, V181I, E185D, E185S, Y259F, A261K, K265A, K265T, Y345F, I383V, E188G, or a combination thereof). See, e.g., U.S. Pat. Nos. 10,125,357, 10,000,748, 10,604,749, US 2008/0214462, U.S. Pat. Nos. 8,022,187, and 8,513,386, each of which is herein incorporated by reference in its entirety for all purposes. In another example, the variant FIX or fragment thereof can have one or more mutations that allow the variant FIX to activate coagulation in the absence of its cofactor, factor VIII, such as an amino acid substitution at position V181, K265, I383, or a combination thereof or at position L6, V181, K265, I383, E185, or a combination thereof (e.g., an L6F mutation, a V181I mutation, a K265A or K265T mutation, an I383V mutation, an E185D mutation, or a combination thereof such as L6F/V181I/K265A/I383V, L6F/V181I/K265T/I383V, V181I/K265A/I383V/E185D, V181I/K265T/I383V/E185D, V181I/K265A/I383V/E185S, or V181I/K265T/I383V/E185S, or a V181I mutation, a K265A or K265T mutation, an I383V mutation, or a combination thereof such as V181I/K265A/I383V or V181I/K265T/I383V). In another example, the variant FIX or fragment thereof can have one or more mutations that increase the activity of the variant FIX relative to wild type and one or more mutations that allow the variant FIX to activate coagulation in the absence of its cofactor, factor VIII.

The FIX coding sequences in the constructs disclosed herein may include wild type FIX coding sequences without any modifications. The FIX coding sequences in the constructs disclosed herein may include one or more modifications such as codon optimization (e.g., to human codons), depletion of CpG dinucleotides, mutation of cryptic splice sites, addition of one or more glycosylation sites, or any combination thereof. CpG dinucleotides in a construct can limit the therapeutic utility of the construct. First, unmethylated CpG dinucleotides can interact with host toll-like receptor-9 (TLR-9) to stimulate innate, proinflammatory immune responses. Second, once the CpG dinucleotides become methylated, they can result in the suppression of transgene expression coordinated by methyl-CpG binding proteins. Cryptic splice sites are sequences in a pre-messenger RNA that are not normally used as splice sites, but that can be activated, for example, by mutations that either inactivate canonical splice sites or create splice sites where one did not exist before. Accurate splice site selection is critical for successful gene expression, and removal of cryptic splice sites can favor use of the normal or intended splice site.

In one example, a FIX coding sequence in a construct disclosed herein has one or more cryptic splice sites mutated or removed. In another example, a FIX coding sequence in a construct disclosed herein has all identified cryptic splice sites mutated or removed. In another example, a FIX coding sequence in a construct disclosed herein has one or more CpG dinucleotides removed (i.e., is CpG depleted). In another example, a FIX coding sequence in a construct disclosed herein has all but one CpG dinucleotides removed. In another example, a FIX coding sequence in a construct disclosed herein has all CpG dinucleotides removed (i.e., is fully CpG depleted). In another example, a FIX coding sequence in a construct disclosed herein is codon optimized (e.g., codon optimized for expression in a human or mammal). In a specific example, a FIX coding sequence in a construct disclosed herein has one or more CpG dinucleotides removed (i.e., is CpG depleted) and has one or more cryptic splice sites mutated or removed. In another specific example, a FIX coding sequence in a construct disclosed herein has all but one CpG dinucleotides removed (e.g., introducing one CpG to mutate a cryptic splice site) and has one or more or all identified cryptic splice sites mutated or removed. In another specific example, a FIX coding sequence in a construct disclosed herein has one or more CpG dinucleotides removed (i.e., is CpG depleted) and is codon optimized (e.g., codon optimized for expression in a human or mammal). In another specific example, a FIX coding sequence in a construct disclosed herein has all CpG dinucleotides removed (i.e., is fully CpG depleted) and is codon optimized (e.g., codon optimized for expression in a human or mammal).

Various codon optimized FIX coding sequences are provided. The FIX coding sequence can be, for example, CpG-depleted (e.g., fully CpG depleted) and/or codon optimized (e.g., CpG depleted (e.g., fully CpG-depleted) and codon optimized). In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 162-171. In another example, the FIX coding sequence is (or comprises a sequence) at least 95%, at least 96%, at least 97%, at least 98%, at least 99%, at least 99.5%, or 100% identical to any one of SEQ ID NOS: 162-171. In another example, the FIX coding sequence is (or comprises a sequence) at least 99%, at least 99.5%, or 100% identical to any one of SEQ ID NOS: 162-171. In another example, the FIX coding sequence comprises the sequence set forth in any one of SEQ ID NOS: 162-171. In another example, the FIX coding sequence consists essentially of the sequence set forth in any one of SEQ ID NOS: 162-171. In another example, the FIX coding sequence consists of the sequence set forth in any one of SEQ ID NOS: 162-171. Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, the FIX coding sequence in the above examples encodes a FIX protein comprising the sequence set forth in SEQ ID NO: 195. Optionally, the FIX coding sequence in the above examples encodes a FIX protein consisting essentially of the sequence set forth in SEQ ID NO: 195. Optionally, the FIX coding sequence in the above examples encodes a FIX protein consisting of the sequence set forth in SEQ ID NO: 195.

In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 164-171. In another example, the FIX coding sequence is (or comprises a sequence) at least 95%, at least 96%, at least 97%, at least 98%, at least 99%, at least 99.5%, or 100% identical to any one of SEQ ID NOS: 164-171. In another example, the FIX coding sequence is (or comprises a sequence) at least 99%, at least 99.5%, or 100% identical to any one of SEQ ID NOS: 164-171. In another example, the FIX coding sequence comprises the sequence set forth in any one of SEQ ID NOS: 164-171. In another example, the FIX coding sequence consists essentially of the sequence set forth in any one of SEQ ID NOS: 164-171. In another example, the FIX coding sequence consists of the sequence set forth in any one of SEQ ID NOS: 164-171. The FIX coding sequence can be, for example, CpG-depleted (e.g., fully CpG-depleted) and/or codon optimized. For example, the FIX coding sequence can be CpG depleted (e.g., fully CpG-depleted) and codon optimized. Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, the FIX coding sequence in the above examples encodes a FIX protein comprising the sequence set forth in SEQ ID NO: 195. Optionally, the FIX coding sequence in the above examples encodes a FIX protein consisting essentially of the sequence set forth in SEQ ID NO: 195. Optionally, the FIX coding sequence in the above examples encodes a FIX protein consisting of the sequence set forth in SEQ ID NO: 195.

In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 166 or 165. In another example, the FIX coding sequence is (or comprises a sequence) at least 95%, at least 96%, at least 97%, at least 98%, at least 99%, at least 99.5%, or 100% identical to SEQ ID NO: 166 or 165. In another example, the FIX coding sequence is (or comprises a sequence) at least 99%, at least 99.5%, or 100% identical to SEQ ID NO: 166 or 165. In another example, the FIX coding sequence comprises the sequence set forth in SEQ ID NO: 166 or 165. In another example, the FIX coding sequence consists essentially of the sequence set forth in SEQ ID NO: 166 or 165. In another example, the FIX coding sequence consists of the sequence set forth in SEQ ID NO: 166 or 165. The FIX coding sequence can be, for example, CpG-depleted (e.g., fully CpG-depleted) and/or codon optimized. For example, the FIX coding sequence can be CpG depleted (e.g., fully CpG-depleted) and codon optimized. Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, the FIX coding sequence in the above examples encodes a FIX protein comprising the sequence set forth in SEQ ID NO: 195. Optionally, the FIX coding sequence in the above examples encodes a FIX protein consisting essentially of the sequence set forth in SEQ ID NO: 195. Optionally, the FIX coding sequence in the above examples encodes a FIX protein consisting of the sequence set forth in SEQ ID NO: 195.

In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 166. In another example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 166 and encodes a FIX protein (or a FIX protein comprising a sequence) at least 99%, at least 99.5%, or 100% identical to SEQ ID NO: 195. In another example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 166 and encodes a FIX protein comprising the sequence set forth in SEQ ID NO: 195. In another example, the FIX coding sequence is (or comprises a sequence) at least 95%, at least 96%, at least 97%, at least 98%, at least 99%, at least 99.5%, or 100% identical to SEQ ID NO: 166. In another example, the FIX coding sequence is (or comprises a sequence) at least 95%, at least 96%, at least 97%, at least 98%, at least 99%, at least 99.5%, or 100% identical to SEQ ID NO: 166 and encodes a FIX protein (or a FIX protein comprising a sequence) at least 99%, at least 99.5%, or 100% identical to SEQ ID NO: 195. In another example, the FIX coding sequence is (or comprises a sequence) at least 95%, at least 96%, at least 97%, at least 98%, at least 99%, at least 99.5%, or 100% identical to SEQ ID NO: 166 and encodes a FIX protein comprising the sequence set forth in SEQ ID NO: 195. In another example, the FIX coding sequence is (or comprises a sequence) at least 99%, at least 99.5%, or 100% identical to SEQ ID NO: 166. In another example, the FIX coding sequence is (or comprises a sequence) at least 99%, at least 99.5%, or 100% identical to SEQ ID NO: 166 and encodes a FIX protein (or a FIX protein comprising a sequence) at least 99%, at least 99.5%, or 100% identical to SEQ ID NO: 195. In another example, the FIX coding sequence is (or comprises a sequence) at least 99%, at least 99.5%, or 100% identical to SEQ ID NO: 166 and encodes a FIX protein comprising the sequence set forth in SEQ ID NO: 195. In another example, the FIX coding sequence comprises the sequence set forth in SEQ ID NO: 166. In another example, the FIX coding sequence consists essentially of the sequence set forth in SEQ ID NO: 166. In another example, the FIX coding sequence consists of the sequence set forth in SEQ ID NO: 166. The FIX coding sequence can be, for example, CpG-depleted (e.g., fully CpG-depleted) and/or codon optimized. For example, the FIX coding sequence can be CpG depleted (e.g., fully CpG-depleted) and codon optimized. Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, the FIX coding sequence in the above examples encodes a FIX protein comprising the sequence set forth in SEQ ID NO: 195. Optionally, the FIX coding sequence in the above examples encodes a FIX protein consisting essentially of the sequence set forth in SEQ ID NO: 195. Optionally, the FIX coding sequence in the above examples encodes a FIX protein consisting of the sequence set forth in SEQ ID NO: 195.

In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 165. In another example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 165 and encodes a FIX protein (or a FIX protein comprising a sequence) at least 99%, at least 99.5%, or 100% identical to SEQ ID NO: 195. In another example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 165 and encodes a FIX protein comprising the sequence set forth in SEQ ID NO: 195. In another example, the FIX coding sequence is (or comprises a sequence) at least 95%, at least 96%, at least 97%, at least 98%, at least 99%, at least 99.5%, or 100% identical to SEQ ID NO: 165. In another example, the FIX coding sequence is (or comprises a sequence) at least 95%, at least 96%, at least 97%, at least 98%, at least 99%, at least 99.5%, or 100% identical to SEQ ID NO: 165 and encodes a FIX protein (or a FIX protein comprising a sequence) at least 99%, at least 99.5%, or 100% identical to SEQ ID NO: 195. In another example, the FIX coding sequence is (or comprises a sequence) at least 95%, at least 96%, at least 97%, at least 98%, at least 99%, at least 99.5%, or 100% identical to SEQ ID NO: 165 and encodes a FIX protein comprising the sequence set forth in SEQ ID NO: 195. In another example, the FIX coding sequence is (or comprises a sequence) at least 99%, at least 99.5%, or 100% identical to SEQ ID NO: 165. In another example, the FIX coding sequence is (or comprises a sequence) at least 99%, at least 99.5%, or 100% identical to SEQ ID NO: 165 and encodes a FIX protein (or a FIX protein comprising a sequence) at least 99%, at least 99.5%, or 100% identical to SEQ ID NO: 195. In another example, the FIX coding sequence is (or comprises a sequence) at least 99%, at least 99.5%, or 100% identical to SEQ ID NO: 165 and encodes a FIX protein comprising the sequence set forth in SEQ ID NO: 195. In another example, the FIX coding sequence comprises the sequence set forth in SEQ ID NO: 165. In another example, the FIX coding sequence consists essentially of the sequence set forth in SEQ ID NO: 165. In another example, the FIX coding sequence consists of the sequence set forth in SEQ ID NO: 165. The FIX coding sequence can be, for example, CpG-depleted (e.g., fully CpG-depleted) and/or codon optimized. For example, the FIX coding sequence can be CpG depleted (e.g., fully CpG-depleted) and codon optimized. Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, the FIX coding sequence in the above examples encodes a FIX protein comprising the sequence set forth in SEQ ID NO: 195. Optionally, the FIX coding sequence in the above examples encodes a FIX protein consisting essentially of the sequence set forth in SEQ ID NO: 195. Optionally, the FIX coding sequence in the above examples encodes a FIX protein consisting of the sequence set forth in SEQ ID NO: 195.

Various optimized native FIX coding sequences are also provided. In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 159-161. In another example, the FIX coding sequence is (or comprises a sequence) at least 95%, at least 96%, at least 97%, at least 98%, at least 99%, at least 99.5%, or 100% identical to any one of SEQ ID NOS: 159-161. In another example, the FIX coding sequence is (or comprises a sequence) at least 99%, at least 99.5%, or 100% identical to any one of SEQ ID NOS: 159-161. In another example, the FIX coding sequence comprises the sequence set forth in any one of SEQ ID NOS: 159-161. In another example, the FIX coding sequence consists essentially of the sequence set forth in any one of SEQ ID NOS: 159-161. In another example, the FIX coding sequence consists of the sequence set forth in any one of SEQ ID NOS: 159-161. The FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the FIX coding sequence can be CpG depleted (e.g., all but one CpG dinucleotides removed) and modified to mutate one or more cryptic splice donor sequences (e.g., all identified cryptic splice donor sequences). Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, the FIX coding sequence in the above examples encodes a FIX protein comprising the sequence set forth in SEQ ID NO: 195. Optionally, the FIX coding sequence in the above examples encodes a FIX protein consisting essentially of the sequence set forth in SEQ ID NO: 195. Optionally, the FIX coding sequence in the above examples encodes a FIX protein consisting of the sequence set forth in SEQ ID NO: 195.

In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159. In another example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the FIX coding sequence is (or comprises a sequence) at least 95%, at least 96%, at least 97%, at least 98%, at least 99%, at least 99.5%, or 100% identical to SEQ ID NO: 159. In another example, the FIX coding sequence is (or comprises a sequence) at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the FIX coding sequence is (or comprises a sequence) at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the FIX coding sequence is (or comprises a sequence) at least 99%, at least 99.5%, or 100% identical to SEQ ID NO: 159. In another example, the FIX coding sequence is (or comprises a sequence) at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the FIX coding sequence is (or comprises a sequence) at least 99%, at least 99.5%, or 100% identical to SEQ ID NO: 159 and encodes a FIX protein comprising the sequence set forth in SEQ ID NO: 195. In another example, the FIX coding sequence comprises the sequence set forth in SEQ ID NO: 159. In another example, the FIX coding sequence consists essentially of the sequence set forth in SEQ ID NO: 159. In another example, the FIX coding sequence consists of the sequence set forth in SEQ ID NO: 159. The FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the FIX coding sequence can be CpG depleted (e.g., all but one CpG dinucleotides removed) and modified to mutate one or more cryptic splice donor sequences (e.g., all identified cryptic splice donor sequences). Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, the FIX coding sequence in the above examples encodes a FIX protein comprising the sequence set forth in SEQ ID NO: 195. Optionally, the FIX coding sequence in the above examples encodes a FIX protein consisting essentially of the sequence set forth in SEQ ID NO: 195. Optionally, the FIX coding sequence in the above examples encodes a FIX protein consisting of the sequence set forth in SEQ ID NO: 195.

When specific F9 nucleic acid constructs sequences are disclosed herein, they are meant to encompass the sequence disclosed or the reverse complement of the sequence. For example, if a F9 nucleic acid construct disclosed herein consists of the hypothetical sequence 5′-CTGGACCGA-3′, it is also meant to encompass the reverse complement of that sequence (5′-TCGGTCCAG-3′). Likewise, when bidirectional construct elements are disclosed herein in a specific 5′ to 3′ order, they are also meant to encompass the reverse complement of the order of those elements. Likewise, when unidirectional construct elements are disclosed herein in a specific 5′ to 3′ order, they are also meant to encompass the reverse complement of the order of those elements. One reason for this is that, in many embodiments disclosed herein, the F9 nucleic acid constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

(2) Bidirectional Constructs

The F9 nucleic acid constructs disclosed herein can be bidirectional constructs. Such bidirectional constructs can allow for enhanced insertion and expression of encoded FIX. When used in combination with a nuclease agent (e.g., CRISPR/Cas system, zinc finger nuclease (ZFN) system; transcription activator-like effector nuclease (TALEN) system) as described herein, the bidirectionality of the nucleic acid construct allows the construct to be inserted in either direction (i.e., is not limited to insertion in one direction) within a target genomic locus, allowing the expression of FIX when inserted in either orientation, thereby enhancing expression efficiency, as exemplified herein. For example, when used in combination with a nuclease agent (e.g., CRISPR/Cas system, zinc finger nuclease (ZFN) system; transcription activator-like effector nuclease (TALEN) system) as described herein, the bidirectionality of the nucleic acid construct allows the construct to be inserted in either direction (i.e., is not limited to insertion in one direction) within a cleavage site or target insertion site, allowing the expression of FIX when inserted in either orientation, thereby enhancing insertion and expression efficiency, as exemplified herein.

A bidirectional construct as disclosed herein can comprise at least two nucleic acid segments, wherein a first segment comprises a first FIX coding sequence, and a second segment comprises the reverse complement of a second FIX coding sequence, or vice versa. However, other bidirectional constructs disclosed herein can comprise at least two nucleic acid segments, wherein the first segment comprises a FIX coding sequence, and the second segment comprises the reverse complement of a coding sequence for another protein, or vice versa. A reverse complement refers to a sequence that is a complement sequence of a reference sequence, wherein the complement sequence is written in the reverse orientation. For example, for a hypothetical sequence 5′-CTGGACCGA-3′, the perfect complement sequence is 3′-GACCTGGCT-5′, and the perfect reverse complement is written 5′-TCGGTCCAG-3′. A reverse complement sequence need not be perfect and may still encode the same polypeptide or a similar polypeptide as the reference sequence. Due to codon usage redundancy, a reverse complement can diverge from a reference sequence that encodes the same polypeptide. The coding sequences can optionally comprise one or more additional sequences, such as sequences encoding amino- or carboxy-terminal amino acid sequences such as a signal sequence, label sequence (e.g., HiBit), or heterologous functional sequence (e.g., nuclear localization sequence (NLS) or self-cleaving peptide) linked to the FIX or other protein.

When specific bidirectional construct sequences are disclosed herein, they are meant to encompass the sequence disclosed or the reverse complement of the sequence. For example, if a bidirectional construct disclosed herein consists of the hypothetical sequence 5′-CTGGACCGA-3′, it is also meant to encompass the reverse complement of that sequence (5′-TCGGTCCAG-3′). Likewise, when bidirectional construct elements are disclosed herein in a specific 5′ to 3′ order, they are also meant to encompass the reverse complement of the order of those elements. For example, if a bidirectional construct is disclosed herein that comprises from 5′ to 3′ a first splice acceptor, a first coding sequence, a first terminator, a reverse complement of a second terminator, a reverse complement of a second coding sequence, and a reverse complement of a second splice acceptor, it is also meant to encompass a construct comprising from 5′ to 3′ the second splice acceptor, the second coding sequence, the second terminator, a reverse complement of the first terminator, a reverse complement of the first coding sequence, and a reverse complement of the first splice acceptor. One reason for this is that, in many embodiments disclosed herein, the bidirectional constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

When the at least two segments both encode FIX, the at least two segments can encode the same FIX protein or different FIX proteins. The different FIX proteins can be at least about 90%, at least about 95%, at least about 96%, at least about 97%, at least about 98%, at least about 99%, or at least about 99.5% identical. For example, the first segment can encode a wild type FIX protein or fragment thereof, and the second segment can encode a variant FIX protein or fragment thereof, or vice versa. Alternatively, the first segment can encode a first variant FIX protein, and the second segment can encode a second variant FIX protein that is different from the first variant FIX protein. Preferably, the two segments encode the same FIX protein (i.e., 100% identical).

Even when the two segments encode the same FIX protein, the FIX coding sequence in the first segment can differ from the FIX coding sequence in the second segment. In some bidirectional constructs, the codon usage in the first coding sequence is the same as the codon usage in the second coding sequence. In other bidirectional constructs, the second coding sequence adopts a different codon usage from the codon usage of the first coding sequence in order to reduce hairpin formation. One or both of the coding sequences can be codon-optimized for expression in a host cell. In some bidirectional constructs, only one of the coding sequences is codon-optimized. In some bidirectional constructs, the first coding sequence is codon-optimized. In some bidirectional constructs, the second coding sequence is codon-optimized. In some bidirectional constructs, both coding sequences are codon-optimized. For example, the second FIX coding sequence can be codon optimized or may use one or more alternative codons for one or more amino acids of the same FIX (i.e., same amino acid sequence) encoded by the FIX coding sequence in the first segment. An alternative codon as used herein refers to variations in codon usage for a given amino acid, and may or may not be a preferred or optimized codon (codon optimized) for a given expression system. Preferred codon usage, or codons that are well-tolerated in a given system of expression are known.

In one example, the second segment comprises a reverse complement of a FIX coding sequence that adopts different codon usage from that of the FIX coding sequence in the first segment in order to reduce hairpin formation. Such a reverse complement forms base pairs with fewer than all nucleotides of the coding sequence in the first segment, yet it optionally encodes the same polypeptide. In one example, the reverse complement sequence in the second segment is not substantially complementary (e.g., not more than 70% complementary) to the coding sequence in the first segment. In other cases, however, the second segment comprises a reverse complement sequence that is highly complementary (e.g., at least 90% complementary) to the coding sequence in the first segment.

The second segment can have any percentage of complementarity to the first segment. For example, the second segment sequence can have at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 97%, or at least about 99% complementarity to the first segment. As another example, the second segment sequence can have less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, less than about 95%, less than about 97%, or less than about 99% complementarity to the first segment. The reverse complement of the second coding sequence can be, in some nucleic acid constructs, not substantially complementary (e.g., not more than 70% complementary) to the first coding sequence, not substantially complementary to a fragment of the first coding sequence, highly complementary (e.g., at least 90% complementary) to the first coding sequence, highly complementary to a fragment of the first coding sequence, about 50% to about 80% identical to the reverse complement of the first coding sequence, or about 60% to about 100% identical to the reverse complement of the first coding sequence.

The bidirectional constructs disclosed herein can be modified to include any suitable structural feature as needed for any particular use and/or that confers one or more desired function. For example, the bidirectional nucleic acid constructs disclosed herein need not comprise a homology arm and/or can be, for example, homology-independent donor constructs. Owing in part to the bidirectional function of the nucleic acid constructs, the bidirectional constructs can be inserted into a genomic locus in either direction as described herein to allow for efficient insertion and/or expression of FIX.

In some cases, the bidirectional nucleic acid construct does not comprise a promoter that drives the expression of FIX. For example, the expression of FIX can be driven by a promoter of the host cell (e.g., the endogenous ALB promoter when the transgene is integrated into a host cell's ALB locus). In other cases, the bidirectional nucleic acid construct can comprise one or more promoters operably linked to the FIX coding sequences. That is, although not required for expression, the constructs disclosed herein may also include transcriptional or translational regulatory sequences such as promoters, enhancers, insulators, internal ribosome entry sites, additional sequences encoding peptides, and/or polyadenylation signals. Some bidirectional constructs can comprise a promoter that drives expression of the first FIX coding sequence and/or the reverse complement of a promoter that drives expression of the reverse complement of the second FIX coding sequence.

The bidirectional constructs disclosed herein can be modified to include or exclude any suitable structural feature as needed for any particular use and/or that confers one or more desired functions. For example, some bidirectional nucleic acid constructs disclosed herein do not comprise a homology arm. Owing in part to the bidirectional function of the nucleic acid construct, the bidirectional construct can be inserted into a genomic locus in either direction (orientation) as described herein to allow for efficient insertion and/or expression of a heterologous FIX.

The bidirectional constructs can, in some cases, comprise one or more (e.g., two) polyadenylation tail sequences or polyadenylation signal sequences. In some bidirectional constructs, the first segment can comprise a polyadenylation signal sequence. In some bidirectional constructs, the second segment can comprise a polyadenylation signal sequence. In some bidirectional constructs, the first segment can comprise a first polyadenylation signal sequence, and the second segment can comprise a second polyadenylation signal sequence (e.g., a reverse complement of a polyadenylation signal sequence). In some bidirectional constructs, the first segment can comprise a first polyadenylation signal sequence located 3′ of the first coding sequence. In some bidirectional constructs, the second segment can comprise a reverse complement of a second polyadenylation signal sequence located 5′ of the reverse complement of the second coding sequence. In some bidirectional constructs, the first segment can comprise a first polyadenylation signal sequence located 3′ of the first coding sequence, and the second segment can comprise a reverse complement of a second polyadenylation signal sequence located 5′ of the reverse complement of the second coding sequence. The first and second polyadenylation signal sequences can be the same or different. In one example, the first and second polyadenylation signals are different. In a specific example, the first polyadenylation signal is a simian virus 40 (SV40) late polyadenylation signal (or a variant thereof), and the second polyadenylation signal is a bovine growth hormone (BGH) polyadenylation signal (or a variant thereof), or vice versa. For example, one polyadenylation signal can be an SV40 polyadenylation signal, and the other polyadenylation signal can be a CpG-depleted BGH polyadenylation signal. In a specific example, one polyadenylation signal can comprise, consist essentially of, or consist of SEQ ID NO: 199, and the other polyadenylation signal can comprise, consist essentially of, or consist of SEQ ID NO: 200.

In some bidirectional constructs, both the first segment and the second segment comprise a polyadenylation tail sequence. Methods of designing a suitable polyadenylation tail sequence are known. For example, in some bidirectional constructs, one or both of the first and second segment comprises a polyadenylation tail sequence and/or a polyadenylation signal sequence downstream of an open reading frame (i.e., a polyadenylation tail sequence and/or a polyadenylation signal sequence 3′ of a coding sequence, or a reverse complement of a polyadenylation tail sequence and/or a polyadenylation signal sequence 5′ of a reverse complement of a coding sequence). The polyadenylation tail sequence can be encoded, for example, as a “poly-A” stretch downstream of the FIX coding sequence (or other protein coding sequence) in the first and/or second segment. A poly-A tail can comprise, for example, at least 20, 30, 40, 50, 60, 70, 80, 90, or 100 adenines, and optionally up to 300 adenines. In a specific example, the poly-A tail comprises 95, 96, 97, 98, 99, or 100 adenine nucleotides. Methods of designing a suitable polyadenylation tail sequence and/or polyadenylation signal sequence are well known. For example, the polyadenylation signal sequence AAUAAA is commonly used in mammalian systems, although variants such as UAUAAA or AU/GUAAA have been identified. See, e.g., Proudfoot (2011) Genes & Dev. 25(17):1770-82, herein incorporated by reference in its entirety for all purposes. In some bidirectional constructs, a single bidirectional terminator can be used to terminate RNA polymerase transcription in either the sense or the antisense direction (i.e., to terminate RNA polymerase transcription from both the first segment and the second segment). Examples of bidirectional terminators include the ARO4, TRP1, TRP4, ADH1, CYC1, GAL1, GALT, and GAL10 terminators.

The bidirectional constructs can, in some cases, comprise one or more (e.g., two) splice acceptor sites. In some bidirectional constructs, the first segment can comprise a splice acceptor site. In some bidirectional constructs, the second segment can comprise a splice acceptor site. In some bidirectional constructs, the first segment can comprise a first splice acceptor site, and the second segment can comprise a second splice acceptor site (e.g., a reverse complement of a splice acceptor site). In some bidirectional constructs, the first segment comprises a first splice acceptor site located 5′ of the first coding sequence. In some bidirectional constructs, the second segment comprises a reverse complement of a second splice acceptor site located 3′ of the reverse complement of the second coding sequence. In some bidirectional constructs, the first segment comprises a first splice acceptor site located 5′ of the first coding sequence, and the second segment comprises a reverse complement of a second splice acceptor site located 3′ of the reverse complement of the second coding sequence. The first and second splice acceptor sites can be the same or different. In a specific example, both splice acceptors are mouse Alb exon 2 splice acceptors. In a specific example, both splice acceptors can comprise, consist essentially of, or consist of SEQ ID NO: 201.

A bidirectional construct may comprise a first coding sequence that encodes a first coding sequence linked to a splice acceptor and a reverse complement of a second coding sequence operably linked to the reverse complement of a splice acceptor. The bidirectional constructs disclosed herein can also comprise a splice acceptor site on either or both ends of the construct, or splice acceptor sites in both the first segment and the second segment (e.g., a splice acceptor site 5′ of a coding sequence, or a reverse complement of a splice acceptor 3′ of a reverse complement of a coding sequence). The splice acceptor site can, for example, comprise NAG or consist of NAG. In a specific example, the splice acceptor is an ALB splice acceptor (e.g., an ALB splice acceptor used in the splicing together of exons 1 and 2 of ALB (i.e., ALB exon 2 splice acceptor)). For example, such a splice acceptor can be derived from the human ALB gene. In another example, the splice acceptor can be derived from the mouse Alb gene (e.g., an ALB splice acceptor used in the splicing together of exons 1 and 2 of mouse Alb (i.e., mouse Alb exon 2 splice acceptor)). In another example, the splice acceptor is a F9 splice acceptor (e.g., the F9 splice acceptor used in the splicing together of exons 1 and 2 of F9). For example, such a splice acceptor can be derived from the human F9 gene. Alternatively, such a splice acceptor can be derived from the mouse F9 gene. Additional suitable splice acceptor sites useful in eukaryotes, including artificial splice acceptors, are known. See, e.g., Shapiro et al. (1987) Nucleic Acids Res. 15:7155-7174 and Burset et al. (2001) Nucleic Acids Res. 29:255-259, each of which is herein incorporated by reference in its entirety for all purposes. The splice acceptors used in a bidirectional construct may be the same or different. In a specific example, both splice acceptors are mouse Alb exon 2 splice acceptors.

The bidirectional constructs can be circular or linear. For example, a bidirectional construct can be linear. The first and second segments can be joined in a linear manner through a linker sequence. For example, the 5′ end of the second segment that comprises a reverse complement sequence can be linked to the 3′ end of the first segment. Alternatively, the 5′ end of the first segment can be linked to the 3′ end of the second segment that comprises a reverse complement sequence. The linker can be any suitable length. For example, the linker can be between about 5 to about 2000 nucleotides in length. As an example, the linker sequence can be about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, about 150, about 200, about 250, about 300, about 500, about 1000, about 1500, about 2000, or more nucleotides in length. Other structural elements in addition to, or instead of, a linker sequence, can also be inserted between the first and second segments.

The bidirectional constructs disclosed herein can be DNA or RNA, single-stranded, double-stranded, or partially single-stranded and partially double-stranded. For example, the constructs can be single- or double-stranded DNA. In some embodiments, the nucleic acid can be modified (e.g., using nucleoside analogs), as described herein. In a specific example, the bidirectional construct is single-stranded (e.g., single-stranded DNA).

The bidirectional constructs disclosed herein can be modified on either or both ends to include one or more suitable structural features as needed and/or to confer one or more functional benefit. For example, structural modifications can vary depending on the method(s) used to deliver the constructs disclosed herein to a host cell (e.g., use of viral vector delivery or packaging into lipid nanoparticles for delivery). Such modifications include, for example, terminal structures such as inverted terminal repeats (ITR), hairpin, loops, and other structures such as toroids. For example, the constructs disclosed herein can comprise one, two, or three ITRs or can comprise no more than two ITRs. Various methods of structural modifications are known.

Similarly, one or both ends of the construct can be protected (e.g., from exonucleolytic degradation) by known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in its entirety for all purposes. Additional methods for protecting the constructs from degradation include, but are not limited to, addition of terminal amino group(s) and the use of modified internucleotide linkages such as, for example, phosphorothioates, phosphoramidates, and O-methyl ribose or deoxyribose residues.

As disclosed in more detail herein, the bidirectional constructs disclosed herein can be introduced into a cell as part of a vector having additional sequences such as, for example, replication origins, promoters, and genes encoding antibiotic resistance. The constructs can be introduced as a naked nucleic acid, can be introduced as a nucleic acid complexed with an agent such as a liposome, polymer, or poloxamer, or can be delivered by viral vectors (e.g., adenovirus, AAV, herpesvirus, retrovirus, lentivirus).

The FIX coding sequences in the bidirectional constructs disclosed herein may include one or more modifications such as codon optimization (e.g., to human codons), depletion of CpG dinucleotides, mutation of cryptic splice sites, addition of one or more glycosylation sites, or any combination thereof. CpG dinucleotides in a construct can limit the therapeutic utility of the construct. First, unmethylated CpG dinucleotides can interact with host toll-like receptor-9 (TLR-9) to stimulate innate, proinflammatory immune responses. Second, once the CpG dinucleotides become methylated, they can result in the suppression of transgene expression coordinated by methyl-CpG binding proteins. Cryptic splice sites are sequences in a pre-messenger RNA that are not normally used as splice sites, but that can be activated, for example, by mutations that either inactivate canonical splice sites or create splice sites where one did not exist before. Accurate splice site selection is critical for successful gene expression, and removal of cryptic splice sites can favor use of the normal or intended splice site.

In one example, a FIX coding sequence in a bidirectional construct disclosed herein has one or more cryptic splice sites mutated or removed. In another example, a FIX coding sequence in a bidirectional construct disclosed herein has all identified cryptic splice sites mutated or removed. In another example, a FIX coding sequence in a bidirectional construct disclosed herein has one or more CpG dinucleotides removed (i.e., is CpG depleted). In another example, a FIX coding sequence in a bidirectional construct disclosed herein has all but one CpG dinucleotides removed. In another example, a FIX coding sequence in a bidirectional construct disclosed herein has all CpG dinucleotides removed (i.e., is fully CpG depleted). In another example, a FIX coding sequence in a bidirectional construct disclosed herein is codon optimized (e.g., codon optimized for expression in a human or mammal). In a specific example, a FIX coding sequence in a bidirectional construct disclosed herein has one or more CpG dinucleotides removed (i.e., is CpG depleted) and has one or more cryptic splice sites mutated or removed. In another specific example, a FIX coding sequence in a bidirectional construct disclosed herein has all but one CpG dinucleotides removed and has one or more or all identified cryptic splice sites mutated or removed. In another specific example, a FIX coding sequence in a bidirectional construct disclosed herein has one or more CpG dinucleotides removed (i.e., is CpG depleted) and is codon optimized (e.g., codon optimized for expression in a human or mammal). In another specific example, a FIX coding sequence in a bidirectional construct disclosed herein has all CpG dinucleotides removed (i.e., is fully CpG depleted) and is codon optimized (e.g., codon optimized for expression in a human or mammal).

In one specific example, one FIX coding sequence in a bidirectional construct disclosed herein has one or more CpG dinucleotides removed (i.e., is CpG depleted) and has one or more cryptic splice sites mutated or removed, and the other FIX coding sequence in the bidirectional construct disclosed herein has one or more CpG dinucleotides removed (i.e., is CpG depleted) and is codon optimized (e.g., codon optimized for expression in a human or mammal). In another specific example, one FIX coding sequence in a bidirectional construct disclosed herein has all but one CpG dinucleotides removed and has one or more or all identified cryptic splice sites mutated or removed, and the other FIX coding sequence in the bidirectional construct disclosed herein has all CpG dinucleotides removed (i.e., is fully CpG depleted) and is codon optimized (e.g., codon optimized for expression in a human or mammal).

In an exemplary bidirectional construct, the second segment is located 3′ of the first segment, the first FIX coding sequence and the second FIX coding sequence both encode the same human FIX protein, the second FIX coding sequence adopts a different codon usage from the codon usage of the first FIX coding sequence, the first segment comprises a first polyadenylation signal sequence located 3′ of the first FIX coding sequence, the second segment comprises a reverse complement of a second polyadenylation signal sequence located 5′ of the reverse complement of the second FIX coding sequence, the first segment comprises a first splice acceptor site located 5′ of the first FIX coding sequence, the second segment comprises a reverse complement of a second splice acceptor site located 3′ of the reverse complement of the second FIX coding sequence, the nucleic acid construct does not comprise a promoter that drives expression of the first FIX protein or the second FIX protein, and optionally the nucleic acid construct does not comprise a homology arm.

In one example of a bidirectional construct, the first FIX protein coding sequence and the second FIX protein coding sequence are different but encode the same FIX protein sequence, and one of the FIX coding sequences is CpG-depleted (e.g., fully CpG-depleted) and/or codon optimized (e.g., CpG-depleted and codon optimized or fully CpG-depleted and codon optimized). In one example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 162-171. In another example, the one of the FIX coding sequences is (or comprises a sequence) at least 95%, at least 96%, at least 97%, at least 98%, at least 99%, at least 99.5%, or 100% identical to any one of SEQ ID NOS: 162-171. In another example, the one of the FIX coding sequences is (or comprises a sequence) at least 99%, at least 99.5%, or 100% identical to any one of SEQ ID NOS: 162-171. In another example, the one of the FIX coding sequences comprises the sequence set forth in any one of SEQ ID NOS: 162-171. In another example, the one of the FIX coding sequences consists essentially of the sequence set forth in any one of SEQ ID NOS: 162-171. In another example, the one of the FIX coding sequences consists of the sequence set forth in any one of SEQ ID NOS: 162-171. Optionally, the one of the FIX coding sequences encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, the one of the FIX coding sequences encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, the one of the FIX coding sequences in the above examples encodes a FIX protein (or a FIX protein comprising a sequence) at least 99%, at least 99.5%, or 100% identical to SEQ ID NO: 195 (and, e.g., retaining the activity of native FIX). Optionally, the one of the FIX coding sequences in the above examples encodes a FIX protein comprising the sequence set forth in SEQ ID NO: 195. Optionally, the one of the FIX coding sequences in the above examples encodes a FIX protein consisting essentially of the sequence set forth in SEQ ID NO: 195. Optionally, the one of the FIX coding sequences in the above examples encodes a FIX protein consisting of the sequence set forth in SEQ ID NO: 195.

In one example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 164-171. In another example, the one of the FIX coding sequences is (or comprises a sequence) at least 95%, at least 96%, at least 97%, at least 98%, at least 99%, at least 99.5%, or 100% identical to any one of SEQ ID NOS: 164-171. In another example, the one of the FIX coding sequences is (or comprises a sequence) at least 99%, at least 99.5%, or 100% identical to any one of SEQ ID NOS: 164-171. In another example, the one of the FIX coding sequences comprises the sequence set forth in any one of SEQ ID NOS: 164-171. In another example, the one of the FIX coding sequences consists essentially of the sequence set forth in any one of SEQ ID NOS: 164-171. In another example, the one of the FIX coding sequences consists of the sequence set forth in any one of SEQ ID NOS: 164-171. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. Optionally, the one of the FIX coding sequences encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, the one of the FIX coding sequences encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, the one of the FIX coding sequences in the above examples encodes a FIX protein at least 99%, at least 99.5%, or 100% identical to SEQ ID NO: 195 (and, e.g., retaining the activity of native FIX). Optionally, the one of the FIX coding sequences in the above examples encodes a FIX protein (or a FIX protein comprising a sequence) comprising the sequence set forth in SEQ ID NO: 195. Optionally, the one of the FIX coding sequences in the above examples encodes a FIX protein consisting essentially of the sequence set forth in SEQ ID NO: 195. Optionally, the one of the FIX coding sequences in the above examples encodes a FIX protein consisting of the sequence set forth in SEQ ID NO: 195.

In one example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 166 or 165. In another example, the one of the FIX coding sequences is (or comprises a sequence) at least 95%, at least 96%, at least 97%, at least 98%, at least 99%, at least 99.5%, or 100% identical to SEQ ID NO: 166 or 165. In another example, the one of the FIX coding sequences is (or comprises a sequence) at least 99%, at least 99.5%, or 100% identical to SEQ ID NO: 166 or 165. In another example, the one of the FIX coding sequences comprises the sequence set forth in SEQ ID NO: 166 or 165. In another example, the one of the FIX coding sequences consists essentially of the sequence set forth in SEQ ID NO: 166 or 165. In another example, the one of the FIX coding sequences consists of the sequence set forth in SEQ ID NO: 166 or 165. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. Optionally, the one of the FIX coding sequences encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, the one of the FIX coding sequences encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, the one of the FIX coding sequences in the above examples encodes a FIX protein (or a FIX protein comprising a sequence) at least 99%, at least 99.5%, or 100% identical to SEQ ID NO: 195 (and, e.g., retaining the activity of native FIX). Optionally, the one of the FIX coding sequences in the above examples encodes a FIX protein comprising the sequence set forth in SEQ ID NO: 195. Optionally, the one of the FIX coding sequences in the above examples encodes a FIX protein consisting essentially of the sequence set forth in SEQ ID NO: 195. Optionally, the one of the FIX coding sequences in the above examples encodes a FIX protein consisting of the sequence set forth in SEQ ID NO: 195.

In one example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 166. In another example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 166 and encodes a FIX protein (or a FIX protein comprising a sequence) at least 99%, at least 99.5%, or 100% identical to SEQ ID NO: 195. In another example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 166 and encodes a FIX protein comprising the sequence set forth in SEQ ID NO: 195. In another example, the one of the FIX coding sequences is (or comprises a sequence) at least 95%, at least 96%, at least 97%, at least 98%, at least 99%, at least 99.5%, or 100% identical to SEQ ID NO: 166. In another example, the one of the FIX coding sequences is (or comprises a sequence) at least 95%, at least 96%, at least 97%, at least 98%, at least 99%, at least 99.5%, or 100% identical to SEQ ID NO: 166 and encodes a FIX protein (or a FIX protein comprising a sequence) at least 99%, at least 99.5%, or 100% identical to SEQ ID NO: 195. In another example, the one of the FIX coding sequences is (or comprises a sequence) at least 95%, at least 96%, at least 97%, at least 98%, at least 99%, at least 99.5%, or 100% identical to SEQ ID NO: 166 and encodes a FIX protein comprising the sequence set forth in SEQ ID NO: 195. In another example, the one of the FIX coding sequences is (or comprises a sequence) at least 99%, at least 99.5%, or 100% identical to SEQ ID NO: 166. In another example, the one of the FIX coding sequences is (or comprises a sequence) at least 99%, at least 99.5%, or 100% identical to SEQ ID NO: 166 and encodes a FIX protein (or a FIX protein comprising a sequence) at least 99%, at least 99.5%, or 100% identical to SEQ ID NO: 195. In another example, the one of the FIX coding sequences is (or comprises a sequence) at least 99%, at least 99.5%, or 100% identical to SEQ ID NO: 166 and encodes a FIX protein comprising the sequence set forth in SEQ ID NO: 195. In another example, the one of the FIX coding sequences comprises the sequence set forth in SEQ ID NO: 166. In another example, the one of the FIX coding sequences consists essentially of the sequence set forth in SEQ ID NO: 166. In another example, the one of the FIX coding sequences consists of the sequence set forth in SEQ ID NO: 166. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. Optionally, the one of the FIX coding sequences encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, the one of the FIX coding sequences encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, the one of the FIX coding sequences in the above examples encodes a FIX protein (or a FIX protein comprising a sequence) at least 99%, at least 99.5%, or 100% identical to SEQ ID NO: 195 (and, e.g., retaining the activity of native FIX). Optionally, the one of the FIX coding sequences in the above examples encodes a FIX protein comprising the sequence set forth in SEQ ID NO: 195. Optionally, the one of the FIX coding sequences in the above examples encodes a FIX protein consisting essentially of the sequence set forth in SEQ ID NO: 195. Optionally, the one of the FIX coding sequences in the above examples encodes a FIX protein consisting of the sequence set forth in SEQ ID NO: 195.

In one example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 165. In another example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 165 and encodes a FIX protein (or a FIX protein comprising a sequence) at least 99%, at least 99.5%, or 100% identical to SEQ ID NO: 195. In another example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 165 and encodes a FIX protein comprising the sequence set forth in SEQ ID NO: 195. In another example, the one of the FIX coding sequences is (or comprises a sequence) at least 95%, at least 96%, at least 97%, at least 98%, at least 99%, at least 99.5%, or 100% identical to SEQ ID NO: 165. In another example, the one of the FIX coding sequences is (or comprises a sequence) at least 95%, at least 96%, at least 97%, at least 98%, at least 99%, at least 99.5%, or 100% identical to SEQ ID NO: 165 and encodes a FIX protein (or a FIX protein comprising a sequence) at least 99%, at least 99.5%, or 100% identical to SEQ ID NO: 195. In another example, the one of the FIX coding sequences is (or comprises a sequence) at least 95%, at least 96%, at least 97%, at least 98%, at least 99%, at least 99.5%, or 100% identical to SEQ ID NO: 165 and encodes a FIX protein comprising the sequence set forth in SEQ ID NO: 195. In another example, the one of the FIX coding sequences is (or comprises a sequence) at least 99%, at least 99.5%, or 100% identical to SEQ ID NO: 165. In another example, the one of the FIX coding sequences is (or comprises a sequence) at least 99%, at least 99.5%, or 100% identical to SEQ ID NO: 165 and encodes a FIX protein (or a FIX protein comprising a sequence) at least 99%, at least 99.5%, or 100% identical to SEQ ID NO: 195. In another example, the one of the FIX coding sequences is (or comprises a sequence) at least 99%, at least 99.5%, or 100% identical to SEQ ID NO: 165 and encodes a FIX protein comprising the sequence set forth in SEQ ID NO: 195. In another example, the one of the FIX coding sequences comprises the sequence set forth in SEQ ID NO: 165. In another example, the one of the FIX coding sequences consists essentially of the sequence set forth in SEQ ID NO: 165. In another example, the one of the FIX coding sequences consists of the sequence set forth in SEQ ID NO: 165. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. Optionally, the one of the FIX coding sequences encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, the one of the FIX coding sequences encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, the one of the FIX coding sequences in the above examples encodes a FIX protein (or a FIX protein comprising a sequence) at least 99%, at least 99.5%, or 100% identical to SEQ ID NO: 195 (and, e.g., retaining the activity of native FIX). Optionally, the one of the FIX coding sequences in the above examples encodes a FIX protein comprising the sequence set forth in SEQ ID NO: 195. Optionally, the one of the FIX coding sequences in the above examples encodes a FIX protein consisting essentially of the sequence set forth in SEQ ID NO: 195. Optionally, the one of the FIX coding sequences in the above examples encodes a FIX protein consisting of the sequence set forth in SEQ ID NO: 195.

Various optimized native FIX coding sequences are also provided. In one example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 159-161. In another example, the one of the FIX coding sequences is (or comprises a sequence) at least 95%, at least 96%, at least 97%, at least 98%, at least 99%, at least 99.5%, or 100% identical to any one of SEQ ID NOS: 159-161. In another example, the one of the FIX coding sequences is (or comprises a sequence) at least 99%, at least 99.5%, or 100% identical to any one of SEQ ID NOS: 159-161. In another example, the one of the FIX coding sequences comprises the sequence set forth in any one of SEQ ID NOS: 159-161. In another example, the one of the FIX coding sequences consists essentially of the sequence set forth in any one of SEQ ID NOS: 159-161. In another example, the one of the FIX coding sequences consists of the sequence set forth in any one of SEQ ID NOS: 159-161. The one of the FIX coding sequences can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the one of the FIX coding sequences can be CpG depleted (e.g., all but one CpG dinucleotides removed or fully CpG-depleted) and modified to mutate one or more cryptic splice donor sequences (e.g., all identified cryptic splice donor sequences). Optionally, the one of the FIX coding sequences encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, the one of the FIX coding sequences encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, the one of the FIX coding sequences in the above examples encodes a FIX protein (or a FIX protein comprising a sequence) at least 99%, at least 99.5%, or 100% identical to SEQ ID NO: 195 (and, e.g., retaining the activity of native FIX). Optionally, the one of the FIX coding sequences in the above examples encodes a FIX protein comprising the sequence set forth in SEQ ID NO: 195. Optionally, the one of the FIX coding sequences in the above examples encodes a FIX protein consisting essentially of the sequence set forth in SEQ ID NO: 195. Optionally, the one of the FIX coding sequences in the above examples encodes a FIX protein consisting of the sequence set forth in SEQ ID NO: 195.

In one example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159. In another example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the one of the FIX coding sequences is (or comprises a sequence) at least 95%, at least 96%, at least 97%, at least 98%, at least 99%, at least 99.5%, or 100% identical to SEQ ID NO: 159. In another example, the one of the FIX coding sequences is (or comprises a sequence) at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the one of the FIX coding sequences is (or comprises a sequence) at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the one of the FIX coding sequences is (or comprises a sequence) at least 99%, at least 99.5%, or 100% identical to SEQ ID NO: 159. In another example, the one of the FIX coding sequences is (or comprises a sequence) at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the one of the FIX coding sequences is (or comprises a sequence) at least 99%, at least 99.5%, or 100% identical to SEQ ID NO: 159 and encodes a FIX protein comprising the sequence set forth in SEQ ID NO: 195. In another example, the one of the FIX coding sequences comprises the sequence set forth in SEQ ID NO: 159. In another example, the one of the FIX coding sequences consists essentially of the sequence set forth in SEQ ID NO: 159. In another example, the one of the FIX coding sequences consists of the sequence set forth in SEQ ID NO: 159. The one of the FIX coding sequences can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the one of the FIX coding sequences can be CpG depleted (e.g., all but one CpG dinucleotides removed or fully CpG-depleted) and modified to mutate one or more cryptic splice donor sequences (e.g., all identified cryptic splice donor sequences). Optionally, the one of the FIX coding sequences encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, the one of the FIX coding sequences encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, the one of the FIX coding sequences in the above examples encodes a FIX protein (or a FIX protein comprising a sequence) at least 99%, at least 99.5%, or 100% identical to SEQ ID NO: 195 (and, e.g., retaining the activity of native FIX). Optionally, the one of the FIX coding sequences in the above examples encodes a FIX protein comprising the sequence set forth in SEQ ID NO: 195. Optionally, the FIX coding sequence in the above examples encodes a FIX protein consisting essentially of the sequence set forth in SEQ ID NO: 195. Optionally, the one of the FIX coding sequences in the above examples encodes a FIX protein consisting of the sequence set forth in SEQ ID NO: 195.

In one example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 166. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 99%, at least 99.5%, or 100% identical to SEQ ID NO: 159. In another example, the other FIX coding sequence is (or comprises a sequence) at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 99%, at least 99.5%, or 100% identical to SEQ ID NO: 159 and encodes a FIX protein comprising the sequence set forth in SEQ ID NO: 195. In another example, the other FIX coding sequence comprises the sequence set forth in SEQ ID NO: 159. In another example, the other FIX coding sequence consists essentially of the sequence set forth in SEQ ID NO: 159. In another example, the other FIX coding sequence consists of the sequence set forth in SEQ ID NO: 159. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, one or both of the FIX coding sequence in the above examples encodes a FIX protein comprising the sequence set forth in SEQ ID NO: 195. Optionally, one or both of the FIX coding sequence in the above examples encodes a FIX protein consisting essentially of the sequence set forth in SEQ ID NO: 195. Optionally, one or both of the FIX coding sequence in the above examples encodes a FIX protein consisting of the sequence set forth in SEQ ID NO: 195.

In another example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 166 and encodes a FIX protein (or a FIX protein comprising a sequence) at least 99%, at least 99.5%, or 100% identical to SEQ ID NO: 195. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 99%, at least 99.5%, or 100% identical to SEQ ID NO: 159. In another example, the other FIX coding sequence is (or comprises a sequence) at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 99%, at least 99.5%, or 100% identical to SEQ ID NO: 159 and encodes a FIX protein comprising the sequence set forth in SEQ ID NO: 195. In another example, the other FIX coding sequence comprises the sequence set forth in SEQ ID NO: 159. In another example, the other FIX coding sequence consists essentially of the sequence set forth in SEQ ID NO: 159. In another example, the other FIX coding sequence consists of the sequence set forth in SEQ ID NO: 159. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, one or both of the FIX coding sequence in the above examples encodes a FIX protein comprising the sequence set forth in SEQ ID NO: 195. Optionally, one or both of the FIX coding sequence in the above examples encodes a FIX protein consisting essentially of the sequence set forth in SEQ ID NO: 195. Optionally, one or both of the FIX coding sequence in the above examples encodes a FIX protein consisting of the sequence set forth in SEQ ID NO: 195.

In another example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 166 and encodes a FIX protein comprising the sequence set forth in SEQ ID NO: 195. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 99%, at least 99.5%, or 100% identical to SEQ ID NO: 159. In another example, the other FIX coding sequence is (or comprises a sequence) at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 99%, at least 99.5%, or 100% identical to SEQ ID NO: 159 and encodes a FIX protein comprising the sequence set forth in SEQ ID NO: 195. In another example, the other FIX coding sequence comprises the sequence set forth in SEQ ID NO: 159. In another example, the other FIX coding sequence consists essentially of the sequence set forth in SEQ ID NO: 159. In another example, the other FIX coding sequence consists of the sequence set forth in SEQ ID NO: 159. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, one or both of the FIX coding sequence in the above examples encodes a FIX protein comprising the sequence set forth in SEQ ID NO: 195. Optionally, one or both of the FIX coding sequence in the above examples encodes a FIX protein consisting essentially of the sequence set forth in SEQ ID NO: 195. Optionally, one or both of the FIX coding sequence in the above examples encodes a FIX protein consisting of the sequence set forth in SEQ ID NO: 195.

In another example, the one of the FIX coding sequences is (or comprises a sequence) at least 95%, at least 96%, at least 97%, at least 98%, at least 99%, at least 99.5%, or 100% identical to SEQ ID NO: 166. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 99%, at least 99.5%, or 100% identical to SEQ ID NO: 159. In another example, the other FIX coding sequence is (or comprises a sequence) at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 99%, at least 99.5%, or 100% identical to SEQ ID NO: 159 and encodes a FIX protein comprising the sequence set forth in SEQ ID NO: 195. In another example, the other FIX coding sequence comprises the sequence set forth in SEQ ID NO: 159. In another example, the other FIX coding sequence consists essentially of the sequence set forth in SEQ ID NO: 159. In another example, the other FIX coding sequence consists of the sequence set forth in SEQ ID NO: 159. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, one or both of the FIX coding sequence in the above examples encodes a FIX protein comprising the sequence set forth in SEQ ID NO: 195. Optionally, one or both of the FIX coding sequence in the above examples encodes a FIX protein consisting essentially of the sequence set forth in SEQ ID NO: 195. Optionally, one or both of the FIX coding sequence in the above examples encodes a FIX protein consisting of the sequence set forth in SEQ ID NO: 195.

In another example, the one of the FIX coding sequences is (or comprises a sequence) at least 95%, at least 96%, at least 97%, at least 98%, at least 99%, at least 99.5%, or 100% identical to SEQ ID NO: 166 and encodes a FIX protein (or a FIX protein comprising a sequence) at least 99%, at least 99.5%, or 100% identical to SEQ ID NO: 195. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 99%, at least 99.5%, or 100% identical to SEQ ID NO: 159. In another example, the other FIX coding sequence is (or comprises a sequence) at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 99%, at least 99.5%, or 100% identical to SEQ ID NO: 159 and encodes a FIX protein comprising the sequence set forth in SEQ ID NO: 195. In another example, the other FIX coding sequence comprises the sequence set forth in SEQ ID NO: 159. In another example, the other FIX coding sequence consists essentially of the sequence set forth in SEQ ID NO: 159. In another example, the other FIX coding sequence consists of the sequence set forth in SEQ ID NO: 159. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, one or both of the FIX coding sequence in the above examples encodes a FIX protein comprising the sequence set forth in SEQ ID NO: 195. Optionally, one or both of the FIX coding sequence in the above examples encodes a FIX protein consisting essentially of the sequence set forth in SEQ ID NO: 195. Optionally, one or both of the FIX coding sequence in the above examples encodes a FIX protein consisting of the sequence set forth in SEQ ID NO: 195.

In another example, the one of the FIX coding sequences is (or comprises a sequence) at least 95%, at least 96%, at least 97%, at least 98%, at least 99%, at least 99.5%, or 100% identical to SEQ ID NO: 166 and encodes a FIX protein comprising the sequence set forth in SEQ ID NO: 195. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 99%, at least 99.5%, or 100% identical to SEQ ID NO: 159. In another example, the other FIX coding sequence is (or comprises a sequence) at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 99%, at least 99.5%, or 100% identical to SEQ ID NO: 159 and encodes a FIX protein comprising the sequence set forth in SEQ ID NO: 195. In another example, the other FIX coding sequence comprises the sequence set forth in SEQ ID NO: 159. In another example, the other FIX coding sequence consists essentially of the sequence set forth in SEQ ID NO: 159. In another example, the other FIX coding sequence consists of the sequence set forth in SEQ ID NO: 159. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, one or both of the FIX coding sequence in the above examples encodes a FIX protein comprising the sequence set forth in SEQ ID NO: 195. Optionally, one or both of the FIX coding sequence in the above examples encodes a FIX protein consisting essentially of the sequence set forth in SEQ ID NO: 195. Optionally, one or both of the FIX coding sequence in the above examples encodes a FIX protein consisting of the sequence set forth in SEQ ID NO: 195.

In another example, the one of the FIX coding sequences is (or comprises a sequence) at least 99%, at least 99.5%, or 100% identical to SEQ ID NO: 166. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 99%, at least 99.5%, or 100% identical to SEQ ID NO: 159. In another example, the other FIX coding sequence is (or comprises a sequence) at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 99%, at least 99.5%, or 100% identical to SEQ ID NO: 159 and encodes a FIX protein comprising the sequence set forth in SEQ ID NO: 195. In another example, the other FIX coding sequence comprises the sequence set forth in SEQ ID NO: 159. In another example, the other FIX coding sequence consists essentially of the sequence set forth in SEQ ID NO: 159. In another example, the other FIX coding sequence consists of the sequence set forth in SEQ ID NO: 159. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, one or both of the FIX coding sequence in the above examples encodes a FIX protein comprising the sequence set forth in SEQ ID NO: 195. Optionally, one or both of the FIX coding sequence in the above examples encodes a FIX protein consisting essentially of the sequence set forth in SEQ ID NO: 195. Optionally, one or both of the FIX coding sequence in the above examples encodes a FIX protein consisting of the sequence set forth in SEQ ID NO: 195.

In another example, the one of the FIX coding sequences is (or comprises a sequence) at least 99%, at least 99.5%, or 100% identical to SEQ ID NO: 166 and encodes a FIX protein (or a FIX protein comprising a sequence) at least 99%, at least 99.5%, or 100% identical to SEQ ID NO: 195. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 99%, at least 99.5%, or 100% identical to SEQ ID NO: 159. In another example, the other FIX coding sequence is (or comprises a sequence) at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 99%, at least 99.5%, or 100% identical to SEQ ID NO: 159 and encodes a FIX protein comprising the sequence set forth in SEQ ID NO: 195. In another example, the other FIX coding sequence comprises the sequence set forth in SEQ ID NO: 159. In another example, the other FIX coding sequence consists essentially of the sequence set forth in SEQ ID NO: 159. In another example, the other FIX coding sequence consists of the sequence set forth in SEQ ID NO: 159. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, one or both of the FIX coding sequence in the above examples encodes a FIX protein comprising the sequence set forth in SEQ ID NO: 195. Optionally, one or both of the FIX coding sequence in the above examples encodes a FIX protein consisting essentially of the sequence set forth in SEQ ID NO: 195. Optionally, one or both of the FIX coding sequence in the above examples encodes a FIX protein consisting of the sequence set forth in SEQ ID NO: 195.

In another example, the one of the FIX coding sequences is (or comprises a sequence) at least 99%, at least 99.5%, or 100% identical to SEQ ID NO: 166 and encodes a FIX protein comprising the sequence set forth in SEQ ID NO: 195. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 99%, at least 99.5%, or 100% identical to SEQ ID NO: 159. In another example, the other FIX coding sequence is (or comprises a sequence) at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 99%, at least 99.5%, or 100% identical to SEQ ID NO: 159 and encodes a FIX protein comprising the sequence set forth in SEQ ID NO: 195. In another example, the other FIX coding sequence comprises the sequence set forth in SEQ ID NO: 159. In another example, the other FIX coding sequence consists essentially of the sequence set forth in SEQ ID NO: 159. In another example, the other FIX coding sequence consists of the sequence set forth in SEQ ID NO: 159. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, one or both of the FIX coding sequence in the above examples encodes a FIX protein comprising the sequence set forth in SEQ ID NO: 195. Optionally, one or both of the FIX coding sequence in the above examples encodes a FIX protein consisting essentially of the sequence set forth in SEQ ID NO: 195. Optionally, one or both of the FIX coding sequence in the above examples encodes a FIX protein consisting of the sequence set forth in SEQ ID NO: 195.

In another example, the one of the FIX coding sequences comprises the sequence set forth in SEQ ID NO: 166. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 99%, at least 99.5%, or 100% identical to SEQ ID NO: 159. In another example, the other FIX coding sequence is (or comprises a sequence) at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 99%, at least 99.5%, or 100% identical to SEQ ID NO: 159 and encodes a FIX protein comprising the sequence set forth in SEQ ID NO: 195. In another example, the other FIX coding sequence comprises the sequence set forth in SEQ ID NO: 159. In another example, the other FIX coding sequence consists essentially of the sequence set forth in SEQ ID NO: 159. In another example, the other FIX coding sequence consists of the sequence set forth in SEQ ID NO: 159. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, one or both of the FIX coding sequence in the above examples encodes a FIX protein comprising the sequence set forth in SEQ ID NO: 195. Optionally, one or both of the FIX coding sequence in the above examples encodes a FIX protein consisting essentially of the sequence set forth in SEQ ID NO: 195. Optionally, one or both of the FIX coding sequence in the above examples encodes a FIX protein consisting of the sequence set forth in SEQ ID NO: 195.

In another example, the one of the FIX coding sequences consists essentially of the sequence set forth in SEQ ID NO: 166. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 99%, at least 99.5%, or 100% identical to SEQ ID NO: 159. In another example, the other FIX coding sequence is (or comprises a sequence) at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 99%, at least 99.5%, or 100% identical to SEQ ID NO: 159 and encodes a FIX protein comprising the sequence set forth in SEQ ID NO: 195. In another example, the other FIX coding sequence comprises the sequence set forth in SEQ ID NO: 159. In another example, the other FIX coding sequence consists essentially of the sequence set forth in SEQ ID NO: 159. In another example, the other FIX coding sequence consists of the sequence set forth in SEQ ID NO: 159. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, one or both of the FIX coding sequence in the above examples encodes a FIX protein comprising the sequence set forth in SEQ ID NO: 195. Optionally, one or both of the FIX coding sequence in the above examples encodes a FIX protein consisting essentially of the sequence set forth in SEQ ID NO: 195. Optionally, one or both of the FIX coding sequence in the above examples encodes a FIX protein consisting of the sequence set forth in SEQ ID NO: 195.

In another example, the one of the FIX coding sequences consists of the sequence set forth in SEQ ID NO: 166. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 99%, at least 99.5%, or 100% identical to SEQ ID NO: 159. In another example, the other FIX coding sequence is (or comprises a sequence) at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 99%, at least 99.5%, or 100% identical to SEQ ID NO: 159 and encodes a FIX protein comprising the sequence set forth in SEQ ID NO: 195. In another example, the other FIX coding sequence comprises the sequence set forth in SEQ ID NO: 159. In another example, the other FIX coding sequence consists essentially of the sequence set forth in SEQ ID NO: 159. In another example, the other FIX coding sequence consists of the sequence set forth in SEQ ID NO: 159. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, one or both of the FIX coding sequence in the above examples encodes a FIX protein comprising the sequence set forth in SEQ ID NO: 195. Optionally, one or both of the FIX coding sequence in the above examples encodes a FIX protein consisting essentially of the sequence set forth in SEQ ID NO: 195. Optionally, one or both of the FIX coding sequence in the above examples encodes a FIX protein consisting of the sequence set forth in SEQ ID NO: 195.

In one example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 165. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 99%, at least 99.5%, or 100% identical to SEQ ID NO: 159. In another example, the other FIX coding sequence is (or comprises a sequence) at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 99%, at least 99.5%, or 100% identical to SEQ ID NO: 159 and encodes a FIX protein comprising the sequence set forth in SEQ ID NO: 195. In another example, the other FIX coding sequence comprises the sequence set forth in SEQ ID NO: 159. In another example, the other FIX coding sequence consists essentially of the sequence set forth in SEQ ID NO: 159. In another example, the other FIX coding sequence consists of the sequence set forth in SEQ ID NO: 159. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, one or both of the FIX coding sequence in the above examples encodes a FIX protein comprising the sequence set forth in SEQ ID NO: 195. Optionally, one or both of the FIX coding sequence in the above examples encodes a FIX protein consisting essentially of the sequence set forth in SEQ ID NO: 195. Optionally, one or both of the FIX coding sequence in the above examples encodes a FIX protein consisting of the sequence set forth in SEQ ID NO: 195.

In another example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 165 and encodes a FIX protein (or a FIX protein comprising a sequence) at least 99%, at least 99.5%, or 100% identical to SEQ ID NO: 195. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 99%, at least 99.5%, or 100% identical to SEQ ID NO: 159. In another example, the other FIX coding sequence is (or comprises a sequence) at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 99%, at least 99.5%, or 100% identical to SEQ ID NO: 159 and encodes a FIX protein comprising the sequence set forth in SEQ ID NO: 195. In another example, the other FIX coding sequence comprises the sequence set forth in SEQ ID NO: 159. In another example, the other FIX coding sequence consists essentially of the sequence set forth in SEQ ID NO: 159. In another example, the other FIX coding sequence consists of the sequence set forth in SEQ ID NO: 159. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, one or both of the FIX coding sequence in the above examples encodes a FIX protein comprising the sequence set forth in SEQ ID NO: 195. Optionally, one or both of the FIX coding sequence in the above examples encodes a FIX protein consisting essentially of the sequence set forth in SEQ ID NO: 195. Optionally, one or both of the FIX coding sequence in the above examples encodes a FIX protein consisting of the sequence set forth in SEQ ID NO: 195.

In another example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 165 and encodes a FIX protein comprising the sequence set forth in SEQ ID NO: 195. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 99%, at least 99.5%, or 100% identical to SEQ ID NO: 159. In another example, the other FIX coding sequence is (or comprises a sequence) at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 99%, at least 99.5%, or 100% identical to SEQ ID NO: 159 and encodes a FIX protein comprising the sequence set forth in SEQ ID NO: 195. In another example, the other FIX coding sequence comprises the sequence set forth in SEQ ID NO: 159. In another example, the other FIX coding sequence consists essentially of the sequence set forth in SEQ ID NO: 159. In another example, the other FIX coding sequence consists of the sequence set forth in SEQ ID NO: 159. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, one or both of the FIX coding sequence in the above examples encodes a FIX protein comprising the sequence set forth in SEQ ID NO: 195. Optionally, one or both of the FIX coding sequence in the above examples encodes a FIX protein consisting essentially of the sequence set forth in SEQ ID NO: 195. Optionally, one or both of the FIX coding sequence in the above examples encodes a FIX protein consisting of the sequence set forth in SEQ ID NO: 195.

In another example, the one of the FIX coding sequences is (or comprises a sequence) at least 95%, at least 96%, at least 97%, at least 98%, at least 99%, at least 99.5%, or 100% identical to SEQ ID NO: 165. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 99%, at least 99.5%, or 100% identical to SEQ ID NO: 159. In another example, the other FIX coding sequence is (or comprises a sequence) at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 99%, at least 99.5%, or 100% identical to SEQ ID NO: 159 and encodes a FIX protein comprising the sequence set forth in SEQ ID NO: 195. In another example, the other FIX coding sequence comprises the sequence set forth in SEQ ID NO: 159. In another example, the other FIX coding sequence consists essentially of the sequence set forth in SEQ ID NO: 159. In another example, the other FIX coding sequence consists of the sequence set forth in SEQ ID NO: 159. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, one or both of the FIX coding sequence in the above examples encodes a FIX protein comprising the sequence set forth in SEQ ID NO: 195. Optionally, one or both of the FIX coding sequence in the above examples encodes a FIX protein consisting essentially of the sequence set forth in SEQ ID NO: 195. Optionally, one or both of the FIX coding sequence in the above examples encodes a FIX protein consisting of the sequence set forth in SEQ ID NO: 195.

In another example, the one of the FIX coding sequences is (or comprises a sequence) at least 95%, at least 96%, at least 97%, at least 98%, at least 99%, at least 99.5%, or 100% identical to SEQ ID NO: 165 and encodes a FIX protein (or a FIX protein comprising a sequence) at least 99%, at least 99.5%, or 100% identical to SEQ ID NO: 195. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 99%, at least 99.5%, or 100% identical to SEQ ID NO: 159. In another example, the other FIX coding sequence is (or comprises a sequence) at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 99%, at least 99.5%, or 100% identical to SEQ ID NO: 159 and encodes a FIX protein comprising the sequence set forth in SEQ ID NO: 195. In another example, the other FIX coding sequence comprises the sequence set forth in SEQ ID NO: 159. In another example, the other FIX coding sequence consists essentially of the sequence set forth in SEQ ID NO: 159. In another example, the other FIX coding sequence consists of the sequence set forth in SEQ ID NO: 159. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, one or both of the FIX coding sequence in the above examples encodes a FIX protein comprising the sequence set forth in SEQ ID NO: 195. Optionally, one or both of the FIX coding sequence in the above examples encodes a FIX protein consisting essentially of the sequence set forth in SEQ ID NO: 195. Optionally, one or both of the FIX coding sequence in the above examples encodes a FIX protein consisting of the sequence set forth in SEQ ID NO: 195.

In another example, the one of the FIX coding sequences is (or comprises a sequence) at least 95%, at least 96%, at least 97%, at least 98%, at least 99%, at least 99.5%, or 100% identical to SEQ ID NO: 165 and encodes a FIX protein comprising the sequence set forth in SEQ ID NO: 195. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 99%, at least 99.5%, or 100% identical to SEQ ID NO: 159. In another example, the other FIX coding sequence is (or comprises a sequence) at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 99%, at least 99.5%, or 100% identical to SEQ ID NO: 159 and encodes a FIX protein comprising the sequence set forth in SEQ ID NO: 195. In another example, the other FIX coding sequence comprises the sequence set forth in SEQ ID NO: 159. In another example, the other FIX coding sequence consists essentially of the sequence set forth in SEQ ID NO: 159. In another example, the other FIX coding sequence consists of the sequence set forth in SEQ ID NO: 159. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, one or both of the FIX coding sequence in the above examples encodes a FIX protein comprising the sequence set forth in SEQ ID NO: 195. Optionally, one or both of the FIX coding sequence in the above examples encodes a FIX protein consisting essentially of the sequence set forth in SEQ ID NO: 195. Optionally, one or both of the FIX coding sequence in the above examples encodes a FIX protein consisting of the sequence set forth in SEQ ID NO: 195.

In another example, the one of the FIX coding sequences is (or comprises a sequence) at least 99%, at least 99.5%, or 100% identical to SEQ ID NO: 165. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 99%, at least 99.5%, or 100% identical to SEQ ID NO: 159. In another example, the other FIX coding sequence is (or comprises a sequence) at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 99%, at least 99.5%, or 100% identical to SEQ ID NO: 159 and encodes a FIX protein comprising the sequence set forth in SEQ ID NO: 195. In another example, the other FIX coding sequence comprises the sequence set forth in SEQ ID NO: 159. In another example, the other FIX coding sequence consists essentially of the sequence set forth in SEQ ID NO: 159. In another example, the other FIX coding sequence consists of the sequence set forth in SEQ ID NO: 159. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, one or both of the FIX coding sequence in the above examples encodes a FIX protein comprising the sequence set forth in SEQ ID NO: 195. Optionally, one or both of the FIX coding sequence in the above examples encodes a FIX protein consisting essentially of the sequence set forth in SEQ ID NO: 195. Optionally, one or both of the FIX coding sequence in the above examples encodes a FIX protein consisting of the sequence set forth in SEQ ID NO: 195.

In another example, the one of the FIX coding sequences is (or comprises a sequence) at least 99%, at least 99.5%, or 100% identical to SEQ ID NO: 165 and encodes a FIX protein (or a FIX protein comprising a sequence) at least 99%, at least 99.5%, or 100% identical to SEQ ID NO: 195. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 99%, at least 99.5%, or 100% identical to SEQ ID NO: 159. In another example, the other FIX coding sequence is (or comprises a sequence) at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 99%, at least 99.5%, or 100% identical to SEQ ID NO: 159 and encodes a FIX protein comprising the sequence set forth in SEQ ID NO: 195. In another example, the other FIX coding sequence comprises the sequence set forth in SEQ ID NO: 159. In another example, the other FIX coding sequence consists essentially of the sequence set forth in SEQ ID NO: 159. In another example, the other FIX coding sequence consists of the sequence set forth in SEQ ID NO: 159. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, one or both of the FIX coding sequence in the above examples encodes a FIX protein comprising the sequence set forth in SEQ ID NO: 195. Optionally, one or both of the FIX coding sequence in the above examples encodes a FIX protein consisting essentially of the sequence set forth in SEQ ID NO: 195. Optionally, one or both of the FIX coding sequence in the above examples encodes a FIX protein consisting of the sequence set forth in SEQ ID NO: 195.

In another example, the one of the FIX coding sequences is (or comprises a sequence) at least 99%, at least 99.5%, or 100% identical to SEQ ID NO: 165 and encodes a FIX protein comprising the sequence set forth in SEQ ID NO: 195. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 99%, at least 99.5%, or 100% identical to SEQ ID NO: 159. In another example, the other FIX coding sequence is (or comprises a sequence) at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 99%, at least 99.5%, or 100% identical to SEQ ID NO: 159 and encodes a FIX protein comprising the sequence set forth in SEQ ID NO: 195. In another example, the other FIX coding sequence comprises the sequence set forth in SEQ ID NO: 159. In another example, the other FIX coding sequence consists essentially of the sequence set forth in SEQ ID NO: 159. In another example, the other FIX coding sequence consists of the sequence set forth in SEQ ID NO: 159. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, one or both of the FIX coding sequence in the above examples encodes a FIX protein comprising the sequence set forth in SEQ ID NO: 195. Optionally, one or both of the FIX coding sequence in the above examples encodes a FIX protein consisting essentially of the sequence set forth in SEQ ID NO: 195. Optionally, one or both of the FIX coding sequence in the above examples encodes a FIX protein consisting of the sequence set forth in SEQ ID NO: 195.

In another example, the one of the FIX coding sequences comprises the sequence set forth in SEQ ID NO: 165. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 99%, at least 99.5%, or 100% identical to SEQ ID NO: 159. In another example, the other FIX coding sequence is (or comprises a sequence) at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 99%, at least 99.5%, or 100% identical to SEQ ID NO: 159 and encodes a FIX protein comprising the sequence set forth in SEQ ID NO: 195. In another example, the other FIX coding sequence comprises the sequence set forth in SEQ ID NO: 159. In another example, the other FIX coding sequence consists essentially of the sequence set forth in SEQ ID NO: 159. In another example, the other FIX coding sequence consists of the sequence set forth in SEQ ID NO: 159. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, one or both of the FIX coding sequence in the above examples encodes a FIX protein comprising the sequence set forth in SEQ ID NO: 195. Optionally, one or both of the FIX coding sequence in the above examples encodes a FIX protein consisting essentially of the sequence set forth in SEQ ID NO: 195. Optionally, one or both of the FIX coding sequence in the above examples encodes a FIX protein consisting of the sequence set forth in SEQ ID NO: 195.

In another example, the one of the FIX coding sequences consists essentially of the sequence set forth in SEQ ID NO: 165. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 99%, at least 99.5%, or 100% identical to SEQ ID NO: 159. In another example, the other FIX coding sequence is (or comprises a sequence) at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 99%, at least 99.5%, or 100% identical to SEQ ID NO: 159 and encodes a FIX protein comprising the sequence set forth in SEQ ID NO: 195. In another example, the other FIX coding sequence comprises the sequence set forth in SEQ ID NO: 159. In another example, the other FIX coding sequence consists essentially of the sequence set forth in SEQ ID NO: 159. In another example, the other FIX coding sequence consists of the sequence set forth in SEQ ID NO: 159. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, one or both of the FIX coding sequence in the above examples encodes a FIX protein comprising the sequence set forth in SEQ ID NO: 195. Optionally, one or both of the FIX coding sequence in the above examples encodes a FIX protein consisting essentially of the sequence set forth in SEQ ID NO: 195. Optionally, one or both of the FIX coding sequence in the above examples encodes a FIX protein consisting of the sequence set forth in SEQ ID NO: 195.

In another example, the one of the FIX coding sequences consists of the sequence set forth in SEQ ID NO: 165. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the other FIX coding sequence is (or comprises a sequence) at least 99%, at least 99.5%, or 100% identical to SEQ ID NO: 159. In another example, the other FIX coding sequence is (or comprises a sequence) at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the other FIX coding sequence is (or comprises a sequence) at least 99%, at least 99.5%, or 100% identical to SEQ ID NO: 159 and encodes a FIX protein comprising the sequence set forth in SEQ ID NO: 195. In another example, the other FIX coding sequence comprises the sequence set forth in SEQ ID NO: 159. In another example, the other FIX coding sequence consists essentially of the sequence set forth in SEQ ID NO: 159. In another example, the other FIX coding sequence consists of the sequence set forth in SEQ ID NO: 159. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, one or both of the FIX coding sequence in the above examples encodes a FIX protein comprising the sequence set forth in SEQ ID NO: 195. Optionally, one or both of the FIX coding sequence in the above examples encodes a FIX protein consisting essentially of the sequence set forth in SEQ ID NO: 195. Optionally, one or both of the FIX coding sequence in the above examples encodes a FIX protein consisting of the sequence set forth in SEQ ID NO: 195.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to any one of SEQ ID NOS: 202-224 or 172-194. In another particular example, an exemplary bidirectional construct comprises a sequence at least 95%, at least 96%, at least 97%, at least 98%, at least 99%, at least 99.5%, or 100% identical to any one of SEQ ID NOS: 202-224 or 172-194. In another particular example, an exemplary bidirectional construct comprises a sequence at least 99%, at least 99.5%, or 100% identical to any one of SEQ ID NOS: 202-224 or 172-194. In another particular example, an exemplary bidirectional construct comprises any one of SEQ ID NOS: 202-224 or 172-194. In another particular example, an exemplary bidirectional construct consists essentially of any one of SEQ ID NOS: 202-224 or 172-194. In another particular example, an exemplary bidirectional construct consists of any one of SEQ ID NOS: 202-224 or 172-194.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to any one of SEQ ID NOS: 203-224 or 173-194. In another particular example, an exemplary bidirectional construct comprises a sequence at least 95%, at least 96%, at least 97%, at least 98%, at least 99%, at least 99.5%, or 100% identical to any one of SEQ ID NOS: 203-224 or 173-194. In another particular example, an exemplary bidirectional construct comprises a sequence at least 99%, at least 99.5%, or 100% identical to any one of SEQ ID NOS: 203-224 or 173-194. In another particular example, an exemplary bidirectional construct comprises any one of SEQ ID NOS: 203-224 or 173-194. In another particular example, an exemplary bidirectional construct consists essentially of any one of SEQ ID NOS: 203-224 or 173-194. In another particular example, an exemplary bidirectional construct consists of any one of SEQ ID NOS: 203-224 or 173-194.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to any one of SEQ ID NOS: 204-224 or 174-194. In another particular example, an exemplary bidirectional construct comprises a sequence at least 95%, at least 96%, at least 97%, at least 98%, at least 99%, at least 99.5%, or 100% identical to any one of SEQ ID NOS: 204-224 or 174-194. In another particular example, an exemplary bidirectional construct comprises a sequence at least 99%, at least 99.5%, or 100% identical to any one of SEQ ID NOS: 204-224 or 174-194. In another particular example, an exemplary bidirectional construct comprises any one of SEQ ID NOS: 204-224 or 174-194. In another particular example, an exemplary bidirectional construct consists essentially of any one of SEQ ID NOS: 204-224 or 174-194. In another particular example, an exemplary bidirectional construct consists of any one of SEQ ID NOS: 204-224 or 174-194.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 210 or 209 or 180 or 179. In another particular example, an exemplary bidirectional construct comprises a sequence at least 95%, at least 96%, at least 97%, at least 98%, at least 99%, at least 99.5%, or 100% identical to SEQ ID NO: 210 or 209 or 180 or 179. In another particular example, an exemplary bidirectional construct comprises a sequence at least 99%, at least 99.5%, or 100% identical to SEQ ID NO: 210 or 209 or 180 or 179. In another particular example, an exemplary bidirectional construct comprises SEQ ID NO: 210 or 209 or 180 or 179. In another particular example, an exemplary bidirectional construct consists essentially of SEQ ID NO: 210 or 209 or 180 or 179. In another particular example, an exemplary bidirectional construct consists of SEQ ID NO: 210 or 209 or 180 or 179.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 210 or 180. In another particular example, an exemplary bidirectional construct comprises a sequence at least 95%, at least 96%, at least 97%, at least 98%, at least 99%, at least 99.5%, or 100% identical to SEQ ID NO: 210 or 180. In another particular example, an exemplary bidirectional construct comprises a sequence at least 99%, at least 99.5%, or 100% identical to SEQ ID NO: 210 or 180. In another particular example, an exemplary bidirectional construct comprises SEQ ID NO: 210 or 180. In another particular example, an exemplary bidirectional construct consists essentially of SEQ ID NO: 210 or 180. In another particular example, an exemplary bidirectional construct consists of SEQ ID NO: 210 or 180.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 210. In another particular example, an exemplary bidirectional construct comprises a sequence at least 95%, at least 96%, at least 97%, at least 98%, at least 99%, at least 99.5%, or 100% identical to SEQ ID NO: 210. In another particular example, an exemplary bidirectional construct comprises a sequence at least 99%, at least 99.5%, or 100% identical to SEQ ID NO: 210. In another particular example, an exemplary bidirectional construct comprises SEQ ID NO: 210. In another particular example, an exemplary bidirectional construct consists essentially of SEQ ID NO: 210. In another particular example, an exemplary bidirectional construct consists of SEQ ID NO: 210.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 180. In another particular example, an exemplary bidirectional construct comprises a sequence at least 95%, at least 96%, at least 97%, at least 98%, at least 99%, at least 99.5%, or 100% identical to SEQ ID NO: 180. In another particular example, an exemplary bidirectional construct comprises a sequence at least 99%, at least 99.5%, or 100% identical to SEQ ID NO: 180. In another particular example, an exemplary bidirectional construct comprises SEQ ID NO: 180. In another particular example, an exemplary bidirectional construct consists essentially of SEQ ID NO: 180. In another particular example, an exemplary bidirectional construct consists of SEQ ID NO: 180.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 209 or 179. In another particular example, an exemplary bidirectional construct comprises a sequence at least 95%, at least 96%, at least 97%, at least 98%, at least 99%, at least 99.5%, or 100% identical to SEQ ID NO: 209 or 179. In another particular example, an exemplary bidirectional construct comprises a sequence at least 99%, at least 99.5%, or 100% identical to SEQ ID NO: 209 or 179. In another particular example, an exemplary bidirectional construct comprises SEQ ID NO: 209 or 179. In another particular example, an exemplary bidirectional construct consists essentially of SEQ ID NO: 209 or 179. In another particular example, an exemplary bidirectional construct consists of SEQ ID NO: 209 or 179.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 209. In another particular example, an exemplary bidirectional construct comprises a sequence at least 95%, at least 96%, at least 97%, at least 98%, at least 99%, at least 99.5%, or 100% identical to SEQ ID NO: 209. In another particular example, an exemplary bidirectional construct comprises a sequence at least 99%, at least 99.5%, or 100% identical to SEQ ID NO: 209. In another particular example, an exemplary bidirectional construct comprises SEQ ID NO: 209. In another particular example, an exemplary bidirectional construct consists essentially of SEQ ID NO: 209. In another particular example, an exemplary bidirectional construct consists of SEQ ID NO: 209.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 179. In another particular example, an exemplary bidirectional construct comprises a sequence at least 95%, at least 96%, at least 97%, at least 98%, at least 99%, at least 99.5%, or 100% identical to SEQ ID NO: 179. In another particular example, an exemplary bidirectional construct comprises a sequence at least 99%, at least 99.5%, or 100% identical to SEQ ID NO: 179. In another particular example, an exemplary bidirectional construct comprises SEQ ID NO: 179. In another particular example, an exemplary bidirectional construct consists essentially of SEQ ID NO: 179. In another particular example, an exemplary bidirectional construct consists of SEQ ID NO: 179.

(3) Unidirectional Constructs

The F9 nucleic acid constructs disclosed herein can be unidirectional constructs.

When specific unidirectional construct sequences are disclosed herein, they are meant to encompass the sequence disclosed or the reverse complement of the sequence. For example, if a unidirectional construct disclosed herein consists of the hypothetical sequence 5′-CTGGACCGA-3′, it is also meant to encompass the reverse complement of that sequence (5′-TCGGTCCAG-3′). Likewise, when unidirectional construct elements are disclosed herein in a specific 5′ to 3′ order, they are also meant to encompass the reverse complement of the order of those elements. One reason for this is that, in many embodiments disclosed herein, the unidirectional constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

In the unidirectional constructs, the FIX coding sequence can be a wild type FIX coding sequence without further modification. In the unidirectional constructs, the FIX coding sequence can be codon-optimized for expression in a host cell. For example, the FIX coding sequence can be codon optimized or may use one or more alternative codons for one or more amino acids of the FIX (i.e., same amino acid sequence). An alternative codon as used herein refers to variations in codon usage for a given amino acid, and may or may not be a preferred or optimized codon (codon optimized) for a given expression system. Preferred codon usage, or codons that are well-tolerated in a given system of expression, are known.

The unidirectional constructs disclosed herein can be modified to include any suitable structural feature as needed for any particular use and/or that confers one or more desired functions. For example, the unidirectional nucleic acid constructs disclosed herein need not comprise a homology arm and/or can be, for example, homology-independent donor constructs.

In some cases, the unidirectional nucleic acid construct does not comprise a promoter that drives the expression of FIX. For example, the expression of FIX can be driven by a promoter of the host cell (e.g., the endogenous ALB promoter when the transgene is integrated into a host cell's ALB locus). In other cases, the unidirectional nucleic acid construct can comprise one or more promoters operably linked to the FIX coding sequence. That is, although not required for expression, the constructs disclosed herein may also include transcriptional or translational regulatory sequences such as promoters, enhancers, insulators, internal ribosome entry sites, additional sequences encoding peptides, and/or polyadenylation signals. Some unidirectional constructs can comprise a promoter that drives expression of the FIX coding sequence.

The unidirectional constructs can, in some cases, comprise one or more polyadenylation tail sequences or polyadenylation signal sequences. Some unidirectional constructs can comprise a polyadenylation signal sequence located 3′ of the FIX coding sequence. In a specific example, the polyadenylation signal is a simian virus 40 (SV40) late polyadenylation signal (or a variant thereof). In another specific example, the polyadenylation signal is a bovine growth hormone (BGH) polyadenylation signal (or a variant thereof). In another specific example, the polyadenylation signal is a CpG-depleted BGH polyadenylation signal. For example, the polyadenylation signal can be an SV40 polyadenylation signal or a CpG-depleted BGH polyadenylation signal. In a specific example, the polyadenylation signal can comprise, consist essentially of, or consist of SEQ ID NO: 199. In another specific example, the polyadenylation signal can comprise, consist essentially of, or consist of SEQ ID NO: 200.

Methods of designing a suitable polyadenylation tail sequence are known. For example, some unidirectional constructs comprise a polyadenylation tail sequence and/or a polyadenylation signal sequence downstream of an open reading frame (i.e., a polyadenylation tail sequence and/or a polyadenylation signal sequence 3′ of a coding sequence). The polyadenylation tail sequence can be encoded, for example, as a “poly-A” stretch downstream of the FIX coding sequence (or other protein coding sequence) in the first and/or second segment. A poly-A tail can comprise, for example, at least 20, 30, 40, 50, 60, 70, 80, 90, or 100 adenines, and optionally up to 300 adenines. In a specific example, the poly-A tail comprises 95, 96, 97, 98, 99, or 100 adenine nucleotides. Methods of designing a suitable polyadenylation tail sequence and/or polyadenylation signal sequence are well known. For example, the polyadenylation signal sequence AAUAAA is commonly used in mammalian systems, although variants such as UAUAAA or AU/GUAAA have been identified. See, e.g., Proudfoot (2011) Genes & Dev. 25(17):1770-82, herein incorporated by reference in its entirety for all purposes.

The unidirectional constructs can, in some cases, comprise one or more splice acceptor sites. Some unidirectional constructs comprise a splice acceptor site located 5′ of the FIX coding sequence. In a specific example, the splice acceptor is a mouse Alb exon 2 splice acceptor. In a specific example, the splice acceptor can comprise, consist essentially of, or consist of SEQ ID NO: 201.

The splice acceptor site can, for example, comprise NAG or consist of NAG. In a specific example, the splice acceptor is an ALB splice acceptor (e.g., an ALB splice acceptor used in the splicing together of exons 1 and 2 of ALB (i.e., ALB exon 2 splice acceptor)). For example, such a splice acceptor can be derived from the human ALB gene. In another example, the splice acceptor can be derived from the mouse Alb gene (e.g., an ALB splice acceptor used in the splicing together of exons 1 and 2 of mouse Alb (i.e., mouse Alb exon 2 splice acceptor)). In another example, the splice acceptor is a F9 splice acceptor (e.g., the F9 splice acceptor used in the splicing together of exons 1 and 2 of F9). For example, such a splice acceptor can be derived from the human F9 gene. Alternatively, such a splice acceptor can be derived from the mouse F9 gene. Additional suitable splice acceptor sites useful in eukaryotes, including artificial splice acceptors, are known. See, e.g., Shapiro et al. (1987) Nucleic Acids Res. 15:7155-7174 and Burset et al. (2001) Nucleic Acids Res. 29:255-259, each of which is herein incorporated by reference in its entirety for all purposes.

The unidirectional constructs can be circular or linear. For example, a unidirectional construct can be linear.

The unidirectional constructs disclosed herein can be DNA or RNA, single-stranded, double-stranded, or partially single-stranded and partially double-stranded. For example, the constructs can be single- or double-stranded DNA. In some embodiments, the nucleic acid can be modified (e.g., using nucleoside analogs), as described herein. In a specific example, the unidirectional construct is single-stranded (e.g., single-stranded DNA).

The unidirectional constructs disclosed herein can be modified on either or both ends to include one or more suitable structural features as needed and/or to confer one or more functional benefit. For example, structural modifications can vary depending on the method(s) used to deliver the constructs disclosed herein to a host cell (e.g., use of viral vector delivery or packaging into lipid nanoparticles for delivery). Such modifications include, for example, terminal structures such as inverted terminal repeats (ITR), hairpin, loops, and other structures such as toroids. For example, the constructs disclosed herein can comprise one, two, or three ITRs or can comprise no more than two ITRs. Various methods of structural modifications are known.

Similarly, one or both ends of the construct can be protected (e.g., from exonucleolytic degradation) by known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in its entirety for all purposes. Additional methods for protecting the constructs from degradation include, but are not limited to, addition of terminal amino group(s) and the use of modified internucleotide linkages such as, for example, phosphorothioates, phosphoramidates, and O-methyl ribose or deoxyribose residues.

As disclosed in more detail herein, the unidirectional constructs disclosed herein can be introduced into a cell as part of a vector having additional sequences such as, for example, replication origins, promoters, and genes encoding antibiotic resistance. The constructs can be introduced as a naked nucleic acid, can be introduced as a nucleic acid complexed with an agent such as a liposome, polymer, or poloxamer, or can be delivered by viral vectors (e.g., adenovirus, AAV, herpesvirus, retrovirus, lentivirus).

The FIX coding sequences in the unidirectional constructs disclosed herein may include one or more modifications such as codon optimization (e.g., to human codons), depletion of CpG dinucleotides, mutation of cryptic splice sites, addition of one or more glycosylation sites, or any combination thereof. CpG dinucleotides in a construct can limit the therapeutic utility of the construct. First, unmethylated CpG dinucleotides can interact with host toll-like receptor-9 (TLR-9) to stimulate innate, proinflammatory immune responses. Second, once the CpG dinucleotides become methylated, they can result in the suppression of transgene expression coordinated by methyl-CpG binding proteins. Cryptic splice sites are sequences in a pre-messenger RNA that are not normally used as splice sites, but that can be activated, for example, by mutations that either inactivate canonical splice sites or create splice sites where one did not exist before. Accurate splice site selection is critical for successful gene expression, and removal of cryptic splice sites can favor use of the normal or intended splice site.

In one example, a FIX coding sequence in a unidirectional construct disclosed herein has one or more cryptic splice sites mutated or removed. In another example, a FIX coding sequence in a unidirectional construct disclosed herein has all identified cryptic splice sites mutated or removed. In another example, a FIX coding sequence in a unidirectional construct disclosed herein has one or more CpG dinucleotides removed (i.e., is CpG depleted). In another example, a FIX coding sequence in a unidirectional construct disclosed herein has all but one CpG dinucleotides removed. In another example, a FIX coding sequence in a unidirectional construct disclosed herein has all CpG dinucleotides removed (i.e., is fully CpG depleted). In another example, a FIX coding sequence in a unidirectional construct disclosed herein is codon optimized (e.g., codon optimized for expression in a human or mammal). In a specific example, a FIX coding sequence in a unidirectional construct disclosed herein has one or more CpG dinucleotides removed (i.e., is CpG depleted) and has one or more cryptic splice sites mutated or removed. In another specific example, a FIX coding sequence in a unidirectional construct disclosed herein has all but one CpG dinucleotides removed and has one or more or all identified cryptic splice sites mutated or removed. In another specific example, a FIX coding sequence in a unidirectional construct disclosed herein has one or more CpG dinucleotides removed (i.e., is CpG depleted) and is codon optimized (e.g., codon optimized for expression in a human or mammal). In another specific example, a FIX coding sequence in a unidirectional construct disclosed herein has all CpG dinucleotides removed (i.e., is fully CpG depleted) and is codon optimized (e.g., codon optimized for expression in a human or mammal).

In an exemplary unidirectional construct, the construct comprises a polyadenylation signal sequence located 3′ of the FIX coding sequence, the construct comprises a splice acceptor site located 5′ of the FIX coding sequence, and the nucleic acid construct does not comprise a promoter that drives expression of the FIX protein, and optionally the nucleic acid construct does not comprise a homology arm.

In one example of a unidirectional construct, the FIX protein coding sequence is CpG-depleted (e.g., fully CpG-depleted) and/or codon optimized (e.g., CpG-depleted and codon optimized or fully CpG-depleted and codon optimized). In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 162-171. In another example, the FIX coding sequence is (or comprises a sequence) at least 95%, at least 96%, at least 97%, at least 98%, at least 99%, at least 99.5%, or 100% identical to any one of SEQ ID NOS: 162-171. In another example, the FIX coding sequence is (or comprises a sequence) at least 99%, at least 99.5%, or 100% identical to any one of SEQ ID NOS: 162-171. In another example, the FIX coding sequence comprises the sequence set forth in any one of SEQ ID NOS: 162-171. In another example, the FIX coding sequence consists essentially of the sequence set forth in any one of SEQ ID NOS: 162-171. In another example, the FIX coding sequence consists of the sequence set forth in any one of SEQ ID NOS: 162-171. Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, the FIX coding sequence in the above examples encodes a FIX protein comprising the sequence set forth in SEQ ID NO: 195. Optionally, the FIX coding sequence in the above examples encodes a FIX protein consisting essentially of the sequence set forth in SEQ ID NO: 195. Optionally, the FIX coding sequence in the above examples encodes a FIX protein consisting of the sequence set forth in SEQ ID NO: 195.

In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 164-171. In another example, the FIX coding sequence is (or comprises a sequence) at least 95%, at least 96%, at least 97%, at least 98%, at least 99%, at least 99.5%, or 100% identical to any one of SEQ ID NOS: 164-171. In another example, the FIX coding sequence is (or comprises a sequence) at least 99%, at least 99.5%, or 100% identical to any one of SEQ ID NOS: 164-171. In another example, the FIX coding sequence comprises the sequence set forth in any one of SEQ ID NOS: 164-171. In another example, the FIX coding sequence consists essentially of the sequence set forth in any one of SEQ ID NOS: 164-171. In another example, the FIX coding sequence consists of the sequence set forth in any one of SEQ ID NOS: 164-171. The FIX coding sequence can be, for example, CpG-depleted (e.g., fully CpG-depleted) and/or codon optimized. For example, the FIX coding sequence can be CpG depleted (e.g., fully CpG-depleted) and codon optimized. Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, the FIX coding sequence in the above examples encodes a FIX protein at least 99%, at least 99.5%, or 100% identical to SEQ ID NO: 195 (and, e.g., retaining the activity of native FIX). Optionally, the FIX coding sequence in the above examples encodes a FIX protein (or a FIX protein comprising a sequence) comprising the sequence set forth in SEQ ID NO: 195. Optionally, the FIX coding sequence in the above examples encodes a FIX protein consisting essentially of the sequence set forth in SEQ ID NO: 195. Optionally, the FIX coding sequence in the above examples encodes a FIX protein consisting of the sequence set forth in SEQ ID NO: 195.

In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 166 or 165. In another example, the FIX coding sequence is (or comprises a sequence) at least 95%, at least 96%, at least 97%, at least 98%, at least 99%, at least 99.5%, or 100% identical to SEQ ID NO: 166 or 165. In another example, the FIX coding sequence is (or comprises a sequence) at least 99%, at least 99.5%, or 100% identical to SEQ ID NO: 166 or 165. In another example, the FIX coding sequence comprises the sequence set forth in SEQ ID NO: 166 or 165. In another example, the FIX coding sequence consists essentially of the sequence set forth in SEQ ID NO: 166 or 165. In another example, the FIX coding sequence consists of the sequence set forth in SEQ ID NO: 166 or 165. The FIX coding sequence can be, for example, CpG-depleted (e.g., fully CpG-depleted) and/or codon optimized. For example, the FIX coding sequence can be CpG depleted (e.g., fully CpG-depleted) and codon optimized. Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, the FIX coding sequence in the above examples encodes a FIX protein comprising the sequence set forth in SEQ ID NO: 195. Optionally, the FIX coding sequence in the above examples encodes a FIX protein consisting essentially of the sequence set forth in SEQ ID NO: 195. Optionally, the FIX coding sequence in the above examples encodes a FIX protein consisting of the sequence set forth in SEQ ID NO: 195.

In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 166. In another example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 166 and encodes a FIX protein (or a FIX protein comprising a sequence) at least 99%, at least 99.5%, or 100% identical to SEQ ID NO: 195. In another example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 166 and encodes a FIX protein comprising the sequence set forth in SEQ ID NO: 195. In another example, the FIX coding sequence is (or comprises a sequence) at least 95%, at least 96%, at least 97%, at least 98%, at least 99%, at least 99.5%, or 100% identical to SEQ ID NO: 166. In another example, the FIX coding sequence is (or comprises a sequence) at least 95%, at least 96%, at least 97%, at least 98%, at least 99%, at least 99.5%, or 100% identical to SEQ ID NO: 166 and encodes a FIX protein (or a FIX protein comprising a sequence) at least 99%, at least 99.5%, or 100% identical to SEQ ID NO: 195. In another example, the FIX coding sequence is (or comprises a sequence) at least 95%, at least 96%, at least 97%, at least 98%, at least 99%, at least 99.5%, or 100% identical to SEQ ID NO: 166 and encodes a FIX protein comprising the sequence set forth in SEQ ID NO: 195. In another example, the FIX coding sequence is (or comprises a sequence) at least 99%, at least 99.5%, or 100% identical to SEQ ID NO: 166. In another example, the FIX coding sequence is (or comprises a sequence) at least 99%, at least 99.5%, or 100% identical to SEQ ID NO: 166 and encodes a FIX protein (or a FIX protein comprising a sequence) at least 99%, at least 99.5%, or 100% identical to SEQ ID NO: 195. In another example, the FIX coding sequence is (or comprises a sequence) at least 99%, at least 99.5%, or 100% identical to SEQ ID NO: 166 and encodes a FIX protein comprising the sequence set forth in SEQ ID NO: 195. In another example, the FIX coding sequence comprises the sequence set forth in SEQ ID NO: 166. In another example, the FIX coding sequence consists essentially of the sequence set forth in SEQ ID NO: 166. In another example, the FIX coding sequence consists of the sequence set forth in SEQ ID NO: 166. The FIX coding sequence can be, for example, CpG-depleted (e.g., fully CpG-depleted) and/or codon optimized. For example, the FIX coding sequence can be CpG depleted (e.g., fully CpG-depleted) and codon optimized. Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, the FIX coding sequence in the above examples encodes a FIX protein comprising the sequence set forth in SEQ ID NO: 195. Optionally, the FIX coding sequence in the above examples encodes a FIX protein consisting essentially of the sequence set forth in SEQ ID NO: 195. Optionally, the FIX coding sequence in the above examples encodes a FIX protein consisting of the sequence set forth in SEQ ID NO: 195.

In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 165. In another example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 165 and encodes a FIX protein (or a FIX protein comprising a sequence) at least 99%, at least 99.5%, or 100% identical to SEQ ID NO: 195. In another example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 165 and encodes a FIX protein comprising the sequence set forth in SEQ ID NO: 195. In another example, the FIX coding sequence is (or comprises a sequence) at least 95%, at least 96%, at least 97%, at least 98%, at least 99%, at least 99.5%, or 100% identical to SEQ ID NO: 165. In another example, the FIX coding sequence is (or comprises a sequence) at least 95%, at least 96%, at least 97%, at least 98%, at least 99%, at least 99.5%, or 100% identical to SEQ ID NO: 165 and encodes a FIX protein (or a FIX protein comprising a sequence) at least 99%, at least 99.5%, or 100% identical to SEQ ID NO: 195. In another example, the FIX coding sequence is (or comprises a sequence) at least 95%, at least 96%, at least 97%, at least 98%, at least 99%, at least 99.5%, or 100% identical to SEQ ID NO: 165 and encodes a FIX protein comprising the sequence set forth in SEQ ID NO: 195. In another example, the FIX coding sequence is (or comprises a sequence) at least 99%, at least 99.5%, or 100% identical to SEQ ID NO: 165. In another example, the FIX coding sequence is (or comprises a sequence) at least 99%, at least 99.5%, or 100% identical to SEQ ID NO: 165 and encodes a FIX protein (or a FIX protein comprising a sequence) at least 99%, at least 99.5%, or 100% identical to SEQ ID NO: 195. In another example, the FIX coding sequence is (or comprises a sequence) at least 99%, at least 99.5%, or 100% identical to SEQ ID NO: 165 and encodes a FIX protein comprising the sequence set forth in SEQ ID NO: 195. In another example, the FIX coding sequence comprises the sequence set forth in SEQ ID NO: 165. In another example, the FIX coding sequence consists essentially of the sequence set forth in SEQ ID NO: 165. In another example, the one of the FIX coding sequences consists of the sequence set forth in SEQ ID NO: 165. The FIX coding sequence can be, for example, CpG-depleted (e.g., fully CpG-depleted) and/or codon optimized. For example, the FIX coding sequence can be CpG depleted (e.g., fully CpG-depleted) and codon optimized. Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, the FIX coding sequence in the above examples encodes a FIX protein comprising the sequence set forth in SEQ ID NO: 195. Optionally, the FIX coding sequence in the above examples encodes a FIX protein consisting essentially of the sequence set forth in SEQ ID NO: 195. Optionally, the FIX coding sequence in the above examples encodes a FIX protein consisting of the sequence set forth in SEQ ID NO: 195.

Various optimized native FIX coding sequences are also provided. In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 159-161. In another example, the FIX coding sequence is (or comprises a sequence) at least 95%, at least 96%, at least 97%, at least 98%, at least 99%, at least 99.5%, or 100% identical to any one of SEQ ID NOS: 159-161. In another example, the FIX coding sequence is (or comprises a sequence) at least 99%, at least 99.5%, or 100% identical to any one of SEQ ID NOS: 159-161. In another example, the FIX coding sequence comprises the sequence set forth in any one of SEQ ID NOS: 159-161. In another example, the FIX coding sequence consists essentially of the sequence set forth in any one of SEQ ID NOS: 159-161. In another example, the FIX coding sequence consists of the sequence set forth in any one of SEQ ID NOS: 159-161. The FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, the FIX coding sequence in the above examples encodes a FIX protein comprising the sequence set forth in SEQ ID NO: 195. Optionally, the FIX coding sequence in the above examples encodes a FIX protein consisting essentially of the sequence set forth in SEQ ID NO: 195. Optionally, the FIX coding sequence in the above examples encodes a FIX protein consisting of the sequence set forth in SEQ ID NO: 195.

In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159. In another example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the FIX coding sequence is (or comprises a sequence) at least 95%, at least 96%, at least 97%, at least 98%, at least 99%, at least 99.5%, or 100% identical to SEQ ID NO: 159. In another example, the FIX coding sequence is (or comprises a sequence) at least 95%, at least 96%, at least 97%, at least 98%, at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the FIX coding sequence is (or comprises a sequence) at least 95%, at least 96%, at least 97%, at least 98%, at least 99%, at least 99.5%, or 100% identical to SEQ ID NO: 159 and encodes a FIX protein comprising the sequence set forth in SEQ ID NO: 195. In another example, the FIX coding sequence is (or comprises a sequence) at least 99%, at least 99.5%, or 100% identical to SEQ ID NO: 159. In another example, the FIX coding sequence is (or comprises a sequence) at least 99%, at least 99.5%, or 100% identical to SEQ ID NO: 159 and encodes a FIX protein (or a FIX protein comprising a sequence) at least 99%, at least 99.5%, or 100% identical to SEQ ID NO: 195. In another example, the FIX coding sequence is (or comprises a sequence) at least 99%, at least 99.5%, or 100% identical to SEQ ID NO: 159 and encodes a FIX protein comprising the sequence set forth in SEQ ID NO: 195. In another example, the FIX coding sequence comprises the sequence set forth in SEQ ID NO: 159. In another example, the FIX coding sequence consists essentially of the sequence set forth in SEQ ID NO: 159. In another example, the one of the FIX coding sequences consists of the sequence set forth in SEQ ID NO: 159. The FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 195 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 195 (and, e.g., retaining the activity of native FIX). Optionally, the FIX coding sequence in the above examples encodes a FIX protein (or a FIX protein comprising a sequence) at least 99%, at least 99.5%, or 100% identical to SEQ ID NO: 195 (and, e.g., retaining the activity of native FIX). Optionally, the FIX coding sequence in the above examples encodes a FIX protein comprising the sequence set forth in SEQ ID NO: 195. Optionally, the FIX coding sequence in the above examples encodes a FIX protein consisting essentially of the sequence set forth in SEQ ID NO: 195. Optionally, the FIX coding sequence in the above examples encodes a FIX protein consisting of the sequence set forth in SEQ ID NO: 195.

(4) Vectors

The F9 nucleic acid constructs disclosed herein can be provided in a vector for expression or for integration into and expression from a target genomic locus. A vector can comprise additional sequences such as, for example, replication origins, promoters, and genes encoding antibiotic resistance. A vector can also comprise nuclease agent components as disclosed elsewhere herein. For example, a vector can comprise a F9 nucleic acid construct, a CRISPR/Cas system (nucleic acids encoding Cas protein and gRNA), one or more components of a CRISPR/Cas system, or a combination thereof (e.g., a F9 nucleic acid construct and a gRNA). In some cases, a vector comprising a F9 nucleic acid construct does not comprise any components of the nuclease agents described herein (e.g., does not comprise a nucleic acid encoding a Cas protein and does not comprise a nucleic acid encoding a gRNA). Some such vectors comprise homology arms corresponding to target sites in the target genomic locus. Other such vectors do not comprise any homology arms.

Some vectors may be circular. Alternatively, the vector may be linear. The vector can be packaged for delivered via a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

The vectors can be, for example, viral vectors such as adeno-associated virus (AAV) vectors. The AAV may be any suitable serotype and may be a single-stranded AAV (ssAAV) or a self-complementary AAV (scAAV). Other exemplary viruses/viral vectors include retroviruses, lentiviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression, long-lasting expression (e.g., at least 1 week, 2 weeks, 1 month, 2 months, or 3 months), or permanent expression. Viral vector may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper virus to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include 10¹², 10¹³, 10¹⁴, 10¹⁵, and 10¹⁶ vector genomes/mL. Exemplary viral titers (e.g., AAV titers) include about 10¹², about 10¹³, about 10¹⁴, about 10¹⁵, and about 10¹⁶ vector genomes (vg)/mL, or between about 10¹² to about 10¹⁶, between about 10¹² to about 10¹⁵, between about 10¹² to about 10¹⁴, between about 10¹² to about 10¹³, between about 10¹³ to about 10¹⁶, between about 10¹⁴ to about 10¹⁶, between about 10¹⁵ to about 10¹⁶, or between about 10¹³ to about 10¹⁵ vg/mL. Other exemplary viral titers (e.g., AAV titers) include about 10¹², about 10¹³, about 10¹⁴, about 10¹⁵, and about 10¹⁶ vector genomes (vg)/kg of body weight, or between about 10¹² to about 10¹⁶, between about 10¹² to about 10¹⁵, between about 10¹² to about 10¹⁴, between about 10¹² to about 10¹³, between about 10¹³ to about 10¹⁶, between about 10¹⁴ to about 10¹⁶, between about 10¹⁵ to about 10¹⁶, or between about 10¹³ to about 10¹⁵ vg/kg of body weight. In one example, the viral titer is between about 10¹³ to about 10¹⁴ vg/mL or vg/kg. In another example, the viral titer is between about 10¹² to about 10¹³ vg/mL or vg/kg (e.g., between about 10¹² to about 10¹³ vg/kg). In another example, the viral titer is between about 10¹² to about 10¹⁴ vg/mL or vg/kg (e.g., between about 10¹² to about 10¹⁴ vg/kg). For example, the viral titer can be between about 1.5E12 to about 1.5E13 vg/kg, can be about 1.5E12 vg/kg, or can be about 1.5E13 vg/kg. In another example, the viral titer is about 2E13 vg/mL or vg/kg.

Adeno-associated viruses (AAVs) are endemic in multiple species including human and non-human primates (NHPs). At least 12 natural serotypes and hundreds of natural variants have been isolated and characterized to date. See, e.g., Li et al. (2020) Nat. Rev. Genet. 21:255-272, herein incorporated by reference in its entirety for all purposes. AAV particles are naturally composed of a non-enveloped icosahedral protein capsid containing a single-stranded DNA (ssDNA) genome. The DNA genome is flanked by two inverted terminal repeats (ITRs) which serve as the viral origins of replication and packaging signals. The rep gene encodes four proteins required for viral replication and packaging whilst the cap gene encodes the three structural capsid subunits which dictate the AAV serotype, and the Assembly Activating Protein (AAP) which promotes virion assembly in some serotypes.

Recombinant AAV (rAAV) is currently one of the most commonly used viral vectors used in gene therapy to treat human diseases by delivering therapeutic transgenes to target cells in vivo. Indeed, rAAV vectors are composed of icosahedral capsids similar to natural AAVs, but rAAV virions do not encapsidate AAV protein-coding or AAV replicating sequences. These viral vectors are non-replicating. The only viral sequences required in rAAV vectors are the two ITRs, which are needed to guide genome replication and packaging during manufacturing of the rAAV vector. rAAV genomes are devoid of AAV rep and cap genes, rendering them non-replicating in vivo. rAAV vectors are produced by expressing rep and cap genes along with additional viral helper proteins in trans, in combination with the intended transgene cassette flanked by AAV ITRs.

In therapeutic rAAV genomes, a gene expression cassette is placed between ITR sequences. Typically, rAAV genome cassettes comprise of a promoter to drive expression of a therapeutic transgene, followed by polyadenylation sequence. The ITRs flanking a rAAV expression cassette are usually derived from AAV2, the first serotype to be isolated and converted into a recombinant viral vector. Since then, most rAAV production methods rely on AAV2 Rep-based packaging systems. See, e.g., Colella et al. (2017) Mol. Ther. Methods Clin. Dev. 8:87-104, herein incorporated by reference in its entirety for all purposes.

Some non-limiting examples of ITRs that can be used include ITRs comprising, consisting essentially of, or consisting of SEQ ID NO: 196, SEQ ID NO: 197, or SEQ ID NO: 198. Other examples of ITRs comprise one or more mutations compared to SEQ ID NO: 196, SEQ ID NO: 197, or SEQ ID NO: 198 and can be at least 90%, at least 91%, at least 92%, at least 93%, at least 94%, at least 95%, at least 96%, at least 97%, at least 98%, or at least 99% identical to SEQ ID NO: 196, SEQ ID NO: 197, or SEQ ID NO: 198. In some rAAV genomes disclosed herein, the F9 nucleic acid construct is flanked on both sides by the same ITR (i.e., the ITR on the 5′ end, and the reverse complement of the ITR on the 3′ end). In one example, the ITR on each end can comprise, consist essentially of, or consist of SEQ ID NO: 196. In another example, the ITR on each end can comprise, consist essentially of, or consist of SEQ ID NO: 197. In one example, the ITR on at least one end comprises, consists essentially of, or consists of SEQ ID NO: 198. In one example, the ITR on the 5′ end comprises, consists essentially of, or consists of SEQ ID NO: 198. In one example, the ITR on the 3′ end comprises, consists essentially of, or consists of SEQ ID NO: 198. In one example, the ITR on each end can comprise, consist essentially of, or consist of SEQ ID NO: 198. In one example, the ITR on at least one end comprises, consists essentially of, or consists of SEQ ID NO: 196. In one example, the ITR on the 5′ end comprises, consists essentially of, or consists of SEQ ID NO: 196. In one example, the ITR on the 3′ end comprises, consists essentially of, or consists of SEQ ID NO: 196. In one example, the ITR on each end can comprise, consist essentially of, or consist of SEQ ID NO: 196. In other rAAV genomes disclosed herein, the F9 nucleic acid construct is flanked by different ITRs on each end. In one example, the ITR on one end comprises, consists essentially of, or consists of SEQ ID NO: 196, and the ITR on the other end comprises, consists essentially of, or consists of SEQ ID NO: 197. In another example, the ITR on one end comprises, consists essentially of, or consists of SEQ ID NO: 196, and the ITR on the other end comprises, consists essentially of, or consists of SEQ ID NO: 198. In one example, the ITR on one end comprises, consists essentially of, or consists of SEQ ID NO: 197, and the ITR on the other end comprises, consists essentially of, or consists of SEQ ID NO: 198.

The specific serotype of a recombinant AAV vector influences its in-vivo tropism to specific tissues. AAV capsid proteins are responsible for mediating attachment and entry into target cells, followed by endosomal escape and trafficking to the nucleus. Thus, the choice of serotype when developing a rAAV vector will influence what cell types and tissues the vector is most likely to bind to and transduce when injected in vivo. Several serotypes of rAAVs, including rAAV8, are capable of transducing the liver when delivered systemically in mice, NHPs and humans. See, e.g., Li et al. (2020) Nat. Rev. Genet. 21:255-272, herein incorporated by reference in its entirety for all purposes.

Once in the nucleus, the ssDNA genome is released from the virion and a complementary DNA strand is synthesized to generate a double-stranded DNA (dsDNA) molecule. Double-stranded AAV genomes naturally circularize via their ITRs and become episomes which will persist extrachromosomally in the nucleus. Therefore, for episomal gene therapy programs, rAAV-delivered rAAV episomes provide long-term, promoter-driven gene expression in non-dividing cells. However, this rAAV-delivered episomal DNA is diluted out as cells divide. In contrast, the gene therapy described herein is based on gene insertion to allow long-term gene expression.

When specific rAAVs comprising specific sequences (e.g., specific bidirectional construct sequences or specific unidirectional construct sequences) are disclosed herein, they are meant to encompass the sequence disclosed or the reverse complement of the sequence. For example, if a bidirectional or unidirectional construct disclosed herein consists of the hypothetical sequence 5′-CTGGACCGA-3′, it is also meant to encompass the reverse complement of that sequence (5′-TCGGTCCAG-3′). Likewise, when rAAVs comprising bidirectional or unidirectional construct elements in a specific 5′ to 3′ order are disclosed herein, they are also meant to encompass the reverse complement of the order of those elements. For example, if an rAAV is disclosed herein that comprises a bidirectional construct that comprises from 5′ to 3′ a first splice acceptor, a first coding sequence, a first terminator, a reverse complement of a second terminator, a reverse complement of a second coding sequence, and a reverse complement of a second splice acceptor, it is also meant to encompass a construct comprising from 5′ to 3′ the second splice acceptor, the second coding sequence, the second terminator, a reverse complement of the first terminator, a reverse complement of the first coding sequence, and a reverse complement of the first splice acceptor. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediate AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. The term AAV includes, for example, AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. An “AAV vector” as used herein refers to an AAV vector comprising a heterologous sequence not of AAV origin (i.e., a nucleic acid sequence heterologous to AAV), typically comprising a sequence encoding a heterologous polypeptide of interest. The construct may comprise an AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV capsid sequence. In general, the heterologous nucleic acid sequence (the transgene) is flanked by at least one, and generally by two, AAV inverted terminal repeat sequences (ITRs). An AAV vector may either be single-stranded (ssAAV) or self-complementary (scAAV). Examples of serotypes for liver tissue include AAV3B, AAV5, AAV6, AAV7, AAV8, AAV9, AAVrh.74, and AAVhu.37, and particularly AAV8. In a specific example, the AAV vector comprising the nucleic acid construct can be recombinant AAV8 (rAAV8). A rAAV8 vector as described herein is one in which the capsid is from AAV8. For example, an AAV vector using ITRs from AAV2 and a capsid of AAV8 is considered herein to be a rAAV8 vector.

Tropism can be further refined through pseudotyping, which is the mixing of a capsid and a genome from different viral serotypes. For example AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell's DNA replication machinery to synthesize the complementary strand of the AAV's single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

The vector (e.g., AAV such as recombinant AAV8) can be formulated, for example, in 10 mM sodium phosphate, 180 mM sodium chloride, and 0.005% poloxamer 188, at pH 7.3.

B. Nuclease Agents and CRISPR/Cas Systems

The methods and compositions disclosed herein can utilize nuclease agents such as Clustered Regularly Interspersed Short Palindromic Repeats (CRISPR)/CRISPR-associated (Cas) systems, zinc finger nuclease (ZFN) systems, or Transcription Activator-Like Effector Nuclease (TALEN) systems or components of such systems to modify a target genomic locus in a target gene such as a safe harbor gene (e.g., ALB) for insertion of a F9 nucleic acid construct as disclosed herein. Generally, the nuclease agents involve the use of engineered cleavage systems to induce a double strand break or a nick (i.e., a single strand break) in a nuclease target site. Cleavage or nicking can occur through the use of specific nucleases such as engineered ZFNs, TALENs, or CRISPR/Cas systems with an engineered guide RNA to guide specific cleavage or nicking of the nuclease target site. Any nuclease agent that induces a nick or double-strand break at a desired target sequence can be used in the methods and compositions disclosed herein. The nuclease agent can be used to create a site of insertion at a desired locus (target gene) within a host genome, at which site the F9 nucleic acid construct is inserted to express FIX. The FIX may be heterologous with respect to its insertion site or locus (target gene), such as a safe harbor locus from which FIX is not normally expressed. Alternatively, the FIX may be non-heterologous with respect to its insertion site, such as insertion into the endogenous F9 locus to correct a defective F9 gene.

In one example, the nuclease agent is a CRISPR/Cas system. In another example, the nuclease agent comprises one or more ZFNs. In yet another example, the nuclease agent comprises one or more TALENs. In a specific example, the CRISPR/Cas systems or components of such systems target an ALB gene or locus (e.g., ALB genomic locus) within a cell, or intron 1 of an ALB gene or locus within a cell. In a more specific example, the CRISPR/Cas systems or components of such systems target a human ALB gene or locus or intron 1 of a human ALB gene or locus within a cell.

CRISPR/Cas systems include transcripts and other elements involved in the expression of, or directing the activity of, Cas genes. A CRISPR/Cas system can be, for example, a type I, a type II, a type III system, or a type V system (e.g., subtype V-A or subtype V-B). The methods and compositions disclosed herein can employ CRISPR/Cas systems by utilizing CRISPR complexes (comprising a guide RNA (gRNA) complexed with a Cas protein) for site-directed binding or cleavage of nucleic acids. A CRISPR/Cas system targeting an ALB gene or locus comprises a Cas protein (or a nucleic acid encoding the Cas protein) and one or more guide RNAs (or DNAs encoding the one or more guide RNAs), with each of the one or more guide RNAs targeting a different guide RNA target sequence in the target genomic locus (e.g., ALB gene or locus).

CRISPR/Cas systems used in the compositions and methods disclosed herein can be non-naturally occurring. A non-naturally occurring system includes anything indicating the involvement of the hand of man, such as one or more components of the system being altered or mutated from their naturally occurring state, being at least substantially free from at least one other component with which they are naturally associated in nature, or being associated with at least one other component with which they are not naturally associated. For example, some CRISPR/Cas systems employ non-naturally occurring CRISPR complexes comprising a gRNA and a Cas protein that do not naturally occur together, employ a Cas protein that does not occur naturally, or employ a gRNA that does not occur naturally.

(1) Target Genomic Loci and Albumin (ALB)

Any target genomic locus capable of expressing a gene can be used, such as a safe harbor locus (safe harbor gene, such as ALB) or an endogenous F9 locus. The nucleic acid construct can be integrated into any part of the target genomic locus. For example, the nucleic acid construct can be inserted into an intron or an exon of a target genomic locus or can replace one or more introns and/or exons of a target genomic locus. In a specific example, the nucleic acid construct can be integrated into an intron of the target genomic locus, such as the first intron of the target genomic locus (e.g., ALB intron 1). See, e.g., WO 2020/082042, US 2020/0270617, WO 2020/082041, US 2020/0268906, WO 2020/082046, and US 2020/0289628, each of which is herein incorporated by reference in its entirety for all purposes. Constructs integrated into a target genomic locus can be operably linked to an endogenous promoter at the target genomic locus (e.g., the endogenous ALB promoter).

Interactions between integrated exogenous DNA and a host genome can limit the reliability and safety of integration and can lead to overt phenotypic effects that are not due to the targeted genetic modification but are instead due to unintended effects of the integration on surrounding endogenous genes. For example, randomly inserted transgenes can be subject to position effects and silencing, making their expression unreliable and unpredictable. Likewise, integration of exogenous DNA into a chromosomal locus can affect surrounding endogenous genes and chromatin, thereby altering cell behavior and phenotypes. Safe harbor loci include chromosomal loci where transgenes or other exogenous nucleic acid inserts can be stably and reliably expressed in all tissues of interest without overtly altering cell behavior or phenotype (i.e., without any deleterious effects on the host cell). See, e.g., Sadelain et al. (2012) Nat. Rev. Cancer 12:51-58, herein incorporated by reference in its entirety for all purposes. For example, the safe harbor locus can be one in which expression of the inserted gene sequence is not perturbed by any read-through expression from neighboring genes. For example, safe harbor loci can include chromosomal loci where exogenous DNA can integrate and function in a predictable manner without adversely affecting endogenous gene structure or expression. Safe harbor loci can include extragenic regions or intragenic regions such as, for example, loci within genes that are non-essential, dispensable, or able to be disrupted without overt phenotypic consequences.

Such safe harbor loci can offer an open chromatin configuration in all tissues and can be ubiquitously expressed during embryonic development and in adults. See, e.g., Zambrowicz et al. (1997) Proc. Natl. Acad. Sci. U.S.A. 94:3789-3794, herein incorporated by reference in its entirety for all purposes. In addition, the safe harbor loci can be targeted with high efficiency, and safe harbor loci can be disrupted with no overt phenotype. Examples of safe harbor loci include ALB, CCR5, HPRT, AAVS1, and Rosa26. See, e.g., U.S. Pat. Nos. 7,888,121; 7,972,854; 7,914,796; 7,951,925; 8,110,379; 8,409,861; 8,586,526; and US Patent Publication Nos. 2003/0232410; 2005/0208489; 2005/0026157; 2006/0063231; 2008/0159996; 2010/00218264; 2012/0017290; 2011/0265198; 2013/0137104; 2013/0122591; 2013/0177983; 2013/0177960; and 2013/0122591, each of which is herein incorporated by reference in its entirety for all purposes.

In a specific example, a safe harbor locus is a locus within the genome wherein a gene may be inserted without significant deleterious effects on the host cell such as a hepatocyte (e.g., without causing apoptosis, necrosis, and/or senescence, or without causing more than 5%, 10%, 15%, 20%, 25%, 30%, or 40% apoptosis, necrosis, and/or senescence as compared to a control cell). The safe harbor locus can allow overexpression of an exogenous gene without significant deleterious effects on the host cell such as a hepatocyte (e.g., without causing apoptosis, necrosis, and/or senescence, or without causing more than 5%, 10%, 15%, 20%, 25%, 30%, or 40% apoptosis, necrosis, and/or senescence as compared to a control cell). A desirable safe harbor locus may be one in which expression of the inserted gene sequence is not perturbed by read-through expression from neighboring genes. The safe harbor may be a human safe harbor (e.g., for a liver tissue or hepatocyte host cell).

In a specific example, the target genomic locus is an ALB locus, such as intron 1 of an ALB locus. In a more specific example, the target genomic locus is a human ALB locus, such as intron 1 of a human ALB locus (e.g., SEQ ID NO: 4).

(2) Cas Proteins

Cas proteins generally comprise at least one RNA recognition or binding domain that can interact with guide RNAs. Cas proteins can also comprise nuclease domains (e.g., DNase domains or RNase domains), DNA-binding domains, helicase domains, protein-protein interaction domains, dimerization domains, and other domains. Some such domains (e.g., DNase domains) can be from a native Cas protein. Other such domains can be added to make a modified Cas protein. A nuclease domain possesses catalytic activity for nucleic acid cleavage, which includes the breakage of the covalent bonds of a nucleic acid molecule. Cleavage can produce blunt ends or staggered ends, and it can be single-stranded or double-stranded. For example, a wild type Cas9 protein will typically create a blunt cleavage product. Alternatively, a wild type Cpf1 protein (e.g., FnCpf1) can result in a cleavage product with a 5-nucleotide 5′ overhang, with the cleavage occurring after the 18th base pair from the PAM sequence on the non-targeted strand and after the 23rd base on the targeted strand. A Cas protein can have full cleavage activity to create a double-strand break at a target genomic locus (e.g., a double-strand break with blunt ends), or it can be a nickase that creates a single-strand break at a target genomic locus.

Examples of Cas proteins include Cas1, Cas1B, Cas2, Cas3, Cas4, Cas5, Cas5e (CasD), Cash, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

An exemplary Cas protein is a Cas9 protein or a protein derived from a Cas9 protein. Cas9 proteins are from a type II CRISPR/Cas system and typically share four key motifs with a conserved architecture. Motifs 1, 2, and 4 are RuvC-like motifs, and motif 3 is an HNH motif. Exemplary Cas9 proteins are from Streptococcus pyogenes, Streptococcus thermophilus, Streptococcus sp., Staphylococcus aureus, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Neisseria meningitidis, or Campylobacter jejuni. Additional examples of the Cas9 family members are described in WO 2014/131833, herein incorporated by reference in its entirety for all purposes. Cas9 from S. pyogenes (SpCas9) (e.g., assigned UniProt accession number Q99ZW2) is an exemplary Cas9 protein. An exemplary SpCas9 protein sequence is set forth in SEQ ID NO: 8 (encoded by the DNA sequence set forth in SEQ ID NO: 9). An exemplary SpCas9 mRNA (cDNA) sequence is set forth in SEQ ID NO: 10. Smaller Cas9 proteins (e.g., Cas9 proteins whose coding sequences are compatible with the maximum AAV packaging capacity when combined with a guide RNA coding sequence and regulatory elements for the Cas9 and guide RNA, such as SaCas9 and CjCas9 and Nme2Cas9) are other exemplary Cas9 proteins. For example, Cas9 from S. aureus (SaCas9) (e.g., assigned UniProt accession number J7RUA5) is another exemplary Cas9 protein. Likewise, Cas9 from Campylobacter jejuni (CjCas9) (e.g., assigned UniProt accession number QOP897) is another exemplary Cas9 protein. See, e.g., Kim et al. (2017) Nat. Commun. 8:14500, herein incorporated by reference in its entirety for all purposes. SaCas9 is smaller than SpCas9, and CjCas9 is smaller than both SaCas9 and SpCas9. Cas9 from Neisseria meningitidis (Nme2Cas9) is another exemplary Cas9 protein. See, e.g., Edraki et al. (2019) Mol. Cell 73(4):714-726, herein incorporated by reference in its entirety for all purposes. Cas9 proteins from Streptococcus thermophilus (e.g., Streptococcus thermophilus LMD-9 Cas9 encoded by the CRISPR1 locus (St1Cas9) or Streptococcus thermophilus Cas9 from the CRISPR3 locus (St3Cas9)) are other exemplary Cas9 proteins. Cas9 from Francisella novicida (FnCas9) or the RHA Francisella novicida Cas9 variant that recognizes an alternative PAM (E1369R/E1449H/R1556A substitutions) are other exemplary Cas9 proteins. These and other exemplary Cas9 proteins are reviewed, e.g., in Cebrian-Serrano and Davies (2017) Mamm. Genome 28(7):247-261, herein incorporated by reference in its entirety for all purposes. Examples of Cas9 coding sequences, Cas9 mRNAs, and Cas9 protein sequences are provided in WO 2013/176772, WO 2014/065596, WO 2016/106121, WO 2019/067910, WO 2020/082042, US 2020/0270617, WO 2020/082041, US 2020/0268906, WO 2020/082046, and US 2020/0289628, each of which is herein incorporated by reference in its entirety for all purposes. Specific examples of ORFs and Cas9 amino acid sequences are provided in Table 30 at paragraph [0449] WO 2019/067910, and specific examples of Cas9 mRNAs and ORFs are provided in paragraphs [0214]-[0234] of WO 2019/067910. See also WO 2020/082046 A2 (pp. 84-85) and Table 24 in WO 2020/069296, each of which is herein incorporated by reference in its entirety for all purposes. An exemplary SpCas9 protein sequence comprises, consists essentially of, or consists of SEQ ID NO: 11. An exemplary SpCas9 mRNA sequence encoding that SpCas9 protein sequence comprises, consists essentially of, or consists of SEQ ID NO: 12. Another exemplary SpCas9 mRNA sequence encoding that SpCas9 protein sequence comprises, consists essentially of, or consists of SEQ ID NO: 225. Another exemplary SpCas9 mRNA sequence encoding that SpCas9 protein sequence comprises SEQ ID NO: 226. An exemplary SpCas9 coding sequence comprises, consists essentially of, or consists of SEQ ID NO: 227.

Another example of a Cas protein is a Cpf1 (CRISPR from Prevotella and Francisella 1) protein. Cpf1 is a large protein (about 1300 amino acids) that contains a RuvC-like nuclease domain homologous to the corresponding domain of Cas9 along with a counterpart to the characteristic arginine-rich cluster of Cas9. However, Cpf1 lacks the HNH nuclease domain that is present in Cas9 proteins, and the RuvC-like domain is contiguous in the Cpf1 sequence, in contrast to Cas9 where it contains long inserts including the HNH domain. See, e.g., Zetsche et al. (2015) Cell 163(3):759-771, herein incorporated by reference in its entirety for all purposes. Exemplary Cpf1 proteins are from Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, and Porphyromonas macacae. Cpf1 from Francisella novicida U112 (FnCpf1; assigned UniProt accession number A0Q7Q2) is an exemplary Cpf1 protein.

Another example of a Cas protein is CasX (Cas12e). CasX is an RNA-guided DNA endonuclease that generates a staggered double-strand break in DNA. CasX is less than 1000 amino acids in size. Exemplary CasX proteins are from Deltaproteobacteria (DpbCasX or DpbCas12e) and Planctomycetes (P1mCasX or PlmCas12e). Like Cpf1, CasX uses a single RuvC active site for DNA cleavage. See, e.g., Liu et al. (2019) Nature 566(7743):218-223, herein incorporated by reference in its entirety for all purposes.

Another example of a Cas protein is CasΦ (CasPhi or Cas12j), which is uniquely found in bacteriophages. CasΦ is less than 1000 amino acids in size (e.g., 700-800 amino acids). CasΦ cleavage generates staggered 5′ overhangs. A single RuvC active site in CasΦ is capable of crRNA processing and DNA cutting. See, e.g., Pausch et al. (2020) Science 369(6501):333-337, herein incorporated by reference in its entirety for all purposes.

Cas proteins can be wild type proteins (i.e., those that occur in nature), modified Cas proteins (i.e., Cas protein variants), or fragments of wild type or modified Cas proteins. Cas proteins can also be active variants or fragments with respect to catalytic activity of wild type or modified Cas proteins. Active variants or fragments with respect to catalytic activity can comprise at least 80%, 85%, 90%, 91%, 92%, 93%, 94%, 95%, 96%, 97%, 98%, 99% or more sequence identity to the wild type or modified Cas protein or a portion thereof, wherein the active variants retain the ability to cut at a desired cleavage site and hence retain nick-inducing or double-strand-break-inducing activity. Assays for nick-inducing or double-strand-break-inducing activity are known and generally measure the overall activity and specificity of the Cas protein on DNA substrates containing the cleavage site.

One example of a modified Cas protein is the modified SpCas9-HF1 protein, which is a high-fidelity variant of Streptococcus pyogenes Cas9 harboring alterations (N497A/R661A/Q695A/Q926A) designed to reduce non-specific DNA contacts. See, e.g., Kleinstiver et al. (2016) Nature 529(7587):490-495, herein incorporated by reference in its entirety for all purposes. Another example of a modified Cas protein is the modified eSpCas9 variant (K848A/K1003A/R1060A) designed to reduce off-target effects. See, e.g., Slaymaker et al. (2016) Science 351(6268):84-88, herein incorporated by reference in its entirety for all purposes. Other SpCas9 variants include K855A and K810A/K1003A/R1060A. These and other modified Cas proteins are reviewed, e.g., in Cebrian-Serrano and Davies (2017) Mamm. Genome 28(7):247-261, herein incorporated by reference in its entirety for all purposes. Another example of a modified Cas9 protein is xCas9, which is a SpCas9 variant that can recognize an expanded range of PAM sequences. See, e.g., Hu et al. (2018) Nature 556:57-63, herein incorporated by reference in its entirety for all purposes.

Cas proteins can be modified to increase or decrease one or more of nucleic acid binding affinity, nucleic acid binding specificity, and enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of or a property of the Cas protein.

Cas proteins can comprise at least one nuclease domain, such as a DNase domain. For example, a wild type Cpf1 protein generally comprises a RuvC-like domain that cleaves both strands of target DNA, perhaps in a dimeric configuration. Likewise, CasX and CasΦ generally comprise a single RuvC-like domain that cleaves both strands of a target DNA. Cas proteins can also comprise at least two nuclease domains, such as DNase domains. For example, a wild type Cas9 protein generally comprises a RuvC-like nuclease domain and an HNH-like nuclease domain. The RuvC and HNH domains can each cut a different strand of double-stranded DNA to make a double-stranded break in the DNA. See, e.g., Jinek et al. (2012) Science 337(6096):816-821, herein incorporated by reference in its entirety for all purposes.

One or more of the nuclease domains can be deleted or mutated so that they are no longer functional or have reduced nuclease activity. For example, if one of the nuclease domains is deleted or mutated in a Cas9 protein, the resulting Cas9 protein can be referred to as a nickase and can generate a single-strand break within a double-stranded target DNA but not a double-strand break (i.e., it can cleave the complementary strand or the non-complementary strand, but not both). If none of the nuclease domains is deleted or mutated in a Cas9 protein, the Cas9 protein will retain double-strand-break-inducing activity. An example of a mutation that converts Cas9 into a nickase is a D10A (aspartate to alanine at position 10 of Cas9) mutation in the RuvC domain of Cas9 from S. pyogenes. Likewise, H939A (histidine to alanine at amino acid position 839), H840A (histidine to alanine at amino acid position 840), or N863A (asparagine to alanine at amino acid position N863) in the HNH domain of Cas9 from S. pyogenes can convert the Cas9 into a nickase. Other examples of mutations that convert Cas9 into a nickase include the corresponding mutations to Cas9 from S. thermophilus. See, e.g., Sapranauskas et al. (2011) Nucleic Acids Res. 39(21):9275-9282 and WO 2013/141680, each of which is herein incorporated by reference in its entirety for all purposes. Such mutations can be generated using methods such as site-directed mutagenesis, PCR-mediated mutagenesis, or total gene synthesis. Examples of other mutations creating nickases can be found, for example, in WO 2013/176772 and WO 2013/142578, each of which is herein incorporated by reference in its entirety for all purposes.

Examples of inactivating mutations in the catalytic domains of xCas9 are the same as those described above for SpCas9. Examples of inactivating mutations in the catalytic domains of Staphylococcus aureus Cas9 proteins are also known. For example, the Staphylococcus aureus Cas9 enzyme (SaCas9) may comprise a substitution at position N580 (e.g., N580A substitution) or a substitution at position D10 (e.g., D10A substitution) to generate a Cas nickase. See, e.g., WO 2016/106236, herein incorporated by reference in its entirety for all purposes. Examples of inactivating mutations in the catalytic domains of Nme2Cas9 are also known (e.g., D16A or H588A). Examples of inactivating mutations in the catalytic domains of St1Cas9 are also known (e.g., D9A, D598A, H599A, or N622A). Examples of inactivating mutations in the catalytic domains of St3Cas9 are also known (e.g., D10A or N870A). Examples of inactivating mutations in the catalytic domains of CjCas9 are also known (e.g., combination of D8A or H559A). Examples of inactivating mutations in the catalytic domains of FnCas9 and RHA FnCas9 are also known (e.g., N995A).

Examples of inactivating mutations in the catalytic domains of Cpf1 proteins are also known. With reference to Cpf1 proteins from Francisella novicida U112 (FnCpf1), Acidaminococcus sp. BV3L6 (AsCpf1), Lachnospiraceae bacterium ND2006 (LbCpf1), and Moraxella bovoculi 237 (MbCpf1 Cpf1), such mutations can include mutations at positions 908, 993, or 1263 of AsCpf1 or corresponding positions in Cpf1 orthologs, or positions 832, 925, 947, or 1180 of LbCpf1 or corresponding positions in Cpf1 orthologs. Such mutations can include, for example one or more of mutations D908A, E993A, and D1263A of AsCpf1 or corresponding mutations in Cpf1 orthologs, or D832A, E925A, D947A, and D1180A of LbCpf1 or corresponding mutations in Cpf1 orthologs. See, e.g., US 2016/0208243, herein incorporated by reference in its entirety for all purposes.

Examples of inactivating mutations in the catalytic domains of CasX proteins are also known. With reference to CasX proteins from Deltaproteobacteria, D672A, E769A, and D935A (individually or in combination) or corresponding positions in other CasX orthologs are inactivating. See, e.g., Liu et al. (2019) Nature 566(7743):218-223, herein incorporated by reference in its entirety for all purposes.

Examples of inactivating mutations in the catalytic domains of CasΦ proteins are also known. For example, D371A and D394A, alone or in combination, are inactivating mutations. See, e.g., Pausch et al. (2020) Science 369(6501):333-337, herein incorporated by reference in its entirety for all purposes.

Cas proteins can also be operably linked to heterologous polypeptides as fusion proteins. For example, a Cas protein can be fused to a cleavage domain. See WO 2014/089290, herein incorporated by reference in its entirety for all purposesCas proteins can also be fused to a heterologous polypeptide providing increased or decreased stability. The fused domain or heterologous polypeptide can be located at the N-terminus, the C-terminus, or internally within the Cas protein.

As one example, a Cas protein can be fused to one or more heterologous polypeptides that provide for subcellular localization. Such heterologous polypeptides can include, for example, one or more nuclear localization signals (NLS) such as the monopartite SV40 NLS and/or a bipartite alpha-importin NLS for targeting to the nucleus, a mitochondrial localization signal for targeting to the mitochondria, an ER retention signal, and the like. See, e.g., Lange et al. (2007) J. Biol. Chem. 282(8):5101-5105, herein incorporated by reference in its entirety for all purposes. Such subcellular localization signals can be located at the N-terminus, the C-terminus, or anywhere within the Cas protein. An NLS can comprise a stretch of basic amino acids, and can be a monopartite sequence or a bipartite sequence. Optionally, a Cas protein can comprise two or more NLSs, including an NLS (e.g., an alpha-importin NLS or a monopartite NLS) at the N-terminus and an NLS (e.g., an SV40 NLS or a bipartite NLS) at the C-terminus. A Cas protein can also comprise two or more NLSs at the N-terminus and/or two or more NLSs at the C-terminus.

A Cas protein may, for example, be fused with 1-10 NLSs (e.g., fused with 1-5 NLSs or fused with one NLS. Where one NLS is used, the NLS may be linked at the N-terminus or the C-terminus of the Cas protein sequence. It may also be inserted within the Cas protein sequence. Alternatively, the Cas protein may be fused with more than one NLS. For example, the Cas protein may be fused with 2, 3, 4, or 5 NLSs. In a specific example, the Cas protein may be fused with two NLSs. In certain circumstances, the two NLSs may be the same (e.g., two SV40 NLSs) or different. For example, the Cas protein can be fused to two SV40 NLS sequences linked at the carboxy terminus. Alternatively, the Cas protein may be fused with two NLSs, one linked at the N-terminus and one at the C-terminus. In other examples, the Cas protein may be fused with 3 NLSs or with no NLS. The NLS may be a monopartite sequence, such as, e.g., the SV40 NLS, PKKKRKV (SEQ ID NO: 13) or PKKKRRV (SEQ ID NO: 14). The NLS may be a bipartite sequence, such as the NLS of nucleoplasmin, KRPAATKKAGQAKKKK (SEQ ID NO: 15). In a specific example, a single PKKKRKV (SEQ ID NO: 13) NLS may be linked at the C-terminus of the Cas protein. One or more linkers are optionally included at the fusion site.

Cas proteins can also be operably linked to a cell-penetrating domain or protein transduction domain. For example, the cell-penetrating domain can be derived from the HIV-1 TAT protein, the TLM cell-penetrating motif from human hepatitis B virus, MPG, Pep-1, VP22, a cell penetrating peptide from Herpes simplex virus, or a polyarginine peptide sequence. See, e.g., WO 2014/089290 and WO 2013/176772, each of which is herein incorporated by reference in its entirety for all purposes. The cell-penetrating domain can be located at the N-terminus, the C-terminus, or anywhere within the Cas protein.

Cas proteins can also be operably linked to a heterologous polypeptide for ease of tracking or purification, such as a fluorescent protein, a purification tag, or an epitope tag. Examples of fluorescent proteins include green fluorescent proteins (e.g., GFP, GFP-2, tagGFP, turboGFP, eGFP, Emerald, Azami Green, Monomeric Azami Green, CopGFP, AceGFP, ZsGreen1), yellow fluorescent proteins (e.g., YFP, eYFP, Citrine, Venus, YPet, PhiYFP, ZsYellow1), blue fluorescent proteins (e.g., eBFP, eBFP2, Azurite, mKalama1, GFPuv, Sapphire, T-sapphire), cyan fluorescent proteins (e.g., eCFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRaspberry, mStrawberry, Jred), orange fluorescent proteins (e.g., mOrange, mKO, Kusabira-Orange, Monomeric Kusabira-Orange, mTangerine, tdTomato), and any other suitable fluorescent protein. Examples of tags include glutathione-S-transferase (GST), chitin binding protein (CBP), maltose binding protein, thioredoxin (TRX), poly(NANP), tandem affinity purification (TAP) tag, myc, AcV5, AU1, AUS, E, ECS, E2, FLAG, hemagglutinin (HA), nus, Softag 1, Softag 3, Strep, SBP, Glu-Glu, HSV, KT3, S, 51, T7, V5, VSV-G, histidine (His), biotin carboxyl carrier protein (BCCP), and calmodulin.

Cas proteins can also be tethered to labeled nucleic acids. Such tethering (i.e., physical linking) can be achieved through covalent interactions or noncovalent interactions, and the tethering can be direct (e.g., through direct fusion or chemical conjugation, which can be achieved by modification of cysteine or lysine residues on the protein or intein modification), or can be achieved through one or more intervening linkers or adapter molecules such as streptavidin or aptamers. See, e.g., Pierce et al. (2005) Mini Rev. Med. Chem. 5(1):41-55; Duckworth et al. (2007) Angew. Chem. Int. Ed. Engl. 46(46):8819-8822; Schaeffer and Dixon (2009) Australian J. Chem. 62(10):1328-1332; Goodman et al. (2009) Chembiochem. 10(9):1551-1557; and Khatwani et al. (2012) Bioorg. Med. Chem. 20(14):4532-4539, each of which is herein incorporated by reference in its entirety for all purposes. Noncovalent strategies for synthesizing protein-nucleic acid conjugates include biotin-streptavidin and nickel-histidine methods. Covalent protein-nucleic acid conjugates can be synthesized by connecting appropriately functionalized nucleic acids and proteins using a wide variety of chemistries. Some of these chemistries involve direct attachment of the oligonucleotide to an amino acid residue on the protein surface (e.g., a lysine amine or a cysteine thiol), while other more complex schemes require post-translational modification of the protein or the involvement of a catalytic or reactive protein domain. Methods for covalent attachment of proteins to nucleic acids can include, for example, chemical cross-linking of oligonucleotides to protein lysine or cysteine residues, expressed protein-ligation, chemoenzymatic methods, and the use of photoaptamers. The labeled nucleic acid can be tethered to the C-terminus, the N-terminus, or to an internal region within the Cas protein. In one example, the labeled nucleic acid is tethered to the C-terminus or the N-terminus of the Cas protein. Likewise, the Cas protein can be tethered to the 5′ end, the 3′ end, or to an internal region within the labeled nucleic acid. That is, the labeled nucleic acid can be tethered in any orientation and polarity. For example, the Cas protein can be tethered to the 5′ end or the 3′ end of the labeled nucleic acid.

Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Optionally, the nucleic acid encoding the Cas protein can be codon optimized for efficient translation into protein in a particular cell or organism. For example, the nucleic acid encoding the Cas protein can be modified to substitute codons having a higher frequency of usage in a bacterial cell, a yeast cell, a human cell, a non-human cell, a mammalian cell, a rodent cell, a mouse cell, a rat cell, or any other host cell of interest, as compared to the naturally occurring polynucleotide sequence. When a nucleic acid encoding the Cas protein is introduced into the cell, the Cas protein can be transiently, conditionally, or constitutively expressed in the cell.

Nucleic acids encoding Cas proteins can be stably integrated in the genome of a cell and operably linked to a promoter active in the cell. Alternatively, nucleic acids encoding Cas proteins can be operably linked to a promoter in an expression construct. Expression constructs include any nucleic acid constructs capable of directing expression of a gene or other nucleic acid sequence of interest (e.g., a Cas gene) and which can transfer such a nucleic acid sequence of interest to a target cell. For example, the nucleic acid encoding the Cas protein can be in a vector comprising a DNA encoding a gRNA. Alternatively, it can be in a vector or plasmid that is separate from the vector comprising the DNA encoding the gRNA. Promoters that can be used in an expression construct include promoters active, for example, in one or more of a eukaryotic cell, a human cell, a non-human cell, a mammalian cell, a non-human mammalian cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Optionally, the promoter can be a bidirectional promoter driving expression of both a Cas protein in one direction and a guide RNA in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express genes encoding a Cas protein and a guide RNA simultaneously allow for the generation of compact expression cassettes to facilitate delivery.

Different promoters can be used to drive Cas expression or Cas9 expression. In some methods, small promoters are used so that the Cas or Cas9 coding sequence can fit into an AAV construct. For example, Cas or Cas9 and one or more gRNAs (e.g., 1 gRNA or 2 gRNAs or 3 gRNAs or 4 gRNAs) can be delivered via LNP-mediated delivery (e.g., in the form of RNA) or adeno-associated virus (AAV)-mediated delivery (e.g., AAV2-mediated delivery, AAV5-mediated delivery, AAV8-mediated delivery, or AAV7m8-mediated delivery). For example, the nuclease agent can be CRISPR/Cas9, and a Cas9 mRNA and a gRNA targeting an intron 1 of an endogenous human ALB locus can be delivered via LNP-mediated delivery or AAV-mediated delivery. The Cas or Cas9 and the gRNA(s) can be delivered in a single AAV or via two separate AAVs. For example, a first AAV can carry a Cas or Cas9 expression cassette, and a second AAV can carry a gRNA expression cassette. Similarly, a first AAV can carry a Cas or Cas9 expression cassette, and a second AAV can carry two or more gRNA expression cassettes. Alternatively, a single AAV can carry a Cas or Cas9 expression cassette (e.g., Cas or Cas9 coding sequence operably linked to a promoter) and a gRNA expression cassette (e.g., gRNA coding sequence operably linked to a promoter). Similarly, a single AAV can carry a Cas or Cas9 expression cassette (e.g., Cas or Cas9 coding sequence operably linked to a promoter) and two or more gRNA expression cassettes (e.g., gRNA coding sequences operably linked to promoters). Different promoters can be used to drive expression of the gRNA, such as a U6 promoter or the small tRNA Gln. Likewise, different promoters can be used to drive Cas9 expression. For example, small promoters are used so that the Cas9 coding sequence can fit into an AAV construct. Similarly, small Cas9 proteins (e.g., SaCas9 or CjCas9 are used to maximize the AAV packaging capacity).

Cas proteins provided as mRNAs can be modified for improved stability and/or immunogenicity properties. The modifications may be made to one or more nucleosides within the mRNA. Examples of chemical modifications to mRNA nucleobases include pseudouridine, 1-methyl-pseudouridine, and 5-methyl-cytidine. mRNA encoding Cas proteins can also be capped. The cap can be, for example, a cap 1 structure in which the +1 ribonucleotide is methylated at the 2′O position of the ribose. The capping can, for example, give superior activity in vivo (e.g., by mimicking a natural cap), can result in a natural structure that reduce stimulation of the innate immune system of the host (e.g., can reduce activation of pattern recognition receptors in the innate immune system). mRNA encoding Cas proteins can also be polyadenylated (to comprise a poly(A) tail). mRNA encoding Cas proteins can also be modified to include pseudouridine (e.g., can be fully substituted with pseudouridine). As another example, capped and polyadenylated Cas mRNA containing N1-methyl-pseudouridine can be used. mRNA encoding Cas proteins can also be modified to include N1-methyl-pseudouridine (e.g., can be fully substituted with N1-methyl-pseudouridine). As another example, Cas mRNA fully substituted with pseudouridine can be used (i.e., all standard uracil residues are replaced with pseudouridine, a uridine isomer in which the uracil is attached with a carbon-carbon bond rather than nitrogen-carbon). As another example, Cas mRNA fully substituted with N1-methyl-pseudouridine can be used (i.e., all standard uracil residues are replaced with N1-methyl-pseudouridine). Likewise, Cas mRNAs can be modified by depletion of uridine using synonymous codons. For example, capped and polyadenylated Cas mRNA fully substituted with pseudouridine can be used. For example, capped and polyadenylated Cas mRNA fully substituted with N1-methyl-pseudouridine can be used.

Cas mRNAs can comprise a modified uridine at least at one, a plurality of, or all uridine positions. The modified uridine can be a uridine modified at the 5 position (e.g., with a halogen, methyl, or ethyl). The modified uridine can be a pseudouridine modified at the 1 position (e.g., with a halogen, methyl, or ethyl). The modified uridine can be, for example, pseudouridine, N1-methyl-pseudouridine, 5-methoxyuridine, 5-iodouridine, or a combination thereof. In some examples, the modified uridine is 5-methoxyuridine. In some examples, the modified uridine is 5-iodouridine. In some examples, the modified uridine is pseudouridine. In some examples, the modified uridine is N1-methyl-pseudouridine. In some examples, the modified uridine is a combination of pseudouridine and N1-methyl-pseudouridine. In some examples, the modified uridine is a combination of pseudouridine and 5-methoxyuridine. In some examples, the modified uridine is a combination of N1-methyl pseudouridine and 5-methoxyuridine. In some examples, the modified uridine is a combination of 5-iodouridine and N1-methyl-pseudouridine. In some examples, the modified uridine is a combination of pseudouridine and 5-iodouridine. In some examples, the modified uridine is a combination of 5-iodouridine and 5-methoxyuridine.

Cas mRNAs disclosed herein can also comprise a 5′ cap, such as a Cap0, Cap1, or Cap2. A 5′ cap is generally a 7-methylguanine ribonucleotide (which may be further modified, e.g., with respect to ARCA) linked through a 5′-triphosphate to the 5′ position of the first nucleotide of the 5′-to-3′ chain of the mRNA (i.e., the first cap-proximal nucleotide). In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc. Natl. Acad. Sci. U.S.A. 111(33):12025-30 and Abbas et al. (2017) Proc. Natl. Acad. Sci. U.S.A. 114(11):E2106-E2115, each of which is herein incorporated by reference in its entirety for all purposes. Most endogenous higher eukaryotic mRNAs, including mammalian mRNAs such as human mRNAs, comprise Cap1 or Cap2. Cap0 and other cap structures differing from Cap1 and Cap2 may be immunogenic in mammals, such as humans, due to recognition as non-self by components of the innate immune system such as IFIT-1 and IFIT-5, which can result in elevated cytokine levels including type I interferon. Components of the innate immune system such as IFIT-1 and IFIT-5 may also compete with eIF4E for binding of an mRNA with a cap other than Cap1 or Cap2, potentially inhibiting translation of the mRNA.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al. (2001) RNA 7:1486-1495, herein incorporated by reference in its entirety for all purposes.

CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively.

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo and Moss (1990) Proc. Natl. Acad. Sci. U.S.A. 87:4023-4027 and Mao and Shuman (1994) J. Biol. Chem. 269:24472-24479, each of which is herein incorporated by reference in its entirety for all purposes.

Cas mRNAs can further comprise a poly-adenylated (poly-A or poly(A) or poly-adenine) tail. The poly-A tail can, for example, comprise at least 20, at least 30, at least 40, at least 50, at least 60, at least 70, at least 80, at least 90, or at least 100 adenines, and optionally up to 300 adenines. For example, the poly-A tail can comprise 95, 96, 97, 98, 99, or 100 adenine nucleotides.

(3) Guide RNAs

A “guide RNA” or “gRNA” is an RNA molecule that binds to a Cas protein (e.g., Cas9 protein) and targets the Cas protein to a specific location within a target DNA. Guide RNAs can comprise two segments: a “DNA-targeting segment” (also called “guide sequence”) and a “protein-binding segment.” “Segment” includes a section or region of a molecule, such as a contiguous stretch of nucleotides in an RNA. Some gRNAs, such as those for Cas9, can comprise two separate RNA molecules: an “activator-RNA” (e.g., tracrRNA) and a “targeter-RNA” (e.g., CRISPR RNA or crRNA). Other gRNAs are a single RNA molecule (single RNA polynucleotide), which can also be called a “single-molecule gRNA,” a “single-guide RNA,” or an “sgRNA.” See, e.g., WO 2013/176772, WO 2014/065596, WO 2014/089290, WO 2014/093622, WO 2014/099750, WO 2013/142578, and WO 2014/131833, each of which is herein incorporated by reference in its entirety for all purposes. A guide RNA can refer to either a CRISPR RNA (crRNA) or the combination of a crRNA and a trans-activating CRISPR RNA (tracrRNA). The crRNA and tracrRNA can be associated as a single RNA molecule (single guide RNA or sgRNA) or in two separate RNA molecules (dual guide RNA or dgRNA). For Cas9, for example, a single-guide RNA can comprise a crRNA fused to a tracrRNA (e.g., via a linker). For Cpf1 and CasΦ, for example, only a crRNA is needed to achieve binding to a target sequence. The terms “guide RNA” and “gRNA” include both double-molecule (i.e., modular) gRNAs and single-molecule gRNAs. In some of the methods and compositions disclosed herein, a gRNA is a S. pyogenes Cas9 gRNA or an equivalent thereof. In some of the methods and compositions disclosed herein, a gRNA is a S. aureus Cas9 gRNA or an equivalent thereof.

An exemplary two-molecule gRNA comprises a crRNA-like (“CRISPR RNA” or “targeter-RNA” or “crRNA” or “crRNA repeat”) molecule and a corresponding tracrRNA-like (“trans-activating CRISPR RNA” or “activator-RNA” or “tracrRNA”) molecule. A crRNA comprises both the DNA-targeting segment (single-stranded) of the gRNA and a stretch of nucleotides that forms one half of the dsRNA duplex of the protein-binding segment of the gRNA. An example of a crRNA tail (e.g., for use with S. pyogenes Cas9), located downstream (3′) of the DNA-targeting segment, comprises, consists essentially of, or consists of GUUUUAGAGCUAUGCU (SEQ ID NO: 16) or GUUUUAGAGCUAUGCUGUUUUG (SEQ ID NO: 17). Any of the DNA-targeting segments disclosed herein can be joined to the 5′ end of SEQ ID NO: 16 or 17 to form a crRNA.

A corresponding tracrRNA (activator-RNA) comprises a stretch of nucleotides that forms the other half of the dsRNA duplex of the protein-binding segment of the gRNA. A stretch of nucleotides of a crRNA are complementary to and hybridize with a stretch of nucleotides of a tracrRNA to form the dsRNA duplex of the protein-binding domain of the gRNA. As such, each crRNA can be said to have a corresponding tracrRNA. Examples of tracrRNA sequences (e.g., for use with S. pyogenes Cas9) comprise, consist essentially of, or consist of any one of

(SEQ ID NO: 18)
AGCAUAGCAAGUUAAAAUAAGGCUAGUCCGUUAUC
AACUUGAAAAAGUGGCACCGAGUCGGUGCUUU,
(SEQ ID NO: 19)
AAACAGCAUAGCAAGUUAAAAUAAGGCUAGUCCGU
UAUCAACUUGAAAAAGUGGCACCGAGUCGGUGCUU
UU,
or
(SEQ ID NO: 20)
GUUGGAACCAUUCAAAACAGCAUAGCAAGUUAAAA
UAAGGCUAGUCCGUUAUCAACUUGAAAAAGUGGCA
CCGAGUCGGUGC.

In systems in which both a crRNA and a tracrRNA are needed, the crRNA and the corresponding tracrRNA hybridize to form a gRNA. In systems in which only a crRNA is needed, the crRNA can be the gRNA. The crRNA additionally provides the single-stranded DNA-targeting segment that hybridizes to the complementary strand of a target DNA. If used for modification within a cell, the exact sequence of a given crRNA or tracrRNA molecule can be designed to be specific to the species in which the RNA molecules will be used. See, e.g., Mali et al. (2013) Science 339(6121):823-826; Jinek et al. (2012) Science 337(6096):816-821; Hwang et al. (2013) Nat. Biotechnol. 31(3):227-229; Jiang et al. (2013) Nat. Biotechnol. 31(3):233-239; and Cong et al. (2013) Science 339(6121):819-823, each of which is herein incorporated by reference in its entirety for all purposes.

The DNA-targeting segment (crRNA) of a given gRNA comprises a nucleotide sequence that is complementary to a sequence on the complementary strand of the target DNA, as described in more detail below. The DNA-targeting segment of a gRNA interacts with the target DNA in a sequence-specific manner via hybridization (i.e., base pairing). As such, the nucleotide sequence of the DNA-targeting segment may vary and determines the location within the target DNA with which the gRNA and the target DNA will interact. The DNA-targeting segment of a subject gRNA can be modified to hybridize to any desired sequence within a target DNA. Naturally occurring crRNAs differ depending on the CRISPR/Cas system and organism but often contain a targeting segment of between 21 to 72 nucleotides length, flanked by two direct repeats (DR) of a length of between 21 to 46 nucleotides (see, e.g., WO 2014/131833, herein incorporated by reference in its entirety for all purposes). In the case of S. pyogenes, the DRs are 36 nucleotides long and the targeting segment is 30 nucleotides long. The 3′ located DR is complementary to and hybridizes with the corresponding tracrRNA, which in turn binds to the Cas protein.

The DNA-targeting segment can have, for example, a length of at least about 12, at least about 15, at least about 17, at least about 18, at least about 19, at least about 20, at least about 25, at least about 30, at least about 35, or at least about 40 nucleotides. Such DNA-targeting segments can have, for example, a length from about 12 to about 100, from about 12 to about 80, from about 12 to about 50, from about 12 to about 40, from about 12 to about 30, from about 12 to about 25, or from about 12 to about 20 nucleotides. For example, the DNA targeting segment can be from about 15 to about 25 nucleotides (e.g., from about 17 to about 20 nucleotides, or about 17, 18, 19, or 20 nucleotides). See, e.g., US 2016/0024523, herein incorporated by reference in its entirety for all purposes. For Cas9 from S. pyogenes, a typical DNA-targeting segment is between 16 and 20 nucleotides in length or between 17 and 20 nucleotides in length. For Cas9 from S. aureus, a typical DNA-targeting segment is between 21 and 23 nucleotides in length. For Cpf1, a typical DNA-targeting segment is at least 16 nucleotides in length or at least 18 nucleotides in length.

In one example, the DNA-targeting segment can be about 20 nucleotides in length. However, shorter and longer sequences can also be used for the targeting segment (e.g., 15-25 nucleotides in length, such as 15, 16, 17, 18, 19, 20, 21, 22, 23, 24, or 25 nucleotides in length). The degree of identity between the DNA-targeting segment and the corresponding guide RNA target sequence (or degree of complementarity between the DNA-targeting segment and the other strand of the guide RNA target sequence) can be, for example, about 75%, about 80%, about 85%, about 90%, about 95%, about 96%, about 97%, about 98%, about 99%, or about 100%. The DNA-targeting segment and the corresponding guide RNA target sequence can contain one or more mismatches. For example, the DNA-targeting segment of the guide RNA and the corresponding guide RNA target sequence can contain 1-4, 1-3, 1-2, 1, 2, 3, or 4 mismatches (e.g., where the total length of the guide RNA target sequence is at least 17, at least 18, at least 19, or at least 20 or more nucleotides). For example, the DNA-targeting segment of the guide RNA and the corresponding guide RNA target sequence can contain 1-4, 1-3, 1-2, 1, 2, 3, or 4 mismatches where the total length of the guide RNA target sequence 20 nucleotides.

As one example, a guide RNA targeting intron 1 of a human ALB gene can comprise a DNA-targeting segment (i.e., guide sequence) comprising, consisting essentially of, or consisting of the sequence (DNA-targeting segment) set forth in any one of SEQ ID NOS: 30-61. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 30-61. Alternatively, a guide RNA targeting intron 1 of a human ALB gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to the sequence (DNA-targeting segment) set forth in any one of SEQ ID NOS: 30-61. Alternatively, a guide RNA targeting intron 1 of a human ALB gene can comprise a DNA-targeting segment that is at least 90%, at least 91%, at least 92%, at least 93%, at least 94%, at least 95%, at least 96%, at least 97%, at least 98%, or at least 99% identical to the sequence (DNA-targeting segment) set forth in any one of SEQ ID NOS: 30-61. Alternatively, a guide RNA targeting intron 1 of a human ALB gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in any one of SEQ ID NOS: 30-61. Alternatively, a guide RNA targeting intron 1 of a human ALB gene can comprise a DNA-targeting segment that is at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in any one of SEQ ID NOS: 30-61. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 30-61. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 30-61.

As another example, a guide RNA targeting intron 1 of a human ALB gene can comprise a DNA-targeting segment (i.e., guide sequence) comprising, consisting essentially of, or consisting of the sequence (DNA-targeting segment) set forth in any one of SEQ ID NOS: 36, 30, 33, and 41. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 36, 30, 33, and 41. Alternatively, a guide RNA targeting intron 1 of a human ALB gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to the sequence (DNA-targeting segment) set forth in any one of SEQ ID NOS: 36, 30, 33, and 41. Alternatively, a guide RNA targeting intron 1 of a human ALB gene can comprise a DNA-targeting segment that is at least 90%, at least 91%, at least 92%, at least 93%, at least 94%, at least 95%, at least 96%, at least 97%, at least 98%, or at least 99% identical to the sequence (DNA-targeting segment) set forth in any one of SEQ ID NOS: 36, 30, 33, and 41. Alternatively, a guide RNA targeting intron 1 of a human ALB gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in any one of SEQ ID NOS: 36, 30, 33, and 41. Alternatively, a guide RNA targeting intron 1 of a human ALB gene can comprise a DNA-targeting segment that is at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in any one of SEQ ID NOS: 36, 30, 33, and 41. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 36, 30, 33, and 41. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 36, 30, 33, and 41.

As another example, a guide RNA targeting intron 1 of a human ALB gene can comprise a DNA-targeting segment (i.e., guide sequence) comprising, consisting essentially of, or consisting of the sequence (DNA-targeting segment) set forth in SEQ ID NO: 36. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 36. Alternatively, a guide RNA targeting intron 1 of a human ALB gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to the sequence (DNA-targeting segment) set forth in SEQ ID NO: 36. Alternatively, a guide RNA targeting intron 1 of a human ALB gene can comprise a DNA-targeting segment that is at least 90%, at least 91%, at least 92%, at least 93%, at least 94%, at least 95%, at least 96%, at least 97%, at least 98%, or at least 99% identical to the sequence (DNA-targeting segment) set forth in SEQ ID NO: 36. Alternatively, a guide RNA targeting intron 1 of a human ALB gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in SEQ ID NO: 36. Alternatively, a guide RNA targeting intron 1 of a human ALB gene can comprise a DNA-targeting segment that is at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in SEQ ID NO: 36. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 36. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 36.

As another example, a guide RNA targeting intron 1 of a human ALB gene can comprise a DNA-targeting segment (i.e., guide sequence) comprising, consisting essentially of, or consisting of the sequence (DNA-targeting segment) set forth in SEQ ID NO: 30. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 30. Alternatively, a guide RNA targeting intron 1 of a human ALB gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to the sequence (DNA-targeting segment) set forth in SEQ ID NO: 30. Alternatively, a guide RNA targeting intron 1 of a human ALB gene can comprise a DNA-targeting segment that is at least 90%, at least 91%, at least 92%, at least 93%, at least 94%, at least 95%, at least 96%, at least 97%, at least 98%, or at least 99% identical to the sequence (DNA-targeting segment) set forth in SEQ ID NO: 30. Alternatively, a guide RNA targeting intron 1 of a human ALB gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in SEQ ID NO: 30. Alternatively, a guide RNA targeting intron 1 of a human ALB gene can comprise a DNA-targeting segment that is at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in SEQ ID NO: 30. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 30. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 30.

As another example, a guide RNA targeting intron 1 of a human ALB gene can comprise a DNA-targeting segment (i.e., guide sequence) comprising, consisting essentially of, or consisting of the sequence (DNA-targeting segment) set forth in SEQ ID NO: 33. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 33. Alternatively, a guide RNA targeting intron 1 of a human ALB gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to the sequence (DNA-targeting segment) set forth in SEQ ID NO: 33. Alternatively, a guide RNA targeting intron 1 of a human ALB gene can comprise a DNA-targeting segment that is at least 90%, at least 91%, at least 92%, at least 93%, at least 94%, at least 95%, at least 96%, at least 97%, at least 98%, or at least 99% identical to the sequence (DNA-targeting segment) set forth in SEQ ID NO: 33. Alternatively, a guide RNA targeting intron 1 of a human ALB gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in SEQ ID NO: 33. Alternatively, a guide RNA targeting intron 1 of a human ALB gene can comprise a DNA-targeting segment that is at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in SEQ ID NO: 33. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 33. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 33.

As another example, a guide RNA targeting intron 1 of a human ALB gene can comprise a DNA-targeting segment (i.e., guide sequence) comprising, consisting essentially of, or consisting of the sequence (DNA-targeting segment) set forth in SEQ ID NO: 41. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 41. Alternatively, a guide RNA targeting intron 1 of a human ALB gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to the sequence (DNA-targeting segment) set forth in SEQ ID NO: 41. Alternatively, a guide RNA targeting intron 1 of a human ALB gene can comprise a DNA-targeting segment that is at least 90%, at least 91%, at least 92%, at least 93%, at least 94%, at least 95%, at least 96%, at least 97%, at least 98%, or at least 99% identical to the sequence (DNA-targeting segment) set forth in SEQ ID NO: 41. Alternatively, a guide RNA targeting intron 1 of a human ALB gene can comprise a DNA-targeting segment that is at least 75%, at least 80%, at least 85%, at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in SEQ ID NO: 41. Alternatively, a guide RNA targeting intron 1 of a human ALB gene can comprise a DNA-targeting segment that is at least 90%, at least 91%, at least 92%, at least 93%, at least 94%, at least 95%, at least 96%, at least 97%, at least 98%, or at least 99% identical to at least 17, at least 18, at least 19, or at least 20 contiguous nucleotides of the sequence (DNA-targeting segment) set forth in SEQ ID NO: 41. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 41. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 41.

TABLE 2
Human ALB Intron 1 Guide Sequences.

| Guide Sequence | SEQ ID NO: |
|---|---|
| GAGCAACCUCACUCUUGUCU | 30 |
| AUGCAUUUGUUUCAAAAUAU | 31 |
| UGCAUUUGUUUCAAAAUAUU | 32 |
| AUUUAUGAGAUCAACAGCAC | 33 |
| GAUCAACAGCACAGGUUUUG | 34 |
| UUAAAUAAAGCAUAGUGCAA | 35 |
| UAAAGCAUAGUGCAAUGGAU | 36 |
| UAGUGCAAUGGAUAGGUCUU | 37 |
| UACUAAAACUUUAUUUUACU | 38 |
| AAAGUUGAACAAUAGAAAAA | 39 |
| AAUGCAUAAUCUAAGUCAAA | 40 |
| UAAUAAAAUUCAAACAUCCU | 41 |
| GCAUCUUUAAAGAAUUAUUU | 42 |
| UUUGGCAUUUAUUUCUAAAA | 43 |
| UGUAUUUGUGAAGUCUUACA | 44 |
| UCCUAGGUAAAAAAAAAAAA | 45 |
| UAAUUUUCUUUUGCGCACUA | 46 |
| UGACUGAAACUUCACAGAAU | 47 |
| GACUGAAACUUCACAGAAUA | 48 |
| UUCAUUUUAGUCUGUCUUCU | 49 |
| AUUAUCUAAGUUUGAAUAUA | 50 |
| AAUUUUUAAAAUAGUAUUCU | 51 |
| UGAAUUAUUCUUCUGUUUAA | 52 |
| AUCAUCCUGAGUUUUUCUGU | 53 |
| UUACUAAAACUUUAUUUUAC | 54 |
| ACCUUUUUUUUUUUUUACCU | 55 |
| AGUGCAAUGGAUAGGUCUUU | 56 |
| UGAUUCCUACAGAAAAACUC | 57 |
| UGGGCAAGGGAAGAAAAAAA | 58 |
| CCUCACUCUUGUCUGGGCAA | 59 |
| ACCUCACUCUUGUCUGGGCA | 60 |
| UGAGCAACCUCACUCUUGUC | 61 |

TABLE 3
Human ALB Intron 1 sgRNA Sequences.

| Full Sequence | Full Sequence Modified |
|---|---|
| GAGCAACCUCACUCUUGUCUGUUU | mG*mA*mG*CAACCUCACUCUUGUCUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 62) | U*mU*mU (SEQ ID NO: 94) |
| AUGCAUUUGUUUCAAAAUAUGUUU | mA*mU*mG*CAUUUGUUUCAAAAUAUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 63) | U*mU*mU (SEQ ID NO: 95) |
| UGCAUUUGUUUCAAAAUAUUGUUU | mU*mG*mC*AUUUGUUUCAAAAUAUUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 64) | U*mU*mU (SEQ ID NO: 96) |
| AUUUAUGAGAUCAACAGCACGUUU | mA*mU*mU*UAUGAGAUCAACAGCACGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 65) | U*mU*mU (SEQ ID NO: 97) |
| GAUCAACAGCACAGGUUUUGGUUU | mG*mA*mU*CAACAGCACAGGUUUUGGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 66) | U*mU*mU (SEQ ID NO: 98) |
| UUAAAUAAAGCAUAGUGCAAGUUU | mU*mU*mA*AAUAAAGCAUAGUGCAAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 67) | U*mU*mU (SEQ ID NO: 99) |
| UAAAGCAUAGUGCAAUGGAUGUUU | mU*mA*mA*AGCAUAGUGCAAUGGAUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 68) | U*mU*mU (SEQ ID NO: 100) |
| UAGUGCAAUGGAUAGGUCUUGUUU | mU*mA*mG*UGCAAUGGAUAGGUCUUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 69) | U*mU*mU (SEQ ID NO: 101) |
| UACUAAAACUUUAUUUUACUGUUU | mU*mA*mC*UAAAACUUUAUUUUACUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 70) | U*mU*mU (SEQ ID NO: 102) |
| AAAGUUGAACAAUAGAAAAAGUUU | mA*mA*mA*GUUGAACAAUAGAAAAAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 71) | U*mU*mU (SEQ ID NO: 103) |
| AAUGCAUAAUCUAAGUCAAAGUUU | mA*mA*mU*GCAUAAUCUAAGUCAAAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 72) | U*mU*mU (SEQ ID NO: 104) |
| UAAUAAAAUUCAAACAUCCUGUUU | mU*mA*mA*UAAAAUUCAAACAUCCUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 73) | U*mU*mU (SEQ ID NO: 105) |
| GCAUCUUUAAAGAAUUAUUUGUUU | mG*mC*mA*UCUUUAAAGAAUUAUUUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 74) | U*mU*mU (SEQ ID NO: 106) |
| UUUGGCAUUUAUUUCUAAAAGUUU | mU*mU*mU*GGCAUUUAUUUCUAAAAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 75) | U*mU*mU (SEQ ID NO: 107) |
| UGUAUUUGUGAAGUCUUACAGUUU | mU*mG*mU*AUUUGUGAAGUCUUACAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 76) | U*mU*mU (SEQ ID NO: 108) |
| UCCUAGGUAAAAAAAAAAAAGUUU | mU*mC*mC*UAGGUAAAAAAAAAAAAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 77) | U*mU*mU (SEQ ID NO: 109) |
| UAAUUUUCUUUUGCGCACUAGUUU | mU*mA*mA*UUUUCUUUUGCGCACUAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 78) | U*mU*mU (SEQ ID NO: 110) |
| UGACUGAAACUUCACAGAAUGUUU | mU*mG*mA*CUGAAACUUCACAGAAUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 79) | U*mU*mU (SEQ ID NO: 111) |
| GACUGAAACUUCACAGAAUAGUUU | mG*mA*mC*UGAAACUUCACAGAAUAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 80) | U*mU*mU (SEQ ID NO: 112) |
| UUCAUUUUAGUCUGUCUUCUGUUU | mU*mU*mC*AUUUUAGUCUGUCUUCUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 81) | U*mU*mU (SEQ ID NO: 113) |
| AUUAUCUAAGUUUGAAUAUAGUUU | mA*mU*mU*AUCUAAGUUUGAAUAUAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 82) | U*mU*mU (SEQ ID NO: 114) |
| AAUUUUUAAAAUAGUAUUCUGUUU | mA*mA*mU*UUUUAAAAUAGUAUUCUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 83) | U*mU*mU (SEQ ID NO: 115) |
| UGAAUUAUUCUUCUGUUUAAGUUU | mU*mG*mA*AUUAUUCUUCUGUUUAAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 84) | U*mU*mU (SEQ ID NO: 116) |
| AUCAUCCUGAGUUUUUCUGUGUUU | mA*mU*mC*AUCCUGAGUUUUUCUGUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 85) | U*mU*mU (SEQ ID NO: 117) |
| UUACUAAAACUUUAUUUUACGUUU | mU*mU*mA*CUAAAACUUUAUUUUACGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 86) | U*mU*mU (SEQ ID NO: 118) |
| ACCUUUUUUUUUUUUUACCUGUUU | mA*mC*mC*UUUUUUUUUUUUUACCUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 87) | U*mU*mU (SEQ ID NO: 119) |
| AGUGCAAUGGAUAGGUCUUUGUUU | mA*mG*mU*GCAAUGGAUAGGUCUUUGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 88) | U*mU*mU (SEQ ID NO: 120) |
| UGAUUCCUACAGAAAAACUCGUUU | mU*mG*mA*UUCCUACAGAAAAACUCGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 89) | U*mU*mU (SEQ ID NO: 121) |
| UGGGCAAGGGAAGAAAAAAAGUUU | mU*mG*mG*GCAAGGGAAGAAAAAAAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 90) | U*mU*mU (SEQ ID NO: 122) |
| CCUCACUCUUGUCUGGGCAAGUUU | mC*mC*mU*CACUCUUGUCUGGGCAAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 91) | U*mU*mU (SEQ ID NO: 123) |
| ACCUCACUCUUGUCUGGGCAGUUU | mA*mC*mC*UCACUCUUGUCUGGGCAGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 92) | U*mU*mU (SEQ ID NO: 124) |
| UGAGCAACCUCACUCUUGUCGUUU | mU*mG*mA*GCAACCUCACUCUUGUCGUUUUAGAmGmC |
| UAGAGCUAGAAAUAGCAAGUUAAA | mUmAmGmAmAmAmUmAmGmCAAGUUAAAAUAAGGCU |
| AUAAGGCUAGUCCGUUAUCAACUU | AGUCCGUUAUCAmAmCmUmUmGmAmAmAmAmAmGmU |
| GAAAAAGUGGCACCGAGUCGGUGC | mGmGmCmAmCmCmGmAmGmUmCmGmGmUmGmCmU*m |
| UUUU (SEQ ID NO: 93) | U*mU*mU (SEQ ID NO: 125) |

TABLE 4
Mouse Alb Intron 1 Guide Sequences.

| Guide Sequence | SEQ ID NO: |
|---|---|
| CACUCUUGUCUGUGGAAACA | 228 |

TABLE 5
Mouse Alb Intron 1 sgRNA Sequences.

| Full Sequence | Full Sequence Modified |
|---|---|
| CACUCUUGUCUGUGG | mC*mA*mC*UCUUGU |
| AAACAGUUUUAGAGC | CUGUGGAAACAGUUU |
| UAGAAAUAGCAAGUU | UAGAmGmCmUmAmGm |
| AAAAUAAGGCUAGUC | AmAmAmUmAmGmCAA |
| CGUUAUCAACUUGAA | GUUAAAAUAAGGCUA |
| AAAGUGGCACCGAGU | GUCCGUUAUCAmAmC |
| CGGUGC | mUmUmGmAmAmAmAm |
| UUUU | AmGmUmGmGmCmAmC |
| (SEQ ID NO: 230) | mCmGmAmGmUmCmGm |
| | GmUmGmCmU*m |
| | U*mU*mU |
| | (SEQ ID NO: 231) |

TracrRNAs can be in any form (e.g., full-length tracrRNAs or active partial tracrRNAs) and of varying lengths. They can include primary transcripts or processed forms. For example, tracrRNAs (as part of a single-guide RNA or as a separate molecule as part of a two-molecule gRNA) may comprise, consist essentially of, or consist of all or a portion of a wild type tracrRNA sequence (e.g., about or more than about 20, 26, 32, 45, 48, 54, 63, 67, 85, or more nucleotides of a wild type tracrRNA sequence). Examples of wild type tracrRNA sequences from S. pyogenes include 171-nucleotide, 89-nucleotide, 75-nucleotide, and 65-nucleotide versions. See, e.g., Deltcheva et al. (2011) Nature 471(7340):602-607; WO 2014/093661, each of which is herein incorporated by reference in its entirety for all purposes. Examples of tracrRNAs within single-guide RNAs (sgRNAs) include the tracrRNA segments found within +48, +54, +67, and +85 versions of sgRNAs, where “+n” indicates that up to the +n nucleotide of wild type tracrRNA is included in the sgRNA. See U.S. Pat. No. 8,697,359, herein incorporated by reference in its entirety for all purposes.

The percent complementarity between the DNA-targeting segment of the guide RNA and the complementary strand of the target DNA can be at least 60% (e.g., at least 65%, at least 70%, at least 75%, at least 80%, at least 85%, at least 90%, at least 95%, at least 97%, at least 98%, at least 99%, or 100%). The percent complementarity between the DNA-targeting segment and the complementary strand of the target DNA can be at least 60% over about 20 contiguous nucleotides. As an example, the percent complementarity between the DNA-targeting segment and the complementary strand of the target DNA can be 100% over the 14 contiguous nucleotides at the 5′ end of the complementary strand of the target DNA and as low as 0% over the remainder. In such a case, the DNA-targeting segment can be considered to be 14 nucleotides in length. As another example, the percent complementarity between the DNA-targeting segment and the complementary strand of the target DNA can be 100% over the seven contiguous nucleotides at the 5′ end of the complementary strand of the target DNA and as low as 0% over the remainder. In such a case, the DNA-targeting segment can be considered to be 7 nucleotides in length. In some guide RNAs, at least 17 nucleotides within the DNA-targeting segment are complementary to the complementary strand of the target DNA. For example, the DNA-targeting segment can be 20 nucleotides in length and can comprise 1, 2, or 3 mismatches with the complementary strand of the target DNA. In one example, the mismatches are not adjacent to the region of the complementary strand corresponding to the protospacer adjacent motif (PAM) sequence (i.e., the reverse complement of the PAM sequence) (e.g., the mismatches are in the 5′ end of the DNA-targeting segment of the guide RNA, or the mismatches are at least 2, 3, 4, 5, 6, 7, 8, 9, 10, 11, 12, 13, 14, 15, 16, 17, 18, or 19 base pairs away from the region of the complementary strand corresponding to the PAM sequence).

The protein-binding segment of a gRNA can comprise two stretches of nucleotides that are complementary to one another. The complementary nucleotides of the protein-binding segment hybridize to form a double-stranded RNA duplex (dsRNA). The protein-binding segment of a subject gRNA interacts with a Cas protein, and the gRNA directs the bound Cas protein to a specific nucleotide sequence within target DNA via the DNA-targeting segment.

Single-guide RNAs can comprise a DNA-targeting segment and a scaffold sequence (i.e., the protein-binding or Cas-binding sequence of the guide RNA). For example, such guide RNAs can have a 5′ DNA-targeting segment joined to a 3′ scaffold sequence. Exemplary scaffold sequences (e.g., for use with S. pyogenes Cas9) comprise, consist essentially of, or consist of:

GUUUUAGAGCUAGAAAUAGCAAGUUAAAAUAAGGCUAGUCCGUUAUCAACUUGA AAAAGUGGCACCGAGUCGGUGCU (version 1; SEQ ID NO: 21);
GUUGGAACCAUUCAAAACAGCAUAGCAAGUUAAAAUAAGGCUAGUCCGUUAUCA ACUUGAAAAAGUGGCACCGAGUCGGUGC (version 2; SEQ ID NO: 22);
GUUUUAGAGCUAGAAAUAGCAAGUUAAAAUAAGGCUAGUCCGUUAUCAACUUGA AAAAGUGGCACCGAGUCGGUGC (version 3; SEQ ID NO: 23); and
GUUUAAGAGCUAUGCUGGAAACAGCAUAGCAAGUUUAAAUAAGGCUAGUCCGUU AUCAACUUGAAAAAGUGGCACCGAGUCGGUGC (version 4; SEQ ID NO: 24);
GUUUUAGAGCUAGAAAUAGCAAGUUAAAAUAAGGCUAGUCCGUUAUCAACUUGA AAAAGUGGCACCGAGUCGGUGCUUUUUUU (version 5; SEQ ID NO: 25);
GUUUUAGAGCUAGAAAUAGCAAGUUAAAAUAAGGCUAGUCCGUUAUCAACUUGA AAAAGUGGCACCGAGUCGGUGCUUUU (version 6; SEQ ID NO: 26);
GUUUAAGAGCUAUGCUGGAAACAGCAUAGCAAGUUUAAAUAAGGCUAGUCCGUU AUCAACUUGAAAAAGUGGCACCGAGUCGGUGCUUUUUU (version 7; SEQ ID NO: 27);
or
GUUUUAGAGCUAGAAAUAGCAAGUUAAAAUAAGGCUAGUCCGUUAUCAACUUGG CACCGAGUCGGUGC (version 8; SEQ ID NO: 28). In some guide sgRNAs, the four terminal U residues of version 6 are not present. In some sgRNAs, only 1, 2, or 3 of the four terminal U residues of version 6 are present. Guide RNAs targeting any of the guide RNA target sequences disclosed herein can include, for example, a DNA-targeting segment on the 5′ end of the guide RNA fused to any of the exemplary guide RNA scaffold sequences on the 3′ end of the guide RNA. That is, any of the DNA-targeting segments disclosed herein can be joined to the 5′ end of any one of the above scaffold sequences to form a single guide RNA (chimeric guide RNA).

Guide RNAs can include modifications or sequences that provide for additional desirable features (e.g., modified or regulated stability; subcellular targeting; tracking with a fluorescent label; a binding site for a protein or protein complex; and the like). That is, guide RNAs can include one or more modified nucleosides or nucleotides, or one or more non-naturally and/or naturally occurring components or configurations that are used instead of or in addition to the canonical A, G, C, and U residues. Examples of such modifications include, for example, a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof. Other examples of modifications include engineered stem loop duplex structures, engineered bulge regions, engineered hairpins 3′ of the stem loop duplex structure, or any combination thereof. See, e.g., US 2015/0376586, herein incorporated by reference in its entirety for all purposes. A bulge can be an unpaired region of nucleotides within the duplex made up of the crRNA-like region and the minimum tracrRNA-like region. A bulge can comprise, on one side of the duplex, an unpaired 5′-XXXY-3′ where X is any purine and Y can be a nucleotide that can form a wobble pair with a nucleotide on the opposite strand, and an unpaired nucleotide region on the other side of the duplex.

Guide RNAs can comprise modified nucleosides and modified nucleotides including, for example, one or more of the following: (1) alteration or replacement of one or both of the non-linking phosphate oxygens and/or of one or more of the linking phosphate oxygens in the phosphodiester backbone linkage (an exemplary backbone modification); (2) alteration or replacement of a constituent of the ribose sugar such as alteration or replacement of the 2′ hydroxyl on the ribose sugar (an exemplary sugar modification); (3) replacement (e.g., wholesale replacement) of the phosphate moiety with dephospho linkers (an exemplary backbone modification); (4) modification or replacement of a naturally occurring nucleobase, including with a non-canonical nucleobase (an exemplary base modification); (5) replacement or modification of the ribose-phosphate backbone (an exemplary backbone modification); (6) modification of the 3′ end or 5′ end of the oligonucleotide (e.g., removal, modification or replacement of a terminal phosphate group or conjugation of a moiety, cap, or linker (such 3′ or 5′ cap modifications may comprise a sugar and/or backbone modification); and (7) modification or replacement of the sugar (an exemplary sugar modification). Other possible guide RNA modifications include modifications of or replacement of uracils or poly-uracil tracts. See, e.g., WO 2015/048577 and US 2016/0237455, each of which is herein incorporated by reference in its entirety for all purposes. Similar modifications can be made to Cas-encoding nucleic acids, such as Cas mRNAs. For example, Cas mRNAs can be modified by depletion of uridine using synonymous codons.

Chemical modifications such at hose listed above can be combined to provide modified gRNAs and/or mRNAs comprising residues (nucleosides and nucleotides) that can have two, three, four, or more modifications. For example, a modified residue can have a modified sugar and a modified nucleobase. In one example, every base of a gRNA is modified (e.g., all bases have a modified phosphate group, such as a phosphorothioate group). For example, all or substantially all of the phosphate groups of a gRNA can be replaced with phosphorothioate groups. Alternatively or additionally, a modified gRNA can comprise at least one modified residue at or near the 5′ end. Alternatively or additionally, a modified gRNA can comprise at least one modified residue at or near the 3′ end.

Some gRNAs comprise one, two, three or more modified residues. For example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of the positions in a modified gRNA can be modified nucleosides or nucleotides.

Unmodified nucleic acids can be prone to degradation. Exogenous nucleic acids can also induce an innate immune response. Modifications can help introduce stability and reduce immunogenicity. Some gRNAs described herein can contain one or more modified nucleosides or nucleotides to introduce stability toward intracellular or serum-based nucleases. Some modified gRNAs described herein can exhibit a reduced innate immune response when introduced into a population of cells.

The gRNAs disclosed herein can comprise a backbone modification in which the phosphate group of a modified residue can be modified by replacing one or more of the oxygens with a different substituent. The modification can include the wholesale replacement of an unmodified phosphate moiety with a modified phosphate group as described herein. Backbone modifications of the phosphate backbone can also include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (Rp) or the “S” configuration (Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group (a sugar modification). For example, the 2′ hydroxyl group (OH) can be modified (e.g., replaced with a number of different oxy or deoxy substituents. Modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). The 2′ hydroxyl group modification can be 2′-O-Me. Likewise, the 2′ hydroxyl group modification can be a 2′-fluoro modification, which replaces the 2′ hydroxyl group with a fluoride. The 2′ hydroxyl group modification can include locked nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). The 2′ hydroxyl group modification can include unlocked nucleic acids (UNA) in which the ribose ring lacks the C2′-C3′ bond. The 2′ hydroxyl group modification can include the methoxyethyl group (MOE), (OCH₂CH₂OCH₃, e.g., a PEG derivative).

Deoxy 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH₂; alkylamino, dialkylamino, heterocyclyl, arylamino, diarylamino, heteroarylamino, diheteroarylamino, or amino acid); NH(CH₂CH₂NH)ₙCH₂CH₂— amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form (e.g. L-nucleosides).

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In a dual guide RNA, each of the crRNA and the tracrRNA can contain modifications. Such modifications may be at one or both ends of the crRNA and/or tracrRNA. In a sgRNA, one or more residues at one or both ends of the sgRNA may be chemically modified, and/or internal nucleosides may be modified, and/or the entire sgRNA may be chemically modified. Some gRNAs comprise a 5′ end modification. Some gRNAs comprise a 3′ end modification.

The guide RNAs disclosed herein can comprise one of the modification patterns disclosed in WO 2018/107028 A1, herein incorporated by reference in its entirety for all purposes. The guide RNAs disclosed herein can also comprise one of the structures/modification patterns disclosed in US 2017/0114334, herein incorporated by reference in its entirety for all purposes. The guide RNAs disclosed herein can also comprise one of the structures/modification patterns disclosed in WO 2017/136794, WO 2017/004279, US 2018/0187186, or US 2019/0048338, each of which is herein incorporated by reference in its entirety for all purposes.

As one example, nucleotides at the 5′ or 3′ end of a guide RNA can include phosphorothioate linkages (e.g., the bases can have a modified phosphate group that is a phosphorothioate group). For example, a guide RNA can include phosphorothioate linkages between the 2, 3, or 4 terminal nucleotides at the 5′ or 3′ end of the guide RNA. As another example, nucleotides at the 5′ and/or 3′ end of a guide RNA can have 2′-O-methyl modifications. For example, a guide RNA can include 2′-O-methyl modifications at the 2, 3, or 4 terminal nucleotides at the 5′ and/or 3′ end of the guide RNA (e.g., the 5′ end). See, e.g., WO 2017/173054 A1 and Finn et al. (2018) Cell Rep. 22(9):2227-2235, each of which is herein incorporated by reference in its entirety for all purposes. Other possible modifications are described in more detail elsewhere herein. In a specific example, a guide RNA includes 2′-O-methyl analogs and 3′ phosphorothioate internucleotide linkages at the first three 5′ and 3′ terminal RNA residues. Such chemical modifications can, for example, provide greater stability and protection from exonucleases to guide RNAs, allowing them to persist within cells for longer than unmodified guide RNAs. Such chemical modifications can also, for example, protect against innate intracellular immune responses that can actively degrade RNA or trigger immune cascades that lead to cell death.

As one example, any of the guide RNAs described herein can comprise at least one modification. In one example, the at least one modification comprises a 2′-O-methyl (2′-O-Me) modified nucleotide, a phosphorothioate (PS) bond between nucleotides, a 2′-fluoro (2′-F) modified nucleotide, or a combination thereof. For example, the at least one modification can comprise a 2′-O-methyl (2′-O-Me) modified nucleotide. Alternatively or additionally, the at least one modification can comprise a phosphorothioate (PS) bond between nucleotides. Alternatively or additionally, the at least one modification can comprise a 2′-fluoro (2′-F) modified nucleotide. In one example, a guide RNA described herein comprises one or more 2′-O-methyl (2′-O-Me) modified nucleotides and one or more phosphorothioate (PS) bonds between nucleotides.

The modifications can occur anywhere in the guide RNA. As one example, the guide RNA comprises a modification at one or more of the first five nucleotides at the 5′ end of the guide RNA, the guide RNA comprises a modification at one or more of the last five nucleotides of the 3′ end of the guide RNA, or a combination thereof. For example, the guide RNA can comprise phosphorothioate bonds between the first four nucleotides of the guide RNA, phosphorothioate bonds between the last four nucleotides of the guide RNA, or a combination thereof. Alternatively or additionally, the guide RNA can comprise 2′-O-Me modified nucleotides at the first three nucleotides at the 5′ end of the guide RNA, can comprise 2′-O-Me modified nucleotides at the last three nucleotides at the 3′ end of the guide RNA, or a combination thereof.

In one example, a modified gRNA can comprise the following sequence: mN*mN*mN*NNNNNNNNNNNNNNNNNGUUUUAGAmGmCmUmAmGmAmAmAmUmA mGmCAAGUUAAAAUAAGGCUAGUCCGUUAUCAmAmCmUmUmGmAmAmAmAmAm GmUmGmGmCmAmCmCmGmAmGmUmCmGmGmUmGmCmU*mU*mU*mU (SEQ ID NO: 29), where “N” may be any natural or non-natural nucleotide. For example, the totality of N residues comprise a human ALB intron 1 DNA-targeting segment as described herein (e.g., the sequence set forth in SEQ ID NO: 29, wherein the N residues are replaced with the DNA-targeting segment of any one of SEQ ID NOS: 30-61, the DNA-targeting segment of any one of SEQ ID NOS: 36, 30, 33, and 41, or the DNA-targeting segment of SEQ ID NO: 36. For example, a modified gRNA can comprise the sequence set forth in any one of SEQ ID NOS: 94-125, the sequence set forth in any one of SEQ ID NOS: 100, 94, 97, and 105, or the sequence set forth in SEQ ID NO: 100 in Table 3. The terms “mA,” “mC,” “mU,” and “mG” denote a nucleotide (A, C, U, and G, respectively) that has been modified with 2′-O-Me. The symbol “*” depicts a phosphorothioate modification. A phosphorothioate linkage or bond refers to a bond where a sulfur is substituted for one nonbridging phosphate oxygen in a phosphodiester linkage, for example in the bonds between nucleotides bases. When phosphorothioates are used to generate oligonucleotides, the modified oligonucleotides may also be referred to as S-oligos. The terms A*, C*, U*, or G* denote a nucleotide that is linked to the next (e.g., 3′) nucleotide with a phosphorothioate bond. The terms “mA*,” “mC*,” “mU*,” and “mG*” denote a nucleotide (A, C, U, and G, respectively) that has been substituted with 2′-O-Me and that is linked to the next (e.g., 3′) nucleotide with a phosphorothioate bond.

Another chemical modification that has been shown to influence nucleotide sugar rings is halogen substitution. For example, 2′-fluoro (2′-F) substitution on nucleotide sugar rings can increase oligonucleotide binding affinity and nuclease stability. Abasic nucleotides refer to those which lack nitrogenous bases. Inverted bases refer to those with linkages that are inverted from the normal 5′ to 3′ linkage (i.e., either a 5′ to 5′ linkage or a 3′ to 3′ linkage).

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In one example, one or more of the first three, four, or five nucleotides at the 5′ terminus, and one or more of the last three, four, or five nucleotides at the 3′ terminus are modified. The modification can be, for example, a 2′-O-Me, 2′-F, inverted abasic nucleotide, phosphorothioate bond, or other nucleotide modification well known to increase stability and/or performance.

In another example, the first four nucleotides at the 5′ terminus, and the last four nucleotides at the 3′ terminus can be linked with phosphorothioate bonds.

In another example, the first three nucleotides at the 5′ terminus, and the last three nucleotides at the 3′ terminus can comprise a 2′-O-methyl (2′-O-Me) modified nucleotide. In another example, the first three nucleotides at the 5′ terminus, and the last three nucleotides at the 3′ terminus comprise a 2′-fluoro (2′-F) modified nucleotide. In another example, the first three nucleotides at the 5′ terminus, and the last three nucleotides at the 3′ terminus comprise an inverted abasic nucleotide.

Guide RNAs can be provided in any form. For example, the gRNA can be provided in the form of RNA, either as two molecules (separate crRNA and tracrRNA) or as one molecule (sgRNA), and optionally in the form of a complex with a Cas protein. The gRNA can also be provided in the form of DNA encoding the gRNA. The DNA encoding the gRNA can encode a single RNA molecule (sgRNA) or separate RNA molecules (e.g., separate crRNA and tracrRNA). In the latter case, the DNA encoding the gRNA can be provided as one DNA molecule or as separate DNA molecules encoding the crRNA and tracrRNA, respectively.

When a gRNA is provided in the form of DNA, the gRNA can be transiently, conditionally, or constitutively expressed in the cell. DNAs encoding gRNAs can be stably integrated into the genome of the cell and operably linked to a promoter active in the cell. Alternatively, DNAs encoding gRNAs can be operably linked to a promoter in an expression construct. For example, the DNA encoding the gRNA can be in a vector comprising a heterologous nucleic acid, such as a nucleic acid encoding a Cas protein. Alternatively, it can be in a vector or a plasmid that is separate from the vector comprising the nucleic acid encoding the Cas protein. Promoters that can be used in such expression constructs include promoters active, for example, in one or more of a eukaryotic cell, a human cell, a non-human cell, a mammalian cell, a non-human mammalian cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Such promoters can also be, for example, bidirectional promoters. Specific examples of suitable promoters include an RNA polymerase III promoter, such as a human U6 promoter, a rat U6 polymerase III promoter, or a mouse U6 polymerase III promoter.

Alternatively, gRNAs can be prepared by various other methods. For example, gRNAs can be prepared by in vitro transcription using, for example, T7 RNA polymerase (see, e.g., WO 2014/089290 and WO 2014/065596, each of which is herein incorporated by reference in its entirety for all purposes). Guide RNAs can also be a synthetically produced molecule prepared by chemical synthesis. For example, a guide RNA can be chemically synthesized to include 2′-O-methyl analogs and 3′ phosphorothioate internucleotide linkages at the first three 5′ and 3′ terminal RNA residues.

Guide RNAs (or nucleic acids encoding guide RNAs) can be in compositions comprising one or more guide RNAs (e.g., 1, 2, 3, 4, or more guide RNAs) and a carrier increasing the stability of the guide RNA (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules. Such compositions can further comprise a Cas protein, such as a Cas9 protein, or a nucleic acid encoding a Cas protein.

As one example, a guide RNA targeting intron 1 of a human ALB gene can comprise, consist essentially of, or consist of the sequence set forth in any one of SEQ ID NOS: 62-125. Alternatively, a guide RNA targeting intron 1 of a human ALB gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the DNA-targeting segment set forth in any one of SEQ ID NOS: 62-125. Alternatively, a guide RNA targeting intron 1 of a human ALB gene can comprise, consist essentially of, or consist of a sequence that is at least 90%, at least 91%, at least 92%, at least 93%, at least 94%, at least 95%, at least 96%, at least 97%, at least 98%, or at least 99% identical to the DNA-targeting segment set forth in any one of SEQ ID NOS: 62-125. Alternatively, a guide RNA targeting intron 1 of a human ALB gene can comprise, consist essentially of, or consist of a sequence that differs by no more than 3, no more than 2, or no more than 1 nucleotide from the sequence set forth in any one of SEQ ID NOS: 62-125.

As another example, a guide RNA targeting intron 1 of a human ALB gene can comprise, consist essentially of, or consist of the sequence set forth in any one of SEQ ID NOS: 68, 100, 62, 94, 65, 97, 73, and 105. Alternatively, a guide RNA targeting intron 1 of a human ALB gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the DNA-targeting segment set forth in any one of SEQ ID NOS: 68, 100, 62, 94, 65, 97, 73, and 105. Alternatively, a guide RNA targeting intron 1 of a human ALB gene can comprise, consist essentially of, or consist of a sequence that is at least 90%, at least 91%, at least 92%, at least 93%, at least 94%, at least 95%, at least 96%, at least 97%, at least 98%, or at least 99% identical to the DNA-targeting segment set forth in any one of SEQ ID NOS: 68, 100, 62, 94, 65, 97, 73, and 105. Alternatively, a guide RNA targeting intron 1 of a human ALB gene can comprise, consist essentially of, or consist of a sequence that differs by no more than 3, no more than 2, or no more than 1 nucleotide from the sequence set forth in any one of SEQ ID NOS: 68, 100, 62, 94, 65, 97, 73, and 105.

As another example, a guide RNA targeting intron 1 of a human ALB gene can comprise, consist essentially of, or consist of the sequence set forth in SEQ ID NO: 68 or 100. Alternatively, a guide RNA targeting intron 1 of a human ALB gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the DNA-targeting segment set forth in SEQ ID NO: 68 or 100. Alternatively, a guide RNA targeting intron 1 of a human ALB gene can comprise, consist essentially of, or consist of a sequence that is at least 90%, at least 91%, at least 92%, at least 93%, at least 94%, at least 95%, at least 96%, at least 97%, at least 98%, or at least 99% identical to the DNA-targeting segment set forth in SEQ ID NO: 68 or 100. Alternatively, a guide RNA targeting intron 1 of a human ALB gene can comprise, consist essentially of, or consist of a sequence that differs by no more than 3, no more than 2, or no more than 1 nucleotide from the sequence set forth in SEQ ID NO: 68 or 100.

As another example, a guide RNA targeting intron 1 of a human ALB gene can comprise, consist essentially of, or consist of the sequence set forth in SEQ ID NO: 62 or 94. Alternatively, a guide RNA targeting intron 1 of a human ALB gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the DNA-targeting segment set forth in SEQ ID NO: 62 or 94. Alternatively, a guide RNA targeting intron 1 of a human ALB gene can comprise, consist essentially of, or consist of a sequence that is at least 90%, at least 91%, at least 92%, at least 93%, at least 94%, at least 95%, at least 96%, at least 97%, at least 98%, or at least 99% identical to the DNA-targeting segment set forth in SEQ ID NO: 62 or 94. Alternatively, a guide RNA targeting intron 1 of a human ALB gene can comprise, consist essentially of, or consist of a sequence that differs by no more than 3, no more than 2, or no more than 1 nucleotide from the sequence set forth in SEQ ID NO: 62 or 94.

As another example, a guide RNA targeting intron 1 of a human ALB gene can comprise, consist essentially of, or consist of the sequence set forth in SEQ ID NO: 65 or 97. Alternatively, a guide RNA targeting intron 1 of a human ALB gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the DNA-targeting segment set forth in SEQ ID NO: 65 or 97. Alternatively, a guide RNA targeting intron 1 of a human ALB gene can comprise, consist essentially of, or consist of a sequence that is at least 90%, at least 91%, at least 92%, at least 93%, at least 94%, at least 95%, at least 96%, at least 97%, at least 98%, or at least 99% identical to the DNA-targeting segment set forth in SEQ ID NO: 65 or 97. Alternatively, a guide RNA targeting intron 1 of a human ALB gene can comprise, consist essentially of, or consist of a sequence that differs by no more than 3, no more than 2, or no more than 1 nucleotide from the sequence set forth in SEQ ID NO: 65 or 97.

As another example, a guide RNA targeting intron 1 of a human ALB gene can comprise, consist essentially of, or consist of the sequence set forth in SEQ ID NO: 73 or 105. Alternatively, a guide RNA targeting intron 1 of a human ALB gene can comprise, consist essentially of, or consist of a sequence that is at least 75%, at least 80%, at least 85%, at least 90%, at least 91%, at least 92%, at least 93%, at least 94%, at least 95%, at least 96%, at least 97%, at least 98%, or at least 99% identical to the DNA-targeting segment set forth in SEQ ID NO: 73 or 105. Alternatively, a guide RNA targeting intron 1 of a human ALB gene can comprise, consist essentially of, or consist of a sequence that is at least 90%, at least 91%, at least 92%, at least 93%, at least 94%, at least 95%, at least 96%, at least 97%, at least 98%, or at least 99% identical to the DNA-targeting segment set forth in SEQ ID NO: 73 or 105. Alternatively, a guide RNA targeting intron 1 of a human ALB gene can comprise, consist essentially of, or consist of a sequence that differs by no more than 3, no more than 2, or no more than 1 nucleotide from the sequence set forth in SEQ ID NO: 73 or 105.

(4) Guide RNA Target Sequences

Target DNAs for guide RNAs include nucleic acid sequences present in a DNA to which a DNA-targeting segment of a gRNA will bind, provided sufficient conditions for binding exist. Suitable DNA/RNA binding conditions include physiological conditions normally present in a cell. Other suitable DNA/RNA binding conditions (e.g., conditions in a cell-free system) are known in the art (see, e.g., Molecular Cloning: A Laboratory Manual, 3rd Ed. (Sambrook et al., Harbor Laboratory Press 2001), herein incorporated by reference in its entirety for all purposes). The strand of the target DNA that is complementary to and hybridizes with the gRNA can be called the “complementary strand,” and the strand of the target DNA that is complementary to the “complementary strand” (and is therefore not complementary to the Cas protein or gRNA) can be called “noncomplementary strand” or “template strand.”

The target DNA includes both the sequence on the complementary strand to which the guide RNA hybridizes and the corresponding sequence on the non-complementary strand (e.g., adjacent to the protospacer adjacent motif (PAM)). The term “guide RNA target sequence” as used herein refers specifically to the sequence on the non-complementary strand corresponding to (i.e., the reverse complement of) the sequence to which the guide RNA hybridizes on the complementary strand. That is, the guide RNA target sequence refers to the sequence on the non-complementary strand adjacent to the PAM (e.g., upstream or 5′ of the PAM in the case of Cas9). A guide RNA target sequence is equivalent to the DNA-targeting segment of a guide RNA, but with thymines instead of uracils. As one example, a guide RNA target sequence for an SpCas9 enzyme can refer to the sequence upstream of the 5′-NGG-3′ PAM on the non-complementary strand. A guide RNA is designed to have complementarity to the complementary strand of a target DNA, where hybridization between the DNA-targeting segment of the guide RNA and the complementary strand of the target DNA promotes the formation of a CRISPR complex. Full complementarity is not necessarily required, provided that there is sufficient complementarity to cause hybridization and promote formation of a CRISPR complex. If a guide RNA is referred to herein as targeting a guide RNA target sequence, what is meant is that the guide RNA hybridizes to the complementary strand sequence of the target DNA that is the reverse complement of the guide RNA target sequence on the non-complementary strand.

A target DNA or guide RNA target sequence can comprise any polynucleotide, and can be located, for example, in the nucleus or cytoplasm of a cell or within an organelle of a cell, such as a mitochondrion or chloroplast. A target DNA or guide RNA target sequence can be any nucleic acid sequence endogenous or exogenous to a cell. The guide RNA target sequence can be a sequence coding a gene product (e.g., a protein) or a non-coding sequence (e.g., a regulatory sequence) or can include both.

Site-specific binding and cleavage of a target DNA by a Cas protein can occur at locations determined by both (i) base-pairing complementarity between the guide RNA and the complementary strand of the target DNA and (ii) a short motif, called the protospacer adjacent motif (PAM), in the non-complementary strand of the target DNA. The PAM can flank the guide RNA target sequence. Optionally, the guide RNA target sequence can be flanked on the 3′ end by the PAM (e.g., for Cas9). Alternatively, the guide RNA target sequence can be flanked on the 5′ end by the PAM (e.g., for Cpf1). For example, the cleavage site of Cas proteins can be about 1 to about 10 or about 2 to about 5 base pairs (e.g., 3 base pairs) upstream or downstream of the PAM sequence (e.g., within the guide RNA target sequence). In the case of SpCas9, the PAM sequence (i.e., on the non-complementary strand) can be 5′-N₁GG-3′, where N₁ is any DNA nucleotide, and where the PAM is immediately 3′ of the guide RNA target sequence on the non-complementary strand of the target DNA. As such, the sequence corresponding to the PAM on the complementary strand (i.e., the reverse complement) would be 5′-CCN₂-3′, where N2 is any DNA nucleotide and is immediately 5′ of the sequence to which the DNA-targeting segment of the guide RNA hybridizes on the complementary strand of the target DNA. In some such cases, N₁ and N₂ can be complementary and the N₁-N₂ base pair can be any base pair (e.g., N₁=C and N₂=G; N₁=G and N₂=C; N₁=A and N₂=T; or N₁=T, and N₂=A). In the case of Cas9 from S. aureus, the PAM can be NNGRRT or NNGRR, where N can A, G, C, or T, and R can be G or A. In the case of Cas9 from C. jejuni, the PAM can be, for example, NNNNACAC or NNNNRYAC, where N can be A, G, C, or T, and R can be G or A. In some cases (e.g., for FnCpf1), the PAM sequence can be upstream of the 5′ end and have the sequence 5′-TTN-3′. In the case of DpbCasX, the PAM can have the sequence 5′-TTCN-3′. In the case of CasΦ, the PAM can have the sequence 5′-TBN-3′, wherein B is G, T, or C.

An example of a guide RNA target sequence is a 20-nucleotide DNA sequence immediately preceding an NGG motif recognized by an SpCas9 protein. For example, two examples of guide RNA target sequences plus PAMs are GN₁₉NGG (SEQ ID NO: 5) or N₂₀NGG (SEQ ID NO: 6). See, e.g., WO 2014/165825, herein incorporated by reference in its entirety for all purposes. The guanine at the 5′ end can facilitate transcription by RNA polymerase in cells. Other examples of guide RNA target sequences plus PAMs can include two guanine nucleotides at the 5′ end (e.g., GGN₂₀NGG; SEQ ID NO: 7) to facilitate efficient transcription by T7 polymerase in vitro. See, e.g., WO 2014/065596, herein incorporated by reference in its entirety for all purposes. Other guide RNA target sequences plus PAMs can have between 4-22 nucleotides in length of SEQ ID NOS: 5-7, including the 5′ G or GG and the 3′ GG or NGG. Yet other guide RNA target sequences plus PAMs can have between 14 and 20 nucleotides in length of SEQ ID NOS: 5-7.

Formation of a CRISPR complex hybridized to a target DNA can result in cleavage of one or both strands of the target DNA within or near the region corresponding to the guide RNA target sequence (i.e., the guide RNA target sequence on the non-complementary strand of the target DNA and the reverse complement on the complementary strand to which the guide RNA hybridizes). For example, the cleavage site can be within the guide RNA target sequence (e.g., at a defined location relative to the PAM sequence). The “cleavage site” includes the position of a target DNA at which a Cas protein produces a single-strand break or a double-strand break. The cleavage site can be on only one strand (e.g., when a nickase is used) or on both strands of a double-stranded DNA. Cleavage sites can be at the same position on both strands (producing blunt ends; e.g. Cas9)) or can be at different sites on each strand (producing staggered ends (i.e., overhangs); e.g., Cpf1). Staggered ends can be produced, for example, by using two Cas proteins, each of which produces a single-strand break at a different cleavage site on a different strand, thereby producing a double-strand break. For example, a first nickase can create a single-strand break on the first strand of double-stranded DNA (dsDNA), and a second nickase can create a single-strand break on the second strand of dsDNA such that overhanging sequences are created. In some cases, the guide RNA target sequence or cleavage site of the nickase on the first strand is separated from the guide RNA target sequence or cleavage site of the nickase on the second strand by at least 2, 3, 4, 5, 6, 7, 8, 9, 10, 15, 20, 25, 30, 40, 50, 75, 100, 250, 500, or 1,000 base pairs.

The guide RNA target sequence can also be selected to minimize off-target modification or avoid off-target effects (e.g., by avoiding two or fewer mismatches to off-target genomic sequences).

As one example, a guide RNA targeting intron 1 of a human ALB gene can target the guide RNA target sequence set forth in any one of SEQ ID NOS: 126-157. As another example, a guide RNA targeting intron 1 of a human ALB gene can target at least 17, at least 18, at least 19, or at least 20 contiguous nucleotides of the guide RNA target sequence set forth in any one of SEQ ID NOS: 126-157.

As another example, a guide RNA targeting intron 1 of a human ALB gene can target the guide RNA target sequence set forth in any one of SEQ ID NOS: 132, 126, 129, and 137. As another example, a guide RNA targeting intron 1 of a human ALB gene can target at least 17, at least 18, at least 19, or at least 20 contiguous nucleotides of the guide RNA target sequence set forth in any one of SEQ ID NOS: 132, 126, 129, and 137.

As another example, a guide RNA targeting intron 1 of a human ALB gene can target the guide RNA target sequence set forth in SEQ ID NO: 132. As another example, a guide RNA targeting intron 1 of a human ALB gene can target at least 17, at least 18, at least 19, or at least 20 contiguous nucleotides of the guide RNA target sequence set forth in SEQ ID NO: 132.

As another example, a guide RNA targeting intron 1 of a human ALB gene can target the guide RNA target sequence set forth in SEQ ID NO: 126. As another example, a guide RNA targeting intron 1 of a human ALB gene can target at least 17, at least 18, at least 19, or at least 20 contiguous nucleotides of the guide RNA target sequence set forth in SEQ ID NO: 126.

As another example, a guide RNA targeting intron 1 of a human ALB gene can target the guide RNA target sequence set forth in SEQ ID NO: 129. As another example, a guide RNA targeting intron 1 of a human ALB gene can target at least 17, at least 18, at least 19, or at least 20 contiguous nucleotides of the guide RNA target sequence set forth in SEQ ID NO: 129.

As another example, a guide RNA targeting intron 1 of a human ALB gene can target the guide RNA target sequence set forth in SEQ ID NO: 137. As another example, a guide RNA targeting intron 1 of a human ALB gene can target at least 17, at least 18, at least 19, or at least 20 contiguous nucleotides of the guide RNA target sequence set forth in SEQ ID NO: 137.

TABLE 6
Human ALB Intron 1 Guide RNA
Target Sequences.

| Guide RNA Target Sequence | SEQ ID NO: |
|---|---|
| GAGCAACCTCACTCTTGTCT | 126 |
| ATGCATTTGTTTCAAAATAT | 127 |
| TGCATTTGTTTCAAAATATT | 128 |
| ATTTATGAGATCAACAGCAC | 129 |
| GATCAACAGCACAGGTTTTG | 130 |
| TTAAATAAAGCATAGTGCAA | 131 |
| TAAAGCATAGTGCAATGGAT | 132 |
| TAGTGCAATGGATAGGTCTT | 133 |
| TACTAAAACTTTATTTTACT | 134 |
| AAAGTTGAACAATAGAAAAA | 135 |
| AATGCATAATCTAAGTCAAA | 136 |
| TAATAAAATTCAAACATCCT | 137 |
| GCATCTTTAAAGAATTATTT | 138 |
| TTTGGCATTTATTTCTAAAA | 139 |
| TGTATTTGTGAAGTCTTACA | 140 |
| TCCTAGGTAAAAAAAAAAAA | 141 |
| TAATTTTCTTTTGCGCACTA | 142 |
| TGACTGAAACTTCACAGAAT | 143 |
| GACTGAAACTTCACAGAATA | 144 |
| TTCATTTTAGTCTGTCTTCT | 145 |
| ATTATCTAAGTTTGAATATA | 146 |
| AATTTTTAAAATAGTATTCT | 147 |
| TGAATTATTCTTCTGTTTAA | 148 |
| ATCATCCTGAGTTTTTCTGT | 149 |
| TTACTAAAACTTTATTTTAC | 150 |
| ACCTTTTTTTTTTTTTACCT | 151 |
| AGTGCAATGGATAGGTCTTT | 152 |
| TGATTCCTACAGAAAAACTC | 153 |
| TGGGCAAGGGAAGAAAAAAA | 154 |
| CCTCACTCTTGTCTGGGCAA | 155 |
| ACCTCACTCTTGTCTGGGCA | 156 |
| TGAGCAACCTCACTCTTGTC | 157 |

TABLE 7
Mouse Alb Intron 1 Guide RNA Target Sequences.

| Guide RNA Target Sequence | SEQ ID NO: |
|---|---|
| CACTCTTGTCTGTGGAAACA | 229 |

(5) Lipid Nanoparticles Comprising Nuclease Agents

Lipid nanoparticles comprising the nuclease agents (e.g., CRISPR/Cas systems) are also provided. The lipid nanoparticles can alternatively or additionally comprise a F9 nucleic acid construct as disclosed herein. For example, the lipid nanoparticles can comprise a nuclease agent (e.g., CRISPR/Cas system), can comprise a F9 nucleic acid construct, or can comprise both a nuclease agent (e.g., a CRISPR/Cas system) and a F9 nucleic acid construct. Regarding CRISPR/Cas systems, the lipid nanoparticles can comprise the Cas protein in any form (e.g., protein, DNA, or mRNA) and/or can comprise the guide RNA(s) in any form (e.g., DNA or RNA). In one example, the lipid nanoparticles comprise the Cas protein in the form of mRNA (e.g., a modified RNA as described herein) and the guide RNA(s) in the form of RNA (e.g., a modified guide RNA as disclosed herein). As another example, the lipid nanoparticles can comprise the Cas protein in the form of protein and the guide RNA(s) in the form of RNA). In a specific example, the guide RNA and the Cas protein are each introduced in the form of RNA via LNP-mediated delivery in the same LNP. As discussed in more detail elsewhere herein, one or more of the RNAs can be modified. For example, guide RNAs can be modified to comprise one or more stabilizing end modifications at the 5′ end and/or the 3′ end. Such modifications can include, for example, one or more phosphorothioate linkages at the 5′ end and/or the 3′ end and/or one or more 2′-O-methyl modifications at the 5′ end and/or the 3′ end. As another example, Cas mRNA modifications can include substitution with pseudouridine (e.g., fully substituted with pseudouridine), 5′ caps, and polyadenylation. As another example, Cas mRNA modifications can include substitution with N1-methyl-pseudouridine (e.g., fully substituted with N1-methyl-pseudouridine), 5′ caps, and polyadenylation. Other modifications are also contemplated as disclosed elsewhere herein. Delivery through such methods can result in transient Cas expression and/or transient presence of the guide RNA, and the biodegradable lipids improve clearance, improve tolerability, and decrease immunogenicity. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1 and WO 2017/173054 A1, each of which is herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as DSPC. In another example, the other components can comprise a helper lipid such as cholesterol, an optional neutral lipid such as DSPC, and a stealth lipid such as S010, 5024, 5027, 5031, or 5033.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22(9):2227-2235 and WO 2017/173054 A1, each of which is herein incorporated by reference in its entirety for all purposes. In certain LNPs, the cargo can include a guide RNA or a nucleic acid encoding a guide RNA. In certain LNPs, the cargo can include an mRNA encoding a Cas nuclease, such as Cas9, and a guide RNA or a nucleic acid encoding a guide RNA. In certain LNPs, the cargo can include a F9 nucleic acid construct as described elsewhere herein. In certain LNPs, the cargo can include an mRNA encoding a Cas nuclease, such as Cas9, a guide RNA or a nucleic acid encoding a guide RNA, and a F9 nucleic acid construct. In some LNPs, the lipid component comprises an amine lipid such as a biodegradable, ionizable lipid. In some instances, the lipid component comprises biodegradable, ionizable lipid, cholesterol, DSPC, and PEG-DMG. For example, Cas9 mRNA and gRNA can be delivered to cells and animals utilizing lipid formulations comprising ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), cholesterol, DSPC, and PEG2k-DMG.

In some examples, the LNPs comprise cationic lipids. In some examples, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., WO 2019/067992, WO 2017/173054, WO 2015/095340, and WO 2014/136086, each of which is herein incorporated by reference in its entirety for all purposes. In some examples, the LNPs comprise molar ratios of a cationic lipid amine to RNA phosphate (N:P) of about 4.5, about 5.0, about 5.5, about 6.0, or about 6.5. In some examples, the terms cationic and ionizable in the context of LNP lipids are interchangeable (e.g., wherein ionizable lipids are cationic depending on the pH).

The lipid for encapsulation and endosomal escape can be a cationic lipid. The lipid can also be a biodegradable lipid, such as a biodegradable ionizable lipid. One example of a suitable lipid is Lipid A or LP01, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Rep. 22(9):2227-2235 and WO 2017/173054 A1, each of which is herein incorporated by reference in its entirety for all purposes. Another example of a suitable lipid is Lipid B, which is ((5-((dimethylamino)methyl)-1,3-phenylene)bis(oxy))bis(octane-8,1-diyl)bis(decanoate), also called ((5-((dimethylamino)methyl)-1,3-phenylene)bis(oxy))bis(octane-8,1-diyl)bis(decanoate). Another example of a suitable lipid is Lipid C, which is 2-((4-(((3-(dimethylamino)propoxy)carbonyl)oxy)hexadecanoyl)oxy)propane-1,3-diyl(9Z,9′Z,12Z,12′Z)-bis(octadeca-9,12-dienoate). Another example of a suitable lipid is Lipid D, which is 3-(((3-(dimethylamino)propoxy)carbonyl)oxy)-13-(octanoyloxy)tridecyl 3-octylundecanoate. Other suitable lipids include heptatriaconta-6,9,28,31-tetraen-19-yl 4-(dimethylamino)butanoate (also known as [(6Z,9Z,28Z,31Z)-heptatriaconta-6,9,28,31-tetraen-19-yl] 4-(dimethylamino)butanoate or Dlin-MC3-DMA (MC3))).

Some such lipids suitable for use in the LNPs described herein are biodegradable in vivo. For example, LNPs comprising such a lipid include those where at least 75% of the lipid is cleared from the plasma within 8, 10, 12, 24, or 48 hours, or 3, 4, 5, 6, 7, or 10 days. As another example, at least 50% of the LNP is cleared from the plasma within 8, 10, 12, 24, or 48 hours, or 3, 4, 5, 6, 7, or 10 days.

Such lipids may be ionizable depending upon the pH of the medium they are in. For example, in a slightly acidic medium, the lipids may be protonated and thus bear a positive charge. Conversely, in a slightly basic medium, such as, for example, blood where pH is approximately 7.35, the lipids may not be protonated and thus bear no charge. In some embodiments, the lipids may be protonated at a pH of at least about 9, 9.5, or 10. The ability of such a lipid to bear a charge is related to its intrinsic pKa. For example, the lipid may, independently, have a pKa in the range of from about 5.8 to about 6.2.

Neutral lipids function to stabilize and improve processing of the LNPs. Examples of suitable neutral lipids include a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine or 1,2-distearoyl-sn-glycero-3-phosphocholine (DSPC), phosphocholine (DOPC), dimyristoylphosphatidylcholine (DMPC), phosphatidylcholine (PLPC), 1,2-diarachidonoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine di stearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, 1-stearoyl-2-oleoyl-sn-glycero-3-phosphocholine (SOPC), and combinations thereof. For example, the neutral phospholipid may be selected from the group consisting of distearoylphosphatidylcholine (DSPC) and dimyristoyl phosphatidyl ethanolamine (DMPE).

Helper lipids include lipids that enhance transfection. The mechanism by which the helper lipid enhances transfection can include enhancing particle stability. In certain cases, the helper lipid can enhance membrane fusogenicity. Helper lipids include steroids, sterols, and alkyl resorcinols. Examples of suitable helper lipids suitable include cholesterol, 5-heptadecylresorcinol, and cholesterol hemisuccinate. In one example, the helper lipid may be cholesterol or cholesterol hemisuccinate.

Stealth lipids include lipids that alter the length of time the nanoparticles can exist in vivo. Stealth lipids may assist in the formulation process by, for example, reducing particle aggregation and controlling particle size. Stealth lipids may modulate pharmacokinetic properties of the LNP. Suitable stealth lipids include lipids having a hydrophilic head group linked to a lipid moiety.

The hydrophilic head group of stealth lipid can comprise, for example, a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids, and poly N-(2-hydroxypropyl)methacrylamide. The term PEG means any polyethylene glycol or other polyalkylene ether polymer. In certain LNP formulations, the PEG, is a PEG-2K, also termed PEG 2000, which has an average molecular weight of about 2,000 daltons. See, e.g., WO 2017/173054 A1, herein incorporated by reference in its entirety for all purposes.

The lipid moiety of the stealth lipid may be derived, for example, from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. The dialkylglycerol or dialkylglycamide group can further comprise one or more substituted alkyl groups.

As one example, the stealth lipid may be selected from PEG-dilauroylglycerol, PEG-dimyristoylglycerol (PEG-DMG), PEG-dipalmitoylglycerol, PEG-di stearoylglycerol (PEG-DSPE), PEG-dilaurylglycamide, PEG-dimyristylglycamide, PEG-dipalmitoylglycamide, and PEG-distearoylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000] (PEG2k-DMPE), or 1,2-dimyristoyl-rac-glycero-3-methylpolyoxyethylene glycol-2000 (PEG2k-DMG), 1,2-distearoyl-sn-glycero-3-phosphoethanolamine-N-[methoxy(polyethylene glycol)-2000] (PEG2k-DSPE), 1,2-distearoyl-sn-glycerol, methoxypoly ethylene glycol (PEG2k-DSG), poly(ethylene glycol)-2000-dimethacrylate (PEG2k-DMA), and 1,2-distearyloxypropyl-3-amine-N-[methoxy(polyethylene glycol)-2000] (PEG2k-DSA). In one particular example, the stealth lipid may be PEG2k-DMG.

In some embodiments, the PEG lipid includes a glycerol group. In some embodiments, the PEG lipid includes a dimyristoylglycerol (DMG) group. In some embodiments, the PEG lipid comprises PEG2k. In some embodiments, the PEG lipid is a PEG-DMG. In some embodiments, the PEG lipid is a PEG2k-DMG. In some embodiments, the PEG lipid is 1,2-dimyristoyl-rac-glycero-3-methoxypolyethylene glycol-2000. In some embodiments, the PEG2k-DMG is 1,2-dimyristoyl-rac-glycero-3-methoxypolyethylene glycol-2000.

The LNPs can comprise different respective molar ratios of the component lipids in the formulation. The mol-% of the CCD lipid may be, for example, from about 30 mol-% to about 60 mol-%, from about 35 mol-% to about 55 mol-%, from about 40 mol-% to about 50 mol-%, from about 42 mol-% to about 47 mol-%, or about 45%. The mol-% of the helper lipid may be, for example, from about 30 mol-% to about 60 mol-%, from about 35 mol-% to about 55 mol-%, from about 40 mol-% to about 50 mol-%, from about 41 mol-% to about 46 mol-%, or about 44 mol-%. The mol-% of the neutral lipid may be, for example, from about 1 mol-% to about 20 mol-%, from about 5 mol-% to about 15 mol-%, from about 7 mol-% to about 12 mol-%, or about 9 mol-%. The mol-% of the stealth lipid may be, for example, from about 1 mol-% to about 10 mol-%, from about 1 mol-% to about 5 mol-%, from about 1 mol-% to about 3 mol-%, about 2 mol-%, or about 1 mol-%.

The LNPs can have different ratios between the positively charged amine groups of the biodegradable lipid (N) and the negatively charged phosphate groups (P) of the nucleic acid to be encapsulated. This may be mathematically represented by the equation N/P. For example, the N/P ratio may be from about 0.5 to about 100, from about 1 to about 50, from about 1 to about 25, from about 1 to about 10, from about 1 to about 7, from about 3 to about 5, from about 4 to about 5, about 4, about 4.5, or about 5. The N/P ratio can also be from about 4 to about 7 or from about 4.5 to about 6. In specific examples, the N/P ratio can be 4.5 or can be 6.

In some LNPs, the cargo can comprise Cas mRNA (e.g., Cas9 mRNA) and gRNA. The Cas mRNA and gRNAs can be in different ratios. For example, the LNP formulation can include a ratio of Cas mRNA to gRNA nucleic acid ranging from about 25:1 to about 1:25, ranging from about 10:1 to about 1:10, ranging from about 5:1 to about 1:5, or about 1:1. Alternatively, the LNP formulation can include a ratio of Cas mRNA to gRNA nucleic acid from about 1:1 to about 1:5, or about 10:1. Alternatively, the LNP formulation can include a ratio of Cas mRNA to gRNA nucleic acid of about 1:10, 25:1, 10:1, 5:1, 3:1, 1:1, 1:3, 1:5, 1:10, or 1:25. Alternatively, the LNP formulation can include a ratio of Cas mRNA to gRNA nucleic acid of from about 2:1 to about 1:2. Alternatively, the LNP formulation can include a ratio of Cas mRNA to gRNA nucleic acid of from about 1:1 to about 1:2. In specific examples, the ratio of Cas mRNA to gRNA can be about 1:1. In specific examples, the ratio of Cas mRNA to gRNA can be about 1:2. In specific examples, the ratio of Cas mRNA to gRNA can be about 2:1.

Exemplary dosing of LNPs includes about 0.1, about 0.25, about 0.3, about 0.5, about 1, about 2, about 3, about 4, about 5, about 6, about 8, or about 10 mg/kg body weight (mpk) or about 0.1 to about 10, about 0.25 to about 10, about 0.3 to about 10, about 0.5 to about 10, about 1 to about 10, about 2 to about 10, about 3 to about 10, about 4 to about 10, about 5 to about 10, about 6 to about 10, about 8 to about 10, about 0.1 to about 8, about 0.1 to about 6, about 0.1 to about 5, about 0.1 to about 4, about 0.1 to about 3, about 0.1 to about 2, about 0.1 to about 1, about 0.1 to about 0.5, about 0.1 to about 0.3, about 0.1 to about 0.25, about 0.25 to about 8, about 0.3 to about 6, about 0.5 to about 5, about 1 to about 5, or about 2 to about 3 mg/kg body weight with respect to total RNA (Cas9 mRNA and gRNA) cargo content. Such LNPs can be administered, for example, intravenously. In one example, LNP doses between about 0.01 mg/kg and about 10 mg/kg, between about 0.1 and about 10 mg/kg, or between about 0.01 and about 0.3 mg/kg can be used. For example, LNP doses of about 0.01, about 0.03, about 0.1, about 0.3, about 1, about 3, or about 10 mg/kg can be used. Additional exemplary dosing of LNPs includes about 0.1, about 0.25, about 0.3, about 0.5, about 1, about 2, about 3, about 4, about 5, about 6, about 8, or about 10 mg/kg (mpk) body weight or about 0.1 to about 10, about 0.25 to about 10, about 0.3 to about 10, about 0.5 to about 10, about 1 to about 10, about 2 to about 10, about 3 to about 10, about 4 to about 10, about 5 to about 10, about 6 to about 10, about 8 to about 10, about 0.1 to about 8, about 0.1 to about 6, about 0.1 to about 5, about 0.1 to about 4, about 0.1 to about 3, about 0.1 to about 2, about 0.1 to about 1, about 0.1 to about 0.5, about 0.1 to about 0.3, about 0.1 to about 0.25, about 0.25 to about 8, about 0.3 to about 6, about 0.5 to about 5, about 1 to about 5, or about 2 to about 3 mg/kg body weight with respect to total RNA (Cas9 mRNA and gRNA) cargo content. Such LNPs can be administered, for example, intravenously. In one example, LNP doses between about 0.01 mg/kg and about 10 mg/kg, between about 0.1 and about 10 mg/kg, or between about 0.01 and about 0.3 mg/kg can be used. For example, LNP doses of about 0.01, about 0.03, about 0.1, about 0.3, about 0.5, about 1, about 2, about 3, or about 10 mg/kg can be used. In another example, LNP doses between about 0.5 and about 10, between about 0.5 and about 5, between about 0.5 and about 3, between about 1 and about 10, between about 1 and about 5, between about 1 and about 3, or between about 1 and about 2 mg/kg can be used. In another example, LNP doses between about 0.5 and about 3, between about 0.5 and about 2.5, between about 0.5 and about 2, between about 0.5 and about 1.5, between about 0.5 and about 1, between about 1 and about 3, between about 1 and about 2.5, between about 1 and about 2, or between about 1 and about 1.5 mg/kg can be used. In another example, an LNP dose of about 1 mg/kg can be used.

In some LNPs, the cargo can comprise a F9 nucleic acid construct and gRNA. The F9 nucleic acid construct and gRNAs can be in different ratios. For example, the LNP formulation can include a ratio of F9 nucleic acid construct to gRNA nucleic acid ranging from about 25:1 to about 1:25, ranging from about 10:1 to about 1:10, ranging from about 5:1 to about 1:5, or about 1:1. Alternatively, the LNP formulation can include a ratio of F9 nucleic acid construct to gRNA nucleic acid from about 1:1 to about 1:5, about 5:1 to about 1:1, about 10:1, or about 1:10. Alternatively, the LNP formulation can include a ratio of F9 nucleic acid construct to gRNA nucleic acid of about 1:10, about 25:1, about 10:1, about 5:1, about 3:1, about 1:1, about 1:3, about 1:5, about 1:10, or about 1:25.

A specific example of a suitable LNP has a nitrogen-to-phosphate (N/P) ratio of about 4.5 and contains biodegradable cationic lipid, cholesterol, DSPC, and PEG2k-DMG in an about 45:44:9:2 molar ratio (about 45:about 44:about 9:about 2). The biodegradable cationic lipid can be (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Rep. 22(9):2227-2235, herein incorporated by reference in its entirety for all purposes. The Cas9 mRNA can be in an about 1:1 (about 1:about 1) ratio by weight to the guide RNA. Another specific example of a suitable LNP contains Dlin-MC3-DMA (MC3), cholesterol, DSPC, and PEG-DMG in an about 50:38.5:10:1.5 molar ratio (about 50:about 38.5:about 10:about 1.5). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Another specific example of a suitable LNP has a nitrogen-to-phosphate (N/P) ratio of about 6 and contains biodegradable cationic lipid, cholesterol, DSPC, and PEG2k-DMG in an about 50:38:9:3 molar ratio (about 50:about 38:about 9:about 3). The biodegradable cationic lipid can be Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 (about 2:about 1) ratio by weight to the guide RNA.

Another specific example of a suitable LNP has a nitrogen-to-phosphate (N/P) ratio of about 3 and contains a cationic lipid, a structural lipid, cholesterol (e.g., cholesterol (ovine) (Avanti 700000)), and PEG2k-DMG (e.g., PEG-DMG 2000 (NOF America-SUNBRIGHT® GM-020(DMG-PEG)) in an about 50:10:38.5:1.5 ratio (about 50:about 10:about 38.5:about 1.5) or an about 47:10:42:1 ratio (about 47:about 10:about 42:about 1). The structural lipid can be, for example, DSPC (e.g., DSPC (Avanti 850365)), SOPC, DOPC, or DOPE. The cationic/ionizable lipid can be, for example, Dlin-MC3-DMA (e.g., Dlin-MC3-DMA (Biofine International)). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Another specific example of a suitable LNP contains Dlin-MC3-DMA, DSPC, cholesterol, and a PEG lipid in an about 45:9:44:2 ratio (about 45:about 9:about 44:about 2). Another specific example of a suitable LNP contains Dlin-MC3-DMA, DOPE, cholesterol, and PEG lipid or PEG DMG in an about 50:10:39:1 ratio (about 50:about 10:about 39:about 1). Another specific example of a suitable LNP has Dlin-MC3-DMA, DSPC, cholesterol, and PEG2k-DMG at an about 55:10:32.5:2.5 ratio (about 55:about 10:about 32.5:about 2.5). Another specific example of a suitable LNP has Dlin-MC3-DMA, DSPC, cholesterol, and PEG-DMG in an about 50:10:38.5:1.5 ratio (about 50:about 10:about 38.5:about 1.5). Another specific example of a suitable LNP has Dlin-MC3-DMA, DSPC, cholesterol, and PEG-DMG in an about 50:10:38.5:1.5 ratio (about 50:about 10:about 38.5:about 1.5). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Other examples of suitable LNPs can be found, e.g., in WO 2019/067992, WO 2020/082042, US 2020/0270617, WO 2020/082041, US 2020/0268906, WO 2020/082046 (see, e.g., pp. 85-86), and US 2020/0289628, each of which is herein incorporated by reference in its entirety for all purposes.

(6) Vectors Comprising Nuclease Agents

The nuclease agents disclosed herein (e.g., ZFN, TALEN, or CRISPR/Cas) can be provided in a vector for expression. A vector can comprise additional sequences such as, for example, replication origins, promoters, and genes encoding antibiotic resistance.

Some vectors may be circular. Alternatively, the vector may be linear. The vector can be in the packaged for delivered via a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

Introduction of nucleic acids can also be accomplished by virus-mediated delivery, such as AAV-mediated delivery or lentivirus-mediated delivery. The vectors can be, for example, viral vectors such as adeno-associated virus (AAV) vectors. The AAV may be any suitable serotype and may be a single-stranded AAV (ssAAV) or a self-complementary AAV (scAAV). Other exemplary viruses/viral vectors include retroviruses, lentiviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression, long-lasting expression (e.g., at least 1 week, 2 weeks, 1 month, 2 months, or 3 months), or permanent expression (e.g., of Cas and/or gRNA). Viral vector may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper virus to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include about 10¹², about 10¹³, about 10¹⁴, about 10¹⁵, and about 10¹⁶ vector genomes (vg)/mL, or between about 10¹² to about 10¹⁶, between about 10¹² to about 10¹⁵, between about 10¹² to about 10¹⁴, between about 10¹² to about 10¹³, between about 10¹³ to about 10¹⁶, between about 10¹⁴ to about 10¹⁶, between about 10¹⁵ to about 10¹⁶, or between about 10¹³ to about 10¹⁵ vg/mL. Other exemplary viral titers (e.g., AAV titers) include about 10¹², about 10¹³, about 10¹⁴, about 10¹⁵, and about 10¹⁶ vector genomes (vg)/kg of body weight, or between about 10¹² to about 10¹⁶, between about 10¹² to about 10¹⁵, between about 10¹² to about 10¹⁴, between about 10¹² to about 10¹³, between about 10¹³ to about 10¹⁶, between about 10¹⁴ to about 10¹⁶, between about 10¹⁵ to about 10¹⁶, or between about 10¹³ to about 10¹⁵ vg/kg of body weight. In one example, the viral titer is between about 10¹³ to about 10¹⁴ vg/mL or vg/kg. In another example, the viral titer is between about 10¹² to about 10¹³ vg/mL or vg/kg (e.g., between about 10¹² to about 10¹³ vg/kg). In another example, the viral titer is between about 10¹² to about 10¹⁴ vg/mL or vg/kg (e.g., between about 10¹² to about 10¹⁴ vg/kg). For example, the viral titer can be between about 1.5E12 to about 1.5E13 vg/kg, can be about 1.5E12 vg/kg, or can be about 1.5E13 vg/kg. In another example, the viral titer is about 2E13 vg/mL or vg/kg.

Adeno-associated viruses (AAVs) are endemic in multiple species including human and non-human primates (NHPs). At least 12 natural serotypes and hundreds of natural variants have been isolated and characterized to date. See, e.g., Li et al. (2020) Nat. Rev. Genet. 21:255-272, herein incorporated by reference in its entirety for all purposes. AAV particles are naturally composed of a non-enveloped icosahedral protein capsid containing a single-stranded DNA (ssDNA) genome. The DNA genome is flanked by two inverted terminal repeats (ITRs) which serve as the viral origins of replication and packaging signals. The rep gene encodes four proteins required for viral replication and packaging whilst the cap gene encodes the three structural capsid subunits which dictate the AAV serotype, and the Assembly Activating Protein (AAP) which promotes virion assembly in some serotypes.

Recombinant AAV (rAAV) is currently one of the most commonly used viral vectors used in gene therapy to treat human diseases by delivering therapeutic transgenes to target cells in vivo. Indeed, rAAV vectors are composed of icosahedral capsids similar to natural AAVs, but rAAV virions do not encapsidate AAV protein-coding or AAV replicating sequences. These viral vectors are non-replicating. The only viral sequences required in rAAV vectors are the two ITRs, which are needed to guide genome replication and packaging during manufacturing of the rAAV vector. rAAV genomes are devoid of AAV rep and cap genes, rendering them non-replicating in vivo. rAAV vectors are produced by expressing rep and cap genes along with additional viral helper proteins in trans, in combination with the intended transgene cassette flanked by AAV ITRs.

In therapeutic rAAV genomes, a gene expression cassette is placed between ITR sequences. Typically, rAAV genome cassettes comprise of a promoter to drive expression of a therapeutic transgene, followed by polyadenylation sequence. The ITRs flanking a rAAV expression cassette are usually derived from AAV2, the first serotype to be isolated and converted into a recombinant viral vector. Since then, most rAAV production methods rely on AAV2 Rep-based packaging systems. See, e.g., Colella et al. (2017) Mol. Ther. Methods Clin. Dev. 8:87-104, herein incorporated by reference in its entirety for all purposes.

Some non-limiting examples of ITRs that can be used include ITRs comprising, consisting essentially of, or consisting of SEQ ID NO: 196, SEQ ID NO: 197, or SEQ ID NO: 198. Other examples of ITRs comprise one or more mutations compared to SEQ ID NO: 196, SEQ ID NO: 197, or SEQ ID NO: 198 and can be at least 90%, at least 91%, at least 92%, at least 93%, at least 94%, at least 95%, at least 96%, at least 97%, at least 98%, or at least 99% identical to SEQ ID NO: 196, SEQ ID NO: 197, or SEQ ID NO: 198. In some rAAV genomes disclosed herein, the nucleic acid encoding the nuclease agent (or component thereof) is flanked on both sides by the same ITR (i.e., the ITR on the 5′ end, and the reverse complement of the ITR on the 3′ end). In one example, the ITR on each end can comprise, consist essentially of, or consist of SEQ ID NO: 196. In another example, the ITR on each end can comprise, consist essentially of, or consist of SEQ ID NO: 197. In one example, the ITR on at least one end comprises, consists essentially of, or consists of SEQ ID NO: 198. In one example, the ITR on the 5′ end comprises, consists essentially of, or consists of SEQ ID NO: 198. In one example, the ITR on the 3′ end comprises, consists essentially of, or consists of SEQ ID NO: 198. In one example, the ITR on each end can comprise, consist essentially of, or consist of SEQ ID NO: 198. In one example, the ITR on at least one end comprises, consists essentially of, or consists of SEQ ID NO: 196. In one example, the ITR on the 5′ end comprises, consists essentially of, or consists of SEQ ID NO: 196. In one example, the ITR on the 3′ end comprises, consists essentially of, or consists of SEQ ID NO: 196. In one example, the ITR on each end can comprise, consist essentially of, or consist of SEQ ID NO: 196. In other rAAV genomes disclosed herein, the nucleic acid encoding the nuclease agent (or component thereof) is flanked by different ITRs on each end. In one example, the ITR on one end comprises, consists essentially of, or consists of SEQ ID NO: 196, and the ITR on the other end comprises, consists essentially of, or consists of SEQ ID NO: 197. In another example, the ITR on one end comprises, consists essentially of, or consists of SEQ ID NO: 196, and the ITR on the other end comprises, consists essentially of, or consists of SEQ ID NO: 198. In one example, the ITR on one end comprises, consists essentially of, or consists of SEQ ID NO: 197, and the ITR on the other end comprises, consists essentially of, or consists of SEQ ID NO: 198.

The specific serotype of a recombinant AAV vector influences its in-vivo tropism to specific tissues. AAV capsid proteins are responsible for mediating attachment and entry into target cells, followed by endosomal escape and trafficking to the nucleus. Thus, the choice of serotype when developing a rAAV vector will influence what cell types and tissues the vector is most likely to bind to and transduce when injected in vivo. Several serotypes of rAAVs, including rAAV8, are capable of transducing the liver when delivered systemically in mice, NHPs and humans. See, e.g., Li et al. (2020) Nat. Rev. Genet. 21:255-272, herein incorporated by reference in its entirety for all purposes.

Once in the nucleus, the ssDNA genome is released from the virion and a complementary DNA strand is synthesized to generate a double-stranded DNA (dsDNA) molecule. Double-stranded AAV genomes naturally circularize via their ITRs and become episomes which will persist extrachromosomally in the nucleus. Therefore, for episomal gene therapy programs, rAAV-delivered rAAV episomes provide long-term, promoter-driven gene expression in non-dividing cells. However, this rAAV-delivered episomal DNA is diluted out as cells divide. In contrast, the gene therapy described herein is based on gene insertion to allow long-term gene expression.

When specific rAAVs comprising specific sequences (e.g., specific bidirectional construct sequences or specific unidirectional construct sequences) are disclosed herein, they are meant to encompass the sequence disclosed or the reverse complement of the sequence. For example, if a bidirectional or unidirectional construct disclosed herein consists of the hypothetical sequence 5′-CTGGACCGA-3′, it is also meant to encompass the reverse complement of that sequence (5′-TCGGTCCAG-3′). Likewise, when rAAVs comprising bidirectional or unidirectional construct elements in a specific 5′ to 3′ order are disclosed herein, they are also meant to encompass the reverse complement of the order of those elements. For example, if an rAAV is disclosed herein that comprises a bidirectional construct that comprises from 5′ to 3′ a first splice acceptor, a first coding sequence, a first terminator, a reverse complement of a second terminator, a reverse complement of a second coding sequence, and a reverse complement of a second splice acceptor, it is also meant to encompass a construct comprising from 5′ to 3′ the second splice acceptor, the second coding sequence, the second terminator, a reverse complement of the first terminator, a reverse complement of the first coding sequence, and a reverse complement of the first splice acceptor. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediate AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. The term AAV includes, for example, AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. A “AAV vector” as used herein refers to an AAV vector comprising a heterologous sequence not of AAV origin (i.e., a nucleic acid sequence heterologous to AAV), typically comprising a sequence encoding a heterologous polypeptide of interest. The construct may comprise an AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV capsid sequence. In general, the heterologous nucleic acid sequence (the transgene) is flanked by at least one, and generally by two, AAV inverted terminal repeat sequences (ITRs). An AAV vector may either be single-stranded (ssAAV) or self-complementary (scAAV). Examples of serotypes for liver tissue include AAV3B, AAVS, AAV6, AAV7, AAV8, AAV9, AAVrh.74, and AAVhu.37, and particularly AAV8. In a specific example, the AAV vector comprising the nucleic acid construct can be recombinant AAV8 (rAAV8). A rAAV8 vector as described herein is one in which the capsid is from AAV8. For example, an AAV vector using ITRs from AAV2 and a capsid of AAV8 is considered herein to be a rAAV8 vector.

Tropism can be further refined through pseudotyping, which is the mixing of a capsid and a genome from different viral serotypes. For example AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell's DNA replication machinery to synthesize the complementary strand of the AAV's single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

The vector (e.g., AAV such as recombinant AAV8) can be formulated, for example, in 10 mM sodium phosphate, 180 mM sodium chloride, and 0.005% poloxamer 188, at pH 7.3.

In certain AAVs, the cargo can include nucleic acids encoding one or more guide RNAs (e.g., DNA encoding a guide RNA, or DNA encoding two or more guide RNAs). In certain AAVs, the cargo can include a nucleic acid (e.g., DNA) encoding a Cas nuclease, such as Cas9, and DNA encoding one or more guide RNAs (e.g., DNA encoding a guide RNA, or DNA encoding two or more guide RNAs). In certain AAVs, the cargo can include a F9 nucleic acid construct. In certain AAVs, the cargo can include a nucleic acid (e.g., DNA) encoding a Cas nuclease, such as Cas9, a DNA encoding a guide RNA (or multiple guide RNAs), and a F9 nucleic acid construct.

For example, Cas or Cas9 and one or more gRNAs (e.g., 1 gRNA or 2 gRNAs or 3 gRNAs or 4 gRNAs) can be delivered via LNP-mediated delivery (e.g., in the form of RNA) or adeno-associated virus (AAV)-mediated delivery (e.g., rAAV8-mediated delivery). For example, a Cas9 mRNA and a gRNA can be delivered via LNP-mediated delivery, or DNA encoding Cas9 and DNA encoding a gRNA can be delivered via AAV-mediated delivery. The Cas or Cas9 and the gRNA(s) can be delivered in a single AAV or via two separate AAVs. For example, a first AAV can carry a Cas or Cas9 expression cassette, and a second AAV can carry a gRNA expression cassette. Similarly, a first AAV can carry a Cas or Cas9 expression cassette, and a second AAV can carry two or more gRNA expression cassettes. Alternatively, a single AAV can carry a Cas or Cas9 expression cassette (e.g., Cas or Cas9 coding sequence operably linked to a promoter) and a gRNA expression cassette (e.g., gRNA coding sequence operably linked to a promoter). Similarly, a single AAV can carry a Cas or Cas9 expression cassette (e.g., Cas or Cas9 coding sequence operably linked to a promoter) and two or more gRNA expression cassettes (e.g., gRNA coding sequences operably linked to promoters). Different promoters can be used to drive expression of the gRNA, such as a U6 promoter or the small tRNA Gln. Likewise, different promoters can be used to drive Cas9 expression. For example, small promoters are used so that the Cas9 coding sequence can fit into an AAV construct. Similarly, small Cas9 proteins (e.g., SaCas9 or CjCas9 are used to maximize the AAV packaging capacity).

C. Cells or Animals or Genomes

Cells or animals (i.e., subjects) comprising any of the above compositions (e.g., F9 nucleic acid constructs, nuclease agents, vectors, lipid nanoparticles, or any combination thereof) are also provided herein. Such cells or animals (or genomes) can be produced by the methods disclosed herein. For example, the cells or animals can comprise any of the F9 nucleic acid constructs described herein, any of the nuclease agents disclosed herein, or both. Such cells or animals (or genomes) can be neonatal cells or animals (or genomes). Alternatively, such cells or animals (or genomes) can be non-neonatal cells or animals (or genomes).

A neonatal subject (e.g., animal) can be a human subject up to or under the age of 1 year (52 weeks), preferably up to or under the age of 24 weeks, more preferably up to or under the age of 12 weeks, more preferably up to or under the age of 8 weeks, and even more preferably up to or under the age of 4 weeks. In certain embodiments, a neonatal human subject is up to 4 weeks of age. In certain embodiments, a neonatal human subject is up to 8 weeks of age. In another embodiment, a neonatal human subject is within 3 weeks after birth. In another embodiment, a neonatal human subject is within 2 weeks after birth. In another embodiment, a neonatal human subject is within 1 week after birth. In another embodiment, a neonatal human subject is within 7 days after birth. In another embodiment, a neonatal human subject is within 6 days after birth. In another embodiment, a neonatal human subject is within 5 days after birth. In another embodiment, a neonatal human subject is within 4 days after birth. In another embodiment, a neonatal human subject is within 3 days after birth. In another embodiment, a neonatal human subject is within 2 days after birth. In another embodiment, a neonatal human subject is within 1 day after birth. The time windows disclosed above are for human subjects and are also meant to cover the corresponding developmental time windows for other animals. As used herein, a “neonatal cell” is a cell of a neonatal subject, and a population of neonatal cells is a population of cells of a neonatal subject.

Neonatal cells can be of any neonatal subject. For example, they can be from a human subject up to or under the age of 1 year (52 weeks), preferably up to or under the age of 24 weeks, more preferably up to or under the age of 12 weeks, more preferably up to or under the age of 8 weeks, and even more preferably up to or under the age of 4 weeks. In certain embodiments, a neonatal human subject is up to 4 weeks of age. In certain embodiments, a neonatal human subject is up to 8 weeks of age. In another embodiment, a neonatal human subject is within 3 weeks after birth. In another embodiment, a neonatal human subject is within 2 weeks after birth. In another embodiment, a neonatal human subject is within 1 week after birth. In another embodiment, a neonatal human subject is within 7 days after birth. In another embodiment, a neonatal human subject is within 6 days after birth. In another embodiment, a neonatal human subject is within 5 days after birth. In another embodiment, a neonatal human subject is within 4 days after birth. In another embodiment, a neonatal human subject is within 3 days after birth. In another embodiment, a neonatal human subject is within 2 days after birth. In another embodiment, a neonatal human subject is within 1 day after birth. The time windows disclosed above are for human subjects and are also meant to cover the corresponding developmental time windows for other animals. As used herein, a “neonatal cell” is a cell of a neonatal subject, and a population of neonatal cells is a population of cells of a neonatal subject.

In some such cells or animals or genomes, the F9 nucleic acid construct can be genomically integrated at a target genomic locus, such as a safe harbor locus (e.g., an ALB locus or a human ALB locus, such as intron 1 of an ALB locus or a human ALB locus). In some such cells, animals, or genomes, the F9 nucleic acid construct is expressed in the cell, animal, or genome. For example, if the F9 nucleic acid construct is integrated into an ALB locus (e.g., intron 1 of a human ALB locus), FIX protein can be expressed from the ALB locus. The FIX coding sequence can be operably linked to an endogenous promoter at the target genomic locus upon integration into the target genomic locus, or it can be operably linked to an exogenous promoter present in the nucleic acid construct. If the nucleic acid construct is a bidirectional nucleic acid construct disclosed herein, the genome, cell, or animal can express the first FIX protein or can express the second FIX protein. In some genomes, cells, or animals, the target genomic locus is an ALB locus. For example, the nucleic acid construct can be genomically integrated in intron 1 of the endogenous ALB locus. Endogenous ALB exon 1 can then splice into the coding sequence for the FIX protein in the nucleic acid construct.

The target genomic locus at which the nucleic acid construct is stably integrated can be heterozygous for the FIX coding sequence from the nucleic acid construct or homozygous for the FIX coding sequence from the nucleic acid construct. A diploid organism has two alleles at each genetic locus. Each pair of alleles represents the genotype of a specific genetic locus. Genotypes are described as homozygous if there are two identical alleles at a particular locus and as heterozygous if the two alleles differ.

The cells, animals, or genomes can be from any suitable species, such as eukaryotic cells or eukaryotes, or mammalian cells or mammals (e.g., non-human mammalian cells or non-human mammals, or human cells or humans). A mammal can be, for example, a non-human mammal, a human, a rodent, a rat, a mouse, or a hamster. Other non-human mammals include, for example, non-human primates, monkeys, apes, cats, dogs, rabbits, horses, bulls, deer, bison, livestock (e.g., bovine species such as cows, steer, and so forth; ovine species such as sheep, goats, and so forth; and porcine species such as pigs and boars). Birds include, for example, chickens, turkeys, ostrich, geese, ducks, and so forth. Domesticated animals and agricultural animals are also included. The term “non-human” excludes humans. Examples include, but are not limited to, human cells/humans, rodent cells/rodents, mouse cells/mice, rat cells/rats, and non-human primate cells/non-human primates. In a specific example, the cell is a human cell or the animal is a human. Likewise, cells can be any suitable type of cell. In a specific example, the cell is a liver cell such as a hepatocyte (e.g., a human liver cell or human hepatocyte).

The cells can be isolated cells (e.g., in vitro), ex vivo cells, or can be in vivo within an animal (i.e., in a subject). The cells can be mitotically competent cells or mitotically-inactive cells, meiotically competent cells or meiotically-inactive cells. Similarly, the cells can also be primary somatic cells or cells that are not a primary somatic cell. Somatic cells include any cell that is not a gamete, germ cell, gametocyte, or undifferentiated stem cell. For example, the cells can be liver cells, such as hepatocytes (e.g., mouse, non-human primate, or human hepatocytes).

The cells provided herein can be normal, healthy cells, or can be diseased or mutant-bearing cells. For example, the cells can have a FIX deficiency or can be from a subject with FIX deficiency or hemophilia (e.g., hemophilia B).

The cells provided herein can be non-dividing cells. A non-dividing cell refers to cells that are terminally differentiated and do not divide, as well as quiescent cells that do not divide but retains the ability to re-enter cell division and proliferation. Liver cells, for example, retain the ability to divide (e.g., when injured or resected), but do not typically divide. During mitotic cell division, homologous recombination is a mechanism by which the genome is protected and double-stranded breaks are repaired. A non-dividing cell can refer to a cell in which homologous recombination (HR) is not the primary mechanism by which double-stranded DNA breaks are repaired in the cell (e.g., as compared to a control dividing cell). A non-dividing cell can refer to a cell in which non-homologous end joining (NHEJ) is the primary mechanism by which double-stranded DNA breaks are repaired in the cell (e.g., as compared to a control dividing cell). Non-dividing cell types have been described in the literature, for example, by active NHEJ double-stranded DNA break repair mechanisms. See, e.g. Iyama, DNA Repair (Amst.) 2013, 12(8): 620-636.

### III. Methods for Introducing, Integrating, or Expressing a F9 Nucleic Acid or for Treatment of Hemophilia B or FIX Deficiency

The nucleic acid constructs and compositions disclosed herein can be used in methods of introducing a F9 nucleic acid into a cell, methods of integration of a F9 nucleic acid into a target genomic locus, methods of expression of FIX in a cell, and in methods of treating hemophilia B or FIX deficiency in a subject. The nucleic acid constructs and compositions disclosed herein can be used in methods of introducing a F9 nucleic acid into a cell or a population of cells or a subject, methods of inserting or integrating a F9 nucleic acid into a target genomic locus, methods of expressing a FIX protein in a cell or a population of cells or a subject, and in methods of treating hemophilia B or FIX deficiency in a subject.

A. Hemophilia B

The compositions disclosed herein (e.g., F9 nucleic acid constructs, or F9 nucleic acid constructs in combination with the nuclease agents (e.g., CRISPR/Cas systems) are useful for the treatment of FIX deficiency or hemophilia B and/or ameliorating at least one symptom associated with FIX deficiency or hemophilia B. Likewise, the compositions disclosed herein can be used for the preparation of a pharmaceutical composition or medicament for treating a subject having FIX deficiency or hemophilia B.

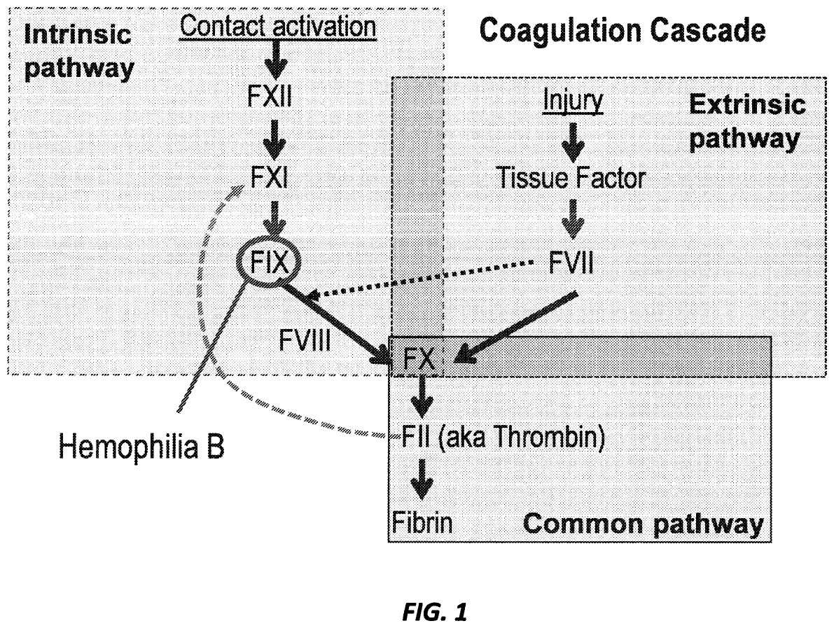

Symptoms and Cause. Hemostasis is a balance between procoagulant and anticoagulant activity to maintain blood in a fluid state under normal conditions and rapidly form a blood clot in the case of a vessel injury. Coagulation is initiated by disruption of the endothelium exposing platelets to collagen and extravascular tissue factor. This results in an activation cascade of clotting factors, including FIX, amplifying the coagulation reaction until activation and polymerization of fibrin monomers form a plug to block blood flow and achieve hemostasis (FIG. 1). The coagulation process is accompanied by clot containment, wound healing, clot dissolution, tissue regeneration and remodeling.

Hemophilia B is a rare congenital disorder caused by an inherited or spontaneous recessive mutation in the F9 gene present on the X chromosome, leading to the expression of a malfunctioning or deficient FIX protein. The absence of a functioning FIX protein interrupts the coagulation cascade and greatly limits the normal formation of blood clots, leading to abnormal bleeding. Due to the recessive nature of the variant, hemophilia usually only affects men, although female carriers may experience mild to moderate symptoms and may require treatment.

Hemophilia is usually inherited from the maternal X chromosome, but prospective studies report that 43% of people newly diagnosed with severe hemophilia B have no prior family history of hemophilia. See, e.g., Srivastava et al. (2020) Haemophilia 26.6:1-158 and Kasper et al. (2007) Haemophilia 13:90-92, each of which is herein incorporated by reference in its entirety for all purposes. The report on the Annual Global Survey conducted in 2019 by the World Federation of Hemophilia reported that 31,997 patients lived with Hemophilia B in the world, with 4,093 patients living in the U.S. See, e.g., World Federation of Hemophilia, “Report on the Annual Global Survey 2019” World Federation of Hemophilia (2020), herein incorporated by reference in its entirety for all purposes. The estimated incidence (prevalence at birth) of hemophilia B worldwide is 5.0 cases per 100,000 males, and 1.5 cases per 100,000 males for severe hemophilia B. Due to the higher mortality rate for people with hemophilia, the estimated prevalence across age groups is lower, with 3.8 cases of hemophilia B per 100,000 males, including 1.1 cases of severe hemophilia per 100,000 males. See, e.g., World Federation of Hemophilia, “Report on the Annual Global Survey 2019” World Federation of Hemophilia (2020), herein incorporated by reference in its entirety for all purposes.

The severity of hemophilia and bleeding manifestations correlates with the degree of clotting factor's activity levels (Table 8). See, e.g., Srivastava et al. (2020) Haemophilia 26.6:1-158, herein incorporated by reference in its entirety for all purposes. In high-income markets, it is estimated that 34% of hemophilia B patients have mild hemophilia, around 31% have a moderate hemophilia, and 33% have a severe hemophilia. See, e.g., World Federation of Hemophilia, “Report on the Annual Global Survey 2019” World Federation of Hemophilia (2020), herein incorporated by reference in its entirety for all purposes.

TABLE 8
Severity Classification of Hemophilia B Based on Clotting Factor
Activity Level.

| Severity | FIX Activity Levels | Clinical Symptoms |
|---|---|---|
| Mild | 6-49% of normal, | Typically experience bleeding only |
| | 0.06-0.40 IU/mL | after serious injury, trauma, or surgery |
| | | May not be diagnosed until well into adulthood. |
| | | Spontaneous bleeding rare, but may occur in patients with less than ~15- |
| | | 30% of normal FIX activity levels. |
| Moderate | 1-5% of normal, | Bleed infrequently, and experience |
| | 0.01-0.05 IU/mL | prolonged bleeding following minor |
| | | surgery or injury. |
| | | Spontaneous bleeds may occur, generally <1 times per month. |
| Severe | <1% of normal, | Experience bleeding after injury and |
| | <0.01 IU/mL | may have frequent spontaneous |
| | | bleeds several times per month, including in their joints and muscles. |

Clinical symptoms of hemophilia B include the observation of easy bruising, “spontaneous” bleeding in the joints, muscles and soft tissues, pain, excessive bleeding following trauma or surgery, and life-threatening intracranial bleeding.

Spontaneous bleeding most commonly occurs in the joints, with the knees (>50% of all bleeding events), elbows, ankles, shoulders, and wrists the most affected. The recurrence of the bleeds in the joints results in inflammation with swelling of the joint, degeneration of the cartilage and progressive destruction of the joint space called hemophilic arthropathy. As the arthropathy develops in a joint, bleedings become more and more frequent even when minimal joint stress is applied such as normal weight bearing, leading to chronic synovitis, pain, fibrosis and progressive joint stiffness. In the last phase of the hemophilic arthropathy, progressive and erosive destruction of the cartilage narrows the joint space leading to collapse or sclerosis of the joint.

Intramuscular hemorrhages represent 30% of bleeding events. When localized in confined spaces like fascial muscles, it may lead to significant compression of vital structures with ischemia, gangrene, contractures and neuropathy. Bleeding in the pelvic space might lead to femoral nerve compression leading to potential permanent disability if neuropathy develops. See, e.g., Napolitano et al., “Chapter 3—Hemophilia A and Hemophilia B,” Consultative Hemostasis and Thrombosis, Elsevier 4(2019):39-58, herein incorporated by reference in its entirety for all purposes.

Epistaxis, oral and gastrointestinal bleeding can also occur following minor trauma like coughing or vomiting. Bleedings in the abdominal wall or in the bowel wall can also occur producing severe pain often misdiagnosed for appendicitis for example. See, e.g., Hoots et al., “Clinical manifestations and diagnosis of hemophilia” UptoDate (2019), herein incorporated by reference in its entirety for all purposes. Hematuria is a frequent manifestation of severe hemophilia but is usually benign and not associated with progressive loss of renal function. See, e.g., Hoots et al., “Clinical manifestations and diagnosis of hemophilia” UptoDate (2019), herein incorporated by reference in its entirety for all purposes.

Intracranial hemorrhage is estimated to occur in approximately 2.7% of individuals with hemophilia and is spontaneous in 50% of the time in affected adults. Despite its low incidence, intracranial hemorrhage is the most common cause of death from bleeding in hemophilic patients. See, e.g., Napolitano et al., “Chapter 3—Hemophilia A and Hemophilia B,” Consultative Hemostasis and Thrombosis, Elsevier 4(2019):39-58, herein incorporated by reference in its entirety for all purposes.

The overall life expectancy of patients with hemophilia B varies whether patients receive appropriate treatment. With prophylaxis and on-demand treatment such as the ones available in the U.S., the median life expectancy was found to be 6 years less than healthy men (77 years old versus 83 years old) in a study conducted in The Netherlands in 2018. See, e.g., Hassan et al. (2021) J. Thromb. Haemost. 19:645-653, herein incorporated by reference in its entirety for all purposes.

Diagnostic. Patients with severe hemophilia B are usually diagnosed before the age of 2 years based on clinical features, whilst patients with mild hemophilia might be diagnosed at an older age if the bleeding symptoms only manifest at time of injuries or surgeries. See, e.g., Berntorp et al. (2021) Nat. Rev. Dis. Primers 7.45, herein incorporated by reference in its entirety for all purposes.

The diagnosis of general hemophilia is firstly based on the clinical features and is then confirmed by screening test results with a normal platelet count, a normal prothrombin time (PT) assay but a prolonged activated partial thromboplastin time (APTT). See, e.g., Srivastava et al. (2020) Haemophilia 26.6:1-158 and Hoots et al., “Clinical manifestations and diagnosis of hemophilia” UptoDate (2019), each of which is herein incorporated by reference in its entirety for all purposes. The final diagnosis of hemophilia B is based on the results of the one-stage FIX assay measuring FIX activity levels below 40% of normal. See, e.g., Srivastava et al. (2020) Haemophilia 26.6:1-158, herein incorporated by reference in its entirety for all purposes. In rare circumstances, the FIX activity level of hemophilia B patients may be ≥40%, and a pathogenic FIX level can be investigated.

A genetic diagnosis can be performed to define the disease biology, establish the diagnosis in difficult cases, predict risk of inhibitor development, identify female carriers, and provide prenatal diagnosis if desired. See, e.g., Srivastava et al. (2020) Haemophilia 26.6:1-158, herein incorporated by reference in its entirety for all purposes. Although it is recognized that genetic testing might not always identify the exact mutation, it is estimated that mutation responsible for hemophilia B is identified in the F9 gene in 98% of cases. See, e.g., Carcao et al., “Hemophilia A and B” Hematology, Elsevier 7(2018):2001-2022, herein incorporated by reference in its entirety for all purposes. Newborns can be tested for hemophilia B through a blood test to measure factor IX levels. For example, the blood can be drawn from the umbilical cord.

Treatment and Unmet Medical Need. The main objective of hemophilia B treatment is to prevent or treat bleedings by replacing the missing blood clotting factor to hemostatically adequate plasma levels for prevention or treatment of acute bleedings. See, e.g., Napolitano et al., “Chapter 3—Hemophilia A and Hemophilia B,” Consultative Hemostasis and Thrombosis, Elsevier 4(2019):39-58, herein incorporated by reference in its entirety for all purposes.

The current standard of care is the use of plasma-derived or recombinant FIX clotting factor concentrates (CFC) for the prevention or treatment of hemophilia B. See, e.g., Srivastava et al. (2020) Haemophilia 26.6:1-158, herein incorporated by reference in its entirety for all purposes. Prophylaxis by regular administration of CFC is recommended to prevent spontaneous bleeding in patients with moderate-to-severe hemophilia B to maintain FIX levels over 1%. FIX activity levels of 30-50% of normal are required to control minor or moderate bleedings or to prevent recurrent spontaneous bleedings (Srivastava et al. (2020) Haemophilia 26.6:1-158), representing 2 to 3 intravenous infusions of standard FIX CFC per week made under the supervision of a physician specialized in hemophilia treatment. See, e.g., Srivastava et al. (2020) Haemophilia 26.6:1-158 and Powell et al. (2013) N. Engl. J. Med. 369.24:2313-2323, each of which is herein incorporated by reference in its entirety for all purposes. Extended half-life CFCs have been developed to reduce the frequency of administration to every week or every two weeks. See, e.g., Powell et al. (2013) N. Engl. J. Med. 369.24:2313-2323, each of which is herein incorporated by reference in its entirety for all purposes. Initiation of prophylaxis is recommended as early as possible, and preferably before 3 years old, since age at initiation has been demonstrated to be a strong predictor of long-term clinical outcomes. See, e.g., Srivastava et al. (2020) Haemophilia 26.6:1-158, herein incorporated by reference in its entirety for all purposes. Gene therapy for hemophilia patients during the neonatal and infant stages can prevent irreversible symptoms and life-threatening events, such as hemophilic arthropathy and intracranial bleeding.

Episodic, or on-demand therapy, can be added to the prophylaxis in case of hemorrhage or surgical procedures in which case levels of 50-100% of normal FIX activity should be achieved and maintained for a minimum of 7 to 10 days. See, e.g., Carcao et al., “Hemophilia A and B” Hematology, Elsevier 7(2018):2001-2022, and Napolitano et al., “Chapter 3—Hemophilia A and Hemophilia B,” Consultative Hemostasis and Thrombosis, Elsevier 4(2019):39-58, each of which is herein incorporated by reference in its entirety for all purposes. On-demand therapy can be the only treatment for patients with moderate forms of hemophilia B who do not experience spontaneous bleeding. See, e.g., Srivastava et al. (2020) Haemophilia 26.6:1-158, herein incorporated by reference in its entirety for all purposes.

Patients with high adherence to prophylaxis treatment have been observed to have lower levels of annualized bleeding rate (ABR). Recent studies on the efficacy of rCFCs observed that the mean ABR across studies ranged between 0-4. See, e.g., Davis et al. (2019) J. Med. Econ. 22.10:1014-1021 and Chhabra (2020) Blood Coagul. Fibrinolysis 31.3:186-192, each of which is herein incorporated by reference in its entirety for all purposes.

In addition to pharmacological treatments, a multi-disciplinary team supporting the patient's care and education relating to hemophilia will be put in place, generally consisting of at least a hematologist, a nurse, and a physical therapist.

Although the use of CFCs in the U.S. has changed the overall course of the disease, there remains an unmet medical need for hemophilia B patients. Firstly, the development of neutralizing antibodies to exogenous FIX administered from the CFCs has been reported to occur in approximately 10% of individuals with severe FIX deficiency which can greatly interfere with the ability to treat bleedings. See, e.g., Male et al. (2021) Hemophilia 106.1:123-129, herein incorporated by reference in its entirety for all purposes. Secondly, prophylaxis treatment requiring several injections per week or an injection every 2 weeks at a dedicated center represents a significant treatment burden on the patient and a notable economic burden to healthcare systems and society. See, e.g., Burke et al. (2021) Orphanet. J. Rare Dis. 16:143, herein incorporated by reference in its entirety for all purposes. Even though prophylaxis drives the risk of spontaneous joint bleeding down, it does not completely eliminate it, leading to remaining chronic pain and disability. See, e.g., Burke et al. (2021) Orphanet. J. Rare Dis. 16:143, herein incorporated by reference in its entirety for all purposes. A treatment that would lead to sustained plasma levels of functional FIX protein on the long-term is thus still needed.

Adeno-associated viral (AAV) vector gene therapies are under investigation for hemophilia B. Clinical trial data demonstrates sustained endogenous production of FIX in some patients which results in the elimination of the need for infusion of replacement FIX. See, e.g., Von Drygalski (2020) Blood 136 (supp 1):13, herein incorporated by reference in its entirety for all purposes. However, AAV expression of FIX is episomal, therefore durability of expression remains unknown until further long-term data on efficacy are generated.

Several recombinant adeno-associated viral (rAAV) vector episomal gene therapies are under Phase 3 investigation for hemophilia B. These rAAV vectors deliver codon-optimized Padua variant human F9 gene associated with a liver-specific promoter to the nuclei of hepatocytes where it will be maintained as an extrachromosomal circular episome. The resulting expressed Padua FIX protein is a variant of the wild type FIX protein which is estimated to have an eight-fold increase in FIX specific activity compared to the wild type FIX. See, e.g., VandenDriessche et al. (2018) Mol. Ther. 26.1:14-16, herein incorporated by reference in its entirety for all purposes. However, AAV expression of FIX is episomal, therefore durability of expression remains unknown until further long-term data on efficacy are generated.

B. Methods

Methods of introducing a F9 nucleic acid into a cell, methods of integration of a F9 nucleic acid into a target genomic locus, methods of expression of FIX in a cell, and methods of treating hemophilia B or FIX deficiency in a subject are provided. Methods of introducing a F9 nucleic acid into a cell or a population of cells (or in a cell or population of cells in a subject), methods of inserting a F9 nucleic acid into a target genomic locus in a cell or a population of cells (or in a cell or population of cells in a subject), methods of expressing a FIX protein in a cell or a population of cells (or in a cell or population of cells in a subject), methods of treating hemophilia B or FIX deficiency in a subject are provided.

The cells or populations of cells can be neonatal cells or populations of neonatal cells, and the subject can be neonatal subjects in some methods. A neonatal subject can be a human subject up to or under the age of 1 year (52 weeks), preferably up to or under the age of 24 weeks, more preferably up to or under the age of 12 weeks, more preferably up to or under the age of 8 weeks, and even more preferably up to or under the age of 4 weeks. In certain embodiments, a neonatal human subject is up to 4 weeks of age. In certain embodiments, a neonatal human subject is up to 8 weeks of age. In another embodiment, a neonatal human subject is within 3 weeks after birth. In another embodiment, a neonatal human subject is within 2 weeks after birth. In another embodiment, a neonatal human subject is within 1 week after birth. In another embodiment, a neonatal human subject is within 7 days after birth. In another embodiment, a neonatal human subject is within 6 days after birth. In another embodiment, a neonatal human subject is within 5 days after birth. In another embodiment, a neonatal human subject is within 4 days after birth. In another embodiment, a neonatal human subject is within 3 days after birth. In another embodiment, a neonatal human subject is within 2 days after birth. In another embodiment, a neonatal human subject is within 1 day after birth. The time windows disclosed above are for human subjects and are also meant to cover the corresponding developmental time windows for other animals. As used herein, a “neonatal cell” is a cell of a neonatal subject, and a population of neonatal cells is a population of cells of a neonatal subject.

The cells or populations of cells can be juvenile cells or populations of juvenile cells, and the subject can be juvenile subjects in some methods. A juvenile subject in the context of humans cover human subjects under the age of 18 years. In one embodiment, a human juvenile subject is a subject within 17 years after birth. In another embodiment, a human juvenile subject is a subject within 16 years after birth. In another embodiment, a human juvenile subject is a subject within 15 years after birth. In another embodiment, a human juvenile subject is a subject within 14 years after birth. In another embodiment, a human juvenile subject is a subject within 13 years after birth. In another embodiment, a human juvenile subject is a subject within 12 years after birth. In another embodiment, a human juvenile subject is a subject within 11 years after birth. In another embodiment, a human juvenile subject is a subject within 10 years after birth. In another embodiment, a human juvenile subject is a subject within 9 years after birth. In another embodiment, a human juvenile subject is a subject within 8 years after birth. In another embodiment, a human juvenile subject is a subject within 7 years after birth. In another embodiment, a human juvenile subject is a subject within 6 years after birth. In another embodiment, a human juvenile subject is a subject within 5 years after birth. In another embodiment, a human juvenile subject is a subject within 4 years after birth. In another embodiment, a human juvenile subject is a subject within 3 years after birth. In another embodiment, a human juvenile subject is a subject within 2 years after birth. In another embodiment, a human juvenile subject is a subject within 1 year after birth. The time windows disclosed above are for human subjects and are also meant to cover the corresponding developmental time windows for other animals. As used herein, a juvenile cell is a cell of a juvenile subject, and a population of juvenile cells is a population of cells of a juvenile subject.

In some methods, the subject has an actively growing liver, or the cells or populations of cells are from an actively growing liver. In other methods, the subject does not have an actively growing liver, or the cells or populations of cells are not from an actively growing liver.

In one example, provided herein are methods of introducing a F9 nucleic acid into a cell or a subject in need thereof. Such methods can comprise administering any of the F9 nucleic acid constructs described herein (or any of the compositions comprising a F9 nucleic acid construct described herein, including, for example, vectors or lipid nanoparticles) to the cell. The F9 nucleic acid construct can be administered together with a nuclease agent described herein, or can be administered alone. In some methods, the F9 nucleic acid construct can be administered together with a nuclease agent described herein. The nuclease agent can cleave a nuclease target sequence within a target gene to create a cleavage site, the F9 nucleic acid construct can be inserted into the cleavage site to create a modified target gene, and FIX protein can be expressed from the modified target gene. The FIX coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target sequence to create a cleavage site, the nucleic acid construct can be inserted into the cleavage site to create a modified ALB gene, and FIX protein can be expressed from the modified ALB gene.

In one example, provided herein are methods of introducing a F9 nucleic acid into a cell or a population of cells or a subject in need thereof (e.g., in a cell or a population of cells in the subject). The cells or populations of cells can be neonatal cells or populations of neonatal cells, and the subject can be neonatal subjects in some methods. In other methods, the cells or populations of cells are not neonatal cells and are not populations of neonatal cells, and the subjects are not neonatal subjects. The cells or populations of cells can be juvenile cells or populations of juvenile cells, and the subject can be juvenile subjects in some methods. In other methods, the cells or populations of cells are not juvenile cells and are not populations of juvenile cells, and the subjects are not juvenile subjects. Such methods can comprise administering any of the F9 nucleic acid constructs described herein (or any of the compositions comprising F9 nucleic acid construct described herein, including, for example, vectors or lipid nanoparticles) to the cell. The F9 nucleic acid construct can be administered together with a nuclease agent described herein, or can be administered alone. In some methods, the F9 nucleic acid construct can be administered together with a nuclease agent described herein (e.g., simultaneously or sequentially in any order). The nuclease agent can cleave a nuclease target sequence within a target genomic locus (e.g., target gene), the F9 nucleic acid construct can be inserted into the target genomic locus to create a modified target genomic locus, and the FIX protein can be expressed from the modified target genomic locus. The F9 coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target sequence, the nucleic acid construct can be inserted into the ALB gene to create a modified ALB gene, and FIX protein can be expressed from the modified ALB gene.

In another example, provided herein are methods of expressing FIX in a cell or a subject in need thereof. Such methods can comprise administering any of the F9 nucleic acid constructs described herein (or any of the compositions comprising a F9 nucleic acid construct described herein, including, for example, vectors or lipid nanoparticles) to the cell. In some methods, the F9 nucleic acid construct or composition comprising the F9 nucleic acid construct can be administered without a nuclease agent (e.g., if the F9 nucleic acid construct comprises elements needed for expression of FIX without integration into a target genomic locus). In some methods, the F9 nucleic acid construct can be administered together with a nuclease agent described herein. The nuclease agent can cleave a nuclease target sequence within a target gene to create a cleavage site, the F9 nucleic acid construct can be inserted into the cleavage site to create a modified target gene, and FIX protein can be expressed from the modified target gene. The FIX coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target sequence to create a cleavage site, the nucleic acid construct can be inserted into the cleavage site to create a modified ALB gene, and FIX protein can be expressed from the modified ALB gene.

In another example, provided herein are methods of expressing a FIX protein in a cell or a population of cells or a subject in need thereof (e.g., in a cell or a population of cells in the subject). The cells or populations of cells can be neonatal cells or populations of neonatal cells, and the subject can be neonatal subjects in some methods. In other methods, the cells or populations of cells are not neonatal cells and are not populations of neonatal cells, and the subjects are not neonatal subjects. The cells or populations of cells can be juvenile cells or populations of juvenile cells, and the subject can be juvenile subjects in some methods. In other methods, the cells or populations of cells are not juvenile cells and are not populations of juvenile cells, and the subjects are not juvenile subjects. Such methods can comprise administering any of the F9 nucleic acid constructs described herein (or any of the compositions comprising a F9 nucleic acid construct described herein, including, for example, vectors or lipid nanoparticles) to the cell. In some methods, the F9 nucleic acid construct or composition comprising the F9 nucleic acid construct can be administered without a nuclease agent (e.g., if the F9 nucleic acid construct comprises elements needed for expression of the FIX protein without integration into a target genomic locus). In some methods, the F9 nucleic acid construct can be administered together with a nuclease agent described herein (e.g., simultaneously or sequentially in any order). The nuclease agent can cleave a nuclease target sequence within a target genomic locus (e.g., target gene), the F9 nucleic acid construct can be inserted into the target genomic locus to create a modified target genomic locus, and FIX protein can be expressed from the modified target genomic locus. The F9 coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target sequence, the nucleic acid construct can be inserted into the ALB gene to create a modified ALB gene, and FIX protein can be expressed from the modified ALB gene.

In another example, provided herein are methods of integrating a F9 nucleic acid construct into a target genomic locus in a cell or a subject in need thereof. Such methods can comprise administering any of the F9 nucleic acid constructs described herein (or any of the compositions comprising a F9 nucleic acid construct described herein, including, for example, vectors or lipid nanoparticles) to the cell. In some methods, the F9 nucleic acid construct or composition comprising the F9 nucleic acid construct can be administered together with a nuclease agent described herein. The nuclease agent can cleave a nuclease target sequence within a target gene to create a cleavage site, the F9 nucleic acid construct can be inserted into the cleavage site to create a modified target gene, and FIX protein can be expressed from the modified target gene. The FIX coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target sequence to create a cleavage site, the nucleic acid construct can be inserted into the cleavage site to create a modified ALB gene, and FIX protein can be expressed from the modified ALB gene.

In another example, provided herein are methods of inserting or integrating a F9 nucleic acid construct into a target genomic locus in a cell or a population of cells or a subject in need thereof (e.g., in a cell or a population of cells in the subject). The cells or populations of cells can be neonatal cells or populations of neonatal cells, and the subject can be neonatal subjects in some methods. In other methods, the cells or populations of cells are not neonatal cells and are not populations of neonatal cells, and the subjects are not neonatal subjects. The cells or populations of cells can be juvenile cells or populations of juvenile cells, and the subject can be juvenile subjects in some methods. In other methods, the cells or populations of cells are not juvenile cells and are not populations of juvenile cells, and the subjects are not juvenile subjects. Such methods can comprise administering any of the F9 nucleic acid constructs described herein (or any of the compositions comprising a F9 nucleic acid construct described herein, including, for example, vectors or lipid nanoparticles) to the cell. In some methods, the F9 nucleic acid construct or composition comprising the F9 nucleic acid construct can be administered together with a nuclease agent described herein (e.g., simultaneously or sequentially in any order). The nuclease agent can cleave a nuclease target sequence within a target genomic locus (e.g., target gene), the F9 nucleic acid construct can be inserted into the target genomic locus to create a modified target genomic locus, and the FIX protein can be expressed from the modified target genomic locus. The F9 coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target sequence, the nucleic acid construct can be inserted into the ALB gene to create a modified ALB gene, and FIX protein can be expressed from the modified ALB gene.

In any of the above methods, the cells can be from any suitable species, such as eukaryotic cells or mammalian cells (e.g., non-human mammalian cells or human cells). A mammal can be, for example, a non-human mammal, a human, a rodent, a rat, a mouse, or a hamster. Other non-human mammals include, for example, non-human primates, monkeys, apes, cats, dogs, rabbits, horses, bulls, deer, bison, livestock (e.g., bovine species such as cows, steer, and so forth; ovine species such as sheep, goats, and so forth; and porcine species such as pigs and boars). Birds include, for example, chickens, turkeys, ostrich, geese, ducks, and so forth. Domesticated animals and agricultural animals are also included. The term “non-human” excludes humans. Specific examples include, but are not limited to, human cells, rodent cells, mouse cells, rat cells, and non-human primate cells. In a specific example, the cell is a human cell. Likewise, cells can be any suitable type of cell. In a specific example, the cell is a liver cell such as a hepatocyte (e.g., a human liver cell or human hepatocyte).

The cells can be isolated cells (e.g., in vitro), ex vivo cells, or can be in vivo within an animal (i.e., in a subject). In a specific example, the cell is in vivo (e.g., in a subject having a FIX deficiency or hemophilia (e.g., hemophilia B)). The cells can be mitotically competent cells or mitotically-inactive cells, meiotically competent cells or meiotically-inactive cells. Similarly, the cells can also be primary somatic cells or cells that are not a primary somatic cell. Somatic cells include any cell that is not a gamete, germ cell, gametocyte, or undifferentiated stem cell. For example, the cells can be liver cells, such as hepatocytes (e.g., mouse, non-human primate, or human hepatocytes).

The cells provided herein can be normal, healthy cells, or can be diseased or mutant-bearing cells. For example, the cells can have a FIX deficiency or can be from a subject with FIX deficiency or hemophilia (e.g., hemophilia B).

The cells can be dividing cells (e.g., actively dividing cells). The cells can also be non-dividing cells. A non-dividing cell refers to cells that are terminally differentiated and do not divide, as well as quiescent cells that do not divide but retains the ability to re-enter cell division and proliferation. Liver cells, for example, retain the ability to divide (e.g., when injured or resected), but do not typically divide. During mitotic cell division, homologous recombination is a mechanism by which the genome is protected and double-stranded breaks are repaired. A non-dividing cell can refer to a cell in which homologous recombination (HR) is not the primary mechanism by which double-stranded DNA breaks are repaired in the cell (e.g., as compared to a control dividing cell). A non-dividing cell can refer to a cell in which non-homologous end joining (NHEJ) is the primary mechanism by which double-stranded DNA breaks are repaired in the cell (e.g., as compared to a control dividing cell). Non-dividing cell types have been described in the literature, for example, by active NHEJ double-stranded DNA break repair mechanisms. See, e.g. Iyama, DNA Repair (Amst.) 2013, 12(8): 620-636

Also provided are methods of treating a FIX deficiency in a subject and methods of treating hemophilia B in a subject and methods of preventing or inhibiting spontaneous bleeding in a subject having hemophilia B. The hemophilia B can be any type of hemophilia B (e.g., mild hemophilia B, moderate hemophilia B, or severe hemophilia B). Hemophilia B is described in more detail elsewhere herein. Such methods can comprise administering any of the F9 nucleic acid constructs described herein (or any of the compositions comprising a F9 nucleic acid construct described herein, including, for example, vectors or lipid nanoparticles) to the subject such that a therapeutically effective level of FIX expression is achieved in the subject. In some methods, the F9 nucleic acid construct or composition comprising the F9 nucleic acid construct can be administered without a nuclease agent (e.g., if the F9 nucleic acid construct comprises elements needed for expression of FIX without integration into a target genomic locus). In some methods, the F9 nucleic acid construct can be administered together with a nuclease agent described herein. The nuclease agent can cleave a nuclease target sequence within a target gene to create a cleavage site, the F9 nucleic acid construct can be inserted into the cleavage site to create a modified target gene, and FIX protein can be expressed from the modified target gene such that a therapeutically effective level of FIX expression is achieved in the subject. The FIX coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target sequence to create a cleavage site, the nucleic acid construct can be inserted into the cleavage site to create a modified ALB gene, and FIX protein can be expressed from the modified ALB gene such that a therapeutically effective level of FIX expression is achieved in the subject.

Also provided are methods of treating a FIX deficiency in a subject and methods of treating hemophilia B in a subject and methods of preventing or inhibiting spontaneous bleeding in a subject having hemophilia B. The subject can be a neonatal subject in some methods. In other methods, the subjects are not neonatal subjects. The subject can be a juvenile subjects in some methods. In other methods, the subjects are not juvenile subjects. The hemophilia B can be any type of hemophilia B (e.g., mild hemophilia B, moderate hemophilia B, or severe hemophilia B). Hemophilia B is described in more detail elsewhere herein. Such methods can comprise administering any of the F9 nucleic acid constructs described herein (or any of the compositions comprising a F9 nucleic acid construct described herein, including, for example, vectors or lipid nanoparticles) to the subject such that a therapeutically effective level of FIX protein expression or a therapeutically effective level of circulating FIX protein is achieved in the subject. In some methods, the F9 nucleic acid construct or composition comprising the F9 nucleic acid construct can be administered without a nuclease agent (e.g., if the F9 nucleic acid construct comprises elements needed for expression of FIX protein without integration into a target genomic locus). In some methods, the F9 nucleic acid construct can be administered together with a nuclease agent described herein (e.g., simultaneously or sequentially in any order). The nuclease agent can cleave a nuclease target sequence within a target genomic locus (e.g., target gene), the F9 nucleic acid construct can be inserted into the target genomic locus to create a modified target genomic locus, and the FIX protein can be expressed from the modified target genomic locus (e.g., such that a therapeutically effective level of FIX protein expression or a therapeutically effective level of circulating FIX protein is achieved in the subject). The F9 coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid construct can be inserted into the ALB gene to create a modified ALB gene, and FIX protein can be expressed from the modified ALB gene (e.g., such that a therapeutically effective level of FIX protein expression or a therapeutically effective level of circulating FIX protein is achieved in the subject).

Treatment refers to any administration or application of a therapeutic for disease or disorder in a subject, and includes inhibiting the disease, arresting its development, relieving one or more symptoms of the disease, curing the disease, or preventing reoccurrence of one or more symptoms of the disease. For example, treatment of hemophilia B may comprise alleviating symptoms of hemophilia B. In one specific example, a method of preventing or inhibiting spontaneous bleeding in a subject having hemophilia B is provided. Hemophilia B is described in detail above and refers to a disorder caused by a missing or defective F9 gene or FIX polypeptide. The disorder includes conditions that are inherited and/or acquired (e.g., caused by a spontaneous mutation in the gene). The defective F9 gene or FIX polypeptide can result in reduced FIX level in the plasma and/or a reduced coagulation activity of FIX. Hemophilia B includes mild, moderate, and severe hemophilia B. For example, individuals with less than about 1% active factor are classified as having severe hemophilia, those with about 1-5% active factor have moderate hemophilia, and those with mild hemophilia have between about 5-40% of normal levels of active clotting factor. As used herein, “normal” or “healthy” individuals include those having between 50 and 160% of normal pooled plasma level of FIX activity and antigen levels. In one example, normal plasma FIX levels are about 3-5 μg/mL. In a specific example, normal FIX activity is considered to be about 100% of normal pooled plasma level of FIX activity or is considered to be 100% of normal pooled plasma level of FIX activity. In a specific example, normal plasma FIX levels are considered to be about 5 μg/mL or are considered to be 5 μg/mL. In some embodiments, the level of FIX (e.g., circulating FIX) can be measured by a coagulation and/or an immunologic assay. FIX procoagulant activity can be determined by the ability of the patient's plasma to correct the clotting time of FIX-deficient plasma.

In some methods, a therapeutically effective amount of the F9 nucleic acid construct or the composition comprising the F9 nucleic acid construct or the combination of the F9 nucleic acid construct and the nuclease agent (e.g., CRISPR/Cas system) is administered to the subject. A therapeutically effective amount is an amount that produces the desired effect for which it is administered. The exact amount will depend on the purpose of the treatment, and will be ascertainable by one skilled in the art using known techniques. See, e.g., Lloyd (1999) The Art, Science and Technology of Pharmaceutical Compounding.

Therapeutic or pharmaceutical compositions comprising the compositions disclosed herein can be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, Pa. See also Powell et al. “Compendium of excipients for parenteral formulations” PDA (1998) J. Pharm. Sci. Technol. 52:238-311.

The compositions disclosed herein may be administered to relieve or prevent or decrease the severity of one or more of the symptoms of FIX deficiency or hemophilia B. Such symptoms are described in more detail elsewhere herein.

The subject in any of the above methods can be one in need of amelioration or treatment of FIX deficiency or hemophilia B. The subject in any of the above methods can be from any suitable species, such as a eukaryote or a mammal. A mammal can be, for example, a non-human mammal, a human, a rodent, a rat, a mouse, or a hamster. Other non-human mammals include, for example, non-human primates, monkeys, apes, cats, dogs, rabbits, horses, bulls, deer, bison, livestock (e.g., bovine species such as cows, steer, and so forth; ovine species such as sheep, goats, and so forth; and porcine species such as pigs and boars). Birds include, for example, chickens, turkeys, ostrich, geese, ducks, and so forth. Domesticated animals and agricultural animals are also included. The term “non-human” excludes humans. Specific examples of suitable species include, but are not limited to, humans, rodents, mice, rats, and non-human primates. In a specific example, the subject is a human.

In methods in which a F9 nucleic acid construct is genomically integrated, any target genomic locus capable of expressing a gene can be used, such as a safe harbor locus (safe harbor gene) or an endogenous F9 locus. Such loci are described in more detail elsewhere herein. In a specific example, the target genomic locus can be an endogenous ALB locus, such as an endogenous human ALB locus. For example, the nucleic acid construct can be genomically integrated in intron 1 of the endogenous ALB locus. Endogenous ALB exon 1 can then splice into the coding sequence for the FIX protein in the nucleic acid construct.

Targeted insertion of the F9 nucleic acid construct comprising the FIX coding sequence into a target genomic locus, and particularly an endogenous ALB locus, offers multiple advantages. Such methods result in stable modification to allow for stable, long-term expression of the FIX coding sequence. With respect to the ALB locus, such methods are able to utilize the endogenous ALB promoter and regulatory regions to achieve therapeutically effective levels of expression. For example, the FIX coding sequence in the nucleic acid construct can comprise a promoterless gene, and the inserted nucleic acid construct can be operably linked to an endogenous promoter in the target genomic locus (e.g., ALB locus). Use of an endogenous promoter is advantageous because it obviates the need for inclusion of a promoter in the nucleic acid construct, allowing packaging of larger transgenes that may not normally package efficiently (e.g., in AAV). Alternatively, the FIX coding sequence in the nucleic acid construct can be operably linked to an exogenous promoter in the nucleic acid construct. Examples of types of promoters that can be used are disclosed elsewhere herein.

Optionally, some or all of the endogenous gene (e.g., endogenous ALB gene) at the target genomic locus can be expressed upon insertion of the FIX coding sequence from the nucleic acid construct. Alternatively, in some methods, none of the endogenous gene at the target genomic locus is expressed. As one example, the modified target genomic locus (e.g., modified ALB locus) after integration of the nucleic acid construct can encode a chimeric protein comprising an endogenous secretion signal (e.g., albumin secretion signal) and the FIX protein encoded by the nucleic acid construct. In another example, the first intron of an ALB locus can be targeted. The secretion signal peptide of ALB is encoded by exon 1 of the ALB gene. In such a scenario, a promoterless cassette bearing a splice acceptor and the FIX coding sequence will support expression and secretion of the FIX protein. Splicing between endogenous ALB exon 1 and the integrated FIX coding sequence creates a chimeric mRNA and protein including the endogenous ALB sequence encoded by exon 1 operably linked to the FIX protein sequence encoded by the integrated nucleic acid construct.

The F9 nucleic acid construct can be inserted into the target genomic locus by any means, including homologous recombination (HR) and non-homologous end joining (NHEJ) as described elsewhere herein. In a specific example, the F9 nucleic acid construct is inserted by NHEJ (e.g., does not comprise homology arms and is inserted by NHEJ).

In another specific example, the nucleic acid construct can be inserted via homology-independent targeted integration (e.g., directional homology-independent targeted integration). For example, the FIX coding sequence in the nucleic acid construct can be flanked on each side by a target site for a nuclease agent (e.g., the same target site as in the target genomic locus, and the same nuclease agent being used to cleave the target site in the target genomic locus). The nuclease agent can then cleave the target sites flanking the FIX coding sequence. In a specific example, the nucleic acid construct is delivered AAV-mediated delivery, and cleavage of the target sites flanking the FIX coding sequence can remove the inverted terminal repeats (ITRs) of the AAV. Removal of the ITRs can make it easier to assess successful targeting, because presence of the ITRs can hamper sequencing efforts due to the repeated sequences. In some methods, the target site in the target genomic locus (e.g., a gRNA target sequence including the flanking protospacer adjacent motif) is no longer present if the FIX coding sequence is inserted into the target genomic locus in the correct orientation but it is reformed if the FIX protein coding sequence is inserted into the target genomic locus in the opposite orientation. This can help ensure that the FIX coding sequence is inserted in the correct orientation for expression.

In any of the above methods, the F9 nucleic acid construct can be administered simultaneously with the nuclease agent (e.g., CRISPR/Cas system) or not simultaneously (e.g., sequentially in any combination). For example, in a method comprising administering a composition comprising the F9 nucleic acid construct and a nuclease agent, they can be administered separately. For example, the F9 nucleic acid construct can be administered prior to the nuclease agent, subsequent to the nuclease agent, or at the same time as the nuclease agent. Any suitable methods of administering nucleic acid constructs and nuclease agents to cells can be used, particularly methods of administering to the liver, and examples of such methods are described in more detail elsewhere herein. In methods of treatment or in methods of targeting a cell in vivo in a subject, the nucleic acid construct can be inserted in particular types of cells in the subject. The method and vehicle for introducing the F9 nucleic acid construct and/or the nuclease agent into the subject can affect which types of cells in the subject are targeted. In some methods, for example, the nucleic acid construct is inserted into a target genomic locus (e.g., an endogenous ALB locus) in liver cells, such as hepatocytes. Methods and vehicles for introducing such constructs and nuclease agents into the subject (including methods and vehicles that target the liver or hepatocytes, such as lipid nanoparticle-mediated delivery and AAV-mediated delivery (e.g., rAAV8-mediated delivery) and intravenous injection), are disclosed in more detail elsewhere herein.

In methods in which a compositions comprising a nucleic acid construct (or vector or LNP) and a nuclease agent is administered (i.e., in methods in which a nucleic acid construct (or vector or LNP) and a nuclease agent are both administered), the nucleic acid construct and the nuclease agent can be administered simultaneously. Alternatively, the nucleic acid construct and the nuclease agent can be administered sequentially in any order. For example, the nucleic acid construct can be administered after the nuclease agent, or the nuclease agent can be administered after the nucleic acid construct. For example, the nuclease agent can be administered about 1 hour to about 48 hours, about 1 hour to about 24 hours, about 1 hour to about 12 hours, about 1 hour to about 6 hours, about 1 hour to about 2 hours, about 2 hours to about 48 hours, about 2 hours to about 24 hours, about 2 hours to about 12 hours, about 2 hours to about 6 hours, about 3 hours to about 48 hours, about 6 hours to about 48 hours, about 12 hours to about 48 hours, or about 24 hours to about 48 hours prior to or subsequent to administration of the nucleic acid construct.

In one example, the nucleic acid construct is administered about 4 hours, about 8 hours, about 12 hours, about 18 hours, about 1 day, about 2 days, about 3 days, about 4 days, about 5 days, about 6 days, or about 1 week prior to administering the nuclease agent. In another example, the nucleic acid construct is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week prior to administering the nuclease agent. In another example, the nucleic acid construct is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days prior to administering the nuclease agent.

In one example, the nucleic acid construct is administered about 4 hours, about 8 hours, about 12 hours, about 18 hours, about 1 day, about 2 days, about 3 days, about 4 days, about 5 days, about 6 days, or about 1 week after administering the nuclease agent. In another example, the nucleic acid construct is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week after administering the nuclease agent. In another example, the nucleic acid construct is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days after administering the nuclease agent.

In any of the above methods, the F9 nucleic acid construct and the nuclease agent (e.g., CRISPR/Cas system) can be administered using any suitable delivery system and known method. The nuclease agent components and F9 nucleic acid construct (e.g., the guide RNA, Cas protein, and F9 nucleic acid construct) can be delivered individually or together in any combination, using the same or different delivery methods as appropriate.

In methods in which a CRISPR/Cas system is used, a guide RNA can be introduced into or administered to a subject or cell, for example, in the form of an RNA (e.g., in vitro transcribed RNA, such as the modified guide RNAs disclosed herein) or in the form of a DNA encoding the guide RNA. When introduced in the form of a DNA, the DNA encoding a guide RNA can be operably linked to a promoter active in the cell or in a cell in the subject. For example, a guide RNA may be delivered via AAV and expressed in vivo under a U6 promoter. Such DNAs can be in one or more expression constructs. For example, such expression constructs can be components of a single nucleic acid molecule. Alternatively, they can be separated in any combination among two or more nucleic acid molecules (i.e., DNAs encoding one or more CRISPR RNAs and DNAs encoding one or more tracrRNAs can be components of a separate nucleic acid molecules).

Likewise, Cas proteins can be introduced into a subject or cell in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)), such as a modified mRNA as disclosed herein, or DNA). Optionally, the nucleic acid encoding the Cas protein can be codon optimized for efficient translation into protein in a particular cell or organism. For example, the nucleic acid encoding the Cas protein can be modified to substitute codons having a higher frequency of usage in a mammalian cell, a human cell, a rodent cell, a mouse cell, a rat cell, or any other host cell of interest, as compared to the naturally occurring polynucleotide sequence. When a nucleic acid encoding the Cas protein is introduced into a cell or a subject, the Cas protein can be transiently, conditionally, or constitutively expressed in the cell or in a cell in the subject.

In one example, the Cas protein is introduced in the form of an mRNA (e.g., a modified mRNA as disclosed herein), and the guide RNA is introduced in the form of RNA such as a modified gRNA as disclosed herein (e.g., together within the same lipid nanoparticle). Guide RNAs can be modified as disclosed elsewhere herein. Likewise, Cas mRNAs can be modified as disclosed elsewhere herein.

In methods in which a F9 nucleic acid construct is inserted following cleavage by a gene-editing system (e.g., a Cas protein), the gene-editing system (e.g., Cas protein) can cleave the target genomic locus to create a single-strand break (nick) or double-strand break, and the cleaved or nicked locus can be repaired by insertion of the F9 nucleic acid construct via non-homologous end joining (NHEJ)-mediated insertion or homology-directed repair. Optionally, repair with the F9 nucleic acid construct removes or disrupts the guide RNA target sequence(s) so that alleles that have been targeted cannot be re-targeted by the CRISPR/Cas reagents.

As explained in more detail elsewhere herein, the F9 nucleic acid constructs can comprise deoxyribonucleic acid (DNA) or ribonucleic acid (RNA), they can be single-stranded or double-stranded, and they can be in linear or circular form. The F9 nucleic acid constructs can be naked nucleic acids or can be delivered by viruses, such as AAV. In a specific example, the F9 nucleic acid construct can be delivered via AAV and can be capable of insertion into the target genomic locus (e.g., a safe harbor gene, an ALB gene, or intron 1 of an ALB gene) by non-homologous end joining (e.g., the F9 nucleic acid construct can be one that does not comprise homology arms).

Some F9 nucleic acid constructs are capable of insertion by non-homologous end joining. In some cases, such F9 nucleic acid constructs do not comprise homology arms. For example, such F9 nucleic acid constructs can be inserted into a blunt end double-strand break following cleavage with a Cas protein. In a specific example, the F9 nucleic acid construct can be delivered via AAV and can be capable of insertion by non-homologous end joining (e.g., the F9 nucleic acid construct can be one that does not comprise homology arms).

In another example, the F9 nucleic acid construct can be inserted via homology-independent targeted integration. For example, the F9 nucleic acid construct can be flanked on each side by a guide RNA target sequence (e.g., the same target site as in the target genomic locus, and the CRISPR/Cas reagent (Cas protein and guide RNA) being used to cleave the target site in the target genomic locus). The Cas protein can then cleave the target sites flanking the nucleic acid insert. In a specific example, the F9 nucleic acid construct is delivered AAV-mediated delivery, and cleavage of the target sites flanking the nucleic acid insert can remove the inverted terminal repeats (ITRs) of the AAV. In some methods, the target site in the target genomic locus (e.g., a guide RNA target sequence including the flanking protospacer adjacent motif) is no longer present if the nucleic acid insert is inserted into the target genomic locus in the correct orientation but it is reformed if the nucleic acid insert is inserted into the target genomic locus in the opposite orientation.

The methods disclosed herein can comprise introducing or administering into an subject (e.g., an animal or mammal, such as a human) or cell a F9 nucleic acid construct and optionally a nuclease agent such as CRISPR/Cas reagents, including in the form of nucleic acids (e.g., DNA or RNA), proteins, or nucleic-acid-protein complexes. “Introducing” or “administering” includes presenting to the cell or subject the molecule(s) (e.g., nucleic acid(s) or protein(s)) in such a manner that it gains access to the interior of the cell or to the interior of cells within the subject. The introducing can be accomplished by any means, and two or more of the components (e.g., two of the components, or all of the components) can be introduced into the cell or subject simultaneously or sequentially in any combination. For example, a Cas protein can be introduced into a cell or subject before introduction of a guide RNA, or it can be introduced following introduction of the guide RNA. As another example, a F9 nucleic acid construct can be introduced prior to the introduction of a Cas protein and a guide RNA, or it can be introduced following introduction of the Cas protein and the guide RNA (e.g., the F9 nucleic acid construct can be administered about 1, 2, 3, 4, 8, 12, 24, 36, 48, or 72 hours before or after introduction of the Cas protein and the guide RNA). See, e.g., US 2015/0240263 and US 2015/0110762, each of which is herein incorporated by reference in its entirety for all purposes. In addition, two or more of the components can be introduced into the cell or subject by the same delivery method or different delivery methods. Similarly, two or more of the components can be introduced into a subject by the same route of administration or different routes of administration.

A guide RNA can be introduced into a subject or cell, for example, in the form of an RNA (e.g., in vitro transcribed RNA) or in the form of a DNA encoding the guide RNA. Guide RNAs can be modified as disclosed elsewhere herein. When introduced in the form of a DNA, the DNA encoding a guide RNA can be operably linked to a promoter active in the cell or in a cell in the subject. For example, a guide RNA may be delivered via AAV and expressed in vivo under a U6 promoter. Such DNAs can be in one or more expression constructs. For example, such expression constructs can be components of a single nucleic acid molecule. Alternatively, they can be separated in any combination among two or more nucleic acid molecules (i.e., DNAs encoding one or more CRISPR RNAs and DNAs encoding one or more tracrRNAs can be components of a separate nucleic acid molecules).

Likewise, Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Cas RNAs can be modified as disclosed elsewhere herein. Optionally, the nucleic acid encoding the Cas protein can be codon optimized for efficient translation into protein in a particular cell or organism. For example, the nucleic acid encoding the Cas protein can be modified to substitute codons having a higher frequency of usage in a mammalian cell, a human cell, a rodent cell, a mouse cell, a rat cell, or any other host cell of interest, as compared to the naturally occurring polynucleotide sequence. When a nucleic acid encoding the Cas protein is introduced into a cell or a subject, the Cas protein can be transiently, conditionally, or constitutively expressed in the cell or in a cell in the subject.

Nucleic acids encoding Cas proteins or guide RNAs can be operably linked to a promoter in an expression construct. Expression constructs include any nucleic acid constructs capable of directing expression of a gene or other nucleic acid sequence of interest (e.g., a Cas gene) and which can transfer such a nucleic acid sequence of interest to a target cell. For example, the nucleic acid encoding the Cas protein can be in a vector comprising a DNA encoding one or more gRNAs. Alternatively, it can be in a vector or plasmid that is separate from the vector comprising the DNA encoding one or more gRNAs. Suitable promoters that can be used in an expression construct include promoters active, for example, in one or more of a eukaryotic cell, a human cell, a non-human cell, a mammalian cell, a non-human mammalian cell, a rodent cell, a mouse cell, a rat cell, a hamster cell, a rabbit cell, a pluripotent cell, an embryonic stem (ES) cell, an adult stem cell, a developmentally restricted progenitor cell, an induced pluripotent stem (iPS) cell, or a one-cell stage embryo. For example, a suitable promoter can be active in a liver cell such as a hepatocyte. Such promoters can be, for example, conditional promoters, inducible promoters, constitutive promoters, or tissue-specific promoters. Optionally, the promoter can be a bidirectional promoter driving expression of both a Cas protein in one direction and a guide RNA in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express genes encoding a Cas protein and a guide RNA simultaneously allows for the generation of compact expression cassettes to facilitate delivery.

Molecules (e.g., Cas proteins or guide RNAs or nucleic acids encoding) introduced into the subject or cell can be provided in compositions comprising a carrier increasing the stability of the introduced molecules (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules.

Various methods and compositions are provided herein to allow for introduction of molecule (e.g., a nucleic acid or protein) into a cell or subject. Methods for introducing molecules into various cell types are known and include, for example, stable transfection methods, transient transfection methods, and virus-mediated methods.

Transfection protocols as well as protocols for introducing molecules into cells may vary. Non-limiting transfection methods include chemical-based transfection methods using liposomes; nanoparticles; calcium phosphate (Graham et al. (1973) Virology 52 (2): 456-67, Bacchetti et al. (1977) Proc. Natl. Acad. Sci. U.S.A. 74 (4):1590-4, and Kriegler, M (1991). Transfer and Expression: A Laboratory Manual. New York: W. H. Freeman and Company. pp. 96-97); dendrimers; or cationic polymers such as DEAE-dextran or polyethylenimine. Non-chemical methods include electroporation, sonoporation, and optical transfection. Particle-based transfection includes the use of a gene gun, or magnet-assisted transfection (Bertram (2006) Current Pharmaceutical Biotechnology 7, 277-28). Viral methods can also be used for transfection.

Introduction of nucleic acids or proteins into a cell can also be mediated by electroporation, by intracytoplasmic injection, by viral infection, by adenovirus, by adeno-associated virus, by lentivirus, by retrovirus, by transfection, by lipid-mediated transfection, or by nucleofection. Nucleofection is an improved electroporation technology that enables nucleic acid substrates to be delivered not only to the cytoplasm but also through the nuclear membrane and into the nucleus. In addition, use of nucleofection in the methods disclosed herein typically requires much fewer cells than regular electroporation (e.g., only about 2 million compared with 7 million by regular electroporation). In one example, nucleofection is performed using the LONZA® NUCLEOFECTOR™ system.

Introduction of molecules (e.g., nucleic acids or proteins) into a cell (e.g., a zygote) can also be accomplished by microinjection. In zygotes (i.e., one-cell stage embryos), microinjection can be into the maternal and/or paternal pronucleus or into the cytoplasm. If the microinjection is into only one pronucleus, the paternal pronucleus is preferable due to its larger size. Microinjection of an mRNA is preferably into the cytoplasm (e.g., to deliver mRNA directly to the translation machinery), while microinjection of a Cas protein or a polynucleotide encoding a Cas protein or encoding an RNA is preferable into the nucleus/pronucleus. Alternatively, microinjection can be carried out by injection into both the nucleus/pronucleus and the cytoplasm: a needle can first be introduced into the nucleus/pronucleus and a first amount can be injected, and while removing the needle from the one-cell stage embryo a second amount can be injected into the cytoplasm. If a Cas protein is injected into the cytoplasm, the Cas protein preferably comprises a nuclear localization signal to ensure delivery to the nucleus/pronucleus. Methods for carrying out microinjection are well known. See, e.g., Nagy et al. (Nagy A, Gertsenstein M, Vintersten K, Behringer R., 2003, Manipulating the Mouse Embryo. Cold Spring Harbor, New York: Cold Spring Harbor Laboratory Press); see also Meyer et al. (2010) Proc. Natl. Acad. Sci. U.S.A. 107:15022-15026 and Meyer et al. (2012) Proc. Natl. Acad. Sci. U.S.A. 109:9354-9359, each of which is herein incorporated by reference in its entirety for all purposes.

Other methods for introducing molecules (e.g., nucleic acid or proteins) into a cell or subject can include, for example, vector delivery, particle-mediated delivery, exosome-mediated delivery, lipid-nanoparticle-mediated delivery, cell-penetrating-peptide-mediated delivery, or implantable-device-mediated delivery. As specific examples, a nucleic acid or protein can be introduced into a cell or subject in a carrier such as a poly(lactic acid) (PLA) microsphere, a poly(D,L-lactic-coglycolic-acid) (PLGA) microsphere, a liposome, a micelle, an inverse micelle, a lipid cochleate, or a lipid microtubule. Some specific examples of delivery to a subject include hydrodynamic delivery, virus-mediated delivery (e.g., adeno-associated virus (AAV)-mediated delivery), and lipid-nanoparticle-mediated delivery.

Introduction of nucleic acids and proteins into cells or subjects can be accomplished by hydrodynamic delivery (HDD). For gene delivery to parenchymal cells, only essential DNA sequences need to be injected via a selected blood vessel, eliminating safety concerns associated with current viral and synthetic vectors. When injected into the bloodstream, DNA is capable of reaching cells in the different tissues accessible to the blood. Hydrodynamic delivery employs the force generated by the rapid injection of a large volume of solution into the incompressible blood in the circulation to overcome the physical barriers of endothelium and cell membranes that prevent large and membrane-impermeable compounds from entering parenchymal cells. In addition to the delivery of DNA, this method is useful for the efficient intracellular delivery of RNA, proteins, and other small compounds in vivo. See, e.g., Bonamassa et al. (2011) Pharm. Res. 28(4):694-701, herein incorporated by reference in its entirety for all purposes.

Introduction of nucleic acids can also be accomplished by virus-mediated delivery, such as AAV-mediated delivery or lentivirus-mediated delivery. Other exemplary viruses/viral vectors include retroviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression, long-lasting expression (e.g., at least 1 week, 2 weeks, 1 month, 2 months, or 3 months), or permanent expression (e.g., of Cas9 and/or gRNA). Viral vector may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper virus to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include about 10¹², about 10¹³, about 10¹⁴, about 10¹⁵, and about 10¹⁶ vector genomes (vg)/mL, or between about 10¹² to about 10¹⁶, between about 10¹² to about 10¹⁵, between about 10¹² to about 10¹⁴, between about 10¹² to about 10¹³, between about 10¹³ to about 10¹⁶, between about 10¹⁴ to about 10¹⁶, between about 10¹⁵ to about 10¹⁶, or between about 10¹³ to about 10¹⁵ vg/mL. Other exemplary viral titers (e.g., AAV titers) include about 10¹², about 10¹³, about 10¹⁴, about 10¹⁵, and about 10¹⁶ vector genomes (vg)/kg of body weight, or between about 10¹² to about 10¹⁶, between about 10¹² to about 10¹⁵, between about 10¹² to about 10¹⁴, between about 10¹² to about 10¹³, between about 10¹³ to about 10¹⁶, between about 10¹⁴ to about 10¹⁶, between about 10¹⁵ to about 10¹⁶, or between about 10¹³ to about 10¹⁵ vg/kg of body weight. In one example, the viral titer is between about 10¹³ to about 10¹⁴ vg/mL or vg/kg. In another example, the viral titer is between about 10¹² to about 10¹³ vg/mL or vg/kg (e.g., between about 10¹² to about 10¹³ vg/kg). In another example, the viral titer is between about 10¹² to about 10¹⁴ vg/mL or vg/kg (e.g., between about 10¹² to about 10¹⁴ vg/kg). For example, the viral titer can be between about 1.5E12 to about 1.5E13 vg/kg, can be about 1.5E12 vg/kg, or can be about 1.5E13 vg/kg. AAVs for use in the methods are discussed in more detail elsewhere herein.

Introduction of nucleic acids and proteins can also be accomplished by lipid nanoparticle (LNP)-mediated delivery. For example, LNP-mediated delivery can be used to deliver a combination of Cas mRNA and guide RNA or a combination of Cas protein and guide RNA. LNP-mediated delivery can be used to deliver a guide RNA in the form of RNA. In a specific example, the guide RNA and the Cas protein are each introduced in the form of RNA via LNP-mediated delivery in the same LNP. As discussed in more detail elsewhere herein, one or more of the RNAs can be modified. For example, guide RNAs can be modified to comprise one or more stabilizing end modifications at the 5′ end and/or the 3′ end. Such modifications can include, for example, one or more phosphorothioate linkages at the 5′ end and/or the 3′ end or one or more 2′-O-methyl modifications at the 5′ end and/or the 3′ end. As another example, Cas mRNA modifications can include substitution with pseudouridine (e.g., fully substituted with pseudouridine), 5′ caps, and polyadenylation. As another example, Cas mRNA modifications can include substitution with N1-methyl-pseudouridine (e.g., fully substituted with N1-methyl-pseudouridine), 5′ caps, and polyadenylation. Other modifications are also contemplated as disclosed elsewhere herein. Delivery through such methods can result in transient Cas expression and/or transient presence of the guide RNA, and the biodegradable lipids improve clearance, improve tolerability, and decrease immunogenicity. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1 and WO 2017/173054 A1, each of which is herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as DSPC. In another example, the other components can comprise a helper lipid such as cholesterol, an optional neutral lipid such as DSPC, and a stealth lipid such as 5010, 5024, 5027, 5031, or 5033.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22(9):2227-2235 and WO 2017/173054 A1, each of which is herein incorporated by reference in its entirety for all purposes. In certain LNPs, the cargo can include a guide RNA or a nucleic acid encoding a guide RNA. In certain LNPs, the cargo can include an mRNA encoding a Cas nuclease, such as Cas9, and a guide RNA or a nucleic acid encoding a guide RNA. In certain LNPs, the cargo can include a F9 nucleic acid construct. In certain LNPs, the cargo can include an mRNA encoding a Cas nuclease, such as Cas9, a guide RNA or a nucleic acid encoding a guide RNA, and a F9 nucleic acid construct. LNPs for use in the methods are described in more detail elsewhere herein.

Exemplary dosing of LNPs includes about 0.1, about 0.25, about 0.3, about 0.5, about 1, about 2, about 3, about 4, about 5, about 6, about 8, or about 10 mg/kg body weight (mpk) or about 0.1 to about 10, about 0.25 to about 10, about 0.3 to about 10, about 0.5 to about 10, about 1 to about 10, about 2 to about 10, about 3 to about 10, about 4 to about 10, about 5 to about 10, about 6 to about 10, about 8 to about 10, about 0.1 to about 8, about 0.1 to about 6, about 0.1 to about 5, about 0.1 to about 4, about 0.1 to about 3, about 0.1 to about 2, about 0.1 to about 1, about 0.1 to about 0.5, about 0.1 to about 0.3, about 0.1 to about 0.25, about 0.25 to about 8, about 0.3 to about 6, about 0.5 to about 5, about 1 to about 5, or about 2 to about 3 mg/kg body weight with respect to total RNA (Cas9 mRNA and gRNA) cargo content. Such LNPs can be administered, for example, intravenously. In one example, LNP doses between about 0.01 mg/kg and about 10 mg/kg, between about 0.1 and about 10 mg/kg, or between about 0.01 and about 0.3 mg/kg can be used. For example, LNP doses of about 0.01, about 0.03, about 0.1, about 0.3, about 1, about 3, or about 10 mg/kg can be used. Additional exemplary dosing of LNPs includes about 0.1, about 0.25, about 0.3, about 0.5, about 1, about 2, about 3, about 4, about 5, about 6, about 8, or about 10 mg/kg (mpk) body weight or about 0.1 to about 10, about 0.25 to about 10, about 0.3 to about 10, about 0.5 to about 10, about 1 to about 10, about 2 to about 10, about 3 to about 10, about 4 to about 10, about 5 to about 10, about 6 to about 10, about 8 to about 10, about 0.1 to about 8, about 0.1 to about 6, about 0.1 to about 5, about 0.1 to about 4, about 0.1 to about 3, about 0.1 to about 2, about 0.1 to about 1, about 0.1 to about 0.5, about 0.1 to about 0.3, about 0.1 to about 0.25, about 0.25 to about 8, about 0.3 to about 6, about 0.5 to about 5, about 1 to about 5, or about 2 to about 3 mg/kg body weight with respect to total RNA (Cas9 mRNA and gRNA) cargo content. Such LNPs can be administered, for example, intravenously. In one example, LNP doses between about 0.01 mg/kg and about 10 mg/kg, between about 0.1 and about 10 mg/kg, or between about 0.01 and about 0.3 mg/kg can be used. For example, LNP doses of about 0.01, about 0.03, about 0.1, about 0.3, about 0.5, about 1, about 2, about 3, or about 10 mg/kg can be used. In another example, LNP doses between about 0.5 and about 10, between about 0.5 and about 5, between about 0.5 and about 3, between about 1 and about 10, between about 1 and about 5, between about 1 and about 3, or between about 1 and about 2 mg/kg can be used. In another example, LNP doses between about 0.5 and about 3, between about 0.5 and about 2.5, between about 0.5 and about 2, between about 0.5 and about 1.5, between about 0.5 and about 1, between about 1 and about 3, between about 1 and about 2.5, between about 1 and about 2, or between about 1 and about 1.5 mg/kg can be used. In another example, an LNP dose of about 1 mg/kg can be used.

The mode of delivery can be selected to decrease immunogenicity. For example, a Cas protein and a gRNA may be delivered by different modes (e.g., bi-modal delivery). These different modes may confer different pharmacodynamics or pharmacokinetic properties on the subject delivered molecule (e.g., Cas or nucleic acid encoding, gRNA or nucleic acid encoding, or F9 nucleic acid construct). For example, the different modes can result in different tissue distribution, different half-life, or different temporal distribution. Some modes of delivery (e.g., delivery of a nucleic acid vector that persists in a cell by autonomous replication or genomic integration) result in more persistent expression and presence of the molecule, whereas other modes of delivery are transient and less persistent (e.g., delivery of an RNA or a protein). Delivery of Cas proteins in a more transient manner, for example as mRNA or protein, can ensure that the Cas/gRNA complex is only present and active for a short period of time and can reduce immunogenicity caused by peptides from the bacterially-derived Cas enzyme being displayed on the surface of the cell by WIC molecules. Such transient delivery can also reduce the possibility of off-target modifications.

Administration in vivo can be by any suitable route including, for example, parenteral, intravenous, oral, subcutaneous, intra-arterial, intracranial, intrathecal, intraperitoneal, topical, intranasal, or intramuscular. Systemic modes of administration include, for example, oral and parenteral routes. Examples of parenteral routes include intravenous, intraarterial, intraosseous, intramuscular, intradermal, subcutaneous, intranasal, and intraperitoneal routes. A specific example is intravenous infusion. Nasal instillation and intravitreal injection are other specific examples. Local modes of administration include, for example, intrathecal, intracerebroventricular, intraparenchymal (e.g., localized intraparenchymal delivery to the striatum (e.g., into the caudate or into the putamen), cerebral cortex, precentral gyms, hippocampus (e.g., into the dentate gyrus or CA3 region), temporal cortex, amygdala, frontal cortex, thalamus, cerebellum, medulla, hypothalamus, tectum, tegmentum, or substantia nigra), intraocular, intraorbital, subconjuctival, intravitreal, subretinal, and transscleral routes. Significantly smaller amounts of the components (compared with systemic approaches) may exert an effect when administered locally (for example, intraparenchymal or intravitreal) compared to when administered systemically (for example, intravenously). Local modes of administration may also reduce or eliminate the incidence of potentially toxic side effects that may occur when therapeutically effective amounts of a component are administered systemically. In a specific example, administration in vivo is intravenous.

Administration in vivo can be by any suitable route including, for example, parenteral, intravenous, oral, subcutaneous, intra-arterial, intracranial, intrathecal, intraperitoneal, topical, intranasal, or intramuscular. A specific example is intravenous infusion. Compositions comprising the guide RNAs and/or Cas proteins (or nucleic acids encoding the guide RNAs and/or Cas proteins) can be formulated using one or more physiologically and pharmaceutically acceptable carriers, diluents, excipients or auxiliaries. The formulation can depend on the route of administration chosen. Pharmaceutically acceptable means that the carrier, diluent, excipient, or auxiliary is compatible with the other ingredients of the formulation and not substantially deleterious to the recipient thereof. In a specific example, the route of administration and/or formulation or chosen for delivery to the liver (e.g., hepatocytes).

The frequency of administration and the number of dosages can depend on a number of factors. The introduction of nucleic acids or proteins into the cell or subject can be performed one time or multiple times over a period of time. For example, the introduction can be performed only once over a period of time, at least two times over a period of time, at least three times over a period of time, at least four times over a period of time, at least five times over a period of time, at least six times over a period of time, at least seven times over a period of time, at least eight times over a period of time, at least nine times over a period of times, at least ten times over a period of time, at least eleven times, at least twelve times over a period of time, at least thirteen times over a period of time, at least fourteen times over a period of time, at least fifteen times over a period of time, at least sixteen times over a period of time, at least seventeen times over a period of time, at least eighteen times over a period of time, at least nineteen times over a period of time, or at least twenty times over a period of time. In some methods, a single administration of the F9 nucleic acid construct (or a single administration of the F9 nucleic acid construct and nuclease agent (e.g., Cas protein and guide RNA)) is sufficient to increase expression of FIX to a desirable level. In other methods, more than one administration may be beneficial to maximize therapeutic effect.

The methods disclosed herein can increase FIX protein levels and/or FIX activity levels in a cell or subject (e.g., circulating, serum, or plasma levels in a subject) and can comprise measuring FIX protein levels and/or activity levels in a cell or subject (e.g., circulating, serum, or plasma levels in a subject). In one example, the effectiveness of the treatment in a subject can be assessed by measuring serum or plasma FIX activity, wherein an increase in the subject's plasma level and/or activity of FIX indicates effectiveness of the treatment. In another example, effectiveness of the treatment can be determined by assessing clotting function in an aPTT assay and/or thrombin generation in an TGA-EA assay. In another example, effectiveness of the treatment can be determined by assessing the level or activity of Factor IX (e.g., circulating FIX) through a coagulation and/or an immunologic assay (e.g., a sandwich immunoassay, ELISA, or MSD).

In normal or healthy individuals, FIX activity and antigen levels vary between about 50% and 160% of normal pooled plasma, which is about 3-5 μg/mL, based on its purification from adult human plasma. See, e.g., Amiral et al. (1984) Clin. Chem. 30(9):1512-1516, herein incorporated by reference in its entirety for all purposes. In a specific example, normal FIX activity is considered to be about 100% of normal pooled plasma level of FIX activity or is considered to be 100% of normal pooled plasma level of FIX activity. In a specific example, normal plasma FIX levels are considered to be about 5 μg/mL or are considered to be 5 μg/mL. Individuals having less than 50% of normal plasma level of FIX activity and/or antigen levels are classified as having hemophilia. In particular, individuals with less than about 1% active FIX are classified as having severe hemophilia, while those with about 1-5% active FIX have moderate hemophilia. Individuals with mild hemophilia have between about 6-49% of normal levels of active clotting factor. In some embodiments, the level of circulating FIX can be measured by a coagulation and/or an immunologic assay using well known methods.

In some methods, plasma levels of FIX or FIX activity levels in a subject having hemophilia are increased to about or at least about 2%, about or at least about 3%, about or at least about 4%, about or at least about 5%, about or at least about 6%, about or at least about 7%, about or at least about 8%, about or at least about 9%, about or at least about 10%, about or at least about 11%, about or at least about 12%, about or at least about 13%, about or at least about 14%, about or at least about 15%, about or at least about 16%, about or at least about 17%, about or at least about 18%, about or at least about 19%, about or at least about 20%, about or at least about 21%, about or at least about 22%, about or at least about 23%, about or at least about 24%, about or at least about 25%, about or at least about 26%, about or at least about 27%, about or at least about 28%, about or at least about 29%, about or at least about 30%, about or at least about 31%, about or at least about 32%, about or at least about 33%, about or at least about 34%, about or at least about 35%, about or at least about 36%, about or at least about 37%, about or at least about 38%, about or at least about 39%, about or at least about 40%, about or at least about 41%, about or at least about 42%, about or at least about 43%, about or at least about 44%, about or at least about 45%, about or at least about 46%, about or at least about 47%, about or at least about 48%, about or at least about 49%, about or at least about 50%, or more, of normal level.

In some methods, circulating FIX protein levels are increased to about or at least about 0.05, about or at least about 0.1, about or at least about 0.2, about or at least about 0.5, about or at least about 1, about or at least about 2, about or at least about 3, or about or at least about 4 μg/mL. FIX protein levels may reach about 150 μg/mL, or more. In some methods, FIX protein levels are increased to at least about 4 μg/mL or about 4 μg/mL. In some methods, FIX protein levels are increased to about 4 μg/mL to about 5 μg/mL, about 4 μg/mL to 6 μg/mL, about 4 μg/mL to 8 μg/mL, about 4 μg/mL to about 10 μg/mL, or more. In some methods, FIX protein levels are increased to about 0.1 μg/mL to about 10 μg/mL, about 1 μg/mL to about 10 μg/mL, about 0.1 μg/mL to about 6 μg/mL, about 1 μg/mL to about 6 μg/mL, about 2 μg/mL to about 5 μg/mL, or about 3 μg/mL to about 5 μg/mL. For example, the compositions and methods disclosed herein are useful for increasing plasma levels of Factor IX in a subject having hemophilia to about 6, about 7, about 8, about 9, about 10, about 12, about 14, about 16, about 18, about 20, about 22, about 24, about 26, about 28, about 30, about 32, about 34, about 36, about 38, about 40, about 42, about 44, about 46, about 48, about 50, about 52, about 54, about 56, about 58, about 60, about 62, about 64, about 66, about 68, about 70, about 75, about 80, about 85, about 90, about 95, about 100, about 105, about 110, about 115, about 120, about 125, about 130, about 135, about 140, about 145, about 150 μg/mL, or more.

In some methods, plasma FIX activity and/or levels in a subject (e.g., having hemophilia) are increased by about or at least about 1%, about or at least about 2%, about or at least about 3%, about or at least about 4%, about or at least about 5%, about or at least about 6%, about or at least about 7%, about or at least about 8%, about or at least about 9%, about or at least about 10%, about or at least about 11%, about or at least about 12%, about or at least about 13%, about or at least about 14%, about or at least about 15%, about or at least about 16%, about or at least about 17%, about or at least about 18%, about or at least about 19%, about or at least about 20%, about or at least about 21%, about or at least about 22%, about or at least about 23%, about or at least about 24%, about or at least about 25%, about or at least about 26%, about or at least about 27%, about or at least about 28%, about or at least about 29%, about or at least about 30%, about or at least about 31%, about or at least about 32%, about or at least about 33%, about or at least about 34%, about or at least about 35%, about or at least about 36%, about or at least about 37%, about or at least about 38%, about or at least about 39%, about or at least about 40%, about or at least about 41%, about or at least about 42%, about or at least about 43%, about or at least about 44%, about or at least about 45%, about or at least about 46%, about or at least about 47%, about or at least about 48%, about or at least about 49%, about or at least about 50%, about or at least about 55%, about or at least about 60%, about or at least about 65%, about or at least about 70%, about or at least about 75%, about or at least about 80%, about or at least about 85%, about or at least about 90%, about or at least about 95%, about or at least about 100%, about or at least about 110%, about or at least about 120%, about or at least about 130%, about or at least about 140%, about or at least about 150%, about or at least about 160%, about or at least about 170%, about or at least about 180%, about or at least about 190%, about or at least about 200%, or more, as compared to the subject's plasma level and/or activity of FIX before administration.

In some methods, FIX activity and/or protein levels in a cell or population of cells (e.g., liver cells, or hepatocytes) are increased by about or at least about 1%, about or at least about 2%, about or at least about 3%, about or at least about 4%, about or at least about 5%, about or at least about 6%, about or at least about 7%, about or at least about 8%, about or at least about 9%, about or at least about 10%, about or at least about 11%, about or at least about 12%, about or at least about 13%, about or at least about 14%, about or at least about 15%, about or at least about 16%, about or at least about 17%, about or at least about 18%, about or at least about 19%, about or at least about 20%, about or at least about 21%, about or at least about 22%, about or at least about 23%, about or at least about 24%, about or at least about 25%, about or at least about 26%, about or at least about 27%, about or at least about 28%, about or at least about 29%, about or at least about 30%, about or at least about 31%, about or at least about 32%, about or at least about 33%, about or at least about 34%, about or at least about 35%, about or at least about 36%, about or at least about 37%, about or at least about 38%, about or at least about 39%, about or at least about 40%, about or at least about 41%, about or at least about 42%, about or at least about 43%, about or at least about 44%, about or at least about 45%, about or at least about 46%, about or at least about 47%, about or at least about 48%, about or at least about 49%, about or at least about 50%, about or at least about 55%, about or at least about 60%, about or at least about 65%, about or at least about 70%, about or at least about 75%, about or at least about 80%, about or at least about 85%, about or at least about 90%, about or at least about 95%, about or at least about 100%, about or at least about 110%, about or at least about 120%, about or at least about 130%, about or at least about 140%, about or at least about 150%, about or at least about 160%, about or at least about 170%, about or at least about 180%, about or at least about 190%, about or at least about 200%, or more, as compared to the FIX activity and/or protein levels before administration (e.g., a normal level).

Some methods comprise expressing a therapeutically effective amount of FIX protein (e.g., achieving a therapeutically effective level of circulating FIX coagulation activity in an individual). Some methods comprise achieving FIX activity or expression levels of at least about 5% to about 50% of normal or at least about 50% to about 150% of normal. Some methods comprise achieving an increase in FIX activity or expression over the patient's baseline FIX activity or expression of at least about 1% to about 50% of normal FIX activity, or at least about 5% to about 50% of normal FIX activity, or at least about 50% to about 150% of normal FIX activity. Some methods comprise achieving FIX activity or expression levels of between about 40% and about 150% of normal (i.e., between 40% and 150% of normal). Some methods comprise achieving FIX activity or expression levels of between about 40% and about 100% of normal (e.g., between 40% and 100% of normal). Some methods comprise achieving FIX activity levels of between about 40% and about 150% of normal (i.e., between 40% and 150% of normal). Some methods comprise achieving FIX activity levels of between about 40% and about 100% of normal (e.g., between 40% and 100% of normal). Some methods comprise achieving FIX expression levels of between about 40% and about 150% of normal (i.e., between 40% and 150% of normal). Some methods comprise achieving FIX expression levels of between about 40% and about 100% of normal (e.g., between 40% and 100% of normal).

In a specific example, the FIX activity levels in a subject are increased to between about 1% to about 300%, about 1% to about 250%, about 1% to about 200%, about 1% to about 190%, about 1% to about 180%, about 1% to about 170%, about 1% to about 160%, about 1% to about 150%, about 1% to about 140%, about 1% to about 130%, about 1% to about 120%, about 1% to about 110%, about 1% to about 100%, about 1% to about 90%, about 1% to about 80%, about 1% to about 70%, about 1% to about 60%, about 1% to about 50%, about 5% to about 300%, about 5% to about 250%, about 5% to about 200%, about 5% to about 190%, about 5% to about 180%, about 5% to about 170%, about 5% to about 160%, about 5% to about 150%, about 5% to about 140%, about 5% to about 130%, about 5% to about 120%, about 5% to about 110%, about 5% to about 100%, about 5% to about 90%, about 5% to about 80%, about 5% to about 70%, about 5% to about 60%, about 5% to about 50%, about 15% to about 300%, about 15% to about 250%, about 15% to about 200%, about 15% to about 190%, about 15% to about 180%, about 15% to about 170%, about 15% to about 160%, about 15% to about 150%, about 15% to about 140%, about 15% to about 130%, about 15% to about 120%, about 15% to about 110%, about 15% to about 100%, about 15% to about 90%, about 15% to about 80%, about 15% to about 70%, about 15% to about 60%, about 15% to about 50%, about 10% to about 300%, about 15% to about 300%, about 20% to about 300%, about 25% to about 300%, about 30% to about 300%, about 35% to about 300%, about 40% to about 300%, about 45% to about 300%, about 50% to about 300%, about 55% to about 300%, about 60% to about 300%, about 65% to about 300%, about 70% to about 300%, about 75% to about 300%, about 80% to about 300%, about 85% to about 300%, about 90% to about 300%, about 95% to about 300%, about 100% to about 300%, about 10% to about 200%, about 15% to about 200%, about 20% to about 200%, about 25% to about 200%, about 30% to about 200%, about 35% to about 200%, about 40% to about 200%, about 45% to about 200%, about 50% to about 200%, about 55% to about 200%, about 60% to about 200%, about 65% to about 200%, about 70% to about 200%, about 75% to about 200%, about 80% to about 200%, about 85% to about 200%, about 90% to about 200%, about 95% to about 200%, about 100% to about 200%, about 10% to about 150%, about 15% to about 150%, about 20% to about 150%, about 25% to about 150%, about 30% to about 150%, about 35% to about 150%, about 40% to about 150%, about 45% to about 150%, about 50% to about 150%, about 55% to about 150%, about 60% to about 150%, about 65% to about 150%, about 70% to about 150%, about 75% to about 150%, about 80% to about 150%, about 85% to about 150%, about 90% to about 150%, about 95% to about 150%, about 100% to about 150%, about 50% to about 300%, about 50% to about 250%, about 50% to about 200%, about 50% to about 190%, about 50% to about 180%, about 50% to about 170%, about 50% to about 160%, about 50% to about 150%, about 50% to about 140%, about 50% to about 130%, about 50% to about 120%, about 50% to about 110%, about 50% to about 100% of normal FIX activity levels (e.g., between about 50% to about 150% of normal FIX activity levels).

In a specific example, the FIX activity levels in a subject are increased to between about 5% to about 200%, about 10% to about 190%, about 20% to about 180%, about 30% to about 170%, about 40% to about 160%, about 50% to about 150%, about 60% to about 140%, about 70% to about 130%, about 80% to about 120%, or about 90% to about 110% of normal FIX activity levels (e.g., to or about 100% of normal FIX activity levels).

In a specific example, the plasma FIX levels in a subject are increased to between about 1% to about 300%, about 1% to about 250%, about 1% to about 200%, about 1% to about 190%, about 1% to about 180%, about 1% to about 170%, about 1% to about 160%, about 1% to about 150%, about 1% to about 140%, about 1% to about 130%, about 1% to about 120%, about 1% to about 110%, about 1% to about 100%, about 1% to about 90%, about 1% to about 80%, about 1% to about 70%, about 1% to about 60%, about 1% to about 50%, about 5% to about 300%, about 5% to about 250%, about 5% to about 200%, about 5% to about 190%, about 5% to about 180%, about 5% to about 170%, about 5% to about 160%, about 5% to about 150%, about 5% to about 140%, about 5% to about 130%, about 5% to about 120%, about 5% to about 110%, about 5% to about 100%, about 5% to about 90%, about 5% to about 80%, about 5% to about 70%, about 5% to about 60%, about 5% to about 50%, about 15% to about 300%, about 15% to about 250%, about 15% to about 200%, about 15% to about 190%, about 15% to about 180%, about 15% to about 170%, about 15% to about 160%, about 15% to about 150%, about 15% to about 140%, about 15% to about 130%, about 15% to about 120%, about 15% to about 110%, about 15% to about 100%, about 15% to about 90%, about 15% to about 80%, about 15% to about 70%, about 15% to about 60%, about 15% to about 50%, about 10% to about 300%, about 15% to about 300%, about 20% to about 300%, about 25% to about 300%, about 30% to about 300%, about 35% to about 300%, about 40% to about 300%, about 45% to about 300%, about 50% to about 300%, about 55% to about 300%, about 60% to about 300%, about 65% to about 300%, about 70% to about 300%, about 75% to about 300%, about 80% to about 300%, about 85% to about 300%, about 90% to about 300%, about 95% to about 300%, about 100% to about 300%, about 10% to about 200%, about 15% to about 200%, about 20% to about 200%, about 25% to about 200%, about 30% to about 200%, about 35% to about 200%, about 40% to about 200%, about 45% to about 200%, about 50% to about 200%, about 55% to about 200%, about 60% to about 200%, about 65% to about 200%, about 70% to about 200%, about 75% to about 200%, about 80% to about 200%, about 85% to about 200%, about 90% to about 200%, about 95% to about 200%, about 100% to about 200%, about 10% to about 150%, about 15% to about 150%, about 20% to about 150%, about 25% to about 150%, about 30% to about 150%, about 35% to about 150%, about 40% to about 150%, about 45% to about 150%, about 50% to about 150%, about 55% to about 150%, about 60% to about 150%, about 65% to about 150%, about 70% to about 150%, about 75% to about 150%, about 80% to about 150%, about 85% to about 150%, about 90% to about 150%, about 95% to about 150%, about 100% to about 150%, about 50% to about 300%, about 50% to about 250%, about 50% to about 200%, about 50% to about 190%, about 50% to about 180%, about 50% to about 170%, about 50% to about 160%, about 50% to about 150%, about 50% to about 140%, about 50% to about 130%, about 50% to about 120%, about 50% to about 110%, about 50% to about 100% of normal plasma FIX levels (e.g., between about 50% to about 150% of normal plasma FIX levels).

In a specific example, the plasma FIX levels in a subject are increased to between about 5% to about 200%, about 10% to about 190%, about 20% to about 180%, about 30% to about 170%, about 40% to about 160%, about 50% to about 150%, about 60% to about 140%, about 70% to about 130%, about 80% to about 120%, or about 90% to about 110% of normal plasma FIX levels (e.g., to or about 100% of normal plasma FIX levels).

In a specific example, the plasma FIX levels in a subject are increased to between about 0.25 μg/mL to about 15 μg/mL, about 0.25 μg/mL to about 14 μg/mL, about 0.25 μg/mL to about 13 μg/mL, about 0.25 μg/mL to about 12 μg/mL, about 0.25 μg/mL to about 11 μg/mL, about 0.25 μg/mL to about 10 μg/mL, about 0.25 μg/mL to about 9 μg/mL, about 0.25 μg/mL to about 8 μg/mL, about 0.25 μg/mL to about 7 μg/mL, about 0.25 μg/mL to about 6 μg/mL, about 0.25 μg/mL to about 5 μg/mL, about 0.25 μg/mL to about 4 μg/mL, about 0.25 μg/mL to about 3 μg/mL, about 0.25 μg/mL to about 2 μg/mL, about 0.25 μg/mL to about 1 μg/mL, about 0.75 μg/mL to about 15 μg/mL, about 0.75 μg/mL to about 14 μg/mL, about 0.75 μg/mL to about 13 μg/mL, about 0.75 μg/mL to about 12 μg/mL, about 0.75 μg/mL to about 11 μg/mL, about 0.75 μg/mL to about 10 μg/mL, about 0.75 μg/mL to about 9 μg/mL, about 0.75 μg/mL to about 8 μg/mL, about 0.75 μg/mL to about 7 μg/mL, about 0.75 μg/mL to about 6 μg/mL, about 0.75 μg/mL to about 5 μg/mL, about 0.75 μg/mL to about 4 μg/mL, about 0.75 μg/mL to about 3 μg/mL, about 0.75 μg/mL to about 2 μg/mL, about 0.75 μg/mL to about 1 μg/mL, about 0.5 μg/mL to about 15 μg/mL, about 0.75 μg/mL to about 15 μg/mL, about 1 μg/mL to about 15 μg/mL, about 2 μg/mL to about 15 μg/mL, about 3 μg/mL to about 15 μg/mL, about 4 μg/mL to about 15 μg/mL, about 5 μg/mL to about 15 μg/mL, about 0.5 μg/mL to about 10 μg/mL, about 0.75 μg/mL to about 10 μg/mL, about 1 μg/mL to about 10 μg/mL, about 2 μg/mL to about 10 μg/mL, about 3 μg/mL to about 10 μg/mL, about 4 μg/mL to about 10 μg/mL, about 5 μg/mL to about 10 μg/mL, about 0.5 μg/mL to about 7.5 μg/mL, about 0.75 μg/mL to about 7.5 μg/mL, about 1 μg/mL to about 7.5 μg/mL, about 2 μg/mL to about 7.5 μg/mL, about 2.5 μg/mL to about 7.5 μg/mL, about 3 μg/mL to about 7.5 μg/mL, about 4 μg/mL to about 7.5 μg/mL, about 5 μg/mL to about 7.5 μg/mL, about 2.5 μg/mL to about 15 μg/mL, about 2.5 μg/mL to about 14 μg/mL, about 2.5 μg/mL to about 13 μg/mL, about 2.5 μg/mL to about 12 μg/mL, about 2.5 μg/mL to about 11 μg/mL, about 2.5 μg/mL to about 10 μg/mL, about 2.5 μg/mL to about 9 μg/mL, about 2.5 μg/mL to about 8 μg/mL, about 2.5 μg/mL to about 7 μg/mL, about 2.5 μg/mL to about 6 μg/mL, about 2.5 μg/mL to about 5 μg/mL, about 3 μg/mL to about 15 μg/mL, about 3 μg/mL to about 14 μg/mL, about 3 μg/mL to about 13 μg/mL, about 3 μg/mL to about 12 μg/mL, about 3 μg/mL to about 11 μg/mL, about 3 μg/mL to about 10 μg/mL, about 3 μg/mL to about 9 μg/mL, about 3 μg/mL to about 8 μg/mL, about 3 μg/mL to about 7 μg/mL, about 3 μg/mL to about 6 μg/mL, about 3 μg/mL to about 5 μg/mL (e.g., between about 2.5 μg/mL to about 7.5 μg/mL or between about 3 μg/mL to about 5 μg/mL). In some embodiments, the recited expression levels are at least 1 month after administration. In some embodiments, the recited expression levels are at least 2 months after administration. In some embodiments, the recited expression levels are at least 3 months after administration. In some embodiments, the recited expression levels are at least 4 months after administration. In some embodiments, the recited expression levels are at least 5 months after administration. In some embodiments, the recited expression levels are at least 6 months after administration. In some embodiments, the recited expression levels are at least 9 months after administration. In some embodiments, the recited expression levels are at least 12 months after administration.

In a specific example, the plasma FIX levels in a subject are increased to between about 0.5 μg/mL to about 10 μg/mL, about 1 μg/mL to about 9 μg/mL, about 2 μg/mL to about 8 μg/mL, about 3 μg/mL to about 7 μg/mL, or about 4 μg/mL to about 6 μg/mL (e.g., to about 5 μg/mL). In some embodiments, the recited expression levels are at least 1 month after administration. In some embodiments, the recited expression levels are at least 2 months after administration. In some embodiments, the recited expression levels are at least 3 months after administration. In some embodiments, the recited expression levels are at least 4 months after administration. In some embodiments, the recited expression levels are at least 5 months after administration. In some embodiments, the recited expression levels are at least 6 months after administration. In some embodiments, the recited expression levels are at least 9 months after administration. In some embodiments, the recited expression levels are at least 12 months after administration.

In a specific example, the FIX activity levels in a subject are increased to at least about 15% of normal FIX activity levels. In a specific example, the plasma FIX levels in a subject are increased to at least about 15% of normal plasma FIX levels. In a specific example, the plasma FIX levels in a subject are increased to at least about 0.75 μg/mL. For example, the method can be a method of preventing or inhibiting spontaneous bleeding in a subject having hemophilia B, and the FIX activity levels in a subject are increased to at least about 15% of normal FIX activity levels or the plasma FIX levels in a subject are increased to at least about 15% of normal plasma FIX levels or the plasma FIX levels in a subject are increased to at least about 0.75 μg/mL.

In a specific example, the FIX activity levels in a subject are increased to no more than about 300%, no more than about 250%, no more than about 200%, or no more than about 150% of normal FIX activity levels. In a specific example, the plasma FIX levels in a subject are increased to no more than about 300%, no more than about 250%, no more than about 200%, or no more than about 150% of normal plasma FIX levels.

In a specific example, a subject has severe hemophilia, and the FIX activity levels in the subject are increased to at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal FIX activity levels.

In a specific example, a subject has severe hemophilia, and the plasma FIX levels in the subject are increased to at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal plasma FIX levels.

In a specific example, a subject has severe hemophilia, and the FIX activity levels in the subject are increased to more than about 1%, more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal FIX activity levels.

In a specific example, a subject has severe hemophilia, and the plasma FIX levels in the subject are increased to more than about 1%, more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal plasma FIX levels.

In a specific example, a subject has moderate hemophilia, and the FIX activity levels in the subject are increased to at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal FIX activity levels.

In a specific example, a subject has moderate hemophilia, and the plasma FIX levels in the subject are increased to at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal plasma FIX levels.

In a specific example, a subject has moderate hemophilia, and the FIX activity levels in the subject are increased to more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal FIX activity levels.

In a specific example, a subject has moderate hemophilia, and the plasma FIX levels in the subject are increased to more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal plasma FIX levels.

In a specific example, a subject has mild hemophilia, and the FIX activity levels in the subject are increased to at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal FIX activity levels.

In a specific example, a subject has mild hemophilia, and the plasma FIX levels in the subject are increased to at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal plasma FIX levels.

In a specific example, a subject has mild hemophilia, and the FIX activity levels in the subject are increased to more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal FIX activity levels.

In a specific example, a subject has mild hemophilia, and the plasma FIX levels in the subject are increased to more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal plasma FIX levels.

Some methods comprise achieving a durable effect, such as an at least 1 month, at least 2 months, at least 6 months, at least 1 year, or at least 2 year effect. Some methods comprise achieving the therapeutic effect in a durable and sustained manner, such as an at least 1 month, at least 2 months, at least 6 months, at least 1 year, or at least 2 year effect. In some methods, the increased circulating FIX activity and/or expression level is stable for at least 1 month, at least 2 months, at least 6 months, at least 1 year, or more. In some methods, a steady-state activity and/or level of FIX protein is achieved by at least 7 days, at least 14 days, or at least 28 days. In additional methods, the method comprises maintaining FIX activity and/or levels after a single dose for at least 1, at least 2, at least 4, or at least 6 months, or at least 1, at least 2, at least 3, at least 4, or at least 5 years. Some methods comprise achieving a durable or sustained effect in a human, such as an at least at least 8 weeks, at least 24 weeks, for example, at least 1 year (52 weeks), or optionally at least 2 year effect, and in some embodiments, at least 3 year, at least 4 year, or at least 5 year effect. Some methods comprise achieving the therapeutic effect in a human in a durable and sustained manner, such as an at least 8 weeks, at least 24 weeks, for example, at least 1 year, or optionally at least 2 year effect, and in some embodiments, at least 3 year, at least 4 year, or at least 5 year effect. In some methods, the increased FIX activity and/or expression level in a human is stable for at least at least 8 weeks, at least 24 weeks, for example, at least 1 year, optionally at least 2 years, and in some embodiments, at least 3 years, at least 4 years, or at least 5 years. In some methods, a steady-state activity and/or level of FIX in a human is achieved by at least 7 days, at least 14 days, or at least 28 days, optionally at least 56 days, at least 80 days, or at least 96 days. In additional methods, the method comprises maintaining FIX activity and/or levels after a single dose in a human for at least 8 weeks, at least 16 weeks, or at least 24 week, or in some embodiments at least 1 year, or at least 2 years, optionally at least 3 years, at least 4 years, or at least 5 years. For example, expression of the FIX can be sustained in the human subject for at least about 8 weeks, at least about 12 weeks, at least about 24 weeks, in certain embodiments, at least about 1 year, or at least about 2 years after treatment, and in some embodiments, at least 3 years, at least 4 years, or at least 5 years after treatment. Likewise, activity of the FIX can be sustained in the human subject for at least about 8 weeks, at least about 12 weeks, at least about 24 weeks, in certain embodiments for at least about 1 year, or at least about 2 years after treatment, and in some embodiments, at least 3 years, at least 4 years, or at least 5 years after treatment. In some methods, expression or activity of the FIX is maintained at a level higher than the expression or activity of the FIX prior to treatment (i.e., the subject's baseline). In some methods, expression or activity of the FIX is considered sustained if it is maintained at a therapeutically effective level of expression or activity. Relative durations, in other organisms, are understood based, e.g., on life span and developmental stages, are covered within the disclosure above. In some methods, expression or activity of the FIX is considered “sustained” if the expression or activity in a human at six months after administration, one year after administration, or two years after administration, the expression or activity is at least 50% of the expression or activity of the peak level of expression or activity measured for that subject. In certain embodiments, at six months, e.g., 24 weeks to 28 weeks, after administration the expression or activity is at least 50%, 55%, 60%, 65%, 70%, 75% or 80% of the expression or activity of the peak level of expression or activity measured for that subject. In certain embodiments, at one year, i.e., about 12 months, e.g., 11-13 months, after administration the expression or activity is at least 50%, 55%, 60%, 65%, 70%, 75% or 80% of the expression or activity of the peak level of expression or activity measured for that subject. In certain embodiments, at two years, i.e., about 24 months, e.g., 23-25 months, after administration the expression or activity is at least 50%, 55%, 60%, 65%, 70%, 75% or 80% of the expression or activity of the peak level of expression or activity measured for that subject. In certain embodiments, at six months after administration the expression or activity is at least 50%, preferably at least 60% of the expression or activity of the peak level of expression or activity measured for that subject. In certain embodiments, at one year after administration the expression or activity is at least 50%, preferably at least 60% of the expression or activity of the peak level of expression or activity measured for that subject. In certain embodiments, at two years after administration the expression or activity is at least 50%, preferably at least 60% of the expression or activity of the peak level of expression or activity measured for that subject. In preferred embodiments, the subject has routine monitoring of expression or activity levels of the FIX, e.g., weekly, monthly, particularly early after administration, e.g., within the first six months. Periodic measurements may establish that the effect on expression or activity is sustained at, e.g. 6 months after administration, one year after administration, or two years after administration. In some methods in neonatal subjects, the expression of the FIX is sustained when the neonatal subject becomes an adult. In some methods, the expression of the FIX is sustained for the lifetime of the subject or neonatal subject.

In some methods, the expression or activity of the FIX is at least 50% of the expression or activity of the FIX at a peak level of expression measured for the human subject at 24 weeks after the administering. In some methods, the expression or activity of the FIX is at least 50% of the expression or activity of the FIX at a peak level of expression measured for the human subject at one year after the administering. In some methods, the expression or activity of the FIX is at least 60% of the expression or activity of the FIX at a peak level of expression measured for the human subject at 24 weeks after the administering. In some methods, expression or activity of the FIX is at least 50% of the expression or activity of the FIX at a peak level of expression measured for the human subject at two years after the administering. In some methods, the expression or activity of the FIX is at least 60% of the expression or activity of the FIX at a peak level of expression measured for the human subject at 2 years after the administering. In some methods, the expression or activity of the FIX is at least 60% of the expression or activity of the FIX at a peak level of expression measured for the human subject at 24 weeks after the administering.

In some methods involving insertion into an ALB locus, the subject's circulating albumin levels or cell's albumin levels are normal. Such methods may comprise maintaining the subject's circulating albumin levels or the cell's albumin levels within ±5%, ±10%, ±15%, ±20%, or ±50% of normal circulating albumin levels or normal albumin levels. In some methods, the subject's or cell's albumin levels are unchanged as compared to the albumin levels of untreated individuals by at least week 4, at least week 8, at least week 12, or at least week 20. In some methods, the subject's or cell's albumin levels transiently drop and then return to normal levels. In particular, the methods may comprise detecting no significant alterations in levels of plasma albumin.

In some methods, combination therapies are used comprising the any of the compositions for expressing FIX disclosed herein together with an additional therapy suitable for treating hemophilia B or a FIX deficiency. As one example, the methods of described herein can be combined with the use of other hemostatic agents, blood factors, and medications. For example, the subject may be administered a therapeutically effective amount of one or more factors selected from the group consisting of factor XI, factor XII, prekallikrein, high molecular weight kininogen (HMWK), factor V, factor VII, factor VIII, factor X, factor XIII, factor II, factor VIIa, and von Willebrands factor. Additionally or alternatively, treatment may further comprise administering a procoagulant, such as an activator of the intrinsic coagulation pathway, including factor Xa, factor IXa, factor XIa, factor XIIa, and VIIIa, prekallekrein, and high-molecular weight kininogen; or an activator of the extrinsic coagulation pathway, including tissue factor, factor VIIa, factor Va, and factor Xa.

In some methods, the method further comprises assessing preexisting anti-AAV (e.g., anti-AAV8) immunity in a subject prior to administering any of the F9 nucleic acid constructs described herein. For example, such methods could comprise assessing immunogenicity using a total antibody (TAb) immune assay or a neutralizing antibody (NAb) assay. See, e.g., Manno et al. (2006) Nat. Med. 12(3):342-347, Kruzik et al. (2019) Mol. Ther. Methods Clin. Dev. 14:126-133, and Weber (2021) Front. Immunol. 12:658399, each of which is herein incorporated by reference in its entirety for all purposes. In some embodiments, TAb assays look for antibodies that bind to the AAV vector, whereas NAb assays assess whether the antibodies that are present stop the AAV vector from transducing target cells. With TAb assays, the drug product or an empty capsid can be used to capture the antibodies; NAb assays can require a reporter vector (e.g., a version of the AAV vector encoding luciferase).

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

TABLE 9
Description of Sequences.

| SEQ | | |
|---|---|---|
| ID | | |
| NO | Type | Description |
| 1 | Protein | Human Factor IX Protein NCBI Accession No. NP_000124.1 |
| 2 | DNA | Human F9 mRNA (cDNA) NCBI Accession No. NM_000133.4 |
| 3 | DNA | Human F9 CDS CCDS ID CCDS 14666.1 |
| 4 | DNA | Human ALB Intron 1 |
| 5 | DNA | Guide RNA Target Sequence Plus PAM v1 |
| 6 | DNA | Guide RNA Target Sequence Plus PAM v2 |
| 7 | DNA | Guide RNA Target Sequence Plus PAM v3 |
| 8 | Protein | SpCas9 Protein VI |
| 9 | DNA | SpCas9 DNA VI |
| 10 | DNA | SpCas9 mRNA (cDNA) |
| 11 | Protein | SpCas9 Protein V2 |
| 12 | RNA | SpCas9 mRNA V2 |
| 13 | Protein | SV40NLSvl |
| 14 | Protein | SV40 NLS v2 |
| 15 | Protein | Nucleoplasmin NLS |
| 16 | RNA | crRNA Tail v1 |
| 17 | RNA | crRNA Tail v2 |
| 18 | RNA | TracrRNA vl |
| 19 | RNA | TracrRNA v2 |
| 20 | RNA | TracrRNA v3 |
| 21 | RNA | gRNA Scaffold vl |
| 22 | RNA | gRNA Scaffold v2 |
| 23 | RNA | gRNA Scaffold v3 |
| 24 | RNA | gRNA Scaffold v4 |
| 25 | RNA | gRNA Scaffold v5 |
| 26 | RNA | gRNA Scaffold v6 |
| 27 | RNA | gRNA Scaffold v7 |
| 28 | RNA | gRNA Scaffold v8 |
| 29 | RNA | Modified gRNA Scaffold |
| 30-61 | RNA | Human ALB Intron 1 Guide Sequences |
| 62-125 | RNA | Human ALB Intron 1 Guide Sequences |
| 126-157 | DNA | Human ALB Intron 1 Guide RNA Target Sequences |
| 158 | DNA | Native F9 Insert |
| 159 | DNA | Native CpG removed no splice F9 Insert |
| 160 | DNA | Native CpG removed F9 Insert |
| 161 | DNA | Native splice removed F9 Insert |
| 162 | DNA | Codon optimized F9 Insert |
| 163 | DNA | COMP F9 Insert |
| 164 | DNA | DC F9 Insert |
| 165 | DNA | GA F9 Insert |
| 166 | DNA | CpG0 F9 Insert |
| 167 | DNA | CpG3 F9 Insert |
| 168 | DNA | CpG10 no CpG F9 Insert |
| 169 | DNA | CpG10 F9 Insert |
| 170 | DNA | CpG20 no CpG F9 Insert |
| 171 | DNA | CpG20 F9 Insert |
| 172 | DNA | Insert 72 |
| 173 | DNA | Insert 18 |
| 174 | DNA | Insert 19 |
| 175 | DNA | Insert 20 |
| 176 | DNA | Insert 21 |
| 177 | DNA | Insert 27 |
| 178 | DNA | Insert 28 |
| 179 | DNA | Insert 29 |
| 180 | DNA | Insert 30 |
| 181 | DNA | Insert 36 |
| 182 | DNA | Insert 37 |
| 183 | DNA | Insert 38 |
| 184 | DNA | Insert 39 |
| 185 | DNA | Insert 22 |
| 186 | DNA | Insert 23 |
| 187 | DNA | Insert 24 |
| 188 | DNA | Insert 25 |
| 189 | DNA | Insert 26 |
| 190 | DNA | Insert 31 |
| 191 | DNA | Insert 32 |
| 192 | DNA | Insert 33 |
| 193 | DNA | Insert 34 |
| 194 | DNA | Insert 35 |
| 195 | Protein | FIX Encoded by F9 Inserts |
| 196 | DNA | ITR 145 |
| 197 | DNA | ITR 141 |
| 198 | DNA | ITR 130 |
| 199 | DNA | SV40 poly A |
| 200 | DNA | CpG depleted bGH polyA |
| 201 | DNA | Mouse Alb exon 2 Splice Acceptor |
| 202 | DNA | Insert 72 no ITRs |
| 203 | DNA | Insert 18 no ITRs |
| 204 | DNA | Insert 19 no ITRs |
| 205 | DNA | Insert 20 no ITRs |
| 206 | DNA | Insert 21 no ITRs |
| 207 | DNA | Insert 27 no ITRs |
| 208 | DNA | Insert 28 no ITRs |
| 209 | DNA | Insert 29 no ITRs |
| 210 | DNA | Insert 30 no ITRs |
| 211 | DNA | Insert 36 no ITRs |
| 212 | DNA | Insert 37 no ITRs |
| 213 | DNA | Insert 38 no ITRs |
| 214 | DNA | Insert 39 no ITRs |
| 215 | DNA | Insert 22 no ITRs |
| 216 | DNA | Insert 23 no ITRs |
| 217 | DNA | Insert 24 no ITRs |
| 218 | DNA | Insert 25 no ITRs |
| 219 | DNA | Insert 26 no ITRs |
| 220 | DNA | Insert 31 no ITRs |
| 221 | DNA | Insert 32 no ITRs |
| 222 | DNA | Insert 33 no ITRs |
| 223 | DNA | Insert 34 no ITRs |
| 224 | DNA | Insert 35 no ITRs |
| 225 | RNA | Cas9 mRNA |
| 226 | RNA | Cas9 mRNA CDS |
| 227 | DNA | Cas9 CDS |
| 228 | RNA | Mouse Alb Intron 1 Guide Sequence g666 |
| 229 | DNA | Mouse Alb Intron 1 Guide RNA Target Sequence g666 |
| 230-231 | RNA | Mouse Alb Intron 1 sgRNA Sequences g666 |

### EXAMPLES

### Example 1. Single Guide RNA and DNA Template Selection

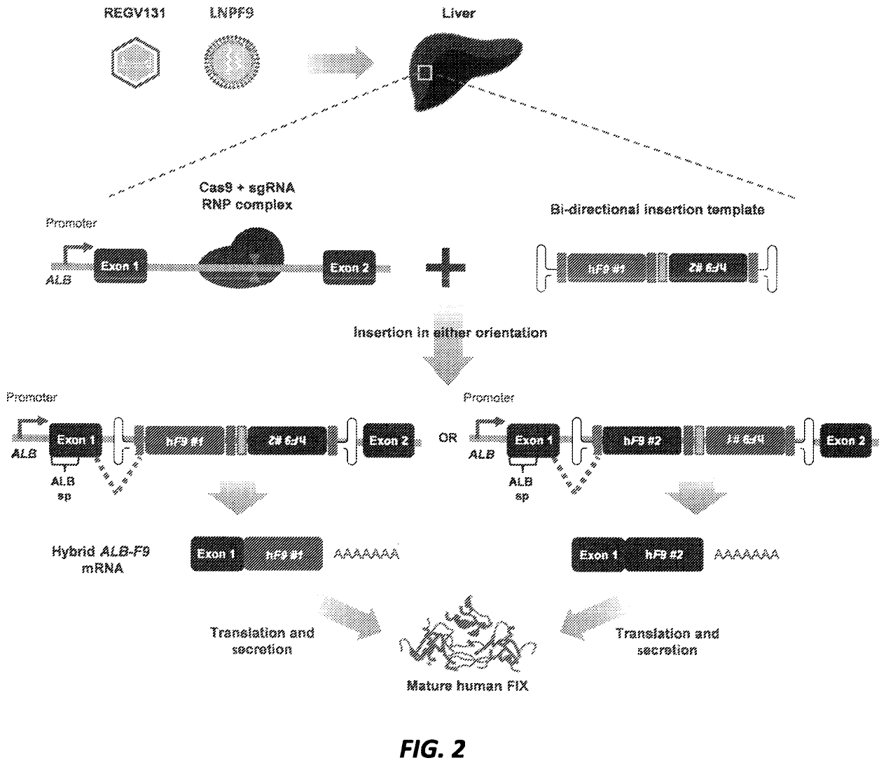

Hemophilia B is a genetic disorder caused by missing or defective Factor 9 (F9) gene and Factor IX (FIX) protein, resulting in a bleeding disorder. Hemophilia B patients display a reduced ability to clot. Described herein is an approach to treating hemophilia B using targeted transgene insertion in the albumin (ALB) locus to restore FIX protein in the plasma. The approach is based on the CRISPR/Cas9 gene editing technology contained in a lipid nanoparticle (LNP) delivery system, associated with a bidirectional F9 DNA gene insertion template contained in a recombinant adeno-associated virus serotype 8 (rAAV8). An alternate approach is based on the CRISPR/Cas9 gene editing technology contained in a lipid nanoparticle (LNP) delivery system, associated with a unidirectional F9 DNA gene insertion template contained in a recombinant adeno-associated virus serotype 8 (rAAV8). The CRISPR/Cas9 component was designed to target and cut the double stranded DNA at the ALB gene locus in hepatocytes, allowing for the F9 DNA template, delivered by AAV vectors, to be inserted in the genome at a safe harbor locus. See FIG. 2. Transgene insertion provides a functional F9 gene, encoding the missing or defective genomic F9 in hemophilia B patients. The gene insertion platform described herein can restore normal levels of FIX in plasma, for the lifetime of the patient, after a single one-time dose. The gene insertion platform described herein may overcome several of the existing limitations of AAV episome gene therapies.

AAV vectors are potent delivery vehicles of therapeutic transgenes to cells in vivo. In particular, several AAV serotypes, including AAV serotype 8 (AAV8), are capable of targeting liver hepatocytes when injected systemically in mice, non-human primates, and human patients. As such, disorders caused by deficiencies in liver-expressed genes are potential candidates for AAV-based gene therapies. One such disease is hemophilia B, which is caused by a deficiency or mutation in FIX protein, encoded by the F9 gene.

The F9 gene is exclusively expressed in hepatocytes, resulting in synthesis of the FIX protein by the liver. Therefore, a targeted gene insertion platform was created whereby a promoterless F9-encoding DNA is integrated permanently into the host cell DNA at a specific safe harbor site following the formation of a CRISPR/Cas9-induced double strand break (DSB).

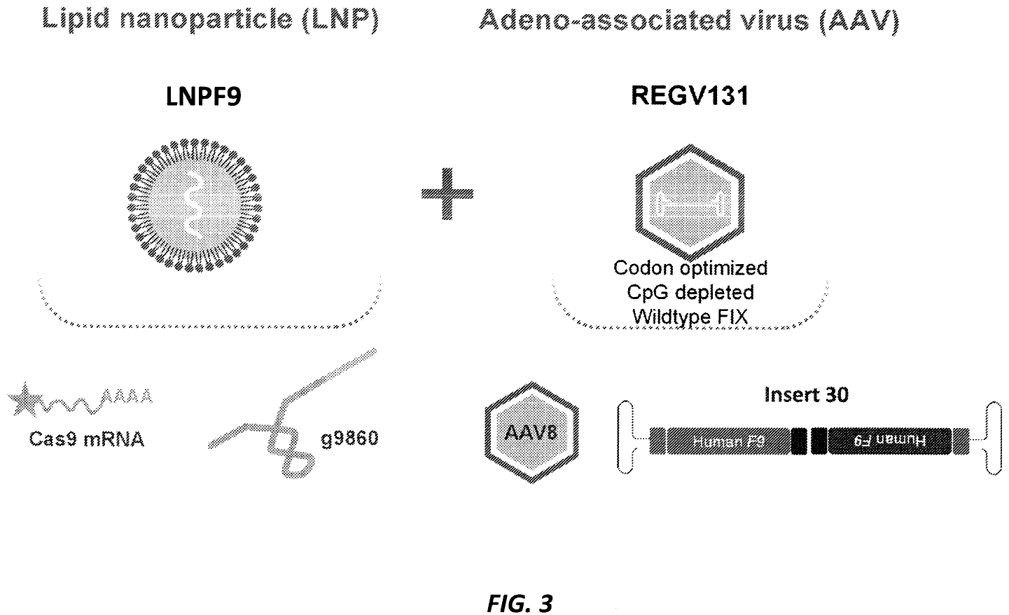

The F9 DNA template is brought into the cell by a rAAV8 vector, and the CRISPR/Cas9 RNA components (Cas9 mRNA and sgRNA) are delivered to the hepatocyte by LNP-mediated delivery (FIG. 3). The rAAV-delivered F9 DNA cassette is promoterless and instead harnesses transcription of the insertion locus to drive expression of the therapeutic transgene encoding the FIX protein.

The AAV8 serotype was selected as the serotype for the insertion platform due to its ability to target the liver in multiple model systems, its favorable manufacturing profile, and its use as a liver-tropic vector in human clinical trials, including multiple trials in the hemophilia field.

The ALB locus is a liver safe harbor site that has robust expression in hepatocytes. In addition, there is the presence of a signal peptide in exon 1, which can be co-opted to mediate secretion of a transgene inserted downstream. The single guide RNA (sgRNA) directing the CRISPR/Cas9 component was designed to create a cut within intron 1 of the ALB gene. The inserted F9 gene relies on the ALB promoter and start codon for expression and relies on the ALB signal peptide for secretion.

The rAAV8-delivered DNA insertion template does not have any homology to the nucleotides flanking the ALB sgRNA target site and is inserted via homology-independent mechanisms. Therefore, the insertion cassette was designed to be bidirectional and encodes a copy of wild type FIX protein in both the sense and antisense orientation, such that either insertion orientation can support FIX protein expression. However, a unidirectional insertion cassette can also be used. A splice acceptor site is encoded upstream of each F9 transgene, and a polyadenylation sequence is encoded downstream of each copy of F9 (FIG. 2). The ALB exon 1 splice donor is thus joined with the splice acceptor encoded in the F9 DNA template.

In summary, the combination of the highly precise and targeted CRISPR/Cas9 technology delivered by LNP and the F9 DNA template delivered by the selected rAAV8 vector allows for long-term expression of wild type FIX protein from hepatocytes, potentially providing a life-long effective treatment to hemophilia B patients. In addition, the FIX expression can be tuned by efficiency of gRNA, the dose of AAV template, and the dose of LNP.

### Single Guide RNA Design and Selection

The ALB locus was selected as the insertion site for the F9 DNA template because it is the most highly expressed locus in hepatocytes, and because the signal peptide which mediates secretion of albumin is found in exon 1 and can be utilized to promote secretion of the inserted transgene. The human ALB locus was evaluated for the presence of the specific CRISPR/Cas9 protospacer adjacent motifs (PAM) sites to generate a list of target sites in the first intron of the locus where a Cas9-induced DNA cut could occur. The sites were cross-referenced against a single nucleotide polymorphism (SNP) analysis to ensure selected sites did not have common SNPs that might impact gRNA efficiency. A list of sgRNAs was then generated. See Table 10. Candidate sgRNAs were synthesized and formulated into LNPs with Cas9 mRNA for evaluation in vitro and in vivo.

TABLE 10
Human ALB Intron 1 Guide RNAs.

| | SEQ ID | SEQ | SEQ ID | SEQ ID NO |
|---|---|---|---|---|
| | NO (DNA- | ID NO | NO | (Guide RNA |
| Guide | Targeting | (Unmodified | (Modified | Target |
| RNA | Segment) | sgRNA) | sgRNA) | Sequence) |
| G009844 | 30 | 62 | 94 | 126 |
| G009851 | 31 | 63 | 95 | 127 |
| G009852 | 32 | 64 | 96 | 128 |
| G009857 | 33 | 65 | 97 | 129 |
| G009858 | 34 | 66 | 98 | 130 |
| G009859 | 35 | 67 | 99 | 131 |
| G009860 | 36 | 68 | 100 | 132 |
| G009861 | 37 | 69 | 101 | 133 |
| G009866 | 38 | 70 | 102 | 134 |
| G009867 | 39 | 71 | 103 | 135 |
| G009868 | 40 | 72 | 104 | 136 |
| G009874 | 41 | 73 | 105 | 137 |
| G012747 | 42 | 74 | 106 | 138 |
| G012748 | 43 | 75 | 107 | 139 |
| G012749 | 44 | 76 | 108 | 140 |
| G012750 | 45 | 77 | 109 | 141 |
| G012751 | 46 | 78 | 110 | 142 |
| G012752 | 47 | 79 | ill | 143 |
| G012753 | 48 | 80 | 112 | 144 |
| G012754 | 49 | 81 | 113 | 145 |
| G012755 | 50 | 82 | 114 | 146 |
| G012756 | 51 | 83 | 115 | 147 |
| G012757 | 52 | 84 | 116 | 148 |
| G012758 | 53 | 85 | 117 | 149 |
| G012759 | 54 | 86 | 118 | 150 |
| G012760 | 55 | 87 | 119 | 151 |
| G012761 | 56 | 88 | 120 | 152 |
| G012762 | 57 | 89 | 121 | 153 |
| G012763 | 58 | 90 | 122 | 154 |
| G012764 | 59 | 91 | 123 | 155 |
| G012765 | 60 | 92 | 124 | 156 |
| G012766 | 61 | 93 | 125 | 157 |

LNPs were first screened in primary human hepatocytes (PHH) using a bidirectional nanoluc-encoding AAV insertion template as a reporter. LNPs that supported targeted insertion of nanoluc were identified by measuring nanoluc protein secreted into the supernatant of PHH cultures.

Candidates that passed initial PHH screening were then tested for their ability to support in vivo gene insertion in genetically manipulated mice harboring both a humanized ALB locus and F9 gene deficiency. Top candidates from in vivo studies were functionally evaluated for off-target cutting.

LNPF9, which is formulated with ALB-targeting sgRNA 9860, was selected as the development candidate based on supporting robust hFIX expression levels across multiple platforms (primary human and non-human primate hepatocytes, ALB humanized mice, and non-human primates), lack of confirmed off-target sites, translation across species, lack of common human SNPs in the target site, low variability of hFIX expression within groups, and performance across a dose range (FIG. 3). The target site of sgRNA 9860 is conserved in cynomolgus monkeys. LNPF9 had no detectable off-target sites in the human genome (targeted amplicon sequencing (rhAMP-Seq) performed in two lots of primary human hepatocytes at saturating levels of editing failed to validate any locus other than on-target at ALB) and supported FIX expression via insertion in primary human and non-human primate hepatocytes, ALB humanized mice, and non-human primates.

### LNPF9 Description and Manufacture

LNPF9 is a lipid nanoparticle that includes a sgRNA of about 100 nucleotides in length and Cas9-encoding mRNA encapsulated in an LNP comprised of four different lipids. The composition of the LNP is summarized in Table 11. LNPF9 comprises four lipids at the following molar ratios: 50 mol % Lipid A, 9 mol % DSPC, 38 mol % cholesterol, and 3 mol % PEG2k-DMG and is formulated in aqueous buffer composed of 50 mM Tris-HCl, 45 mM NaCl, 5% (w/v) sucrose, at pH 7.4. The N:P ratio is about 6, and the gRNA:Cas9 mRNA ratio is about 1:2 by weight. The LNP is intended for single intravenous (IV) administration.

TABLE 11
Lipid Nanoparticle (LNPF9) Composition.

| Component | Description |
|---|---|
| Active | Cas9 mRNA |
| Pharmaceutical | sgRNA (gRNA9860) |
| Components | |
| Lipid Excipients | Lipid A: (9Z,12Z)-3-((4,4- |
| | bis(octyloxy)butanoyl)oxy)-2-((((3- |
| | (diethylamino)propoxy)carbonyl)oxy)methyl)propyl |
| | octadeca-9,12-dienoate |
| | Cholesterol |
| | DSPC |
| | PEG2K-DMG |
| Other Excipients | Tris, NaCl, Sucrose |
| | WFI |

LNPF9 consists of two CRISPR/Cas9 molecules, encapsulated in a lipid nanoparticle. First is sgRNA 9860, a single guide RNA (sgRNA) sequence consisting of 100 nucleotides with a molecular mass of 33 kilodaltons (kDa), and containing a 20 nucleotide sequence that is complementary to the target region in intron 1 of the human ALB gene. The target sequence recognized by g9860 is conserved in the cynomolgus monkey mfAlb gene intron 1, Second is a codon-optimized messenger RNA (mRNA) sequence encoding CRISPR-associated Cas9 protein, consisting of approximately 4,400 nucleotides with a molecular mass of approximately 1.5 megadaltons (MDa). The Cas9 protein, expressed from the Cas9 mRNA, is directed to cleave the DNA only if sgRNA 9860 binds to the targeted complementary DNA sequence associated with a PAM.

The ALB locus was selected as the targeted site for insertion since it is expressed specifically and robustly in liver hepatocytes. In addition, the ALB signal peptide is encoded in exon 1, which can be co-opted to mediate secretion of a transgene inserted downstream. The inserted F9 gene will thus rely on the ALB promoter and start codon for expression, and ALB signal peptide for secretion. Gene insertion within the ALB locus is not expected to materially affect global serum albumin levels since only a small fraction of ALB genes are modified through insertion.

Single guide RNA. The single guide RNA (sgRNA 9860) used in LNPF9 is a 100-mer oligonucleotide made through standard solid phase phosphoroamidite oligonucleotide synthesis. Manufacture consists of a 99-cycle chemical synthesis building off the 3′ terminal uracil nucleotide anchored to a solid support. Chemical modifications are incorporated into the 100-mer during synthesis, which include phosphorothioate (PS) linkages at the 5′- and 3′-end of the sgRNA and 2′-O-methyl modifications to some of the sugars of the RNA. After synthesis, the 100-mer is cleaved from the solid support followed by removal of the protecting groups. Purification of the crude 100-mer product is completed by ion exchange chromatography. The chromatographic fractions are pooled, buffer exchanged, desalted, and stored frozen at <−65° C. in water.

Cas9 mRNA. The Cas9 messenger RNA (mRNA) used in LNPF9 is based on the Cas9 protein sequence from Streptococcus pyogenes. The Cas9-encoding mRNA (SEQ ID NO: 225, with CDS set forth in SEQ ID NO: 226) is approximately 4400 nucleotides in length and is synthesized through an in vitro transcription (IVT) reaction using a linearized DNA template and T7 RNA polymerase. The sequence contains a 5′ cap, a 5′ untranslated region (UTR), an open reading frame (ORF) encoding the Cas9 protein, a 3′ UTR and a polyA tail. The 5′ cap is generated co-transcriptionally by use of a synthetic cap analogue structure, known as anti-reverse cap analogue (ARCA). The nucleotides (NTPs) used in the IVT reaction include three unmodified NTPs, ATP, CTP, and GTP, and one modified nucleotide, N1-methyl pseudo-UTP. After completion of the IVT reaction, crude mRNA is purified by selective precipitation with lithium chloride and tangential flow filtration. The final purified mRNA is stored frozen at −80° C. in water (≤−65° C.).

The uracils in the mRNA sequence have been completely replaced by a modified N¹ methylpseudouridine during the in vitro transcription. N¹ methylpseudouridine is a natural derivative of uracil and its incorporation into mRNA has been shown to increase protein expression. The 5′ end of the mRNA has a synthetic cap analog structure to increase stability. A poly-A tail of approximately 100 nucleotides confers additional intracellular stability to the mRNA molecules.

The Cas9 mRNA can be produced through in vitro transcription using recombinant enzymatic processes. Key components are summarized in Table 12. The template for mRNA synthesis can be the Cas9 encoding sequence contained within a plasmid produced in E. coli.

TABLE 12
Summary of Key mRNA Process Components.

| Component | Description | Purpose |
|---|---|---|
| Nucleotides | N1-Methylpseudouridine- 5′-triphosphate | Starting material |
| | Adenosine triphosphate | |
| | Cytidine triphosphate | |
| | Guanosine triphosphate | |
| mRNA Cap | ARCA or CleanCap ™ | Synthetic “cap” for mRNA |
| Proteins/Enzymes | T7 Polymerase | Recombinant enzyme for polymer extension |
| | | of mRNA |
| | RNase inhibitor | Recombinant enzyme to prevent breakdown of |
| | | mRNA by trace RNase present |
| | Pyrophosphatase | Recombinant enzyme to remove diphosphate |
| | | cleavage product (that inhibits T7pol) |
| | DNase (Turbo, TF) | Digests plasmid template after synthesis of |
| | | mRNA is complete |
| Plasmid encoding | Linearized plasmid | Template to encode |
| Cas9 gene sequence | | Cas9 mRNA |

The plasmid is extracted and purified by standard methods. This starting material is tested and released against defined specifications. The plasmid is linearized by restriction digest and purified away from this endonuclease before use as a template for making mRNA. The plasmid is stored frozen as a bulk solution.

Cas9 mRNA is produced by an in vitro transcription (IVT) reaction driven by a T7 RNA polymerase promoter provided by a linearized plasmid template. The IVT process proceeds through a 2-4 hour, 37° C. reaction where the T7 polymerase initiates transcription with a synthetic anti-reverse cap analog (e.g., ARCA) at the 5′ end of the nascent chain and then sequentially and progressively adds the appropriate corresponding nucleotides to the growing single stranded mRNA until reaching the end of the dsDNA template and releasing mRNA molecules of approximately 4,514 nucleotides.

Following the IVT reaction, the remaining plasmid DNA (pDNA) template is degraded by a DNase digestion. Subsequent downstream purification steps are performed to remove starting materials and concentrate the mRNA drug substance. The bulk Cas9 mRNA is subjected to release testing and stored frozen at or below −20° C.

Lipid excipients. The three non-proprietary lipids, 1,2-distearoyl-sn-glycero-3-phosphoethanolamine-N-[methoxy(polyethylene glycol)-2000] (PEG2K-DMG), 1,2-distearoyl-sn-glycero-3-phosphocholine (DSPC), and cholesterol can be manufactured to cGMP standards.

LNPF9 final formulation. A cross-flow mixing process encapsulates the sgRNA and Cas9 mRNA in LNPs. Lyophilized sgRNA is reconstituted in water for injection (WFI) and Cas9 mRNA solution is thawed. The sgRNA and mRNA are mixed. The controlled mixing process blended an aqueous buffered solution containing specified weight-based amounts of sgRNA and Cas9 mRNA with an ethanol solution containing the four lipid components at specific molar ratios. Following mixing, there is an inline dilution with WFI or buffer. The subsequent downstream processing involves ultrafiltration and diafiltration for final concentration and buffer exchange. The formulation is then sterile filtered using redundant sterilizing filters and filled into vials. The final product is a frozen LNP suspension containing sgRNA and Cas9 mRNA.

### DNA Template Design and Selection

The F9 DNA template delivered by rAAV8 vector was designed as a bidirectional cassette encoding the wild type FIX protein to support strong on-target FIX expression while minimizing immunogenicity. However, a unidirectional cassette can also be used. The F9 DNA template is a promoterless template, thus relying on the targeted ALB locus promoter for expression.

The AAV inverted terminal repeats (ITRs) flanking the cassette were derived from AAV2. A partial wild type F9 coding sequence (CDS) is encoded in both the sense and the antisense orientation within the DNA template. These F9 transgenes lack the native FIX signal peptide and rely on the ALB exon 1 signal peptide instead, but the F9 transgenes retain a partial FIX propeptide sequence. Each copy of F9 uses a unique coding sequence; one is a modified version of the consensus human CDS, and the other was codon optimized. The two copies of F9 are designed to be as divergent in sequence from one another as possible to limit intramolecular hybridization between the two copies and the formation of hairpin structures in the plasmid or AAV DNA. Both copies are depleted of CpG dinucleotide motifs, and cryptic splice donor motifs were removed where appropriate.

The splice acceptor sequences at the 5′ end of each F9 transgene were derived from mouse Alb exon 2 splice acceptor. The polyadenylation sequences at the 3′ end of the F9 transgenes were derived from simian virus 40 (SV40) and bovine growth hormone (bGH).

To select a development candidate, several versions of the bidirectional insertion cassette were generated in which the nucleotide sequence encoding one or both copies of F9 was modified. Table 13 lists the different versions of F9 inserts tested, and Table 14 lists the different bidirectional insertion cassettes tested. Candidate insertion templates were compared in vitro, in primary human hepatocytes (PHH), primary cynomolgus monkey hepatocytes (PCH), and in vivo in mice, and were evaluated for FIX protein expression and variability. A lead AAV template was selected based on supporting robust hFIX expression levels across multiple platforms (primary human and non-human primate hepatocytes, ALB humanized mice, and non-human primates), reduction of cryptic splicing events, minimal CpG content, and manufacturability (packaging efficiency/yields, empty/full ratios). The “native CpG removed no splice” F9 insert in Table 13 refers to a native F9 sequence in which 18 CpG dinucleotides were removed and 3 cryptic splice donor sites were removed (unavoidable introducing one novel CpG dinucleotide motif). The “codon optimized” F9 insert in Table 13 and that was used in Insert 72 has 74 CpGs, and the “native” F9 insert used in Insert 72 has 18 CpGs. Insert 27, Insert 28, Insert 29, and Insert 30 in Table 14 all contain only a single CpG within the ITRs.

TABLE 13
F9 Inserts for Insertion Cassettes.

| F9 Insert | SEQ ID NO |
|---|---|
| Native F9 Insert | 158 |
| Native CpG removed no splice F9 Insert | 159 |
| Native CpG removed F9 Insert | 160 |
| Native splice removed F9 Insert | 161 |
| Codon optimized F9 Insert | 162 |
| COMP F9 Insert | 163 |
| DC F9 Insert | 164 |
| GA F9 Insert | 165 |
| CpG0 F9 Insert | 166 |
| CpG3 F9 Insert | 167 |
| CpG 10 no CpGF9 Insert | 168 |
| CpG10 F9 Insert | 169 |
| CpG20 no CpG F9 Insert | 170 |
| CpG20 F9 Insert | 171 |

TABLE 14
Bidirectional Insertion Cassettes.

| | | | SEQ ID NO |
|---|---|---|---|
| | | | (with ITRs; |
| Con- | | | without |
| struct | F9 #l | F9 #2 | ITRs) |
| 72 | Native | Codon optimized | 172; 202 |
| 18 | Native | COMP | 173; 203 |
| 19 | Native | DC | 174; 204 |
| 20 | Native | GA | 175; 205 |
| 21 | Native | CpG0 | 176; 206 |
| 27 | Native CpG removed no splice | COMP | 177; 207 |
| 28 | Native CpG removed no splice | DC | 178; 208 |
| 29 | Native CpG removed no splice | GA | 179; 209 |
| 30 | Native CpG removed no splice | CpG0 | 180; 210 |
| 36 | Native CpG removed no splice | Codon optimized | 181; 211 |
| 37 | Native CpG removed | Codon optimized | 182; 212 |
| 38 | Native | Codon optimized | 183; 213 |
| 39 | Native splice removed | Codon optimized | 184; 214 |
| 22 | Native | CpG3 | 185; 215 |
| 23 | Native | CpG10 no CpG | 186; 216 |
| 24 | Native | CpG01 | 187; 217 |
| 25 | Native | CpG20 no CpG | 188; 218 |
| 26 | Native | CpG20 | 189; 219 |
| 31 | Native CpG removed no splice | CpG3 | 190; 220 |
| 32 | Native CpG removed no splice | CpG01 no CpG | 191; 221 |
| 33 | Native CpG removed no splice | CpG01 | 192; 222 |
| 34 | Native CpG removed no splice | CpG20 no CpG | 193; 223 |
| 35 | Native CpG removed no splice | CpG20 | 194; 224 |

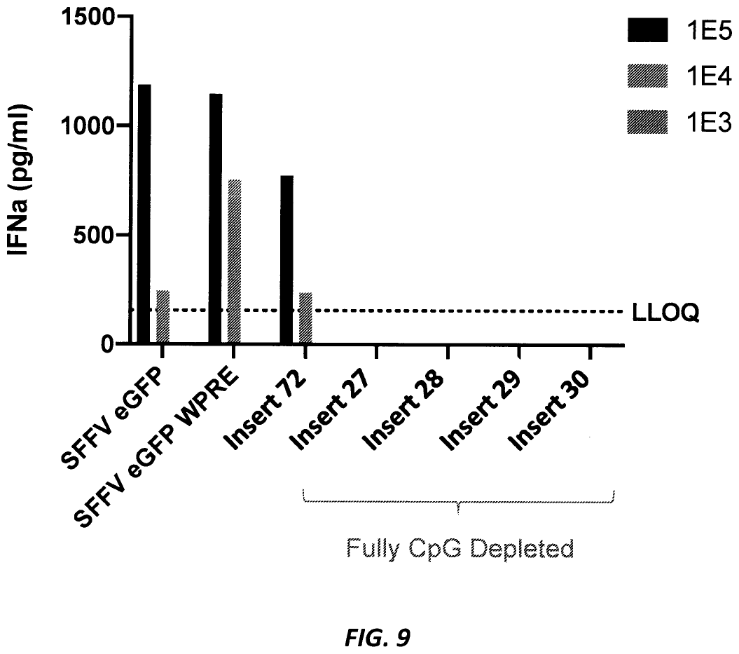

The selected development candidate is a bidirectional insertion cassette encoded by Insert 30, which, when packaged into rAAV8, is hereby denoted as REGV131 (FIG. 3). REGV131 has demonstrated FIX protein expression via insertion in primary human and non-human primate hepatocytes, ALB humanized mice, and non-human primates. Compared to other bidirectional F9 insertion cassettes (e.g., Insert 72), Insert 30 maintains hFIX expression (levels, activity, low variability, manufacturability) while also removing potential negative features that might impact template performance in human patients, minimizing unintended cryptic splicing events observed in wild type mice and non-human primates, and depleting CpGs because CpG sites in AAV viral vectors contain unmethylated C's, which can be potent TLR9 agonists. To demonstrate this, peripheral blood mononuclear cells (PBMCs) were isolated from human blood. Plasmacytoid dendritic cells (pDCs) were enriched and combined with pBMCs (1e4 pDCs+1e5 PBMCs per well). The cells were incubated for 16-18 hours with AAV or control CpG-oligodeoxynucleotides (ODNs). The supernatants were harvested, and an IFNα ELISA was performed. CpG-depleted F9 sequences (e.g., Insert 29 and Insert 30) elicited reduced IFN-I responses in a primary human plasmacytoid DC-based assay as compared to non-CpG-depleted F9 sequences (FIG. 9).

REGV131 (rAAV8 Vector) Description and Manufacture

REGV131 consists of a bidirectional F9 rAAV ssDNA genome cassette, encoding wild type FIX protein, packaged into a replication-incompetent rAAV8 capsid. Wild type FIX was chosen based on robust expression in preclinical studies, which obviated the need for the use of a hyperactive variant FIX. AAV8 was selected as the serotype for the insertion platform due to its ability to target the liver in multiple model systems, its favorable manufacturing profile, and its use as a liver-tropic vector in human clinical trials.

The DNA template leverages AAV2-derived ITRs at each end, and comprises two F9 coding sequences (CDSs), with each CDS flanked by a splice acceptor and polyadenylation (polyA) sequence.

The F9 DNA template does not have any homology to the nucleotides flanking the ALB sgRNA target site and is inserted via a homology-independent mechanism. Therefore, the insertion cassette is designed to be bidirectional and encodes two tandem inverted F9 transgenes, such that either insertion orientation can support FIX protein expression. These F9 transgenes lack the native FIX signal peptide, since they will rely on the ALB exon 1 signal peptide for secretion, but retain a partial FIX propeptide sequence for intracellular proteolytic processing.

Each copy of F9 uses a unique coding sequence; one is a modified version of the consensus human CDS and the other was codon optimized. The two copies of F9 are designed to be as divergent in sequence from one another as possible to limit intramolecular hybridization between the two copies and the formation of hairpin structures in the plasmid or AAV DNA. Both copies are depleted of CpG dinucleotide motifs to minimize transgene expression-limiting immune responses, and cryptic splice donor motifs have been removed where appropriate.

The splice acceptor sequences at the 5′ end of each F9 transgene are derived from mouse Alb exon 2 splice acceptor. The polyadenylation sequences at the 3′ end of the F9 transgenes are derived from Simian Virus 40 (SV40) and bovine growth hormone (bGH).

REGV131 injection is a sterile injectable product of purified rAAV8 vector containing the selected DNA template formulated as an aqueous buffered solution at predetermined strengths. The drug substance (DS) is manufactured utilizing triple transfection of HEK293 cells followed by further manufacturing into a sterile injectable drug product.

Structure of REGV131. REGV131 is an AAV-based vector derived from AAV serotype 8. The REGV131 genome is a single-stranded deoxyribonucleic acid (DNA), comprising inverted terminal repeats (ITR) at each end. The ITRs flank two different codon-optimized, CpG minimized, coding DNA (cDNA) sequences of a bidirectional, promoterless insertion template of the wild type FIX protein, as illustrated in FIG. 3. REGV131 is a non-replicating vector that will deliver the DNA bidirectional template to the targeted cells.

As demonstrated in the following examples, the bidirectional template integrates in the target cell genome within ALB intron 1 following homology-independent DNA repair at the CRISPR/Cas9 (LNPF9) target site. As demonstrated in the following examples, this integration results in the stable expression of a fusion protein consisting of the FIX propeptide and the residual amino acids from albumin exon 1, namely a signal sequence and 8 amino acids. Proteolytic cleavage occurs during secretion, and results in the release of the wild type FIX mature protein into the plasma or extracellular compartment.

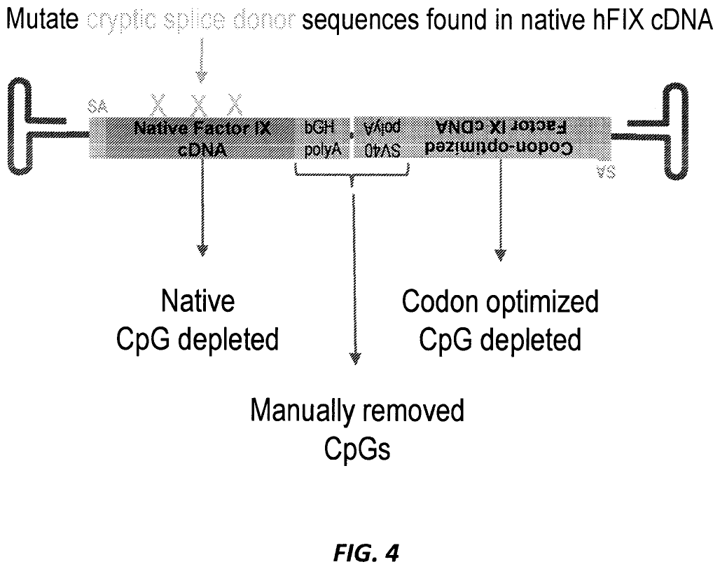

The bidirectional template plasmid, depicted in FIG. 4, contains two coding sequences for the F9 gene, each separately CpG minimized and codon-optimized in order to minimize complementarity. This design ensures the template will lead to functional FIX expression regardless of the insert direction. Placed after the stop codon of each F9 gene, in the middle of the construct, is one of two polyA sequences. One is originally taken from the bovine growth hormone gene (bGH polyA), and the other from a sequence derived from the simian virus 40 polyA (SV40 polyA), both of which are commonly used in transgene constructs designed for several mammalian hosts and known to be efficient polyA signals. Each F9 gene in the construct is promoterless, and instead a 3′ splice acceptor site is encoded to allow the albumin signal sequence to be fused to transgene product after transcription from the native ALB locus.

The plasmid sequence encoding the AAV virus genome (vg) was inserted into a plasmid backbone containing a bacterial origin of replication (ori) and a bacterial expression cassette encoding the kanamycin resistance gene (KanR).

A rep/cap plasmid Rep2/Cap8 is one of the two plasmids encoding viral proteins that are co-transfected into cells with the bidirectional template plasmid to enable proper assembly of the AAV, and its intended F9 transgenes. Replication/cap plasmids encode for the two main components of the wild type AAV genome: the rep gene and the cap gene that are necessary for assembly of the protein capsid, processing the DNA encoding the AAV transgenic genome into single-stranded DNA (ssDNA), and packaging ssDNA viral genome into the capsid. The plasmid contains a chimeric sequence using the rep gene from AAV2 and the cap gene from AAV8. Because only the cap gene encodes the proteins in the protein capsid, this is sufficient to make recombinant AAV with an AAV8 serotype. The rep transcripts are under the control of a truncated version of the wild-type AAV P5 promoter, while cap transcripts are under control of the AAV p40 promoter, which requires additional adenoviral proteins to drive transcription and translation. The P5-rep/cap system was synthesized into a standard plasmid backbone containing bacterial ori and KanR.

The plasmid pAd-Helper-Kan is a helper plasmid that encodes the necessary adenoviral proteins for the AAV producer cells to successfully express Rep and Cap proteins from the AAV rep/cap plasmid. The adenoviral proteins drive the needed DNA replication and packaging of the AAV transgene from the transfer plasmids, and perform additional functions needed to drive AAV production. The series of adenoviral open reading frames (ORFs) and RNA encoded in pAd-Helper-Kan are sufficient to drive this task without requiring use of full-length adenoviral genomes or infective adenovirus as a helper agent for AAV production.

REGV131 can be manufactured by triple transfection of the commercial HEK293 cell line CTS Viral Production Cells (Cat #A3152801, Thermo Fisher Scientific).

The REGV131 can be formulated, for example, in 10 mM sodium phosphate, 180 mM sodium chloride, and 0.005% poloxamer 188, at pH 7.3.

### REGV131-LNPF9 for Treatment of Hemophilia B

REGV131 and LNPF9 are planned to be administered systemically by intravenous infusion. Upon entry into hepatocytes, REGV131 delivers the F9 DNA template to the nucleus, while LNPF9 delivers the two RNA molecules to the cytoplasm. Once translated, Cas9 protein will complex with sgRNA 9860 to form a ribonucleoprotein (RNP) complex, which can translocate to the nucleus and cut the host dsDNA at the sgRNA 9860 target site in intron 1 of the ALB locus. The F9 DNA template can subsequently be inserted at the DSB site by NHEJ.

Following insertion of the F9 DNA template into ALB intron 1, mRNA transcription from the endogenous ALB promoter proceeds through ALB exon 1 and continues into the F9 DNA template until it reaches the polyA sequence, signaling termination of transcription. Following splicing between ALB exon 1 splice donor and the splice acceptor encoded in the F9 insertion template, the resulting mRNA is a fusion transcript between ALB exon 1 and F9 coding sequence. During translation, the ALB signal peptide encoded in exon 1 mediates entry of the polypeptide into the secretory pathway and is proteolytically removed during secretion. The FIX polypeptide is further processed intracellularly to remove the FIX propeptide, thus yielding mature and functional wildtype FIX protein, which is then secreted into the circulation and detected in plasma.

In summary, the combination of the highly precise and targeted CRISPR/Cas9 technology delivered by LNPF9 and the F9 DNA template delivered by the selected rAAV8 vector (REGV131) is expected to allow for expression of wildtype FIX protein from hepatocytes, potentially providing a life-long effective treatment to hemophilia B patients.

### Example 2. In Vitro Validation

### FIX Protein Expression Levels in Primary Human Hepatocytes

To confirm that targeted insertion of rAAV8-delivered DNA templates can occur in human hepatocytes with our lead rAAV candidates coupled with our lead LNP candidate, expression of wild type FIX protein from the ALB locus was evaluated in vitro in primary human hepatocytes (PHH).

The PHH were treated with rAAV8 packaged with either the F9 DNA template encoded by Insert 30 or two other candidate insertion templates (Insert 72 and Insert 29), with or without LNPF9 to target the insertion of the templates into ALB intron 1. See FIG. 2. FIX protein concentration was measured 8 days later using an ELISA assay for hFIX (Abcam #ab188393).

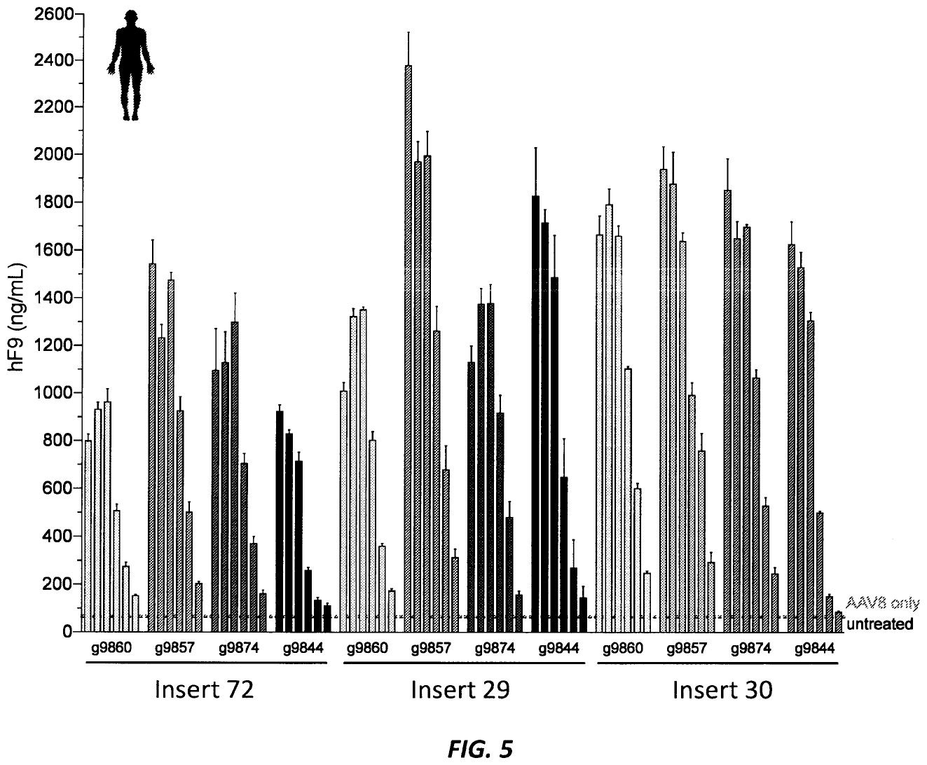

Cells left untreated expressed low levels of endogenous hFIX, and cells transduced with rAAV8-packaged F9 DNA templates alone expressed similar low endogenous levels of hFIX. In contrast, cells treated with rAAV8-delivered F9 DNA insertion templates and LNPF9 or LNPs containing other sgRNAs targeting human ALB intron 1 (e.g., g9857, g9874, and g9844) secreted hFIX in a LNP dose-dependent manner (FIG. 5).

In conclusion, targeted insertion of F9 DNA templates into the ALB locus of PHH resulted in FIX protein expression in vitro. The F9 DNA template produced from Insert 30 supported higher levels of hFIX than other DNA templates when tested head to head in PHH along with LNPF9.

### FIX Protein Expression Levels in Primary Non-Human Primate Hepatocytes

To confirm that targeted insertion of rAAV8-delivered DNA templates can occur in non-human primate hepatocytes with our lead rAAV8 candidates along with our lead LNP candidate, expression of wild type FIX protein from the ALB locus was evaluated in vitro in primary cynomolgus monkey hepatocytes (PCH).

The PCH were treated with rAAV8 packaged with either the AAV template encoded by Insert 30 or two other candidate insertion templates (Insert 72 and Insert 29), with or without LNPF9 to target the insertion of the templates into ALB intron 1. FIX protein concentration was measured 8 days later using an ELISA assay for hFIX (Abcam #ab188393).

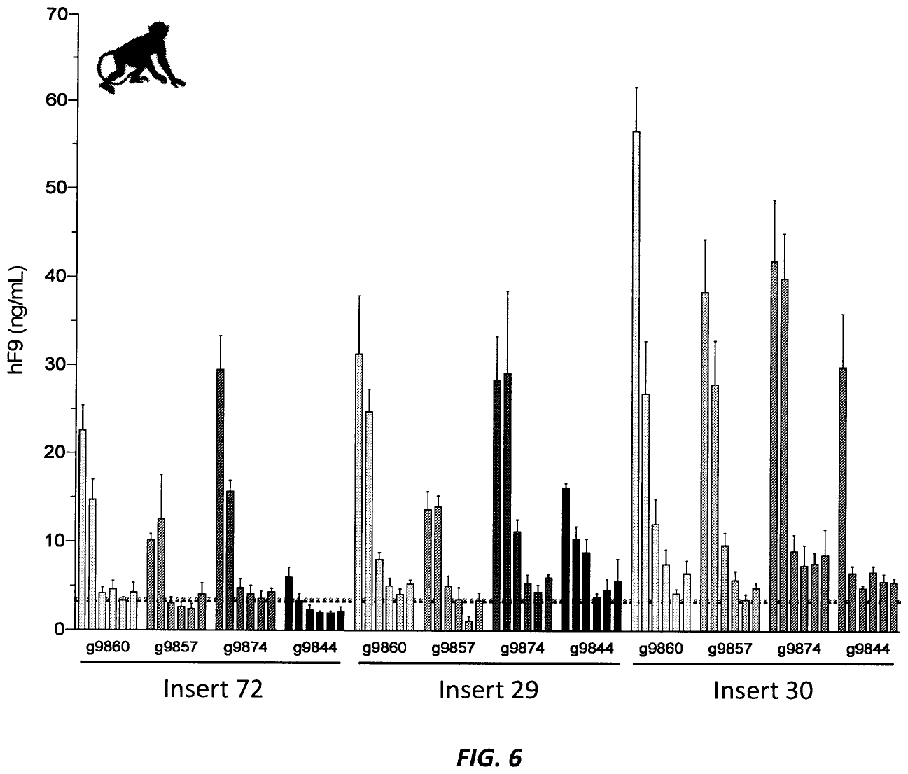

In PCH left untreated and cells transduced with rAAV8-packaged F9 DNA templates alone, low levels of background hFIX signal were measured. In contrast, cells treated with rAAV8-delivered F9 DNA insertion templates and LNPF9 or LNPs containing other sgRNAs targeting human ALB intron 1 (e.g., g9857, g9874, and g9844) secreted hFIX in a LNP dose-dependent manner (FIG. 6).

In conclusion, targeted insertion of F9 DNA templates into the ALB locus of PCH resulted in FIX protein expression in vitro. These results demonstrate that hepatocytes derived from non-human primates can support expression of hFIX via insertion using the LNPF9 and rAAV8-packaged F9 DNA template Insert 30 (REGV131), and further support the choice of non-human primates as a suitable large animal model for translational studies with these candidates.

### Example 3. In Vivo Human FIX Protein Expression and Activity in a Mouse Disease Model of Hemophilia B

LNPF9 (g9860) and REGV131 (Insert 30) were evaluated in a dose matrix study for levels of human wild type FIX protein (hFIX) expression and activity in genetically manipulated mice harboring the humanized ALB locus and lacking FIX activity attributed to a F9 gene deficiency, similar to hemophilia B patients who have either a deficiency or a dysfunction of FIX.

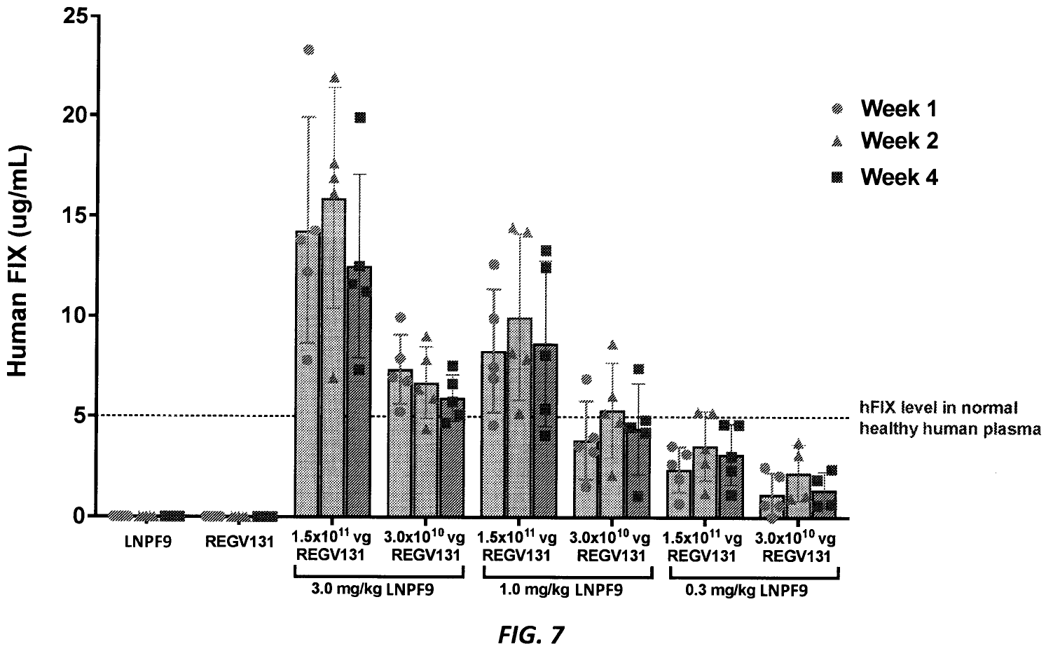

The ALB^{hu/hu}/F9^{-/-} mice were dosed intravenously with LNPF9 (0.3, 1.0, or 3.0 mg/kg) and/or REGV131 (3.0E10 or 1.5E11 vg/mouse). Groups treated with LNPF9 or REGV131 alone did not express hFIX in plasma over the 4 weeks of monitoring. However, animals treated with a combination of LNPF9 and REGV131 expressed plasma hFIX in a dose-dependent manner (FIG. 7).

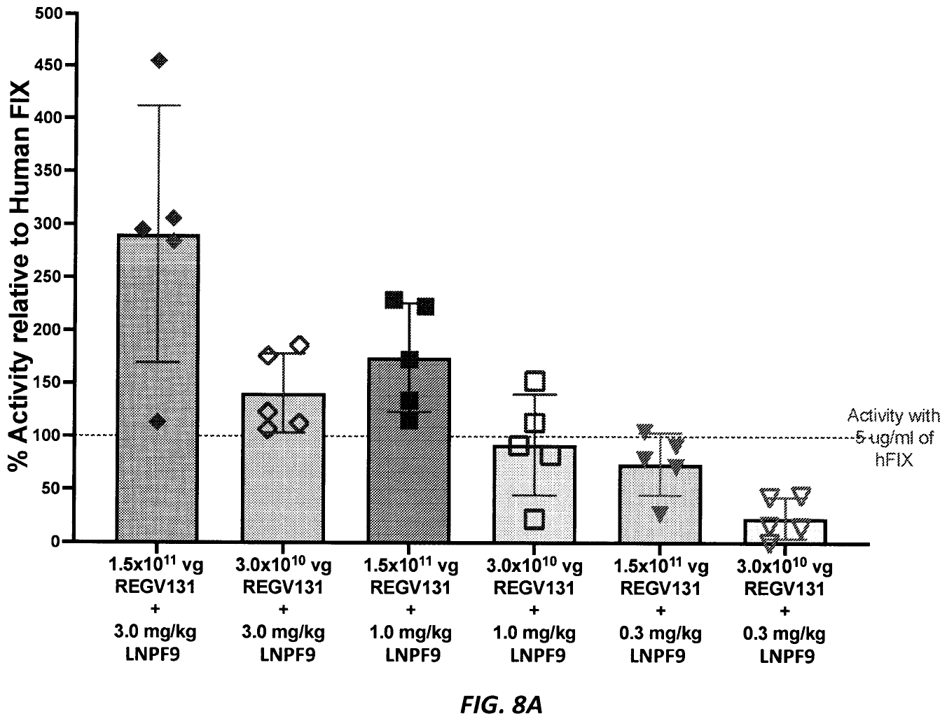

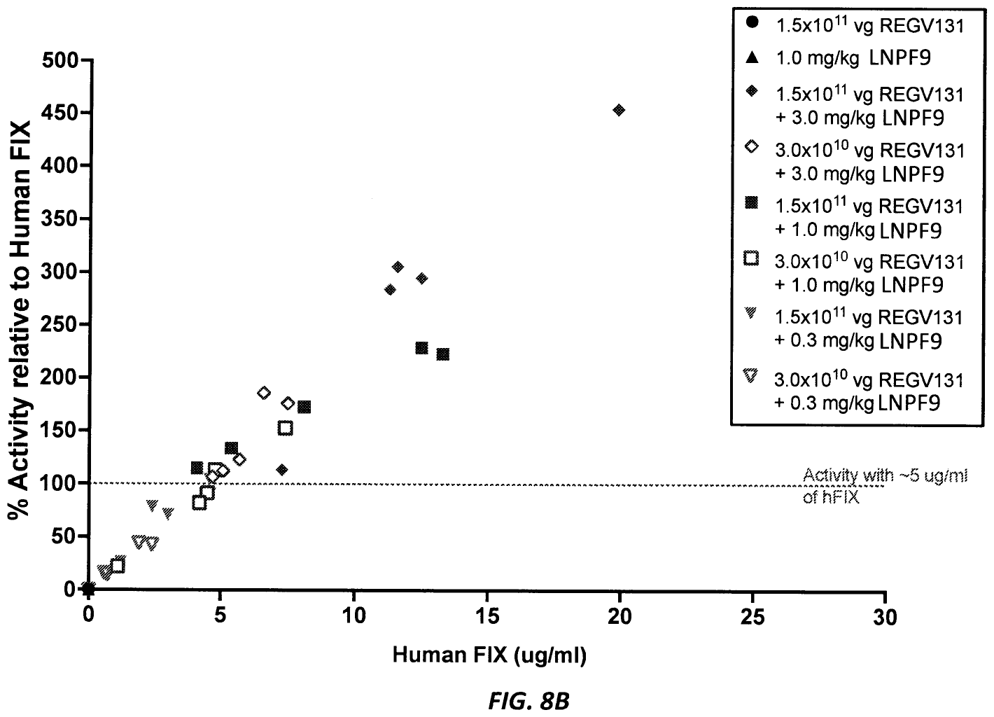

Plasma from ALB^{hu/hu}/F9^{-/-} mice treated with a combination of LNPF9 and REGV131 expressed hFIX that exhibited hFIX activity (FIG. 8A). Plasma was isolated from mice at 4 weeks post-treatment and mouse-generated hFIX was pulled-down using capture antibody specific for hFIX (AHIX-5041; Haematologic Technologies), then a chromogenic assay (BioPhen FIX, Hypen BioMed) was used to measure relative hFIX activity. hFIX levels correlated with hFIX activity at week 4 (FIG. 8B). Data are plotted for individual animals as a correlation of level-to-activity.

In summary, this proof-of-concept study demonstrated that ALB^{hu/hu}/F9^{-/-} mice with a FIX deficiency were able to express functional hFIX following CRISPR/Cas9 insertion of hF9 gene into the humanized ALB locus.

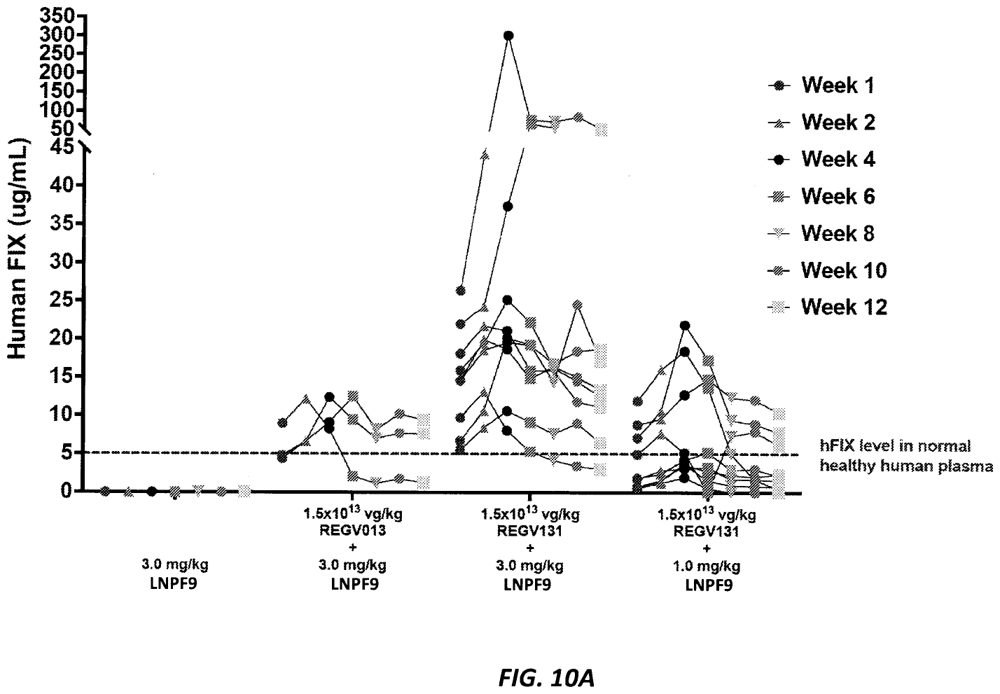

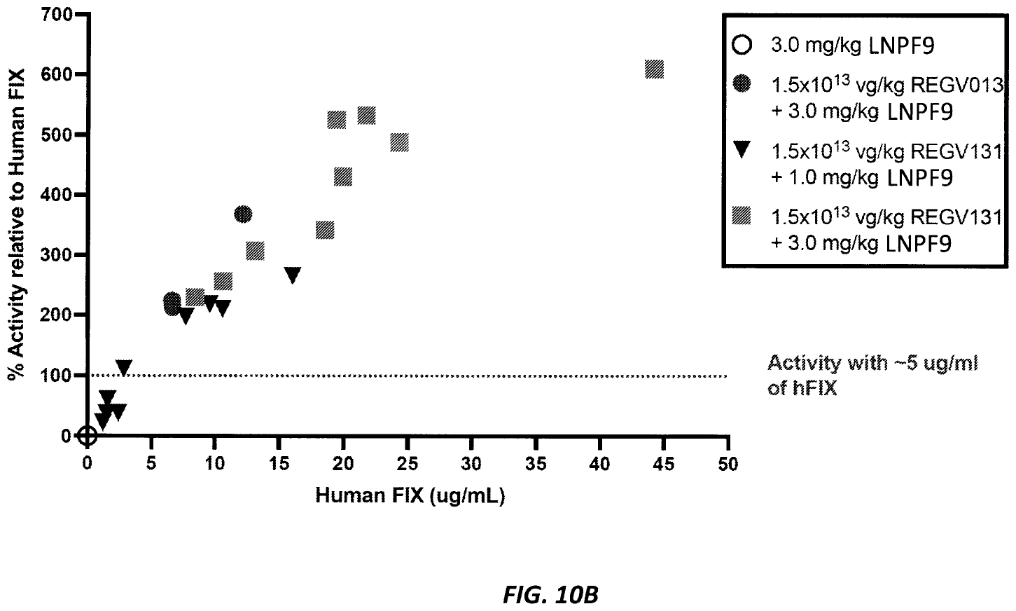

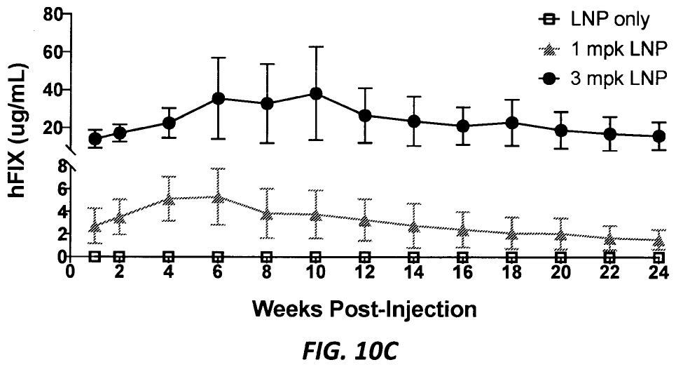

The combined data showed that the F9 DNA template in Insert 30 produced higher levels of hFIX expression than the first generation template (Insert 72) in PHH (see FIG. 5), in PCH (see FIG. 6), and in vivo in cynomolgus monkeys (see FIG. 10A). In addition, the Insert 30 template resulted to less IFNα production in response to the vector as compared to Insert 72 (FIG. 9).

### Example 4. In Vivo Human FIX Protein Expression and Activity in Non-Human Primates

The combination of LNPF9 (g9860) and REGV131 (Insert 30) was evaluated for efficiency of human FIX (hFIX) expression and activity in male and female Cynomolgus monkeys (non-human primates (NHP)) as compared to the combination of LNPF9 and REGV013 (Insert 72). The target site of g9860 is conserved in Cynomolgus monkeys, so the Insert 30 can be inserted into the monkey ALB locus.

The monkeys were dosed intravenously with LNPF9 (1.0 or 3.0 mg/kg) and/or REGV131 (1.5×10¹³ vg/kg) or REGV013 (1.5×10¹³ vg/kg). A control group treated with only 3.0 mg/kg LNP did not express hFIX in plasma during the 4-week post-injection monitoring period. However, monkeys treated with a combination of LNPF9 and REGV131 express plasma hFIX in an LNP-dose-dependent manner (FIG. 10A). Insert 30 produced higher FIX levels than Insert 72 when dosed at 1.5×10¹³ vg/kg together with 3.0 mg/kg of LNPF9. Even when administered together with a lower dose of LNPF9 (1.0 mg/kg), Insert 30 still produced at least as much human FIX as Insert 72 when administered with the higher dose of LNPF9 (3.0 mg/kg). In some monkeys dosed intravenously with LNPF9 (1.0 or 3.0 mg/kg) and/or REGV131 (1.5×10¹³ vg/kg), expression of hFIX in plasma was assessed for six-months (FIG. 10C). The treatment resulted in dose-responsive hFIX expression for the entire 6 months in the non-human primates.

Monkey-expressed hFIX proteins were isolated from monkey plasma, which have endogenous monkey FIX, using an antibody specific for human FIX (AHIX-5041; Haematologic Technologies) to assess hFIX function in an ex vivo FIX activity assay. Plasma from monkeys treated with only LNPF9 had no hFIX protein and thus demonstrated no hFIX activity. However, plasma from monkeys treated with a combination of LNPF9 and REGV131 expressed hFIX protein and demonstrated an LNP-dose-dependent response for hFIX activity as shown in the correlation analysis of hFIX expression to hFIX activity (FIG. 10B, Table 15).

TABLE 15
Correlation of hFIX Expression to hFIX Activity at Week 2.

| LNP and Virus Dose | Relative hF9 % Activity | hF9 μg/mL |
|---|---|---|
| g9860-3 mpk | 0 | N/D |
| PBS | | |
| Male | | |
| g9860-3 mpk | 213.2 | 6.65 |
| Insert 72-1.5E13 | 368.7 | 12.19 |
| Male | 224.4 | 6.63 |
| g9860-3 mpk | 526.0 | 19.44 |
| Insert 30-1.5E13 | 610.5 | 44.23 |
| Male | 256.4 | 10.64 |
| | 342.2 | 18.58 |
| | 488.6 | 24.31 |
| | 230.5 | 8.44 |
| g9860-3 mpk | 533.7 | 21.77 |
| Insert 30-1.5E13 | 431.6 | 19.98 |
| Female | 307.4 | 13.13 |
| g9860-1 mpk | 60.7 | 1.58 |
| Insert 30-1.5E13 | 197.6 | 7.69 |
| Male | 37.3 | 1.50 |
| | 265.7 | 16.05 |
| | 22.4 | 1.22 |
| | 38.5 | 2.42 |
| g9860-1 mpk | 218.3 | 9.59 |
| Insert 30-1.5E13 | 111.3 | 2.84 |
| Female | 210.4 | 10.60 |

In summary, this proof-of-concept study demonstrated that Cynomolgus monkeys were able to express functional hFIX following CRISPR/Cas9 insertion of hF9 gene into the monkey ALB locus. These results are encouraging for patients with hemophilia B since the same LNP and AAV components used in the monkey study would target the hF9 gene for insertion into the human ALB locus in liver hepatocytes.

### Example 5. Durable Human FIX Protein Expression After Neonatal Insertion in Mice

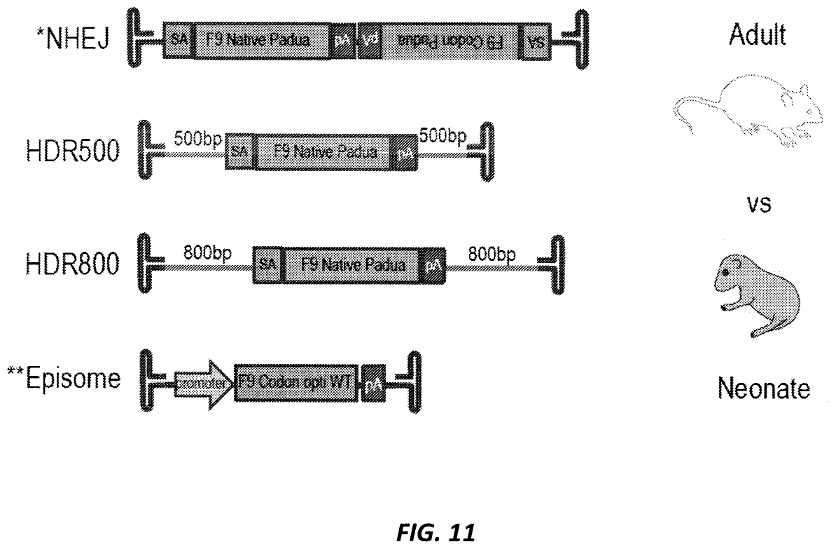

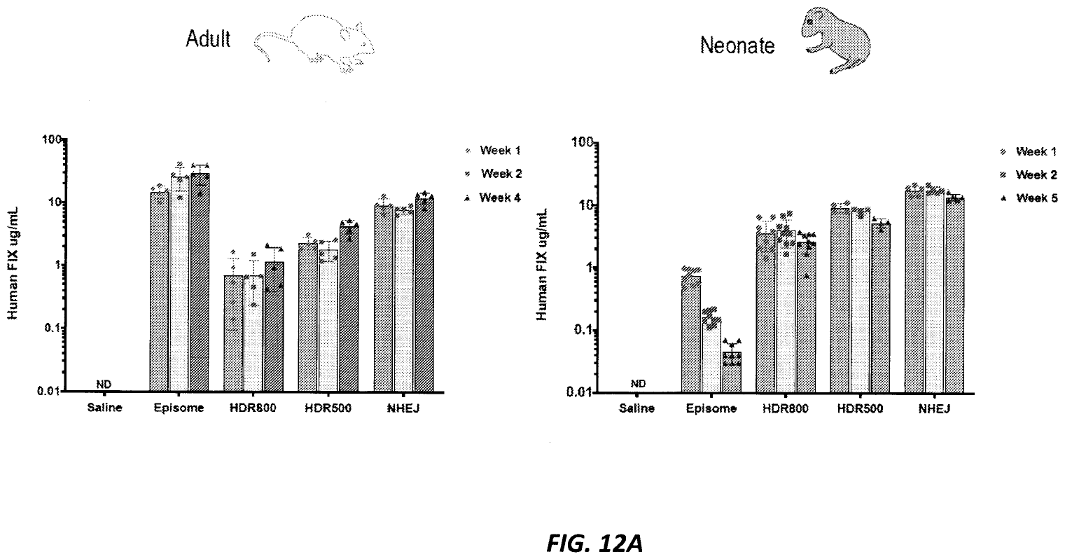

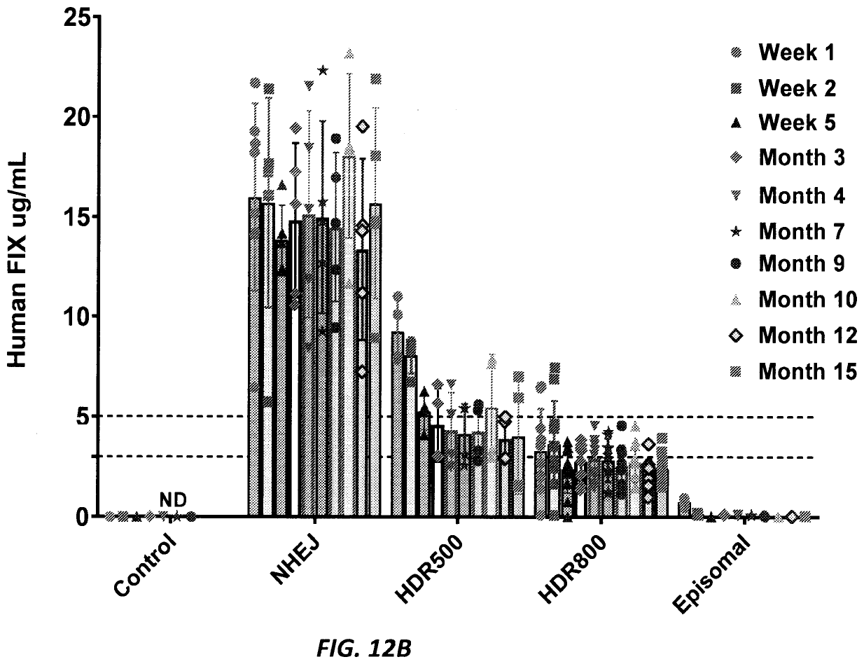

To compare episome-mediated expression versus insertion-mediated expression in adult and neonatal mice, and to compare different DNA repair pathways in adult and neonatal mice, we compared hFIX serum levels following administration of a hFIX episome (expression driven by hAAT promoter), a bidirectional hFIX NHEJ insertion template, a hFIX HDR insertion template with homology arms of 500 bp, and a hFIX HDR insertion template with homology arms of 800 bp. See FIG. 11. Neonatal C57BL/6 mice were dosed at P0 or P1 with the following: (1) 4 mg/kg of LNP-g666 and 3e9 vg/mouse of rAAV8 with the hFIX-HDR-500 template; (2) 4 mg/kg of LNP-g666 and 3e9 vg/mouse of rAAV8 with the hFIX-HDR-800 template; (3) 4 mg/kg of LNP-g666 and 3e9 vg/mouse of rAAV8 with the hFIX-NHEJ template; or (4) 3e9 vg/mouse of rAAV8 episomal template. Saline-injected mice were used as a negative control. The hFIX coding sequence in the episomal AAV was a codon-optimized sequence encoding wild type human F9. The hFIX coding sequence in the two HDR constructs was the native human F9 coding sequence with the Padua mutation (R338L). Blood was collected and plasma prepared at 1 week, 2 weeks, and 5 weeks post-dosing. hFIX levels were measured by human FIX ELISA. The experiment was then repeated in adult C57BL/6 mice, with the adult mice being dosed with the following: (1) 0.8 mg/kg of LNP-g666 and 2e10 vg/mouse of rAAV8 with the hFIX-HDR-500 template; (2) 0.8 mg/kg of LNP-g666 and 2e10 vg/mouse of rAAV8 with the hFIX-HDR-800 template; (3) 0.8 mg/kg of LNP-g666 and 2e10 vg/mouse of rAAV8 with the hFIX-NHEJ template; or (4) 2e10 vg/mouse of rAAV8 episomal template. Saline-injected mice were used as a negative control. Blood was collected and plasma prepared at 1 week, 2 weeks, and 4 weeks post-dosing. The results are shown in FIG. 2 and Tables 12-13. Episome-mediated expression was low even at the first time point compared to insertion-mediated expression in neonates and was lost over time in neonates. The opposite was observed in adult mice: episome-mediated expression was higher at the first time point and subsequent time points compared to insertion-mediated expression in adult mice. These results confirmed what was observed in a previous similar experiment (data not shown). In contrast to the results in the neonatal mice, hFIX levels stayed steady in adult mice with both episomal and insertion constructs, with the episomal construct giving the highest expression. See FIGS. 12A-12B and Tables 16-18.

TABLE 16
Human FIX Serum Levels (μg/mL) in Neonatal Mice.

| | Episome | | | | | | | | |
|---|---|---|---|---|---|---|---|---|---|
| W 1 | 0.9 | 0.56 | 0.48 | 0.52 | 0.96 | 0.67 | 0.93 | 0.98 | 0.77 |
| W 2 | 0.2 | 0.11 | 0.12 | 0.14 | 0.15 | 0.17 | 0.22 | 0.21 | 0.15 |
| W 5 | 0.06 | 0.03 | 0.03 | 0.07 | 0.07 | 0.03 | 0.04 | 0.04 | 0.04 |

| | NHEJ | | | | |
|---|---|---|---|---|---|
| W 1 | 14.22 | 19.28 | 14.12 | 18.67 | 21.69 |
| W 2 | 16.08 | 17.25 | 16.07 | 17.66 | 21.39 |
| W 5 | 12.29 | 12.41 | 14.18 | 16.61 | 13.71 |

HDR500

| | | | | |
|---|---|---|---|---|
| W 1 | 11.03 | 8.02 | 7.86 | 10.11 |
| W 2 | 8.29 | 6.74 | 8.63 | 8.61 |
| W 5 | 6.27 | 4.09 | 5.24 | 5.49 |

| | HDR800 | | | | | | | | | |
|---|---|---|---|---|---|---|---|---|---|---|
| W 1 | 3.86 | 6.44 | 6.52 | 4.43 | | 1.41 | 2 | 3.58 | 2.71 | 2.07 |
| W 2 | 3.54 | 6.9 | 7.46 | 4.44 | 4.64 | 1.65 | 2.45 | 3.55 | 2.83 | 2.5 |
| W 5 | 2.4 | 3.49 | 3.48 | 3.33 | 3.77 | 0.77 | 2.19 | 2.7 | 2.5 | 1.68 |

TABLE 17
Human FIX Serum Levels (μg/mL) in Adult Mice.

| | Episome | | | | |
|---|---|---|---|---|---|
| W1 | 16.69 | 15.57 | 18.97 | 14.17 | 9.82 |
| W2 | 25.39 | 23.07 | 41.53 | 27.53 | 12.07 |
| W4 | 39.97 | 26.61 | 39.63 | 28.5 | 14.28 |
| | NHEJ | | | | |
| W1 | 8.1 | 9.52 | 13.09 | 6.55 | 9.2 |
| W2 | 9.15 | 8.12 | 8.18 | 6.46 | 7.27 |
| W4 | 14.86 | 10.86 | 13.85 | 8.42 | 13 |
| | HDR500 | | | | |
| W1 | 2.23 | 2.5 | 1.89 | 3.14 | 1.85 |
| W2 | 1.2 | 2.62 | 1.55 | 2.41 | 1.34 |
| W4 | 3.83 | 5.39 | 4.89 | 4.62 | 2.75 |
| | HDR800 | | | | |
| W1 | 1.65 | 0.94 | 0.26 | 0.54 | 0.14 |
| W2 | 0.69 | 1.52 | 0.24 | 0.67 | 0.46 |
| W4 | 2.13 | 1.87 | 0.49 | 0.94 | 0.43 |

TABLE 18
Human FIX Serum Levels (μg/mL) in Neonatal Mice.

| | | Wk 1 | Wk 2 | Wk 5 | Month 3 | Month 4 | Month 7 | Month 9 | Month 10 | Month 12 | Month 15 |
|---|---|---|---|---|---|---|---|---|---|---|---|
| | | hF9 | hF9 | hF9 | hF9 | hF9 | hF9 | hF9 | hF9 | hF9 | hF9 |
| Mice # | Group # | ug/mL | ug/mL | ug/mL | ug/mL | ug/mL | ug/mL | ug/mL | ug/mL | ug/mL | ug/mL |
| 1 | Ctrl 1 | ND | ND | ND | ND | ND | ND | ND | ND | ND | ND |
| 2 | Ctrl 2 | ND | ND | ND | ND | ND | ND | ND | ND | ND | ND |
| 3 | Ctrl 3 | ND | ND | ND | ND | ND | ND | ND | ND | ND | ND |
| 4 | Ctrl 4 | ND | ND | ND | ND | ND | ND | ND | ND | ND | ND |
| 5 | Ctrl 5 | ND | ND | ND | ND | ND | ND | ND | ND | ND | ND |
| 6 | Ctrl 6 | ND | ND | ND | ND | ND | ND | ND | ND | ND | ND |
| 7 | Ctrl 7 | ND | ND | ND | ND | ND | ND | ND | ND | ND | ND |
| 8 | Ctrl 8 | ND | ND | ND | ND | ND | ND | ND | ND | ND | ND |
| 9 | HDR500-1 | 11.03 | 8.29 | 6.27 | 3.04 | 3.12 | 3.13 | 3.32 | 3.35 | 2.91 | 1.60 |
| 10 | HDR500-3 | 8.02 | 6.74 | 4.09 | 3.05 | 2.51 | 2.59 | 2.80 | 2.98 | 2.95 | 1.54 |
| 11 | HDR500-4 | 7.86 | 8.63 | 5.24 | 6.61 | 5.11 | 5.44 | 5.62 | 7.64 | 4.76 | 5.97 |
| 12 | HDR500-5 | 10.11 | 8.61 | 5.49 | 5.68 | 6.60 | 5.43 | 5.33 | 7.89 | 5.00 | 7.00 |
| 13 | Episomal 1 | 0.90 | 0.20 | 0.06 | 0.16 | 0.14 | 0.12 | 0.04 | 0.05 | 0.04 | 0.04 |
| 14 | Episomal 2 | 0.56 | 0.11 | 0.03 | 0.09 | 0.09 | 0.08 | 0.03 | 0.03 | 0.04 | 0.06 |
| 15 | Episomal 3 | 0.48 | 0.12 | 0.03 | 0.11 | 0.09 | 0.09 | 0.02 | 0.04 | 0.02 | 0.03 |
| 16 | Episomal 4 | 0.52 | 0.14 | 0.07 | 0.14 | 0.11 | 0.09 | 0.03 | 0.02 | 0.02 | 0.05 |
| 17 | Episomal 5 | 0.96 | 0.15 | 0.07 | NA (mouse died) | | | NA (mouse died) | | | |
| 18 | Episomal 6 | 0.67 | 0.17 | 0.03 | 0.13 | 0.11 | 0.11 | 0.03 | 0.04 | 0.00 | 0.04 |
| 19 | Episomal 7 | 0.93 | 0.22 | 0.04 | 0.13 | 0.09 | 0.09 | 0.09 | 0.05 | 0.04 | 0.03 |
| 20 | Episomal 8 | 0.98 | 0.21 | 0.04 | 0.14 | 0.10 | 0.11 | 0.06 | 0.03 | 0.04 | 0.05 |
| 21 | Episomal 9 | 0.77 | 0.15 | 0.04 | 0.14 | 0.15 | 0.12 | 0.05 | 0.03 | 0.05 | 0.07 |
| 22 | HDR800-1 | 3.86 | 3.54 | 2.40 | 2.08 | 2.26 | 2.91 | 1.66 | 2.09 | 1.98 | 1.77 |
| 23 | HDR800-2 | 6.44 | 6.90 | 3.49 | 3.47 | 3.36 | 3.49 | ND | 2.92 | 2.24 | 2.31 |
| 24 | HDR800-3 | 6.52 | 7.46 | 3.48 | 2.80 | 3.62 | 3.45 | 2.71 | 2.70 | 1.95 | 2.54 |
| 25 | HDR800-4 | 4.43 | 4.44 | 3.33 | 3.59 | 4.55 | 4.14 | 3.22 | 3.75 | 2.51 | 3.28 |
| 26 | HDR800-5 | NA | 4.64 | 3.77 | 3.47 | 3.82 | 4.26 | 4.57 | 4.56 | 3.65 | 3.95 |
| 27 | HDR800-6 | 1.41 | 1.65 | 0.77 | 1.54 | 1.48 | 1.19 | 3.35 | 1.50 | 1.47 | 1.60 |
| 28 | HDR800-7 | 2.00 | 2.45 | 2.19 | 3.32 | 3.11 | 2.26 | 2.37 | 2.75 | 2.63 | 2.29 |
| 29 | HDR800-8 | 3.58 | 3.55 | 2.70 | 2.16 | 2.09 | 2.36 | 1.58 | 2.04 | 1.59 | 2.08 |
| 30 | HDR800-9 | 0.09 | 0.10 | 0.03 | NA (mouse died) | | | NA (mouse died) | | | |
| 31 | HDR800-10 | 2.71 | 2.83 | 2.50 | 3.87 | 3.36 | 3.12 | 2.52 | 3.58 | 2.45 | 2.80 |
| 32 | HDR800-11 | 2.07 | 2.50 | 1.68 | 1.35 | 1.66 | 1.28 | 1.15 | 1.45 | 1.01 | 1.50 |
| 33 | NHEJ 1 | 6.45 | 5.75 | NA (mouse died) | | | | NA (mouse died) | | | |
| 34 | NHEJ 2 | 14.22 | 16.08 | 12.29 | 10.58 | 8.44 | 9.27 | 9.45 | 11.67 | 7.24 | 8.95 |
| 35 | NHEJ 3 | 19.28 | 17.25 | 12.41 | 15.67 | 18.44 | 15.77 | 14.70 | 18.38 | 14.58 | 14.80 |
| 36 | NHEJ 4 | 15.19 | NA (mouse died) | | | | | NA (Mouse died) | | | |
| 37 | NHEJ 5 | 14.12 | 16.07 | 14.18 | 17.28 | 15.39 | 14.79 | 16.99 | 18.47 | 14.32 | 14.68 |
| 38 | NHEJ 6 | 18.67 | 17.66 | 16.61 | 19.46 | 21.53 | 22.32 | 18.93 | 23.21 | 19.53 | 21.90 |
| 39 | NHEJ 7 | 18.23 | NA (mouse died) | | | | | NA (Mouse died) | | | |
| 40 | NHEJ 8 | 21.69 | 21.39 | 13.71 | 11.15 | 11.88 | 12.72 | 12.36 | 18.53 | 11.19 | 18.07 |

These experiments showed that expression of inserted F9 is durable in neonatal livers, indicating that insertion of F9 templates into the albumin locus can result in durable expression in neonatal subjects. These genome integration provided durable expression that was maintained throughout the experiment in neonatal mice.

### Example 6. rAAV8 Viruses Produced Using Various ITR Length Plasmids

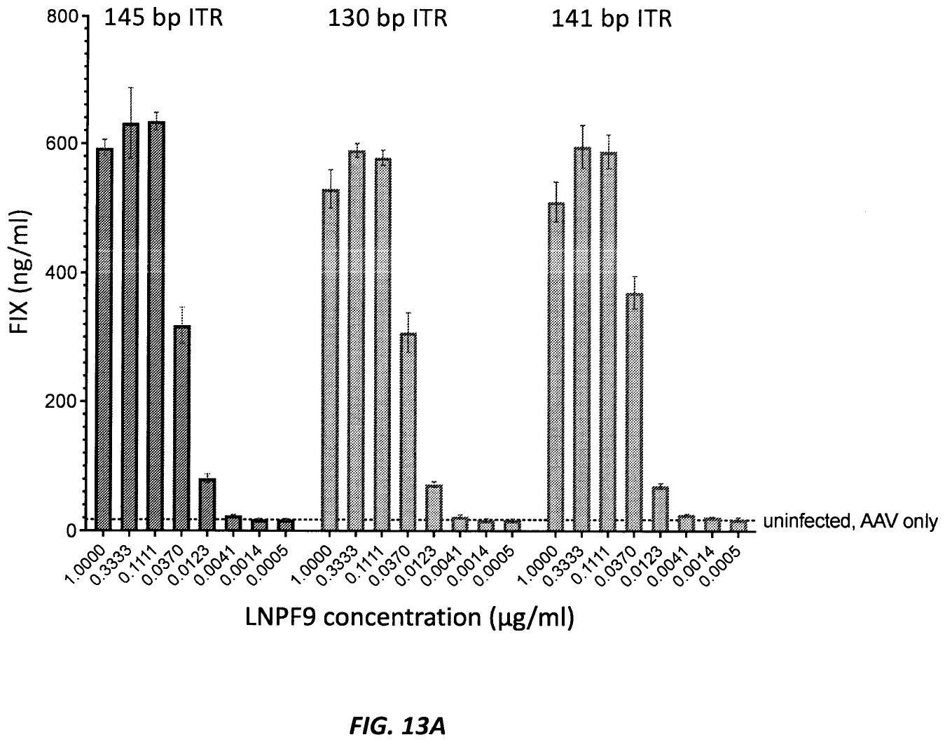

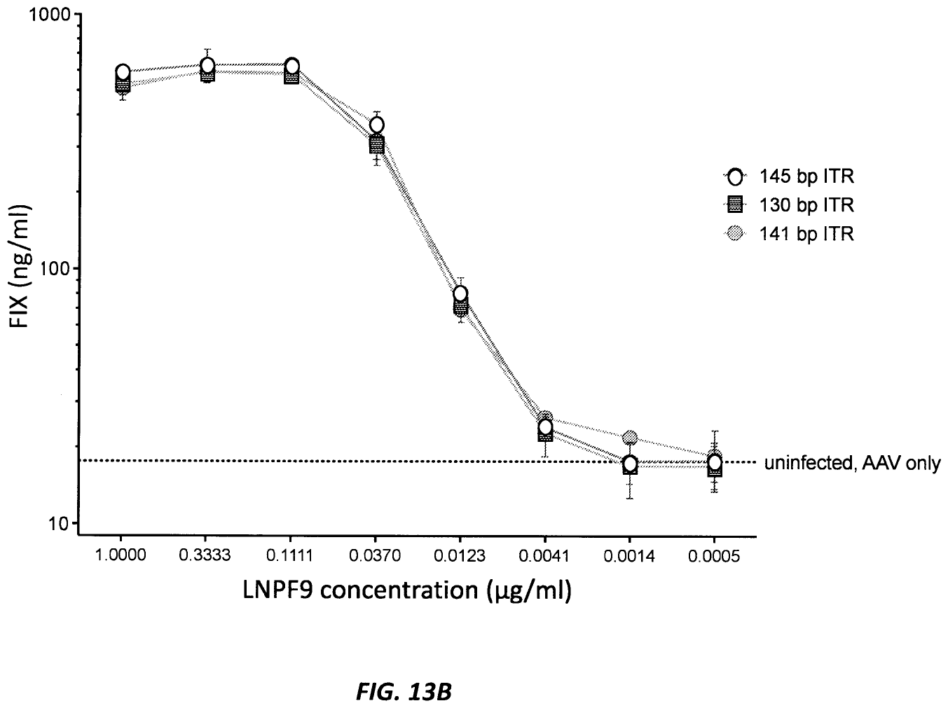

Targeted insertion of F9 DNA templates into the ALB locus of primary human hepatocytes was tested with recombinant AAV8 viruses that were produced using various length ITR length plasmids to determine the effects on FIX protein expression in vitro. Specifically, recombinant AAV8 viruses were produced using plasmids with 145 bp ITRs (SEQ ID NO: 196), 141 bp ITRs (SEQ ID NO: 197), or 130 bp ITRs (SEQ ID NO: 198). LNPF9 was administered at various concentrations to primary human hepatocytes together with rAAV8-packaged F9 DNA template Insert 30 with different ITR lengths (2.8E10 vg/μL rAAV8-F9-145 bp ITR, 3.24E10 vg/μL rAAV8-F9-130 bp ITR, and 1.33E10 vg/μL rAAV8-F9-141 bp ITR). FIX expression was then assessed at Day 7 by ELISA. As shown in FIGS. 13A-13B, rAAV8 viruses produced using various ITR length plasmids performed similarly in human hepatocytes, producing similar levels of FIX expression.

### Example 7. CRISPR/Cas9-Inserted Human F9 Gene Restores Hemostasis

The combination of LNPF9 (g9860) and REGV131 (Insert 30) was evaluated for expression and secretion of human FIX (hFIX) capable of restoring hemostasis (i.e., suppressing bleeding) following a tail bleed challenge in genetically manipulated mice harboring the humanized ALB locus and lacking FIX activity attributed to a F9 gene deficiency, similar to hemophilia B patients who have either a deficiency or a dysfunction of FIX.

The ALB^{hu/hu}/F9^{-/-} mice were dosed intravenously with LNPF9 (0.3 or 1.0 mg/kg) and/or REGV131 (1.5E13 or 1.5E12 vg/kg) as shown in Table 19.

TABLE 19
Treatment Groups.

| Group | Treatment | AAV (vg/kg) | LNP (mpk) | “n” size |
|---|---|---|---|---|
| A | Wild-type (ALB^{hu/hu}/F9^{+/+}) | — | — | 5 |
| B | No treatment (ALB^{hu/hu}/F9^{-/-}) | — | — | 5 |
| C | LNP1265 only | — | 1 | 5 |
| D | AAV only (REGV131) | 1.5e13 | — | 5 |
| E | LNP + AAV | 1.5e13 | 1 | 5 |
| F | LNP + AAV | 1.5e12 | 1 | 5 |
| G | LNP + AAV | 1.5e13 | 0.3 | 5 |
| H | LNP + AAV | 1.5e12 | 0.3 | 5 |

Plasma was isolated from mice at 1, 2, 4, and 6 weeks post-treatment, and mouse-generated hFIX was pulled-down using capture antibody specific for hFIX (AHIX-5041; Haematologic Technologies), and a chromogenic assay (BioPhen FIX, Hypen BioMed) was used to measure relative hFIX activity. Human FIX expression was assessed using MESO Scale Discovery (MSD). Biotinylated capture antibody was prepared by making 1 μg/mL of capture antibody in Assay Diluent (0.5% BSA-PBS) from the stock (Capture Mab HFIX R044 Biotin conjugated by MSD, 1000 μg/mL), and detection antibody was prepared by making 1 μg/mL of detection antibody in Assay Diluent (0.5% BSA) from the stock (Detection Mab AHIX-5041 Sulfo-tag conjugated by MSD, 1000 μg/mL). Standards were prepared, and samples were prepared at 1 to 500 and 1 to 1000 dilutions in Assay Diluent. Blocking was performed by adding 150 μL/well of blocking solution into the MSD GOLD plate, sealing and incubating the plate for 1 hour at room temperature with shaking (˜700 rpm). Coating was performed by tapping out the blocking solution by flicking the plate in the trash. Biotinylated capture antibody was added (25 μL/well of 1 μg/mL biotinylated capture antibody in Assay Diluent). The plate was sealed and incubated 4° C. overnight. The plate was washed in a plate washer (350 μL PBST×4). Calibration standards (50 μL) and diluted samples were added to the wells. The plate was sealed and incubated for 2 hours at room temperature with shaking (˜700 rpm) or at 4° C. overnight. For detection, the plate was washed in a plate washer (350 μL PBST×4), SULFO-TAG detection antibody was added (25 μL/well of 1 μg/mL SULFO-TAG detection antibody in Assay Diluent), and the plate was sealed and incubated for 1 hour at room temperature with shaking (˜700 rpm). The plate was washed in a plate washer, Read solution was added (150 μL/well of 2× MSD Read Solution), and the plate was immediately read on MESO SECTOR S 600MM (with Methodical Mind software).

Human FIX activity was assessed using the BIOPHEN Factor IX kit following immuno-precipitation of hFIX from mouse plasma. For preparation for immunoprecipitation of hFIX from plasma, an ELISA plate was coated with 100 ng Anti-Hu FIX antibody (monoclonal antibody from HTI-AHIX-5041) by diluting mAb in PBS at 1 μg/mL and then adding 100 μL diluted mAb onto a High-Bound assay plate at 100 ng/100 μL/well. The plate was incubated overnight at 4° C. The coating mAb solution was discarded. The coated plate was then blocked with 3% BSA/PBS by adding 150 μL/well on the plate to incubate for 2 hours at room temperature. The plate was then washed (4×400 μL PBS Wash Buffer). For preparation of study samples and standard/calibrator, the study plasma samples were diluted in R4 buffer from the kit. Calibrator plasma was generated from the kit using standard ranges of 100% to 0% according to the kit's protocol. (e.g., human normal pooled plasma purchased from George King Bio-Medical is 100× diluted with R4 buffer as 100% hFIX activity in assay). Standard calibrator (purified hFIX) and samples were diluted in R4 buffer (generated standard curve by using ERL purified plasma hFIX protein diluted in R4 buffer at 3× serial dilution from starting Std 1=100 ng/mL) and then added to the plate in 100 μL/well and incubated at room temperature for 2 hours (or overnight at 4° C.). The plate was then washed 3× with PBST. To trigger and quantify activity, all contents were discarded, and 50 μL of R4 was added to all of the test wells. The activity assay was then run as described in the kit protocol and as follows: added 50 μL R1 buffer (pre-warmed at 37° C.) and incubated for 4 minutes at 37° C.; added 50 μL R2 buffer (pre-warmed at 37° C.) and incubated for 8 minutes at 37° C.; added 50 μL R3 buffer (pre-warmed at 37° C.) and incubated for 5 minutes at 37° C.; and added 50 μL of 20% acetic acid (stop solution) and read absorbance at 405 nm in Molecular Device microplate reader by SoftMax 7.2.

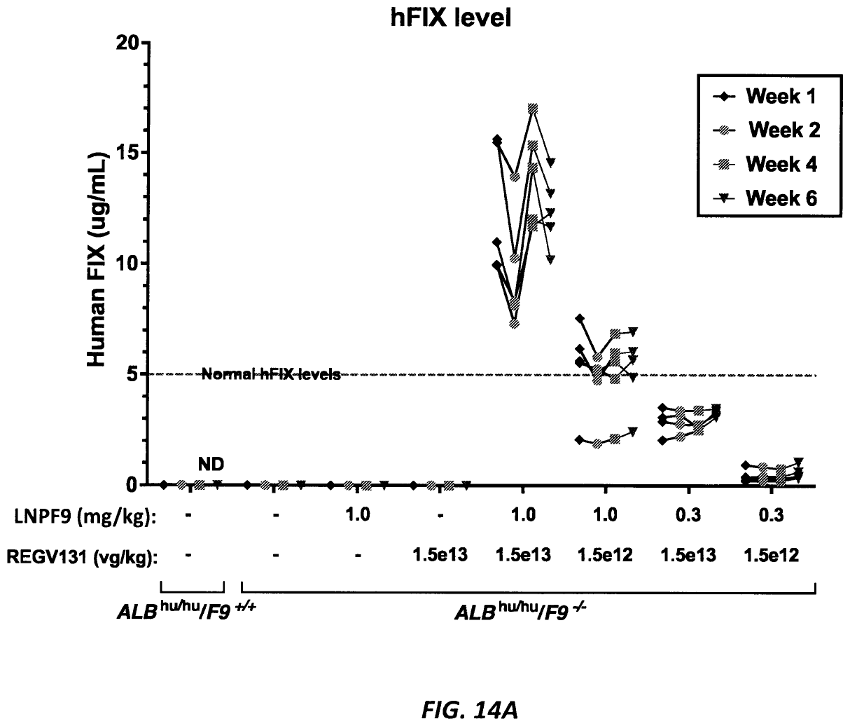

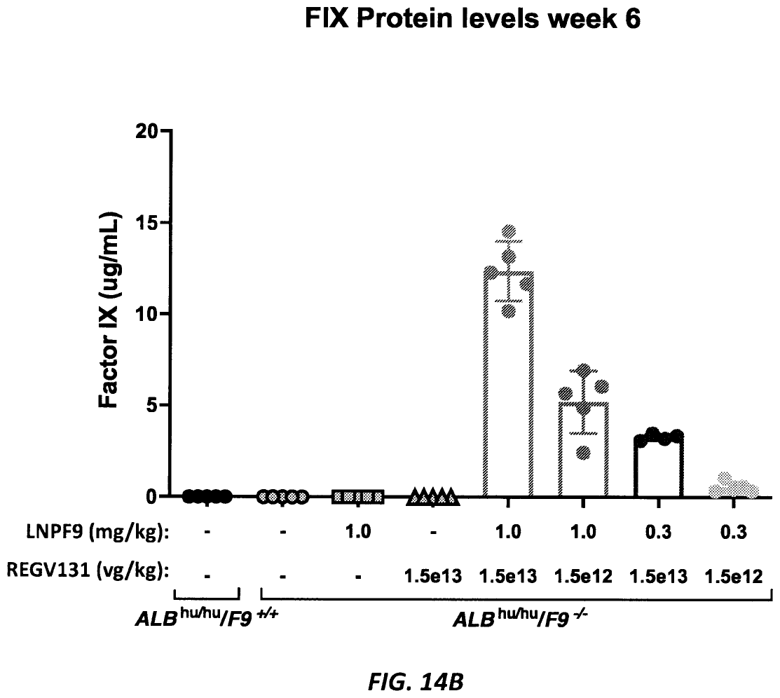

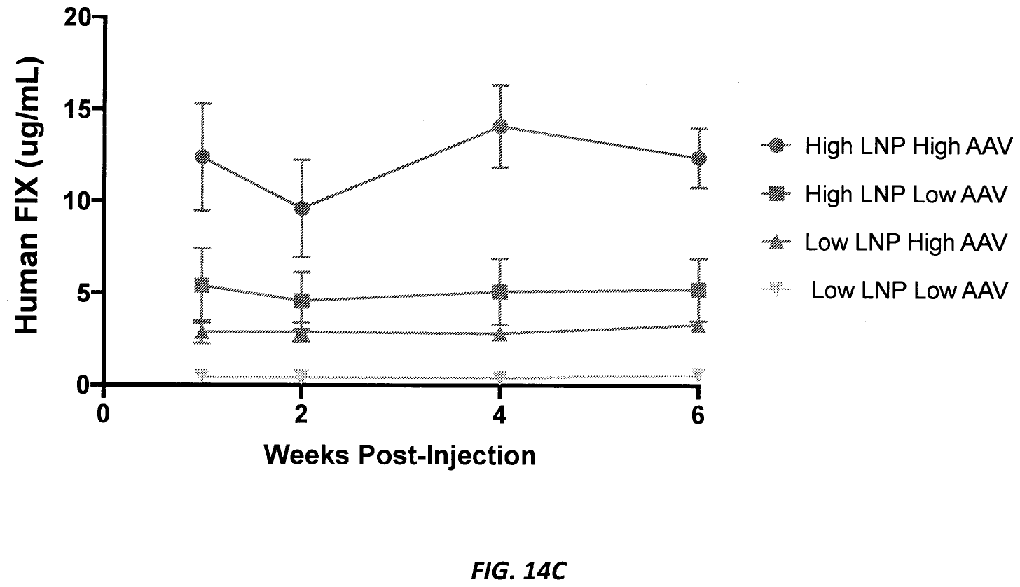

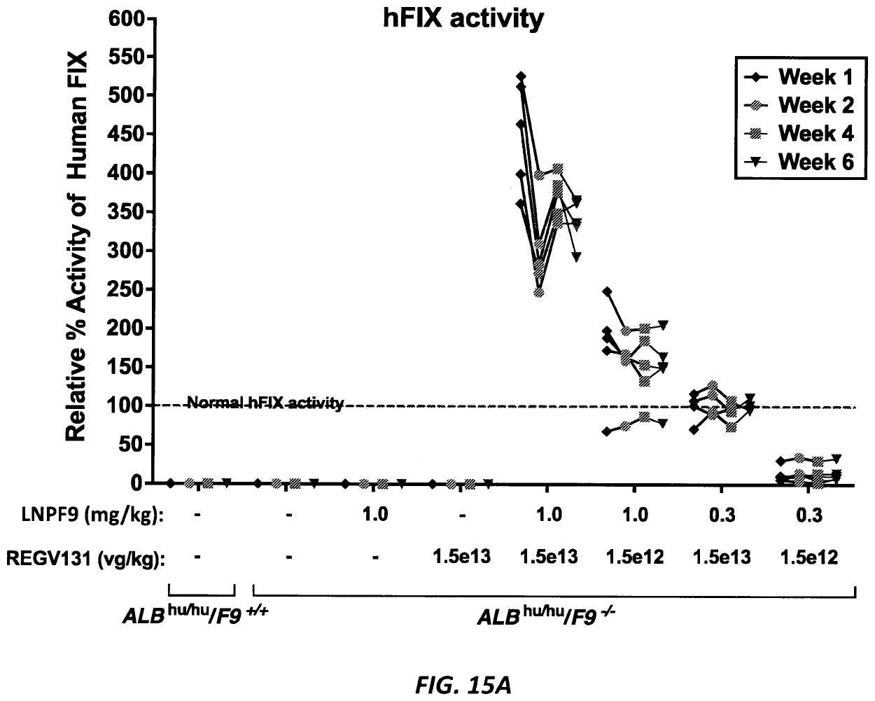

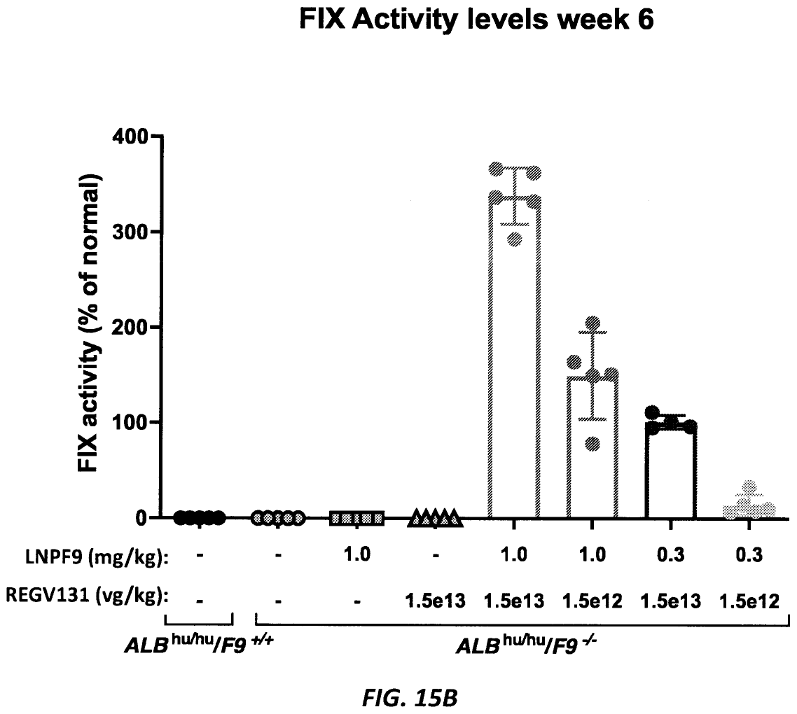

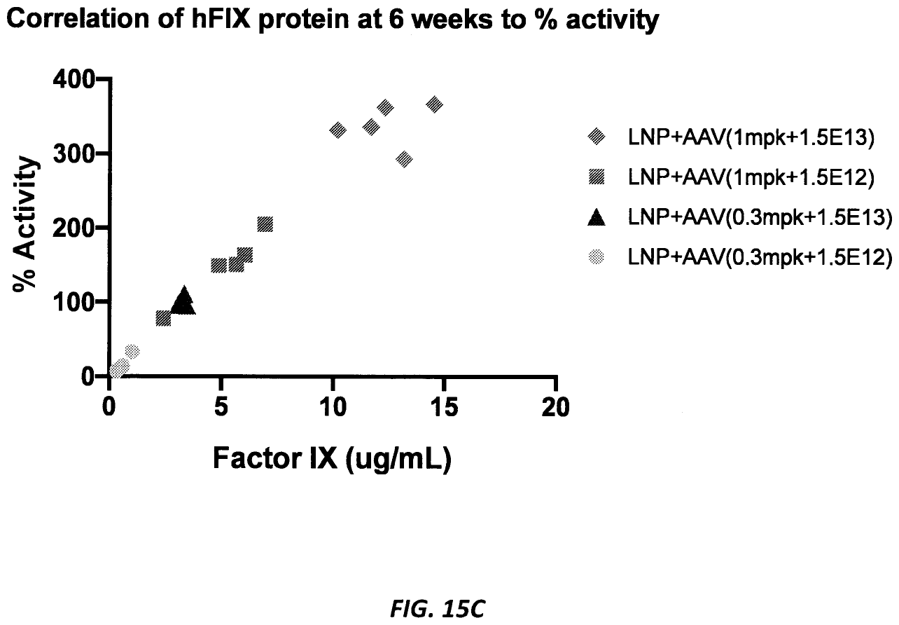

FIGS. 14A and 14C show hFIX expression levels in plasma at weeks 1, 2, 4, and 6, FIG. 14B shows hFIX expression levels in plasma at week 6, FIG. 15A shows hFIX activity in plasma at weeks 1, 2, 4, and 6, and FIG. 15B shows hFIX activity in plasma at week 6. FIG. 15C shows correlation of hFIX expression levels in plasma to hFIX activity. As shown in FIGS. 14A-15C, the REGV131-LNPF9 treatment resulted in dose-responsive hFIX expression and activity in ALB^{hu/hu}/F9^{-/-} mice through the six weeks tested. Levels of insertion of the insertion template (Insert 30) and mRNA levels of hFIX showed a similar dose response (data not shown).

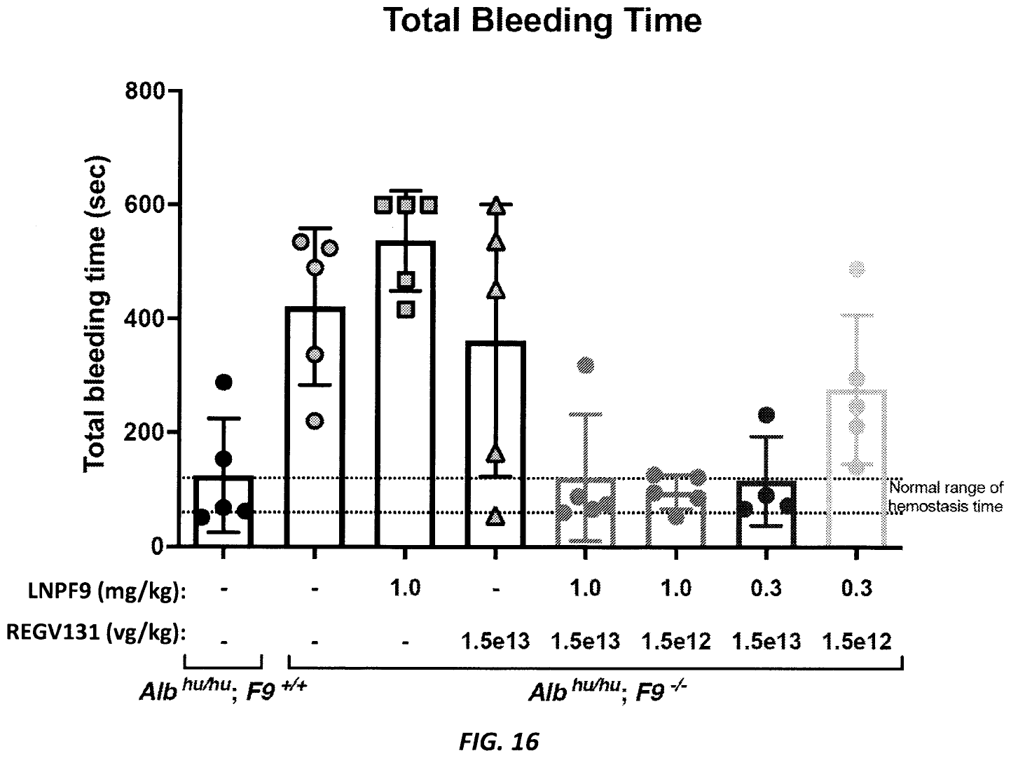

Bleeding time after tail cut was measured at 4 weeks to assess in vivo hemostasis. A temperature controlled 37° C. mini-incubator was prepared with a nose cone ready for isoflurane anesthesia. For each mouse, lidocaine hydrochloride (2%) was applied to the tip of tail for analgesia. The mouse was placed into the heated 37° C. isoflurane chamber for 5 minutes with 2% isoflurane and 1.5 L/min rate of 100% oxygen. Once the mouse became unconscious (˜5 min), the mouse was placed on top of a heated pad to maintain body temperature and maintain anesthesia via nosepiece. Buprenorphine-SR/ER (1 mg/mL) was administered at 1 mg/kg subcutaneously for analgesia. Povidone gauze was used to wipe the tail tip, followed by an alcohol pad; this was repeated two more times using new povidone and alcohol pads each time. A ruler or caliper was used to measure out 2 mm from the tip and/or 1 mm diameter, and the tail was cut using a sterile scalpel blade. The mouse was immediately transferred into the temperature controlled (37° C.) mini-incubator, the mouse's nose was put into the isoflurane nose cone, and the tail tip was placed into 5 mL saline (1 cm deep from tail tip). Blood flow was closely monitored for time to first hemostasis. Time to first hemostasis time refers to bleeding time without rebleeding for a minute after tail is cut. Total bleeding time refers to the sum of initial bleeding time plus any additional rebleeding times during 10 minutes after tail cut. Blood flow was continuously monitored for up to 10 minutes for rebleeding, and all rebleeding times were recorded. After 10 minutes, the mouse was removed from the mini-incubator, and the tail tip was pressed with a sterilized gauge pad to make sure bleeding stopped completely. Clotisol (blood clotting suspension) was applied to the tail cut site if necessary. Saline (0.5 mL of 0.9% saline) was injected subcutaneously, and the animal was placed back into its cage and monitored until it fully recovered from anesthesia. The total bleeding time for each group is shown in FIG. 16. As shown in FIG. 16, the REGV131-LNPF9 treatment resulted in rescue of hemostasis capacity at 4 weeks.

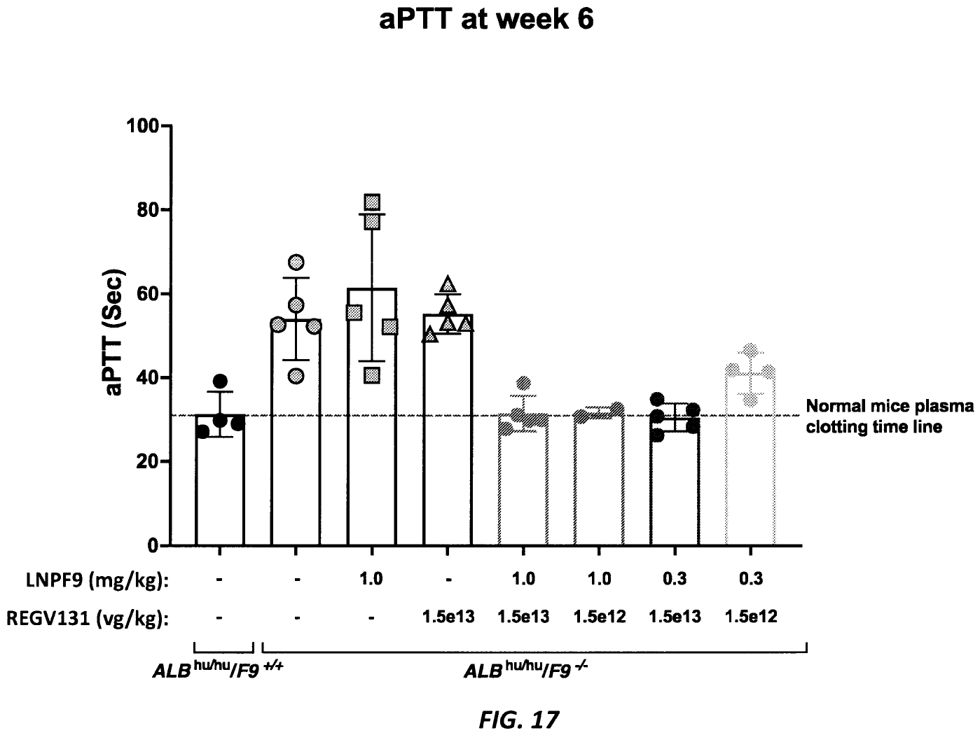

Activated partial thromboplastin time (aPTT) was assessed in undiluted plasma at week 6. As shown in FIG. 17, the REGV131-LNPF9 treatment in ALB^{hu/hu}/F9^{-/-} mice normalized aPTT to values observed in ALB^{hu/hu}/F9^{+/+} mice.

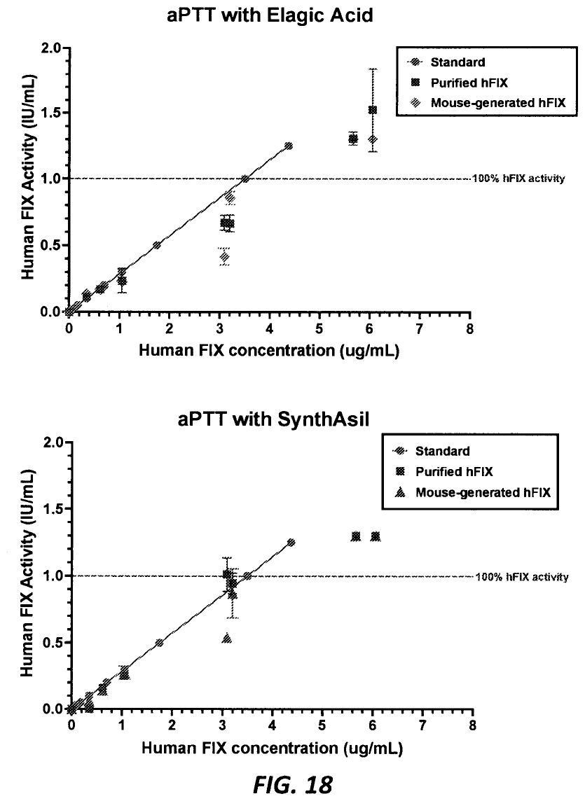

Human FIX activity was then assessed using a clinical one-stage clotting/aPTT assay with ellagic acid or SynthAsil. Standard/calibrator plasma from the same preparation of purified human FIX proteins (from ERL HFIX 1009, Lot4940) spiked in ALB^{hu/hu}/F9^{-/-} mice plasma was used for preparing the standard curves from 1.25 IU/mL to 0 IU/mL of human FIX in KO mice plasma. Control plasma samples were generated by matching up the same concentration of hFIX protein in F9^{-/-} mice plasma as the selected mice plasma samples from the insertion study. Following preparation of study standard/calibrator and control sample, all study plasma samples were 10× diluted with human FIX deficient plasma (purchased from PrecisionBioLogic, Cat #FDP09-15). A ball was then dispensed to each cuvette in the cuvette-strip. Cuvette-strips were placed in the incubation area on Start4 Hemostasis analyzer (Diagnostica Stago, Manual Ref 0931079A) for pre-warming machine at 37° C. Diluted plasma sample was added (50 μL/cuvette in duplicates) on the cuvettes of the Start4 Hemostasis analyzer, and test cuvettes were incubated at 37° C. for 3 minutes. Ellagic acid (APTT-XL Thermo Scientific, Cat #95059-804) or SynthAsil (HemosIL, APTT Lyophilized silica—0008468710) was added (50 μL/cuvette) and incubated at 37° C. for 5 minutes. On the Test-Column, pipette control key was activated (the ball starts moving back and forth in the cuvette), and the pre-warmed 50 μL of 0.025 M CaCl₂) was dispensed to each cuvette. When the clot formed, the ball stopped, and the clotting time was recorded. Data analysis was by GraphPad Prism 8 software, using nonlinear best curve fit to get interpolated data from the standard curve. As shown in FIG. 18, mouse-generated human FIX demonstrated specific activity similar to purified human FIX in the one-stage clotting/aPTT assays.

Human FIX activity was then assessed using a clinical two-stage chromogenic substrate assay kit by Aniara Diagnostica. Standard/calibrator plasma from the same preparation of purified human FIX proteins (from ERL HFIX 1009, Lot4940) spiked in ALB^{hu/hu}/F9^{-/-} mice plasma was used for preparing the standard curves from 1.25 IU/mL to 0 IU/mL of human FIX in KO mice plasma. Control plasma samples were generated by matching up the same concentration of hFIX protein in F9^{-/-} mice plasma as the selected mice plasma samples from the insertion study. Following preparation of study standard/calibrator and control sample, all study plasma samples were 100× diluted with diluted with R4 buffer, and 25 μL/well was added to the 96 well assay plates in duplicates. Next, 25 μL/well R1 Reagent was added to above assay plates and incubated at 37° C. for 5 minutes, 25 μL/well R2 reagent (pre-warmed at 37° C.) was added and plates were incubated at 37° C. for 8 minutes, 25 μL/well R3 reagent was added and assay plates were incubated for 3-5 minutes at 37° C., and 25 μL/well of 20% acetic acid (stop solution) was added and absorbance at 405 nm was read in a Molecular Device microplate reader by SoftMax 7.2. Data analysis was by GraphPad Prism 8 software, using nonlinear best curve fit to get interpolated data from the standard curve.

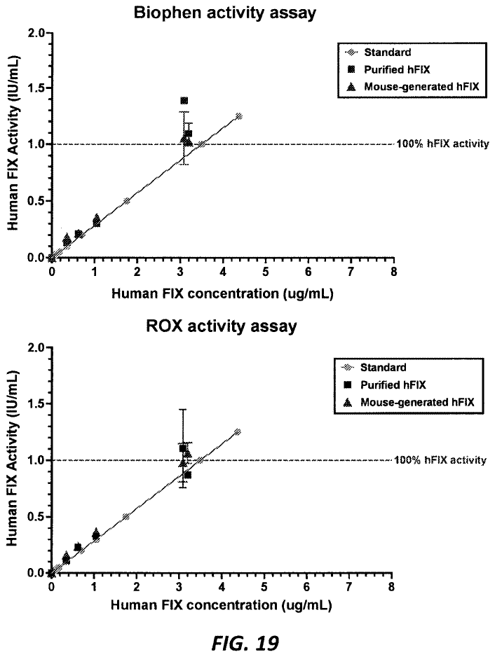

In a separate experiment, human FIX activity was then assessed using a clinical two-stage chromogenic substrate assay kit by Rossix. Standard/calibrator plasma from the same preparation of purified human FIX proteins (from ERL HFIX 1009, Lot4940) spiked in ALB^{hu/hu}/F9^{-/-} mice plasma was used for preparing the standard curves from 1.25 IU/mL to 0 IU/mL of human FIX in KO mice plasma. Control plasma samples were generated by matching up the same concentration of hFIX protein in F9^{-/-} mice plasma as the selected mice plasma samples from the insertion study. Following preparation of study standard/calibrator and control sample, all study plasma samples were 80× diluted with Diluent buffer, and 13 μL/well was added to the 96 well assay plates in duplicates. Next, 13 μL/well Reagent A was added to above assay plates and incubated at 37° C. for 5 minutes, 75 μL/well Reagent B (pre-warmed reagent at 37° C.) was added and plates were incubated at 37° C. for 8 minutes (for activation), 25 μL/well FXa substrate was added and assay plates were incubated for 3 minutes at 37° C., and 25 μL/well of 20% acetic acid (stop solution) was added, and absorbance at 405 nm was read in a Molecular Device microplate reader by SoftMax 7.2. Data analysis was by GraphPad Prism 8 software, using nonlinear best curve fit to get interpolated data from the standard curve. As shown in FIG. 19, mouse-generated human FIX demonstrated specific activity similar to purified human FIX in the two-stage chromogenic substrate assays, using both the clinical two-stage chromogenic substrate assay kit by Aniara Diagnostica and the clinical two-stage chromogenic substrate assay kit by Rossix.

In summary, dose-dependent hFIX expression was observed with increasing concentrations of LNP and AAV, and expression was sustained over the six weeks of observation. In addition, the mouse-generated hFIX was functionally active, as verified using multiple functional assays: it restored aPTT to normal, it had a similar specific activity as purified hFIX (confirmed using OS and CS clinical assays), and it restored hemostasis capacity following tail bleed challenge.

1. A composition comprising a nucleic acid construct comprising a first factor IX protein coding sequence or a reverse complement of the first factor IX protein coding sequence.

2.-64. (canceled)

65. The composition of claim 1, further comprising a nuclease agent that targets a nuclease target site in a target gene.

66.-146. (canceled)

147. A cell comprising the composition of claim 1.

148.-159. (canceled)

160. A method of introducing a factor 9 nucleic acid into a cell, comprising administering the composition of claim 1 to the cell.

161. A method of integrating a factor 9 nucleic acid construct into a target gene in a cell, comprising administering the composition of claim 65 to the cell,
wherein the nuclease agent cleaves the nuclease target site in the target gene to create a cleavage site, the nucleic acid construct is inserted into the cleavage site to create a modified target gene, and factor IX protein is expressed from the modified target gene.

162. A method of expressing factor IX in a cell, comprising administering the composition of claim 65 to the cell,
wherein the nuclease agent cleaves the nuclease target site in the target gene to create a cleavage site, the nucleic acid construct is inserted into the cleavage site to create a modified target gene, and factor IX protein is expressed from the modified target gene.

163.-177. (canceled)

178. A method of treating a factor IX deficiency in a subject, comprising administering the composition of claim 65 to the subject.

179. A method of treating hemophilia B in a subject, comprising administering the composition of claim 65 to the subject.

180. A method of preventing or inhibiting spontaneous bleeding in a subject having hemophilia B, comprising administering the composition of claim 65 to the subject.

181.-216. (canceled)

217. A method of inserting a nucleic acid encoding factor IX protein into a target genomic locus in a neonatal cell or a population of neonatal cells, comprising administering to the neonatal cell or the population of neonatal cells:
(a) a nucleic acid construct comprising a factor IX protein coding sequence; and
(b) a nuclease agent or one or more nucleic acids encoding the nuclease agent, wherein the nuclease agent targets a nuclease target site in the target genomic locus,
wherein the nuclease agent cleaves the nuclease target site, and the nucleic acid construct is inserted into the target genomic locus.

218. A method of expressing a factor IX protein from a target genomic locus in a neonatal cell or a population of neonatal cells, comprising administering to the neonatal cell or the population of neonatal cells:
(a) a nucleic acid construct comprising a factor IX protein coding sequence; and
(b) a nuclease agent or one or more nucleic acids encoding the nuclease agent, wherein the nuclease agent targets a nuclease target site in the target genomic locus,
wherein the nuclease agent cleaves the nuclease target site, the nucleic acid construct is inserted into the target genomic locus to create a modified target genomic locus, and the factor IX protein is expressed from the modified target genomic locus.

219.-227. (canceled)

228. A method of inserting a nucleic acid encoding a factor IX protein into a target genomic locus in a neonatal cell in a neonatal subject, comprising administering to the neonatal subject:
(a) a nucleic acid construct comprising a factor IX protein coding sequence; and
(b) a nuclease agent or one or more nucleic acids encoding the nuclease agent, wherein the nuclease agent targets a nuclease target site in the target genomic locus,
wherein the nuclease agent cleaves the nuclease target site, and the nucleic acid construct is inserted into the target genomic locus.

229. A method of expressing a factor IX protein from a target genomic locus in a neonatal cell in a neonatal subject, comprising administering to the neonatal subject:
(a) a nucleic acid construct comprising a factor IX protein coding sequence; and
(b) a nuclease agent or one or more nucleic acids encoding the nuclease agent, wherein the nuclease agent targets a nuclease target site in the target genomic locus,
wherein the nuclease agent cleaves the nuclease target site, the nucleic acid construct is inserted into the target genomic locus to create a modified target genomic locus, and the factor IX protein is expressed from the modified target genomic locus.

230.-232. (canceled)

233. A method of treating a factor IX deficiency in a neonatal subject in need thereof, comprising administering to the neonatal subject:
(a) a nucleic acid construct comprising a factor IX protein coding sequence; and
(b) a nuclease agent or one or more nucleic acids encoding the nuclease agent, wherein the nuclease agent targets a nuclease target site in a target genomic locus,
wherein the nuclease agent cleaves the nuclease target site, the nucleic acid construct is inserted into the target genomic locus to create a modified target genomic locus, and the factor IX protein is expressed from the modified target genomic locus.

234. A method of preventing or inhibiting spontaneous bleeding in a neonatal subject having hemophilia B, comprising administering to the neonatal subject:
(a) a nucleic acid construct comprising a factor IX protein coding sequence; and
(b) a nuclease agent or one or more nucleic acids encoding the nuclease agent, wherein the nuclease agent targets a nuclease target site in a target genomic locus,
wherein the nuclease agent cleaves the nuclease target site, the nucleic acid construct is inserted into the target genomic locus to create a modified target genomic locus, and the factor IX protein is expressed from the modified target genomic locus and prevents or inhibits spontaneous bleeding in the neonatal subject.

235. (canceled)

236. A method of treating hemophilia B in a neonatal subject in need thereof, comprising administering to the neonatal subject:
(a) a nucleic acid construct comprising factor IX protein coding sequence; and
(b) a nuclease agent or one or more nucleic acids encoding the nuclease agent, wherein the nuclease agent targets a nuclease target site in a target genomic locus,
wherein the nuclease agent cleaves the nuclease target site, the nucleic acid construct is inserted into the target genomic locus to create a modified target genomic locus, and the factor IX protein is expressed from the modified target genomic locus, thereby treating the hemophilia B.

237.-406. (canceled)

407. A neonatal cell or a population of neonatal cells made by the method of claim 217.

408. A neonatal cell or a population of neonatal cells comprising a nucleic acid construct comprising a factor IX protein coding sequence inserted into a target genomic locus.

409.-454. (canceled)
